(19) United States
(12) Patent Application Publication
(10) Pub. No.: US 2024/0174662 A1
(43) Pub. Date: May 30, 2024

(54) SMALL MOLECULE INHIBITORS OF SALT INDUCIBLE KINASES

(71) Applicants: Janssen Biotech, Inc., Horsham, PA (US); The Broad Institute, Inc., Cambridge, MA (US); The General Hospital Corporation, Boston, MA (US)

(72) Inventors: Genesis M. BACANI, Horsham, PA (US); Wenying CHAI, Horsham, PA (US); De Michael CHUNG, Horsham, PA (US); Steven D. GOLDBERG, Horsham, PA (US); Gavin HIRST, Horsham, PA (US); Virendar KAUSHIK, Cambridge, MA (US); Eduardo V. MERCADO-MARIN, Horsham, PA (US); Donald RAYMOND, Cambridge, MA (US); Mark SEIERSTAD, Horsham, PA (US); Russel C. SMITH, Horsham, PA (US); Thomas SUNDBERG, Cambridge, MA (US); Mark S. TICHENOR, Horsham, PA (US); Jennifer D. VENABLE, Horsham, PA (US); Jianmei WEI, Horsham, PA (US); Ramnik XAVIER, Cambridge, MA (US)

(21) Appl. No.: 18/263,432

(22) PCT Filed: Jan. 31, 2022
(86) PCT No.: PCT/US2022/070432

§ 371 Date: Jul. 28, 2023

Related U.S. Application Data
(60) Provisional application No. 63/144,079, filed on Feb. 1, 2021.

(51) Int. Cl. C07D 471/04; C07D 519/00

(52) U.S. Cl. CPC C07D 471/04; C07D 519/00

(57) ABSTRACT

Small molecule inhibitors of salt inducible kinases (SIKs) are provided. In particular, compounds of formula (II) and compounds of formula (III), and tautomers, stereoisomers, pharmaceutically acceptable salts and solvates thereof are provided. Also provided are pharmaceutical compositions containing the compounds, methods of preparing the compounds, and methods of using the compounds for inhibiting SIKs, such as SIK1 and SIK2, and methods of treating diseases mediated by SIKs.

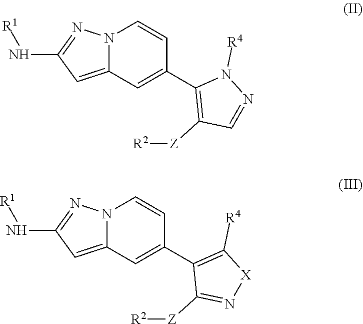

### CROSS REFERENCE TO RELATED APPLICATION

This application claims priority to U.S. Provisional Application No. 63/144,079 filed on Feb. 1, 2021, the disclosure of which is incorporated herein by reference in its entirety.

### FIELD

The present invention relates to the field of chemistry and medicine. More particularly, the present invention relates to salt-inducible kinases inhibitors and their use in medical treatment.

### BACKGROUND

A protein kinase is an enzyme that catalyzes the transfer of phosphate groups to proteins or other organic molecules. The salt-inducible kinases (SIKs) are Ser/Thr kinases members of the Adenosine Monophosphate-Activated Kinase (AMPK) subfamily of kinases and three isoforms have been described (i.e., SIK1, SIK2 (QIK), and SIK3 (QSK) (1). Small molecule protein kinase inhibitors are useful for treating disease including, but not limited to, proliferative diseases (cancer, benign neoplasms, pathological angiogenesis), immune disorders (auto-inflammatory disease, autoimmune disease), and disorders of the musculoskeletal system. For example, the pan-Janus kinase inhibitor tofacitinib is approved to treat rheumatoid arthritis and ulcerative colitis. There is a need for small molecule kinase inhibitors that selectively inhibit SIK1, SIK2 and/or SIK3.

Crohn's disease and ulcerative colitis are the two major forms of inflammatory bowel disease (IBD), a disorder characterized by chronic inflammation of the gastrointestinal (GI) tract. There is an urgent need to discover new therapies for these disorders as broadly immunosuppressive therapies used to treat IBD are not always effective or durable, and can cause severe side effects (2). Aberrant host responses to the intestinal microbiota in genetically susceptible individuals disrupts immune homeostasis, leading to elevated levels of proinflammatory cytokines such as tumor necrosis factor (TNF) and interleukin (IL-12) and the development of IBD. In the gastrointestinal tract, homeostasis is an active process that requires a careful balance of host responses to the enteric luminal contents (3), a process for which the immunoregulatory cytokine IL-10 is vital (4). Single Nucleotide Polymorphisms (SNPs) in the genetic loci containing the anti-inflammatory cytokine IL10 or its receptor (IL10RA) are associated with increased risk of Crohn's disease and ulcerative colitis (5), and loss-of-function mutations in the coding regions of these genes lead to a rare but severe, pediatric-onset enterocolitis in affected individuals. This phenotype is recapitulated in mice deficient in either Il10^{-/-} or Il10ra^{-/-}, which develop spontaneous colitis (4, 6). Together, these genetic data suggest that IBD patients may benefit from therapies that augment IL-10 function specifically in the intestinal mucosa.

Approaches to target IL-10 to inflamed tissues hold therapeutic promise (7). For example, oral administration of bacteria (Lactococcus lactis) engineered to express IL-10 resulted in beneficial, though not persistent, effects in Crohn's disease patients (8). In addition, a fusion protein linking recombinant IL-10 to an antibody targeting an inflammation associated integrin (dekavil) is well tolerated in initial clinical safety trials (9), (10). Similarly, small molecules that promote IL-10 production by aberrantly activated intestinal immune cells may represent a viable approach to dampen intestinal inflammation in IBD patients while avoiding the limitations of systemically administering recombinant IL-10.

Several mechanistic classes of small molecules have been shown to enhance IL-10 production in activated myeloid cells, such as stimulating cAMP Response Element-Binding Protein (CREB) signaling with prostanoid receptor agonists (11), phosphodiesterase (PDE) inhibitors (12-14); disrupting signaling to NF-κB activation with inhibitors of protein kinase C (PKC) or glycogen synthase kinase-30 (GSK-30) (15, 16); and increasing acetylated α-tubulin levels with paclitaxel or the HDAC6 inhibitor tubastatin A, reported to promote IL-10 production via p38 MAP kinase (17). However, these approaches either do not produce the desired outcome or are associated with potential liabilities (e.g., prostanoid receptor agonist like PGE2 do not show selectivity for IL-10 induction (13), and potent emetic effects have hampered development of PDE inhibitors to treat IBD (18).

Small molecule SIK inhibitors may hold particular therapeutic potential for treatment of IBD because SIK inhibitors both enhance IL-10 production and reduce production of inflammatory cytokines, including TNF and IL-12, in both activated murine dendritic cells and monocyte-derived human macrophages and dendritic cells from healthy volunteers (13), (19). In addition, direct injection of a small molecule SIK inhibitor enhances levels of IL-10 and reduces levels of TNF in the serum and colon of mice during the acute inflammatory response induced by stimulation with lipopolysaccharide (20). The ability of SIK inhibitors to coordinately up-regulate IL-10 and suppress inflammatory cytokines is supported by studies in SIK1 and SIK2 kinase dead knock-in mice; Dendritic cells derived from Sik1KI, Sik2KI double mutant mice secreted elevated levels of IL-10 and diminished levels of TNF and IL-12/23p40 in response to LPS (21). Therefore, small molecule SIK inhibitors represent a potential therapeutic strategy to enhance IL-10 production and concomitantly suppress production of pro-inflammatory cytokines by immune cells in the inflamed intestinal mucosa of IBD patients.

In quiescent myeloid cells, the SIKs phosphorylate the CREB Regulated Transcription Coactivator (CRTC3)-3 as well as Histone Deacetylases (HDAC)-4 and HDAC5, which results in their cytosolic sequestration by binding to 14-3-3 proteins (12, 22, 23). Activation of Protein Kinase A (PKA) following prostanoid receptor stimulation inhibits SIK activity, enabling CRTC3 to enter the nucleus and stimulate transcription of CREB targets like IL-10 (12, 22). In addition, translocation of HDAC4 and HDAC5 into the nucleus in response to small molecule SIK inhibitors has been shown to suppress inflammatory cytokine production by deacetylation of NF-κB subunits and histone lysines near promoters of several pro-inflammatory cytokines (24).

SIK2 is a centrosome kinase and has a role in bipolar mitotic spindle formation in some cancers (25). Elevated levels of SIK2 protein is present in approximately 30% of serous ovarian cancer relative to normal ovarian epithelium tissue (26). Suppressing SIK2 function by depleting SIK2 transcript with targeting siRNAs or inhibiting SIK2 function with a small molecule pan-SIK inhibitor disrupts mitosis of ovarian cancer cell lines and increases sensitivity to paclitaxel in cell culture and xenograft studies (25, 26). Additionally, SIK3 has been identified as a dependency for a subset of acute myeloid leukemias (AMLs) (27). AML lines that are susceptible to SIK3 knockout are driven by mixed lineage leukemia (MLL) fusion oncoproteins, which creates a dependency on the MEF2C transcription factor. SIK3 activity is required to maintain inhibitory phosphorylation on HDAC4 and HDAC5. When SIK3 activity is reduced, HDAC4 and HDAC5 are relieve of inhibitory phosphorylation resulting in suppression of MEF2C function. CRISPR/Cas9-based knockout of SIK3 or inhibiting SIK3 function with a small molecule pan-SIK inhibitor suppresses proliferation of MEF2C-dependent AMLs (27).

### BRIEF SUMMARY

Accordingly, there is a need in the art for novel inhibitors of salt inducible kinases (SIKs). Small molecule inhibitors of SIKs, such as SIK1 and SIK2, are provided.

In a general aspect, provided are small molecule inhibitors of salt inducible kinases (SIKs)

In an embodiment, provided is a compound of formula (II):

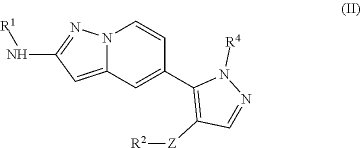

or a stereoisomer, tautomer, pharmaceutically acceptable salt or solvate thereof,
wherein:
   Z is —O—(CH₂)ₙ—, provided that O is directly bonded to the pyrazolyl;
   n is 0 or 1;
   R⁴ is —CH₃, —CH₂CH₃, —CHF₂, —CD₃, —CH₂-cyclopropyl or benzyl;
R¹ is —C(O)R⁵, phenyl, or a 5- to 6-membered heteroaryl having 1-2 nitrogen heteroatoms, or

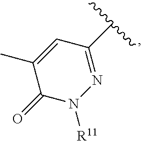

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl, wherein the 5- to 6-membered heteroaryl is optionally fused to a ring selected from the group consisting of benzene, cyclopentane, tetrahydropyran, and a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, —CN, and phenyl;
R² is:
   a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH₃, —OCF₃, —CN, cyclopropyl, oxetanyl, tetrahydrofuranyl, pyrazolyl, and phenyl,
   a C₁₋₅ alkyl, wherein the C₁₋₅ alkyl is optionally substituted with one or more substituents independently selected from the group consisting of —OH, —N(R¹⁰)₂, C₁ fluoroalkyl, —COOR⁸, —CH₃, C₃-C₆ cycloalkyl, phenyl, pyridyl, 3-bicyclo[1.1.1]pentanyl, and —CN, wherein the C₃-C₆ cycloalkyl is optionally substituted with one or more —CH₃, or —NHCH₃, or a C₃-C₆ cycloalkyl, wherein the C₃-C₆ cycloalkyl is optionally substituted with one or more —OH;
each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F;
each R⁹ is independently C₁ fluoroalkyl or —CH₃;
each R¹⁰ is independently hydrogen, cyclopropyl, cyclopentyl, or —CH₃;
each R¹¹ is independently hydrogen, C₁₋₅ alkyl, or —CH₂-cyclopropyl; and
m is 1 or 2.

In some embodiments, provided is a compound of formula (II), or a stereoisomer, tautomer, pharmaceutically acceptable salt or solvate thereof, wherein R⁴ is —CH₃.

In some embodiments, provided is a compound of formula (II), or a stereoisomer, tautomer, pharmaceutically acceptable salt or solvate thereof, wherein Z is —O—.

In some embodiments, provided is a compound of formula (II), or a stereoisomer, tautomer, pharmaceutically acceptable salt or solvate thereof, wherein R² is azetidinyl optionally substituted with one or more substituents independently selected from the group consisting of —CH₃, —CH₂CH₃, —CH(CH₃)₂, —CH₂CH(CH₃)₂, —CD₃, —CD₂CD₃, —F, —CF₃, —CH₂CH₂F, —CH₂CH₂OF₃, —CH₂CH(OH)CH₃, —CH₂C(OH)(CH₃)₂, —CH₂C(OCH₃)(CH₃)₂, —C(O)CH₃, —CH₂C(O)CH₃, —CH₂CH₂—CN, —CH₂C(CN)(CH₃)₂, pyridinyl, phenyl, —CH₂-cyclopropyl, —CH₂-phenyl,

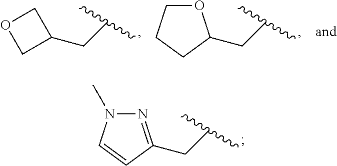

pyrrolidinyl optionally substituted with one or more substituents independently selected from the group consisting of —OCH₃, —CH₂CH₂—OCH₃, and —CH₃; morpholinyl; tetrahydrofuranyl optionally substituted with one or more —CH₃; a 6- to 9-membered bicyclic heterocyclyl selected from the group consisting of 3-oxa-9-azabicyclo[3.3.1]nonanyl, 7-oxa-2-azaspiro[3.5]nonanyl, 5-azaspiro[2.3]hexanyl, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein the 6- to 9-membered bicyclic heterocyclyl is optionally substituted with one or more substituents independently selected from the group consisting of —CH₃ and —CH₂CH₃; a C₁₋₅ alkyl optionally substituted with one or more substituents independently selected from the group consisting of —OH, —CH₃, —CF₃, —C₆H₅,

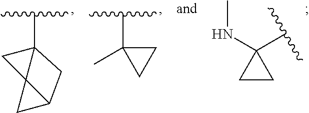

or a C₃-C₆ cycloalkyl, wherein the C₃-C₆ cycloalkyl is optionally substituted with one or more —OH.

In some embodiments, provided is a compound of formula (II), or a stereoisomer, tautomer, pharmaceutically acceptable salt or solvate thereof, wherein R¹ is —C(O)R⁵ and R⁵ is cyclopropyl optionally substituted with one or more substituents independently selected from the group consisting of —CH₃, —F, —CF₃, —CN, and phenyl.

In some embodiments, provided is a compound of formula (II), or a stereoisomer, tautomer, pharmaceutically acceptable salt or solvate thereof, wherein R¹ is pyrazinyl, pyridinyl, pyridazinyl, pyrimidinyl, or pyrazolyl, wherein each of the pyrazinyl, pyridinyl, pyridazinyl, pyrimidinyl, or pyrazolyl is optionally substituted with one or more substituents independently selected from the group consisting of —CH₃, cyclopropyl, CF₃, —CH₂-cyclopropyl, —OCHF₂, —COOCH₃, pyridinyl, and.

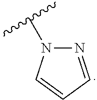 [SMILES: *n1cccn1]

In another embodiment, provided is a compound of formula (III):

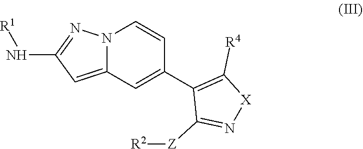

or a stereoisomer, tautomer, pharmaceutically acceptable salt or solvate thereof, wherein:
X is O or S;
Z is —O—(CH₂)ₙ—, provided that O is directly bonded to the isoxazolyl or isothiazolyl;
n is 0 or 1;
R⁴ is —CH₃ or —CH₂CH₃;
R¹ is —C(O)R⁵, phenyl, a 5- to 6-membered heteroaryl having 1-2 nitrogen heteroatoms, or

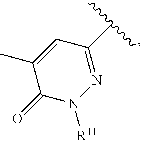

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, and —OCH₃, wherein the 5- to 6-membered heteroaryl is optionally fused to a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R² is:
   a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl, or
   a C₁₋₅ alkyl, wherein the C₁₋₅ alkyl is optionally substituted with one or more substituents independently selected from the group consisting of —OH, —N(R¹⁰)₂, C₁ fluoroalkyl, —COOR₈, —CH₃, C₃-C₆ cycloalkyl, phenyl, pyridyl, and —CN, wherein the phenyl is optionally substituted with one or more —F, or
   a C₃-C₆ cycloalkyl, wherein the C₃-C₆ cycloalkyl is optionally substituted with one or more —NH₂;
each R⁸ is independently hydrogen, cyclopropyl, or C₁₋₄ alkyl, wherein the C₁₋₄ alkyl is optionally substituted with one or more —F;
each R⁹ is independently C₁ fluoroalkyl or —CH₃;
each R¹⁰ is independently hydrogen or —CH₃; and
each R¹¹ is independently hydrogen, C₁₋₅ alkyl, or —CH₂-cyclopropyl; and
m is 1 or 2.

In some embodiments, provided is a compound of formula (III), or a stereoisomer, tautomer, pharmaceutically acceptable salt or solvate thereof, wherein X is O.

In some embodiments, provided is a compound of formula (III), or a stereoisomer, tautomer, pharmaceutically acceptable salt or solvate thereof, wherein R¹ is —C(O)R⁵ and R⁵ is cyclopropyl.

In some embodiments, provided is a compound of formula (III), or a stereoisomer, tautomer, pharmaceutically acceptable salt or solvate thereof, wherein R¹ is pyridazinyl or pyrimidinyl, wherein each of the pyridazinyl and pyrimidinyl is optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, and —CH₂OCH₃.

In some embodiments, provided is a compound of formula (III), or a stereoisomer, tautomer, pharmaceutically acceptable salt or solvate thereof, wherein R² is a C₁₋₅ alkyl optionally substituted with one or more substituents independently selected from the group consisting of —NH₂ and phenyl; or a 4- to 7-membered monocyclic heterocyclyl selected from the group consisting of pyrrolidinyl, morpholinyl, and azetidinyl, wherein the 4- to 7-membered monocyclic heterocyclyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), and phenyl; a 6- to 9-membered bicyclic heterocyclyl selected from the group consisting of 2-oxa-5-azabicyclo[2.2.1]heptanyl, 5-oxa-2-azabicyclo[2.2.1]heptanyl, 2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 2-oxa-5-azabicyclo[2.2.1]heptanyl, 5-oxa-2-azabicyclo[2.2.1]heptanyl, 2-azabicyclo[2.2.1]heptanyl, 9-azaspiro[3.5]nonanyl, and 3-(1,2,3,3a,5,6,7,7a-octahydropyrano[3,2-b]pyrrolyl is optionally substituted with one or more substituents independently selected from the group consisting of —CH₃ and —CH₂CH₃; or a C₃-C₆ cycloalkyl, wherein the C₃-C₆ cycloalkyl is optionally substituted with one or more —NH₂.

In particular embodiments, provided is a compound selected from the compounds of Examples 1-485, or a stereoisomer, tautomer, pharmaceutically acceptable salt or solvate thereof.

In another aspect, provided is a pharmaceutical composition comprising a compound as describe herein or a stereoisomer, tautomer, pharmaceutically acceptable salt or solvate thereof, and a pharmaceutically acceptable carrier.

In another aspect, provided is a method of inhibiting a salt inducible kinase (SIK) in a subject in need thereof, the method comprising administering to the subject a pharmaceutical composition as described herein. In certain embodiment, the subject is in need of a treatment of a disease, disorder, or condition mediated by a SIK, such as an autoimmune disorder or a proliferative disorder.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure.

Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification.

All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible subranges, all individual numerical values within that range, including integers within such ranges and fractions of the values unless the context clearly indicates otherwise.

As used herein, “subject” means any animal, such as a mammal, to whom will be or has been treated by a method described herein. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc.

The phrase “pharmaceutically acceptable salt(s)” means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the application can form pharmaceutically acceptable salts with various amino acids. Suitable base salts include, but are not limited to, aluminum, calcium, lithium, magnesium, potassium, sodium, zinc, bismuth, and diethanolamine salts. For a review on pharmaceutically acceptable salts see Berge et al., 66 J. Pharm. Sci. 1-19 (1977), incorporated herein by reference.

As used herein, the term “alkyl” means a saturated, monovalent, unbranched or branched hydrocarbon chain. An alkyl group can be unsubstituted or substituted with one or more suitable substituents. Examples of alkyl groups include, but are not limited to, methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When numbers appear in a subscript after the symbol “C”, the subscript defines with more specificity the number of carbon atoms which that particular alkyl can contain. For example, “C₁ to C₁₀ alkyl” or “C₁₋₁₀ alkyl” is intended to include alkyl groups having 1, 2, 3, 4, 5, 6, 7, 8, 9, and 10 carbon atoms. Additionally, for example, “C₁ to C₄ alkyl” or “C₁₋₄ alkyl” denotes an alkyl having 1, 2, 3, or 4 carbon atoms.

The term “cycloalkyl” refers to any stable monocyclic or polycyclic saturated hydrocarbon ring system. A cycloalkyl group can be unsubstituted or substituted with one or more suitable substituents. A cycloalkyl group can have a specified number of carbon atoms. For example, “C₃ to C₆ cycloalkyl” or “C₃₋₆ cycloalkyl” includes cycloalkyl groups having 3, 4, 5, or 6 ring carbon atoms, i.e., cyclopropyl, cyclobutyl, cyclopentyl, and cyclohexyl. Polycyclic cycloalkyls include bridged, fused, and spiro ring structures in which all ring atoms are carbon atoms. A “spiro ring” is a polycyclic ring system in which two rings share one carbon atom, referred to as the “spiro atom,” which is typically a quaternary carbon atom. A “fused ring” is a polycyclic ring system in which two rings share two adjacent atoms, referred to as “bridgehead atoms,” i.e., the two rings share one covalent bond such that the bridgehead atoms are directly connected. A “bridged ring” is a polycyclic ring system in which two rings share three or more atoms separating the bridgehead atoms by a bridge containing at least one atom. Examples of cycloalkyl groups include, but are not limited to, cyclopropyl, cyclobutyl, cyclopentyl, cyclohexyl, cycloheptyl, cyclooctyl, etc.

The term “aryl” as used herein is a group that contains any carbon-based aromatic group including, but not limited to, phenyl, naphthyl, anthracenyl, phenanthranyl, and the like. Aryl moieties are well known and described, for example, in Lewis, R. J., ed., Hawley's Condensed Chemical Dictionary, 13th Edition, John Wiley & Sons, Inc., New York (1997). An aryl group can be substituted or unsubstituted with one or more suitable substituents. An aryl group can comprise a single ring structure (i.e., monocyclic) or multiple ring structures (i.e., polycyclic, e.g., bicyclic or tricyclic). For example, an aryl group can be a monocyclic aryl group, e.g., phenyl.

The term “heterocyclyl” includes stable monocyclic and polycyclic hydrocarbons that contain at least one heteroatom ring member, such as sulfur, oxygen, or nitrogen, wherein the ring structure is saturated or partially unsaturated, provided the ring system is not fully aromatic. A heterocyclyl group can be unsubstituted, or substituted with one or more suitable substituents at any one or more of the carbon atom(s) and/or nitrogen heteroatom(s) of the heterocyclyl. A heterocyclyl can comprise a single ring structure (i.e., monocyclic) or multiple ring structures (i.e., polycyclic, e.g., bicyclic). Polycyclic heterocyclyls include bridged, fused, and spiro ring structures in which at least one ring atom of at least one of the rings of the polycyclic ring system is a heteroatom, for instance oxygen, nitrogen, or sulfur, wherein bridged, fused, and spiro rings are as defined above. A heterocyclyl ring can be attached to the parent molecule at any suitable heteroatom (typically nitrogen) or carbon atom of the ring. The term “4- to 9-membered monocyclic or bicyclic heterocyclyl” includes any four, five, six, seven, eight, or nine membered monocyclic or bicyclic ring structure containing at least one heteroatom ring member selected from oxygen, nitrogen, and sulfur, or independently selected from oxygen and nitrogen, optionally containing one to three additional heteroatoms independently selected from oxygen, nitrogen, and sulfur, or independently selected from oxygen and nitrogen, wherein the ring structure is saturated or partially unsaturated, provided the ring structure is not fully aromatic.

In certain embodiments, the term “heterocyclyl” refers to 4-, 5-, 6-, or 7-membered monocyclic groups and 6-, 7-, 8-, or 9-membered bicyclic groups which have at least one heteroatom (O, S, or N) in at least one of the rings, wherein the heteroatom-containing ring(s) typically has 1, 2, or 3 heteroatoms, such as 1 or 2 heteroatoms, independently selected from O, S, and/or N, or independently selected from O and N. Examples of monocyclic heterocyclyl groups include, but are not limited to azetidinyl, oxetanyl, tetrahydrofuranyl, pyrrolidinyl, imidazolidinyl, pyrazolidinyl, oxazolidinyl, isoxazolidinyl, thiazolidinyl, isothiazolidinyl, dioxolanyl, dithiolanyl, piperidinyl, piperazinyl, dioxanyl, morpholinyl, azepanyl, oxepanyl, oxazepanyl (e.g., 1,4-oxazepanyl, 1,2-oxazepanyl) and the like. Examples of bicyclic heterocyclyl groups include, but are not limited to, 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, and 5-azaspiro[2.3]hexanyl and the like.

As used herein, the term “heteroaryl” includes stable monocyclic and polycyclic aromatic hydrocarbons that contain at least one heteroatom ring member such as sulfur, oxygen, or nitrogen. A heteroaryl group can be unsubstituted or substituted with one or more suitable substituents. A heteroaryl can comprise a single ring structure (i.e., monocyclic) or multiple ring structures (i.e., polycyclic, e.g., bicyclic or tricyclic). Each ring of a heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring, but the other fused ring or rings can be aromatic or non-aromatic. For example, for a bicyclic heteroaryl, the fused rings completing the bicyclic group can contain only carbon atoms and can be saturated, partially saturated, or unsaturated. A heteroaryl can be attached to the parent molecule at any available nitrogen or carbon atom of any ring of the heteroaryl group. In some embodiments, the term “heteroaryl” refers to 5- or 6-membered monocyclic groups and 9- or 10-membered bicyclic groups which have at least one heteroatom (O, S, or N) in at least one of the rings, wherein the heteroatom-containing ring typically has 1, 2, or 3 heteroatoms, such as 1 or 2 heteroatoms, selected from O, S, and/or N. A heteroaryl group can be unsubstituted, or substituted with one or more suitable substituents at any one or more of the carbon atom(s) and/or nitrogen heteroatom(s) of the heteroaryl. The nitrogen and sulfur heteroatom(s) of a heteroaryl can optionally be oxidized (i.e., N→O and S(O)r, wherein r is 0, 1 or 2).

Exemplary monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl. Exemplary bicyclic heteroaryl groups include, but are not limited to, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridinyl, furopyridinyl, dihydroisoindolyl, and tetrahydroquinolinyl.

The term “alkoxy” as used herein refers to an —O-alkyl group, wherein alkyl is as defined above. An alkoxy group is attached to the parent molecule through a bond to an oxygen atom. An alkoxy group can have a specified number of carbon atoms. For example, “C₁ to C₁₀ alkoxy” or “C₁₋₁₀ alkoxy” is intended to include alkoxy groups having 1, 2, 3, 4, 5, 6, 7, 8, 9, and 10 carbon atoms. Additionally, for example, “C₁ to C₄ alkoxy” or “C₁₋₄ alkoxy” denotes an alkoxy having 1, 2, 3, or 4 carbon atoms. Examples of alkoxy include, but are not limited to, methoxy, ethoxy, propoxy (e.g., n-propoxy, isopropoxy), butoxy (e.g., n-butoxy, isobutoxy, tert-butoxy), pentyloxy (e.g., n-pentyloxy, isopentyloxy, neopentyloxy), etc. An alkoxy group can be unsubstituted or substituted with one or more suitable substituents. Similarly, “alkylthio” or “thioalkoxy” represents an alkyl group as defined above attached to the parent molecule through a bond to a sulfur atom, for example, —S-methyl, —S-ethyl, etc. Representative examples of alkylthio include, but are not limited to, —SCH₃, —SCH₂CH₃, etc.

As used herein, the term “halogen” means fluorine, chlorine, bromine, or iodine. Correspondingly, the term “halo” means fluoro, chloro, bromo, and iodo.

“Haloalkyl” is intended to include both branched and straight-chain saturated aliphatic hydrocarbon radicals substituted with one or more halogen atoms. “Fluorinated alkyl” or “fluoroalkyl” in particular refers to any alkyl group as defined above substituted with at least one fluoro atom, e.g., one to three fluoro atoms, such as one, two, or three fluoroatoms. Examples of haloalkyl include, but are not limited to, fluoromethyl, difluoromethyl, trifluoromethyl, chloromethyl, dichloromethyl, trichloromethyl, pentafluoroethyl, pentachloroethyl, 2,2,2-trifluoroethyl, heptafluoropropyl, and heptachloropropyl. Suitable examples of fluoroalkyl in particular include, but are not limited to, —CF₃, —CHF₂, —CH₂F, —CH₂CF₃, —CF₂CF₃, and the like.

The terms “hydroxy” and “hydroxyl” can be used interchangeably, and refer to —OH.

The term “carboxy” refers to —COOH.

The term “ester” refers to —COOR, wherein R is alkyl as defined above.

The term “cyano” refers to —CN.

The term “oxo” refers to a double bonded oxygen group, i.e., a substituent group of the formula ═O.

The term “keto” refers to —C(O)R, wherein R is alkyl as defined above.

The term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

The term “aminoalkyl” as used herein is intended to include both branched and straight-chain saturated aliphatic hydrocarbon groups substituted with one or more amino groups. For example, “C₁₋₄ aminoalkyl” is intended to include alkyl groups having 1, 2, 3, or 4 carbon atoms substituted with one or more amino groups. Aminoalkyl groups are attached to the parent molecule through a bond to a carbon atom of the alkyl moiety of the aminoalkyl group. Representative examples of aminoalkyl groups include, but are not limited to, —CH₂NH₂, —CH₂CH₂NH₂, and —CH₂CH(NH₂)CH₃.

As used herein, “amido” refers to —C(O)N(R)₂, wherein each R is independently an alkyl group (including both branched and straight-chain alkyl groups) or a hydrogen atom. Examples of amido groups include, but are not limited to, —C(O)NH₂, —C(O)NHCH₃, and —C(O)N(CH₃)₂.

The terms “hydroxylalkyl” and “hydroxyalkyl” are used interchangeably, and refer to a branched or straight-chain aliphatic hydrocarbon group substituted with one or more hydroxyl groups. Hydroxyalkyl groups are attached to the parent molecule through a bond to a carbon atom of the alkyl moiety of the hydroxyalkyl group. A hydroxyalkyl group can have a specified number of carbon atoms. For example, “C₁ to C₁₀ hydroxyalkyl” or “C₁₋₁₀ hydroxyalkyl” is intended to include hydroxyalkyl groups having 1, 2, 3, 4, 5, 6, 7, 8, 9, and 10 carbon atoms. Additionally, for example, “C₁ to C₄ hydroxylalkyl” or “C₁₋₄ hydroxyalkyl” denotes a hydroxyalkyl group having 1, 2, 3, or 4 carbon atoms. Examples of hydroxyalkyl include, but are not limited to, hydroxylmethyl (—CH₂OH), hydroxylethyl (—CH₂CH₂OH), etc.

In accordance with convention used in the art:

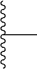

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

The term “substituted” as used herein with respect to any organic radical (e.g., alkyl, cycloalkyl, heteroaryl, aryl, heterocyclyl, etc.) means that at least one hydrogen atom is replaced with a non-hydrogen group, provided that all normal valencies are maintained and that the substitution results in a stable compound. When a particular group is “substituted,” that group can have one or more substituents, such as from one to five substituents, one to three substituents, or one to two substituents, independently selected from the list of substituents. The term “independently” when used in reference to substituents, means that when more than one of such substituents is possible, such substituents can be the same or different from each other. Examples of suitable substituents include, but are not limited to, alkyl, halo, haloalkyl, alkoxy, amido, hydroxy, hydroxyalkyl, amino, carboxyl, ester, oxo, cyano and the like.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

The terms “optional” or “optionally” mean that the event or circumstance described subsequently can, but need not, occur, and such a description includes the situation in which the event or circumstance does or does not occur. For example, “optionally substituted heterocyclyl” means that a substituent group can be, but need not be, present, and such a description includes the situation of the heterocyclyl group being substituted by a suitable substituent and the heterocyclyl group not being substituted by any substituent.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds, or otherwise indicated as having a particular configuration (e.g., R or S) around one or more atoms, contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or diastereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Thus, included within the scope of the application are the stereochemically pure isomeric forms of the compounds described herein (i.e., a single enantiomer or a single diastereomer) as well as mixtures thereof including their racemates. When a specific stereoisomer is identified, this means that the stereoisomer is substantially free, i.e., associated with less than 50%, less than 20%, less than 5%, and in particular less than 2% or less than 1% of the other stereoisomers. For example, when a compound is for instance specified as (R), this means that the compound is substantially free of the (S) isomer. Compounds described herein can be used as racemic mixtures, enantiomerically or diastereomerically enriched mixtures, or as enantiomerically or diastereomerically pure individual stereoisomers.

Certain examples contain chemical structures that comprise (*R) or (*S) terminology. When (*R) or (*S) is used in the name of a compound or in the chemical representation of the compound, it is intended to mean that the compound is a single isomer at that stereocenter, however absolute configuration of that stereocenter has not been established. Thus, a compound designated as (*R) refers to a compound that is a single isomer at that stereocenter with an absolute configuration of either (R) or (S). A compound designated as (*S) refers to a compound that is a single isomer at that stereocenter with an absolute configuration of either (R) or (S). In cases where the absolute stereochemistry has been established, the structures are named using (R) or (S). The use of the term (R, S) or “racemic” or “rac” in the name of the compound indicates that the compound is a racemate.

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diastereomeric salts with optically active acids or bases or by chiral chromatography. Pure stereoisomers can also be prepared synthetically from appropriate stereochemically pure starting materials, or by using stereoselective reactions.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the application.

Compounds described herein can exist in solvated and unsolvated forms. The term “solvate” means a physical association, e.g., by hydrogen bonding, of a compound of the application with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds of the application can form solvates with water (i.e., hydrates) or common organic solvents. Exemplary solvates include, but are not limited to, hydrates, ethanolates, methanolates, and isopropanolates. Methods of solvation are generally known in the art.

Also included within the scope of the application are all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds can generally be prepared by conventional techniques known to those skilled in the art or by processes analogous to those described herein, using an appropriate isotopically-labeled reagent in place of the non-labeled reagent otherwise employed.

As used herein, the name of a compound is intended to encompass all possible existing isomeric forms, including stereoisomers (e.g., enantiomers, diastereomers, racemate or racemic mixture, and any mixture thereof) of the compound.

### Compounds

In one general aspect, provided herein are compounds having a pyrazolopyridine core and which are inhibitors of salt inducible kinases (SIKs).

In an embodiment, provided is a compound of formula (II):

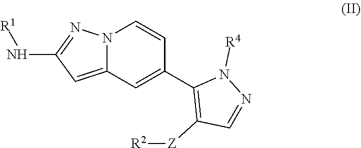

or a stereoisomer, tautomer, pharmaceutically acceptable salt or solvate thereof, wherein:
Z is —O—(CH₂)ₙ—, provided that O is directly bonded to the pyrazolyl;
n is 0 or 1;
R⁴ is —CH₃, —CH₂CH₃, —CHF₂, —CD₃, cyclopropyl or benzyl;
R¹ is —C(O)R⁵, phenyl, or a 5- to 6-membered heteroaryl having 1-2 nitrogen heteroatoms, or

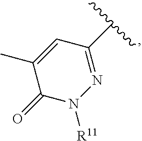

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl, wherein the 5- to 6-membered heteroaryl is optionally fused to a ring selected from the group consisting of benzene, cyclopentane, tetrahydropyran, and a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, —CN, and phenyl;
R² is:
   a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH₃, —OCF₃, —CN, cyclopropyl, oxetanyl, tetrahydrofuranyl, pyrazolyl, and phenyl,
   a C₁₋₅ alkyl, wherein the C₁₋₅ alkyl is optionally substituted with one or more substituents independently selected from the group consisting of —OH, —N(R¹⁰)₂, C₁ fluoroalkyl, —COOR⁸, —CH₃, C₃-C₆ cycloalkyl, phenyl, pyridyl, 3-bicyclo[1.1.1]pentanyl, and —CN, wherein the C₃-C₆ cycloalkyl is optionally substituted with one or more —CH₃, or —NHCH₃, or
   a C₃-C₆ cycloalkyl, wherein the C₃-C₆ cycloalkyl is optionally substituted with one or more —OH;
each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F;
each R⁹ is independently C₁ fluoroalkyl or —CH₃;
each R¹⁰ is independently hydrogen, cyclopropyl, cyclopentyl, or —CH₃;
each R¹¹ is independently hydrogen, C₁₋₅ alkyl, or —CH₂-cyclopropyl; and
m is 1 or 2.

In some embodiments, provided is a compound of formula (II), or a stereoisomer, tautomer, pharmaceutically acceptable salt or solvate thereof, wherein R⁴ is —CH₃.

In some embodiments, provided is a compound of formula (II), or a stereoisomer, tautomer, pharmaceutically acceptable salt or solvate thereof, wherein R⁴ is —CH₂CH₃.

In some embodiments, provided is a compound of formula (II), or a stereoisomer, tautomer, pharmaceutically acceptable salt or solvate thereof, wherein R⁴ is —CHF₂.

In some embodiments, provided is a compound of formula (II), or a stereoisomer, tautomer, pharmaceutically acceptable salt or solvate thereof, wherein R⁴ is —CD₃.

In some embodiments, provided is a compound of formula (II), or a stereoisomer, tautomer, pharmaceutically acceptable salt or solvate thereof, wherein R⁴ is —CH₂-cyclopropyl.

In some embodiments, provided is a compound of formula (II), or a stereoisomer, tautomer, pharmaceutically acceptable salt or solvate thereof, wherein R⁴ is benzyl.

In some embodiments, provided is a compound of formula (II), or a stereoisomer, tautomer, pharmaceutically acceptable salt or solvate thereof, wherein n is 0 such that Z is —O—.

In some embodiments, provided is a compound of formula (II), or a stereoisomer, tautomer, pharmaceutically acceptable salt or solvate thereof, wherein n is 1 such that Z is —O—CH₂—, provided that O is directly bonded to pyrazolyl.

In some embodiments, provided is a compound of formula (II), or a stereoisomer, tautomer, pharmaceutically acceptable salt or solvate thereof, wherein R¹ is phenyl.

In some embodiments, provided is a compound of formula (II), or a stereoisomer, tautomer, pharmaceutically acceptable salt or solvate thereof, wherein R¹ is —C(O)R⁵, and R⁵ is cyclopropyl optionally substituted with one or more substituents independently selected from the group consisting of —F, —CH₃, C₁ fluoroalkyl, —CN, and phenyl.

In some embodiments, provided is a compound of formula (II), or a stereoisomer, tautomer, pharmaceutically acceptable salt or solvate thereof, wherein R¹ is —C(O)R⁵, and R⁵ is cyclopropyl.

In some embodiments, provided is a compound of formula (II), or a stereoisomer, tautomer, pharmaceutically acceptable salt or solvate thereof, wherein R¹ is —C(O)R⁵, and R⁵ is cyclopropyl substituted with 1-2 fluoro atoms.

In some embodiments, provided is a compound of formula (II), or a stereoisomer, tautomer, pharmaceutically acceptable salt or solvate thereof, wherein R¹ is —C(O)R⁵, and R⁵ is cyclopropyl substituted with —CH₃.

In some embodiments, provided is a compound of formula (II), or a stereoisomer, tautomer, pharmaceutically acceptable salt or solvate thereof, wherein R¹ is —C(O)R⁵, and R⁵ is cyclopropyl substituted with —CF₃.

In some embodiments, provided is a compound of formula (II), or a stereoisomer, tautomer, pharmaceutically acceptable salt or solvate thereof, wherein R¹ is —C(O)R⁵, and R⁵ is cyclopropyl substituted with —CN.

In some embodiments, provided is a compound of formula (II), or a stereoisomer, tautomer, pharmaceutically acceptable salt or solvate thereof, wherein R¹ is —C(O)R⁵, and R⁵ is cyclopropyl substituted with phenyl.

In some embodiments, provided is a compound of formula (II), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl, wherein the 5- to 6-membered heteroaryl is optionally fused to a ring selected from the group consisting of benzene, cyclopentane, tetrahydropyran, and a 5- to 6-membered heteroaryl having 1-2 nitrogen heteroatoms, wherein the 5- to 6-membered heteroaryl is optionally substituted with one or more —CH₃; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is

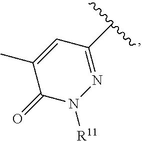

wherein each R¹¹ is independently hydrogen, —CH₃, —CH₂CH₃, or —CH₂-cyclopropyl.

In some embodiments, provided is a compound of formula (II), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl, wherein the 5- to 6-membered heteroaryl is fused to a ring selected from the group consisting of benzene, cyclopentane, tetrahydropyran, and a 5- to 6-membered heteroaryl having 1-2 nitrogen heteroatoms, wherein the 5- to 6-membered heteroaryl is optionally substituted with one or more —CH₃; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is 7,8-dihydro-5H-pyrano[4,3-b]pyridine, 6,7-dihydro-5H-cyclopenta[b]pyridine, pyrrolo[2,3-b]pyridine, imidazo[1,2-b]pyridazine, and pyrazolo[1,5-a]pyrimidine.

In some embodiments, provided is a compound of formula (II), or a stereoisomer, tautomer, pharmaceutically acceptable salt or solvate thereof, wherein R¹ is pyrazolyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is pyrazolyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, cyclopropyl, —CH₂-cyclopropyl, —OCHF₂, —COOCH₃, pyridinyl, and

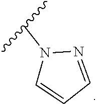 [SMILES: *n1cccn1]

In some embodiments, provided is a compound of formula (II), or a stereoisomer, tautomer, pharmaceutically acceptable salt or solvate thereof, wherein R¹ is pyridinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is pyridinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, cyclopropyl, —CH₂-cyclopropyl, —OCHF₂, —COOCH₃, pyridinyl, and

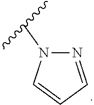 [SMILES: *n1cccn1]

In some embodiments, provided is a compound of formula (II), or a stereoisomer, tautomer, pharmaceutically acceptable salt or solvate thereof, wherein R¹ is pyrazinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is pyrazinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, cyclopropyl, —CH₂-cyclopropyl, —OCHF₂, —COOCH₃, pyridinyl, and

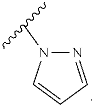 [SMILES: *n1cccn1]

In some embodiments, provided is a compound of formula (II), or a stereoisomer, tautomer, pharmaceutically acceptable salt or solvate thereof, wherein R¹ is pyrimidinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is pyrimidinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, cyclopropyl, —CH₂— cyclopropyl, —OCHF₂, —COOCH₃, pyridinyl, and

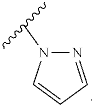 [SMILES: *n1cccn1]

In some embodiments, provided is a compound of formula (II), or a stereoisomer, tautomer, pharmaceutically acceptable salt or solvate thereof, wherein R¹ is pyridazinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is pyridazinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, cyclopropyl, —CH₂— cyclopropyl, —OCHF₂, —COOCH₃, pyridinyl, and

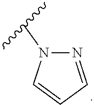 [SMILES: *n1cccn1]

In some embodiments, provided is a compound of formula (II), or a stereoisomer, tautomer, pharmaceutically acceptable salt or solvate thereof, wherein R¹ is phenyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F; each R⁹ is independently C₁ fluoroalkyl or —CH₃; and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R¹ is phenyl optionally substituted with —COOCH₃, —C(O)N(H)CH₂CH₃, —C(O)N(H)CH₂CF₃, —OCH₃, and

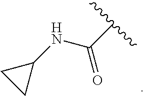 [SMILES: *C(=O)NC1CC1]

In some embodiments, provided is a compound of formula (II), or a pharmaceutically acceptable salt thereof, wherein R¹ is:

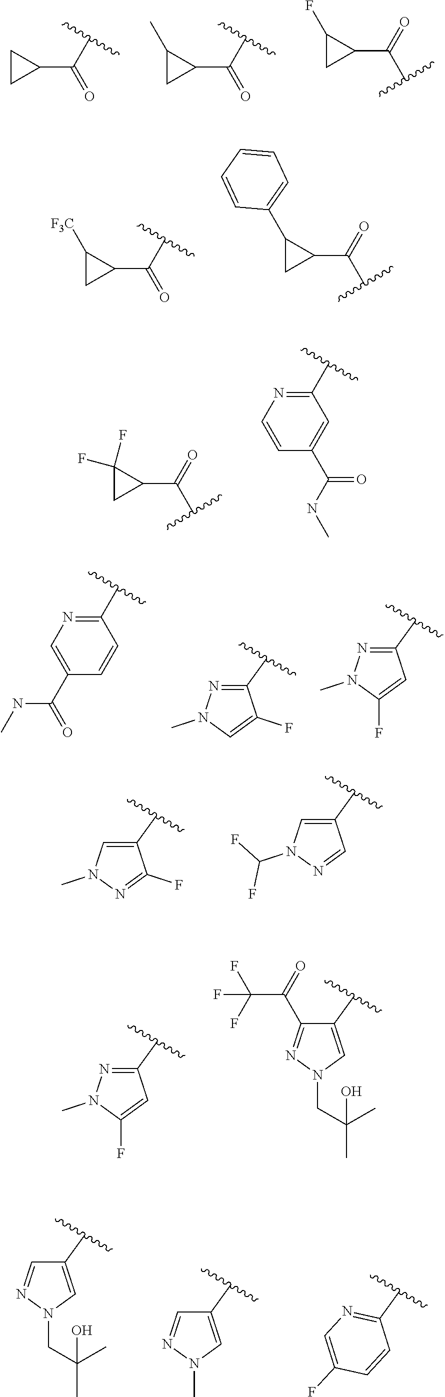

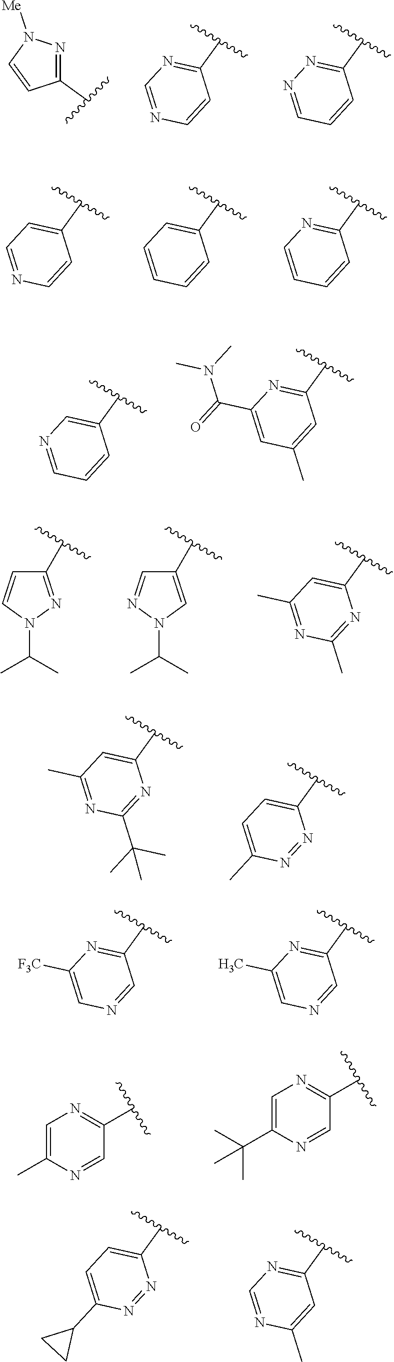

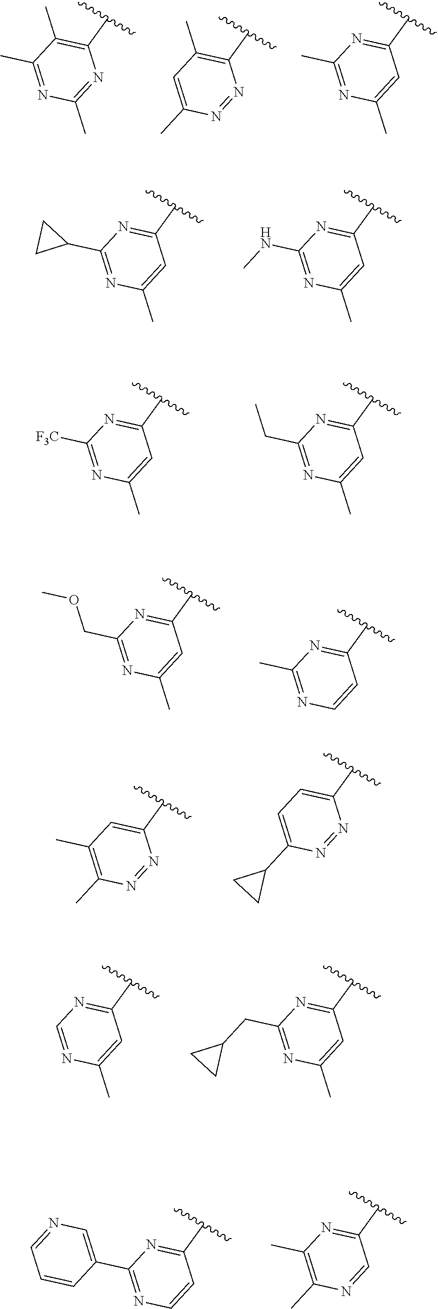

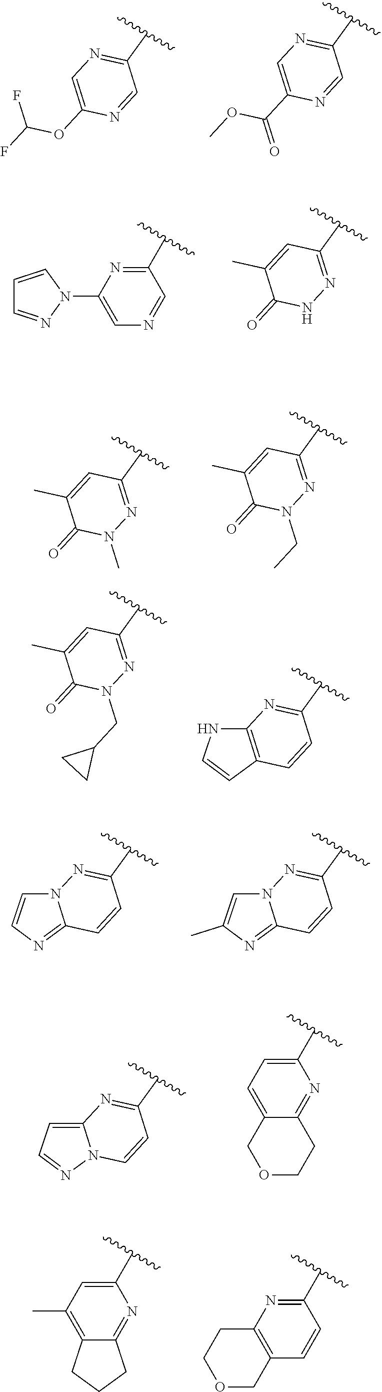

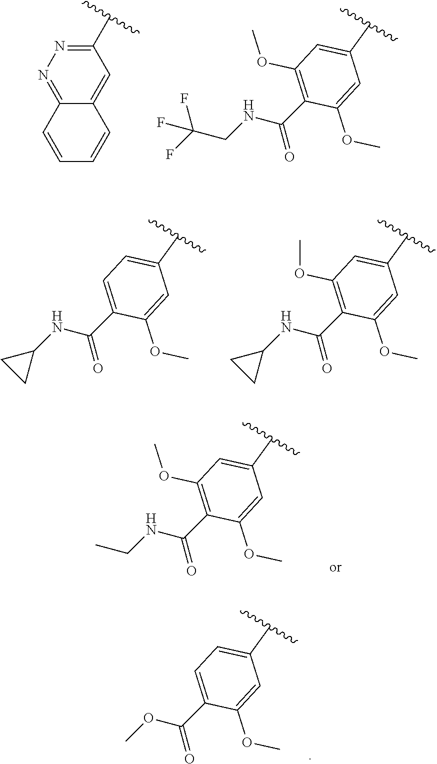

In some embodiments, provided is a compound of formula (II), or a stereoisomer, tautomer, pharmaceutically acceptable salt or solvate thereof, wherein R⁸ is a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH₃, —OCF₃, —CN, cyclopropyl, oxetanyl, tetrahydrofuranyl, pyrazolyl, and phenyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F, each R¹⁰ is independently hydrogen, cyclopropyl, cyclopentyl, or —CH₃, and m is 1 or 2.

In some embodiments, provided is a compound of formula (II), or a stereoisomer, tautomer, pharmaceutically acceptable salt or solvate thereof, wherein R² is a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), phenyl, pyridinyl, —CH₂CH(CH₃)₂, —CD₃, —CD₂CD₃, —CH₂CH₂F, —CH₂CH₂OF₃, —CH₂CH(OH)CH₃, —CH₂C(OH)(CH₃)₂, —CH₂C(OCH₃)(CH₃)₂, —CH₂CH₂—OCH₃, —CH₂C(O)CH₃, —CH₂CH₂—CN, —CH₂C(CN)(CH₃)₂, —CH₂— cyclopropyl, —CH₂-phenyl,

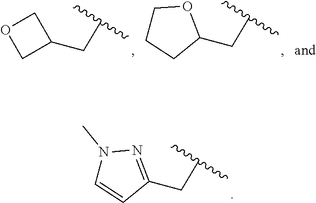

In some embodiments, provided is a compound of formula (II), or a stereoisomer, tautomer, pharmaceutically acceptable salt or solvate thereof, wherein R² is a 4- to 7-membered monocyclic heterocyclyl having 1 to 2 heteroatoms independently selected from oxygen and nitrogen, wherein the 4- to 7-membered monocyclic heterocyclyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH3, —OCF3, —CN, cyclopropyl, oxetanyl, tetrahydrofuranyl, pyrazolyl, and phenyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F, each R¹⁰ is independently hydrogen, cyclopropyl, cyclopentyl, or —CH₃, and m is 1 or 2.

In some embodiments, provided is a compound of formula (II), or a stereoisomer, tautomer, pharmaceutically acceptable salt or solvate thereof, wherein R² is a 4- to 7-membered monocyclic heterocyclyl having 1 to 2 heteroatoms independently selected from oxygen and nitrogen, wherein the 4- to 7-membered monocyclic heterocyclyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), phenyl, pyridinyl, —CH₂CH(CH₃)₂, —CD₃, —CD₂CD₃, —CH₂CH₂F, —CH₂CH₂OF₃, —CH₂CH(OH)CH₃, —CH₂C(OH)(CH₃)₂, —CH₂C(OCH₃)(CH₃)₂, —CH₂CH₂—OCH₃, —CH₂C(O)CH₃, —CH₂CH₂—CN, —CH₂C(CN)(CH₃)₂, —CH₂-cyclopropyl, —CH₂— phenyl,

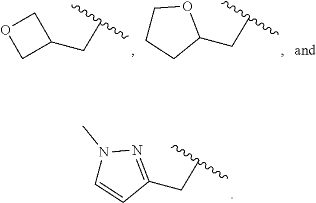

In some embodiments, provided is a compound of formula (II), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, and oxetanyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH3, —OCF3, —CN, cyclopropyl, oxetanyl, tetrahydrofuranyl, pyrazolyl, and phenyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F, each R¹⁰ is independently hydrogen, cyclopropyl, cyclopentyl, or —CH₃, and m is 1 or 2.

In some embodiments, provided is a compound of formula (II), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, and oxetanyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), phenyl, pyridinyl, —CH₂CH(CH₃)₂, —CD₃, —CD₂CD₃, —CH₂CH₂F, —CH₂CH₂OF₃, —CH₂CH(OH)CH₃, —CH₂C(OH)(CH₃)₂, —CH₂C(OCH₃)(CH₃)₂, —CH₂CH₂—OCH₃, —CH₂C(O)CH₃, —CH₂CH₂—CN, —CH₂C(CN)(CH₃)₂, —CH₂-cyclopropyl, —CH₂-phenyl,

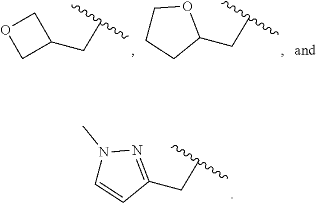

In some embodiments, provided is a compound of formula (II), or a stereoisomer, tautomer, pharmaceutically acceptable salt or solvate thereof, wherein R² is a 4- to 7-membered monocyclic heterocyclyl selected from the group consisting of:

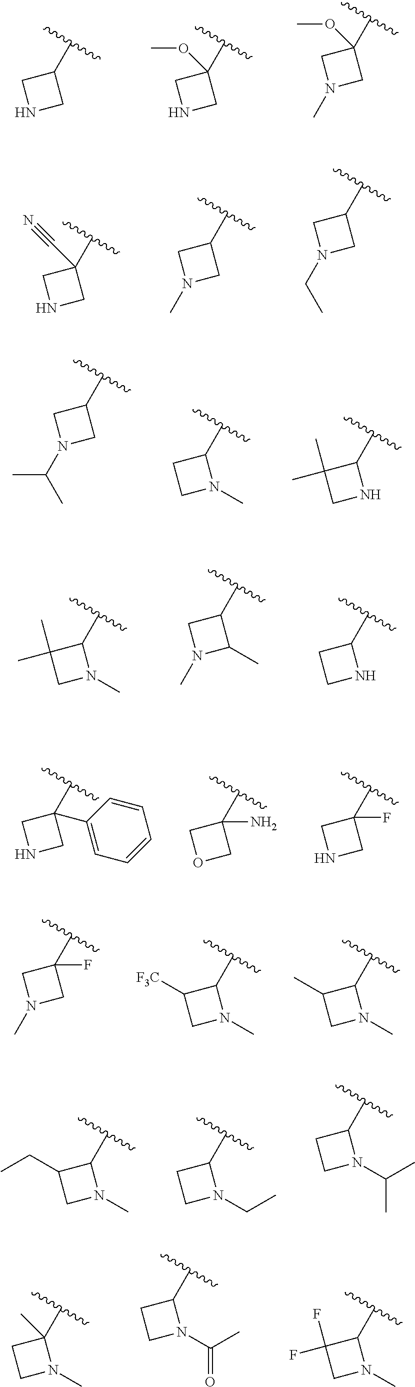

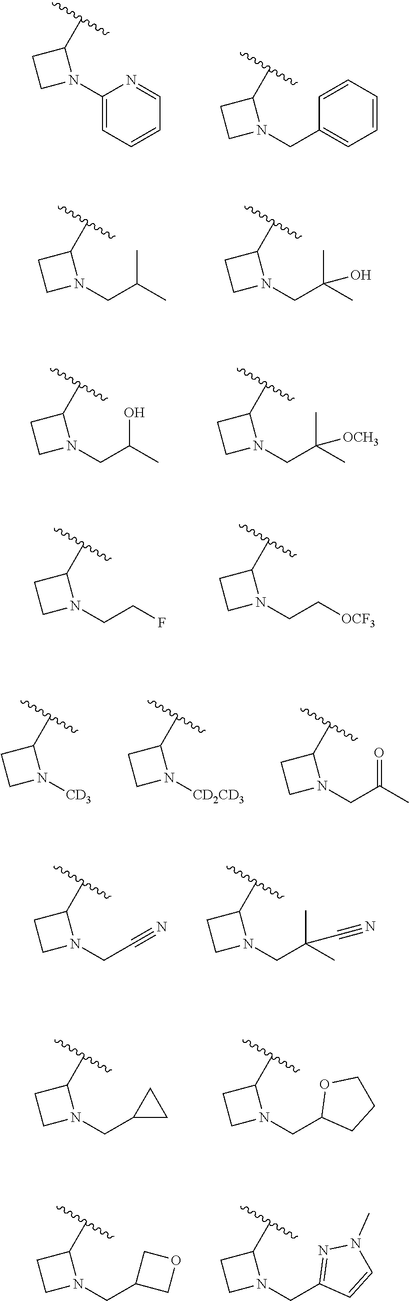

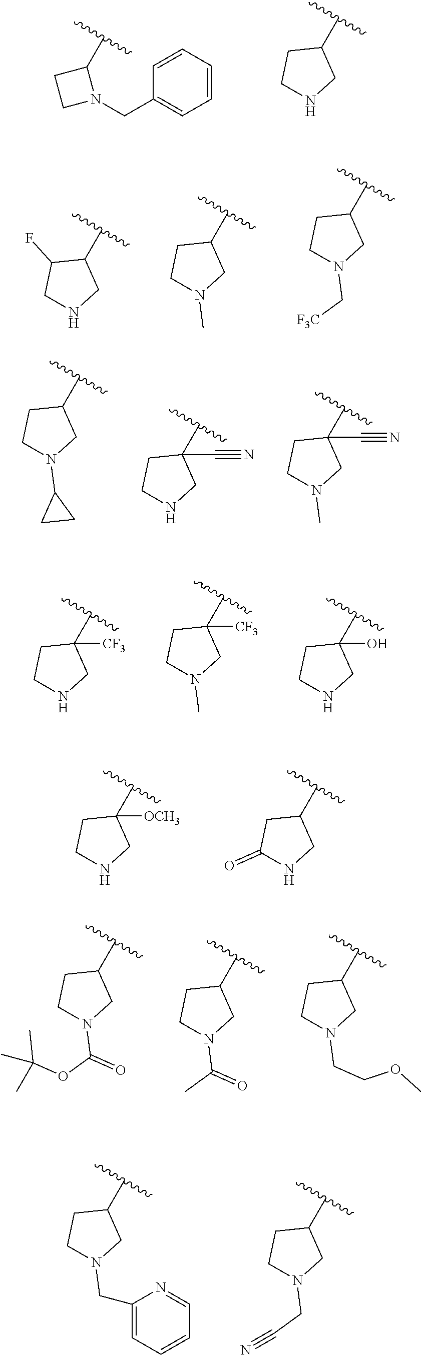

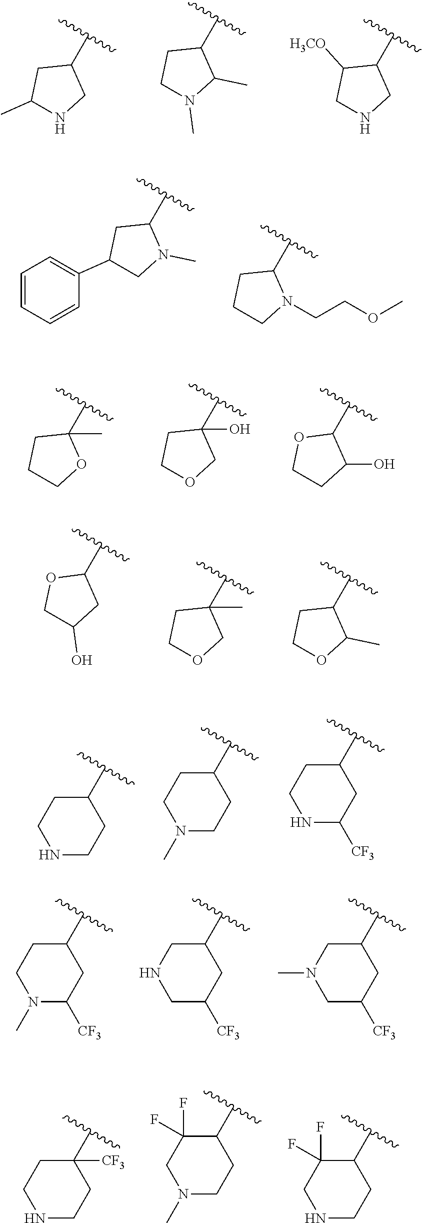

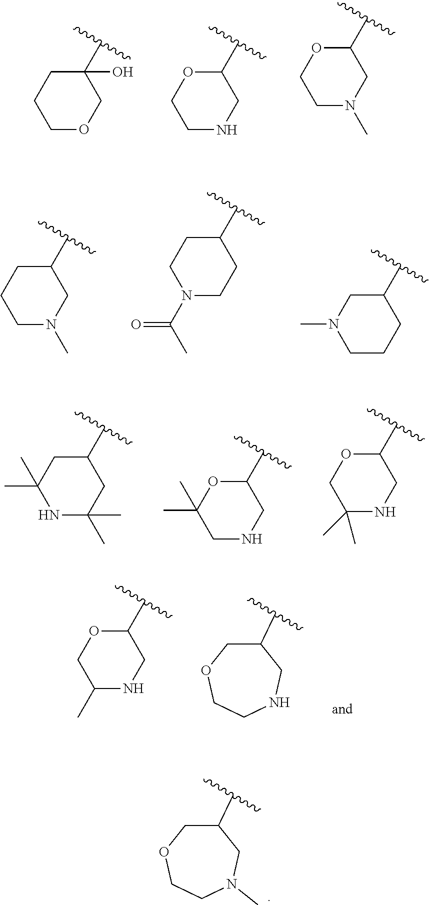

In some embodiments, provided is a compound of formula (II), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of C₁₋₄ alkyl, C₁₋₂ fluoroalkyl, —CD₃, —CD₂CD₃, —F, —OH, —OCH₃, oxo, cyclopropyl, —COOR⁸, —C(O)CH₃, —CH₂C(O)CH₃, —N(R¹⁰)₂, —(CH₂)ₘOCH₃, —CH₂CN, —CH₂(C₅H₄N), pyridinyl, and phenyl, wherein the C₁-C₄ alkyl is optionally substituted with one or more substituents independently selected from the group consisting of —OH, —OCH3, —OCF3, —CN, cyclopropyl, oxetanyl, tetrahydrofuranyl, pyrazolyl, and phenyl; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F, each R¹⁰ is independently hydrogen, cyclopropyl, cyclopentyl, or —CH₃, and m is 1 or 2.

In some embodiments, provided is a compound of formula (II), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), phenyl, pyridinyl, —CH₂CH(CH₃)₂, —CD₃, —CD₂CD₃, —CH₂CH₂F, —CH₂CH₂OF₃, —CH₂CH(OH)CH₃, —CH₂C(OH)(CH₃)₂, —CH₂C(OCH₃)(CH₃)₂, —CH₂CH₂—OCH₃, —CH₂C(O)CH₃, —CH₂CH₂—CN, —CH₂C(CN)(CH₃)₂, —CH₂-cyclopropyl, —CH₂-phenyl,

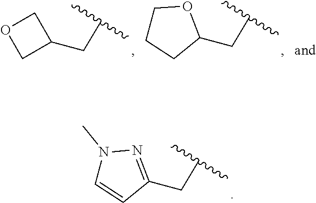

In some embodiments, provided is a compound of formula (II), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl is optionally substituted with one or more substituents independently selected from the group consisting of C₁₋₃ alkyl, C₁₋₂ fluoroalkyl, —F, —OH, —OCH₃, oxo, —CN, cyclopropyl, —COOR⁸, —C(O)CH₃, —N(R¹⁰)₂, —(CH₂)ₘOCH₃, —CH₂CN, —CH₂(C₅H₄N), and phenyl, wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (II), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl is optionally substituted with one or more substituents independently selected from the group consisting of —CH₃, —OCH₃, —CH₂CH₃, —CH(CH₃)₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), phenyl, pyridinyl, —CH₂CH(CH₃)₂, —CD₃, —CD₂CD₃, —CH₂CH₂F, —CH₂CH₂OF₃, —CH₂CH(OH)CH₃, —CH₂C(OH)(CH₃)₂, —CH₂C(OCH₃)(CH₃)₂, —CH₂CH₂—OCH₃, —CH₂C(O)CH₃, —CH₂CH₂—CN, —CH₂C(CN)(CH₃)₂, —CH₂-cyclopropyl, —CH₂-phenyl,

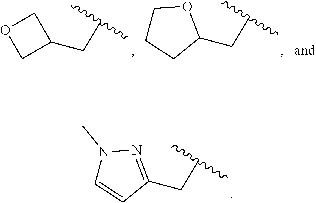

In some embodiments, provided is a compound of formula (II), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl is optionally substituted with one or more substituents independently selected from the group consisting of —CH₃, —CH₂CH₃, —COOC(CH₃)₃, and oxo.

In some embodiments, provided is a compound of formula (II), or a stereoisomer, tautomer, pharmaceutically acceptable salt or solvate thereof, wherein R² is a 6- to 9-membered bicyclic heterocyclyl selected from the group consisting of:

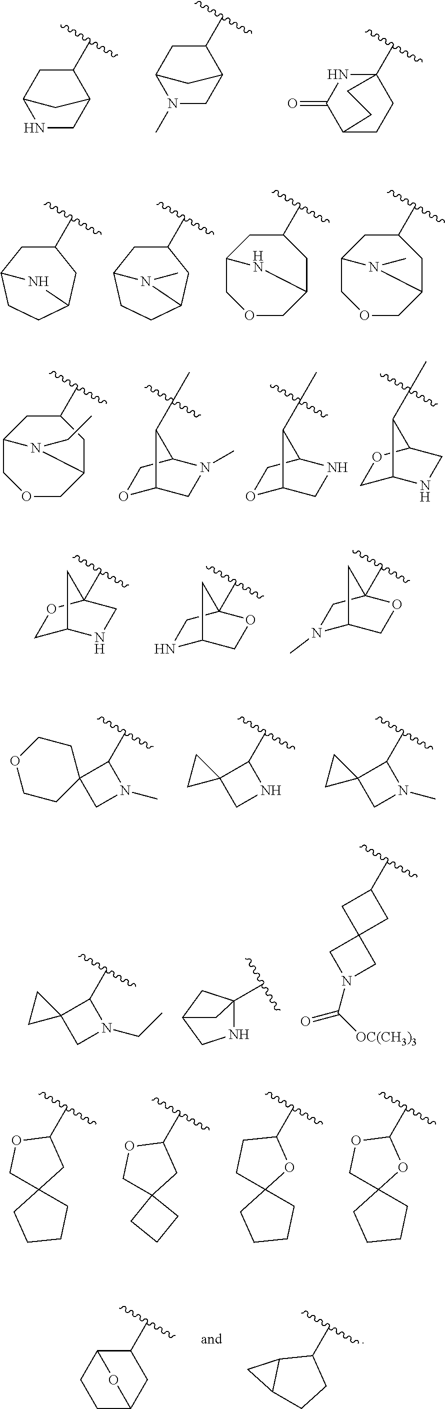

In some embodiments, provided is a compound of formula (II), or a stereoisomer, tautomer, pharmaceutically acceptable salt or solvate thereof, wherein R² is a C₁₋₅ alkyl optionally substituted with one or more substituents independently selected from the group consisting of —OH, —N(R¹⁰)₂, C₁ fluoroalkyl, —COOR⁸, —CH₃, C₃-C₆ cycloalkyl, phenyl, pyridyl, 3-bicyclo[1.1.1]pentanyl, and —CN, wherein the C₃-C₆ cycloalkyl is optionally substituted with one or more —CH₃, or —NHCH₃; wherein each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F, and each R¹⁰ is independently hydrogen, cyclopropyl, cyclopentyl, or —CH₃.

In some embodiments, provided is a compound of formula (II), or a stereoisomer, tautomer, pharmaceutically acceptable salt or solvate thereof, wherein R² is selected from the group consisting of —CH₂CH₂N(CH₃)₂, —C(OH)(CH₃)CF₃, —C(CH₃)₂OH, —CH(OH)CH₃, —CH(OH)CF₃, —C(OH)(CF₃)₂, —CH(OH)CH₃, —CH(NH₂)C(CH₃)₃, —CH₂C(O)OC(CH₃)₃, and —C(CH₃)(NH₂)CF₃, —CH₂CH(OH)(C₆H₅), —CH₂C(OH)(CH₃)CF₃, —CH₂C(OH)(CH₃)(C₃H₅), —CH₂CH₂(C₅H₄N), —CH₂CH(OH)(C₆H₁₁), —CH₂C(OH)(CH₃)₂, —CH₂CH(NH₂)(C₆H₅), —CH₂C(OH)(CF₃)₂,

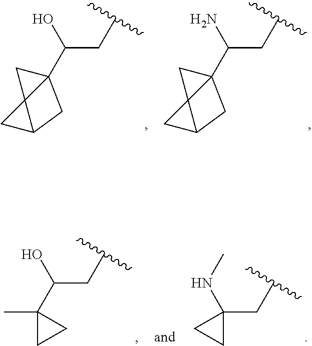

In some embodiments, provided is a compound of formula (II), or a stereoisomer, tautomer, pharmaceutically acceptable salt or solvate thereof, wherein R² is a C₃-C₆ cycloalkyl, wherein the C₃-C₆ cycloalkyl is optionally substituted with one or more —OH.

In some embodiments, provided is a compound of formula (II), or a stereoisomer, tautomer, pharmaceutically acceptable salt or solvate thereof, wherein R² is a cyclobutyl, wherein the cyclobutyl is optionally substituted with one or more —OH.

In some embodiments, provided is a compound of formula (II), or a stereoisomer, tautomer, pharmaceutically acceptable salt or solvate thereof, wherein each R⁸ is independently hydrogen, —CH₃, —C(CH₃)₃, cyclopropyl, —CH₂CH₃, —CH₂CF₃, —CH₂C(CN)(CH₃)₂, —CH₂CH₂—CN, —CH₂C(OH)(CH₃)₂, or —CH₂CH₂—OCH₃.

In some embodiments, provided is a compound of formula (II), or a stereoisomer, tautomer, pharmaceutically acceptable salt or solvate thereof, wherein R⁹ is —CF₃.

In some embodiments, provided is a compound of formula (II), or a stereoisomer, tautomer, pharmaceutically acceptable salt or solvate thereof, wherein R⁹ is —CH₃.

In some embodiments, provided is a compound of formula (II), or a stereoisomer, tautomer, pharmaceutically acceptable salt or solvate thereof, wherein R¹¹ is hydrogen, —CH₃, —CH₂CH₃, and —CH₂(C₃H₅).

All possible combinations of the above-indicated embodiments of compounds of formula (II) and their tautomers, stereoisomers, pharmaceutically acceptable salts and solvates are considered to be embraced within the scope of this application.

In another embodiment, provided is a compound of formula (III):

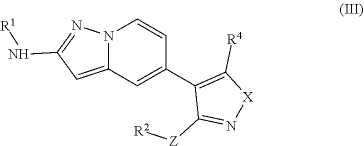

or a stereoisomer, tautomer, pharmaceutically acceptable salt or solvate thereof, wherein:
X is O or S;
Z is —O—(CH₂)ₙ—, provided that O is directly bonded to the isoxazolyl or isothiazolyl;
n is 0 or 1;
R⁴ is —CH₃ or —CH₂CH₃;
R¹ is —C(O)R⁵, phenyl, a 5- to 6-membered heteroaryl having 1-2 nitrogen heteroatoms, or

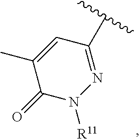

wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, and —OCH₃, wherein the 5- to 6-membered heteroaryl is optionally fused to a 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R² is:
   a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl, or
   a C₁₋₅ alkyl, wherein the C₁₋₅ alkyl is optionally substituted with one or more substituents independently selected from the group consisting of —OH, —N(R¹⁰)₂, C₁ fluoroalkyl, —COOR₈, —CH₃, C₃-C₆ cycloalkyl, phenyl, pyridyl, and —CN, wherein the phenyl is optionally substituted with one or more —F, or
   a C₃-C₆ cycloalkyl, wherein the C₃-C₆ cycloalkyl is optionally substituted with one or more —NH₂;
each R⁸ is independently hydrogen, cyclopropyl, or C₁₋₄ alkyl, wherein the C₁₋₄ alkyl is optionally substituted with one or more —F;
each R⁹ is independently C₁ fluoroalkyl or —CH₃;
each R¹⁰ is independently hydrogen or —CH₃; and
each R¹¹ is independently hydrogen, C₁₋₅ alkyl, or —CH₂-cyclopropyl; and
m is 1 or 2.

In some embodiments, provided is a compound of formula (III), or a stereoisomer, tautomer, pharmaceutically acceptable salt or solvate thereof, wherein X is O.

In some embodiments, provided is a compound of formula (III), or a stereoisomer, tautomer, pharmaceutically acceptable salt or solvate thereof, wherein X is S.

In some embodiments, provided is a compound of formula (III), or a stereoisomer, tautomer, pharmaceutically acceptable salt or solvate thereof, wherein R¹ is phenyl optionally substituted with one or more substituents independently selected from the group consisting of C₁-C₄ alkyl, —F, C₁ fluoroalkyl, C₁₋₄ hydroxyalkyl, —CON(R⁸)₂, and —C(O)R⁹; each R⁸ is independently hydrogen, cyclopropyl, or C₁₋₄ alkyl, wherein the C₁₋₄ alkyl is optionally substituted with one or more —F; and each R⁹ is independently C₁ fluoroalkyl.

In some embodiments, provided is a compound of formula (III), or a stereoisomer, tautomer, pharmaceutically acceptable salt or solvate thereof, wherein R¹ is phenyl optionally substituted with —COOCH₃, —C(O)N(H)CH₂CH₃, —C(O)N(H)CH₂CF₃, —OCH₃, and

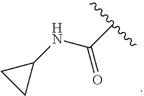 [SMILES: *C(=O)NC1CC1]

In some embodiments, provided is a compound of formula (III), or a stereoisomer, tautomer, pharmaceutically acceptable salt or solvate thereof, wherein R¹ is —C(O)R⁵, and R⁵ is cyclopropyl optionally substituted with one or more substituents independently selected from the group consisting of —F, —CH₃, C₁ fluoroalkyl, and phenyl.

In some embodiments, provided is a compound of formula (III), or a stereoisomer, tautomer, pharmaceutically acceptable salt or solvate thereof, wherein R¹ is —C(O)R⁵, and R⁵ is cyclopropyl.

In some embodiments, provided is a compound of formula (III), or a stereoisomer, tautomer, pharmaceutically acceptable salt or solvate thereof, wherein R¹ is —C(O)R⁵, and R⁵ is cyclopropyl substituted with 1-2 fluoro atoms.

In some embodiments, provided is a compound of formula (III), or a stereoisomer, tautomer, pharmaceutically acceptable salt or solvate thereof, wherein R¹ is —C(O)R⁵, and R⁵ is cyclopropyl substituted with —CH₃.

In some embodiments, provided is a compound of formula (III), or a stereoisomer, tautomer, pharmaceutically acceptable salt or solvate thereof, wherein R¹ is —C(O)R⁵, and R⁵ is cyclopropyl substituted with —CF₃.

In some embodiments, provided is a compound of formula (III), or a stereoisomer, tautomer, pharmaceutically acceptable salt or solvate thereof, wherein R¹ is —C(O)R⁵, and R⁵ is cyclopropyl substituted with phenyl.

In some embodiments, provided is a compound of formula (III), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, and —OCH₃, wherein the 5- to 6-membered heteroaryl is optionally fused to a 5- to 6-membered heteroaryl having 1-2 nitrogen heteroatoms; wherein each R⁸ is independently hydrogen, cyclopropyl, or C₁₋₄ alkyl, wherein the C₁₋₄ alkyl is optionally substituted with one or more —F, each R⁹ is independently C₁ fluoroalkyl or —CH₃, and each R¹⁰ is independently hydrogen or —CH₃.

In some embodiments, provided is a compound of formula (III), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, and cyclopropyl; wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃.

In some embodiments, provided is a compound of formula (III), or a stereoisomer, tautomer, pharmaceutically acceptable salt or solvate thereof, wherein R¹ is

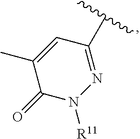

wherein R¹¹ is hydrogen, —CH₃, —CH₂CH₃, or —CH₂-cyclopropyl.

In some embodiments, provided is a compound of formula (III), or a stereoisomer, tautomer, pharmaceutically acceptable salt or solvate thereof, wherein R¹ is a 5- to 6-membered heteroaryl having 1-2 nitrogen heteroatoms optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, and —OCH₃, wherein the 5- to 6-membered heteroaryl is fused to a 5- to 6-membered heteroaryl having 1-2 nitrogen heteroatoms; wherein each R⁸ is independently hydrogen, cyclopropyl, or C₁₋₄ alkyl, wherein the C₁₋₄ alkyl is optionally substituted with one or more —F, each R⁹ is independently C₁ fluoroalkyl or —CH₃, and each R¹⁰ is independently hydrogen or —CH₃.

In some embodiments, provided is a compound of formula (III), or a stereoisomer, tautomer, pharmaceutically acceptable salt or solvate thereof, wherein R¹ is pyrrolo[2,3-b]pyridine or imidazo[1,2-b]pyridazine.

In some embodiments, provided is a compound of formula (III), or a stereoisomer, tautomer, pharmaceutically acceptable salt or solvate thereof, wherein R¹ is pyrazolyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, and cyclopropyl, wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃.

In some embodiments, provided is a compound of formula (III), or a stereoisomer, tautomer, pharmaceutically acceptable salt or solvate thereof, wherein R¹ is pyrazolyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, and cyclopropyl.

In some embodiments, provided is a compound of formula (III), or a stereoisomer, tautomer, pharmaceutically acceptable salt or solvate thereof, wherein R¹ is pyridinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, and cyclopropyl, wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃.

In some embodiments, provided is a compound of formula (III), or a stereoisomer, tautomer, pharmaceutically acceptable salt or solvate thereof, wherein R¹ is pyridinyl optionally substituted with one or more substituents independently selected from the group consisting of —C(O)N(CH₃)₂, —C(O)NHCH₃, —F, —CH₃, —CHF₂, —C(O)CF₃, —CH₂C(OH)(CH₃)₂, —CH(CH₃)₂, and cyclopropyl.

In some embodiments, provided is a compound of formula (III), or a stereoisomer, tautomer, pharmaceutically acceptable salt or solvate thereof, wherein R¹ is pyrazinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, and cyclopropyl, wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃.

In some embodiments, provided is a compound of formula (III), or a stereoisomer, tautomer, pharmaceutically acceptable salt or solvate thereof, wherein R¹ is pyrazinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, and cyclopropyl.

In some embodiments, provided is a compound of formula (III), or a stereoisomer, tautomer, pharmaceutically acceptable salt or solvate thereof, wherein R¹ is pyrimidinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, and cyclopropyl, wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃.

In some embodiments, provided is a compound of formula (III), or a stereoisomer, tautomer, pharmaceutically acceptable salt or solvate thereof, wherein R¹ is pyrimidinyl optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, —C(CH₃)₃, and cyclopropyl.

In some embodiments, provided is a compound of formula (III), or a stereoisomer, tautomer, pharmaceutically acceptable salt or solvate thereof, wherein R¹ is pyridazinyl optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, —CH₂OCH₃, cyclopropyl, and —N(R¹⁰)₂, wherein each R⁸ is independently hydrogen, cyclopropyl, or C₁₋₄ alkyl, wherein the C₁₋₄ alkyl is optionally substituted with one or more —F, and each R⁹ is independently C₁ fluoroalkyl or —CH₃, and each R¹⁰ is independently hydrogen or —CH₃.

In some embodiments, provided is a compound of formula (III), or a stereoisomer, tautomer, pharmaceutically acceptable salt or solvate thereof, wherein R¹ is pyridazinyl optionally substituted with one or more substituents independently selected from the group consisting of —C(O)NCH₃, —F, —CH₃, —CHF₂, —C(O)CF₃, —CH₂C(OH)(CH₃)₂, —CH₂OCH₃, —CH₂CH₃, —CH(CH₃)₂, —NHCH₃, and cyclopropyl.

In some embodiments, provided is a compound of formula (III), or a stereoisomer, tautomer, pharmaceutically acceptable salt or solvate thereof, wherein R¹ is phenyl optionally substituted with —COOR⁸, —CON(R⁸)₂, and —OCH₃,

In some embodiments, provided is a compound of formula (III), or a stereoisomer, tautomer, pharmaceutically acceptable salt or solvate thereof, wherein R¹ is phenyl optionally substituted with —COOCH₃, —C(O)N(H)CH₂CH₃, —C(O)N(H)CH₂CF₃, —OCH₃, and

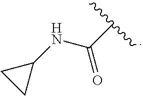 [SMILES: *C(=O)NC1CC1]

In some embodiments, provided is a compound of formula (III), or a stereoisomer, tautomer, pharmaceutically acceptable salt or solvate thereof, wherein R¹ is:

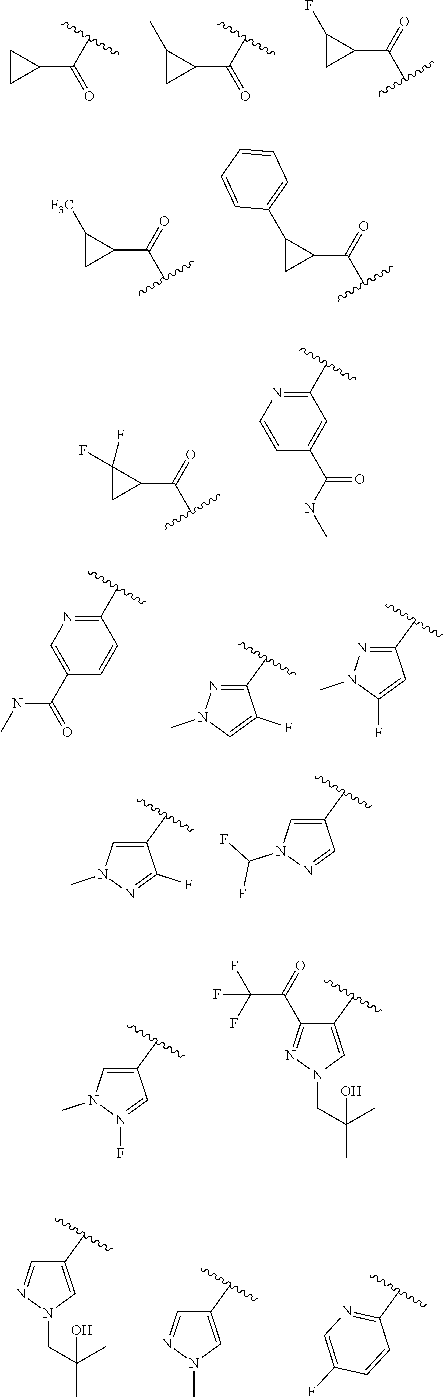

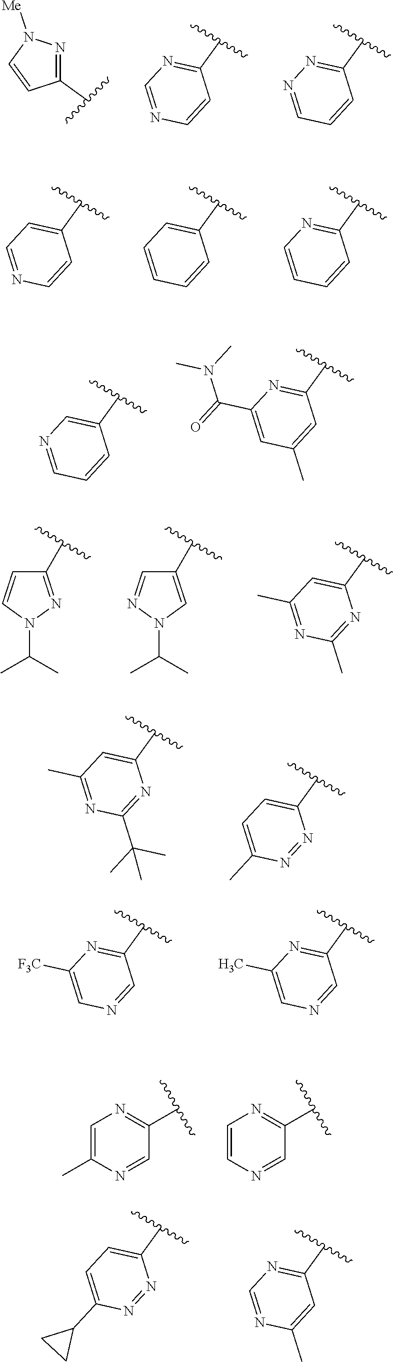

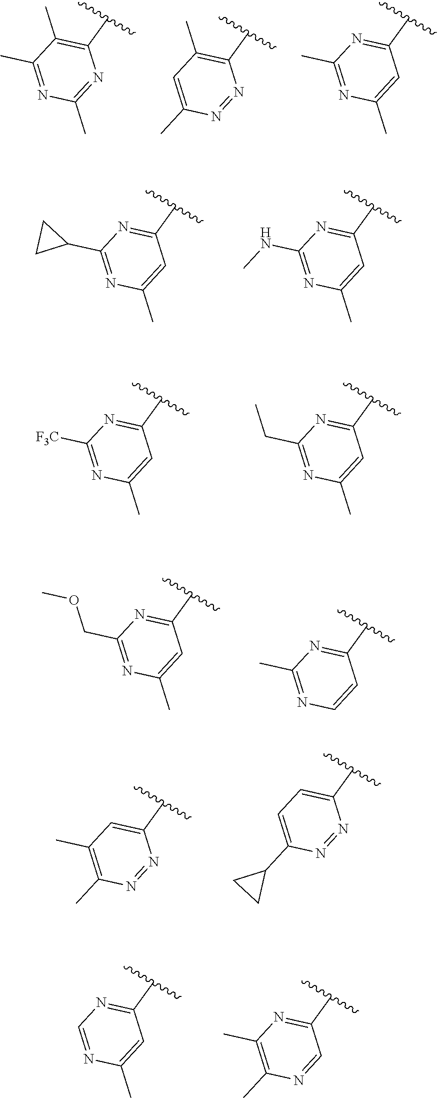

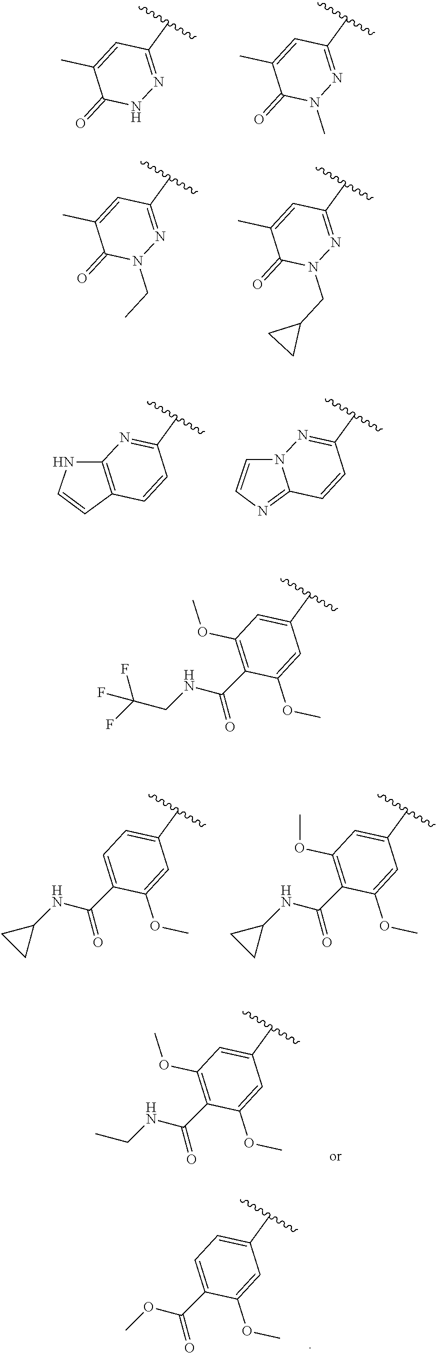

In some embodiments, provided is a compound of formula (III), or a stereoisomer, tautomer, pharmaceutically acceptable salt or solvate thereof, wherein R² is a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl; wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (III), or a stereoisomer, tautomer, pharmaceutically acceptable salt or solvate thereof, wherein R² is a 4- to 9-membered monocyclic or bicyclic heterocyclyl having 1 to 2 heteroatoms independently selected from oxygen and nitrogen, wherein the 4- to 9-membered monocyclic or bicyclic heterocyclyl is optionally substituted with one or more substituents independently selected from the group consisting of —CH₃, —OCH₃, —CH₂CH₃, —CH(CH₃)₂, —CHF₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

In some embodiments, provided is a compound of formula (III), or a stereoisomer, tautomer, pharmaceutically acceptable salt or solvate thereof, wherein R² is a 4- to 7-membered monocyclic heterocyclyl having 1 to 2 heteroatoms independently selected from oxygen and nitrogen, wherein the 4- to 7-membered monocyclic heterocyclyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl; wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (III), or a stereoisomer, tautomer, pharmaceutically acceptable salt or solvate thereof, wherein R² is a 4- to 7-membered monocyclic heterocyclyl having 1 to 2 heteroatoms independently selected from oxygen and nitrogen, wherein the 4- to 7-membered monocyclic heterocyclyl is optionally substituted with one or more substituents independently selected from the group consisting of —CH₃, —OCH₃, —CH₂CH₃, —CH(CH₃)₂, —CHF₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

In some embodiments, provided is a compound of formula (III), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, and oxetanyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl; wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (III), or a stereoisomer, tautomer, pharmaceutically acceptable salt or solvate thereof, wherein R² is a 4- to 7-membered monocyclic heterocyclyl selected from the group consisting of pyrrolidinyl, piperidinyl, oxazepanyl, morpholinyl, azetidinyl, and oxetanyl, wherein each of the pyrrolidinyl, piperidinyl, oxazepanyl, morpholinyl, azetidinyl, and oxetanyl is optionally substituted with one or more substituents independently selected from the group consisting of —CH₃, —OCH₃, —CH₂CH₃, —CH(CH₃)₂, —CHF₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

In some embodiments, provided is a compound of formula (III), or a stereoisomer, tautomer, pharmaceutically acceptable salt or solvate thereof, wherein R² is a 4- to 7-membered monocyclic heterocyclyl selected from the group consisting of:

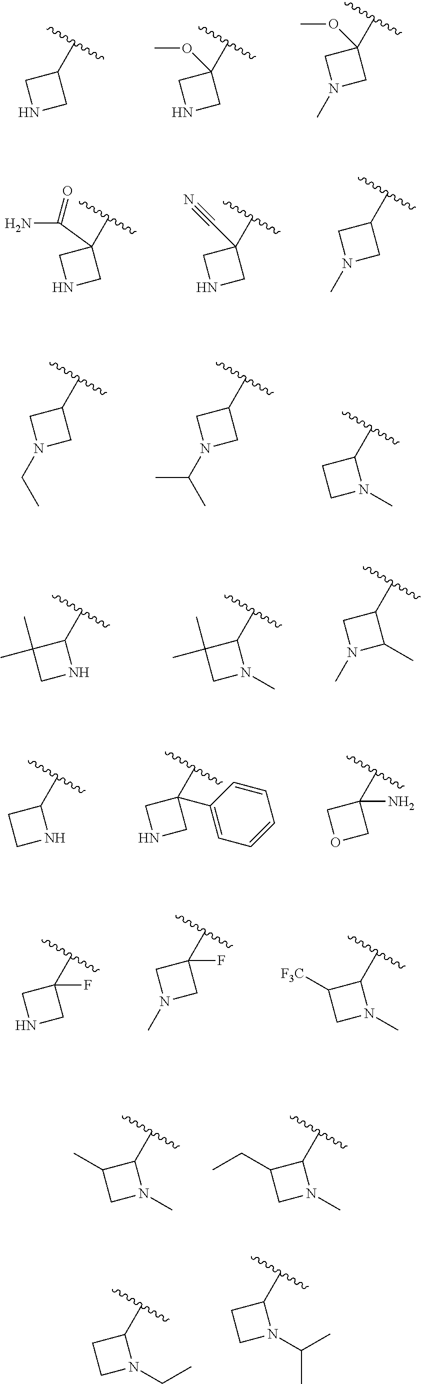

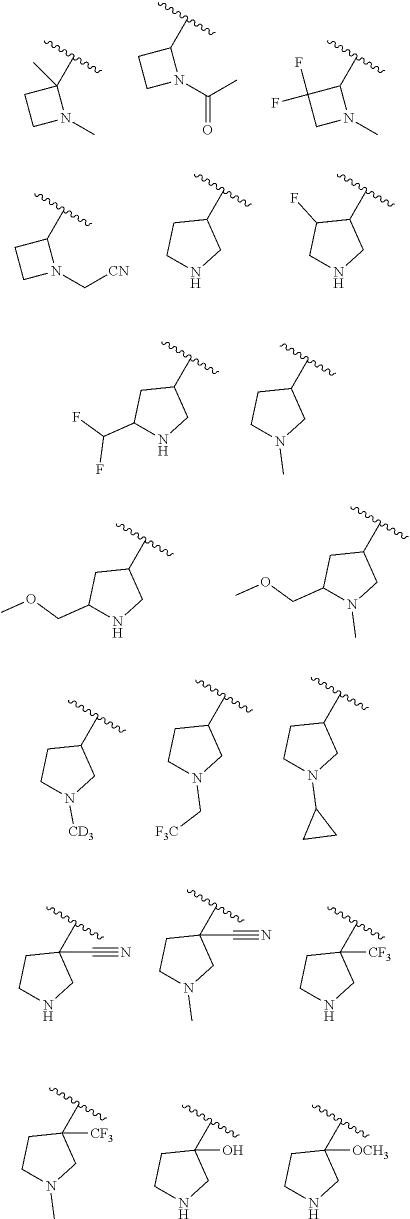

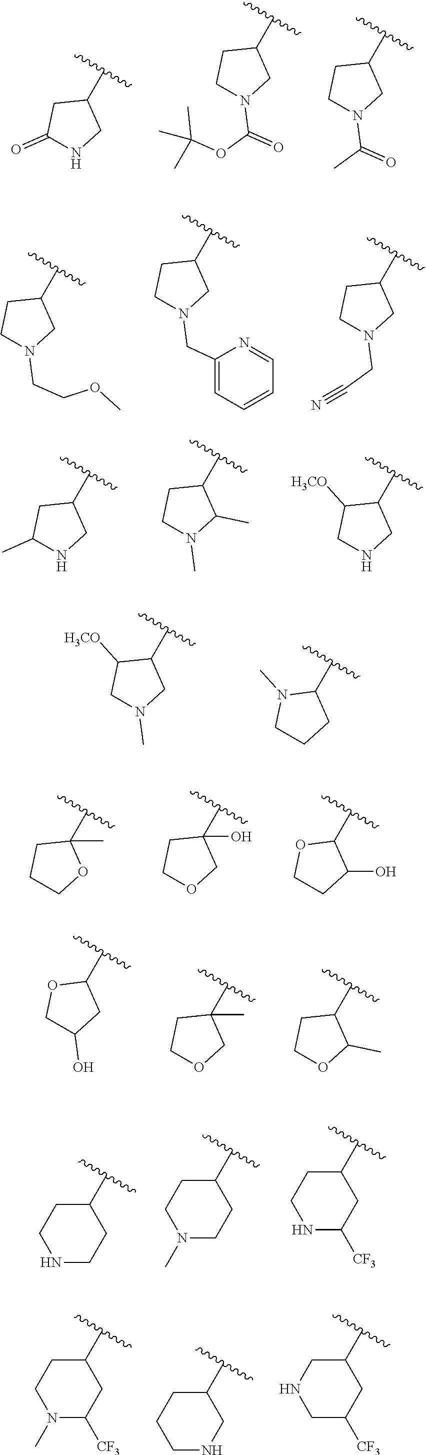

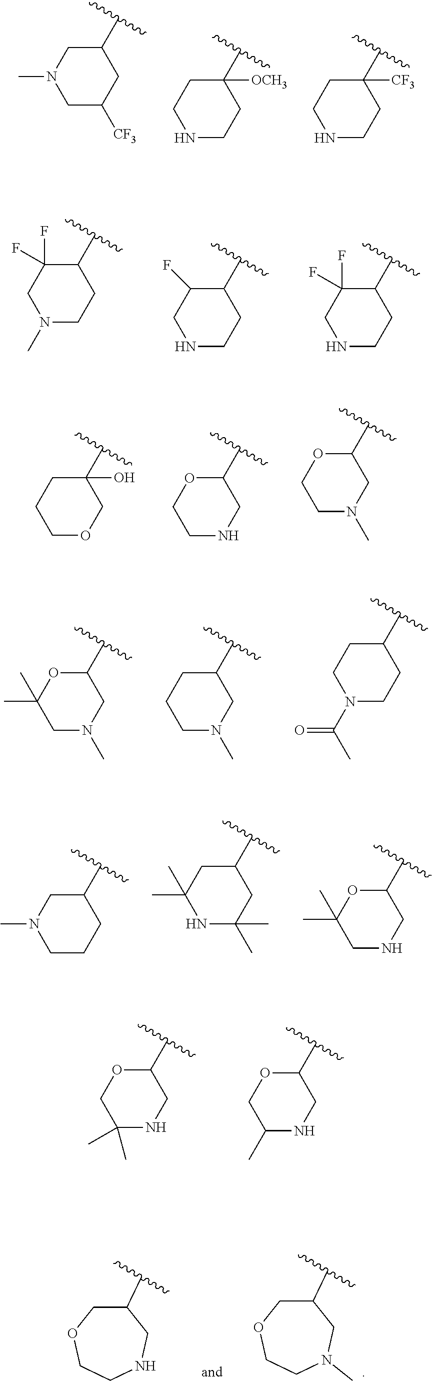

In some embodiments, provided is a compound of formula (III), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of C₁₋₃ alkyl, C₁₋₂ fluoroalkyl, —F, —OH, —OCH₃, oxo, cyclopropyl, —COOR⁸, —C(O)CH₃, —N(R¹⁰)₂, —(CH₂)ₘOCH₃, —CN, —CH₂CN, —CH₂(C₅H₄N), —CD₃, —CH₂—OCH₃, —CONH₂, and phenyl; wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (III), or a stereoisomer, tautomer, pharmaceutically acceptable salt or solvate thereof, wherein R² is a 6- to 9-membered bicyclic heterocyclyl having 1 to 2 heteroatoms independently selected from oxygen and nitrogen, wherein the 6- to 9-membered bicyclic heterocyclyl is optionally substituted with one or more substituents independently selected from the group consisting of —CH₃, —OCH₃, —CH₂CH₃, —CH(CH₃)₂, —CHF₂, —CH₂CF₃, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

In some embodiments, provided is a compound of formula (III), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl is optionally substituted with one or more substituents independently selected from the group consisting of C₁₋₃ alkyl, C₁₋₂ fluoroalkyl, —F, —OH, —OCH₃, oxo, —CN, cyclopropyl, —COOR⁸, —C(O)CH₃, —N(R¹⁰)₂, —(CH₂)ₘOCH₃, —CH₂CN, —CH₂(C₅H₄N), and phenyl, wherein each wherein each R⁸ is independently hydrogen, cyclopropyl, or C₁₋₄ alkyl, wherein the C₁₋₄ alkyl is optionally substituted with one or more —F, each R¹⁰ is independently hydrogen or —CH₃, and m is 1 or 2.

In some embodiments, provided is a compound of formula (III), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl is optionally substituted with one or more substituents independently selected from the group consisting of —CH₃, —OCH₃, —CH₂CH₃, —CH(CH₃)₂, —CH₂F, —CH₂CF₃, —CF₃, —F, —OH, —NH₂, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

In some embodiments, provided is a compound of formula (III), or a stereoisomer, tautomer, pharmaceutically acceptable salt or solvate thereof, wherein R² is a 6- to 9-membered bicyclic heterocyclyl selected from the group consisting of 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 2-aza-bicyclo[2.2.1]heptane, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, 5-azaspiro[2.3]hexanyl, 2-oxaspiro[4.4]nonane, 6-oxaspiro[3.4]octane, 1-oxaspiro[4.4]nonane, 5-oxa-2-azabicyclo[2.2.1]heptanyl, 9-azaspiro[3.5]nonanyl, and 3-(1,2,3,3a,5,6,7,7a-octahydropyrano[3,2-b]pyrrolyl is optionally substituted with one or more substituents independently selected from the group consisting of —CH₃, —CH₂CH₃, —COOC(CH₃)₃, and oxo.

In some embodiments, provided is a compound of formula (III), or a stereoisomer, tautomer, pharmaceutically acceptable salt or solvate thereof, wherein R² is a 6- to 9-membered bicyclic heterocyclyl selected from the group consisting of:

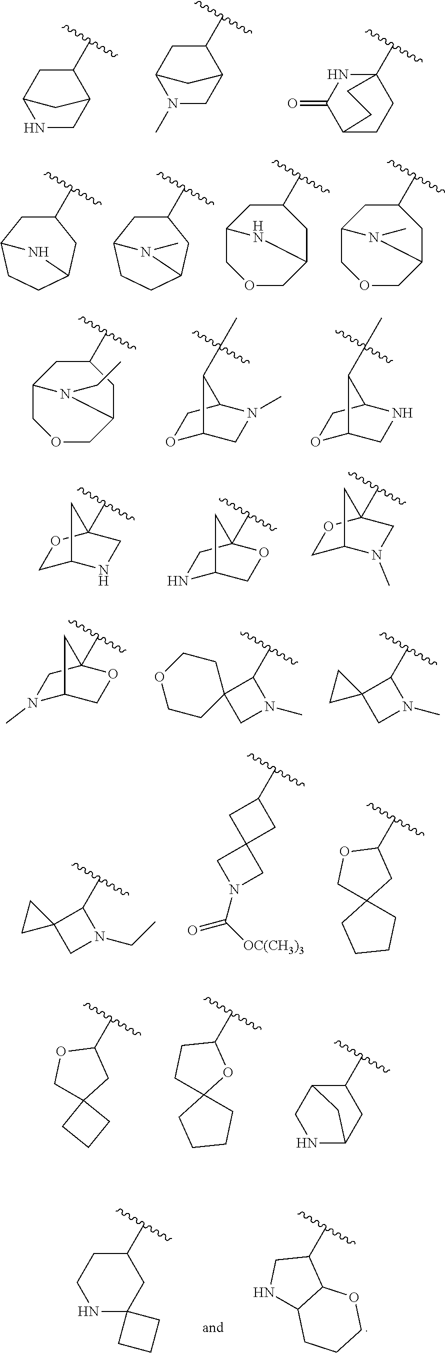

In some embodiments, provided is a compound of formula (III), or a stereoisomer, tautomer, pharmaceutically acceptable salt or solvate thereof, wherein R² is a C₁₋₅ alkyl optionally substituted with one or more substituents independently selected from the group consisting of —N(CH₃)₂, —OH, —CF₃, —NH₂, —COOC(CH₃)₃, cyclopropyl, —CH₃, C₃-C₆ cycloalkyl, phenyl, flurophenyl, pyridyl, and —CN.

In some embodiments, provided is a compound of formula (III), or a stereoisomer, tautomer, pharmaceutically acceptable salt or solvate thereof, wherein R² is selected from the group consisting of —CH₂CH₂N(CH₃)₂, —C(OH)(CH₃)CF₃, —C(CH₃)₂OH, —CH(OH)CH₃, —CH(OH)CF₃, —C(OH)(CF₃)₂, —CH(OH)CH₃, —CH(NH₂)C(CH₃)₃, —CH₂C(O)OC(CH₃)₃, and —C(CH₃)(NH₂)CF₃, —CH₂CH(OH)(C₆H₅), —CH₂C(OH)(CH₃)CF₃, —CH₂C(OH)(CH₃)(C₃H₅), —CH₂CH₂(C₅H₄N). —CH₂CH(OH)(C₆H₁₁), —CH₂C(OH)(CH₃)₂, —CH₂CH(NH₂)(C₆H₅), and —CH₂C(OH)(CF₃)₂.

In some embodiments, provided is a compound of formula (III), or a stereoisomer, tautomer, pharmaceutically acceptable salt or solvate thereof, wherein R² is pyridinyl or pyrazolyl optionally substituted with one or more substituents independently selected from the group consisting of —CF₃ and —CH₃.

In some embodiments, provided is a compound of formula (III), or a stereoisomer, tautomer, pharmaceutically acceptable salt or solvate thereof, wherein R² is pyridinyl substituted with —CF₃.

In some embodiments, provided is a compound of formula (III), or a stereoisomer, tautomer, pharmaceutically acceptable salt or solvate thereof, wherein R² is pyridinyl.

In some embodiments, provided is a compound of formula (III), or a stereoisomer, tautomer, pharmaceutically acceptable salt or solvate thereof, wherein R² is pyrazolyl substituted with —CH₃.

In some embodiments, provided is a compound of formula (III), or a stereoisomer, tautomer, pharmaceutically acceptable salt or solvate thereof, wherein R² is pyrazolyl.

In some embodiments, provided is a compound of formula (III), or a stereoisomer, tautomer, pharmaceutically acceptable salt or solvate thereof, wherein R² is:

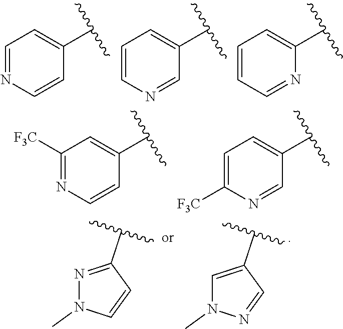

In some embodiments, provided is a compound of formula (II), or a stereoisomer, tautomer, pharmaceutically acceptable salt or solvate thereof, wherein R² is a C₃-C₆ cycloalkyl, wherein the C₃-C₆ cycloalkyl is optionally substituted with one or more —NH₂.

In some embodiments, provided is a compound of formula (II), or a stereoisomer, tautomer, pharmaceutically acceptable salt or solvate thereof, wherein R² is a cyclobutyl, wherein the cyclobutyl is optionally substituted with one or more —NH₂.

In some embodiments, provided is a compound of formula (III), or a stereoisomer, tautomer, pharmaceutically acceptable salt or solvate thereof, wherein Z is —O—(CH₂)ₙ—, provided that O is directly bonded to the isoxazolyl ring or isothiazolyl ring. In one embodiment, n is 1. In another embodiment, n is 0. Thus, in some embodiments Z is —O— and in other embodiments Z is —OCH₂—.

In some embodiments, provided is a compound of formula (III), or a stereoisomer, tautomer, pharmaceutically acceptable salt or solvate thereof, wherein each R⁸ is independently hydrogen, —CH₃, —C(CH₃)₃, cyclopropyl, —CH₂CH₃, or —CH₂CF₃.

In some embodiments, provided is a compound of formula (III), or a stereoisomer, tautomer, pharmaceutically acceptable salt or solvate thereof, wherein R⁹ is —CF₃.

In some embodiments, provided is a compound of formula (III), or a stereoisomer, tautomer, pharmaceutically acceptable salt or solvate thereof, wherein R⁹ is —CH₃.

In some embodiments, provided is a compound of formula (III), or a stereoisomer, tautomer, pharmaceutically acceptable salt or solvate thereof, wherein R¹¹ is hydrogen, —CH₃, —CH₂CH₃, and —CH₂(C₃H₅).

All possible combinations of the above-indicated embodiments of compounds of formula (III) and their tautomers, stereoisomers, pharmaceutically acceptable salts and solvates are considered to be embraced within the scope of this application.

Exemplary compounds of formula (II) and compounds of formula (III) include, but are not limited to:
N-(5-(4-(((3S,5S)-1,5-dimethylpyrrolidin-3-yl)oxy)-1-methyl-1H-pyrazol-5-yl)pyrazolo[1,5-a]pyridin-2-yl)cyclopropanecarboxamide;
N-(2,6-dimethylpyrimidin-4-yl)-5-[2-methyl-4-[[(2R)-1-methylazetidin-2-yl]methoxy]pyrazol-3-yl]pyrazolo[1,5-a]pyridin-2-amine;
N-[5-[2-methyl-4-[[(2S)-1,3,3-trimethylazetidin-2-yl]methoxy]pyrazol-3-yl]pyrazolo[1,5-a]pyridin-2-yl]cyclopropanecarboxamide;
N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide;
N-(5-(4-(((3*R,4*S)-4-methoxypyrrolidin-3-yl)oxy)-1-methyl-1H-pyrazol-5-yl)pyrazolo[1,5-a]pyridin-2-yl)cyclopropanecarboxamide;
N-[5-[2-methyl-4-[[(2R)-morpholin-2-yl]methoxy]pyrazol-3-yl]pyrazolo[1,5-a]pyridin-2-yl]cyclopropanecarboxamide;
5-[2-methyl-4-[(3S)-1-methylpyrrolidin-3-yl]oxy-pyrazol-3-yl]-N-[6-(trifluoromethyl)pyrazin-2-yl]pyrazolo[1,5-a]pyridin-2-amine;
N-[5-[2-methyl-4-[(1-methylazetidin-3-yl)methoxy]pyrazol-3-yl]pyrazolo[1,5-a]pyridin-2-yl]cyclopropanecarboxamide;
N-[5-[2-methyl-4-[[(2S)-1-methylazetidin-2-yl]methoxy]pyrazol-3-yl]pyrazolo[1,5-a]pyridin-2-yl]cyclopropanecarboxamide;
N-[5-[2-methyl-4-[[(2R,3R)-1-methyl-3-(trifluoromethyl)azetidin-2-yl]methoxy]pyrazol-3-yl]pyrazolo[1,5-a]pyridin-2-yl]cyclopropanecarboxamide;
N-[5-[4-[[(2S,3R)-3-ethyl-1-methyl-azetidin-2-yl]methoxy]-2-methyl-pyrazol-3-yl]pyrazolo[1,5-a]pyridin-2-yl]cyclopropanecarboxamide;
N-[5-[4-[[(2S,3S)-1,3-dimethylazetidin-2-yl]methoxy]-2-methyl-pyrazol-3-yl]pyrazolo[1,5-a]pyridin-2-yl]cyclopropanecarboxamide;
(rac)-N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide;
(1R,2R)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide;
(1S,2S)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide;
(*R)—N-(5-(4-((5-Ethyl-5-azaspiro[2.3]hexan-4-yl)methoxy)-1-methyl-1H-pyrazol-5-yl)pyrazolo[1,5-a]pyridin-2-yl)cyclopropanecarboxamide;
N-(5,6-Dimethylpyridazin-3-yl)-5-[2-methyl-4-[[(2R)-1-methylazetidin-2-yl]methoxy]pyrazol-3-yl]pyrazolo[1,5-a]pyridin-2-amine;
(R)—N-(5,6-Dimethylpyridazin-3-yl)-5-(4-((1-ethylazetidin-2-yl)methoxy)-1-methyl-1H-pyrazol-5-yl)pyrazolo[1,5-a]pyridin-2-amine;
5-[4-[[(2R)-1-Ethylazetidin-2-yl]methoxy]-2-methyl-pyrazol-3-yl]-N-(6-methylpyrimidin-4-yl)pyrazolo[1,5-a]pyridin-2-amine;
(R)-5-(4-((1-Ethylazetidin-2-yl)methoxy)-1-methyl-1H-pyrazol-5-yl)-N-(6-methylpyrazin-2-yl)pyrazolo[1,5-a]pyridin-2-amine;
(R)-5-(4-((1-Ethylazetidin-2-yl)methoxy)-1-methyl-1H-pyrazol-5-yl)-N-(5-methylpyrazin-2-yl)pyrazolo[1,5-a]pyridin-2-amine;
(R)—N-(5,6-Dimethylpyrazin-2-yl)-5-(4-((1-ethylazetidin-2-yl)methoxy)-1-methyl-1H-pyrazol-5-yl)pyrazolo[1,5-a]pyridin-2-amine;
N-(5,6-Dimethylpyrazin-2-yl)-5-[2-methyl-4-[[(2R)-1-methylazetidin-2-yl]methoxy]pyrazol-3-yl]pyrazolo[1,5-a]pyridin-2-amine;
N-[5-[4-[[(2R,3R)-1,3-Dimethylazetidin-2-yl]methoxy]-2-methyl-pyrazol-3-yl]pyrazolo[1,5-a]pyridin-2-yl]cyclopropanecarboxamide;
(1S,2S)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide;
1-methyl-N-[5-[2-methyl-4-[[(2R)-1-methylazetidin-2-yl]methoxy]pyrazol-3-yl]pyrazolo[1,5-a]pyridin-2-yl]pyrazole-4-carboxamide;
N-[5-[4-[(2S)-2-hydroxy-2-phenyl-ethoxy]-2-methyl-pyrazol-3-yl]pyrazolo[1,5-a]pyridin-2-yl]cyclopropanecarboxamide;
N-[5-[5-methyl-3-[[(2S)-morpholin-2-yl]methoxy]isoxazol-4-yl]pyrazolo[1,5-a]pyridin-2-yl]cyclopropanecarboxamide;
N-[5-[3-[(3-methoxy-1-methyl-azetidin-3-yl)methoxy]-5-methyl-isoxazol-4-yl]pyrazolo[1,5-a]pyridin-2-yl]cyclopropanecarboxamide;
N-(2,6-dimethylpyrimidin-4-yl)-5-[3-[(3-methoxyazetidin-3-yl)methoxy]-5-methyl-isoxazol-4-yl]pyrazolo[1,5-a]pyridin-2-amine;
N-[5-[3-[(2S)-2-amino-2-phenyl-ethoxy]-5-methyl-isoxazol-4-yl]pyrazolo[1,5-a]pyridin-2-yl]cyclopropanecarboxamide;
5-[2-(difluoromethyl)-4-[(3R,4S)-4-fluoropyrrolidin-3-yl]oxy-pyrazol-3-yl]-N-(2,6-dimethylpyrimidin-4-yl)pyrazolo[1,5-a]pyridin-2-amine;
6-[[5-[2-(difluoromethyl)-4-[[(2R)-1-methylazetidin-2-yl]methoxy]pyrazol-3-yl]pyrazolo[1,5-a]pyridin-2-yl]amino]-2,4-dimethyl-pyridazin-3-one;
(*S)-3-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one;
*R-4-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one;
N-(2-cyano-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-isopropyl-2,6-dimethoxy-4-[[5-[2-methyl-4-[[(2R)-1-methylazetidin-2-yl]methoxy]pyrazol-3-yl]pyrazolo[1,5-a]pyridin-2-yl]amino]benzamide;
N-[5-[2-methyl-4-[[(2R)-1-methylazetidin-2-yl]methoxy]pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(6-methylpyridazin-3-yl)pyrazolo[1,5-a]pyridin-2-amine;
N-(5-(4-(((3*S,4*R)-4-methoxypyrrolidin-3-yl)oxy)-1-methyl-1H-pyrazol-5-yl)pyrazolo[1,5-a]pyridin-2-yl)cyclopropanecarboxamide;
trans-N-(5-(4-(((2*R,3*S)-1,2-dimethylazetidin-3-yl)methoxy)-1-methyl-1H-pyrazol-5-yl)pyrazolo[1,5-a]pyridin-2-yl)cyclopropanecarboxamide;
5-[4-[[(2R)-azetidin-2-yl]methoxy]-2-methyl-pyrazol-3-yl]-N-(2,6-dimethylpyrimidin-4-yl)pyrazolo[1,5-a]pyridin-2-amine;
exo-N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide;
N-[5-[2-methyl-4-[(3-phenylazetidin-3-yl)methoxy]pyrazol-3-yl]pyrazolo[1,5-a]pyridin-2-yl]cyclopropanecarboxamide;
5-[4-[[(2R)-azetidin-2-yl]methoxy]-2-methyl-pyrazol-3-yl]-N-(6-methylpyrazin-2-yl)pyrazolo[1,5-a]pyridin-2-amine;
5-[2-methyl-4-[[(2R)-1-methylazetidin-2-yl]methoxy]pyrazol-3-yl]-N-(6-methylpyrazin-2-yl)pyrazolo[1,5-a]pyridin-2-amine;
N-[5-[4-[[(2S)-1-ethylazetidin-2-yl]methoxy]-2-methyl-pyrazol-3-yl]pyrazolo[1,5-a]pyridin-2-yl]cyclopropanecarboxamide;
N-[5-[4-[[(2S)-1-isopropylazetidin-2-yl]methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(5-methylpyrazin-2-yl)pyrazolo[1,5-a]pyridin-2-amine;
N-(6-cyclopropylpyridazin-3-yl)-5-[2-methyl-4-[[(2R)-1-methylazetidin-2-yl]methoxy]pyrazol-3-yl]pyrazolo[1,5-a]pyridin-2-amine;
(rac)-N-[5-[2-methyl-4-[(5-methyl-5-azaspiro[2.3]hexan-6-yl)methoxy]pyrazol-3-yl]pyrazolo[1,5-a]pyridin-2-yl]cyclopropanecarboxamide;
N-[5-[4-[[(2R)-1-ethylazetidin-2-yl]methoxy]-2-methyl-pyrazol-3-yl]pyrazolo[1,5-a]pyridin-2-yl]cyclopropanecarboxamide;
N-[5-[4-[[(2R)-1-isopropylazetidin-2-yl]methoxy]-2-methyl-pyrazol-3-yl]pyrazolo[1,5-a]pyridin-2-yl]cyclopropanecarboxamide;
(rac)-N-[5-[4-[(1,2-dimethylazetidin-2-yl)methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(6-methylpyrimidin-4-yl)pyrazolo[1,5-a]pyridin-2-amine;
N-[5-[4-[[(2R)-1-acetylazetidin-2-yl]methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(2,5,6-trimethylpyrimidin-4-yl)pyrazolo[1,5-a]pyridin-2-amine;
(*R)—N-(5-(1-methyl-4-((2-methyl-7-oxa-2-azaspiro[3.5]nonan-1-yl)methoxy)-1H-pyrazol-5-yl)pyrazolo[1,5-a]pyridin-2-yl)cyclopropanecarboxamide;
(*S)—N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide;
rac-N-[5-[4-[(3,3-difluoro-1-methyl-azetidin-2-yl)methoxy]-2-methyl-pyrazol-3-yl]pyrazolo[1,5-a]pyridin-2-yl]cyclopropanecarboxamide;
N-(4,6-dimethylpyridazin-3-yl)-5-[2-methyl-4-[[(2R)-1-methylazetidin-2-yl]methoxy]pyrazol-3-yl]pyrazolo[1,5-a]pyridin-2-amine;
N-(2,6-dimethylpyrimidin-4-yl)-5-[4-[[(2R)-1-ethylazetidin-2-yl]methoxy]-2-methyl-pyrazol-3-yl]pyrazolo[1,5-a]pyridin-2-amine;
5-[2-methyl-4-[[(2R)-1-methylazetidin-2-yl]methoxy]pyrazol-3-yl]-N-[6-methyl-2-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-(2-methylpyrimidin-4-yl)pyrazolo[1,5-a]pyridin-2-amine;
5-[4-[[(2R)-1-ethylazetidin-2-yl]methoxy]-2-methyl-pyrazol-3-yl]-N-(2-methylpyrimidin-4-yl)pyrazolo[1,5-a]pyridin-2-amine;
(1R,2R)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide;
(*S)—N-[5-[4-[(5-ethyl-5-azaspiro[2.3]hexan-6-yl)methoxy]-2-methyl-pyrazol-3-yl]pyrazolo[1,5-a]pyridin-2-yl]cyclopropanecarboxamide;
5-[4-[[(2R)-1-ethylazetidin-2-yl]methoxy]-2-methyl-pyrazol-3-yl]-N-(6-methylpyridazin-3-yl)pyrazolo[1,5-a]pyridin-2-amine;
(1S,2S)-2-fluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide;
trans-N-[5-[2-methyl-4-[((2R)-1-methylazetidin-2-yl)methoxy]pyrazol-3-yl]pyrazolo[1,5-a]pyridin-2-yl]-2-(trifluoromethyl)cyclopropanecarboxamide;
trans-N-[5-[2-methyl-4-[((2R)-1-methylazetidin-2-yl)methoxy]pyrazol-3-yl]pyrazolo[1,5-a]pyridin-2-yl]-2-phenyl-cyclopropanecarboxamide;
2,2-difluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide;
5-[4-[[(2R)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
N-[5-[5-methyl-3-[(3R)-pyrrolidin-3-yl]oxy-isoxazol-4-yl]pyrazolo[1,5-a]pyridin-2-yl]cyclopropanecarboxamide;
N-[5-[5-methyl-3-[(3S)-pyrrolidin-3-yl]oxy-isoxazol-4-yl]pyrazolo[1,5-a]pyridin-2-yl]cyclopropanecarboxamide;
N-[5-[3-[(3-methoxyazetidin-3-yl)methoxy]-5-methyl-isoxazol-4-yl]pyrazolo[1,5-a]pyridin-2-yl]cyclopropanecarboxamide;
N-[5-[3-[(3-cyanoazetidin-3-yl)methoxy]-5-methyl-isoxazol-4-yl]pyrazolo[1,5-a]pyridin-2-yl]cyclopropanecarboxamide;
N-[5-[5-methyl-3-[[(2R)-morpholin-2-yl]methoxy]isoxazol-4-yl]pyrazolo[1,5-a]pyridin-2-yl]cyclopropanecarboxamide;
N-[5-[5-methyl-3-[[(2R)-4-methylmorpholin-2-yl]methoxy]isoxazol-4-yl]pyrazolo[1,5-a]pyridin-2-yl]cyclopropanecarboxamide;
N-[5-[3-[(3S,4R)-4-fluoropyrrolidin-3-yl]oxy-5-methyl-isoxazol-4-yl]pyrazolo[1,5-a]pyridin-2-yl]cyclopropanecarboxamide;
N-[5-[5-methyl-3-[[(1S,4S)-5-oxa-2-azabicyclo[2.2.1]heptan-4-yl]methoxy]isoxazol-4-yl]pyrazolo[1,5-a]pyridin-2-yl]cyclopropanecarboxamide;
N-[5-[3-[(3S,4S)-4-fluoropyrrolidin-3-yl]oxy-5-methyl-isoxazol-4-yl]pyrazolo[1,5-a]pyridin-2-yl]cyclopropanecarboxamide;
N-[5-[3-[(3-fluoroazetidin-3-yl)methoxy]-5-methyl-isoxazol-4-yl]pyrazolo[1,5-a]pyridin-2-yl]cyclopropanecarboxamide;
N-[5-[3-[(3-fluoro-1-methyl-azetidin-3-yl)methoxy]-5-methyl-isoxazol-4-yl]pyrazolo[1,5-a]pyridin-2-yl]cyclopropanecarboxamide;
N-[5-[5-methyl-3-[(3S,5R)-5-methylpyrrolidin-3-yl]oxy-isoxazol-4-yl]pyrazolo[1,5-a]pyridin-2-yl]cyclopropanecarboxamide;
N-[5-[3-[[(2R)-6,6-dimethylmorpholin-2-yl]methoxy]-5-methyl-isoxazol-4-yl]pyrazolo[1,5-a]pyridin-2-yl]cyclopropanecarboxamide;
N-[5-[5-methyl-3-[[(2R)-1-methylazetidin-2-yl]methoxy]isoxazol-4-yl]pyrazolo[1,5-a]pyridin-2-yl]cyclopropanecarboxamide;
N-[5-[3-[(3R,4S)-4-methoxypyrrolidin-3-yl]oxy-5-methyl-isoxazol-4-yl]pyrazolo[1,5-a]pyridin-2-yl]cyclopropanecarboxamide;
N-[5-[3-[[(2S)-3,3-dimethylazetidin-2-yl]methoxy]-5-methyl-isoxazol-4-yl]pyrazolo[1,5-a]pyridin-2-yl]cyclopropanecarboxamide;
N-(2,6-dimethylpyrimidin-4-yl)-5-[5-methyl-3-[(3 S,5R)-5-methylpyrrolidin-3-yl]oxy-isoxazol-4-yl]pyrazolo[1,5-a]pyridin-2-amine;
N-(2,6-dimethylpyrimidin-4-yl)-5-[5-methyl-3-[[(2R)-1-methylazetidin-2-yl]methoxy]isoxazol-4-yl]pyrazolo[1,5-a]pyridin-2-amine;
N-(2,6-dimethylpyrimidin-4-yl)-5-[3-[(3R,4S)-4-fluoropyrrolidin-3-yl]oxy-5-methyl-isoxazol-4-yl]pyrazolo[1,5-a]pyridin-2-amine;
N-(5,6-dimethylpyridazin-3-yl)-5-[3-[(3R,4S)-4-fluoropyrrolidin-3-yl]oxy-5-methyl-isoxazol-4-yl]pyrazolo[1,5-a]pyridin-2-amine;
N-(2,6-dimethylpyrimidin-4-yl)-5-[3-[(3R,4S)-4-methoxypyrrolidin-3-yl]oxy-5-methyl-isoxazol-4-yl]pyrazolo[1,5-a]pyridin-2-amine;
N-(5,6-dimethylpyridazin-3-yl)-5-[3-[(3R,4S)-4-methoxypyrrolidin-3-yl]oxy-5-methyl-isoxazol-4-yl]pyrazolo[1,5-a]pyridin-2-amine;
5-[2-(difluoromethyl)-4-[(3R,4S)-4-methoxypyrrolidin-3-yl]oxy-pyrazol-3-yl]-N-(2,6-dimethylpyrimidin-4-yl)pyrazolo[1,5-a]pyridin-2-amine;
5-[2-(difluoromethyl)-4-[[(2R)-1-methylazetidin-2-yl]methoxy]pyrazol-3-yl]-N-(6-methoxy-5-methyl-pyridazin-3-yl)pyrazolo[1,5-a]pyridin-2-amine;
5-[2-(difluoromethyl)-4-[[(2R)-1-methylazetidin-2-yl]methoxy]pyrazol-3-yl]-N-(6-methylpyridazin-3-yl)pyrazolo[1,5-a]pyridin-2-amine;
(2R,3S)-2-[[1-(difluoromethyl)-5-[2-[(2,6-dimethylpyrimidin-4-yl)amino]pyrazolo[1,5-a]pyridin-5-yl]pyrazol-4-yl]oxymethyl]tetrahydrofuran-3-ol;
(RS)-5-[2-(difluoromethyl)-4-[(3-methoxypyrrolidin-3-yl)methoxy]pyrazol-3-yl]-N-(2,6-dimethylpyrimidin-4-yl)pyrazolo[1,5-a]pyridin-2-amine;
(RS)-5-[2-(difluoromethyl)-4-[(3-fluoropyrrolidin-3-yl)methoxy]pyrazol-3-yl]-N-(2,6-dimethylpyrimidin-4-yl)pyrazolo[1,5-a]pyridin-2-amine;
N-[2-(difluoromethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine;
N-[2-(difluoromethyl)-6-methyl-pyrimidin-4-yl]-5-[4-[[(2R)-1-ethylazetidin-2-yl]methoxy]-2-methyl-pyrazol-3-yl]pyrazolo[1,5-a]pyridin-2-amine;
5-[2-(difluoromethyl)-4-[[(2R)-1-ethylazetidin-2-yl]methoxy]pyrazol-3-yl]-N-(1-methylpyrazol-4-yl)pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]cyclopropanecarboxamide;
N-(2-hydroxy-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(2-methoxyethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N,N,2-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide;
N,4-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide;
N,N,4-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide;
N-(2-hydroxy-2-methyl-propyl)-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide;
N-isopropyl-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide;
N-(2-hydroxy-2-methyl-propyl)-4-methyl-6-[[5-[2-methyl-4-[[(2R)-1-methylazetidin-2-yl]methoxy]pyrazol-3-yl]pyrazolo[1,5-a]pyridin-2-yl]amino]pyridine-2-carboxamide;
N-[5-[2-methyl-4-[[(2R)-1-methylazetidin-2-yl]methoxy]pyrazol-3-yl]pyrazolo[1,5-a]pyridin-2-yl]-7,8-dihydro-5H-pyrano[4,3-b]pyridin-2-amine;
N-(2-methoxyethyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
5-[2-methyl-4-[[(2R)-1-methylazetidin-2-yl]methoxy]pyrazol-3-yl]-N-[6-methyl-2-(3-pyridyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine;
5-[2-methyl-4-[[(2R)-1-methylazetidin-2-yl]methoxy]pyrazol-3-yl]-N-[2-(trifluoromethyl)pyrimidin-4-yl]pyrazolo[1,5-a]pyridin-2-amine;
N-(2-methoxypyrimidin-4-yl)-5-[2-methyl-4-[[(2R)-1-methylazetidin-2-yl]methoxy]pyrazol-3-yl]pyrazolo[1,5-a]pyridin-2-amine;
N,2-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide;
N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine;
5-[2-methyl-4-[[(2R)-1-methylazetidin-2-yl]methoxy]pyrazol-3-yl]-N-[2-methyl-6-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-[6-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-(1-methyltriazol-4-yl)pyrazolo[1,5-a]pyridin-2-amine;
N-[5-[2-methyl-4-[[(2R)-1-methylazetidin-2-yl]methoxy]pyrazol-3-yl]pyrazolo[1,5-a]pyridin-2-yl]-1,6-naphthyridin-2-amine;
N-methyl-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(6-methoxypyrimidin-4-yl)-5-[2-methyl-4-[[(2R)-1-methylazetidin-2-yl]methoxy]pyrazol-3-yl]pyrazolo[1,5-a]pyridin-2-amine;
N2-cyclopropyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine;
N2-cyclopentyl-6-methyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine;
N-(2,6-dimethylpyrimidin-4-yl)-5-[2-ethyl-4-[[(2R)-1-methylazetidin-2-yl]methoxy]pyrazol-3-yl]pyrazolo[1,5-a]pyridin-2-amine;
N-(2,6-dimethylpyrimidin-4-yl)-5-[4-[[(2R)-1-methylazetidin-2-yl]methoxy]-2-(trideuteriomethyl)pyrazol-3-yl]pyrazolo[1,5-a]pyridin-2-amine;
N-(2-hydroxy-2-methyl-propyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
N-methyl-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
N-(2-cyclopropyl-6-methyl-pyrimidin-4-yl)-5-[2-methyl-4-[[(2S)-1-methylazetidin-2-yl]methoxy]pyrazol-3-yl]pyrazolo[1,5-a]pyridin-2-amine;
N-(2-cyanoethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(4-methyl-6,7-dihydro-5H-cyclopenta[b]pyridin-2-yl)-5-[2-methyl-4-[[(2R)-1-methylazetidin-2-yl]methoxy]pyrazol-3-yl]pyrazolo[1,5-a]pyridin-2-amine;
N-(6-ethyl-2-methyl-pyrimidin-4-yl)-5-[2-methyl-4-[[(2S)-1-methylazetidin-2-yl]methoxy]pyrazol-3-yl]pyrazolo[1,5-a]pyridin-2-amine;
N-(5,6-dimethylpyrazin-2-yl)-5-[2-methyl-4-[[(2S)-1-methylazetidin-2-yl]methoxy]pyrazol-3-yl]pyrazolo[1,5-a]pyridin-2-amine;
N-(2,6-dimethylpyrimidin-4-yl)-5-[2-methyl-4-[[(2S)-1-methylazetidin-2-yl]methoxy]pyrazol-3-yl]pyrazolo[1,5-a]pyridin-2-amine;
5-[2-(difluoromethyl)-4-[[(2R)-1-methylazetidin-2-yl]methoxy]pyrazol-3-yl]-N-(2,6-dimethylpyrimidin-4-yl)pyrazolo[1,5-a]pyridin-2-amine;
5-[2-methyl-4-[[(2R)-1-methylazetidin-2-yl]methoxy]pyrazol-3-yl]-N-(5-methylpyridazin-3-yl)pyrazolo[1,5-a]pyridin-2-amine;
5-[2-methyl-4-[[(2R)-1-methylazetidin-2-yl]methoxy]pyrazol-3-yl]-N-(1-methylpyrazol-4-yl)pyrazolo[1,5-a]pyridin-2-amine;
2-methyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one;
N-(2-cyclopropyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(6-ethyl-2-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(2-methoxy-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine;
2-methyl-N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine;
N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine;
5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine;
N-(2-tert-butyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
2,4-dimethyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one;
N-[2-(cyclopropylmethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine;
N-(5-methoxypyrazin-2-yl)-5-[2-methyl-4-[[(2R)-1-methylazetidin-2-yl]methoxy]pyrazol-3-yl]pyrazolo[1,5-a]pyridin-2-amine;
N-[5-(difluoromethoxy)pyrazin-2-yl]-5-[2-methyl-4-[[(2R)-1-methylazetidin-2-yl]methoxy]pyrazol-3-yl]pyrazolo[1,5-a]pyridin-2-amine;
(1*S, 2*S)—N-[5-[2-methyl-4-[((R)-1-methylazetidin-2-yl)methoxy]pyrazol-3-yl]pyrazolo[1,5-a]pyridin-2-yl]-2-phenyl-cyclopropanecarboxamide;
(1*R,2*R)—N-[5-[2-methyl-4-[((R)1-methylazetidin-2-yl)methoxy]pyrazol-3-yl]pyrazolo[1,5-a]pyridin-2-yl]-2-phenyl-cyclopropanecarboxamide;
(1S,2R)—N-[5-[2-methyl-4-[[(2R)-1-methylazetidin-2-yl]methoxy]pyrazol-3-yl]pyrazolo[1,5-a]pyridin-2-yl]-2-phenyl-cyclopropanecarboxamide;
(1*S,2*R)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide;
5-[4-[[(2R)-1-ethylazetidin-2-yl]methoxy]-2-methyl-pyrazol-3-yl]-N-[5-(trifluoromethyl)pyrazin-2-yl]pyrazolo[1,5-a]pyridin-2-amine;
5-[4-[[(2R)-1-ethylazetidin-2-yl]methoxy]-2-methyl-pyrazol-3-yl]-N-[2-(trifluoromethyl)pyrimidin-4-yl]pyrazolo[1,5-a]pyridin-2-amine;
5-[4-[[(2R)-1-ethylazetidin-2-yl]methoxy]-2-methyl-pyrazol-3-yl]-N-[2-methyl-6-(trifluoromethyl)pyrimidin-4-yl]pyrazolo[1,5-a]pyridin-2-amine;
5-[4-[[(2R)-1-ethylazetidin-2-yl]methoxy]-2-methyl-pyrazol-3-yl]-N-(2-methoxypyrimidin-4-yl)pyrazolo[1,5-a]pyridin-2-amine;
5-[4-[[(2R)-1-ethylazetidin-2-yl]methoxy]-2-methyl-pyrazol-3-yl]-N-[6-(trifluoromethyl)pyrimidin-4-yl]pyrazolo[1,5-a]pyridin-2-amine;
N-(4,5-dimethylpyrimidin-2-yl)-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(5-methoxy-2-pyridyl)pyrazolo[1,5-a]pyridin-2-amine;
5-[4-[[(2R)-1-ethylazetidin-2-yl]methoxy]-2-methyl-pyrazol-3-yl]-N-(6-methoxypyrimidin-4-yl)pyrazolo[1,5-a]pyridin-2-amine;
5-[4-[[(2S)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
5-[4-[[(2S)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine;
6-[[5-[4-[[(2R)-1-ethylazetidin-2-yl]methoxy]-2-methyl-pyrazol-3-yl]pyrazolo[1,5-a]pyridin-2-yl]amino]-2-methyl-pyridazin-3-one;
N-(2-cyclopropyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine;
5-[4-[[(2R)-1-ethylazetidin-2-yl]methoxy]-2-methyl-pyrazol-3-yl]-N-(2-methoxy-6-methyl-pyrimidin-4-yl)pyrazolo[1,5-a]pyridin-2-amine;
N-[5-[4-[[(2R)-1-ethylazetidin-2-yl]methoxy]-2-methyl-pyrazol-3-yl]pyrazolo[1,5-a]pyridin-2-yl]-2-methyl-imidazo[1,2-b]pyridazin-6-amine;
N-[5-[4-[[(2R)-1-ethylazetidin-2-yl]methoxy]-2-methyl-pyrazol-3-yl]pyrazolo[1,5-a]pyridin-2-yl]imidazo[1,2-b]pyridazin-6-amine;
(R)-5-(4-((1-ethylazetidin-2-yl)methoxy)-1-methyl-1H-pyrazol-5-yl)-N-(5-fluoropyrimidin-2-yl)pyrazolo[1,5-a]pyridin-2-amine;
N-(2-tert-butyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine;
6-[[5-[4-[[(2R)-1-ethylazetidin-2-yl]methoxy]-2-methyl-pyrazol-3-yl]pyrazolo[1,5-a]pyridin-2-yl]amino]-2,4-dimethyl-pyridazin-3-one;
N-[2-(cyclopropylmethyl)-6-methyl-pyrimidin-4-yl]-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(5-methoxypyrazin-2-yl)pyrazolo[1,5-a]pyridin-2-amine;
N-[5-(difluoromethoxy)pyrazin-2-yl]-5-[4-[[(2R)-1-ethylazetidin-2-yl]methoxy]-2-methyl-pyrazol-3-yl]pyrazolo[1,5-a]pyridin-2-amine;
N-[5-[2-methyl-4-[[(3R)-pyrrolidin-3-yl]oxymethyl]pyrazol-3-yl]pyrazolo[1,5-a]pyridin-2-yl]cyclopropanecarboxamide;
N-[5-[2-methyl-4-[[(1S,5R)-3-oxa-9-azabicyclo[3.3.1]nonan-7-yl]oxymethyl]pyrazol-3-yl]pyrazolo[1,5-a]pyridin-2-yl]cyclopropanecarboxamide;
(RS)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[2-methyl-4-[[(1S,5R)-9-methyl-3-oxa-9-azabicyclo[3.3.1]nonan-7-yl]oxymethyl]pyrazol-3-yl]pyrazolo[1,5-a]pyridin-2-yl]cyclopropanecarboxamide;
N-[5-[2-methyl-4-[[3-(trifluoromethyl)azetidin-3-yl]oxymethyl]pyrazol-3-yl]pyrazolo[1,5-a]pyridin-2-yl]cyclopropanecarboxamide;
N-[5-[2-methyl-4-[[(2S,3R)-2-methylazetidin-3-yl]oxymethyl]pyrazol-3-yl]pyrazolo[1,5-a]pyridin-2-yl]cyclopropanecarboxamide;
N-[5-[4-[[(3R,4S)-4-fluoropyrrolidin-3-yl]oxymethyl]-2-methyl-pyrazol-3-yl]pyrazolo[1,5-a]pyridin-2-yl]cyclopropanecarboxamide;
(*R)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[4-[[(2S,3R)-1,2-dimethylazetidin-3-yl]oxymethyl]-2-methyl-pyrazol-3-yl]pyrazolo[1,5-a]pyridin-2-yl]cyclopropanecarboxamide;
N-[5-[2-methyl-4-[(3-methylazetidin-3-yl)oxymethyl]pyrazol-3-yl]pyrazolo[1,5-a]pyridin-2-yl]cyclopropanecarboxamide;
N-[5-[4-[(1,3-dimethylazetidin-3-yl)oxymethyl]-2-methyl-pyrazol-3-yl]pyrazolo[1,5-a]pyridin-2-yl]cyclopropanecarboxamide;
N-[5-[4-[(3-ethylazetidin-3-yl)oxymethyl]-2-methyl-pyrazol-3-yl]pyrazolo[1,5-a]pyridin-2-yl]cyclopropanecarboxamide;
(3*S,4*S)—N-[5-[2-methyl-4-[(4-methylpyrrolidin-3-yl)oxymethyl]pyrazol-3-yl]pyrazolo[1,5-a]pyridin-2-yl]cyclopropanecarboxamide;
(3*S,4*S)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide;
(3*R,4*R)—N-[5-[2-methyl-4-[(4-methylpyrrolidin-3-yl)oxymethyl]pyrazol-3-yl]pyrazolo[1,5-a]pyridin-2-yl]cyclopropanecarboxamide;
(3*R, 4*R)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide;
3-[[4-[2-(cyclopropanecarbonylamino)pyrazolo[1,5-a]pyridin-5-yl]-5-methyl-isoxazol-3-yl]oxymethyl]azetidine-3-carboxamide;
N-[5-[5-methyl-3-[(3-phenylazetidin-3-yl)methoxy]isoxazol-4-yl]pyrazolo[1,5-a]pyridin-2-yl]cyclopropanecarboxamide;
(rac-cis)-N-[5-[3-(4-methoxypyrrolidin-3-yl)oxy-5-methyl-isoxazol-4-yl]pyrazolo[1,5-a]pyridin-2-yl]cyclopropanecarboxamide;
N-[5-[5-methyl-3-[[(1R,4R)-5-oxa-2-azabicyclo[2.2.1]heptan-4-yl]methoxy]isoxazol-4-yl]pyrazolo[1,5-a]pyridin-2-yl]cyclopropanecarboxamide;
N-(5-(3-(((3*S,4*R)-4-methoxypyrrolidin-3-yl)oxy)-5-methylisoxazol-4-yl)pyrazolo[1,5-a]pyridin-2-yl)cyclopropanecarboxamide;
N-[5-[5-methyl-3-[[(1S,4S)-2-methyl-5-oxa-2-azabicyclo[2.2.1]heptan-4-yl]methoxy]isoxazol-4-yl]pyrazolo[1,5-a]pyridin-2-yl]cyclopropanecarboxamide;
N-[5-[3-[(2R)-2-amino-2-phenyl-ethoxy]-5-methyl-isoxazol-4-yl]pyrazolo[1,5-a]pyridin-2-yl]cyclopropanecarboxamide;
(RS)—N-[5-[3-[(3-methoxypyrrolidin-3-yl)methoxy]-5-methyl-isoxazol-4-yl]pyrazolo[1,5-a]pyridin-2-yl]cyclopropanecarboxamide;
N-[5-[3-[(4-methoxy-4-piperidyl)methoxy]-5-methyl-isoxazol-4-yl]pyrazolo[1,5-a]pyridin-2-yl]cyclopropanecarboxamide;
N-[5-[3-[(2S)-2-(dimethylamino)-2-phenyl-ethoxy]-5-methyl-isoxazol-4-yl]pyrazolo[1,5-a]pyridin-2-yl]cyclopropanecarboxamide;
(rac-cis)-N-[5-[3-(4-fluoropyrrolidin-3-yl)oxy-5-methyl-isoxazol-4-yl]pyrazolo[1,5-a]pyridin-2-yl]cyclopropanecarboxamide;
(*S)—N-(5-(3-((3-methoxypyrrolidin-3-yl)methoxy)-5-methylisoxazol-4-yl)pyrazolo[1,5-a]pyridin-2-yl)cyclopropanecarboxamide;
N-(5-(3-(((3*S,4*R)-4-fluoropyrrolidin-3-yl)oxy)-5-methylisoxazol-4-yl)pyrazolo[1,5-a]pyridin-2-yl)cyclopropanecarboxamide;
(rac-cis)-N-[5-[3-[(3-fluoro-4-piperidyl)oxy]-5-methyl-isoxazol-4-yl]pyrazolo[1,5-a]pyridin-2-yl]cyclopropanecarboxamide;
Exo-Rac-N-[5-[3-(2-azabicyclo[2.2.1]heptan-5-yloxy)-5-methyl-isoxazol-4-yl]pyrazolo[1,5-a]pyridin-2-yl]cyclopropanecarboxamide;
N-[5-[3-[[(2R)-1-(cyanomethyl)azetidin-2-yl]methoxy]-5-methyl-isoxazol-4-yl]pyrazolo[1,5-a]pyridin-2-yl]cyclopropanecarboxamide;
N-[5-[5-methyl-3-[[(2R)-4,6,6-trimethylmorpholin-2-yl]methoxy]isoxazol-4-yl]pyrazolo[1,5-a]pyridin-2-yl]cyclopropanecarboxamide;
N-(5-(3-(((3*S,4*R)-3-fluoropiperidin-4-yl)oxy)-5-methylisoxazol-4-yl)pyrazolo[1,5-a]pyridin-2-yl)cyclopropanecarboxamide;
N-(5-(3-(((3*R,4*S)-3-fluoropiperidin-4-yl)oxy)-5-methylisoxazol-4-yl)pyrazolo[1,5-a]pyridin-2-yl)cyclopropanecarboxamide;
N-(5-(3-(((1*S,4*S,5*R)-2-azabicyclo[2.2.1]heptan-5-yl)oxy)-5-methylisoxazol-4-yl)pyrazolo[1,5-a]pyridin-2-yl)cyclopropanecarboxamide;
(*R)—N-[5-[3-(9-azaspiro[3.5]nonan-6-yloxy)-5-methyl-isoxazol-4-yl]pyrazolo[1,5-a]pyridin-2-yl]cyclopropanecarboxamide;
(*R)—N-[5-[5-methyl-3-[(9-methyl-9-azaspiro[3.5]nonan-6-yl)oxy]isoxazol-4-yl]pyrazolo[1,5-a]pyridin-2-yl]cyclopropanecarboxamide;
N-[5-[3-[(2S)-2-amino-2-(2-fluorophenyl)ethoxy]-5-methyl-isoxazol-4-yl]pyrazolo[1,5-a]pyridin-2-yl]cyclopropanecarboxamide;
N-[5-[3-[(2R)-2-amino-2-(2-fluorophenyl)ethoxy]-5-methyl-isoxazol-4-yl]pyrazolo[1,5-a]pyridin-2-yl]cyclopropanecarboxamide;
N-[5-[3-[(2R)-2-(dimethylamino)-2-(2-fluorophenyl)ethoxy]-5-methyl-isoxazol-4-yl]pyrazolo[1,5-a]pyridin-2-yl]cyclopropanecarboxamide;
N-[5-[3-[(2S)-2-amino-2-(2-pyridyl)ethoxy]-5-methyl-isoxazol-4-yl]pyrazolo[1,5-a]pyridin-2-yl]cyclopropanecarboxamide;
N-[5-[5-methyl-3-[(2S)-2-(methylamino)-2-(2-pyridyl)ethoxy]isoxazol-4-yl]pyrazolo[1,5-a]pyridin-2-yl]cyclopropanecarboxamide;
N-[5-[3-[(2S)-2-amino-2-phenyl-propoxy]-5-methyl-isoxazol-4-yl]pyrazolo[1,5-a]pyridin-2-yl]cyclopropanecarboxamide;
N-[5-[3-[(2S)-2-(dimethylamino)-2-phenyl-propoxy]-5-methyl-isoxazol-4-yl]pyrazolo[1,5-a]pyridin-2-yl]cyclopropanecarboxamide;
(3R, 3aS, 7aR)—N-[5-[3-(1,2,3,3a,5,6,7,7a-octahydropyrano[3,2-b]pyrrol-3-yloxy)-5-methyl-isoxazol-4-yl]pyrazolo[1,5-a]pyridin-2-yl]cyclopropanecarboxamide;
N-(5-(5-methyl-3-(((3*S,3a*R,7a*S)-octahydropyrano[3,2-b]pyrrol-3-yl)oxy)isoxazol-4-yl)pyrazolo[1,5-a]pyridin-2-yl)cyclopropanecarboxamide;
N-[5-[2-methyl-4-[[(3R)-pyrrolidin-3-yl]methoxy]pyrazol-3-yl]pyrazolo[1,5-a]pyridin-2-yl]cyclopropanecarboxamide;
N-[5-[2-methyl-4-[[(2S)-morpholin-2-yl]methoxy]pyrazol-3-yl]pyrazolo[1,5-a]pyridin-2-yl]cyclopropanecarboxamide;
N-[5-[4-[(1-aminocyclobutyl)methoxy]-2-methyl-pyrazol-3-yl]pyrazolo[1,5-a]pyridin-2-yl]cyclopropanecarboxamide;
(rac-cis)-N-[5-[4-(4-methoxypyrrolidin-3-yl)oxy-2-methyl-pyrazol-3-yl]pyrazolo[1,5-a]pyridin-2-yl]cyclopropanecarboxamide;
N-[5-[2-methyl-4-[(3S,5R)-5-methylpyrrolidin-3-yl]oxy-pyrazol-3-yl]pyrazolo[1,5-a]pyridin-2-yl]cyclopropanecarboxamide;
N-[5-[2-methyl-4-[[(1R,4R)-5-oxa-2-azabicyclo[2.2.1]heptan-4-yl]methoxy]pyrazol-3-yl]pyrazolo[1,5-a]pyridin-2-yl]cyclopropanecarboxamide;
N-[5-[4-[(2S)-2-amino-2-phenyl-ethoxy]-2-methyl-pyrazol-3-yl]pyrazolo[1,5-a]pyridin-2-yl]cyclopropanecarboxamide;
N-[5-[2-methyl-4-[(3S)-pyrrolidin-3-yl]oxy-pyrazol-3-yl]pyrazolo[1,5-a]pyridin-2-yl]cyclopropanecarboxamide;
N-[5-[4-[(2R)-2-amino-2-phenyl-ethoxy]-2-methyl-pyrazol-3-yl]pyrazolo[1,5-a]pyridin-2-yl]cyclopropanecarboxamide;
(3*R, 4*R)—N-[5-[2-methyl-4-(4-methylpyrrolidin-3-yl)oxy-pyrazol-3-yl]pyrazolo[1,5-a]pyridin-2-yl]cyclopropanecarboxamide;
N-[5-[2-methyl-4-[[(2R)-1-methylpyrrolidin-2-yl]methoxy]pyrazol-3-yl]pyrazolo[1,5-a]pyridin-2-yl]cyclopropanecarboxamide;
(RS)—N-[5-[4-(5-azaspiro[2.3]hexan-6-ylmethoxy)-2-methyl-pyrazol-3-yl]pyrazolo[1,5-a]pyridin-2-yl]cyclopropanecarboxamide;
N-[5-[4-[[(2R)-1-(2-fluoroethyl)azetidin-2-yl]methoxy]-2-methyl-pyrazol-3-yl]pyrazolo[1,5-a]pyridin-2-yl]cyclopropanecarboxamide;
N-[5-[4-[(3R,4S)-4-fluoropyrrolidin-3-yl]oxy-2-methyl-pyrazol-3-yl]pyrazolo[1,5-a]pyridin-2-yl]cyclopropanecarboxamide;
N-[5-[4-[[(2R)-1-(2-methoxyethyl)azetidin-2-yl]methoxy]-2-methyl-pyrazol-3-yl]pyrazolo[1,5-a]pyridin-2-yl]cyclopropanecarboxamide;
N-[5-[4-[[(2R)-1-isobutylazetidin-2-yl]methoxy]-2-methyl-pyrazol-3-yl]pyrazolo[1,5-a]pyridin-2-yl]cyclopropanecarboxamide;
N-[5-[4-[[(2R)-1-(2-hydroxy-2-methyl-propyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-propylazetidin-2-yl]methoxy]pyrazol-3-yl]pyrazolo[1,5-a]pyridin-2-yl]cyclopropanecarboxamide;
N-[5-[4-[[(2R)-1-(cyclopropylmethyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2S)-1-(trideuteriomethyl)azetidin-2-yl]methoxy]pyrazol-3-yl]pyrazolo[1,5-a]pyridin-2-yl]cyclopropanecarboxamide;
N-[5-[2-methyl-4-[[(2R)-1-(trideuteriomethyl)pyrrolidin-2-yl]methoxy]pyrazol-3-yl]pyrazolo[1,5-a]pyridin-2-yl]cyclopropanecarboxamide;
N-[5-[2-methyl-4-[[(2R)-1-(trideuteriomethyl)azetidin-2-yl]methoxy]pyrazol-3-yl]pyrazolo[1,5-a]pyridin-2-yl]cyclopropanecarboxamide;
N-[5-[4-[[(2R)-1-acetonylazetidin-2-yl]methoxy]-2-methyl-pyrazol-3-yl]pyrazolo[1,5-a]pyridin-2-yl]cyclopropanecarboxamide;
N-[5-[4-[[(2R)-1-[(2R)-2-hydroxypropyl]azetidin-2-yl]methoxy]-2-methyl-pyrazol-3-yl]pyrazolo[1,5-a]pyridin-2-yl]cyclopropanecarboxamide;
N-[5-[4-[[(2R)-1-[(2S)-2-hydroxypropyl]azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-(1,1,2,2,2-pentadeuterioethyl)azetidin-2-yl]methoxy]pyrazol-3-yl]pyrazolo[1,5-a]pyridin-2-yl]cyclopropanecarboxamide;
N-[5-[4-[[(2R)-1-(2-cyano-2-methyl-propyl)azetidin-2-yl]methoxy]-2-methyl-pyrazol-3-yl]pyrazolo[1,5-a]pyridin-2-yl]cyclopropanecarboxamide;
N-[5-[4-[[(2R,5R)-1,5-dimethylpyrrolidin-2-yl]methoxy]-2-methyl-pyrazol-3-yl]pyrazolo[1,5-a]pyridin-2-yl]cyclopropanecarboxamide;
N-[5-[2-methyl-4-[[(2R,4S)-1-methyl-4-phenyl-pyrrolidin-2-yl]methoxy]pyrazol-3-yl]pyrazolo[1,5-a]pyridin-2-yl]cyclopropanecarboxamide;
N-[5-[2-methyl-4-[[(2R,4R)-1-methyl-4-phenyl-pyrrolidin-2-yl]methoxy]pyrazol-3-yl]pyrazolo[1,5-a]pyridin-2-yl]cyclopropanecarboxamide;
N-(5-(1-methyl-4-(((R)-1-(((R)-tetrahydrofuran-2-yl)methyl)azetidin-2-yl)methoxy)-1H-pyrazol-5-yl)pyrazolo[1,5-a]pyridin-2-yl)cyclopropanecarboxamide;
5-[4-[[(2R)-azetidin-2-yl]methoxy]-2-(difluoromethyl)pyrazol-3-yl]-N-(2,6-dimethylpyrimidin-4-yl)pyrazolo[1,5-a]pyridin-2-amine;
(*R)-5-(1-(difluoromethyl)-4-((3-methoxypyrrolidin-3-yl)methoxy)-1H-pyrazol-5-yl)-N-(2,6-dimethylpyrimidin-4-yl)pyrazolo[1,5-a]pyridin-2-amine;
(*R)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine;
(*S)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(5-methylpyrazin-2-yl)pyrazolo[1,5-a]pyridin-2-amine;
5-[2-methyl-4-[(3S)-1-methylpyrrolidin-3-yl]oxy-pyrazol-3-yl]-N-(6-methylpyrimidin-4-yl)pyrazolo[1,5-a]pyridin-2-amine;
5-[2-methyl-4-[(3S)-1-methylpyrrolidin-3-yl]oxy-pyrazol-3-yl]-N-[6-(trifluoromethyl)-2-pyridyl]pyrazolo[1,5-a]pyridin-2-amine;
N-(2,6-dimethylpyrimidin-4-yl)-5-[2-methyl-4-[(3 S)-1-methylpyrrolidin-3-yl]oxy-pyrazol-3-yl]pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]pyrazolo[1,5-a]pyrimidin-5-amine;
N-(6,7-dihydro-5H-cyclopenta[c]pyridazin-3-yl)-5-[2-methyl-4-[(3S)-1-methylpyrrolidin-3-yl]oxy-pyrazol-3-yl]pyrazolo[1,5-a]pyridin-2-amine;
N-(2-isopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(6-isopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(5,6-dimethylpyridazin-3-yl)-5-[2-methyl-4-[(3S)-1-methylpyrrolidin-3-yl]oxy-pyrazol-3-yl]pyrazolo[1,5-a]pyridin-2-amine;
5-[4-[[(2R)-azetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
N-(2,6-dimethylpyrimidin-4-yl)-5-[4-[[(2R)-1-(2-fluoroethyl)azetidin-2-yl]methoxy]-2-methyl-pyrazol-3-yl]pyrazolo[1,5-a]pyridin-2-amine;
5-[4-[[(2R)-azetidin-2-yl]methoxy]-2-methyl-pyrazol-3-yl]-N-(5,6-dimethylpyridazin-3-yl)pyrazolo[1,5-a]pyridin-2-amine;
N-(5,6-dimethylpyridazin-3-yl)-5-[4-[[(2R)-1-(2-fluoroethyl)azetidin-2-yl]methoxy]-2-methyl-pyrazol-3-yl]pyrazolo[1,5-a]pyridin-2-amine;
5-[4-[[(2R)-1-(2-fluoroethyl)azetidin-2-yl]methoxy]-2-methyl-pyrazol-3-yl]-N-(6-methylpyrimidin-4-yl)pyrazolo[1,5-a]pyridin-2-amine;
5-[4-[[(2R)-1-(2-fluoroethyl)azetidin-2-yl]methoxy]-2-methyl-pyrazol-3-yl]-N-(6-methylpyrazin-2-yl)pyrazolo[1,5-a]pyridin-2-amine;
5-[4-[[(2R)-1-(2-fluoroethyl)azetidin-2-yl]methoxy]-2-methyl-pyrazol-3-yl]-N-(5-methylpyrazin-2-yl)pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]-7,8-dihydro-5H-pyrano[4,3-b]pyridin-2-amine;
N-(2-cyclopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(2,6-dimethylpyrimidin-4-yl)-5-[2-methyl-4-[[(2R)-1-methylpyrrolidin-2-yl]methoxy]pyrazol-3-yl]pyrazolo[1,5-a]pyridin-2-amine;
5-[2-methyl-4-[[(2R)-1-methylpyrrolidin-2-yl]methoxy]pyrazol-3-yl]-N-[6-methyl-2-(trifluoromethyl)pyrimidin-4-yl]pyrazolo[1,5-a]pyridin-2-amine;
N-(6-cyclopropylpyridazin-3-yl)-5-[2-methyl-4-[[(2R)-1-methylpyrrolidin-2-yl]methoxy]pyrazol-3-yl]pyrazolo[1,5-a]pyridin-2-amine;
5-[2-methyl-4-[[(2R)-1-methylpyrrolidin-2-yl]methoxy]pyrazol-3-yl]-N-(6-methylpyrimidin-4-yl)pyrazolo[1,5-a]pyridin-2-amine;
5-[2-methyl-4-[[(2R)-1-methylpyrrolidin-2-yl]methoxy]pyrazol-3-yl]-N-(2-methylpyrimidin-4-yl)pyrazolo[1,5-a]pyridin-2-amine;
5-[2-methyl-4-[[(2R)-1-methylpyrrolidin-2-yl]methoxy]pyrazol-3-yl]-N-(6-methylpyridazin-3-yl)pyrazolo[1,5-a]pyridin-2-amine;
5-[4-(3-azabicyclo[2.1.1]hexan-4-ylmethoxy)-2-methyl-pyrazol-3-yl]-N-(2,6-dimethylpyrimidin-4-yl)pyrazolo[1,5-a]pyridin-2-amine;
N-(2,6-dimethylpyrimidin-4-yl)-5-[4-[[(2R)-1-ethylpyrrolidin-2-yl]methoxy]-2-methyl-pyrazol-3-yl]pyrazolo[1,5-a]pyridin-2-amine;
N-(2,6-dimethylpyrimidin-4-yl)-5-[2-methyl-4-[[(2R)-1-(trideuteriomethyl)azetidin-2-yl]methoxy]pyrazol-3-yl]pyrazolo[1,5-a]pyridin-2-amine;
5-[4-[[(2R)-azetidin-2-yl]methoxy]-2-methyl-pyrazol-3-yl]-N-(6-pyrazol-1-ylpyrazin-2-yl)pyrazolo[1,5-a]pyridin-2-amine;
(2S)-1-[(2R)-2-[[5-[2-[(2,6-dimethylpyrimidin-4-yl)amino]pyrazolo[1,5-a]pyridin-5-yl]-1-methyl-pyrazol-4-yl]oxymethyl]azetidin-1-yl]propan-2-ol;
N-(2,6-dimethylpyrimidin-4-yl)-5-[2-methyl-4-[[(2R)-1-(1,1,2,2,2-pentadeuterioethyl)pyrrolidin-2-yl]methoxy]pyrazol-3-yl]pyrazolo[1,5-a]pyridin-2-amine;
N-(2,6-dimethylpyrimidin-4-yl)-5-[2-methyl-4-[[(2R)-1-(1,1,2,2,2-pentadeuterioethyl)azetidin-2-yl]methoxy]pyrazol-3-yl]pyrazolo[1,5-a]pyridin-2-amine;
(2*R, 3*S)—N-(2,6-dimethylpyrimidin-4-yl)-5-[2-methyl-4-[(2-methyltetrahydrofuran-3-yl)methoxy]pyrazol-3-yl]pyrazolo[1,5-a]pyridin-2-amine;
(2S)-1-[(2R)-2-[[1-methyl-5-[2-[(5-methylpyrazin-2-yl)amino]pyrazolo[1,5-a]pyridin-5-yl]pyrazol-4-yl]oxymethyl]azetidin-1-yl]propan-2-ol;
(2S)-1-[(2R)-2-[[5-[2-[(5,6-dimethylpyrazin-2-yl)amino]pyrazolo[1,5-a]pyridin-5-yl]-1-methyl-pyrazol-4-yl]oxymethyl]azetidin-1-yl]propan-2-ol;
N-(2,6-dimethylpyrimidin-4-yl)-5-[2-methyl-4-[(2S,3R)-2-methylpyrrolidin-3-yl]oxy-pyrazol-3-yl]pyrazolo[1,5-a]pyridin-2-amine;
(*S)—N-(2,6-dimethylpyrimidin-4-yl)-5-[4-(6,9-dioxaspiro[4.4]nonan-8-ylmethoxy)-2-methyl-pyrazol-3-yl]pyrazolo[1,5-a]pyridin-2-amine;
(2R,3S)-2-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol;
(1S)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol;
(S)-1-cyclobutyl-2-[5-[2-[(2,6-dimethylpyrimidin-4-yl)amino]pyrazolo[1,5-a]pyridin-5-yl]-1-methyl-pyrazol-4-yl]oxy-ethanol;
(2S)-1-[(2R)-2-[[1-methyl-5-[2-[[2-methyl-6-(trifluoromethyl)pyrimidin-4-yl]amino]pyrazolo[1,5-a]pyridin-5-yl]pyrazol-4-yl]oxymethyl]azetidin-1-yl]propan-2-ol;
(*S)-2-cyclopropyl-1-[5-[2-[(2,6-dimethylpyrimidin-4-yl)amino]pyrazolo[1,5-a]pyridin-5-yl]-1-methyl-pyrazol-4-yl]oxy-propan-2-ol;
N-(2,6-dimethylpyrimidin-4-yl)-5-[4-[[(2R,4S)-1,4-dimethylpyrrolidin-2-yl]methoxy]-2-methyl-pyrazol-3-yl]pyrazolo[1,5-a]pyridin-2-amine;
(*S)-1-(3-bicyclo[1.1.1]pentanyl)-2-[5-[2-[(2,6-dimethylpyrimidin-4-yl)amino]pyrazolo[1,5-a]pyridin-5-yl]-1-methyl-pyrazol-4-yl]oxy-ethanol;
(*R)—N-(2,6-dimethylpyrimidin-4-yl)-5-[4-[(5,5-dimethyltetrahydrofuran-3-yl)methoxy]-2-methyl-pyrazol-3-yl]pyrazolo[1,5-a]pyridin-2-amine;
5-[2-methyl-4-[[(2S)-1-methylazetidin-2-yl]methoxy]pyrazol-3-yl]-N-[6-methyl-2-(trifluoromethyl)pyrimidin-4-yl]pyrazolo[1,5-a]pyridin-2-amine;
5-[4-[2-amino-2-(1-methylcyclopropyl)ethoxy]-2-methyl-pyrazol-3-yl]-N-(2,6-dimethylpyrimidin-4-yl)pyrazolo[1,5-a]pyridin-2-amine;
5-[4-[2-amino-2-(3-bicyclo[1.1.1]pentanyl)ethoxy]-2-methyl-pyrazol-3-yl]-N-(2,6-dimethylpyrimidin-4-yl)pyrazolo[1,5-a]pyridin-2-amine;
methyl 5-[[5-[4-[[(2R)-1-ethylazetidin-2-yl]methoxy]-2-methyl-pyrazol-3-yl]pyrazolo[1,5-a]pyridin-2-yl]amino]pyrazine-2-carboxylate;
N-[5-[2-ethyl-4-[[(3R)-pyrrolidin-3-yl]methoxy]pyrazol-3-yl]pyrazolo[1,5-a]pyridin-2-yl]cyclopropanecarboxamide;
N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide;
N-[5-[4-[(1,3-dimethylazetidin-3-yl)methoxy]-2-methyl-pyrazol-3-yl]pyrazolo[1,5-a]pyridin-2-yl]cyclopropanecarboxamide;
N-[5-[2-methyl-4-[[(2R)-1-(2,2,2-trifluoroethyl)azetidin-2-yl]methoxy]pyrazol-3-yl]pyrazolo[1,5-a]pyridin-2-yl]cyclopropanecarboxamide;
N-[5-[5-methyl-3-(4-piperidyloxy)isoxazol-4-yl]pyrazolo[1,5-a]pyridin-2-yl]cyclopropanecarboxamide;
*S, *R—N-[5-[3-(4-methoxy-1-methyl-pyrrolidin-3-yl)oxy-5-methyl-isoxazol-4-yl]pyrazolo[1,5-a]pyridin-2-yl]cyclopropanecarboxamide;
5-[3-[(3-methoxyazetidin-3-yl)methoxy]-5-methyl-isoxazol-4-yl]-N-pyrazin-2-yl-pyrazolo[1,5-a]pyridin-2-amine;
N-[5-[3-[(3-aminocyclobutyl)methoxy]-5-methyl-isoxazol-4-yl]pyrazolo[1,5-a]pyridin-2-yl]cyclopropanecarboxamide;
5-[3-[[(2R)-azetidin-2-yl]methoxy]-5-methyl-isoxazol-4-yl]-N-(2,6-dimethylpyrimidin-4-yl)pyrazolo[1,5-a]pyridin-2-amine;
N-[5-[5-methyl-3-[[(2S)-1,3,3-trimethylazetidin-2-yl]methoxy]isoxazol-4-yl]pyrazolo[1,5-a]pyridin-2-yl]cyclopropanecarboxamide;
5-[5-methyl-3-[(3S,5R)-5-methylpyrrolidin-3-yl]oxy-isoxazol-4-yl]-N-(5-methylpyrazin-2-yl)pyrazolo[1,5-a]pyridin-2-amine;
5-[5-methyl-3-[[(2R)-1-methylazetidin-2-yl]methoxy]isoxazol-4-yl]-N-(5-methylpyrazin-2-yl)pyrazolo[1,5-a]pyridin-2-amine;
N-[5-[3-[(2S)-2-(dimethylamino)-2-(2-fluorophenyl)ethoxy]-5-methyl-isoxazol-4-yl]pyrazolo[1,5-a]pyridin-2-yl]cyclopropanecarboxamide;
N-[5-[3-[(2S)-2-(dimethylamino)-2-(2-pyridyl)ethoxy]-5-methyl-isoxazol-4-yl]pyrazolo[1,5-a]pyridin-2-yl]cyclopropanecarboxamide;
N-[5-[3-[(2R)-2-amino-2-phenyl-propoxy]-5-methyl-isoxazol-4-yl]pyrazolo[1,5-a]pyridin-2-yl]cyclopropanecarboxamide;
5-[3-[(3S,5S)-5-(difluoromethyl)pyrrolidin-3-yl]oxy-5-methyl-isoxazol-4-yl]-N-(2,6-dimethylpyrimidin-4-yl)pyrazolo[1,5-a]pyridin-2-amine;
N-[5-[3-[(2S)-2-amino-2-cyclopropyl-ethoxy]-5-methyl-isoxazol-4-yl]pyrazolo[1,5-a]pyridin-2-yl]cyclopropanecarboxamide;
N-[6-[5-methyl-3-[[(2R)-1-methylazetidin-2-yl]methoxy]isoxazol-4-yl]imidazo[1,2-a]pyridin-2-yl]cyclopropanecarboxamide;
N-[5-[5-methyl-3-[(3S)-pyrrolidin-3-yl]oxy-isothiazol-4-yl]pyrazolo[1,5-a]pyridin-2-yl]cyclopropanecarboxamide;
N-[5-[5-methyl-3-[[(2R)-morpholin-2-yl]methoxy]isothiazol-4-yl]pyrazolo[1,5-a]pyridin-2-yl]cyclopropanecarboxamide;
N-[5-[5-methyl-3-[[(2R)-4-methylmorpholin-2-yl]methoxy]isothiazol-4-yl]pyrazolo[1,5-a]pyridin-2-yl]cyclopropanecarboxamide;
N-[5-[2-methyl-4-[[(2R)-4-methylmorpholin-2-yl]methoxy]pyrazol-3-yl]pyrazolo[1,5-a]pyridin-2-yl]cyclopropanecarboxamide;
N-[5-[4-[(3-aminocyclobutyl)methoxy]-2-methyl-pyrazol-3-yl]pyrazolo[1,5-a]pyridin-2-yl]cyclopropanecarboxamide;
N-[5-[2-methyl-4-[(3R)-pyrrolidin-3-yl]oxy-pyrazol-3-yl]pyrazolo[1,5-a]pyridin-2-yl]cyclopropanecarboxamide;
N-[5-[4-[(3S,5R)-1,5-dimethylpyrrolidin-3-yl]oxy-2-methyl-pyrazol-3-yl]pyrazolo[1,5-a]pyridin-2-yl]cyclopropanecarboxamide;
(2*S, 3*R)—N-[5-[4-[(1,2-dimethylazetidin-3-yl)methoxy]-2-methyl-pyrazol-3-yl]pyrazolo[1,5-a]pyridin-2-yl]cyclopropanecarboxamide;
N-[5-[2-methyl-4-[(3S,5S)-5-methylpyrrolidin-3-yl]oxy-pyrazol-3-yl]pyrazolo[1,5-a]pyridin-2-yl]cyclopropanecarboxamide;
5-[2-methyl-4-[(3S)-1-methylpyrrolidin-3-yl]oxy-pyrazol-3-yl]-N-[5-(trifluoromethyl)-2-pyridyl]pyrazolo[1,5-a]pyridin-2-amine;
5-[2-methyl-4-[(3S)-1-methylpyrrolidin-3-yl]oxy-pyrazol-3-yl]-N-[4-(trifluoromethyl)-2-pyridyl]pyrazolo[1,5-a]pyridin-2-amine;
N-[5-[2-methyl-4-[[(2S,3 S)-3-methylazetidin-2-yl]methoxy]pyrazol-3-yl]pyrazolo[1,5-a]pyridin-2-yl]cyclopropanecarboxamide;
N-[5-[2-methyl-4-[(3S)-1-methylpyrrolidin-3-yl]oxy-pyrazol-3-yl]pyrazolo[1,5-a]pyridin-2-yl]cinnolin-3-amine;
N-[5-[2-methyl-4-[(3S)-1-methylpyrrolidin-3-yl]oxy-pyrazol-3-yl]pyrazolo[1,5-a]pyridin-2-yl]quinoxalin-2-amine;
5-[2-methyl-4-[(3S)-1-methylpyrrolidin-3-yl]oxy-pyrazol-3-yl]-N-pyrazolo[1,5-a]pyridin-2-yl-pyrazolo[1,5-a]pyridin-2-amine;
N-[5-[4-[[(3R,4R)-4-hydroxypyrrolidin-3-yl]methoxy]-2-methyl-pyrazol-3-yl]pyrazolo[1,5-a]pyridin-2-yl]cyclopropanecarboxamide;
(R)—N-(5-(4-(azetidin-2-ylmethoxy)-1-methyl-1H-pyrazol-5-yl)pyrazolo[1,5-a]pyridin-2-yl)cyclopropanecarboxamide;
N-[5-[4-[[(2R)-1-cyclopropylazetidin-2-yl]methoxy]-2-methyl-pyrazol-3-yl]pyrazolo[1,5-a]pyridin-2-yl]cyclopropanecarboxamide;
N-[5-[4-[(2S)-2-amino-3,3-dimethyl-butoxy]-2-methyl-pyrazol-3-yl]pyrazolo[1,5-a]pyridin-2-yl]cyclopropanecarboxamide;
N-(5,6-dimethylpyrazin-2-yl)-5-[2-methyl-4-[(3S)-1-methylpyrrolidin-3-yl]oxy-pyrazol-3-yl]pyrazolo[1,5-a]pyridin-2-amine;
N-[5-[4-[[(2R)-1-(2-fluoroethyl)pyrrolidin-2-yl]methoxy]-2-methyl-pyrazol-3-yl]pyrazolo[1,5-a]pyridin-2-yl]cyclopropanecarboxamide;
N-[5-[4-[[(3R,4R)-4-methoxypyrrolidin-3-yl]methoxy]-2-methyl-pyrazol-3-yl]pyrazolo[1,5-a]pyridin-2-yl]cyclopropanecarboxamide;
(1*S, 2*S, 4*R)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine;
(*S)—N-[5-[2-methyl-4-[[1-(tetrahydrofuran-2-ylmethyl)azetidin-2-yl]methoxy]pyrazol-3-yl]pyrazolo[1,5-a]pyridin-2-yl]cyclopropanecarboxamide;
N-(2,6-dimethylpyrimidin-4-yl)-5-[4-[(2S,3R)-1,2-dimethylpyrrolidin-3-yl]oxy-2-methyl-pyrazol-3-yl]pyrazolo[1,5-a]pyridin-2-amine;
N-(2,6-dimethylpyrimidin-4-yl)-5-[2-(trideuteriomethyl)-4-[[(2R)-1-(trideuteriomethyl)azetidin-2-yl]methoxy]pyrazol-3-yl]pyrazolo[1,5-a]pyridin-2-amine;
(1*R)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol;
*S-1-methylcyclopropyl-2-[5-[2-[(2,6-dimethylpyrimidin-4-yl)amino]pyrazolo[1,5-a]pyridin-5-yl]-1-methyl-pyrazol-4-yl]oxy-ethanol;
*S—N-(2,6-dimethylpyrimidin-4-yl)-5-[2-methyl-4-(7-oxaspiro[3.4]octan-6-ylmethoxy)pyrazol-3-yl]pyrazolo[1,5-a]pyridin-2-amine;
N-(5,6-dimethylpyrazin-2-yl)-5-[4-[[(2S)-1-ethylazetidin-2-yl]methoxy]-2-methyl-pyrazol-3-yl]pyrazolo[1,5-a]pyridin-2-amine;
N-[5-[4-(azetidin-3-yloxymethyl)-2-methyl-pyrazol-3-yl]pyrazolo[1,5-a]pyridin-2-yl]cyclopropanecarboxamide;
N-[5-[2-methyl-4-[(1-methylazetidin-3-yl)oxymethyl]pyrazol-3-yl]pyrazolo[1,5-a]pyridin-2-yl]cyclopropanecarboxamide;
N-[5-[2-methyl-4-[[(3R)-1-methylpyrrolidin-3-yl]oxymethyl]pyrazol-3-yl]pyrazolo[1,5-a]pyridin-2-yl]cyclopropanecarboxamide;
N-[5-[2-methyl-4-[[(3S)-pyrrolidin-3-yl]oxymethyl]pyrazol-3-yl]pyrazolo[1,5-a]pyridin-2-yl]cyclopropanecarboxamide;
Rac-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
Trans-*R, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
Trans-*S, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
(rac-cis)-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS—*R, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS—*S, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS-Rac-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*R, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
Trans-*S, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
(rac-trans)-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*S, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*R, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Rac-CIS-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
CIS—*R, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
CIS—*R, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
CIS—*S, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
CIS—*S, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
N-(5-tert-butylpyrazin-2-yl)-5-[2-methyl-4-[[(2R)-1-methylazetidin-2-yl]methoxy]pyrazol-3-yl]pyrazolo[1,5-a]pyridin-2-amine;
N-[5-[5-methyl-3-[(3S)-1-methylpyrrolidin-3-yl]oxy-isoxazol-4-yl]pyrazolo[1,5-a]pyridin-2-yl]cyclopropanecarboxamide;
N-[5-[5-methyl-3-[[(3R)-3-piperidyl]methoxy]isoxazol-4-yl]pyrazolo[1,5-a]pyridin-2-yl]cyclopropanecarboxamide;
N-[5-[5-methyl-3-[(3S)-1-(trideuteriomethyl)pyrrolidin-3-yl]oxy-isoxazol-4-yl]pyrazolo[1,5-a]pyridin-2-yl]cyclopropanecarboxamide;
(3*R,4*S)—N-[5-[3-(4-methoxypyrrolidin-3-yl)oxy-5-methyl-isoxazol-4-yl]pyrazolo[1,5-a]pyridin-2-yl]cyclopropanecarboxamide;
N-[5-[3-(3-aminocyclobutoxy)-5-methyl-isoxazol-4-yl]pyrazolo[1,5-a]pyridin-2-yl]cyclopropanecarboxamide;
N-[5-[3-[(3-aminocyclobutyl)methoxy]-5-methyl-isoxazol-4-yl]pyrazolo[1,5-a]pyridin-2-yl]cyclopropanecarboxamide;
N-[5-[5-methyl-3-[[(2R)-1-methylpyrrolidin-2-yl]methoxy]isoxazol-4-yl]pyrazolo[1,5-a]pyridin-2-yl]cyclopropanecarboxamide;
N-[5-[3-[(3S,5S)-5-(difluoromethyl)pyrrolidin-3-yl]oxy-5-methyl-isoxazol-4-yl]pyrazolo[1,5-a]pyridin-2-yl]cyclopropanecarboxamide;
N-(5,6-dimethylpyridazin-3-yl)-5-[3-[(3S,5S)-5-(methoxymethyl)pyrrolidin-3-yl]oxy-5-methyl-isoxazol-4-yl]pyrazolo[1,5-a]pyridin-2-amine;
N-(5,6-dimethylpyridazin-3-yl)-5-[3-[(3S,5S)-5-(methoxymethyl)-1-methyl-pyrrolidin-3-yl]oxy-5-methyl-isoxazol-4-yl]pyrazolo[1,5-a]pyridin-2-amine;
5-[4-[[(2R)-azetidin-2-yl]methoxy]-2-methyl-pyrazol-3-yl]-N-(6-methylpyridazin-3-yl)pyrazolo[1,5-a]pyridin-2-amine;
5-[4-[[(2R)-azetidin-2-yl]methoxy]-2-methyl-pyrazol-3-yl]-N-(5-methylpyrazin-2-yl)pyrazolo[1,5-a]pyridin-2-amine;
5-[4-[[(2R)-azetidin-2-yl]methoxy]-2-methyl-pyrazol-3-yl]-N-(6-cyclopropylpyridazin-3-yl)pyrazolo[1,5-a]pyridin-2-amine;
N-(5-fluoropyrimidin-2-yl)-5-[2-methyl-4-[[(2R)-1-methylazetidin-2-yl]methoxy]pyrazol-3-yl]pyrazolo[1,5-a]pyridin-2-amine;
N-[5-[2-methyl-4-[[(3R)-1-methylpyrrolidin-3-yl]methoxy]pyrazol-3-yl]pyrazolo[1,5-a]pyridin-2-yl]cyclopropanecarboxamide;
N-[5-[4-(azetidin-3-yloxy)-2-methyl-pyrazol-3-yl]pyrazolo[1,5-a]pyridin-2-yl]cyclopropanecarboxamide;
N-[5-[2-ethyl-4-[[(3R)-1-methylpyrrolidin-3-yl]methoxy]pyrazol-3-yl]pyrazolo[1,5-a]pyridin-2-yl]cyclopropanecarboxamide;
N-[5-[2-methyl-4-(1-methylazetidin-3-yl)oxy-pyrazol-3-yl]pyrazolo[1,5-a]pyridin-2-yl]cyclopropanecarboxamide;
N-[5-[4-[(3-aminocyclobutyl)methoxy]-2-methyl-pyrazol-3-yl]pyrazolo[1,5-a]pyridin-2-yl]cyclopropanecarboxamide;
(*R)—N-[5-[2-methyl-4-(3,3,3-trifluoro-2-hydroxy-propoxy)pyrazol-3-yl]pyrazolo[1,5-a]pyridin-2-yl]cyclopropanecarboxamide;
N-[5-[4-[(3-aminooxetan-3-yl)methoxy]-2-methyl-pyrazol-3-yl]pyrazolo[1,5-a]pyridin-2-yl]cyclopropanecarboxamide;
N-[5-[4-[(2R)-2-hydroxy-2-phenyl-ethoxy]-2-methyl-pyrazol-3-yl]pyrazolo[1,5-a]pyridin-2-yl]cyclopropanecarboxamide;
N-[5-[4-(4-hydroxycyclohexoxy)-2-methyl-pyrazol-3-yl]pyrazolo[1,5-a]pyridin-2-yl]cyclopropanecarboxamide;
N-[5-[4-[(3-fluoro-1-methyl-azetidin-3-yl)methoxy]-2-methyl-pyrazol-3-yl]pyrazolo[1,5-a]pyridin-2-yl]cyclopropanecarboxamide;
N-[5-[4-[(3-methoxy-1-methyl-azetidin-3-yl)methoxy]-2-methyl-pyrazol-3-yl]pyrazolo[1,5-a]pyridin-2-yl]cyclopropanecarboxamide;
N-[5-[2-methyl-4-(4-piperidyloxy)pyrazol-3-yl]pyrazolo[1,5-a]pyridin-2-yl]cyclopropanecarboxamide;
N-[5-[4-[(3-ethyl-1-methyl-azetidin-3-yl)methoxy]-2-methyl-pyrazol-3-yl]pyrazolo[1,5-a]pyridin-2-yl]cyclopropanecarboxamide;
(rac-cis)-N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide;
N-[5-[2-methyl-4-[(1-methyl-3-phenyl-azetidin-3-yl)methoxy]pyrazol-3-yl]pyrazolo[1,5-a]pyridin-2-yl]cyclopropanecarboxamide;
(3*R,4*S)—N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide;
N-[5-[2-methyl-4-[[(2S)-pyrrolidin-2-yl]methoxy]pyrazol-3-yl]pyrazolo[1,5-a]pyridin-2-yl]cyclopropanecarboxamide;
(3*S,4*S)—N-[5-[2-methyl-4-(4-methylpyrrolidin-3-yl)oxy-pyrazol-3-yl]pyrazolo[1,5-a]pyridin-2-yl]cyclopropanecarboxamide;
(3*R,4*R)—N-[5-[4-(4-hydroxypyrrolidin-3-yl)oxy-2-methyl-pyrazol-3-yl]pyrazolo[1,5-a]pyridin-2-yl]cyclopropanecarboxamide;
N-[5-[4-[(3R,4S)-4-fluoro-1-methyl-pyrrolidin-3-yl]oxy-2-methyl-pyrazol-3-yl]pyrazolo[1,5-a]pyridin-2-yl]cyclopropanecarboxamide;
N-[5-[4-[[(3S,4S)-4-hydroxypyrrolidin-3-yl]methoxy]-2-methyl-pyrazol-3-yl]pyrazolo[1,5-a]pyridin-2-yl]cyclopropanecarboxamide;
N-[5-[4-[[(3S,4S)-4-hydroxy-1-methyl-pyrrolidin-3-yl]methoxy]-2-methyl-pyrazol-3-yl]pyrazolo[1,5-a]pyridin-2-yl]cyclopropanecarboxamide;
(3*R, 4*R)—N—[5-[4-(1,4-dimethylpyrrolidin-3-yl)oxy-2-methyl-pyrazol-3-yl]pyrazolo[1,5-a]pyridin-2-yl]cyclopropanecarboxamide;
N-[5-[2-methyl-4-[[(2S)-1-methylpyrrolidin-2-yl]methoxy]pyrazol-3-yl]pyrazolo[1,5-a]pyridin-2-yl]cyclopropanecarboxamide;
N-[5-[4-[[(2S)-1-ethylpyrrolidin-2-yl]methoxy]-2-methyl-pyrazol-3-yl]pyrazolo[1,5-a]pyridin-2-yl]cyclopropanecarboxamide;
(rac-trans)-N-[5-[4-(4-methoxypyrrolidin-3-yl)oxy-2-methyl-pyrazol-3-yl]pyrazolo[1,5-a]pyridin-2-yl]cyclopropanecarboxamide;
N-[5-[4-[[(3S,4S)-4-methoxypyrrolidin-3-yl]methoxy]-2-methyl-pyrazol-3-yl]pyrazolo[1,5-a]pyridin-2-yl]cyclopropanecarboxamide;
N-[5-[4-[[(3S,4S)-4-methoxy-1-methyl-pyrrolidin-3-yl]methoxy]-2-methyl-pyrazol-3-yl]pyrazolo[1,5-a]pyridin-2-yl]cyclopropanecarboxamide;
N-[5-[4-[[(3R,4R)-4-hydroxy-1-methyl-pyrrolidin-3-yl]methoxy]-2-methyl-pyrazol-3-yl]pyrazolo[1,5-a]pyridin-2-yl]cyclopropanecarboxamide;
N-[5-[4-[[(2R)-1-(cyanomethyl)azetidin-2-yl]methoxy]-2-methyl-pyrazol-3-yl]pyrazolo[1,5-a]pyridin-2-yl]cyclopropanecarboxamide;
N-[5-[4-[(2R)-2-amino-3-hydroxy-3-methyl-butoxy]-2-methyl-pyrazol-3-yl]pyrazolo[1,5-a]pyridin-2-yl]cyclopropanecarboxamide;
N-[5-[4-[[(2R)-1-ethylpyrrolidin-2-yl]methoxy]-2-methyl-pyrazol-3-yl]pyrazolo[1,5-a]pyridin-2-yl]cyclopropanecarboxamide;
N-[5-[4-[[(2R)-1-(2-methoxyethyl)pyrrolidin-2-yl]methoxy]-2-methyl-pyrazol-3-yl]pyrazolo[1,5-a]pyridin-2-yl]cyclopropanecarboxamide;
N-[5-[4-[[(2R)-1-benzylazetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-[(1-methylpyrazol-3-yl)methyl]azetidin-2-yl]methoxy]pyrazol-3-yl]pyrazolo[1,5-a]pyridin-2-yl]cyclopropanecarboxamide;
N-[5-[2-methyl-4-[[(2R)-1-[2-(trifluoromethoxy)ethyl]azetidin-2-yl]methoxy]pyrazol-3-yl]pyrazolo[1,5-a]pyridin-2-yl]cyclopropanecarboxamide;
N-[5-[2-methyl-4-[[(1S,4R,5R)-3-methyl-3-azabicyclo[3.1.0]hexan-4-yl]methoxy]pyrazol-3-yl]pyrazolo[1,5-a]pyridin-2-yl]cyclopropanecarboxamide;
N-[5-[2-methyl-4-[[(2R)-1-(oxetan-3-ylmethyl)azetidin-2-yl]methoxy]pyrazol-3-yl]pyrazolo[1,5-a]pyridin-2-yl]cyclopropanecarboxamide;
N-[5-[2-methyl-4-[[1-(methylamino)cyclopropyl]methoxy]pyrazol-3-yl]pyrazolo[1,5-a]pyridin-2-yl]cyclopropanecarboxamide;
N-[5-[4-[[(2R)-1-(2-methoxy-2-methyl-propyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-(2-pyridyl)azetidin-2-yl]methoxy]pyrazol-3-yl]pyrazolo[1,5-a]pyridin-2-yl]cyclopropanecarboxamide;
N-[5-[4-[[(2R,3S)-1,3-dimethylazetidin-2-yl]methoxy]-2-methyl-pyrazol-3-yl]pyrazolo[1,5-a]pyridin-2-yl]cyclopropanecarboxamide;
3-[[5-[2-[(2,6-dimethylpyrimidin-4-yl)amino]pyrazolo[1,5-a]pyridin-5-yl]-1-methyl-pyrazol-4-yl]oxymethyl]cyclobutanol;
(*S)-3-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol;
(*S)—N-(2,6-dimethylpyrimidin-4-yl)-5-[2-methyl-4-[(3-methyltetrahydrofuran-3-yl)methoxy]pyrazol-3-yl]pyrazolo[1,5-a]pyridin-2-amine;
(1*R, 2*S, 4*S)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine;
N-(2,6-dimethylpyrimidin-4-yl)-5-[2-methyl-4-[(2R,3S)-2-methylpyrrolidin-3-yl]oxy-pyrazol-3-yl]pyrazolo[1,5-a]pyridin-2-amine;
(*S)—N-(2,6-dimethylpyrimidin-4-yl)-5-[2-methyl-4-(8-oxaspiro[4.4]nonan-7-ylmethoxy)pyrazol-3-yl]pyrazolo[1,5-a]pyridin-2-amine;
(*R)-1-cyclobutyl-2-[5-[2-[(2,6-dimethylpyrimidin-4-yl)amino]pyrazolo[1,5-a]pyridin-5-yl]-1-methyl-pyrazol-4-yl]oxy-ethanol;
(3*S,4*S)—N-(2,6-dimethylpyrimidin-4-yl)-5-[2-methyl-4-(4-methyltetrahydrofuran-3-yl)oxy-pyrazol-3-yl]pyrazolo[1,5-a]pyridin-2-amine;
N-(2,6-dimethylpyrimidin-4-yl)-5-[2-methyl-4-[[(2R,4S)-4-methylpyrrolidin-2-yl]methoxy]pyrazol-3-yl]pyrazolo[1,5-a]pyridin-2-amine;
(*S)—N-(2,6-dimethylpyrimidin-4-yl)-5-[2-methyl-4-[(2-methyltetrahydrofuran-2-yl)methoxy]pyrazol-3-yl]pyrazolo[1,5-a]pyridin-2-amine;
(1*S,2*S)-2-[[5-[2-[(2,6-dimethylpyrimidin-4-yl)amino]pyrazolo[1,5-a]pyridin-5-yl]-1-methyl-pyrazol-4-yl]oxymethyl]cyclobutanol;
5-[2-methyl-4-[(3S)-1-methylpyrrolidin-3-yl]oxy-pyrazol-3-yl]-N-pyridazin-3-yl-pyrazolo[1,5-a]pyridin-2-amine;
5-[2-methyl-4-[(3S)-1-methylpyrrolidin-3-yl]oxy-pyrazol-3-yl]-N-pyrazin-2-yl-pyrazolo[1,5-a]pyridin-2-amine;
5-[2-methyl-4-[(3S)-1-methylpyrrolidin-3-yl]oxy-pyrazol-3-yl]-N-(6-methyl-3-pyridyl)pyrazolo[1,5-a]pyridin-2-amine;
5-[2-methyl-4-[(3S)-1-methylpyrrolidin-3-yl]oxy-pyrazol-3-yl]-N-(2-pyridyl)pyrazolo[1,5-a]pyridin-2-amine;
5-[2-methyl-4-[(3S)-1-methylpyrrolidin-3-yl]oxy-pyrazol-3-yl]-N-[5-(trifluoromethyl)pyrazin-2-yl]pyrazolo[1,5-a]pyridin-2-amine;
5-[2-methyl-4-[(3S)-1-methylpyrrolidin-3-yl]oxy-pyrazol-3-yl]-N-(5-methylpyrazin-2-yl)pyrazolo[1,5-a]pyridin-2-amine;
5-[2-methyl-4-[(3S)-1-methylpyrrolidin-3-yl]oxy-pyrazol-3-yl]-N-pyrimidin-4-yl-pyrazolo[1,5-a]pyridin-2-amine;
5-[2-methyl-4-[(3S)-1-methylpyrrolidin-3-yl]oxy-pyrazol-3-yl]-N-(2-methylpyrimidin-4-yl)pyrazolo[1,5-a]pyridin-2-amine;
5-[2-methyl-4-[(3S)-1-methylpyrrolidin-3-yl]oxy-pyrazol-3-yl]-N-[5-(trifluoromethyl)pyridazin-3-yl]pyrazolo[1,5-a]pyridin-2-amine;
5-[2-methyl-4-[(3S)-1-methylpyrrolidin-3-yl]oxy-pyrazol-3-yl]-N-[2-(trifluoromethyl)pyrimidin-4-yl]pyrazolo[1,5-a]pyridin-2-amine;
5-[2-methyl-4-[(3S)-1-methylpyrrolidin-3-yl]oxy-pyrazol-3-yl]-N-[6-(trifluoromethyl)pyrimidin-4-yl]pyrazolo[1,5-a]pyridin-2-amine;
1-methyl-N-[5-[2-methyl-4-[(3S)-1-methylpyrrolidin-3-yl]oxy-pyrazol-3-yl]pyrazolo[1,5-a]pyridin-2-yl]pyrrolo[2,3-b]pyridin-5-amine;
N-[6-(methoxymethyl)pyrimidin-4-yl]-5-[2-methyl-4-[(3S)-1-methylpyrrolidin-3-yl]oxy-pyrazol-3-yl]pyrazolo[1,5-a]pyridin-2-amine;
N-(6-cyclopropylpyridazin-3-yl)-5-[2-methyl-4-[(3S)-1-methylpyrrolidin-3-yl]oxy-pyrazol-3-yl]pyrazolo[1,5-a]pyridin-2-amine;
N-[5-[2-methyl-4-(4-piperidyloxymethyl)pyrazol-3-yl]pyrazolo[1,5-a]pyridin-2-yl]cyclopropanecarboxamide;
N-[5-[2-methyl-4-[[(3S)-1-methylpyrrolidin-3-yl]oxymethyl]pyrazol-3-yl]pyrazolo[1,5-a]pyridin-2-yl]cyclopropanecarboxamide;
(*S)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[4-[[(3R,4R)-4-fluoropyrrolidin-3-yl]oxymethyl]-2-methyl-pyrazol-3-yl]pyrazolo[1,5-a]pyridin-2-yl]cyclopropanecarboxamide;
N-[5-[4-[[(3R,4R)-4-fluoro-1-methyl-pyrrolidin-3-yl]oxymethyl]-2-methyl-pyrazol-3-yl]pyrazolo[1,5-a]pyridin-2-yl]cyclopropanecarboxamide;
N-[5-[4-[(3-ethyl-1-methyl-azetidin-3-yl)oxymethyl]-2-methyl-pyrazol-3-yl]pyrazolo[1,5-a]pyridin-2-yl]cyclopropanecarboxamide;
(rac-trans)-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
N-[5-[2-(difluoromethyl)-4-[(3S)-1-methylpyrrolidin-3-yl]oxy-pyrazol-3-yl]pyrazolo[1,5-a]pyridin-2-yl]cyclopropanecarboxamide;
N-[6-[2-methyl-4-[(3S)-pyrrolidin-3-yl]oxy-pyrazol-3-yl]imidazo[1,2-a]pyridin-2-yl]cyclopropanecarboxamide;
N-[6-[2-methyl-4-[(3S)-1-methylpyrrolidin-3-yl]oxy-pyrazol-3-yl]imidazo[1,2-a]pyridin-2-yl]cyclopropanecarboxamide;
cis-N-[5-[2-methyl-4-[(1-methylazetidin-2-yl)methoxy]pyrazol-3-yl]pyrazolo[1,5-a]pyridin-2-yl]-2-phenyl-cyclopropanecarboxamide;
(1*R,2*S)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide;
cis-2-cyano-N-[5-[2-methyl-4-[(1-methylazetidin-2-yl)methoxy]pyrazol-3-yl]pyrazolo[1,5-a]pyridin-2-yl]cyclopropanecarboxamide;
N-(2,6-dimethylpyrimidin-4-yl)-6-[4-[[(2R)-1-ethylazetidin-2-yl]methoxy]-2-methyl-pyrazol-3-yl]imidazo[1,2-a]pyridin-2-amine;
5-[2-(difluoromethyl)-4-[[(2R)-1-ethylazetidin-2-yl]methoxy]pyrazol-3-yl]-N-(2-methylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(2,6-dimethylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyridazin-3-yl)pyrazolo[1,5-a]pyridin-2-amine;
5-[2-(difluoromethyl)-4-[[(2R)-1-ethylazetidin-2-yl]methoxy]pyrazol-3-yl]-N-(2-methoxypyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyrazin-2-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methoxy-5-methyl-pyridazin-3-yl)pyrazolo[1,5-a]pyridin-2-amine;
6-[[5-[2-(difluoromethyl)-4-[[(2R)-1-ethylazetidin-2-yl]methoxy]pyrazol-3-yl]pyrazolo[1,5-a]pyridin-2-yl]amino]-2,4-dimethyl-pyridazin-3-one;
N-(2,6-dimethylpyrimidin-4-yl)-6-[2-methyl-4-[[(2R)-1-methylazetidin-2-yl]methoxy]pyrazol-3-yl]imidazo[1,2-a]pyridin-2-amine;
N-[5-(5-methylisoxazol-4-yl)pyrazolo [1,5-a]pyridin-2-yl]cyclopropanecarboxamide;
N-[5-[2-methyl-4-[[(2S)-4-methylmorpholin-2-yl]methoxy]pyrazol-3-yl]pyrazolo[1,5-a]pyridin-2-yl]cyclopropanecarboxamide;
N-[5-[2-methyl-4-[(3R)-1-methylpyrrolidin-3-yl]oxy-pyrazol-3-yl]pyrazolo[1,5-a]pyridin-2-yl]cyclopropanecarboxamide;
N-[5-[2-(cyclopropylmethyl)-4-[[(3R)-1-methylpyrrolidin-3-yl]methoxy]pyrazol-3-yl]pyrazolo[1,5-a]pyridin-2-yl]cyclopropanecarboxamide;
N-[5-[4-[[(3R)-1-methylpyrrolidin-3-yl]methoxy]-2-propyl-pyrazol-3-yl]pyrazolo[1,5-a]pyridin-2-yl]cyclopropanecarboxamide;
N-[5-[2-benzyl-4-[[(3R)-1-methylpyrrolidin-3-yl]methoxy]pyrazol-3-yl]pyrazolo[1,5-a]pyridin-2-yl]cyclopropanecarboxamide;
N-[5-[2-methyl-4-[(1-methyl-4-piperidyl)oxy]pyrazol-3-yl]pyrazolo[1,5-a]pyridin-2-yl]cyclopropanecarboxamide;
N-[5-[5-methyl-3-[(1-methyl-4-piperidyl)oxy]isoxazol-4-yl]pyrazolo[1,5-a]pyridin-2-yl]cyclopropanecarboxamide;
N-[5-[5-methyl-3-(1-methylazetidin-3-yl)oxy-isoxazol-4-yl]pyrazolo[1,5-a]pyridin-2-yl]cyclopropanecarboxamide;
N-[5-[5-methyl-3-[(1-methylazetidin-3-yl)oxymethyl]isoxazol-4-yl]pyrazolo[1,5-a]pyridin-2-yl]cyclopropanecarboxamide;
N-[5-[5-methyl-3-[[(3R)-morpholin-3-yl]methoxy]isoxazol-4-yl]pyrazolo[1,5-a]pyridin-2-yl]cyclopropanecarboxamide;
N-[5-[5-methyl-3-[[(3S)-morpholin-3-yl]methoxy]isoxazol-4-yl]pyrazolo[1,5-a]pyridin-2-yl]cyclopropanecarboxamide;
N-[5-[5-methyl-3-[[(2S)-1-methylazetidin-2-yl]methoxy]isoxazol-4-yl]pyrazolo[1,5-a]pyridin-2-yl]cyclopropanecarboxamide;
N-[5-[4-[(3S,4R)-4-fluoro-1-methyl-pyrrolidin-3-yl]oxy-2-methyl-pyrazol-3-yl]pyrazolo[1,5-a]pyridin-2-yl]cyclopropanecarboxamide;
N-[5-[5-methyl-3-[[(2S)-1-methylpyrrolidin-2-yl]methoxy]isoxazol-4-yl]pyrazolo[1,5-a]pyridin-2-yl]cyclopropanecarboxamide;
N-[5-[5-methyl-3-[[(2S)-1-methyl-2-piperidyl]methoxy]isoxazol-4-yl]pyrazolo[1,5-a]pyridin-2-yl]cyclopropanecarboxamide;
N-[5-[5-methyl-3-[[(2R)-1-methyl-2-piperidyl]methoxy]isoxazol-4-yl]pyrazolo[1,5-a]pyridin-2-yl]cyclopropanecarboxamide;
5-[2-methyl-4-[[(2R)-1-methylazetidin-2-yl]methoxy]pyrazol-3-yl]pyrazolo[1,5-a]pyridin-2-amine;
N-[5-[4-[(3S,4R)-4-fluoropyrrolidin-3-yl]oxy-2-methyl-pyrazol-3-yl]pyrazolo[1,5-a]pyridin-2-yl]cyclopropanecarboxamide;
N-[5-[2-methyl-4-[[(2S)-1-methyl-2-piperidyl]methoxy]pyrazol-3-yl]pyrazolo[1,5-a]pyridin-2-yl]cyclopropanecarboxamide;
N-[5-[4-[(1-aminocyclopropyl)methoxy]-2-methyl-pyrazol-3-yl]pyrazolo[1,5-a]pyridin-2-yl]cyclopropanecarboxamide; and
N-[5-[2-methyl-4-[[(2R)-1-methyl-2-piperidyl]methoxy]pyrazol-3-yl]pyrazolo[1,5-a]pyridin-2-yl]cyclopropanecarboxamide;

or a tautomer, stereoisomer pharmaceutically acceptable salt, or solvate thereof.

### Methods of Preparation

Compounds described herein can be prepared by any number of processes as described generally below and more specifically illustrated by the exemplary compounds which follow in the Examples section herein. The compounds provided herein as prepared in the processes described below can be synthesized in the form of mixtures of stereoisomers (e.g., enantiomers, diastereomers), including racemic mixtures of enantiomers, that can be separated from one another using art-known resolution procedures, for instance including liquid chromatography using a chiral stationary phase. Additionally or alternatively, stereochemically pure isomeric forms of the compounds described herein can be derived from the corresponding stereochemically pure isomeric forms of the appropriate starting materials, intermediates, or reagents. For example, if a specific stereoisomer is desired, the compound can be synthesized by stereospecific methods of preparation, which typically employ stereochemically pure starting materials or intermediate compounds.

Pharmaceutically acceptable salts of compounds of the application can be synthesized from the parent compound containing an acidic or basic moiety by conventional chemical methods. Generally, such salts can be prepared by reacting the free acid or base forms of these compounds with a stoichiometric amount of the appropriate acid or base in water or in an organic solvent, or in a mixture of the two. Examples of suitable organic solvents include, but are not limited to, ether, ethyl acetate (EtOAc), ethanol, isopropanol, or acetonitrile.

By way of illustration, but not as a limitation, compounds of formula (II) described herein can be prepared according to the following general preparation procedures shown in Schemes 1 and 2, and compounds of formula (III) as described herein can be prepared according to the following general preparation procedures shown in Schemes 3 and 4. One of ordinary skill in the art will recognize that, to obtain various compounds of formula (II) and compounds of formula (III) as described herein, starting materials can be suitably selected so that the ultimately desired substituent groups will be carried through (i.e., be stable over the course of the synthesis) the reaction scheme with or without protection as appropriate to yield the desired product. Alternatively, it may be necessary or desirable to employ, in place of the ultimately desired substituent, a suitable group that may be carried through (i.e., be stable over the course of the synthesis) the reaction scheme and replaced as appropriate with the desired substituent.

Unless otherwise specified, the variables in Schemes 1-4 are as defined above in reference to the various embodiments of compounds of formula (II) and compounds of formula (III). The following general features concerning reaction temperatures, protecting groups (PGs), leaving groups (LGs), and substituents R₁, R₂, R₃, R₄, R₅ etc. refer, as applicable, to Schemes 1-4.

If no temperature or temperature range is stated, it is to be understood that the reaction is to be conducted at room temperature.

The term PG in the following Schemes 1-4 represents a protecting group. Examples of protecting groups suitable for use include, but are not limited to, acetyl (Ac), t-butylcarbamate (Boc), and t-butyldimethylsilyl (TBS).

The term LG in the following Schemes 1-4 represents a leaving group. Examples of leaving groups suitable for use include, but are not limited to, halo (e.g., I, Cl, Br, F), mesylate (-OMs), triflate (-OTf), tosylate (-OTs), boronic acid, and boronate ester.

When isomerically pure samples are desired, isomeric mixtures of compounds synthesized according to Scheme 1-4 can be separated by chiral supercritical fluid chromatography (SFC) or high performance liquid chromatography (HPLC).

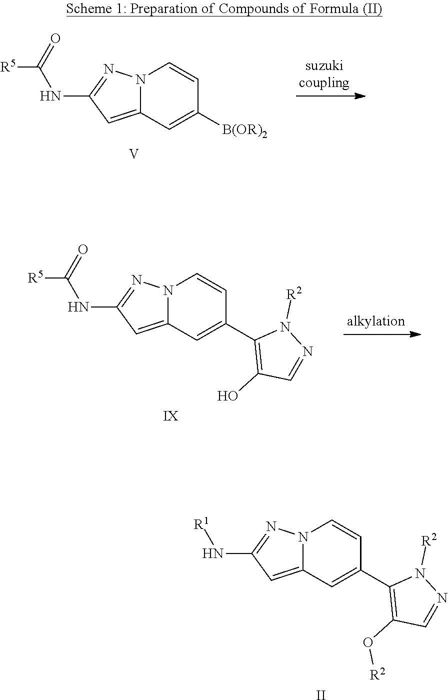

As shown in Scheme 1, a compound of formula (IX) is prepared from a compound of formula (V) by a metal-mediated cross coupling reaction with a suitably substituted commercially available or synthetically accessible pyrazole, substituted with Br, Cl, OTf, or I, in the presence of a palladium catalyst such as PdCl₂(dtbpf), Pd(PPh₃)₄, PdCl₂(dppf), Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, water, or a mixture thereof; at a temperature ranging from 60 to 180° C., employing microwave or conventional heating; for a period of about 30 min to 16 h, to provide a compound of Formula (IX). A compound of formula (IX) is reacted with a suitable electrophile such as R²-LG with or without a suitable base such as TEA, Cs₂CO₃, K₂CO₃, or sodium hydride in a suitable solvent such as DMF, THF, DMA, DCM, and the like, for a period of 1-16 h, to provide a compound of formula (II), where R² is defined as above. A compound of formula (IX) can also react with a suitable alcohol with or without a suitable activating agent and suitable trialkyl- or triaryl-phosphine in a suitable solvent such as DMF, toluene, DCM, THF, DMA, DCE, and a mixture thereof; at temperature between 25 to 180° C., employing microwave or conventional heating; for a period of 1-16 h to provide a compound of formula (II).

Conversion of compounds of Formula (II), wherein R² contains a protected or unprotected amine, to a compound of Formula (II), the amine may be substituted with methyl or alkyl with a suitable aldehyde or ketone or similar equivalents such as paraformaldehyde, acetaldehyde, a reducing agent such as NaBH(OAc)₃, NaBH₄, NaCNBH₃ and the like, in a solvent such as THF, DCM, MeOH and the like, for a period of 12-20 h.

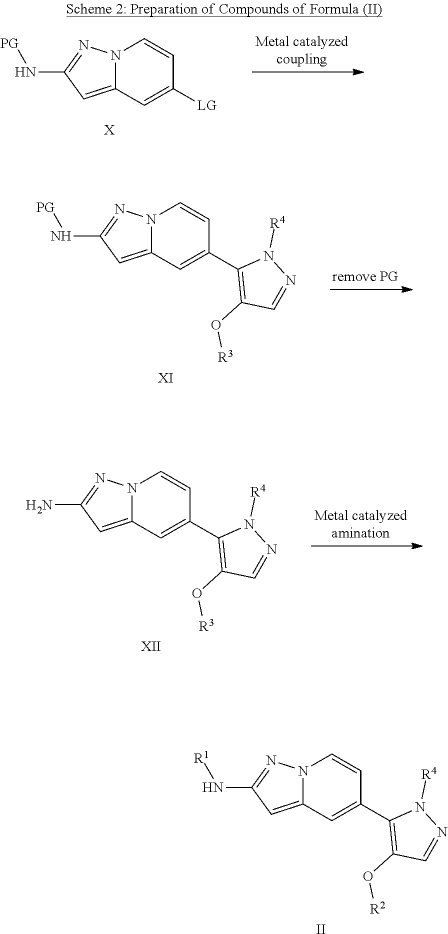

As shown in Scheme 2, a compound of formula (XI) is prepared from a compound of formula (X) by a metal-mediated cross coupling reaction, where PG is Boc, CBz, and the like; LG is Br, Cl, F, I, boronic acid or boronate esters with suitable substitution. For example, a compound of formula (X), is reacted with a suitably substituted commercially available or synthetically accessible aryl-Br, and the like, in the presence of a palladium catalyst such as PdCl₂(dtbpf), Pd(PPh₃)₄, PdCl₂(dppf), Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, dioxane, THF, water, or a mixture thereof; at a temperature ranging from 60 to 180° C., employing microwave or conventional heating; for a period of about 30 min to 16 h, to provide a compound of Formula (XI). A compound of formula (XI) is reacted with suitable reagents to remove the protecting group (PG) to provide a compound of formula (XII). A compound of formula (XII) is then reacted under metal-mediated cross coupling to form a compound of formula (II). For example, a compound of formula (XII), is reacted with a commercially available or synthetically accessible suitably substituted aryl-halide or heteroaryl-halide; in the presence of a palladium catalyst such as tert-butyl Brettphos G2, and the like; a suitable base such a Na₂CO₃, potassium phosphate, Cs₂CO₃, K₂CO₃, and the like; in a solvent such as 1,4-dioxane; employing conventional or microwave heating; at temperatures ranging from room temperature to 100° C.; for a period of 1 h to 18 h, to give a compound of formula (II). Conversion of compounds of formula (II), wherein R² contains a protected or unprotected amine, to a compound of formula (II), wherein amine is substituted with methyl or alkyl, is achieved with deprotection under suitable conditions if necessary and then reacted in a reductive amination reaction with a suitable aldehyde or ketone or similar equivalents such as paraformaldehyde, acetaldehyde and the like, a reducing agent such as NaBH(OAc)₃, NaBH₄, NaCNBH₃ and the like, in a solvent such as THF, DCM, MeOH and the like, for a period of 6-24 h.

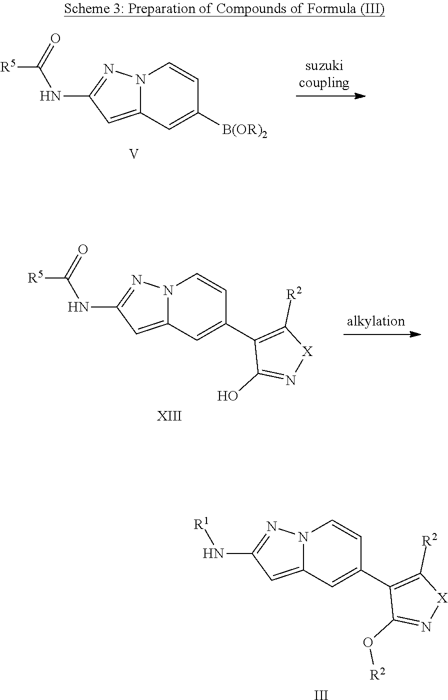

As shown in Scheme 3, a compound of formula (XIII) is prepared from a compound of formula (V) by a metal-mediated cross coupling reaction with a suitably substituted commercially available or synthetically accessible pyrazole, substituted with Br, Cl, OTf, or I, in the presence of a palladium catalyst such as PdCl₂(dtbpf), Pd(PPh₃)₄, PdCl₂(dppf), Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, water, or a mixture thereof; at a temperature ranging from 60 to 180° C., employing microwave or conventional heating; for a period of about 30 min to 16 h, to provide a compound of formula (XIII). A compound of formula (XIII) is reacted with a suitable electrophile such as R₂-LG with or without a suitable base such as TEA, Cs₂CO₃, K₂CO₃, or sodium hydride in a suitable solvent such as DMF, THF, DMA, DCM, and the like, for a period of 1-16 h, to provide a compound of formula (III), where R² is defined as above. A compound of formula (XIII) can also react with a suitable alcohol with or without a suitable activating agent and suitable trialkyl- or triaryl-phosphine in a suitable solvent such as DMF, toluene, DCM, THF, DMA, DCE, and a mixture thereof; at a temperature between 25 to 180° C., employing microwave or conventional heating; for a period of 1-18 h to provide a compound of formula (III).

Conversion of compounds of Formula (III), wherein R² contains a protected or unprotected amine, to a compound of Formula (III), the amine may be substituted with methyl or alkyl with a suitable aldehyde or ketone or similar equivalents such as paraformaldehyde, acetaldehyde, a reducing agent such as NaBH(OAc)₃, NaBH₄, NaCNBH₃ and the like, in a solvent such as THF, DCM, MeOH and the like, for a period of 12-20 h.

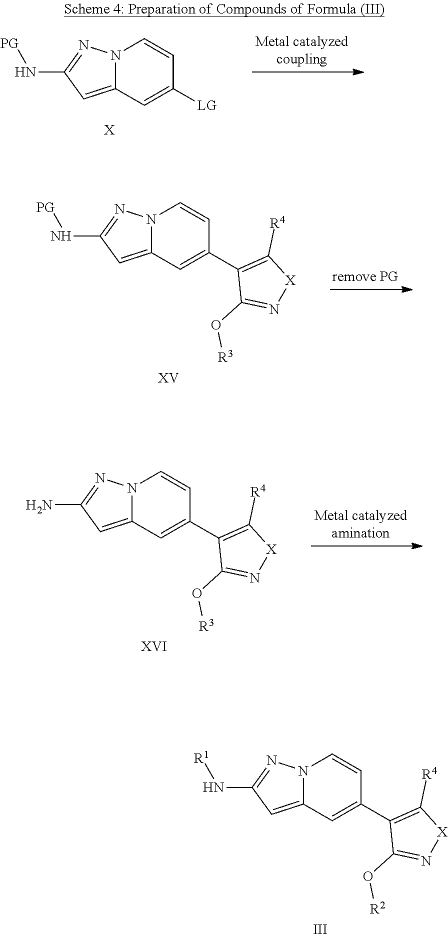

As shown in Scheme 4, a compound of formula (XV) is prepared from a compound of formula (X) by a metal-mediated cross coupling reaction, where PG is Boc, CBz, and the like; LG is Br, Cl, F, I, boronic acid or boronate esters with suitable substitution. For example, a compound of formula (X), is reacted with a suitably substituted commercially available or synthetically accessible aryl-Br, and the like, in the presence of a palladium catalyst such as PdCl₂(dtbpf), Pd(PPh₃)₄, PdCl₂(dppf), Pd(PPh₃)₂Cl₂, Pd(OAc)₂, and the like; with or without the addition of a ligand such as DPPF; a base such as K₃PO₄, K₂CO₃, aq. Na₂CO₃, Na₂CO₃, Cs₂CO₃, and the like; in a suitable solvent such as 1,2-dimethoxyethane, 1,4-dioxane, DMF, dioxane, THF, water, or a mixture thereof; at a temperature ranging from 60 to 180° C., employing microwave or conventional heating; for a period of about 30 min to 16 h, to provide a compound of Formula (XV). A compound of formula (XV) is reacted with suitable reagents to remove the protecting group (PG) to provide a compound of formula (XVI). A compound of formula (XVI) is then reacted under metal-mediated cross coupling to form a compound of formula (III). For example, a compound of formula (XVI), is reacted with a commercially available or synthetically accessible suitably substituted aryl-halide or heteroaryl-halide; in the presence of a palladium catalyst such as tert-butyl Brettphos G2, and the like; a suitable base such a Na₂CO₃, potassium phosphate, Cs₂CO₃, K₂CO₃, and the like; in a solvent such as 1,4-dioxane; employing conventional or microwave heating; at temperatures ranging from room temperature to 100° C.; for a period of 1 h to 18 h, to give a compound of formula (III). Conversion of compounds of formula (III), wherein R² contains a protected or unprotected amine, to a compound of formula (III), wherein amine is substituted with methyl or alkyl, is achieved with deprotection under suitable conditions if necessary and then reacted in a reductive amination reaction with a suitable aldehyde or ketone or similar equivalents such as paraformaldehyde, acetaldehyde and the like, a reducing agent such as NaBH(OAc)₃, NaBH₄, NaCNBH₃ and the like, in a solvent such as THF, DCM, MeOH and the like, for a period of 6-24 h.

### Compositions

In another aspect, provided is a pharmaceutical composition comprising a compound of formula (II) or a compound of formula (III) or a tautomer, stereoisomer, pharmaceutically acceptable salt or solvate thereof, as described herein.

Compositions can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions can also be formulated for other routes of administration including transmucosal, ocular, rectal, long acting implantation, sublingual administration, under the tongue, from oral mucosa bypassing the portal circulation, inhalation, or intranasal.

In yet another aspect, provided is a method of preparing a pharmaceutical composition comprising combining a compound of formula (II) or a compound of formula (III), or a tautomer, stereoisomer, pharmaceutically acceptable salt or solvate thereof, with at least one pharmaceutically acceptable carrier. Pharmaceutical compositions can be prepared by any method known in the art in view of the present disclosure, and one of ordinary skill in the art will be familiar with such techniques used to prepare pharmaceutical compositions. For example, a pharmaceutical composition according to the application can be prepared by mixing a compound of formula (II) or a compound of formula (III), with one or more pharmaceutically acceptable carriers according to conventional pharmaceutical compounding techniques, including but not limited to, conventional admixing, dissolving, granulating, emulsifying, encapsulating, entrapping or lyophilizing processes.

### Methods of Use

In another general aspect, provided are methods of inhibiting a salt inducible kinase (SIK) and methods of treating a disease related to SIKs, including autoimmune disorders and proliferative disorders, using the compounds and compositions, e.g., pharmaceutical compositions, described herein.

As used herein, “an effective amount” means an amount of a composition or compound that elicits a biological or medicinal response in a tissue system or subject that is being sought by a researcher, veterinarian, medical doctor or other conditions, which can include alleviation of the symptoms of the disease, disorder, or condition being treated. An effective amount can vary depending upon a variety of factors, such as the physical condition of the subject, age, weight, health, etc.; and the particular disease, disorder, or condition to be treated. An effective amount can readily be determined by one of ordinary skill in the art in view of the present disclosure.

According to particular embodiments, an effective amount refers to the amount of a composition or compound described herein which is sufficient to inhibit a salt inducible kinase (SIK). In another particular embodiment, an effective amount refers to the amount of a composition or compound described herein which is sufficient to treat a disease, disorder, or condition mediated by a SIK.

In an embodiment, provided is a method of inhibiting a salt inducible kinase (SIK) in a subject in need thereof, comprising administering to the subject a compound or composition described herein, e.g., administering an effective amount of a compound or composition described herein.

In some embodiments, the salt inducible kinase is SIK1.

In some embodiments, the salt inducible kinase is SIK2.

In some embodiments, the salt inducible kinase is SIK3.

In some embodiments, the salt inducible kinase is SIK1 and SIK2.

In some embodiments, the salt inducible kinase is SIK1 and SIK3.

In some embodiments, the salt inducible kinase is SIK2 and SIK3.

In some embodiments, the salt inducible kinase is SIK1, SIK2, and SIK3.

In an embodiment, provided is a method of treating a disease, disorder, or condition mediated by a SIK in a subject in need thereof, comprising administering to the subject a compound or composition described herein, e.g., administering an effective amount of a compound or composition described herein.

In some embodiments, a disease, disorder, or condition is mediated by SIK1.

In some embodiments, a disease, disorder, or condition is mediated by SIK2.

In some embodiments, a disease, disorder, or condition is mediated by SIK3.

In some embodiments, a disease, disorder, or condition is mediated by SIK1 and SIK2.

In some embodiments, a disease, disorder, or condition is mediated by SIK1 and SIK3.

In some embodiments, a disease, disorder, or condition is mediated by SIK2 and SIK3.

In some embodiments, a disease, disorder, or condition is mediated by SIK1, SIK2, and SIK3.

In some embodiments, a disease, disorder, or condition mediated by a SIK is an autoimmune disorder.

In some embodiments, a disease, disorder, or condition mediated by a SIK is a proliferative disorder.

In other general aspects, provided are compounds and compositions described herein for use in methods of inhibiting a salt inducible kinase (SIK) and/or methods of treating a disease, disorder, or condition mediated by a SIK. In yet other general aspects, provided are uses of the compounds and compositions described herein in the manufacture of a medicament for inhibiting a salt inducible kinase (SIK) and/or for treating a disease, disorder, or condition mediated by a SIK.

### EXAMPLES

The following examples of the application are to further illustrate the nature of the application. It should be understood that the following examples do not limit the application and the scope of the application is to be determined by the appended claims.

### Synthesis Examples

### Intermediate 1: 5-bromopyrazolo[1,5-a]pyridin-2-amine

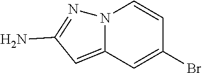 [SMILES: Nc1cc2cc(Br)ccn2n1]

Step A: 2-(4-bromopyridin-2-yl)acetonitrile. To a solution of n-BuLi (1.9 moles) in THE (800 mL, 8 mL/g) cooled to −78° C. was charged acetonitrile (2.1 moles) while maintaining an internal temperature of −78 C±5° C. The resulting mixture was stirred at −78° C. for 45 minutes and then was charged with a solution of 4-bromo-2-fluoropyridine (100 g, 0.57 moles) in THE (200 mL, 2 mL/g) while maintaining an internal temperature of −78±5° C. The reaction mixture was warmed to −30° C. and stirred for 2 hours. Once HPLC analysis indicated complete consumption of all the starting material, the reaction was quenched by the slow addition of saturated aqueous ammonium chloride (100 mL, 1 mL/g) and then warmed to 23° C. The mixture was concentrated and then diluted with water (1000 mL, 10 mL/g) followed by EtOAc (1000 mL, 10 mL/g). The layers were separated, and the organic phase was washed with brine (1000 mL, 10 mL/g) and dried with Na₂SO₄. The organic phase was filtered and concentrated in vacuo to give a black oil which was then purified by silica gel chromatography (ether:EtOAc=15:1 to 5:1) to give the title compound as a yellow solid (87 g, 0.44 moles, 77%).

Step B: 1-amino-4-bromo-2-(cyanomethyl)pyridin-1-ium 2,4,6-trimethylbenzenesulfonate. To a solution of O-(mesitylsulfonyl)hydroxylamine (1.8 kg, 8.2 moles) in DCM (9.0 L, 10 L/kg) which had been cooled to 5±5° C. was charged 2-(4-bromopyridin-2-yl)acetonitrile (0.90 kg, 4.6 moles) while maintaining an internal temperature of 5±5° C. The reaction mixture was slowly warmed to 23° C. and stirred for 19 hours. The resulting slurry was then filtered and washed with DCM (1.8 L, 2 L/kg) and dried to give the title compound as an off-white solid (2.15 kg) which was stored in the freezer and used without further purification in the following step.

Step C: 5-bromopyrazolo[1,5-a]pyridin-2-amine. To a solution of 1-amino-4-bromo-2-(cyanomethyl)pyridin-1-ium 2,4,6-trimethylbenzenesulfonate (263 g, 0.638 moles) in MeOH (2630 mL, 10 mL/g) heated at 45° C. was charged K₂CO₃ (341 g, 1.28 moles) and stirred for 3 hours at 45° C. The mixture was then concentrated and the solids in water at 23° C. overnight, filtered and dried to give the title compound (89 g, 0.447 moles, 70%) as a brown solid. MS (ESI): mass calculated (calcd.) for C₇H₆BrN₃, 211.0. m/z found, 212.0 [M+H]⁺.

### Intermediate 2: N-(5-bromopyrazolo[1,5-a]pyridin-2-yl)cyclopropanecarboxamide

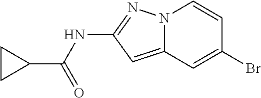 [SMILES: O=C(Nc1cc2cc(Br)ccn2n1)C1CC1]

A vessel containing 5-bromopyrazolo[1,5-a]pyridin-2-amine (Intermediate 1, 407 g, 1.45 mol) and DMA (8150 mL) was cooled to 10° C. Cyclopropanecarbonyl chloride (159 g, 1.53 mol) was added dropwise. The reaction mixture warmed to 20° C. and stirred for 1 h. Water (8 L) was added and the mixture was stirred overnight at room temperature. The resulting solid was isolated by filtration and dried under vacuum to provide the title compound as a tan solid (510 g, 95%). MS (ESI): mass calcd. for C₁₁H₁₀BrN₃O, 279.00. m/z found, 280.0/282.0 [M+H]⁺.

### Intermediate 3: N-(5-(4,4,5,5-Tetramethyl-1,3,2-dioxaborolan-2-yl)pyrazolo[1,5-a]pyridin-2-yl)cyclopropanecarboxamide

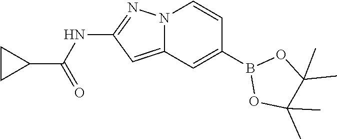 [SMILES: CC1(C)OB(c2ccn3nc(NC(=O)C4CC4)cc3c2)OC1(C)C]

To vessel containing N-(5-bromopyrazolo[1,5-a]pyridin-2-yl)cyclopropanecarboxamide (Intermediate 2, 300 g, 1.08 mol) and potassium acetate (211 g, 2.15 mol) were added 1,4-dioxane (3 L) and water (19.4 mL, 1.08 mmol). Nitrogen was bubbled through the mixture for 20 min, then bis(pinacolato)diboron (302 g, 1.19 mol) was added at room temperature. The reaction mixture was purged with N₂, heated at 70° C., charged with Pd(dppf)Cl₂ (39.5 g, 0.054 mol) and stirred for 2 h under N₂. The reaction mixture was cooled to rt, filtered, and the resulting solid was rinsed with EtOAc. The resulting solid was purified by column chromatography on silica gel using petroleum ether/ethyl acetate (50:1 to 10:1, gradient elution) to provide the title compound (230 g, 66%). MS (ESI): mass calcd. for C₁₇H₁₇BN₃O₃, 327.18. m/z found, 328.2 [M+H]⁺ and peak for corresponding boronic acid C₁₁H₁₂BN₃O₃ 246.1 [M+H]⁺.

### Intermediate 4: N-(5-(4-hydroxy-1-methyl-1H-pyrazol-5-yl)pyrazolo[1,5-a]pyridin-2-yl)cyclopropanecarboxamide

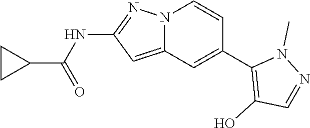 [SMILES: Cn1ncc(O)c1-c1ccn2nc(NC(=O)C3CC3)cc2c1]

A solid mixture consisting of N-(5-(4,4,5,5-tetramethyl-1,3,2-dioxaborolan-2-yl)pyrazolo[1,5-a]pyridin-2-yl)cyclopropanecarboxamide (Intermediate 3, 10.0 g, 30.5 mmol), 5-bromo-1-methyl-1H-pyrazol-4-ol (5.95 g, 33.6 mmol), Pd(dtbpf)Cl₂ (1990 mg, 3.06 mmol), and potassium fluoride (5.33 g, 91.7 mmol) contained in a round-bottomed flask was evacuated and back-filled with N₂ (3×). 1,4-dioxane (222 mL) and deionized water (47.0 mL) were added to the flask. The mixture was again evacuated and back-filled with N₂ (3×). The reaction was heated to 90° C. for 2 h. The crude reaction mixture was diluted with brine and EtOAc and the aq. layer was separated. The organic layer was dried over MgSO₄, filtered, and concentrated to dryness. The crude product was purified by FCC (eluent: hexanes:ethyl acetate=1:1 to 0:1). The desired fractions were combined resulting in a precipitate forming during concentration to afford a dark solid. The dark solid was triturated with ˜100 mL of EtOAc and heated to 50° C. The light brown precipitate was collected by suction filtration to afford the title compound (4.88 g, 54%) as a light brown solid. ¹H NMR (400 MHz, DMSO-d6) δ 11.05 (s, 1H), 8.80 (s, 1H), 8.57 (dt, J=7.3, 0.9 Hz, 1H), 7.66 (dd, J=2.0, 1.0 Hz, 1H), 7.14 (s, 1H), 6.98 (dd, J=7.2, 2.0 Hz, 1H), 6.87 (s, 1H), 3.82 (s, 3H), 1.93 (d, J=4.6 Hz, 1H), 0.83 (tt, J=7.9, 3.0 Hz, 4H).

### Intermediate 5: 5-(4,4,5,5-tetramethyl-1,3,2-dioxaborolan-2-yl)pyrazolo[1,5-a]pyridin-2-amine

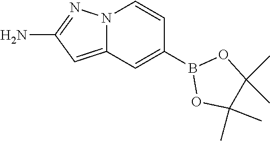 [SMILES: CC1(C)OB(c2ccn3nc(N)cc3c2)OC1(C)C]

To a 200 mL round-bottom flask was charged 5-bromopyrazolo[1,5-a]pyridin-2-amine (Intermediate 1, 5.10 g, 24.0 mmol), bis(pinacolato)diboron (12.5 g, 48.1 mmol), potassium acetate (7.08 g, 72.2 mmol), 1,1′-bis(diphenylphosphino)ferrocene-palladium(ii)dichloride dichloromethane complex (1.98 g, 2.41 mmol), and 1,4-dioxane (96 mL). The flask was capped with septum then evacuated and backfilled with N₂ several times. The flask was heated to 80° C. for 18 h. The reaction mixture was concentrated to dryness and purified directly by column chromatography on silica gel using hexanes/ethyl acetate (1:0 to 0:1; gradient elution) to provide the title compound (4.83 g, 77%). ¹H NMR (400 MHz, DMSO-d₆) δ 8.24-8.16 (m, 1H), 7.60 (t, J=1.2 Hz, 1H), 6.63 (dd, J=6.8, 1.4 Hz, 1H), 5.73-5.67 (m, 1H), 5.31 (s, 2H), 1.30 (s, 12H).

### Intermediate 6: tert-butyl (R)-2-(((5-(2-aminopyrazolo[1,5-a]pyridin-5-yl)-1-methyl-1H-pyrazol-4-yl)oxy)methyl)azetidine-1-carboxylate

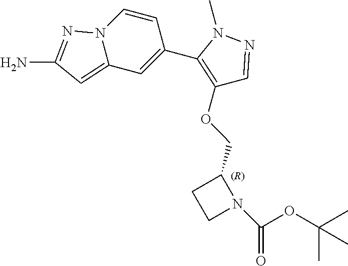

Step A: Tert-butyl (R)-2-(((1-methyl-1H-pyrazol-4-yl)oxy)methyl)azetidine-1-carboxylate. A 500 mL round bottom flask containing tert-butyl (R)-2-((tosyloxy)methyl)azetidine-1-carboxylate (22.2 g, 65.2 mmol) was added to DMSO (60 mL), followed by 1-methyl-1H-pyrazol-4-ol (9.81 g, 100 mmol) then cesium carbonate (32.7 g, 100 mmol). The reaction mixture was stirred for 16 h at room temperature, and LCMS showed partial conversion. An additional amount of 1-methyl-1H-pyrazol-4-ol (2.7 g) was added and the reaction was stirred for an additional 8 h at 45° C., after which LCMS showed complete conversion to the target product. The reaction mixture was diluted with water (500 mL). The product was extracted with DCM (2×500 mL). The combined organic layers were dried (MgSO₄), filtered, and concentrated to dryness. The residue was purified by flash column chromatography (eluent: ethyl acetate:hexanes=50-100%) to afford tert-butyl (R)-2-(((1-methyl-1H-pyrazol-4-yl)oxy)methyl)azetidine-1-carboxylate (13.8 g, 79%) as a clear oil.

Step B: Tert-butyl (R)-2-(((5-bromo-1-methyl-1H-pyrazol-4-yl)oxy)methyl)azetidine-1-carboxylate. A solution of tert-butyl (R)-2-(((1-methyl-1H-pyrazol-4-yl)oxy)methyl)azetidine-1-carboxylate (13.8 g, 51.7 mmol) in CH₃CN (120 mL) was stirred at room temperature and N-bromosuccinimde (NBS) (11.3 g, 63.5 mmol) was added in small portions over 15 min. Following addition of NBS, the reaction was found to be complete. The reaction mixture was diluted with EtOAc (200 mL) and extracted with aq. sodium thiosulfate (10 g in 200 mL water). The organic layer was washed with sat. aq. sodium bicarbonate. The organic layer was dried (MgSO₄) and concentrated to dryness. The residue was purified by flash column chromatography (eluent: ethyl acetate:hexanes=10-40%). Impure fractions were purified again under the same column conditions and combined to afford tert-butyl (R)-2-(((5-bromo-1-methyl-1H-pyrazol-4-yl)oxy)methyl)azetidine-1-carboxylate as a yellow oil (10.2 g, 57%).

Step C: Tert-butyl (R)-2-(((5-(2-aminopyrazolo[1,5-a]pyridin-5-yl)-1-methyl-1H-pyrazol-4-yl)oxy)methyl)azetidine-1-carboxylate. To a 40 mL vial was charged tert-butyl (R)-2-(((5-bromo-1-methyl-1H-pyrazol-4-yl)oxy)methyl)azetidine-1-carboxylate (500 mg, 1.44 mmol), Intermediate 5 (486 mg, 1.88 mmol), 1,1′-bis(diphenylphosphino)ferrocene-palladium(II) dichloride dichloromethane complex (119 mg, 0.144 mmol), 1,4-dioxane (5.8 mL), and 0.5 M aq. K₃PO₄ (2.9 mL). The vial was sealed then evacuated and backfilled with N₂ several times. The vial was heated to 90° C. for 1 h. The crude reaction mixture was diluted with ethyl acetate then washed with water and brine. The organics were separated, dried with MgSO₄, filtered then concentrated to dryness. The residue was purified directly by column chromatography on silica gel using hexanes/ethyl acetate (1:0 to 0:1; gradient elution) to provide the title compound (450 mg, 78%). ¹H NMR (500 MHz, Chloroform-d) δ 8.14 (d, J=7.1 Hz, 1H), 7.29-7.20 (m, 2H), 6.59 (dd, J=7.1, 2.0 Hz, 1H), 5.74 (d, J=5.1 Hz, 1H), 4.37-4.28 (m, 1H), 4.19 (dd, J=10.6, 4.7 Hz, 1H), 4.12 (s, 2H), 3.94 (dd, J=10.2, 2.7 Hz, 1H), 3.80 (s, 3H), 3.75-3.65 (m, 2H), 2.21-2.03 (m, 2H), 1.31 (d, J=5.3 Hz, 9H).

### Intermediate 7: (S)-5-(1-methyl-4-((1-methylpyrrolidin-3-yl)oxy)-1H-pyrazol-5-yl)pyrazolo[1,5-a]pyridin-2-amine

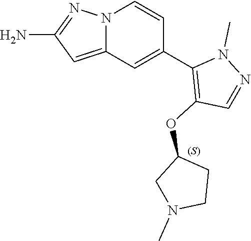

Step A: (R)-tert-butyl 3-(tosyloxy)pyrrolidine-1-carboxylate. A solution consisting of (R)-1-N-Boc-3-hydroxypyrrolidine (20.0 g, 107 mmol), TsCl (26.5 g, 139 mmol), DMAP (26.1 g, 214 mmol), and CH₂Cl₂ (200 mL) was stirred overnight at room-temperature. The reaction mixture was washed with 1N HCl (200 mL), sat. aqueous NaHCO₃ (200 mL), and brine (200 mL), dried over Na₂SO₄, filtered, and evaporated to dryness under reduced pressure to give the crude product (32 g, purity 90%, 79% yield) as a light-yellow oil. MS (ESI): mass calcd. for C₁₆H₂₃NO₅S, 341.13. m/z found, 286.1 [M+H−56]⁺.

Step B: (S)-tert-butyl 3-((1-methyl-1H-pyrazol-4-yl)oxy)pyrrolidine-1-carboxylate. To a mixture consisting of 1-methyl-1H-pyrazol-4-ol (5.5 g, 56 mmol), (R)-tert-butyl 3-(tosyloxy)pyrrolidine-1-carboxylate (22 g, 59 mmol) and DMF (100 mL) was added Cs₂CO₃ (54.8 g, 168 mmol) at 60° C. and stirred overnight. EtOAc (200 mL) and water (50 mL) were added and layers were separated. The aqueous phase was extracted with EtOAc (150 mL×2). Combined extracts were concentrated under vacuum to give a crude product, which was purified by FCC (eluent: PE:EA=0% to 50%) to afford the title product (14.1 g, 81% yield) as a yellow oil. MS (ESI): mass calcd. for C₁₃H₂₁N₃O₃, 267.16. m/z found, 212.2 [M+H−56]⁺.

Step C: (S)-tert-butyl 3-((5-bromo-1-methyl-1H-pyrazol-4-yl)oxy)pyrrolidine-1-carboxylate. A solution consisting of (S)-tert-butyl 3-((1-methyl-1H-pyrazol-4-yl)oxy)pyrrolidine-1-carboxylate (14 g, 52 mmol), NBS (9.3 g, 52 mmol), and ACN (100 mL) was stirred at room-temperature for 3 h. The reaction mixture was concentrated under vacuum to give a residue, which was purified by FCC (eluent: PE:EA=0% to 30%) to afford the title product (14.36 g, 75% yield) as a yellow oil. MS (ESI): mass calcd. for C₁₃H₂₀BrN₃O₃, 345.07. m/z found, 292.0 [M+H−56]⁺.

Step D: (S)-tert-butyl 3-((5-(2-((tert-butoxycarbonyl)amino)pyrazolo[1,5-a]pyridin-5-yl)-1-methyl-1H-pyrazol-4-yl)oxy)pyrrolidine-1-carboxylate. A mixture consisting of tert-butyl (5-(4,4,5,5-tetramethyl-1,3,2-dioxaborolan-2-yl)pyrazolo[1,5-a]pyridin-2-yl)carbamate (14 g, 39 mmol), (S)-tert-butyl 3-((5-bromo-1-methyl-1H-pyrazol-4-yl)oxy)pyrrolidine-1-carboxylate (14.2 g, 40.9 mmol), Pd(dtbpf)Cl₂ (1.27 g, 1.95 mmol), CsF (17.8 g, 117 mmol), and 1,4-dioxane:H₂O (4:1) (200 mL) was stirred at 80° C. for 2 h under N₂. The reaction mixture was cooled to room temperature and concentrated under vacuum to give a residue, which was purified by FCC (eluent: PE:EA=20% to 60%) to afford the title product (26 g, crude) as a yellow oil. MS (ESI): mass calcd. for C₂₅H₃₄N₆O₅, 498.26. m/z found, 499.4 [M+H]⁺.

Step E: (S)-5-(1-methyl-4-(pyrrolidin-3-yloxy)-1H-pyrazol-5-yl)pyrazolo[1,5-a]pyridin-2-amine. A mixture consisting of (S)-tert-butyl 3-((5-(2-((tert-butoxycarbonyl)amino)pyrazolo[1,5-a]pyridin-5-yl)-1-methyl-1H-pyrazol-4-yl)oxy)pyrrolidine-1-carboxylate (26 g, 40 mmol) and HCl/1,4-dioxane (200 mL, 4 M) was stirred at room-temperature for 2 h. The reaction mixture was concentrated under vacuum to give a crude product. EtOAc (500 mL) and MeOH (20 mL) were added and stirred at room-temperature for 15 min. During this period, yellow precipitate was formed. It was collected by filtration, which was basified with NaHCO₃ to pH=8, then purified by FCC (6% TEA in eluent: CH₂Cl₂:(EA:EtOH=3:1) 0% to 80%) to afford the title product (9.2 g, 81% yield) as a yellow oil. MS (ESI): mass calcd. for C₁₅H₁₈N₆O, 298.15. m/z found, 299.2 [M+H]⁺.

Step F: (S)-5-(1-methyl-4-((1-methylpyrrolidin-3-yl)oxy)-1H-pyrazol-5-yl)pyrazolo[1,5-a]pyridin-2-amine. To a solution of (S)-5-(1-methyl-4-(pyrrolidin-3-yloxy)-1H-pyrazol-5-yl)pyrazolo[1,5-a]pyridin-2-amine (8.30 g, 27.8 mmol) and H(CHO)n (1.0 g, 11 mmol) in MeOH (50 mL) was added NaBH₃CN (2.62 g, 41.7 mmol) at room-temperature and stirred for 50 min.

The pH was adjusted to 6-7 with 1M aq. HCl. The solution was purified by preparative HPLC using a Phenomenex prep L-Y YMC-Triart Prep C18 250×50 mm×10 um column (eluent: 0% to 20.5% (v/v) water (0.05% HCl)-ACN). The pure fractions were concentrated under reduced pressure. The aqueous phase was diluted with MeOH (30 mL). OH-resin (Alfa Aesar Ambersep 900 (OH) ion exchange resin) was added until pH ˜8 and the resultant mixture was stirred for 0.5 h. The insoluble resin was filtered off and the filtrate was concentrated under reduced pressure. The remaining aqueous phase was lyophilized to dryness to afford the title compound (4.1 g, 39% yield) as a yellow solid. MS (ESI): mass calcd. for C₁₆H₂₀N₆O, 312.17. m/z found, 313.2 [M+H]⁺.

### Intermediate 8: tert-Butyl (2R,3R)-2-(hydroxymethyl)-3-methylazetidine-1-carboxylate

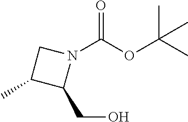

Step A: 2-Bromo-3-(((tert-butyldimethylsilyl)oxy)methyl)pyridine. To a 0° C. mixture of (2-bromopyridin-3-yl)methanol (1.20 g, 6.38 mmol) in DCM (11 mL) and imidazole (1.30 g, 19.1 mmol) was added tert-butylchlorodimethylsilane (1.25 g, 8.30 mmol). The reaction was stirred for 2 h at room temperature. The crude product was diluted with 20 mL of DCM and 20 mL of H₂O. The layers were separated. The organic layer was washed with H₂O (20 mL) and then dried over MgSO₄, filtered, and concentrated down under vacuo. The crude was purified by flash column chromatography (eluent: 5-50% EtOAc/hexanes) to afford the title compound (1.81 g, 94%). MS (ESI): mass calcd. for C₁₂H₂₀BrNOSi, 301.1. m/z found, 302.1 [M+H]⁺. ¹H NMR (400 MHz, Chloroform-d) δ 8.25 (ddt, J=4.8, 2.0, 0.9 Hz, 1H), 7.85 (ddt, J=7.6, 2.1, 1.1 Hz, 1H), 7.30 (dd, J=7.6, 4.7 Hz, 1H), 4.69 (d, J=1.0 Hz, 2H), 0.97 (s, 9H), 0.15 (s, 6H).

Step B: Benzyl 3-(((tert-butyldimethylsilyl)oxy)methyl)picolinate. 2-Bromo-3-(((tert-butyldimethylsilyl)oxy)methyl)pyridine (1.20 g, 3.97 mmol), triethylamine (2.76 mL, 19.8 mmol), (dppf)PdCl₂ (0.29 g, 0.397 mmol) and DMF (5.53 mL, 71.5 mmol) were added into a stainless-steel reaction vessel. The container was sealed and filled with CO to 4 bar. The reaction mixture was heated at 100° C. with stirring for 16 hours. Water (50 mL) was added to the reaction mixture. The mixture was extracted with EtOAc (50 mL×3). The combined extracts were washed with brine and concentrated to dryness under reduced pressure to afford the crude title product, which was purified by flash column chromatography (FCC) (eluent: 10% EtOAc/hexanes) to afford a yellow oil (1.23 g, 86.7%). MS (ESI): mass calcd. for C₂₀H₂₇NO₃Si, 357.2. m/z found, 358.2 [M+H]⁺. ¹H NMR (500 MHz, Chloroform-d) δ 8.62 (ddt, J=4.7, 1.7, 0.8 Hz, 1H), 8.18 (ddt, J=8.0, 1.9, 1.0 Hz, 1H), 7.52-7.44 (m, 3H), 7.41-7.28 (m, 3H), 5.44 (s, 2H), 5.07 (q, J=0.8 Hz, 2H), 0.95 (s, 9H), 0.10 (s, 6H).

Step C: 3-(((tert-Butyldimethylsilyl)oxy)methyl)picolinic acid. Benzyl 3-(((tert-butyldimethylsilyl)oxy)methyl)picolinate (1.20 g, 3.36 mmol), Pd/C (252 mg, 0.71 mmol) and EtOAc (17.5 mL) were added to a 250 mL round-bottomed flask. The reaction mixture was stirred under H₂ (1 atm) at 25° C. for 2 hours. LCMS showed the reaction was complete. The mixture was filtered. The filtrate was concentrated to dryness under vacuum to afford the product as a white solid (0.68 g, 76% yield), which was used without further purification. MS (ESI): mass calcd. for C₁₃H₂₁NO₃Si, 267.1. m/z found, 268.2 [M+H]⁺. ¹H NMR (400 MHz, DMSO-d6) δ 12.98 (s, 1H), 8.46 (ddd, J=4.6, 1.6, 0.8 Hz, 1H), 7.97 (ddt, J=7.9, 1.8, 1.0 Hz, 1H), 7.54 (dd, J=7.9, 4.7 Hz, 1H), 4.91 (s, 2H), 0.82 (s, 9H).

Step D: methyl (3-(((tert-butyldimethylsilyl)oxy)methyl)picolinoyl)-D-valinate. HATU (3.40 g, 8.95 mmol) was added to a solution consisting of 3-(((tert-butyldimethylsilyl)oxy)methyl)picolinic acid (2.39 g, 7.16 mmol), methyl D-valinate (1.00 g, 5.97 mmol), DIPEA (5.21 mL, 29.8 mmol) and DCM (23 mL). The reaction mixture was stirred at room-temperature for 2 hours. The reaction mixture was washed with H₂O and concentrated to dryness under reduced pressure to give the crude product, which was purified by FCC (eluent: 5-50% EtOAc/hexanes) to afford the title compound as clear oil (1.69 g, 74%). MS (ESI): mass calcd. for C₁₉H₃₂NO₄Si, 380.2. m/z found, 381.2 [M+H]⁺. ¹H NMR (500 MHz, Chloroform-d) δ 8.57 (d, J=9.2 Hz, 1H), 8.33 (ddt, J=4.6, 1.7, 0.9 Hz, 1H), 8.12 (ddt, J=8.0, 1.9, 1.1 Hz, 1H), 7.34 (dd, J=8.0, 4.5 Hz, 1H), 5.16 (dq, J=1.7, 0.8 Hz, 2H), 4.51 (dd, J=9.2, 5.2 Hz, 1H), 3.63 (s, 3H), 2.16 (pd, J=6.9, 5.2 Hz, 1H), 0.89 (d, J=6.9 Hz, 6H), 0.84 (s, 9H).

Step E: Methyl (2R,3R)-1-(3-(((tert-butyldimethylsilyl)oxy)methyl)picolinoyl)-3-methylazetidine-2-carboxylate. To 2 microwave vials, each was added Pd(OAc)₂ (11.8 mg, 0.0526 mmol) and phenyl-□³-iodanediyl diacetate (846 mg, 2.63 mmol). The vial was evacuated and back-filled with N₂ 3 times. To the solid mixture was added a pre-dissolved solution of methyl (3-(((tert-butyldimethylsilyl)oxy)methyl)picolinoyl)-D-valinate (400 mg, 1.05 mmol) and toluene (10.3 mL). The mixture was evacuated and backfilled with N₂ 5 times followed by AcOH (0.12 mL, 2.10 mmol). The vial was heated to 110° C. in the microwave for 2 hours. LCMS showed the reaction was complete. The crude reaction solution was concentrated down under vacuo, then, redissolved with EtOAc and absorbed onto silica gel. The crude product was flashed with 10-30% EtOAc/hexanes to afford 110 mg of clear oil. The mixed fractions were flashed again with 5-20% EtOAc/hexanes to afford 75 mg more of clear oil. The combined yield was 46%. MS (ESI): mass calcd. for C₁₉H₃₀N₂O₄Si, 378.2. m/z found, 379.2 [M+H]⁺.

Step F: Methyl (2R,3R)-3-methylazetidine-2-carboxylate. Methyl (2R,3R)-1-(3-(((tert-butyldimethylsilyl)oxy)methyl)picolinoyl)-3-methylazetidine-2-carboxylate (110 mg, 0.291 mmol) was added to a 50 mL round-bottomed flask and the resulting mixture was dissolved in 1,4-dioxane (5 mL). HCl (5 mL) was added. The mixture was then stirred at room temperature for 2 days. The mixture was concentrated to dryness under vacuum to afford the crude title product (30 mg), which was used without further purification. MS (ESI): mass calcd. for C₆H₁₁NO₂, 129.2. m/z found, 130.2 [M+H]⁺.

Step G: 1-(tert-butyl) 2-methyl (2R,3R)-3-methylazetidine-1,2-dicarboxylate. Boc₂O (242 mg, 1.11 mmol) was added to a mixture consisting of methyl (2R,3R)-3-methylazetidine-2-carboxylate (92 mg, 0.56 mmol), sodium carbonate (589 mg, 5.55 mmol), water (0.1 mL) and 1,4-dioxane (0.95 mL). The resultant reaction mixture was stirred at room temperature overnight. Water (20 mL) was added to the reaction mixture. The resulting mixture was extracted with EtOAc (40 mL×3). The combined organic layer was dried over anhydrous MgSO₄, filtered and concentrated to dryness under vacuum to afford the crude title product, which was purified by FCC (10-100% EtOAc/hexanes) to afford product (110 mg, 86%) as a colorless oil. MS (ESI): mass calcd. for C₁₁H₁₉NO₄, 229.1. m/z found, [M+H]⁺. ¹H NMR (500 MHz, Chloroform-d) δ 4.18 (d, J=5.0 Hz, 1H), 4.11 (t, J=8.0 Hz, 1H), 3.76 (s, 3H), 3.45 (dd, J=7.9, 5.2 Hz, 1H), 2.62-2.50 (m, 1H), 1.42 (s, 9H), 1.32 (d, J=6.9 Hz, 3H).

Step H: tert-Butyl (2R,3R)-2-(hydroxymethyl)-3-methylazetidine-1-carboxylate. To a 10 mL microwave vial was added 1-(tert-butyl) 2-methyl (2R,3R)-3-methylazetidine-1,2-dicarboxylate (225 mg, 0.981 mmol) and THE (9.6 mL) and then NaBH₄ (186 mg, 4.91 mmol). The reaction was heated to 40° C. for 2 days and more NaBH₄ (186 mg, 4.91 mmol) was added. LCMS showed the reaction was complete. The crude product was diluted with DCM (50 mL) and quenched with sat. aq. Rochelle's salt. The bilayers were shaken vigorously in a separation funnel and 2 clear layers formed. The layers were separated. The organic layer was collected. The aq. layer was extracted with DCM and with EtOAc. The combined organic layers were dried over anh. MgSO₄, filtered, and concentrated down to afford a clear oil (190 mg, 96%). This material was used as is. MS (ESI): mass calcd. for C₁₀H₁₉NO₃, 201.1. m/z found, [M+H]⁺. ¹H NMR (400 MHz, Chloroform-d) δ 4.42-4.08 (m, 1H), 4.00 (s, 1H), 3.91 (t, J=8.3 Hz, 1H), 3.81-3.65 (m, 2H), 3.47 (dd, J=8.3, 6.5 Hz, 1H), 2.29 (s, 1H), 1.48 (s, 9H), 1.21 (d, J=6.9 Hz, 3H).

### Intermediate 9. 5-[2-methyl-4-[[(2R)-1-methylazetidin-2-yl]methoxy]pyrazol-3-yl]pyrazolo[1,5-a]pyridin-2-amine

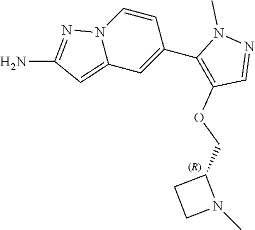

Step A: Tert-butyl (R)-2-(((1-methyl-1H-pyrazol-4-yl)oxy)methyl)azetidine-1-carboxylate. To a rounded-bottom flask was charged tert-butyl (R)-2-((tosyloxy)methyl)azetidine-1-carboxylate (9.00 g, 26.4 mmol), 1-methyl-1H-pyrazol-4-ol (2.59 g, 26.4 mmol), cesium carbonate (25.8 g, 79.1 mmol), and DMF (100 mL). The vial was heated at 60° C. for 18 h. The mixture was filtered and concentrated in-vacuo to give the crude product. The crude material was purified by silica gel chromatography (0-60% EtOAc in hexanes) to give tert-butyl (R)-2-(((1-methyl-1H-pyrazol-4-yl)oxy)methyl)azetidine-1-carboxylate as a yellow oil (6.50 g, 92%) which was used as is in the following reaction.

Step B: Tert-butyl (R)-2-(((5-bromo-1-methyl-1H-pyrazol-4-yl)oxy)methyl)azetidine-1-carboxylate. To a round-bottom flask was charged tert-butyl (R)-2-(((1-methyl-1H-pyrazol-4-yl)oxy)methyl)azetidine-1-carboxylate (6.50 g, 24.3 mmol) and acetonitrile (100 mL). N-bromo succinimide (4.76 g, 26.7 mmol) was added portion wise to the flask. After 3 hours at 23° C., the mixture was concentrated under vacuum to give the crude product. The crude material was purified by silica gel chromatography (0-100% EtOAc in hexanes) to give tert-butyl (R)-2-(((5-bromo-1-methyl-1H-pyrazol-4-yl)oxy)methyl)azetidine-1-carboxylate (8.30 g, 23.9 mmol, 98%) as a yellow oil which was used as is in the following reaction.

Step C: (R)-4-(azetidin-2-ylmethoxy)-5-bromo-1-methyl-1H-pyrazole. To a solution of tert-butyl (R)-2-(((5-bromo-1-methyl-1H-pyrazol-4-yl)oxy)methyl)azetidine-1-carboxylate (1.00 g, 2.89 mmol) in DCM (10 mL) was added trifluoracetic acid (5.0 mL, 29.0 mmol) at 23° C. After 30 minutes the reaction mixture was concentrated in vacuo and purified by silica gel chromatography (0-10% 2M NH₃ in MeOH in DCM) to give (R)-4-(azetidin-2-ylmethoxy)-5-bromo-1-methyl-1H-pyrazole (700 mg, 2.8 mmol, 98%) as a brown oil which was used as is in the following reaction.

Step D: (R)-5-bromo-1-methyl-4-((1-methylazetidin-2-yl)methoxy)-1H-pyrazole. To a solution of (R)-4-(azetidin-2-ylmethoxy)-5-bromo-1-methyl-1H-pyrazole (700 mg, 2.84 mmol), paraformaldehyde (167 mg, 1.85 mmol) in MeOH (10 mL) was added sodium cyanoborohydride (322 mg, 5.12 mmol) at 23° C. After 2 hours, the mixture was concentrated in vacuo to give the crude product as a brown oil. The crude mixture was then purified by silica gel chromatography (0-10% 2M NH₃ in MeOH in DCM) to give (R)-5-bromo-1-methyl-4-((1-methylazetidin-2-yl)methoxy)-1H-pyrazole (650 mg, 2.50 mmol, 88%) as a white solid which was used as is in the following reaction.

Step E: 5-[2-methyl-4-[[(2R)-1-methylazetidin-2-yl]methoxy]pyrazol-3-yl]pyrazolo[1,5-a]pyridin-2-amine. To a microwave vial containing (R)-5-bromo-1-methyl-4-((1-methylazetidin-2-yl)methoxy)-1H-pyrazole (250 mg, 0.96 mmol), Intermediate 5 (323 mg, 1.25 mmol), 1,1′-bis(diphenylphosphino)ferrocene-palladium(ii)dichloride dichloromethane complex (79 mg, 0.096 mmol), 1,4-dioxane (3.8 mL) and 0.5 M aq. K₃PO₄ (1.9 mL) was evacuated and backfilled with N₂ and then heated in the microwave at 90° C. for 1 hour. The reaction mixture was then diluted with water (20 mL) and extracted with DCM (3×30 mL). The combined organic layers were dried with anhydrous MgSO₄, filtered and then concentrated in vacuo. The crude residue was purified by silica gel chromatography (50-100% EtOAc in hexanes) to provide the title compound as a yellow foam (100 mg, 0.32 mmol, 33%). MS (ESI): mass calcd. for C₁₆H₂₀N₆O, 312.375. m/z found, 313.20 [M+H]⁺. ¹H NMR (500 MHz, Chloroform-d) δ 8.12 (dt, J=7.1, 0.9 Hz, 1H), 7.27-7.19 (m, 2H), 6.59 (dd, J=7.1, 1.9 Hz, 1H), 5.72 (d, J=0.8 Hz, 1H), 4.00 (s, 2H), 3.87 (d, J=5.6 Hz, 2H), 3.80 (s, 3H), 3.39 (s, OH), 3.35-3.29 (m, 1H), 3.24-3.18 m, 1H), 2.76-2.69 (m, 1H), 2.23 (s, 3H), 1.97-1.82 (m, 2H).

### Intermediate 10. 5-(2-((2,6-dimethylpyrimidin-4-yl)amino)pyrazolo[1,5-a]pyridin-5-yl)-1-methyl-1H-pyrazol-4-ol

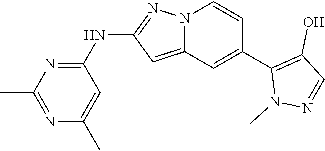 [SMILES: Cc1cc(Nc2cc3cc(-c4c(O)cnn4C)ccn3n2)nc(C)n1]

Step A: 4-((tert-butyldimethylsilyl)oxy)-1-methyl-1H-pyrazole. TBSCl (39.9 g, 265 mml) was added to a solution consisting of 1-methyl-1H-pyrazol-4-ol (20.0 g, 204 mmol), imidazole (41.6 g, 612 mmol), and CH₂Cl₂ (500 mL). The reaction mixture was stirred at room-temperature for 5 h. The reaction mixture was treated with water (200 mL), and extracted with CH₂Cl₂ (500 mL×2), the combined organic phases was washed with H₂O (200 mL×2), dried over anhydrous Na₂SO₄, filtered, and concentrated to dryness under reduced pressure to give the title compound (57 g, crude) as a yellow oil.

Step B: 5-bromo-4-((tert-butyldimethylsilyl)oxy)-1-methyl-1H-pyrazole. NBS (52.5 g, 295 mmol) was in portions added to a solution consisting of 4-((tert-butyldimethylsilyl)oxy)-1-methyl-1H-pyrazole (57 g, 268 mmol) and CH₃CN (500 mL), and then the reaction mixture was stirred at room-temperature for 2 h. The reaction mixture was concentrated under reduced pressure to give the crude product, to which was added H₂O (200 mL) and extracted with EtOAc (500 mL×2). The combined organic phases were washed with H₂O (150 mL) and sat. Na₂SO₃ (100 mL×2). The organic layer was dried over Na₂SO₄, filtered and concentrated to give the title product (63 g, crude) as a yellow oil, which was used next in the step without further purification.

Step C: 5-(4-((tert-butyldimethylsilyl)oxy)-1-methyl-1H-pyrazol-5-yl)pyrazolo[1,5-a]pyridin-2-amine. A mixture consisting of 5-(4,4,5,5-tetramethyl-1,3,2-dioxaborolan-2-yl)pyrazolo[1,5-a]pyridin-2-amine (10 g, 39 mmol), 5-bromo-4-((tert-butyldimethylsilyl)oxy)-1-methyl-1H-pyrazole (17 g, 58 mmol), Pd(dppf)Cl₂ (2.8 g, 3.9 mmol), Na₂CO₃ (12.3 g, 116 mmol), 1,4-dioxane (160 mL) and H₂O (40 mL) was stirred at 80° C. for 4 h under N₂. The reaction mixture was filtered, and the filtrate was concentrated to dryness under reduced pressure to afford the crude product, which was purified by FCC (eluent: PE:EA=1:0 to 0:1) afford the title product (12 g, 31%) as a yellow oil.

Step D: 5-(4-((tert-butyldimethylsilyl)oxy)-1-methyl-1H-pyrazol-5-yl)-N-(2,6-dimethylpyrimidin-4-yl)pyrazolo[1,5-a]pyridin-2-amine. To a mixture consisting of 5-(4-((tert-butyldimethylsilyl)oxy)-1-methyl-1H-pyrazol-5-yl)pyrazolo[1,5-a]pyridin-2-amine (11.9 g, 24.8 mmol), 4-chloro-2,6-dimethylpyrimidine (5.1 g, 36 mmol) and Cs₂CO₃ (27 g, 83 mmol) in 1,4-dioxane (190 mL) was added BrettPhos Pd G3 (2.5 g, 2.8 mmol) under N₂ atmosphere. The resulting mixture was stirred at 50° C. for 4 h. The mixture was filtered and concentrated to dryness under reduced pressure to give the crude product (23 g, crude) as a yellow oil, which was used for next step directly.

Step E: 5-(2-((2,6-dimethylpyrimidin-4-yl)amino)pyrazolo[1,5-a]pyridin-5-yl)-1-methyl-1H-pyrazol-4-ol. A mixture of N-(5-(4-((tert-butyldimethylsilyl)oxy)-1-methyl-1H-pyrazol-5-yl)pyrazolo[1,5-a]pyridin-2-yl)cyclopropanecarboxamide (23 g, 51 mmol) in 1,4-dioxane/H₂O (250 mL) was added CsF (38.9 g, 256 mmol), then the mixture was stirred at 65° C. for 12 h. The reactant mixture was poured into water (100 mL) and extracted with ethyl acetate (200 mL×3). The organic extracts were washed with brine (100 mL×1), dried over anh. Na₂SO₄, filtered, and concentrated to dryness under reduced pressure to obtain the crude product, which was purified by FCC (eluent: petroleum ether:ethyl acetate=1:0 to 0:1) to afford the title compound (7 g, 41%) as a yellow solid.

### Intermediate 11: N-(2,6-dimethylpyrimidin-4-yl)-5-(4,4,5,5-tetramethyl-1,3,2-dioxaborolan-2-yl)pyrazolo[1,5-a]pyridin-2-amine

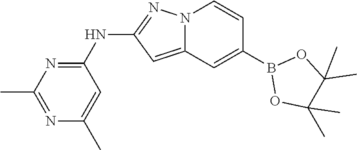

Into a 2 L 4-necked round-bottom flask purged and maintained with an inert atmosphere of nitrogen, was placed Intermediate 5 (65.00 g, 250.00 mmol), 4-chloro-2,6-dimethylpyrimidine (67.70 g, 480.00 mmol), t-BuBrettPhos Pd G3 (10.50 g, 12.50 mmol), Cs₂CO₃ (162.50 g, 500.00 mmol), 1,4-dioxane (975.00 mL). The resulting solution was stirred for 5 h at 90° C. The reaction mixture was cooled to 25° C. with a water/ice bath. The solids were filtered out. The filtrate was then quenched by the addition of 2 L of water/ice. The resulting solution was extracted with 3×1 L of ethyl acetate and the organic layer was concentrated. The residue was applied onto a silica gel column with ethyl acetate/petroleum ether (1:10). This resulted in 50.3 g (54.9%) of the title compound as a white solid. MS (ESI): mass calcd. for C₁₉H₂₄BN₅O₂, 365.20. m/z found, 366.2 [M+H]⁺. ¹H NMR (300 MHz, DMSO-d₆, ppm) δ 10.21 (s, 1H), 8.50 (d, J=6.9 Hz, 1H), 7.88 (s, 1H), 7.01 (s, 1H), 6.89-6.86 (m, 2H), 2.46 (s, 3H), 2.30 (s, 3H), 1.32 (s, 12H).

### Intermediate 12: tert-butyl (R)-2-(((5-(2-aminopyrazolo[1,5-a]pyridin-5-yl)-1-(difluoromethyl)-1H-pyrazol-4-yl)oxy)methyl)azetidine-1-carboxylate

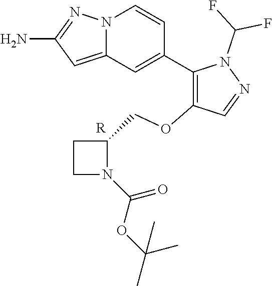

Step A: tert-butyl (R)-2-(((1H-pyrazol-4-yl)oxy)methyl)azetidine-1-carboxylate. A solution containing 1H-pyrazol-4-ol (2.20 g, 26.2 mmol), potassium carbonate (4.34 g, 31.4 mmol), tert-butyl (R)-2-((tosyloxy)methyl)azetidine-1-carboxylate (8.93 g, 26.2 mmol) in DMF (30 mL) was heated at 80° C. for 16 hours. Additional K₂CO₃ (1.10 g, 7.87 mmol) and stirred for an additional 3 hours before cooling to 23° C. The reaction mixture was diluted with water (30 mL) and extracted with EtOAc (3×30 mL). The combined organics were washed with sat. aq. NaCl (2×20 mL), dried with MgSO₄, filtered and concentrated in vacuo. The resulting oil was purified by silica gel chromatography (10-100%) EtOAc in hexanes to provide the titled compound (3.64 g, 54%) which was used as in the following reaction.

Step B: tert-butyl (R)-2-(((1-(difluoromethyl)-1H-pyrazol-4-yl)oxy)methyl)azetidine-1-carboxylate. A solution of tert-butyl (R)-2-(((1H-pyrazol-4-yl)oxy)methyl)azetidine-1-carboxylate (4.20 g, 16.9 mmol), KF (1.93 g, 33.2 mmol) and diethyl (bromodifluoromethyl)phosphonate (4.87 g, 18.2 mmol) in acetonitrile (86 mL) was added diethyl (bromodifluoromethyl)phosphonate (4.87 g, 18.2 mmol) dropwise by syringe. The resulting mixture was stirred at 23° C. for 16 hours and then filtered and the organics concentrated in vacuo. The crude yellow oil was purified by silica gel chromatography (0-10%) MeOH in DCM to give the titled compound (4.91 g, 97%) as a pale-yellow oil which was used as is in the following reaction.

Step C: tert-butyl (R)-2-(((5-bromo-1-(difluoromethyl)-1H-pyrazol-4-yl)oxy)methyl)azetidine-1-carboxylate. A solution of tert-butyl (R)-2-(((1-(difluoromethyl)-1H-pyrazol-4-yl)oxy)methyl)azetidine-1-carboxylate (5.00 g, 16.5 mmol) in acetonitrile (150 mL) was added N-bromosuccinimide (3.23 g, 18.1 mmol) and the resulting mixture stirred at 50° C. for 16 hours. Additional N-bromosuccinimide (1.75 g, 9.81 mmol) was added and stirred for an additional 8 hours at 50° C. The mixture was then concentrated in-vacuo and purified by silica gel chromatography (0-100%) EtOAc in hexanes to give the title compound (4.75 g, 75%) as yellow oil which was used as is in the following reaction.

Step D: tert-butyl (R)-2-(((5-(2-aminopyrazolo[1,5-a]pyridin-5-yl)-1-(difluoromethyl)-1H-pyrazol-4-yl)oxy)methyl)azetidine-1-carboxylate. To a vial was charged tert-butyl (R)-2-(((5-bromo-1-(difluoromethyl)-1H-pyrazol-4-yl)oxy)methyl)azetidine-1-carboxylate (2.00 g, 5.23 mmol), Intermediate 5 (1.42 g, 5.50 mmol), 1,1′-bis(diphenylphosphino)ferrocene-palladium(II)dichloride dichloromethane complex (383 mg, 0.52 mmol), 1,4-dioxane (18 mL), and 0.5 M aq. K₃PO₄ (15 mL). The vial was sealed then evacuated and backfilled with N₂ several times. The vial was heated to 60° C. for 18 h. The reaction as cooled to 23° C. and diluted with water (30 mL) and extracted with 10% MeOH in DCM (4×30 mL). The combined organics were dried with MgSO₄, filtered then concentrated to dryness. The residue was purified by silica column chromatography (0-100%) using EtOAc in hexanes to afford the titled compound (984 mg, 43%). MS (ESI): mass calcd. for C₂₀H₂₄F₂N₆O₂, 434.19. m/z found, 435.2 [M+H]⁺. ¹H NMR (400 MHz, DMSO-d6) δ 8.40-8.23 (m, 1H), 7.89 (s, 1H), 7.56-7.43 (m, 2H), 6.61-6.48 (m, 1H), 5.78-5.65 (m, 1H), 5.38 (s, 2H), 4.37-4.25 (m, 2H), 4.09-4.05 (m, 2H), 3.67-3.54 (m, 2H), 2.24-2.12 (m, 1H), 2.08-1.95 (m, 1H), 1.25 (s, 9H).

### Example 1: N-(5-(4-(((3S,5S)-1,5-dimethylpyrrolidin-3-yl)oxy)-1-methyl-1H-pyrazol-5-yl)pyrazolo[1,5-a]pyridin-2-yl)cyclopropanecarboxamide

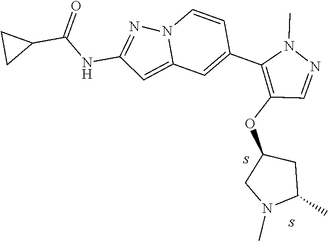

Step A: Tert-butyl (2S,4R)-2-methyl-4-(tosyloxy)pyrrolidine-1-carboxylate. To a vial containing (2S,4R)-tert-butyl 4-hydroxy-2-methylpyrrolidine-1-carboxylate (1 g, 4.97 mmol) was added 4-dimethylaminopyridine (910 mg, 7.45 mmol) and dichloromethane (10 mL, 0.5 M). The vial was placed in an ice-bath, and p-toluenesulfonyl chloride (1.14 g, 5.96 mmol) was added while stirring. The vial was warmed to room temperature for 3.5 hr. The solution was concentrated and adsorbed onto silica gel, then purified via FCC (0-100% EtOAc/hexanes) to provide the title compound a clear oil (1.77 g, 89%).

Step B: Tert-butyl (2S,4S)-2-methyl-4-((1-methyl-1H-pyrazol-4-yl)oxy)pyrrolidine-1-carboxylate. To a vial containing (2S,4R)-2-methyl-4-(tosyloxy)pyrrolidine-1-carboxylate (1.57 g, 4.40 mmol) was added 1-methyl-1h-pyrazol-4-ol (540 mg, 5.50 mmol), cesium carbonate (2.87 g, 8.80 mmol), and DMF (8.80 mL, 0.5 M). The reaction was stirred for 20 h, then extracted with EtOAc/water/brine. The organic layer was separated, dried with sodium sulfate, then purified via FCC (25-100% EtOAc/hexanes) to give the title compound as a yellow oil (975 mg, 79%).

Step C: Tert-butyl (2S,4S)-4-((5-bromo-1-methyl-1H-pyrazol-4-yl)oxy)-2-methylpyrrolidine-1-carboxylate. To a vial containing tert-butyl (2S,4S)-2-methyl-4-((1-methyl-1H-pyrazol-4-yl)oxy)pyrrolidine-1-carboxylate (975 mg, 3.47 mmol) was added acetonitrile (17.3 mL, 0.2 M). NBS (617 mg, 3.47 mmol) was added portion-wisely. The reaction was quenched with saturated sodium thiosulfate, then extracted with DCM (water and brine was also added to the separatory funnel). The organic extracts were dried with sodium sulfate, then purified via FCC (25-100% EtOAc/hexanes) to obtain the title compound as a yellow oil (706 mg, 57%).

Step D: tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate. To a vial containing tert-butyl (2S,4S)-4-((5-bromo-1-methyl-1H-pyrazol-4-yl)oxy)-2-methylpyrrolidine-1-carboxylate (361 mg, 1.00 mmol) was added N-[5-(4,4,5,5-tetramethyl-1,3,2-dioxaborolan-2-yl)pyrazolo[1,5-a]pyridin-2-yl]cyclopropanecarboxamide (Intermediate 3, 394 mg, 1.20 mmol), 1,4-dioxane (4 mL, 0.25 M), and 0.5 M aq. K₃PO₄ (2 mL, 0.5 M). The mixture was stirred, then 1,1′-bis(diphenylphosphino)ferrocene-palladium(II)dichloride dichloromethane complex (83 mg, 0.1 mmol) was added. The reaction was evacuated and back-filled with nitrogen, then stirred at 90° C. for 2.5 hr. The mixture was worked-up with EtOAc/water/brine, and the extracted organics dried over sodium sulfate. The crude was purified via FCC (0-20% 2M NH₃ in MeOH/DCM) to provide the title compound as a brown solid (400 mg, 83%).

Step E: N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. To a vial containing tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate (400 mg, 0.83 mmol) was added DCE (3.33 mL, 0.25 M) and TFA (0.64 mL, 8.32 mmol). The reaction was stirred for 16 h, at room temp. The mixture was concentrated to dryness, diluted with 200 mL 10% IPA/chloroform, then washed with 10 mL sat. NaHCO₃+5 mL brine+5 mL H₂O. The aq. layer was back-extracted with another 200 mL 10% IPA/chloroform. The combined organics were dried over anhydrous Na₂SO₄ and purified via FCC (0-20% 2M NH₃ MeOH/DCM) to provide the title compound as a beige solid (281 mg, 89%). MS (ESI): mass calcd. for C₂₀H₂₄N₆O₂, 380.2. m/z found, 381.2 [M+H]⁺. ¹H NMR (500 MHz, Methanol-d4) δ 8.48-8.42 (m, 1H), 7.64-7.58 (m, 1H), 7.38 (s, 1H), 6.93 (dd, J=7.2, 1.9 Hz, 1H), 6.91 (s, 1H), 4.73 (t, J=5.6 Hz, 1H), 3.87 (s, 3H), 3.30-3.21 (m, 2H), 2.96-2.92 (m, 1H), 2.13 (dd, J=13.7, 5.8 Hz, 1H), 1.91-1.83 (m, 1H), 1.51-1.42 (m, 1H), 1.14 (d, J=6.4 Hz, 3H), 1.02-0.98 (m, 2H), 0.93-0.87 (m, 2H).

Step F: N-(5-(4-(((3S,5S)-1,5-dimethylpyrrolidin-3-yl)oxy)-1-methyl-1H-pyrazol-5-yl)pyrazolo[1,5-a]pyridin-2-yl)cyclopropanecarboxamide. To a vial containing N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide (85 mg, 0.22 mmol) was added MeOH (4.47 mL, 0.05 M) and paraformaldehyde (30 mg, 0.34 mmol). The mixture was stirred for 5 min at 40° C., then sodium cyanoborohydride (42 mg, 0.67 mmol) was added. The mixture was stirred for 15 min at 40° C., then extracted with EtOAc/water/brine. The organic layer was separated, dried with anhydrous Na₂SO₄ then purified via FCC (0-20% 2M NH₃ in MeOH/DCM) to give the title compound as a white solid (67 mg, 77%). MS (ESI): mass calcd. for C₂₁H₂₆N₆O₂, 394.2. m/z found, 395.2 [M+H]⁺. ¹H NMR (400 MHz, Methanol-d4) δ 8.48-8.43 (m, 1H), 7.63-7.59 (m, 1H), 7.36 (s, 1H), 6.95-6.90 (m, 2H), 4.68-4.59 (m, 1H), 3.87 (s, 3H), 3.55-3.47 (m, 1H), 2.57-2.44 (m, 1H), 2.41 (dd, J=11.0, 4.4 Hz, 1H), 2.28 (s, 3H), 2.13-2.07 (m, 1H), 1.91-1.83 (m, 1H), 1.78-1.67 (m, 1H), 1.08 (d, J=6.1 Hz, 3H), 1.03-0.97 (m, 2H), 0.94-0.86 (m, 2H).

### Example 2: N-(2,6-dimethylpyrimidin-4-yl)-5-[2-methyl-4-[[(2R)-1-methylazetidin-2-yl]methoxy]pyrazol-3-yl]pyrazolo[1,5-a]pyridin-2-amine

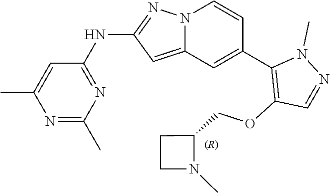

Step A: tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. To a 20 mL microwave vial containing a stir bar was charged Intermediate 6 (250 mg, 0.627 mmol), 4-chloro-2,6-dimethylpyrimidine (98.5 mg, 0.690 mmol), tBuBrettPhos Pd G3 (26.0 mg, 0.0314 mmol), cesium carbonate (613 mg, 1.88 mmol), and 1,4-dioxane (1.3 mL). The reaction mixture was degassed under vacuum with sonication for 2 minutes, then vented to a Ar. The vial was placed into a pre-heated plate at 80° C. for 3 h. The reaction mixture was then filtered, concentrated to dryness under reduced pressure to afford the crude product which was purified by flash column chromatography (eluent: methanol (contain 10% aq. ammonia): DCM=2-10%) to afford the title compound (257 mg, 81%) as a yellow solid.

Step B, (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine hydrate. Trifluoroacetic acid (23 mL) was added to a solution of tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate, Step A, (257 mg, 0.509 mmol) in DCM. The reaction mixture was stirred at room temperature for 1 hour then concentrated to dryness under reduced pressure. The crude product was diluted in MeOH and purified by preparative HPLC with DuraShell 150×25 mm×5 μm, column (eluent: 18% to 48% (v/v) CH₃CN and H₂O with 10 mM NH₄HCO₃) to afford the title compound (200 mg, 97%) as a white solid. MS (ESI): mass calcd. for C₂₁H₂₄N₈O, 404.2. m/z found, 405.2 [M+H]⁺. ¹H NMR (500 MHz, Chloroform-d) δ 8.49-8.43 (m, 1H), 8.15 (s, 1H), 7.48 (t, J=1.9 Hz, 1H), 7.37 (d, J=1.6 Hz, 1H), 7.03 (d, J=3.2 Hz, 1H), 6.85 (dt, J=7.2, 1.8 Hz, 1H), 6.62 (s, 1H), 4.25 (qt, J=6.8, 4.3, 3.5 Hz, 1H), 4.09-3.97 (m, 2H), 3.91 (d, J=1.5 Hz, 3H), 3.71-3.63 (m, 1H), 3.53-3.43 (m, 1H), 3.27 (s, 1H), 2.61 (d, J=1.5 Hz, 3H), 2.44 (d, J=2.2 Hz, 3H), 2.41-2.31 (m, 1H), 2.27-2.16 (m, 1H).

Step C: (R)—N-(2,6-dimethylpyrimidin-4-yl)-5-(1-methyl-4-((1-methylazetidin-2-yl)methoxy)-1H-pyrazol-5-yl)pyrazolo[1,5-a]pyridin-2-amine. H(CHO)n (134 mg, 1.48 mmol) was added to a solution containing (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine, Step B, (400 mg, 0.989 mmol) and MeOH (4.8 mL). The reaction mixture was stirred at room temperature then NaBH₃CN (186 mg, 2.97 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anhydrous MgSO₄, filtered, and concentrated to dryness under reduced pressure to obtain the crude product, which was purified by flash column chromatography (eluent: methanol (contain 10% aq. ammonia): DCM=2-10%) to afford the title compound (101 mg, 24%) as a white solid. MS (ESI): mass calcd. for C₂₂H₂₆N₈O, 418.2. m/z found, 419.2 [M+H]⁺. ¹H NMR (500 MHz, Chloroform-d) δ 8.74 (s, 1H), 8.44 (d, J=7.2 Hz, 1H), 7.47-7.35 (m, 1H), 7.27 (s, 1H), 6.93 (s, 1H), 6.78 (dd, J=7.2, 1.9 Hz, 1H), 6.53 (s, 1H), 3.95-3.88 (m, 3H), 3.83 (s, 3H), 3.42-3.16 (m, 2H), 2.92-2.55 (m, 1H), 2.53 (s, 3H), 2.35 (s, 3H), 2.25 (s, 3H), 1.98-1.82 (m, 1H).

### Example 3: N-[5-[2-methyl-4-[[(2S)-1,3,3-trimethylazetidin-2-yl]methoxy]pyrazol-3-yl]pyrazolo[1,5-a]pyridin-2-yl]cyclopropanecarboxamide

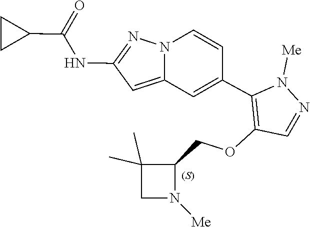

Step A: To a 20 mL vial was charged Intermediate 4 (108 mg, 0.363 mmol), and triphenylphosphine (194 mg, 0.740 mmol) as solids. A solution of tert-butyl (S)-2-(hydroxymethyl)-3,3-dimethylazetidine-1-carboxylate (165 mg, 0.766 mmol) in THE (2.2 mL) was added to the vial. The resulting mixture was stirred at room temperature for 15 min at which point diisopropyl azodicarboxylate (0.140 mL, 0.726 mmol) was added dropwise. The vial was placed into a preheated 60° C. heating block. After 145 min of heating, the reaction was judged complete and cooled to room temperature. The crude reaction solution was poured onto sat. aq. NaHCO₃ then extracted with ethyl acetate (50 mL). The layers were separated, and the aqueous layer was back extracted with ethyl acetate (1×50 mL). The combined organic layers were dried with MgSO₄, filtered, the concentrated to dryness. The crude product was purified by FCC (eluent: dichloromethane:ethyl acetate=1:9 to 1:4) to afford tert-butyl (S)-2-(((5-(2-(cyclopropanecarboxamido)pyrazolo[1,5-a]pyridin-5-yl)-1-methyl-1H-pyrazol-4-yl)oxy)methyl)-3,3-dimethylazetidine-1-carboxylate (165 mg, 92%) as a solid.

Step B: N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide. To a 20 mL vial was charged tert-butyl (S)-2-(((5-(2-(cyclopropanecarboxamido)pyrazolo[1,5-a]pyridin-5-yl)-1-methyl-1H-pyrazol-4-yl)oxy)methyl)-3,3-dimethylazetidine-1-carboxylate (331 mg, 0.669 mmol) as a solid followed by the addition of dichloromethane (4.5 mL) and trifluoroacetic acid (0.51 mL, 6.69 mmol). The resulting reaction solution was stirred at room temperature. At 7 h, the solution was concentrated to dryness. The crude product was purified by FCC (eluent: dichloromethane:2 N NH₃-MeOH=1:0 to 8:1) to the title compound (150 mg, 57%) as a solid.

Step C: N-[5-[2-methyl-4-[[(2S)-1,3,3-trimethylazetidin-2-yl]methoxy]pyrazol-3-yl]pyrazolo[1,5-a]pyridin-2-yl]cyclopropanecarboxamide. The title compound (58 mg, 74%) was prepared as described in Example 2 where N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide was used instead of (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine. MS (ESI): mass calcd. for C₂₂H₂₈N₆O₂, 408.2. m/z found, 409.3 [M+H]⁺. ¹H NMR (400 MHz, DMSO-d6) δ 11.07 (s, 1H), 8.59 (dt, J=7.2, 0.9 Hz, 1H), 7.66 (dd, J=2.0, 1.0 Hz, 1H), 7.47 (s, 1H), 6.93 (dd, J=7.2, 2.0 Hz, 1H), 6.88-6.84 (m, 1H), 4.00-3.88 (m, 2H), 3.85 (s, 3H), 3.00 (d, J=6.0 Hz, 1H), 2.87 (t, J=6.4 Hz, 1H), 2.47 (d, J=6.2 Hz, 1H), 2.20 (s, 3H), 1.98-1.86 (m, 1H), 1.09 (s, 3H), 1.02 (s, 3H), 0.88-0.76 (m, 4H).

### Example 4: N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide

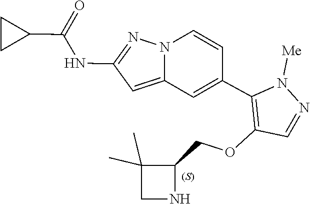

The title compound (150 mg, 57%) was prepared as describe in Example 3, Step B. MS (ESI): mass calcd. for C₂₁H₂₆N₆O₂, 394.2. m/z found, 395.2 [M+H]⁺. ¹H NMR (400 MHz, DMSO-d6) δ 11.08 (s, 1H), 8.59 (dt, J=7.2, 1.0 Hz, 1H), 7.67 (dd, J=2.0, 0.9 Hz, 1H), 7.47 (s, 1H), 6.95 (dd, J=7.2, 2.0 Hz, 1H), 6.88 (d, J=0.9 Hz, 1H), 3.98 (dd, J=9.8, 7.0 Hz, 1H), 3.90 (dd, J=9.8, 6.2 Hz, 1H), 3.65 (t, J=6.6 Hz, 1H), 3.31 (s, 3H), 3.20 (d, J=6.8 Hz, 1H), 2.88 (d, J=6.7 Hz, 1H), 1.98-1.87 (m, 1H), 1.10 (d, J=2.0 Hz, 6H), 0.89-0.77 (m, 4H).

### Example 5: N-(5-(4-(((3*R,4*S)-4-methoxypyrrolidin-3-yl)oxy)-1-methyl-1H-pyrazol-5-yl)pyrazolo[1,5-a]pyridin-2-yl)cyclopropanecarboxamide

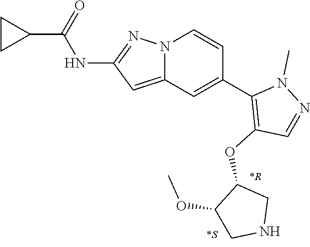

Step A: rac-trans-tert-butyl (3r,4r)-3-methoxy-4-(tosyloxy)pyrrolidine-1-carboxylate. To a 40 mL via was charged trans-rac-tert-butyl (3r,4r)-3-hydroxy-4-methoxypyrrolidine-1-carboxylate (1.0 g, 4.6 mmol) and 4-dimethylaminopyridine (840 mg, 6.9 mmol) followed by dichloromethane (9.2 mL). The vial was placed into an ice bath and p-toluenesulfonyl chloride (1050 mg, 5.5 mmol) was added portionwise. The ice-bath was removed and the reaction stirred at room temperature. At 4 h, silica gel was add ed to the reaction vessel and concentrated to dryness. Purification by FCC (eluent: hexanes:ethyl acetate=1:0 to 8:1) afforded the title compound (1600 mg, 93%) as a solid. ¹H NMR (400 MHz, DMSO-d6) δ 7.90-7.82 (m, 2H), 7.51 (d, J=8.0 Hz, 2H), 4.89 (s, 1H), 3.82 (s, 1H), 3.45-3.35 (m, 2H), 3.28-3.20 (m, 2H), 3.18 (s, 3H), 2.44 (s, 3H), 1.36 (d, J=9.4 Hz, 9H).

Step B: rac-tert-butyl (3r,4s)-3-methoxy-4-((1-methyl-1H-pyrazol-4-yl)oxy)pyrrolidine-1-carboxylate. To a 40 mL vial was charged rac-trans-tert-butyl (3r,4r)-3-methoxy-4-(tosyloxy)pyrrolidine-1-carboxylate (815 mg, 2.19 mmol), 1-methyl-1h-pyrazol-4-ol (269 mg, 2.74 mmol), cesium carbonate (1430 mg, 4.39 mmol), and DMF (4.4 mL). The vial was heated between 80-90° C. for 18 h. The crude reaction mixture was partitioned between ethyl acetate and water/brine. The layers were separated, and the organics were dried over anh. Na₂SO₄, filtered, then concentrated to dryness. The crude product was purified by FCC (eluent: hexanes:ethyl acetate=3:1 to 0:1) to afford the title compound (312 mg, 48%) as a light yellow oil.

Step C: rac-cis-tert-butyl (3s,4r)-3-((5-bromo-1-methyl-1H-pyrazol-4-yl)oxy)-4-methoxypyrrolidine-1-carboxylate. To a 40 mL vial was charged rac-tert-butyl (3r,4s)-3-methoxy-4-((1-methyl-1H-pyrazol-4-yl)oxy)pyrrolidine-1-carboxylate (458 mg, 1.54 mmol) and acetonitrile (7.7 mL). N-bromo succinimide (274 mg, 1.54 mmol) was added portion wise to the vial. The reaction was pushed toward full conversion by the continuous adding of NBS until starting materials were fully consumed. Reaction mixture was diluted with dichloromethane then washed with saturated aq. sodium thiosulfate. The organics were dried with Na₂SO₄ then concentrated to dryness. The crude product was purified by FCC (eluent: hexanes:ethyl acetate=1:0 to 0:1) to afford the titled compound (232 mg, 40%) as a yellow oil.

Step D: rac-cis-tert-butyl (3s,4r)-3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate. To a 20 mL vial was charged rac-cis-tert-butyl (3s,4r)-3-((5-bromo-1-methyl-1H-pyrazol-4-yl)oxy)-4-methoxypyrrolidine-1-carboxylate (215 mg, 0.571 mmol), Intermediate 3 (224 mg, 0.686 mmol), 1,4-dioxane (2.3 mL), and 0.5 M aq. K₃PO₄ (1.1 mL). The vial was evacuated and backfilled with N₂. 1,1′-bis(diphenylphosphino)ferrocene-palladium(ii)dichloride dichloromethane complex (47.1 mg, 0.0571 mmol) was then added. The vial was heated to 90° C. for 18 h. The reaction mixture was partitioned between dichloromethane and water/brine. Layers were separated and the organics were dried with Na₂SO₄, filtered, then concentrated to dryness. The crude product was purified by FCC (eluent: hexanes:ethyl acetate=3:1 to 0:1) to afford the titled compound (265 mg, 94%) of a brown oil.

Step E: N-(5-(4-(((3*R,4*S)-4-methoxypyrrolidin-3-yl)oxy)-1-methyl-1H-pyrazol-5-yl)pyrazolo[1,5-a]pyridin-2-yl)cyclopropanecarboxamide. To a 20 mL vial was charged rac-cis-tert-butyl (3s,4r)-3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate (265 mg, 0.534 mmol), dichloroethane (5.3 mL), followed by dropwise addition of trifluoroacetic acid (0.41 mL, 5.34 mmol). The vial was heated to 70° C. Additional trifluoroacetic acid was required to reach full conversion. The reaction was quenched by addition of sat. aq. NaHCO₃ then extraction using 10% MeOH/DCM.

The organics were dried with anh. Na₂SO₄, filtered, then conc to dryness. The crude product was purified by FCC (eluent: DCM:2 NNH3-MeOH=1:0 to 4:1). The racemic mixture was separated by SFC over IG 2×25 cm, 5 μm (eluent: 60% ethanol (0.1% DEA)/CO₂, 100 bar). The first eluting isomer was designated as (3*R,4*S); the second eluting peak afforded the title compound (19 mg, 9%). MS (ESI): mass calcd. for C₂₀H₂₄N₆O₃, 396.2. m/z found, 397.2 [M+H]⁺. ¹H NMR (500 MHz, Methanol-d4) δ 8.47-8.43 (m, 1H), 7.71-7.67 (m, 1H), 7.46 (s, 1H), 7.02 (dd, J=7.2, 1.9 Hz, 1H), 6.91 (s, 1H), 4.62-4.56 (m, 1H), 3.95-3.90 (m, 1H), 3.89 (s, 3H), 3.33 (s, 3H), 3.18-3.12 (m, 1H), 3.09 (dd, J=11.5, 6.2 Hz, 1H), 3.02 (dd, J=12.3, 4.1 Hz, 1H), 2.92 (dd, J=11.5, 6.3 Hz, 1H), 1.92-1.82 (m, 1H), 1.02-0.97 (m, 2H), 0.92-0.87 (m, 2H).

### Example 6: N-[5-[2-methyl-4-[[(2R)-morpholin-2-yl]methoxy]pyrazol-3-yl]pyrazolo[1,5-a]pyridin-2-yl]cyclopropanecarboxamide

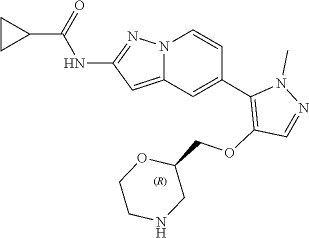

Step A: (R)-tert butyl-2-((tosyloxy)methyl)morpholine-4-carboxylate. The title compound (1.7 g, 99%) was prepared as described in Example 5, step A, using (R)-tert-butyl 2-(hydroxymethyl)morpholine-4-carboxylate in place of trans-rac-tert-butyl (3r,4r)-3-hydroxy-4-methoxypyrrolidine-1-carboxylate.

Step B: tert-butyl (R)-2-(((5-(2-(cyclopropanecarboxamido)pyrazolo[1,5-a]pyridin-5-yl)-1-methyl-1H-pyrazol-4-yl)oxy)methyl)morpholine-4-carboxylate. A mixture consisting of Intermediate 4 (300 mg, 1.01 mmol), (R)-tert-butyl 2-((tosyloxy)methyl)morpholine-4-carboxylate (450 mg, 1.21 mmol), Cs₂CO₃ (986 mg, 3.03 mmol), and DMF (20 mL) was stirred overnight at 35° C. The reaction mixture was concentrated under reduced pressure to give the crude product which was purified by FCC (eluent: petroleum ether:ethyl acetate=1:0 to 1:4) to afford the titled compound (300 mg, 60%) as a light yellow solid.

Step C: N-[5-[2-methyl-4-[[(2R)-morpholin-2-yl]methoxy]pyrazol-3-yl]pyrazolo[1,5-a]pyridin-2-yl]cyclopropanecarboxamide. A solution consisting of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate (150 mg, 0.302 mmol) and HCl/dioxane (3 mL) was stirred at r.t. for 2 h. The reaction mixture was concentrated under reduced pressure to give a crude product which was purified by preparative HPLC with DuraShell 150×25 mm×5 um, column (eluent: 18% to 48% (v/v) CH₃CN and H₂O with 10 mM NH₄HCO₃) to afford product. The product was suspended in water (10 mL), the mixture frozen using dry ice/EtOH, and then lyophilized to dryness to afford the title compound (14.5 mg, 11%) as a white solid. MS (ESI): mass calcd. for C₂₀H₂₄N₆O₃, 396.2. m/z found, 397.2 [M+H]⁺. ¹H NMR (400 MHz, DMSO-d6) δ 11.11 (s, 1H), 8.61 (d, J=7.3 Hz, 1H), 7.70 (d, J=1.2 Hz, 1H), 7.45 (s, 1H), 6.96 (dd, J=1.6, 6.8 Hz, 1H), 6.89 (s, 1H), 3.93-3.86 (m, 2H), 3.84 (s, 3H), 3.74-3.66 (m, 1H), 3.64-3.57 (m, 1H), 3.44-3.38 (m, 1H), 2.82-2.75 (m, 1H), 2.68-2.57 (m, 2H), 2.46-2.40 (m, 1H), 1.97-1.88 (m, 1H), 0.86-0.79 (m, 4H).

### Example 7: 5-[2-methyl-4-[(3S)-1-methylpyrrolidin-3-yl]oxy-pyrazol-3-yl]-N-[6-(trifluoromethyl)pyrazin-2-yl]pyrazolo[1,5-a]pyridin-2-amine

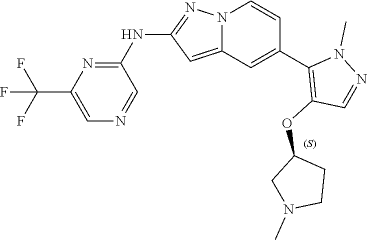

A mixture consisting of (S)-5-(1-methyl-4-((1-methylpyrrolidin-3-yl)oxy)-1H-pyrazol-5-yl)pyrazolo[1,5-a]pyridin-2-amine Intermediate 7 (150 mg, 0.480 mmol), 2-chloro-6-(trifluoromethyl)pyrazine (158 mg, 0.720 mmol), Brettphos-Pd-G3 (87 mg, 0.096 mmol), Brettphos (103 mg, 0.192 mmol), Cs₂CO₃ (469 mg, 1.44 mmol) and 1,4-dioxane (3 mL) was purged with Ar for 2 min and stirred at 50° C. for 6 h. The mixture was filtered through a pad of Celite and the filtrate was concentrated to dryness under reduced pressure to give the crude product, which was purified by FCC (DCM:MeOH=0:100 to 90:10) and purification by preparative HPLC using a Boston Prime C18 150×30 mm×5 μm column (eluent: 40% to 70% (v/v) CH₃CN and H₂O with 0.04% NH₃ and 10 mM NH₄HCO₃) to afford the title compound (86.4 mg, 39%) as a yellow solid. MS (ESI): mass calcd. for C₂₁H₂₁F₃N₈O, 458.2. m/z found, 459.2 [M+H]⁺. ¹H NMR (400 MHz, DMSO-d6) δ 10.95 (br. s., 1H), 8.87 (s, 1H), 8.67 (d, J=7.2 Hz, 1H), 8.47 (s, 1H), 7.77 (s, 1H), 7.40 (s, 1H), 6.98 (dd, J=1.2, 7.2 Hz, 1H), 6.94 (s, 1H), 4.78-4.59 (m, 1H), 3.87 (s, 3H), 2.67-2.58 (m, 3H), 2.33-2.25 (m, 1H), 2.25-2.13 (m, 4H), 1.84-1.71 (m, 1H) 19F NMR (376 MHz, DMSO-d6) δ −67.03 (s, 3F).

### Example 8: N-[5-[2-methyl-4-[(1-methylazetidin-3-yl)methoxy]pyrazol-3-yl]pyrazolo[1,5-a]pyridin-2-yl]cyclopropanecarboxamide

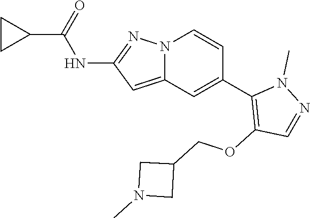 [SMILES: CN1CC(COc2cnn(C)c2-c2ccn3nc(NC(=O)C4CC4)cc3c2)C1]

Step A: N-(5-(4-(azetidin-3-ylmethoxy)-1-methyl-1H-pyrazol-5-yl)pyrazolo[1,5-a]pyridin-2-yl)cyclopropanecarboxamide. The title compound was prepared as described in Example 6 using 1-boc-3-(bromomethyl)azetidine in place of tert-butyl (R)-2-((tosyloxy)methyl)morpholine-4-carboxylate in Step B and trifluoracetic acid in place of HCl in step C.

Step B: N-[5-[2-methyl-4-[(1-methylazetidin-3-yl)methoxy]pyrazol-3-yl]pyrazolo[1,5-a]pyridin-2-yl]cyclopropanecarboxamide. To a solution of N-(5-(4-(azetidin-3-ylmethoxy)-1-methyl-1H-pyrazol-5-yl)pyrazolo[1,5-a]pyridin-2-yl)cyclopropanecarboxamide (1.1 g, 0.60 mmol) in MeOH (5 mL) was added H(CHO)n (81.1 mg, 0.901 mmol) at room temperature. The mixture was stirred for 30 min. Then to the solution was added NaBH₃CN (75.5 mg, 1.20 mmol) and stirred for 30 min. The reaction mixture was directly concentrated and purified by preparative HPLC using Agela ASB, 150×25 mm×5 μm column (eluent: 10% to 40% (v/v) CH₃CN and aqueous HCl (0.006 N) to the title compound (105 mg, 41%). MS (ESI): mass calcd. for C₂₀H₂₄N₆O₂. HCl, 380.2. m/z found, 381.2 [M+H]⁺. ¹H NMR (400 MHz, DMSO-d6) δ 11.24-11.00 (m, 2H), 8.68-8.57 (m, 1H), 7.80-7.71 (m, 1H), 7.56-7.47 (m, 1H), 7.04-6.95 (m, 1H), 6.92 (s, 1H), 4.26-4.12 (m, 2H), 4.05 (d, J=5.2 Hz, 1H), 4.02-3.91 (m, 2H), 3.91-3.84 (m, 3H), 3.83-3.73 (m, 1H), 3.18-3.03 (m, 1H), 2.83-2.63 (m, 3H), 2.03-1.90 (m, 1H), 0.88-0.77 (m, 4H).

### Example 9: N-[5-[2-methyl-4-[[(2S)-1-methylazetidin-2-yl]methoxy]pyrazol-3-yl]pyrazolo[1,5-a]pyridin-2-yl]cyclopropanecarboxamide

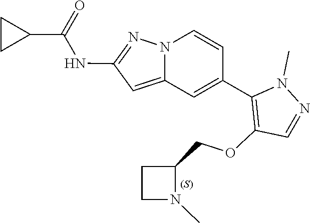

Step A: tert-butyl (S)-2-((tosyloxy)methyl)azetidine-1-carboxylate. A mixture of tert-butyl (S)-2-(hydroxymethyl)azetidine-1-carboxylate (400 mg, 2.14 mmol) and DCM (6 mL) was placed in a 10° C. bath. To the mixture was added TEA (614 mg, 6.07 mmol) and a solution of 4-methylbenzene-1-sulfonyl chloride (611 mg, 3.21 mmol) in DCM (2 mL). N,N-dimethylpyridin-4-amine (26.1 mg, 0.214 mmol) was added, and the mixture was allowed to stir at 25° C. for 4 h.

The reaction mixture was purified by FCC (petroleum ether:ethyl acetate=1:0 to 4:1) to afford the title compound (700 mg, 96% yield) as a colorless oil.

Step B: tert-butyl (S)-2-(((5-(2-(cyclopropanecarboxamido)pyrazolo[1,5-a]pyridin-5-yl)-1-methyl-1H-pyrazol-4-yl)oxy)methyl)azetidine-1-carboxylate. A mixture of Intermediate 4 (300 mg, 1.01 mmol), tert-butyl (S)-2-((tosyloxy)methyl)azetidine-1-carboxylate (413 mg, 1.21 mmol), Cs₂CO₃ (1.64 g, 3.03 mmol) and DMF (10 mL) was stirred at 30° C. for 16 h. The reaction mixture was poured into water (20 mL) and extracted with DCM (10 mL×3). The combined organic extracts were washed with brine (30 mL), dried over anhydrous Na₂SO₄, filtered, and concentrated to dryness under reduced pressure to give the crude product, which was purified by FCC (eluent: petroleum ether:ethyl acetate=1:0 to 0:1) to afford the title compound (350 mg, 74%) as a yellow solid.

Step C: (S)—N-(5-(4-(azetidin-2-ylmethoxy)-1-methyl-1H-pyrazol-5-yl)pyrazolo[1,5-a]pyridin-2-yl)cyclopropanecarboxamide. To tert-butyl (S)-2-(((5-(2-(cyclopropanecarboxamido)pyrazolo[1,5-a]pyridin-5-yl)-1-methyl-1H-pyrazol-4-yl)oxy)methyl)azetidine-1-carboxylate (300 mg, 0.643 mmol) was added DCM/TFA (10:1, 10 mL) and the mixture was stirred at rt for 2 h. The reaction mixture was concentrated to dryness under reduced pressure. The crude product was re-dissolved in water (4 mL) and basified with solid NaHCO₃ to pH=8. The resulting residue was suspended in water (10 mL) and the mixture frozen using dry ice/acetone. Lyophilization afforded the title compound as crude product (100 mg, 42% yield).

Step D: N-[5-[2-methyl-4-[[(2S)-1-methylazetidin-2-yl]methoxy]pyrazol-3-yl]pyrazolo[1,5-a]pyridin-2-yl]cyclopropanecarboxamide. The title compound (50 mg, 73% yield) was prepared as described in Example 8 step B where (S)—N-(5-(4-(azetidin-2-ylmethoxy)-1-methyl-1H-pyrazol-5-yl)pyrazolo[1,5-a]pyridin-2-yl)cyclopropanecarboxamide (65 mg, 0.18 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₀H₂₄N₆O₂, 380.45. m/z found, 381.3 [M+H]⁺. ¹H NMR (400 MHz, CD3OD): 8.44 (d, J=7.2 Hz, 1H), 7.64 (s, 1H), 7.43 (s, 1H), 6.96 (dd, J=2.0, 7.2 Hz, 1H), 6.90 (s, 1H), 4.08-3.94 (m, 2H), 3.88 (s, 3H), 3.52-3.42 (m, 1H), 3.40-3.33 (m, 1H), 2.96-2.85 (m, 1H), 2.31 (s, 3H), 2.11-1.94 (m, 2H), 1.91-1.82 (m, 1H), 1.04-0.96 (m, 2H), 0.94-0.84 (m, 2H).

### Example 10: N-[5-[2-methyl-4-[[(2R,3R)-1-methyl-3-(trifluoromethyl)azetidin-2-yl]methoxy]pyrazol-3-yl]pyrazolo[1,5-a]pyridin-2-yl]cyclopropanecarboxamide

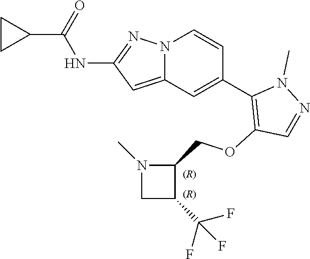

Step A: N-(5-(1-methyl-4-(((2R,3R)-3-(trifluoromethyl)azetidin-2-yl)methoxy)-1H-pyrazol-5-yl)pyrazolo[1,5-a]pyridin-2-yl)cyclopropanecarboxamide. The title compound (75 mg, 2 steps, 41% yield) was prepared as described in Example 3, Steps A-B, where tert-butyl (2R,3R)-2-(hydroxymethyl)-3-(trifluoromethyl)azetidine-1-carboxylate (176 mg, 0.67 mmol) was used instead of tert-butyl (S)-2-(hydroxymethyl)-3,3-dimethylazetidine-1-carboxylate.

Step B: N-[5-[2-methyl-4-[[(2R,3R)-1-methyl-3-(trifluoromethyl)azetidin-2-yl]methoxy]pyrazol-3-yl]pyrazolo[1,5-a]pyridin-2-yl]cyclopropanecarboxamide. The title compound (8.6 mg, 14% yield) was prepared as described in Example 9 then Example 8 step B where N-(5-(1-methyl-4-(((2R,3R)-3-(trifluoromethyl)azetidin-2-yl)methoxy)-1H-pyrazol-5-yl)pyrazolo[1,5-a]pyridin-2-yl)cyclopropanecarboxamide (75 mg, 0.14 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₁H₂₃F₃N₆O₂, 448.447. m/z found, 449.20 40C, 4 h [M+H]⁺. ¹H NMR (500 MHz, Methanol-d4) δ 8.45 (dt, J=7.3, 0.9 Hz, 1H), 7.65 (dd, J=2.0, 1.0 Hz, 1H), 7.44 (s, 1H), 6.96 (dd, J=7.2, 1.9 Hz, 1H), 6.90 (s, 1H), 4.07 (dd, J=10.3, 3.8 Hz, 1H), 4.00 (dd, J=10.3, 6.2 Hz, 1H), 3.88 (s, 3H), 3.54 (t, J=7.9 Hz, 1H), 3.48 (ddd, J=8.0, 6.2, 3.8 Hz, 1H), 3.17 (dq, J=17.0, 8.4 Hz, 1H), 2.95 (t, J=8.1 Hz, 1H), 2.34 (s, 3H), 1.86 (tt, J=8.1, 4.4 Hz, 1H), 0.99 (dt, J=4.5, 3.2 Hz, 2H), 0.90 (dt, J=8.1, 3.3 Hz, 2H).

### Example 11: N-[5-[4-[[(2S,3R)-3-ethyl-1-methyl-azetidin-2-yl]methoxy]-2-methyl-pyrazol-3-yl]pyrazolo[1,5-a]pyridin-2-yl]cyclopropanecarboxamide

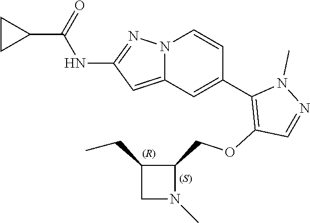

Step A: N-(5-(4-(((2S,3R)-3-ethylazetidin-2-yl)methoxy)-1-methyl-1H-pyrazol-5-yl)pyrazolo[1,5-a]pyridin-2-yl)cyclopropanecarboxamide. The title compound (43 mg, 2 steps, 33%) was prepared as described in Example 3, Steps A-B, where tert-butyl (2S,3R)-3-ethyl-2-(hydroxymethyl)azetidine-1-carboxylate (144 mg, 0.67 mol) was used instead of tert-butyl (S)-2-(hydroxymethyl)-3,3-dimethylazetidine-1-carboxylate.

Step B: N-[5-[4-[[(2S,3R)-3-ethyl-1-methyl-azetidin-2-yl]methoxy]-2-methyl-pyrazol-3-yl]pyrazolo[1,5-a]pyridin-2-yl]cyclopropanecarboxamide. The title compound (21.5 mg, 18% yield) was prepared as described in Example 8 step B where N-(5-(4-(((2S,3R)-3-ethylazetidin-2-yl)methoxy)-1-methyl-1H-pyrazol-5-yl)pyrazolo[1,5-a]pyridin-2-yl)cyclopropanecarboxamide (150 mg, 0.30 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₂H₂₈N₆O₂, 408.2. m/z found, 409.0 [M+H]⁺. ¹H NMR (500 MHz, Methanol-d4) δ 8.44 (dt, J=7.1, 1.0 Hz, 1H), 7.61 (dd, J=1.9, 0.9 Hz, 1H), 7.44 (s, 1H), 6.92 (dd, J=7.2, 1.9 Hz, 1H), 6.89 (s, 1H), 4.10 (dd, J=9.8, 5.3 Hz, 1H), 4.04 (dd, J=9.8, 7.8 Hz, 1H), 3.87 (s, 3H), 3.50 (td, J=7.9, 5.3 Hz, 1H), 3.10 (dd, J=7.7, 2.8 Hz, 1H), 3.01 (t, J=7.8 Hz, 1H), 2.31 (s, 3H), 2.29-2.20 (m, 1H), 1.86 (dp, J=7.8, 4.6 Hz, 1H), 1.72-1.52 (m, 2H), 1.06-0.94 (m, 2H), 0.90 (dt, J=8.0, 3.4 Hz, 2H), 0.82 (t, J=7.3 Hz, 3H).

### Example 12: N-[5-[4-[[(2S,3S)-1,3-dimethylazetidin-2-yl]methoxy]-2-methyl-pyrazol-3-yl]pyrazolo[1,5-a]pyridin-2-yl]cyclopropanecarboxamide

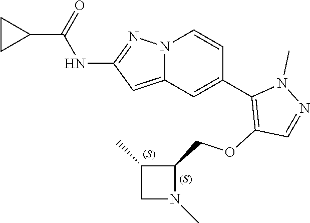

Step A: N-(5-(1-methyl-4-(((2S,3S)-3-methylazetidin-2-yl)methoxy)-1H-pyrazol-5-yl)pyrazolo[1,5-a]pyridin-2-yl)cyclopropanecarboxamide. The title compound (131 mg, 2 steps, 79% yield) was prepared as described in Example 3, Steps A-B, where tert-butyl (2S,3S)-2-(hydroxymethyl)-3-methylazetidine-1-carboxylate was used instead of tert-butyl (S)-2-(hydroxymethyl)-3,3-dimethylazetidine-1-carboxylate.

Step B: N-[5-[4-[[(2S,3S)-1,3-dimethylazetidin-2-yl]methoxy]-2-methyl-pyrazol-3-yl]pyrazolo[1,5-a]pyridin-2-yl]cyclopropanecarboxamide. The title compound (40 mg, 38% yield) was prepared as described in Example 8 step B where N-(5-(1-methyl-4-(((2S,3S)-3-methylazetidin-2-yl)methoxy)-1H-pyrazol-5-yl)pyrazolo[1,5-a]pyridin-2-yl)cyclopropanecarboxamide was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₁H₂₆N₆O₂, 394.5. m/z found, 395.1 [M+H]⁺. ¹H NMR (500 MHz, Methanol-d4) δ 8.44 (dt, J=7.1, 1.0 Hz, 1H), 7.64 (dd, J=2.0, 1.0 Hz, 1H), 7.43 (s, 1H), 6.95 (dd, J=7.2, 1.9 Hz, 1H), 6.89 (s, 1H), 4.05 (dd, J=10.0, 4.1 Hz, 1H), 3.98 (dd, J=10.0, 7.3 Hz, 1H), 3.88 (s, 3H), 3.54 (t, J=7.3 Hz, 1H), 3.03 (td, J=7.7, 4.0 Hz, 1H), 2.53 (dd, J=8.7, 7.1 Hz, 1H), 2.31 (s, 4H), 1.86 (tt, J=8.1, 4.5 Hz, 1H), 1.08 (d, J=6.8 Hz, 3H), 1.02-0.94 (m, 2H), 0.92-0.84 (m, 2H).

### Example 13: (rac)-N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide

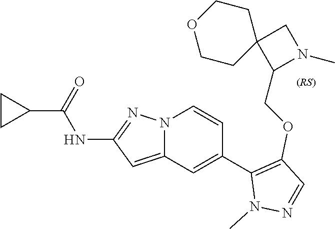 [SMILES: CN1CC2(CCOCC2)C1COc1cnn(C)c1-c1ccn2nc(NC(=O)C3CC3)cc2c1]

Step A: tert-butyl 1-(hydroxymethyl)-7-oxa-2-azaspiro[3.5]nonane-2-carboxylate. To a solution of 2-[(tert-butoxy)carbonyl]-7-oxa-2-azaspiro[3.5]nonane-1-carboxylic acid (1.48 g, 5.54 mmol) in THE (14 mL) at 0° C. was added BH₃-THF (6.4 mL, 1 M in THF, 6.4 mmol) dropwise. The bath was removed, and the resultant solution was stirred at room temperature for 2 h. Then, the solution was quenched with MeOH until bubbling subsided then concentrated to dryness. MeOH was reintroduced and the solution was heated gently to break-up any borane complexes, re-dissolved in DCM, then purified via FCC (0-55% ethyl acetate/DCM, ELSD detection) to afford 1.4 g (72% yield) of the title compound.

Step B: N-(5-(4-((7-oxa-2-azaspiro[3.5]nonan-1-yl)methoxy)-1-methyl-1H-pyrazol-5-yl)pyrazolo[1,5-a]pyridin-2-yl)cyclopropanecarboxamide. The title compound (100 mg, 66% yield) was prepared as described in Example 3, Steps A-B, where tert-butyl 1-(hydroxymethyl)-7-oxa-2-azaspiro[3.5]nonane-2-carboxylate (178 mg, 0.69 mol) was used instead of tert-butyl (S)-2-(hydroxymethyl)-3,3-dimethylazetidine-1-carboxylate.

Step C: N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide. The title compound (67 mg, 70% yield) was prepared as described in Example 8 step B where N-(5-(4-((7-oxa-2-azaspiro[3.5]nonan-1-yl)methoxy)-1-methyl-1H-pyrazol-5-yl)pyrazolo[1,5-a]pyridin-2-yl)cyclopropanecarboxamide (93 mg, 0.21 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₄H₃₀N₆O₃, 450.5. m/z found, 451.3 [M+H]⁺. ¹H NMR (400 MHz, DMSO-d6) δ 11.07 (s, 1H), 8.59 (dt, J=7.3, 0.9 Hz, 1H), 7.66 (dd, J=2.0, 0.9 Hz, 1H), 7.48 (s, 1H), 6.93 (dd, J=7.2, 1.9 Hz, 1H), 6.88-6.83 (m, 1H), 4.03 (dd, J=9.9, 6.6 Hz, 1H), 3.92 (dd, J=9.8, 6.3 Hz, 1H), 3.85 (s, 3H), 3.65 (dt, J=11.4, 3.9 Hz, 1H), 3.57 (dt, J=11.4, 4.0 Hz, 1H), 3.39-3.32 (m, 1H), 3.30-3.26 (m, 1H), 3.19 (td, J=11.1, 2.8 Hz, 1H), 2.91 (t, J=6.4 Hz, 1H), 2.54 (d, J=6.7 Hz, 1H), 2.23 (s, 3H), 1.97-1.85 (m, 1H), 1.70-1.54 (m, 3H), 1.51-1.42 (m, 1H), 0.87-0.77 (m, 4H).

### Example 14: (1R,2R)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide

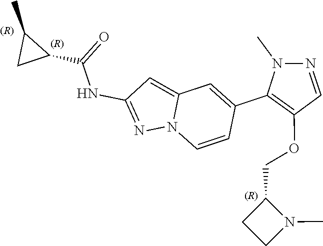

Step A: tert-butyl (R)-2-(((1-methyl-5-(2-((1R,2R)-2-methylcyclopropane-1-carboxamido)pyrazolo[1,5-a]pyridin-5-yl)-1H-pyrazol-4-yl)oxy)methyl)azetidine-1-carboxylate. To a solution of Intermediate 6 (150 mg, 0.376 mmol), (1R, 2R)-2-methylcyclopropane-1-carboxylic acid (113 mg, 1.13 mmol), DIPEA (0.389 mL, 2.26 mmol) in DMF (2.50 mL, 0.15 M) was added (benzotriazole-1-yloxytris(dimethylamino)phosphonium hexafluorophosphate (687 mg, 1.51 mmol) at 23° C. The mixture was stirred for 16 hours and then diluted with EtOAc (5 mL) and 13 wt % NaCl (5 mL). The layers were separated and the organics dried with Na₂SO₄ and filtered. The organics were concentrated and purified by silica gel column chromatography (20-100% EtOAc in hexanes) to provide the titled compound (132 mg, 0.28 mmol, 73%) as a yellow oil which was taken on to the following reaction.

Step B: (1R,2R)—N-(5-(4-(((R)-azetidin-2-yl)methoxy)-1-methyl-1H-pyrazol-5-yl)pyrazolo[1,5-a]pyridin-2-yl)-2-methylcyclopropane-1-carboxamide. To a solution of tert-butyl (R)-2-(((1-methyl-5-(2-((1R,2R)-2-methylcyclopropane-1-carboxamido)pyrazolo[1,5-a]pyridin-5-yl)-1H-pyrazol-4-yl)oxy)methyl)azetidine-1-carboxylate (132 mg, 0.28 mmol) in DCM (2.75 mL, 0.10 M) was added trifluoracetic acid (0.21 mL, 2.75 mmol) and stirred overnight. The solution was purified by silica gel chromatography (0-10% 2M NH₃ in MeOH in DCM) to provide the titled compound (59.6 mg, 0.16 mmol, 57%) as a clear oil which was taken on to the following reaction.

Step C: (1R,2R)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide. To a solution of (1R,2R)—N-(5-(4-(((R)-azetidin-2-yl)methoxy)-1-methyl-1H-pyrazol-5-yl)pyrazolo[1,5-a]pyridin-2-yl)-2-methylcyclopropane-1-carboxamide (59 mg, 0.16 mmol) and paraformaldehyde (21 mg, 0.23 mmol) in MeOH (3.1 mL, 0.05 M) was added sodium cyanoborohydride (29 mg, 0.47 mmol) at 23° C. After 4 hours, the mixture was diluted with EtOAc (5 mL) and water (5 mL).

The layers were separated, and the aqueous layer was extracted with EtOAc (2×5 mL). The organics were combined and dried with Na₂SO₄, filtered and concentrated in vacuo. The residue was purified by silica gel chromatography (0-10% 2M NH₃ in MeOH in DCM) to provide the title compound (26 mg, 0.066 mmol, 43%) as a white solid. MS (ESI): mass calcd. for C₂₁H₂₆N₆O₂, 394.476. m/z found, 395.2 [M+H]⁺. ¹H NMR (400 MHz, Methanol-d4) δ 8.47-8.41 (m, 1H), 7.67-7.62 (m, 1H), 7.43 (s, 1H), 6.96 (dd, J=7.2, 1.9 Hz, 1H), 6.89 (s, 1H), 4.07-3.94 (m, 2H), 3.88 (s, 3H), 3.53-3.43 (m, 1H), 3.41-3.35 (m, 1H), 2.98-2.87 (m, 1H), 2.32 (s, 3H), 2.12-2.02 (m, 1H), 2.01-1.93 (m, 1H), 1.64-1.55 (m, 1H), 1.46-1.34 (m, 1H), 1.23-1.19 (m, 1H), 1.17 (d, J=6.0 Hz, 3H), 0.77-0.70 (m, 1H).

### Example 15: (1S,2S)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide

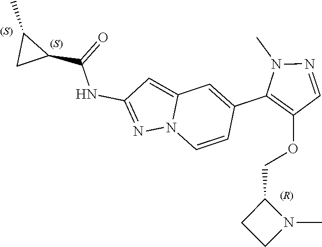

To a vial containing Intermediate 9 (100 mg, 0.32 mmol), (1S, 2S)-2-methylcyclopropane-1-carboxylic acid (96.2 mg, 0.96 mmol) and DIPEA (0.33 mL, 1.92 mmol) in DMF (2.10 mL, 0.15 M) was added (benzotriazole-1-yloxytris(dimethylamino)phosphonium hexafluorophosphate (584 mg, 1.28 mmol) at 23° C. The mixture was stirred for 16 hours and then diluted with water (20 mL) and extracted with DCM (3×30 mL). The combined organics were dried with anh. MgSO₄, filtered and then concentrated in vacuo. The residue was purified by reverse phase basic HPLC using 10-100% MeCN in water (20 mM NH₄OH) to give the title compound (64 mg, 0.16 mmol, 51%) as a white solid. MS (ESI): mass calcd. for C₂₁H₂₆N₆O₂, 394.476. m/z found, 395.20 [M+H]⁺. ¹H NMR (500 MHz, Chloroform-d) δ 9.19-9.12 (m, 1H), 8.23 (d, J=7.2 Hz, 1H), 7.42 (dd, J=1.9, 0.9 Hz, 1H), 7.26 (s, 1H), 6.94 (s, 1H), 6.77 (dd, J=7.2, 1.9 Hz, 1H), 3.93-3.84 (m, 2H), 3.83 (s, 3H), 3.33 (td, J=7.3, 2.9 Hz, 1H), 3.27-3.20 (m, 1H), 2.76-2.70 (m, 1H), 2.24 (s, 3H), 1.97-1.84 (m, 2H), 1.48-1.41 m, 1H), 1.28-1.18 (m, 2H), 1.07-1.02 (m, 3H), 0.62 (td, J=7.1, 3.8 Hz, 1H).

### Example 16: (*R)—N-(5-(4-((5-Ethyl-5-azaspiro[2.3]hexan-4-yl)methoxy)-1-methyl-1H-pyrazol-5-yl)pyrazolo[1,5-a]pyridin-2-yl)cyclopropanecarboxamide

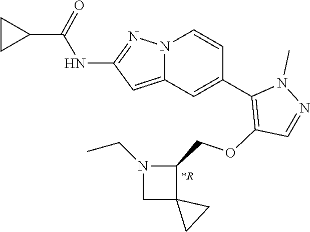 [SMILES: CCN1CC2(CC2)[C@@H]1COc1cnn(C)c1-c1ccn2nc(NC(=O)C3CC3)cc2c1]

Step A: (rac)-tert-Butyl 4-(((5-(2-(cyclopropanecarboxamido)pyrazolo[1,5-a]pyridin-5-yl)-1-methyl-1H-pyrazol-4-yl)oxy)methyl)-5-azaspiro[2.3]hexane-5-carboxylate. To a 20 mL vial was charged N-(5-(4-hydroxy-1-methyl-1H-pyrazol-5-yl)pyrazolo[1,5-a]pyridin-2-yl)cyclopropanecarboxamide (258 mg, 0.87 mmol), and triphenylphosphine (466 mg, 1.78 mmol) as solids. A solution of tert-butyl 4-(hydroxymethyl)-5-azaspiro[2.3]hexane-5-carboxylate (371 mg, 1.74 mmol) in THE (4.5 mL) was added to the vial. The resulting mixture was stirred at room temperature for 15 min at which point diisopropyl azodicarboxylate (0.35 mL, 1.78 mmol) was added dropwise. The vial was placed into a preheated 60° C. heating block. After 3 hours of heating, the reaction was judged complete and cooled to room temperature. The crude reaction solution was poured onto aqueous saturated NaHCO₃ then extracted with ethyl acetate (50 mL). The layers were separated, and the aqueous layer was back extracted with ethyl acetate (1×50 mL). The combined organic layers were dried with anh. MgSO₄, filtered, the concentrated to dryness. The crude product was purified by FCC (eluent: dichloromethane:ethyl acetate=1:9 to 1:4) to afford (rac)-tert-Butyl 4-(((5-(2-(cyclopropanecarboxamido)pyrazolo[1,5-a]pyridin-5-yl)-1-methyl-1H-pyrazol-4-yl)oxy)methyl)-5-azaspiro[2.3]hexane-5-carboxylate (880 mg, 206%) as a solid.

Step B: N-(5-(4-((5-Azaspiro[2.3]hexan-4-yl)methoxy)-1-methyl-1H-pyrazol-5-yl)pyrazolo[1,5-a]pyridin-2-yl)cyclopropanecarboxamide. To a 20 mL vial was charged (R, S)-tert-butyl 4-(((5-(2-(cyclopropanecarboxamido)pyrazolo[1,5-a]pyridin-5-yl)-1-methyl-1H-pyrazol-4-yl)oxy)methyl)-5-azaspiro[2.3]hexane-5-carboxylate (427 mg, 0.87 mmol) as a solid followed by the addition of dichloromethane (5.4 mL) and trifluoroacetic acid (1.1 mL). The resulting reaction solution was stirred at room temperature. At 2 h, the solution was concentrated to dryness. The crude product was purified by FCC (eluent: dichloromethane:2 N NH₃-MeOH=1:0 to 8:1) to obtain the title compound (246 mg, 69%) as a solid. MS (ESI): mass calcd. for C₂₁H₂₄N₆O₂, 392.2. m/z found, 393.2 [M+H]⁺. ¹H NMR (400 MHz, DMSO-d6) δ 11.07 (s, 1H), 8.58 (dt, J=7.2, 1.0 Hz, 1H), 7.66 (dd, J=2.0, 0.9 Hz, 1H), 7.46 (s, 1H), 6.93 (dd, J=7.2, 2.0 Hz, 1H), 6.88 (s, 1H), 4.03 (t, J=5.8 Hz, 1H), 3.98-3.88 (m, 2H), 3.85 (s, 3H), 3.59 (d, J=7.4 Hz, 1H), 3.34 (d, J=7.4 Hz, 1H), 1.93 (m, J=7.7, 3.7 Hz, 1H), 0.88-0.75 (m, 4H), 0.68-0.61 (m, 1H), 0.51-0.44 (m, 1H), 0.42-0.36 (m, 1H), 0.32-0.25 (m, 1H).

Step C:(*R)—N-(5-(4-((5-Azaspiro[2.3]hexan-4-yl)methoxy)-1-methyl-1H-pyrazol-5-yl)pyrazolo[1,5-a]pyridin-2-yl)cyclopropanecarboxamide. The mixture of N-(5-(4-((5-azaspiro[2.3]hexan-4-yl)methoxy)-1-methyl-1H-pyrazol-5-yl)pyrazolo[1,5-a]pyridin-2-yl)cyclopropanecarboxamide isomers (236 mg) was separated by chiral SFC (stationary phase: Daicel Chiralpak AD-H 5 μm 250×20 mm, 30-45% ethanol (0.1% NPA)/CO₂) to afford two diastereomers. The first eluting isomer was assigned as (*R) isomer (86 mg). MS (ESI): mass calcd. for C₂₁H₂₄N₆O₂, 392.2. m/z found, 393.2 [M+H]⁺. ¹H NMR (500 MHz, DMSO-d6) δ 11.07 (s, 1H), 8.58 (dd, J=7.2, 1.0 Hz, 1H), 7.65 (dd, J=2.0, 0.9 Hz, 1H), 7.45 (s, 1H), 6.92 (dd, J=7.2, 2.0 Hz, 1H), 6.87 (s, 1H), 4.01-3.86 (m, 3H), 3.84 (s, 3H), 3.56 (d, J=7.2 Hz, 1H), 1.99-1.86 (m, 1H), 0.90-0.76 (m, 4H), 0.68-0.60 (m, 1H), 0.50-0.42 (m, 1H), 0.41-0.33 (m, 1H), 0.30-0.23 (m, 1H). The second eluting isomer was assigned as (*S) isomer (86 mg). MS (ESI): mass calcd. for C₂₁H₂₄N₆O₂, 392.2. m/z found, 393.2 [M+H]⁺. ¹H NMR (500 MHz, DMSO-d6) δ 10.83 (s, 1H), 8.38-8.27 (m, 1H), 7.42 (dd, J=2.0, 0.9 Hz, 1H), 7.21 (s, 1H), 6.69 (dd, J=7.2, 2.0 Hz, 1H), 6.63 (s, 1H), 3.79 (t, J=5.8 Hz, 1H), 3.74-3.65 (m, 2H), 3.61 (s, 3H), 3.35 (d, J=7.4 Hz, 1H), 1.74-1.63 (m, 1H), 0.58 (tt, J=8.0, 3.0 Hz, 4H), 0.44-0.37 (m, 1H), 0.27-0.20 (m, 1H), 0.19-0.12 (m, 1H), 0.08-−0.02 (m, 1H).

Step D: (*R)—N-(5-(4-((5-Ethyl-5-azaspiro[2.3]hexan-4-yl)methoxy)-1-methyl-1H-pyrazol-5-yl)pyrazolo[1,5-a]pyridin-2-yl)cyclopropanecarboxamide. To a 10 mL microwave vial was charged (*R)—N-(5-(4-((5-azaspiro[2.3]hexan-4-yl)methoxy)-1-methyl-1H-pyrazol-5-yl)pyrazolo[1,5-a]pyridin-2-yl)cyclopropanecarboxamide (30 mg, 0.08 mmol) as a solid followed by addition of a solution of iodoethane (14 mg, 0.09 mmol) in ACN (0.3 mL, 0.3 M) and solid cesium carbonate (74.7 mg, 0.23 mmol) was added. The vial was capped then placed into a preheated 70° C. heating block and stirred vigorously for 2 hour. The reaction mixture was quenched with ethyl acetate (40 mL), filtered through a pad of celite and the resulting filtrate was concentrated to dryness. The residue was purified by flash column chromatography (eluent: methanol (contain 10% aq. ammonia): DCM=2-10%) followed by reverse phase HPLC (Xbridge C18, 5 μM, 50×100; eluent=10-80% 20 mM NH₄OH in water, ACN) to afford the title compound (15 mg, 46%) as a white solid. MS (ESI): mass calcd. for C₂₃H₂₈N₆O₂, 420.2. m/z found, 421.3 [M+H]⁺. ¹H NMR (500 MHz, Chloroform-d) δ 11.07 (s, 1H), 8.58 (dd, J=7.2, 1.0 Hz, 1H), 7.62 (dd, J=2.0, 0.9 Hz, 1H), 7.45 (s, 1H), 6.89 (dd, J=7.2, 2.0 Hz, 1H), 6.87-6.84 (m, 1H), 3.94 (dd, J=9.6, 5.9 Hz, 1H), 3.84 (s, 3H), 3.81 (dd, J=9.7, 6.4 Hz, 1H), 3.42 (t, J=6.1 Hz, 1H), 3.25 (dd, J=6.9, 0.8 Hz, 1H), 3.00 (d, J=6.9 Hz, 1H), 2.63 (dq, J=11.3, 7.3 Hz, 1H), 2.40 (dq, J=11.3, 7.1 Hz, 1H), 1.93 (tt, J=7.7, 3.7 Hz, 1H), 0.89-0.78 (m, 7H), 0.67-0.59 (m, 1H), 0.45-0.38 (m, 1H), 0.36-0.28 (m, 2H).

### Example 17: N-(5,6-Dimethylpyridazin-3-yl)-5-[2-methyl-4-[[(2R)-1-methylazetidin-2-yl]methoxy]pyrazol-3-yl]pyrazolo[1,5-a]pyridin-2-amine

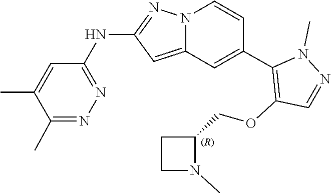

Step A: tert-Butyl (R)-2-(((5-(2-((5,6-dimethylpyridazin-3-yl)amino)pyrazolo[1,5-a]pyridin-5-yl)-1-methyl-1H-pyrazol-4-yl)oxy)methyl)azetidine-1-carboxylate. To a 20 mL microwave vial containing a stir bar was charged Intermediate 6 (500 mg, 0.627 mmol), 6-chloro-3,4-dimethylpyridazine (197 mg, 1.38 mmol), tBuBrettPhos Pd G3 (54 mg, 0.06 mmol), cesium carbonate (1.23 g, 3.76 mmol), and 1,4-dioxane (10 mL). The reaction mixture was degassed under vacuum with sonication for 2 minutes, then vented to argon. The vial was placed into a pre-heated plate at 80° C. for 3 h. The reaction mixture was then filtered, and concentrated to dryness under reduced pressure to afford the crude product which was purified by flash column chromatography (eluent: methanol (contain 10% aq. ammonia): DCM=2-10%) to afford the title compound (420 mg, 66%) as a yellow solid. MS (ESI): mass calcd. for C₂₆H₃₂N₈O₃, 504.3. m/z found, 505.3 [M+H]⁺.

Step B: (R)-5-(4-(Azetidin-2-ylmethoxy)-1-methyl-1H-pyrazol-5-yl)-N-(5,6-dimethylpyridazin-3-yl)pyrazolo[1,5-a]pyridin-2-amine. Trifluoroacetic acid (10 mL) was added to a solution of tert-butyl (R)-2-(((5-(2-((5,6-dimethylpyridazin-3-yl)amino)pyrazolo[1,5-a]pyridin-5-yl)-1-methyl-1H-pyrazol-4-yl)oxy)methyl)azetidine-1-carboxylate (420 mg, 0.83 mmol) in DCM (20 mL). The reaction mixture was stirred at room temperature for 1 hour then concentrated to dryness under reduced pressure. The crude product was diluted in MeOH and purified by preparative HPLC with DuraShell 150×25 mm×5 μm, column (eluent: 10% to 100% (v/v) CH₃CN and H₂O with 10 mM NH₄OH) to afford the title compound (310 mg, 92%) as a white solid. MS (ESI): mass calcd. for C₂₁H₂₄N₈O, 404.2. m/z found, 405.2 [M+H]⁺. ¹H NMR (500 MHz, Chloroform-d) δ 8.95 (s, 1H), 8.33-8.27 (m, 1H), 7.67 (s, 1H), 7.31 (dd, J=2.0, 0.9 Hz, 1H), 7.27 (s, 1H), 6.70 (dd, J=7.2, 1.9 Hz, 1H), 6.56 (s, 1H), 5.22 (s, 1H), 4.24-4.17 (m, 1H), 4.00 (dd, J=10.0, 6.6 Hz, 1H), 3.94 (dd, J=10.0, 4.4 Hz, 1H), 3.82 (s, 3H), 3.62 (q, J=8.1 Hz, 1H), 3.46-3.41 (m, 1H), 3.01 (s, 3H), 2.48 (s, 3H), 2.35-2.10 (m, 2H).

Step C: (R)—N-(5,6-Dimethylpyridazin-3-yl)-5-(1-methyl-4-((1-methylazetidin-2-yl)methoxy)-1H-pyrazol-5-yl)pyrazolo[1,5-a]pyridin-2-amine. H(CHO)n (26.7 mg, 0.30 mmol) was added to a solution containing (R)-5-(4-(azetidin-2-ylmethoxy)-1-methyl-1H-pyrazol-5-yl)-N-(5,6-dimethylpyridazin-3-yl)pyrazolo[1,5-a]pyridin-2-amine (120 mg, 0.30 mmol) and MeOH (10 mL). The reaction mixture was stirred at room temperature then NaBH₃CN (33.6 mg, 0.53 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anh. MgSO₄, filtered, and concentrated to dryness under reduced pressure to obtain the crude product, which was purified by flash column chromatography (eluent: methanol (contain 10% aq. ammonia): DCM=2-10% to afford the title compound (81 mg, 65%) as a white solid. MS (ESI): mass calcd. for C₂₂H₂₆N₈O, 418.2. m/z found, 419.2 [M+H]⁺. ¹H NMR (400 MHz, Chloroform-d) δ 9.96 (s, 1H), 8.47-8.40 (m, 1H), 7.86 (s, 1H), 7.45 (dd, J=1.9, 0.9 Hz, 1H), 7.34 (s, 1H), 6.82-6.74 (m, 2H), 3.96 (d, J=5.7 Hz, 2H), 3.90 (s, 3H), 3.48-3.35 (m, 1H), 3.35-3.27 (m, 1H), 2.84-2.76 (m, 1H), 2.59 (s, 3H), 2.53 (s, 1H), 2.32 (s, 3H), 2.24 (d, J=0.9 Hz, 2H), 2.06-1.89 (m, 2H).

### Example 18: (R)—N-(5,6-Dimethylpyridazin-3-yl)-5-(4-((1-ethylazetidin-2-yl)methoxy)-1-methyl-1H-pyrazol-5-yl)pyrazolo[1,5-a]pyridin-2-amine

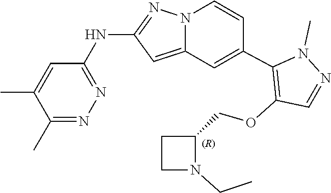

Acetaldehyde (7.5 mg, 0.15 mmol) was added to a solution containing (R)-5-(4-(azetidin-2-ylmethoxy)-1-methyl-1H-pyrazol-5-yl)-N-(5,6-dimethylpyridazin-3-yl)pyrazolo[1,5-a]pyridin-2-amine (80 mg, 0.15 mmol) and MeOH (5 mL). The reaction mixture was stirred at room temperature then NaBH₃CN (21.3 mg, 0.34 mmol) was added to the reaction mixture. The resultant mixture was stirred at room temperature for 60 minutes. The reaction mixture was quenched with water (50 mL) and extracted with 10% MeOH/DCM (50 mL×2). The organic extracts were dried over anhydrous MgSO₄, filtered, and concentrated to dryness under reduced pressure to obtain the crude product, which was purified by flash column chromatography (eluent: methanol (contain 10% aq. ammonia): DCM=2-10%) to afford the title compound (39 mg, 58%) as a white solid. MS (ESI): mass calcd. for C₂₃H₂₈N₈O, 432.2. m/z found, 433.3 [M+H]⁺. ¹H NMR (400 MHz, Chloroform-d) δ 9.51 (s, 1H), 8.35 (dt, J=7.2, 0.9 Hz, 1H), 7.76 (s, 1H), 7.36 (dd, J=2.0, 1.0 Hz, 1H), 7.26 (s, 1H), 6.76-6.63 (m, 2H), 3.98-3.79 (m, 2H), 3.83 (s, 3H), 3.38-3.23 (m, 2H), 3.15 (d, J=4.9 Hz, 1H), 2.76-2.53 (m, 2H), 2.52 (s, 3H), 2.35-2.21 (m, 1H), 2.25 (s, 2H), 2.02-1.83 (m, 2H), 0.87 (t, J=7.2 Hz, 3H).

### Example 19: 5-[4-[[(2R)-1-Ethylazetidin-2-yl]methoxy]-2-methyl-pyrazol-3-yl]-N-(6-methylpyrimidin-4-yl)pyrazolo[1,5-a]pyridin-2-amine

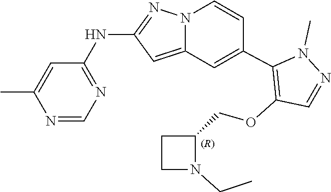

Acetaldehyde (19 mg, 0.42 mmol) was added to a solution of (R)-5-(4-(azetidin-2-ylmethoxy)-1-methyl-1H-pyrazol-5-yl)-N-(6-methylpyrimidin-4-yl)pyrazolo[1,5-a]pyridin-2-amine (150 mg, 0.38 mmol) and MeOH (10 mL). The reaction mixture was stirred at room temperature then NaBH₃CN (21.3 mg, 0.34 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anh. MgSO₄, filtered, and concentrated to dryness under reduced pressure to obtain the crude product. The crude was diluted in MeOH and purified by preparative HPLC with DuraShell 150×25 mm×5 μm, column (eluent: 10% to 100% (v/v) CH₃CN and H₂O with 10 mM NH₄OH) to afford the title compound (122 mg, 76%) as a white solid. MS (ESI): mass calcd. for C₂₂H₂₆N₈O, 418.2. m/z found, 419.2 [M+H]⁺. ¹H NMR (500 MHz, Chloroform-d) δ 9.45 (s, 1H), 8.69 (d, J=1.2 Hz, 1H), 8.38 (dt, J=7.2, 0.9 Hz, 1H), 7.39 (dd, J=2.0, 0.9 Hz, 1H), 7.27 (s, 1H), 7.20 (s, 1H), 6.78 (dd, J=7.2, 1.9 Hz, 1H), 6.51 (s, 1H), 3.94 (dd, J=9.8, 6.4 Hz, 1H), 3.88 (dd, J=9.8, 4.8 Hz, 1H), 3.83 (s, 3H), 3.40-3.24 (m, 2H), 2.72-2.65 (m, 1H), 2.62-2.54 (m, 1H), 2.39 (s, 3H), 2.31-2.22 m, 1H), 2.00-1.83 (m, 2H), 0.86 (t, J=7.2 Hz, 3H).

### Example 20: (R)-5-(4-((1-Ethylazetidin-2-yl)methoxy)-1-methyl-1H-pyrazol-5-yl)-N-(6-methylpyrazin-2-yl)pyrazolo[1,5-a]pyridin-2-amine

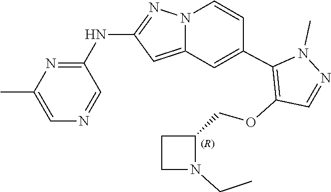

Acetaldehyde (25 mg, 0.56 mmol) was added to a solution of (R)-5-(4-(azetidin-2-ylmethoxy)-1-methyl-1H-pyrazol-5-yl)-N-(6-methylpyrazin-2-yl)pyrazolo[1,5-a]pyridin-2-amine (200 mg, 0.51 mmol) and MeOH (10 mL). The reaction mixture was stirred at room temperature then NaBH₃CN (21.3 mg, 0.34 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anh. MgSO₄, filtered, and concentrated to dryness under reduced pressure to obtain the crude product. The crude was diluted in MeOH and purified by preparative HPLC with DuraShell 150×25 mm×5 μm, column (eluent: 10% to 100% (v/v) CH₃CN and H₂O with 10 mM NH₄OH) to afford the title compound (173 mg, 81%) as a white solid. MS (ESI): mass calcd. for C₂₂H₂₆N₈O, 418.2. m/z found, 419.2 [M+H]⁺. ¹H NMR (400 MHz, Chloroform-d) δ 8.84-8.77 (m, 1H), 8.45-8.37 (m, 2H), 7.92 (s, 1H), 7.44 (dd, J=1.9, 0.9 Hz, 1H), 7.32 (s, 1H), 6.82-6.75 (m, 2H), 4.04-3.88 (m, 2H), 3.88 (s, 3H), 3.45-3.28 (m, 2H), 2.78-2.58 (m, 2H), 2.46 (s, 3H), 2.31 (dq, J=11.4, 7.1 Hz, 1H), 2.06-1.87 (m, 2H), 0.91 (t, J=7.2 Hz, 3H).

### Example 21: (R)-5-(4-((1-Ethylazetidin-2-yl)methoxy)-1-methyl-1H-pyrazol-5-yl)-N-(5-methylpyrazin-2-yl)pyrazolo[1,5-a]pyridin-2-amine

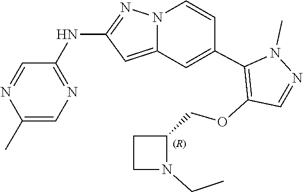

Acetaldehyde (19 mg, 0.42 mmol) was added to a solution of (R)-5-(4-(azetidin-2-ylmethoxy)-1-methyl-1H-pyrazol-5-yl)-N-(5-methylpyrazin-2-yl)pyrazolo[1,5-a]pyridin-2-amine (150 mg, 0.38 mmol) and MeOH (10 mL). The reaction mixture was stirred at room temperature then NaBH₃CN (53.1 mg, 0.85 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anhydrous MgSO₄, filtered, and concentrated to dryness under reduced pressure to obtain the crude product. The crude product was diluted in MeOH and purified by preparative HPLC with DuraShell 150×25 mm×5 μm, column (eluent: 10% to 100% (v/v) CH₃CN and H₂O with 10 mM NH₄OH) to afford the title compound (115 mg, 72%) as a white solid. MS (ESI): mass calcd. for C₂₂H₂₆N₈O, 418.2. m/z found, 419.2 [M+H]⁺. ¹H NMR (400 MHz, Chloroform-d) δ 8.58 (dd, J=20.6, 2.0 Hz, 2H), 8.39 (dt, J=7.2, 0.9 Hz, 1H), 8.11-8.06 (m, 1H), 7.44 (dd, J=1.9, 0.9 Hz, 1H), 7.34 (s, 1H), 6.80 (dd, J=7.2, 1.9 Hz, 1H), 6.69 (d, J=0.8 Hz, 1H), 4.05-3.90 (m, 2H), 3.89 (s, 3H), 3.43-3.30 (m, 2H), 2.80-2.59 (m, 2H), 2.47 (s, 3H), 2.39-2.28 (m, 1H), 2.09-1.89 (m, 2H), 0.93 (t, J=7.2 Hz, 3H).

### Example 22: (R)—N-(5,6-Dimethylpyrazin-2-yl)-5-(4-((1-ethylazetidin-2-yl)methoxy)-1-methyl-1H-pyrazol-5-yl)pyrazolo[1,5-a]pyridin-2-amine

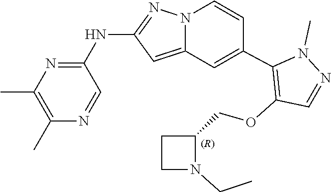

Step A: tert-butyl (R)-2-(((5-(2-((5,6-dimethylpyrazin-2-yl)amino)pyrazolo[1,5-a]pyridin-5-yl)-1-methyl-1H-pyrazol-4-yl)oxy)methyl)azetidine-1-carboxylate. To a 20 mL microwave vial containing a stir bar was charged Intermediate 6 (400 mg, 1.00 mmol), 5-bromo-2,3-dimethylpyrazine (207 mg, 1.10 mmol), tBuBrettPhos Pd G3 (43 mg, 0.05 mmol), cesium carbonate (981 mg, 3.01 mmol), and 1,4-dioxane (10 mL). The reaction mixture was degassed under vacuum with sonication for 2 minutes, then vented to argon. The vial was placed into a pre-heated plate at 80° C. for 3 h. The reaction mixture was then filtered, concentrated to dryness under reduced pressure to afford the crude product which was purified by flash column chromatography (eluent: methanol (contain 10% aq. ammonia): DCM=2-10%) to afford the title compound (450 mg, 89%) as a yellow solid. MS (ESI): mass calcd. for C₂₆H₃₂N₈O₃, 504.3. m/z found, 505.3 [M+H]⁺.

Step B: (R)-5-(4-(Azetidin-2-ylmethoxy)-1-methyl-1H-pyrazol-5-yl)-N-(5,6-dimethylpyrazin-2-yl)pyrazolo[1,5-a]pyridin-2-amine. Trifluoroacetic acid (5 mL) was added to a solution of tert-butyl (R)-2-(((5-(2-((5,6-dimethylpyridazin-3-yl)amino)pyrazolo[1,5-a]pyridin-5-yl)-1-methyl-1H-pyrazol-4-yl)oxy)methyl)azetidine-1-carboxylate (450 mg, 0.89 mmol) in DCM (10 mL). The reaction mixture was stirred at room temperature for 1 hour then concentrated to dryness under reduced pressure. The crude product was diluted in MeOH and purified by preparative HPLC with DuraShell 150×25 mm×5 μm, column (eluent: 10% to 100% (v/v) CH₃CN and H₂O with 10 mM NH₄OH) to afford the title compound (320 mg, 89%) as a white solid. MS (ESI): mass calcd. for C₂₁H₂₄N₈O, 404.2. m/z found, 405.2 [M+H]⁺.

Step C: (R)—N-(5,6-Dimethylpyrazin-2-yl)-5-(4-((1-ethylazetidin-2-yl)methoxy)-1-methyl-1H-pyrazol-5-yl)pyrazolo[1,5-a]pyridin-2-amine. Acetaldehyde (14.4 mg, 0.33 mmol) was added to a solution containing (R)-5-(4-(azetidin-2-ylmethoxy)-1-methyl-1H-pyrazol-5-yl)-N-(5,6-dimethylpyrazin-2-yl)pyrazolo[1,5-a]pyridin-2-amine (120 mg, 0.30 mmol) and MeOH (10 mL). The reaction mixture was stirred at room temperature then NaBH₃CN (33.6 mg, 0.53 mmol) was added to the reaction mixture. The resultant mixture was stirred at room temperature for 60 minutes. The reactant mixture was quenched with water (50 mL) and extracted with 10% MeOH/DCM (50 mL×2). The organic extracts were dried over anhydrous MgSO₄, filtered, and concentrated to dryness under reduced pressure to obtain the crude product, which was purified by flash column chromatography (eluent: methanol (contain 10% aq. ammonia): DCM=2-10%) to afford the title compound (69 mg, 54%) as a white solid. MS (ESI): mass calcd. for C₂₃H₂₈N₈O, 432.2. m/z found, 433.3 [M+H]⁺. ¹H NMR (400 MHz, Chloroform-d) δ 8.46-8.36 (m, 2H), 7.96 (s, 1H), 7.45 (dd, J=1.9, 0.9 Hz, 1H), 7.35 (s, 1H), 6.80 (dd, J=7.2, 1.9 Hz, 1H), 6.69 (d, J=0.8 Hz, 1H), 4.06-3.91 (m, 2H), 3.91 (s, 3H), 3.46-3.31 (m, 2H), 2.81-2.61 (m, 2H), 2.48 (d, J=7.3 Hz, 6H), 2.36 (dt, J=11.4, 7.2 Hz, 1H), 2.10-1.98 (m, 1H), 2.03-1.90 (m, 1H), 0.95 (t, J=7.2 Hz, 3H).

### Example 23: N-(5,6-Dimethylpyrazin-2-yl)-5-[2-methyl-4-[[(2R)-1-methylazetidin-2-yl]methoxy]pyrazol-3-yl]pyrazolo[1,5-a]pyridin-2-amine

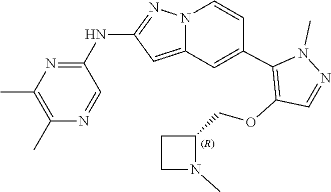

H(CHO)n (66.8 mg, 0.74 mmol) was added to a solution containing (R)-5-(4-(azetidin-2-ylmethoxy)-1-methyl-1H-pyrazol-5-yl)-N-(5,6-dimethylpyrazin-2-yl)pyrazolo[1,5-a]pyridin-2-amine (200 mg, 0.49 mmol) and MeOH (10 mL). The reaction mixture was stirred at room temperature then NaBH₃CN (93.2 mg, 1.48 mmol) was added to the reaction mixture. The resultant mixture was stirred at room temperature for 60 minutes. The reaction mixture was quenched with water (50 mL) and extracted with 10% MeOH/DCM (50 mL×2). The organic extracts were dried over anhydrous MgSO₄, filtered, and concentrated to dryness under reduced pressure to obtain the crude product, which was purified by flash column chromatography (eluent: methanol (containing 10% aq. ammonia): DCM=2-10%) to afford the title compound (147 mg, 71%) as a white solid. MS (ESI): mass calcd. for C₂₂H₂₆N₈O, 418.2. m/z found, 419.2 [M+H]⁺. ¹H NMR (500 MHz, Chloroform-d) δ 8.51 (t, J=2.2 Hz, 1H), 8.44-8.36 (m, 2H), 7.42 (dd, J=1.9, 0.9 Hz, 1H), 7.32 (s, 1H), 6.78 (dd, J=7.1, 1.9 Hz, 1H), 6.69 (d, J=0.8 Hz, 1H), 4.00-3.90 (m, 2H), 3.88 (s, 3H), 3.44-3.35 (m, 1H), 3.33-3.27 (m, 1H), 2.81-2.75 (m, 1H), 2.46 (s, 3H), 2.43 (s, 3H), 2.30 (s, 3H), 2.02-1.89 (m, 2H).

### Example 24: N-[5-[4-[[(2R,3R)-1,3-Dimethylazetidin-2-yl]methoxy]-2-methyl-pyrazol-3-yl]pyrazolo[1,5-a]pyridin-2-yl]cyclopropanecarboxamide

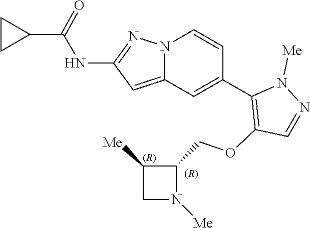

Step A: N-(5-(1-Methyl-4-(((2R,3R)-3-methylazetidin-2-yl)methoxy)-1H-pyrazol-5-yl)pyrazolo[1,5-a]pyridin-2-yl)cyclopropanecarboxamide. To a 50 mL round bottom flask charged with triphenylphosphine (251 mg, 0.96 mmol), Intermediate 8 (193 mg, 0.96 mmol) and N-(5-(4-hydroxy-1-methyl-1H-pyrazol-5-yl)pyrazolo[1,5-a]pyridin-2-yl)cyclopropanecarboxamide (190 mg, 0.64 mmol) was added THE (3.2 mL). To the resulting solution was added DIAD (0.19 mL, 0.96 mmol) dropwise. After addition of the DIAD, the solution was heated to 60° C. for 4 hours. The crude reaction was quenched with H₂O (2 mL) and then diluted with EtOAc (20 mL). The layers were separated, the organic layer was collected and washed with H₂O (20 mL×2). The organic layers were collected, dried over MgSO₄, filtered, and concentrated down under vacuo to afford an oil. The oil was purified via silica column using 20-100% EtOAc:Hexanes followed by a second silica column using 50-75% EtOAc:Hexanes. To the crude, clear residue was added DCM (1.64 mL) and TFA (0.5 mL). The reaction was complete after 1 hour at room temperature. The crude product was evaporated in vacuo. The crude oil residue was dissolved with DCM/MeOH and concentrated down again to afford 150 mg of oil. The material was dissolved with MeOH and purified via HPLC using 10-60% ACN-H₂O with 20 mM of NH₄OH as modifier. The fractions containing desired material were collected, frozen, and lyophilized to afford a white powder (150 mg, 47%). MS (ESI): mass calcd. for C₂₀H₂₄N₆O₂, 380.2. m/z found, 381.2, [M+H]⁺. ¹H NMR (400 MHz, Methanol-d4) δ 8.44 (dt, J=7.2, 1.0 Hz, 1H), 7.64 (dd, J=2.0, 0.9 Hz, 1H), 7.43 (s, 1H), 6.96 (dd, J=7.2, 1.9 Hz, 1H), 6.90 (s, 1H), 4.02 (d, J=5.6 Hz, 2H), 3.88 (s, 3H), 3.80-3.70 (m, 1H), 3.48 (t, J=8.1 Hz, 1H), 3.20 (t, J=7.9 Hz, 1H), 2.58 (hept, J=7.1 Hz, 1H), 1.86 (td, J=7.7, 3.9 Hz, 1H), 1.14 (d, J=6.8 Hz, 3H), 0.99 (dt, J=4.6, 3.1 Hz, 2H), 0.94-0.81 (m, 2H).

Step B: N-(5-(4-(((2R,3R)-1,3-Dimethylazetidin-2-yl)methoxy)-1-methyl-1H-pyrazol-5-yl)pyrazolo[1,5-a]pyridin-2-yl)cyclopropanecarboxamide. To a solution of N-(5-(1-methyl-4-(((2R,3R)-3-methylazetidin-2-yl)methoxy)-1H-pyrazol-5-yl)pyrazolo[1,5-a]pyridin-2-yl)cyclopropanecarboxamide (120 mg, 0.243 mmol) and H(CHO)n (17.5 mg, 0.194 mmol) was added NaCNBH₃ (45.8 mg, 0.728 mmol) and MeOH (2.0 mL) at room temperature overnight. LCMS showed the reaction was complete. The crude product was diluted with MeOH and purified via prep. HPLC using 10-60% ACN-H₂O with 20 mM of aq. NH₄OH as modifier. The pure fraction was collected, and lyophilized down to afford a white powder (28.8 mg, 30%). MS (ESI): mass calcd. for C₂₁H₂₆N₆O₂, 394.2. m/z found, 395.2 [M+H]⁺. ¹H NMR (400 MHz, Methanol-d4) δ 8.44 (dt, J=7.2, 1.0 Hz, 1H), 7.64 (dd, J=1.9, 0.9 Hz, 1H), 7.43 (s, 1H), 6.95 (dd, J=7.2, 2.0 Hz, 1H), 6.89 (s, 1H), 4.05 (dd, J=10.0, 4.1 Hz, 1H), 3.98 (dd, J=10.0, 7.2 Hz, 1H), 3.88 (s, 3H), 3.54 (t, J=7.2 Hz, 1H), 3.02 (td, J=7.6, 4.1 Hz, 1H), 2.52 (dd, J=8.7, 7.1 Hz, 1H), 2.31 (s, 4H), 1.86 (tt, J=8.0, 4.5 Hz, 1H), 1.07 (d, J=6.7 Hz, 3H), 1.04-0.95 (m, 2H), 0.94-0.82 (m, 2H).

### Example 25: (1S,2S)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide

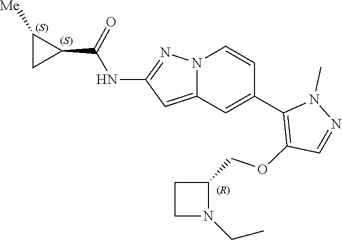

Step A: tert-butyl (R)-2-(((1-methyl-5-(2-((1S,2S)-2-methylcyclopropane-1-carboxamido)pyrazolo[1,5-a]pyridin-5-yl)-1H-pyrazol-4-yl)oxy)methyl)azetidine-1-carboxylate. To a mixture of tert-butyl (R)-2-(((5-(2-aminopyrazolo[1,5-a]pyridin-5-yl)-1-methyl-1H-pyrazol-4-yl)oxy)methyl)azetidine-1-carboxylate (150 mg, 0.376 mmol), (1S,2S)-2-methylcyclopropane-1-carboxylic acid (56.5 mg, 0.565 mmol) in DMF (10 mL), iPrNEt2 (0.389 mL, 2.25 mmol), and BOP (687 mg, 1.51 mmol) were added. The resultant mixture was stirred at room temperature for 16 hours. The mixture was worked-up with EtOAc/water/brine. The combined organic layers were dried (MgSO₄) and concentrated. The residue was purified over silica gel (12 g Gold ISCO column, 20-100% EtOAc/hexanes, 30 min gradient) providing the title compound (130 mg, 71%). MS (ESI): mass calcd. for C₂₅H₃₂N₆O₄, 480.2. m/z found, 481.2 [M+H]⁺.

Step B: (1S,2S)—N-(5-(4-(((R)-azetidin-2-yl)methoxy)-1-methyl-1H-pyrazol-5-yl)pyrazolo[1,5-a]pyridin-2-yl)-2-methylcyclopropane-1-carboxamide. Trifluoroacetic acid (5 mL) was added to a solution of tert-butyl (R)-2-(((1-methyl-5-(2-((1S,2S)-2-methylcyclopropane-1-carboxamido)pyrazolo[1,5-a]pyridin-5-yl)-1H-pyrazol-4-yl)oxy)methyl)azetidine-1-carboxylate (130 mg, 0.271 mmol)) in DCM (10 mL). The reaction mixture was stirred at room-temperature for 1 hour. The reaction mixture was concentrated to dryness under reduced pressure to give the crude product which was purified by reverse phase HPLC to afford pure product as an off-white solid (100 mg, 97%). MS (ESI): mass calcd. for C₂₂H₂₄N₆O₂, 380.196. m/z found, 381.2 [M+H]⁺.

Step C: (1S,2S)—N-(5-(4-(((R)-1-ethylazetidin-2-yl)methoxy)-1-methyl-1H-pyrazol-5-yl)pyrazolo[1,5-a]pyridin-2-yl)-2-methylcyclopropane-1-carboxamide. To (1S,2S)—N-(5-(4-(((R)-azetidin-2-yl)methoxy)-1-methyl-1H-pyrazol-5-yl)pyrazolo[1,5-a]pyridin-2-yl)-2-methylcyclopropane-1-carboxamide (100 mg, 0.263 mmol) in MeOH (10 mL) at room temperature were added acetaldehyde (12 mg, 0.289 mmol) and NaBH₃CN (36.3 mg, 0.578 mmol). The reaction mixture was stirred at room temperature for 10 minutes. The reaction mixture was treated with water (5 mL), stirred for 15 minutes, then extracted 2×50 mL 10% IPA/Chloroform. The combined organic layers were dried (MgSO₄) and concentrated. The residue was purified by reverse phase basic HPLC (H₂O, CH₃CN, NH₄OH). Fractions containing product were combined and concentrated to give the title compound as a white solid (81 mg, 75%). MS (ESI): mass calcd. for C₂₂H₂₈N₆O₂, 408.503. m/z found, 409.30 [M+H]⁺. ¹H NMR (400 MHz, Chloroform-d) δ 8.95 (s, 1H), 8.31 (dd, J=7.1, 1.1 Hz, 1H), 7.49 (dd, J=1.9, 0.9 Hz, 1H), 7.34 (s, 1H), 7.02 (s, 1H), 6.85 (dd, J=7.2, 1.9 Hz, 1H), 4.02 (dd, J=9.8, 6.4 Hz, 1H), 3.95 (dd, J=9.8, 4.8 Hz, 1H), 3.90 (s, 3H), 3.46-3.30 (m, 2H), 2.82-2.73 (m, 1H), 2.71-2.61 (m, 1H), 2.40-2.30 (m, 1H), 2.10-1.98 (m, 1H), 2.02-1.90 (m, 1H), 1.59-1.49 (m, 1H), 1.38-1.21 (m, 2H), 1.14 (d, J=6.0 Hz, 3H), 0.94 (t, J=7.2 Hz, 3H), 0.75-0.68 (m, 1H).

### Example 26: 1-methyl-N-[5-[2-methyl-4-[[(2R)-1-methylazetidin-2-yl]methoxy]pyrazol-3-yl]pyrazolo[1,5-a]pyridin-2-yl]pyrazole-4-carboxamide

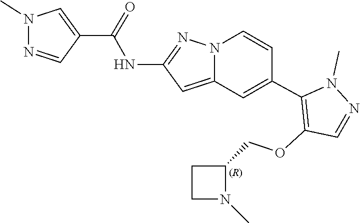

Step A: (R)-5-bromo-1-methyl-4-((1-methylazetidin-2-yl)methoxy)-1H-pyrazole. To the mixture of tert-butyl (R)-2-(((5-bromo-1-methyl-1H-pyrazol-4-yl)oxy)methyl)azetidine-1-carboxylate (4.85 g, 14.0 mmol) (Intermediate 6 step b) in MeOH (50 mL), HCl (4M in dioxane, 10 mL) was added. The mixture was stirred at RT for 16 h. then concentrated. The residue was re-dissolved into MeOH (50 mL), and H(CHO)n (1.89 g, 21 mmol) and NaBH₃CN (1.76 g, 28 mmol) were added to the reaction mixture. The resultant mixture was stirred at room temperature for 60 minutes. The reaction mixture was quenched with water (10 mL) and extracted with 10% MeOH/DCM (50 mL×2). The organic extracts were dried over anhydrous MgSO₄, filtered, and concentrated to dryness under reduced pressure to obtain the crude product, which was purified by flash column chromatography (acetone/CH₂Cl₂) to afford the title compound (1500 mg, 41%) as a white solid. MS (ESI): mass calcd. for C₉H₁₄BrN₃O, 259.03. m/z found, 260.0 [M+H]⁺.

Step B: (R)-5-(1-methyl-4-((1-methylazetidin-2-yl)methoxy)-1H-pyrazol-5-yl)pyrazolo[1,5-a]pyridin-2-amine. To a 40 mL vial was charged (R)-5-bromo-1-methyl-4-((1-methylazetidin-2-yl)methoxy)-1H-pyrazole (574 mg, 2.20 mmol), tert-butyl (5-(4,4,5,5-tetramethyl-1,3,2-dioxaborolan-2-yl)pyrazolo[1,5-a]pyridin-2-yl)carbamate (720 mg, 2.00 mmol), 1,1′-bis(diphenylphosphino)ferrocene-palladium(II)dichloride dichloromethane complex (130 mg, 0.200 mmol), 1,4-dioxane (14 mL)/H₂O (3 mL), and potassium fluoride (KF) (349 mg, 6.01 mmol mL). The vial was heated to 90° C. for 4 h. The crude reaction mixture was diluted with ethyl acetate then washed with water and brine. The organics were separated, dried with MgSO₄, filtered then concentrated to dryness. The residue was purified directly by column chromatography on silica gel using hexanes/ethyl acetate (50-100%) to provide the Boc protected intermediate. The Boc protected intermediate was re-dissolved into CH₂Cl₂ (10 mL), and TFA (1 mL) was added. The mixture was stirred at RT for 4 h. After concentration, the title compound was obtained (330 mg, 53%). MS (ESI): mass calcd. for C₁₆H₂₀N₆O, 312.17. m/z found, 313.1 [M+H]⁺.

Step C: 1-methyl-N-[5-[2-methyl-4-[[(2R)-1-methylazetidin-2-yl]methoxy]pyrazol-3-yl]pyrazolo[1,5-a]pyridin-2-yl]pyrazole-4-carboxamide. The mixture of (R)-5-(1-methyl-4-((1-methylazetidin-2-yl)methoxy)-1H-pyrazol-5-yl)pyrazolo[1,5-a]pyridin-2-amine (80.0 mg, 0.256 mmol), 1-methyl-1H-pyrazole-4-carboxylic acid (32.3 mg 0.256 mmol), HATU (146 mmol, 0.384 mmol) and Et₃N (0.107 mL, 0.768 mmol) in CH₂Cl₂ (10 mL) was stirred at RT for 16 h. The mixture was washed with NaHCO₃ saturated solution (5 mL) and washed by NaOH (1M, 5 mL). The organic layer was concentrated to dryness under reduced pressure to obtain the crude product, which was purified by flash column chromatography (MeOH/CH₂Cl₂) to afford the title compound (6 mg, 6%). MS (ESI): mass calcd. for C₂₁H₂₄N₈O₂, 420.475. m/z found, 421.1 [M+H]⁺. ¹H NMR (500 MHz, Methanol-d4) δ 8.45-8.28 (m, 1H), 8.15 (s, 1H), 7.97 (s, 1H), 7.59 (s, 1H), 7.34 (s, 1H), 6.97-6.82 (m, 2H), 3.98-3.86 (m, 5H), 3.80 (s, 3H), 3.41-3.33 (m, 1H), 3.29-3.26 (m, 1H), 2.86-2.77 (m, 1H), 2.22 (s, 3H), 2.01-1.84 (m, 2H).

### Example 27: N-[5-[4-[(2S)-2-hydroxy-2-phenyl-ethoxy]-2-methyl-pyrazol-3-yl]pyrazolo[1,5-a]pyridin-2-yl]cyclopropanecarboxamide

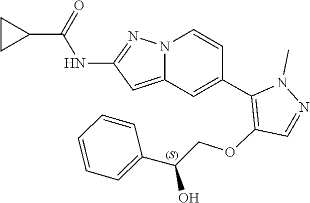

Intermediate 4 (200 mg, 0.673 mmol), (S)-2-phenyloxirane (88.9 mg, 0.740 mmol), Cs₂CO₃ (658 mg, 2.018 mmol), and DMF (3 mL) were added to a 5 mL microwave tube. The resultant mixture was stirred while heating at 80° C. via microwave irradiation for 1 h. The reaction mixture was purified by preparative HPLC using a Xtimate C18 250×50 mm×10 μm column (eluent: 30% to 60% (v/v) CH₃CN and H₂O with 0.05% NH₃) to afford pure product. The product was suspended in water (10 mL), the mixture frozen using dry ice/acetone, and then lyophilized to dryness to afford the title compound (56.1 mg, 19%) as a white solid. MS (ESI): mass calcd. for C₂₃H₂₃N₅O₃, 417.5. m/z found, 418.3 [M+H]⁺. ¹H NMR (400 MHz, DMSO-d6) δ 11.09 (s, 1H), 8.54 (d, J=7.2 Hz, 1H), 7.62 (d, J=0.4 Hz, 1H), 7.44 (s, 1H), 7.42-7.37 (m, 2H), 7.36-7.29 (m, 2H), 7.27 (d, J=7.2 Hz, 1H), 6.91 (dd, J=2.0, 7.2 Hz, 1H), 6.88 (s, 1H), 5.62 (br s, 1H), 4.86 (t, J=5.2 Hz, 1H), 4.08-3.94 (m, 2H), 3.85 (s, 3H), 2.01-1.87 (m, 1H), 0.90-0.77 (m, 4H)

### Example 28: N-[5-[5-methyl-3-[[(2S)-morpholin-2-yl]methoxy]isoxazol-4-yl]pyrazolo[1,5-a]pyridin-2-yl]cyclopropanecarboxamide

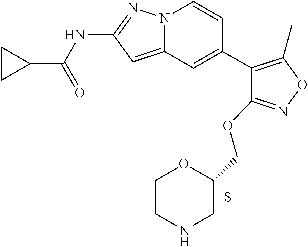

Step A: N-(5-(3-hydroxy-5-methylisoxazol-4-yl)pyrazolo[1,5-a]pyridin-2-yl)cyclopropanecarboxamide. To a vial containing N-(5-(4,4,5,5-tetramethyl-1,3,2-dioxaborolan-2-yl)pyrazolo[1,5-a]pyridin-2-yl)cyclopropanecarboxamide (920 mg, 2.81 mmol), 4-bromo-5-methylisoxazol-3-ol (500 mg, 2.81 mmol), Ruphos-G2 (110 mg, 0.14 mmol), K₃PO₄ (0.5 M in water, 9.0 mL, 0.85 mmol) and 1,4-dioxane (6.70 mL, 0.3 M) was evacuated and backfilled with nitrogen (3×). The vial was then placed in a pre-heated heating block at 90° C. After 1 hour, the solution was cooled to 23° C. and was added EtOAc (4 mL) followed by water (8 mL). The layers were separated and the aqueous was extracted with EtOAc (2×4 mL). The aqueous layer was acidified with 1N HCl until pH=5. The slurry was filtered and washed with water (2×2 mL) to afford the title compound (85 mg, 0.285 mmol, 10% yield) as a beige solid.

Step B: Tert-butyl (S)-2-(((4-(2-(cyclopropanecarboxamido)pyrazolo[1,5-a]pyridin-5-yl)-5-methylisoxazol-3-yl)oxy)methyl)morpholine-4-carboxylate. A microwave vial containing N-(5-(3-hydroxy-5-methylisoxazol-4-yl)pyrazolo[1,5-a]pyridin-2-yl)cyclopropanecarboxamide (40 mg, 0.134 mmol), tert-butyl (S)-2-((tosyloxy)methyl)morpholine-4-carboxylate (55 mg, 0.150 mmol), cesium carbonate (97.0 mg, 0.295 mmol) in DMF (1.25 mL) was heated at 80° C. for 1 hour. The resulting mixture was cooled to 23° C. and then was added water (2 mL) followed by EtOAc (2 mL). The layers were separated and the aqueous was extracted with EtOAc (3×4 mL) and the combined organics were dried with MgSO₄, filtered and concentrated in vacuo. The resulting oil was purified by silica gel chromatography (0-80% EtOAc in hexanes) to give the title compound (22 mg, 0.044 mmol, 32% yield) as a clear colorless oil which was used as is in the following reaction.

Step C: N-[5-[5-methyl-3-[[(2S)-morpholin-2-yl]methoxy]isoxazol-4-yl]pyrazolo[1,5-a]pyridin-2-yl]cyclopropanecarboxamide. To a vial containing tert-butyl (S)-2-(((4-(2-(cyclopropanecarboxamido)pyrazolo[1,5-a]pyridin-5-yl)-5-methylisoxazol-3-yl)oxy)methyl)morpholine-4-carboxylate (22 mg, 0.044 mmol) was added trifluoroacetic acid (0.41 mL) and the resulting mixture allowed to stand at 23° C. for 10 minutes. The solvent was removed in vacuo at the resulting oil was purified by reverse phase using 10-80% MeCN in water containing 20 mM NH₄OH (C18 50×21 mm). The fractions containing the desired product were pooled together and lyophilized to provide the title compound (17 mg, 0.043 mmol, 97% yield) MS (ESI): mass calcd. for C₂₀H₂₃N₅O₄, 397.4. m/z found, 398.2 [M+H]⁺. ¹H NMR (500 MHz, DMSO-d6) δ 11.05 (s, 1H), 8.58 (d, J=7.2, 1H), 7.64 (dd, J=2.0, 0.9 Hz, 1H), 6.90 (dd, J=7.2, 2.0 Hz, 1H), 6.85 (s, 1H), 4.27-4.17 (m, 2H), 3.84-3.64 (m, 2H), 3.51-3.40 (m, 1H), 2.87 (dd, J=12.1, 2.5 Hz, 1H), 2.72-2.60 (m, 2H), 2.54 (s, 1H), 2.52 (s, 3H), 1.98-1.86 (m, 1H), 0.92-0.75 (m, 4H).

### Example 29: N-[5-[3-[(3-methoxy-1-methyl-azetidin-3-yl)methoxy]-5-methyl-isoxazol-4-yl]pyrazolo[1,5-a]pyridin-2-yl]cyclopropanecarboxamide

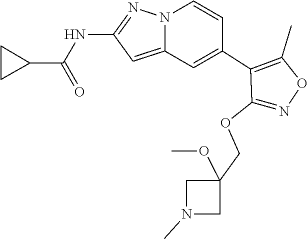 [SMILES: COC1(COc2noc(C)c2-c2ccn3nc(NC(=O)C4CC4)cc3c2)CN(C)C1]

H(CHO)n (22 mg, 0.245 mmol) was added to a solution containing N-[5-[3-[(3-methoxyazetidin-3-yl)methoxy]-5-methyl-isoxazol-4-yl]pyrazolo[1,5-a]pyridin-2-yl]cyclopropanecarboxamide and MeOH (1.6 mL). The reaction mixture was stirred at room temperature then NaBH₃CN (31 mg, 0.49 mmol) was added to the reaction mixture. The resultant mixture was stirred at room temperature for 60 minutes. The reactant mixture was concentrated in vacuo and the crude material was purified by reverse phase HPLC basic (C18 50×21 mm) using 10-100% MeCN in water (20 mM NH₄OH) and lyophilized to afford the title compound (49 mg, 73%) as a white solid. MS (ESI): mass calcd. for C₂₁H₂₅N₅O₄, 411.5. m/z found, 412.3 [M+H]⁺. ¹H NMR (500 MHz, DMSO-d6) δ 11.05 (s, 1H), 8.57 (d, J=7.2 Hz, 1H), 7.64 (d, J=1.8 Hz, 1H), 6.90 (dd, J=7.2, 1.9 Hz, 1H), 6.83 (s, 1H), 4.52 (s, 2H), 3.37-3.33 (m, 2H), 3.19 (s, 3H), 2.97-2.88 (m, 2H), 2.53 (s, 3H), 2.27 (s, 3H), 1.96-1.88 (m, 1H), 0.88-0.77 (m, 4H).

### Example 30: N-(2,6-dimethylpyrimidin-4-yl)-5-[3-[(3-methoxyazetidin-3-yl)methoxy]-5-methyl-isoxazol-4-yl]pyrazolo[1,5-a]pyridin-2-amine

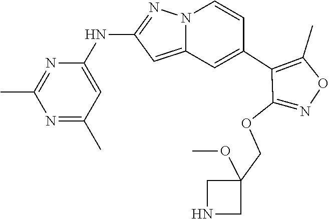 [SMILES: COC1(COc2noc(C)c2-c2ccn3nc(Nc4cc(C)nc(C)n4)cc3c2)CNC1]

Step A and B: Tert-butyl 3-(((4-(2-((tert-butoxycarbonyl)amino)pyrazolo[1,5-a]pyridin-5-yl)-5-methylisoxazol-3-yl)oxy)methyl)-3-methoxyazetidine-1-carboxylate. The title compound (5.5 mg, 4%) was prepared as described in Example 31 except tert-butyl 3-methoxy-3-((tosyloxy)methyl)azetidine-1-carboxylate was used instead of (S)-2-((tert-butoxycarbonyl)amino)-2-phenylethyl 4-methylbenzenesulfonate in step A; tert-butyl (5-(4,4,5,5-tetramethyl-1,3,2-dioxaborolan-2-yl)pyrazolo[1,5-a]pyridin-2-yl)carbamate was used instead of N-(5-(4,4,5,5-tetramethyl-1,3,2-dioxaborolan-2-yl)pyrazolo[1,5-a]pyridin-2-yl)cyclopropanecarboxamide in step B to give tert-butyl 3-(((4-(2-((tert-butoxycarbonyl)amino)pyrazolo[1,5-a]pyridin-5-yl)-5-methylisoxazol-3-yl)oxy)methyl)-3-methoxyazetidine-1-carboxylate which was used as in the following reaction.

Step C: 5-(3-((3-methoxyazetidin-3-yl)methoxy)-5-methylisoxazol-4-yl)pyrazolo[1,5-a]pyridin-2-amine. To a vial containing tert-butyl 3-(((4-(2-((tert-butoxycarbonyl)amino)pyrazolo[1,5-a]pyridin-5-yl)-5-methylisoxazol-3-yl)oxy)methyl)-3-methoxyazetidine-1-carboxylate (520 mg, 0.98 mmol) was added trifluoroacetic acid (2.6 mL) and the resulting mixture allowed to stand at 23° C. for 10 minutes. The solvent was removed in vacuo at the resulting oil was purified by reverse phase using 10-80% MeCN in water containing 20 mM NH₄OH. The fractions containing the desired product were pooled together and lyophilized to provide the title compound (170 mg, 0.47 mmol, 47% yield). MS (ESI): mass calcd. for C₁₆H₁₉N₅O₃, 329.4. m/z found, 330.2 [M+H]⁺.

Step D: N-(2,6-dimethylpyrimidin-4-yl)-5-[3-[(3-methoxyazetidin-3-yl)methoxy]-5-methyl-isoxazol-4-yl]pyrazolo[1,5-a]pyridin-2-amine. To a vial charged with 5-(3-((3-methoxyazetidin-3-yl)methoxy)-5-methylisoxazol-4-yl)pyrazolo[1,5-a]pyridin-2-amine (40 mg, 0.121 mmol), 4-chloro-2,6-dimethylpyrimidine (43 mg, 0.304 mmol), Brettphos-Pd-G3 (4.5 mg, 0.005 mmol), cesium carbonate (152 mg, 0.47 mmol) and 1,4-dioxane (0.5 mL) was evacuated and backfilled with Argon. This was repeated two more times and the resulting solution was then stirred at 65 C. After 2 hours, the reaction mixture was cooled to 23 C and then filtered through celite and the solvent removed in vacuo. The resulting oil was purified by reverse phase using 10-80% MeCN in water containing 20 mM NH₄OH. The fractions containing the desired product were pooled together and lyophilized to provide the title compound (5.5 mg, 0.013 mmol, 10% yield). MS (ESI): mass calcd. for C₂₂H₂₅N₇O₃, 435.5. m/z found, 436.3 [M+H]⁺. ¹H NMR (500 MHz, DMSO-d6) δ 10.17 (s, 1H), 8.59 (d, J=7.2 Hz, 1H), 7.63 (t, J=1.3 Hz, 1H), 6.99 (s, 1H), 6.86 (dd, J=7.2, 2.0 Hz, 1H), 6.82 (s, 1H), 4.57 (s, 2H), 3.53 (d, J=8.4 Hz, 2H), 3.20 (s, 2H), 2.54 (s, 3H), 2.45 (s, 3H), 2.29 (s, 3H), 2.07 (s, 3H).

### Example 31: N-[5-[3-[(2S)-2-amino-2-phenyl-ethoxy]-5-methyl-isoxazol-4-yl]pyrazolo[1,5-a]pyridin-2-yl]cyclopropanecarboxamide

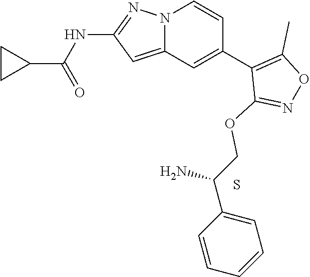

Step A: Tert-butyl (S)-(2-((4-bromo-5-methylisoxazol-3-yl)oxy)-1-phenylethyl)carbamate. A microwave vial containing 4-bromo-5-methylisoxazol-3-ol (500 mg, 2.80 mmol), (S)-2-((tert-butoxycarbonyl)amino)-2-phenylethyl 4-methylbenzenesulfonate (1154 mg, 2.95 mmol), cesium carbonate (2000 mg, 6.18 mmol) in DMF (3.9 mL, 0.70 M) was heated at 80° C. for 18 hours. The resulting mixture was cooled to 23° C. and then was added water (4 mL) followed by EtOAc (8 mL). The layers were separated and the aqueous was extracted with EtOAc (3×8 mL) and the combined organics were dried with MgSO₄, filtered and concentrated in vacuo. The resulting oil was purified by silica gel chromatography (0-80% EtOAc in hexanes) to give the title compound (240 mg, 0.60 mmol, 21% yield) as a clear colorless oil which was used as is in the following reaction.

Step B: Tert-butyl (S)-(2-((4-(2-(cyclopropanecarboxamido)pyrazolo[1,5-a]pyridin-5-yl)-5-methylisoxazol-3-yl)oxy)-1-phenylethyl)carbamate. To a vial containing N-(5-(4,4,5,5-tetramethyl-1,3,2-dioxaborolan-2-yl)pyrazolo[1,5-a]pyridin-2-yl)cyclopropanecarboxamide (240 mg, 0.725 mmol), tert-butyl (S)-(2-((4-bromo-5-methylisoxazol-3-yl)oxy)-1-phenylethyl)carbamate (240 mg, 0.60 mmol), PdCl₂(dppf) (12.0 mg, 0.016 mmol, 2.7 mol %), K₃PO₄ (0.5 M in water, 1.70 mL, 0.85 mmol) and 1,4-dioxane (2.00 mL, 0.3 M) was evacuated and backfilled with nitrogen (3×). The vial was then placed in a pre-heated heating block at 60° C. After 18 hour, the solution was cooled to 23 C and was added EtOAc (4 mL) followed by water (2 mL). The layers were separated and the aqueous was extracted with EtOAc (2×4 mL) and the combined organics were dried with MgSO4, filtered and concentrated in vacuo. The residue was purified by flash column chromatography (10-100% EtOAc in hexanes) and the fractions containing product were combined and concentrated to afford title compound (194 mg, 0.375 mmol, 62% yield) as an oil which was used as is in the following reaction.

Step C: To a vial containing tert-butyl (S)-(2-((4-(2-(cyclopropanecarboxamido)pyrazolo[1,5-a]pyridin-5-yl)-5-methylisoxazol-3-yl)oxy)-1-phenylethyl)carbamate (194 mg, 0.375 mmol) was added trifluoroacetic acid (1.0 mL) and the resulting mixture allowed to stand at 23° C. for 10 minutes. The solvent was removed in vacuo at the resulting oil was purified by reverse phase using 10-80% MeCN in water containing 20 mM NH₄OH. The fractions containing the desired product were pooled together and lyophilized to provide the title compound (64.3 mg, 0.15 mmol, 40% yield). MS (ESI): mass calcd. for C₂₃H₂₃N₅O₃, 417.5. m/z found, 418.2 [M+H]⁺. ¹H NMR (500 MHz, DMSO-d6) δ 11.04 (s, 1H), 8.50 (d, J=7.3 Hz, 1H), 7.52-7.42 (m, 3H), 7.38-7.31 (m, 2H), 7.27 (dd, J=7.1, 1.7 Hz, 1H), 6.83 (s, 1H), 6.78 (dd, J=7.3, 2.2 Hz, 1H), 4.36-4.25 (m, 4H), 2.51 (s, 3H), 1.98-1.88 (m, 1H), 0.88-0.78 (m, 4H).

### Example 32: 5-[2-(difluoromethyl)-4-[(3R,4S)-4-fluoropyrrolidin-3-yl]oxy-pyrazol-3-yl]-N-(2,6-dimethylpyrimidin-4-yl)pyrazolo[1,5-a]pyridin-2-amine

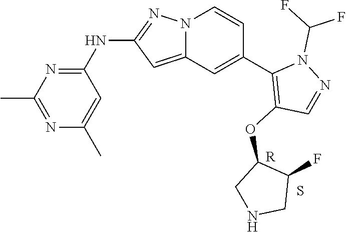

Step A: Tert-butyl (3R,4S)-3-((1-(difluoromethyl)-1H-pyrazol-4-yl)oxy)-4-fluoropyrrolidine-1-carboxylate. To a microwave vial charged with 1-(difluoromethyl)-1H-pyrazol-4-ol (1.25 g, 9.32 mmol), tert-butyl (3S,4S)-3-fluoro-4-(tosyloxy)pyrrolidine-1-carboxylate (3.35 g, 9.32 mmol), Cs₂CO₃ (6.68 g, 20.5 mmol) in DMF (10 mL) was heated at 150° C. for 1 hour. The reaction mixture was then cooled to 23° C. and diluted with water (40 mL). The aqueous was extracted with EtOAc (3×40 mL) and the combined organics were washed with sat. aq. NaCl (2×30 mL), then dried with MgSO4, filtered and concentrated in vacuo. The resulting crude oil was purified by silica gel chromatography (0-100%) EtOAc in hexanes to give tert-butyl (3R,4S)-3-((1-(difluoromethyl)-1H-pyrazol-4-yl)oxy)-4-fluoropyrrolidine-1-carboxylate (390 mg, 13%).

Step B: Tert-butyl (3R,4S)-3-((5-bromo-1-(difluoromethyl)-1H-pyrazol-4-yl)oxy)-4-fluoropyrrolidine-1-carboxylate. tert-butyl (3R,4S)-3-((1-(difluoromethyl)-1H-pyrazol-4-yl)oxy)-4-fluoropyrrolidine-1-carboxylate (390 mg, 1.21 mmol) in acetonitrile (5 mL) was added N-bromosuccinimide (238 mg, 1.34 mmol) and the resulting mixture stirred at 23° C. for 18 hours. Additional N-bromosuccinimide (30 mg, 0.17 mmol) was added and stirred for an additional 5 hours at 23° C. The mixture was then concentrated in vacuo and purified by silica gel chromatography (0-100%) EtOAc in hexanes to give tert-butyl (3R,4S)-3-((5-bromo-1-(difluoromethyl)-1H-pyrazol-4-yl)oxy)-4-fluoropyrrolidine-1-carboxylate (360 mg, 74%).

Step C: Tert-butyl (3R,4S)-3-((1-(difluoromethyl)-5-(2-((2,6-dimethylpyrimidin-4-yl)amino)pyrazolo[1,5-a]pyridin-5-yl)-1H-pyrazol-4-yl)oxy)-4-fluoropyrrolidine-1-carboxylate. To a vial was charged tert-butyl (3R,4S)-3-((5-bromo-1-(difluoromethyl)-1H-pyrazol-4-yl)oxy)-4-fluoropyrrolidine-1-carboxylate (100 mg, 0.25 mmol), Intermediate 11 (100 mg, 0.27 mmol), 1,1′-bis(diphenylphosphino)ferrocene-palladium(ii)dichloride dichloromethane complex (4.5 mg, 0.0063 mmol), 1,4-dioxane (0.8 mL), and 0.5 M aq. K₃PO₄ (0.7 mL). The vial was sealed then evacuated and backfilled with N₂ several times. The vial was heated to 60° C. for 1 h. The reaction as cooled to 23° C. and diluted with water (2 mL) and extracted with 10% MeOH in DCM (4×3 mL). The combined organics were dried with MgSO₄, filtered then concentrated to dryness. The residue was purified by silica column chromatography using MeOH in DCM (0-10%) to provide tert-butyl (3R,4S)-3-((1-(difluoromethyl)-5-(2-((2,6-dimethylpyrimidin-4-yl)amino)pyrazolo[1,5-a]pyridin-5-yl)-1H-pyrazol-4-yl)oxy)-4-fluoropyrrolidine-1-carboxylate (77 mg, 55%).

Step D: 5-[2-(difluoromethyl)-4-[(3R,4S)-4-fluoropyrrolidin-3-yl]oxy-pyrazol-3-yl]-N-(2,6-dimethylpyrimidin-4-yl)pyrazolo[1,5-a]pyridin-2-amine. To a solution of tert-butyl (3R,4S)-3-((1-(difluoromethyl)-5-(2-((2,6-dimethylpyrimidin-4-yl)amino)pyrazolo[1,5-a]pyridin-5-yl)-1H-pyrazol-4-yl)oxy)-4-fluoropyrrolidine-1-carboxylate (80 mg, 0.14 mmol) in DCM (0.5 mL) was added trifluoroacetic acid (0.11 mL, 1.43 mmol). The mixture was stirred at 23° C. for 2 hours and then concentrated in vacuo. The residue was purified via prep HPLC (Basic ISCO, 10-80% AcN/20 mM aq NH₄OH) and lyophilized to yield the title compound as a white solid (64 mg, 90%). MS (ESI): mass calcd. for C₂₁H₂₁F₃N₈O, 458.2. m/z found, 459.2 [M+H]⁺. ¹H NMR (500 MHz, DMSO-d6) δ 10.23 (s, 1H), 8.66 (d, J=7.1 Hz, 1H), 8.03 (s, 1H), 7.87-7.59 (m, 2H), 6.98 (s, 1H), 6.94 (s, 1H), 6.83 (dd, J=7.1, 2.0 Hz, 1H), 5.27-5.07 (m, 1H), 4.70-4.55 (m, 1H), 3.23-3.07 (m, 2H), 2.99-2.87 (m, 1H), 2.76 (dd, J=11.2, 7.6 Hz, 1H), 2.46 (s, 3H), 2.30 (s, 3H).

### Example 33: 6-[[5-[2-(difluoromethyl)-4-[[(2R)-1-methylazetidin-2-yl]methoxy]pyrazol-3-yl]pyrazolo[1,5-a]pyridin-2-yl]amino]-2,4-dimethyl-pyridazin-3-one

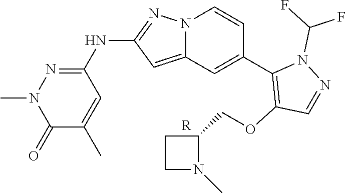

Step A: Tert-butyl (R)-2-(((1-(difluoromethyl)-5-(2-((1,5-dimethyl-6-oxo-1,6-dihydropyridazin-3-yl)amino)pyrazolo[1,5-a]pyridin-5-yl)-1H-pyrazol-4-yl)oxy)methyl)azetidine-1-carboxylate. A mixture of consisting of Intermediate 12 (300 mg, 0.70 mmol), 6-chloro-2,4-dimethylpyridazin-3(2H)-one (131 mg, 0.83 mmol), tBuBrettphos-Pd-G3 (59 mg, 0.07 mmol), Cs₂CO₃ (675 mg, 2.07 mmol) and 1,4-dioxane (1.4 mL) was sonicated under vacuum for 2 minutes, then vented with Argon. The mixture was stirred at 90° C. for 7 hours before being cooled to 23° C. The mixture was filtered through celite and concentrated to dryness in vacuo to give the crude product, which was purified by silica gel chromatography (0-10%) MeOH in DCM to give tert-butyl (R)-2-(((1-(difluoromethyl)-5-(2-((1,5-dimethyl-6-oxo-1,6-dihydropyridazin-3-yl)amino)pyrazolo[1,5-a]pyridin-5-yl)-1H-pyrazol-4-yl)oxy)methyl)azetidine-1-carboxylate (340 mg, 88% yield).

Step B: 6-[[5-[2-(difluoromethyl)-4-[[(2R)-1-methylazetidin-2-yl]methoxy]pyrazol-3-yl]pyrazolo[1,5-a]pyridin-2-yl]amino]-2,4-dimethyl-pyridazin-3-one. A solution of tert-butyl (R)-2-(((1-(difluoromethyl)-5-(2-((1,5-dimethyl-6-oxo-1,6-dihydropyridazin-3-yl)amino)pyrazolo[1,5-a]pyridin-5-yl)-1H-pyrazol-4-yl)oxy)methyl)azetidine-1-carboxylate (170 mg, 0.31 mmol) in DCM (0.4 mL) was added trifluoroacetic acid (0.45 mL, 6.10 mmol) and stirred at 23° C. After 30 minutes, the reaction mixture was concentrated in vacuo. The crude oil was dissolved in 10% MeOH in DCM (5 mL) and then 2N potassium carbonate (3 mL) and water (3 mL) was added. The layers were separated and the aqueous was extracted with 10% MeOH in DCM (3×6 mL). The combined organics were dried with MgSO4, filtered and concentrated in vacuo. The crude material was then dissolved in MeOH (1 mL) and 37% aq. formaldehyde (37 mg, 0.45 mmol) was added followed by sodium cyanoborohydride (57 mg, 0.92 mmol). The mixture was stirred at 23° C. for 2 hours and then concentrated in vacuo. The residue was purified via prep HPLC (Basic ISCO, 10-80% AcN/20 mM aq NH₄OH) and lyophilized to yield the title compound as a white solid (84 mg, 58%). MS (ESI): mass calcd. for C₂₂H₂₄F₂N₈O₂, 470.2. m/z found, 471.3 [M+H]⁺. ¹H NMR (600 MHz, DMSO-d6) δ 9.77 (s, 1H), 8.60 (dt, J=7.2, 1.0 Hz, 1H), 7.95 (s, 1H), 7.85-7.59 (m, 2H), 7.22 (q, J=1.2 Hz, 1H), 6.90 (d, J=0.9 Hz, 1H), 6.82 (dd, J=7.1, 2.0 Hz, 1H), 4.05 (dd, J=10.2, 4.2 Hz, 1H), 4.00 (dd, J=10.3, 6.5 Hz, 1H), 3.62 (s, 3H), 3.22 (d, J=7.7 Hz, 2H), 2.71 (d, J=8.6 Hz, 1H), 2.17 (s, 3H), 2.09 (d, J=1.3 Hz, 3H), 1.95-1.88 (m, 1H), 1.84 (p, J=9.0 Hz, 1H).

### Example 34: (*S)-3-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one

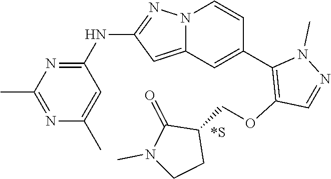 [SMILES: Cc1cc(Nc2cc3cc(-c4c(OC[C@@H]5CCN(C)C5=O)cnn4C)ccn3n2)nc(C)n1]

To a 40 mL vial was charged Intermediate 10 (200 mg, 0.596 mmol) and triphenylphosphine (323 mg, 0.1.23 mmol) as solids. The vial was evacuated and backfilled with N₂ 3×. Then, a solution of 3-(hydroxymethyl)-1-methylpyrrolidin-2-one (123 mg, 0.952 mmol) in THE (3 mL) was then added to the vial in one-portion. The vial was cooled using an ice/water bath for 5 min and then diisopropyl azodicarboxylate (0.24 mL, 1.19 mmol) was added dropwise. The ice-bath was removed, and the reaction allowed to stir at room temperature overnight. The reaction mixture was then poured into 40 mL sat. aq. NaHCO₃. The aq. Mixture was extracted with EA (3×40 mL), dried with anh. MgSO₄, filtered, and conc to dryness. The crude was purified via prep HPLC: Xbridge C18 50×100 5 uM 0-100% ACN/20 mM NH₄OH (20 min). The pure fractions were collected and lyophilized down to afford the racemate (48 mg, 18%). The racemate was separated using SFC with AD-H (2×25 cm) column, 40% methanol (0.1% EA)/CO2 gradient, 65 mL/min to afford the pure isomer (20 mg, 7.5%) as the second eluting peak. MS (ESI): mass calcd. for C₂₃H₂₆N₈O₂, 446.2. m/z found, 447.2 [M+H]⁺. ¹H NMR (500 MHz, DMSO-d6) δ 10.19 (s, 1H), 8.64-8.57 (m, 1H), 7.66 (dd, J=2.0, 0.9 Hz, 1H), 7.46 (s, 1H), 7.00 (s, 1H), 6.92-6.82 (m, 2H), 4.15-4.02 (m, 2H), 3.87 (s, 3H), 3.29-3.21 (m, 2H), 2.78-2.66 (m, 4H), 2.46 (s, 3H), 2.30 (s, 3H), 2.18-2.08 (m, 1H), 1.97-1.86 (m, 1H).

### Example 35: *R-4-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one

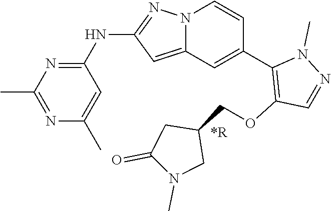 [SMILES: Cc1cc(Nc2cc3cc(-c4c(OC[C@@H]5CC(=O)N(C)C5)cnn4C)ccn3n2)nc(C)n1]

The title compound was prepared as described in Example 35 and isolated as the first eluting peak (17 mg, 6.4%). MS (ESI): mass calcd. for C₂₃H₂₆N₈O₂, 446.2. m/z found, 447.3 [M+H]⁺. ¹H NMR (500 MHz, DMSO-d6) δ 10.20 (s, 1H), 8.60 (dd, J=7.1, 1.0 Hz, 1H), 7.67 (dd, J=2.0, 0.9 Hz, 1H), 7.47 (s, 1H), 7.00 (s, 1H), 6.94-6.85 (m, 2H), 3.93 (d, J=6.5 Hz, 2H), 3.86 (s, 3H), 3.48-3.41 (m, 1H), 3.15 (dd, J=9.8, 5.2 Hz, 1H), 2.75-2.64 (m, 4H), 2.46 (s, 3H), 2.37 (dd, J=16.8, 9.4 Hz, 1H), 2.30 (s, 3H), 2.06 (dd, J=16.7, 6.1 Hz, 1H).

### Example 36: N-(2-cyano-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide

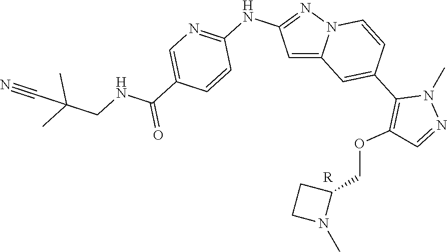

Step A. Methyl (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinate. A mixture of Intermediate 6 (400 mg, 1.00 mmol), methyl 6-bromonicotinate (282 mg, 1.31 mmol), Xantphos (116 mg, 0.20 mmol), palladium (II) acetate (23 mg, 0.10 mmol), cesium fluoride (457 mg, 3.01 mmol), and 1,4-dioxane (5 mL) was heated overnight at 100° C. under N₂ atmosphere. The reaction mixture was concentrated under reduced pressure to give the product, which was purified by FCC (eluent: petroleum ether:ethyl acetate=1:0 to 10:8) to give methyl (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinate as a yellow solid (446 mg, 65%).

Step B. (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinic acid. A solution consisting of (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinate (446 mg, 0.84 mmol), lithium hydroxide monohydrate (100 mg, 4.18 mmol), methanol (3 mL), THE (3 mL), and water (0.5 mL) was stirred for 1 hr at room temperature. The reaction mixture was concentrated under reduced pressure to afford the crude product, (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinic acid, as a yellow solid (400 mg, 75%).

Step C. Tert-butyl (R)-2-(((5-(2-((5-((2-cyano-2-methylpropyl)carbamoyl)pyridin-2-yl)amino)pyrazolo[1,5-a]pyridin-5-yl)-1-methyl-1H-pyrazol-4-yl)oxy)methyl)azetidine-1-carboxylate. A mixture consisting of (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)nicotinic acid (200 mg, 0.39 mmol), 3-amino-2,2-dimethylpropanenitrile (38 mg, 0.39 mmol), HATU (293 mg, 0.77 mmol), DIEA (149 mg, 1.16 mmol) and DMF (5 mL) was stirred for 1 hr at room temperature. The reaction was quenched with water (20 mL) and the suspension isolated via filtration. The filter cake was washed with water (10 mL×2) before drying under reduced pressure to afford the crude product, tert-butyl (R)-2-(((5-(2-((5-((2-cyano-2-methylpropyl)carbamoyl)pyridin-2-yl)amino)pyrazolo[1,5-a]pyridin-5-yl)-1-methyl-1H-pyrazol-4-yl)oxy)methyl)azetidine-1-carboxylate (200 mg, 87%).

Step D. (R)-6-((5-(4-(azetidin-2-ylmethoxy)-1-methyl-1H-pyrazol-5-yl)pyrazolo[1,5-a]pyridin-2-yl)amino)-N-(2-cyano-2-methylpropyl)nicotinamide. A solution consisting of tert-butyl (R)-2-(((5-(2-((5-((2-cyano-2-methylpropyl)carbamoyl)pyridin-2-yl)amino)pyrazolo[1,5-a]pyridin-5-yl)-1-methyl-1H-pyrazol-4-yl)oxy)methyl)azetidine-1-carboxylate (200 mg, 0.33 mmol), trifluoroacetic acid (2 mL, 26.92 mmol), and dichloromethane (6 mL) was stirred for 1 hr at room temperature. The reaction mixture was concentrated under reduced pressure to afford the crude product, (R)-6-((5-(4-(azetidin-2-ylmethoxy)-1-methyl-1H-pyrazol-5-yl)pyrazolo[1,5-a]pyridin-2-yl)amino)-N-(2-cyano-2-methylpropyl)nicotinamide, as a yellow solid (300 mg, 180%).

Step E. (R)—N-(2-cyano-2-methylpropyl)-6-((5-(1-methyl-4-((1-methylazetidin-2-yl)methoxy)-1H-pyrazol-5-yl)pyrazolo[1,5-a]pyridin-2-yl)amino)nicotinamide. A solution consisting of (R)-6-((5-(4-(azetidin-2-ylmethoxy)-1-methyl-1H-pyrazol-5-yl)pyrazolo[1,5-a]pyridin-2-yl)amino)-N-(2-cyano-2-methylpropyl)nicotinamide (270 mg, 0.54 mmol), sodium cyanoborohydride, (68 mg, 1.08 mmol), paraformaldehyde (49 mg, 0.54 mmol) and methanol (4 mL) was stirred for 1 hr at room temperature. The reaction mixture was purified by preparative HPLC using a Boston Prime C18 150×30 mm×5 μm column (eluent: 28% to 58% (v/v) CH₃CN and NH₃H₂O) to afford pure product. The product was suspended in water (10 mL), the mixture frozen using dry ice/ethanol, and then lyophilized to dryness to afford the title compound (20 mg, crude) as a white solid. The compound was further purified by SFC over DAICEL CHIRALCEL OJ-H 250 mm×30 mm, 5 μm (eluent: 45% to 45% (v/v) supercritical CO₂ in EtOH and H₂O with 0.1% NH₃). The pure fractions were collected, and the volatiles were removed under reduced pressure. The product was suspended in water (10 mL), the mixture frozen using dry ice/acetone, and then lyophilized to dryness to afford the title compound, (R)—N-(2-cyano-2-methylpropyl)-6-((5-(1-methyl-4-((1-methylazetidin-2-yl)methoxy)-1H-pyrazol-5-yl)pyrazolo[1,5-a]pyridin-2-yl)amino)nicotinamide, as a yellow solid (13.3 mg, 4%). MS (ESI): mass calcd. for C₂₇H₃₁N₉O₂, 513.3. m/z found, 514.2 [M+H]⁺. ¹H NMR (400 MHz, DMSO-d6) δ 10.28 (s, 1H), 8.78-8.71 (m, 2H), 8.62 (d, J=7.2 Hz, 1H), 8.10 (dd, J=2.0, 8.8 Hz, 1H), 7.70 (s, 1H), 7.47 (s, 1H), 7.38 (d, J=8.8 Hz, 1H), 6.99-6.85 (m, 2H), 4.00 (s, 1H), 3.87 (s, 3H), 3.81-3.76 (m, 1H), 3.49-3.46 (m, 1H), 3.44-3.41 (m, 3H), 2.85-2.72 (m, 1H), 2.31 (s, 3H), 2.08-1.95 (m, 2H), 1.34 (s, 6H) ¹H NMR (400 MHz, CD3OD): 8.79 (d, J=2.0 Hz, 1H), 8.42 (d, J=7.2 Hz, 1H), 8.08 (dd, J=2.0, 8.8 Hz, 1H), 7.97 (s, 1H), 7.54-7.45 (m, 2H), 7.36 (s, 1H), 6.86 (dd, J=2.0, 7.2 Hz, 1H), 6.68-6.58 (m, 2H), 4.26-4.12 (m, 1H), 4.10-4.00 (m, 1H), 3.91 (s, 3H), 3.72-3.54 (m, 4H), 3.17-2.95 (m, 1H), 2.45 (s, 3H), 2.23-2.05 (m, 2H), 1.43 (s, 6H)

### Example 37: N-isopropyl-2,6-dimethoxy-4-[[5-[2-methyl-4-[[(2R)-1-methylazetidin-2-yl]methoxy]pyrazol-3-yl]pyrazolo[1,5-a]pyridin-2-yl]amino]benzamide

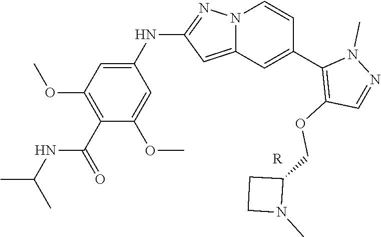

A mixture consisting of (R)-5-(1-methyl-4-((1-methylazetidin-2-yl)methoxy)-1H-pyrazol-5-yl)pyrazolo[1,5-a]pyridin-2-amine (110 mg, 0.352 mmol) (prepared as described in Step B of Example 26), 4-bromo-N-isopropyl-2,6-dimethoxybenzamide (138 mg, 0.458 mmol) (prepared as described in Example 120 Step B, except propan-2-amine was used instead of 3-amino-2,2-dimethylpropanenitrile), and Cs₂CO₃ (344 mg, 1.06 mmol) was dissolved in 1,4-dioxane (3 mL). The resultant mixture was sparged with N₂ for 2 minutes and then treated with Brettphos-Pd-G3 (57 mg, 0.063 mmol). The mixture was sparged with N₂ for another 2 minutes and then stirred for 3 hr at 120° C. before being cooled to room-temperature. The reaction mixture was concentrated to dryness under reduced pressure to give the crude product, which was further purified by preparative HPLC DG using a Phenomenex Gemini NX 150×30 mm×5 um column ((eluent: 24% to 54% (v/v) water (0.05% NH₃H₂O)-ACN to afford pure product, which was suspended in water (20 mL), the mixture frozen using dry ice/ethanol, and then lyophilized to dryness to afford the title compound, N-isopropyl-2,6-dimethoxy-4-[[5-[2-methyl-4-[[(2R)-1-methylazetidin-2-yl]methoxy]pyrazol-3-yl]pyrazolo[1,5-a]pyridin-2-yl]amino]benzamide, as a white solid (75.00 mg, 40%). MS (ESI): mass calcd. for C₂₈H₃₅N₇O₄, 533.3. m/z found, 534.1 [M+H]⁺. ¹H NMR (400 MHz, DMSO-d6) δ 9.17-9.11 (m, 1H), 8.58 (d, J=7.2 Hz, 1H), 7.68 (d, J=8.0 Hz, 1H), 7.58-7.55 (m, 1H), 7.45 (s, 1H), 6.90-6.81 (m, 3H), 6.18-6.13 (m, 1H), 4.00-3.88 (m, 3H), 3.86 (s, 3H), 3.77-3.69 (m, 6H), 3.27-3.19 (m, 2H), 2.74-2.66 (m, 1H), 2.19 (s, 3H), 1.98-1.87 (m, 1H), 1.86-1.78 (m, 1H), 1.13-0.97 (m, 6H)¹H NMR (400 MHz, DMSO-d6, T=80° C.): 8.78 (s, 1H), 8.50 (dd, J=0.8, 7.2 Hz, 1H), 7.56-7.50 (m, 1H), 7.38 (d, J=0.8 Hz, 1H), 7.25-7.18 (m, 1H), 6.84 (s, 2H), 6.82-6.79 (m, 1H), 6.16 (s, 1H), 4.04-3.94 (m, 1H), 3.91 (d, J=5.6 Hz, 2H), 3.83 (s, 3H), 3.74 (s, 6H), 3.27-3.18 (m, 2H), 2.74-2.67 (m, 1H), 2.19 (s, 3H), 1.98-1.90 (m, 1H), 1.88-1.78 (m, 1H), 1.11 (d, J=6.4 Hz, 6H)

### Example 38: N-[5-[2-methyl-4-[[(2R)-1-methylazetidin-2-yl]methoxy]pyrazol-3-yl]pyrazolo[1,5-a]pyridin-2-yl]cyclopropanecarboxamide

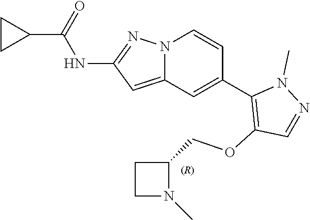

The title compound (350 mg, 75%) was prepared as described in Example 2 where (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) was used instead of (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine. MS (ESI): mass calcd. for C₂₀H₂₄N₆O₂, 380.2. m/z found, 381.3 [M+H]⁺. ¹H NMR (400 MHz, CDCl₃) δ 8.46 (s, 1H), 8.29 (d, J=7.2 Hz, 1H), 7.50 (d, J=0.8 Hz, 1H), 7.34 (s, 1H), 6.99 (s, 1H), 6.86 (dd, J=1.6, 7.2 Hz, 1H), 3.96 (d, J=5.2 Hz, 2H), 3.91 (s, 3H), 3.44-3.38 (m, 1H), 3.35-3.26 (m, 1H), 2.85-2.77 (m, 1H), 2.32 (s, 3H), 2.05-1.92 (m, 2H), 1.62-1.53 (m, 1H), 1.19-1.11 (m, 2H), 0.94-0.88 (m, 2H)

### Example 39: 5-[2-methyl-4-[[(2R)-1-methylazetidin-2-yl]methoxy]pyrazol-3-yl]-N-(6-methylpyridazin-3-yl)pyrazolo[1,5-a]pyridin-2-amine

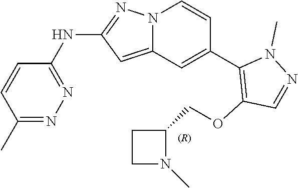

The title compound (46 mg, 3 steps, 20%) was prepared as described in Example 2 where 3-chloro-6-methylpyrimidine was used instead of 4-chloro-2,6-dimethylpyrimidine in Step A. MS (ESI): mass calcd. for C₂₁H₂₄N₈O, 404.2. m/z found, 405.2 [M+H]⁺. ¹H NMR (500 MHz, Chloroform-d) δ 9.72 (s, 1H), 8.33 (d, J=7.1 Hz, 1H), 7.97 (d, J=9.2 Hz, 1H), 7.40-7.35 (m, 1H), 7.27 (s, 1H), 7.20 (d, J=9.2 Hz, 1H), 6.72 (dd, J=7.1, 1.9 Hz, 1H), 6.66 (s, 1H), 3.89 (d, J=5.5 Hz, 2H), 3.83 (s, 3H), 3.33 (td, J=7.3, 2.8 Hz, 1H), 3.24 (td, J=8.0, 4.0 Hz, 1H), 2.75-2.69 (m, 1H), 2.57 (s, 3H), 2.25 (s, 3H), 1.98-1.84 (m, 2H).

### Example 40: N-(5-(4-(((3*S,4*R)-4-methoxypyrrolidin-3-yl)oxy)-1-methyl-1H-pyrazol-5-yl)pyrazolo[1,5-a]pyridin-2-yl)cyclopropanecarboxamide

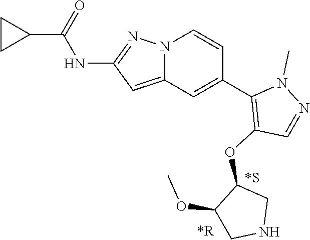 [SMILES: CO[C@@H]1CNC[C@@H]1Oc1cnn(C)c1-c1ccn2nc(NC(=O)C3CC3)cc2c1]

The title compound (19 mg, 9%) was isolated from the SFC separation found in Example 5. MS (ESI): mass calcd. for C₂₀H₂₄N₆O₃, 396.2. m/z found, 397.2 [M+H]⁺. ¹H NMR (500 MHz, Methanol-d4) δ 8.47-8.41 (m, 1H), 7.71-7.67 (m, 1H), 7.46 (s, 1H), 7.02 (dd, J=7.2, 1.9 Hz, 1H), 6.91 (s, 1H), 4.62-4.56 (m, 1H), 3.94-3.90 (m, 1H), 3.89 (s, 3H), 3.33 (s, 3H), 3.17-3.11 (m, 1H), 3.08 (dd, J=11.5, 6.1 Hz, 1H), 3.02 (dd, J=12.3, 4.1 Hz, 1H), 2.92 (dd, J=11.4, 6.4 Hz, 1H), 1.91-1.81 (m, 1H), 1.03-0.96 (m, 2H), 0.92-0.88 (m, 2H).

### Example 41: trans-N-(5-(4-(((2*R,3*S)-1,2-dimethylazetidin-3-yl)methoxy)-1-methyl-1H-pyrazol-5-yl)pyrazolo[1,5-a]pyridin-2-yl)cyclopropanecarboxamide

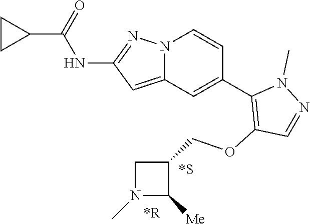 [SMILES: C[C@@H]1[C@@H](COc2cnn(C)c2-c2ccn3nc(NC(=O)C4CC4)cc3c2)CN1C]

The title compound (15 mg, 4 steps, 4%) was prepared as described in Example 6 Steps A-C using rac-tert-butyl 2-methyl-3-((tosyloxy)methyl)azetidine-1-carboxylate in place of (R)-tert-butyl 2-((tosyloxy)methyl)morpholine-4-carboxylate in step B followed by chiral separation using SFC over AD 250 mm×30 mm, 5 μm (eluent: 35% to 35% (v/v) supercritical CO₂ in MeOH and H₂O with 0.1% NH₃). The second eluting fraction was assigned as 2*R,3*S stereochemistry. MS (ESI): mass calcd. for C₂₁H₂₆N₆O₂, 394.2. m/z found, 395.2 [M+H]⁺. ¹HNMR (400 MHz, CD₃OD) δ 8.49 (d, J=6.8 Hz, 1H), 7.67 (s, 1H), 7.45 (s, 1H), 6.96 (dd, J=1.2, 7.2 Hz, 1H), 6.92 (s, 1H), 4.09-4.00 (m, 2H), 3.91-3.78 (m, 5H), 3.42 (t, J=8.8 Hz, 1H), 2.80-2.70 (m, 1H), 2.58 (s, 3H), 1.92-1.84 (m, 1H), 1.38 (d, J=6.8 Hz, 3H), 1.03-0.97 (m, 2H), 0.94-0.88 (m, 2H).

### Example 42: 5-[4-[[(2R)-azetidin-2-yl]methoxy]-2-methyl-pyrazol-3-yl]-N-(2,6-dimethylpyrimidin-4-yl)pyrazolo[1,5-a]pyridin-2-amine

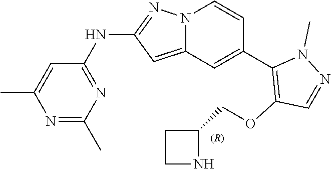

The title compound (200 mg, 2 steps, 79%) was prepared as described in Example 2 Steps A-B. MS (ESI): mass calcd. for C₂₁H₂₄N₈O, 404.2. m/z found, 405.2 [M+H]⁺. ¹H NMR (500 MHz, Chloroform-d) δ 8.49-8.43 (m, 1H), 8.15 (s, 1H), 7.48 (t, J=1.9 Hz, 1H), 7.37 (d, J=1.6 Hz, 1H), 7.03 (d, J=3.2 Hz, 1H), 6.85 (dt, J=7.2, 1.8 Hz, 1H), 6.62 (s, 1H), 4.25 (qt, J=6.8, 4.3, 3.5 Hz, 1H), 4.09-3.97 (m, 2H), 3.91 (d, J=1.5 Hz, 3H), 3.71-3.63 (m, 1H), 3.53-3.43 (m, 1H), 3.27 (s, 1H), 2.61 (d, J=1.5 Hz, 3H), 2.44 (d, J=2.2 Hz, 3H), 2.41-2.31 (m, 1H), 2.27-2.16 (m, 1H).

### Example 43: exo-N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide

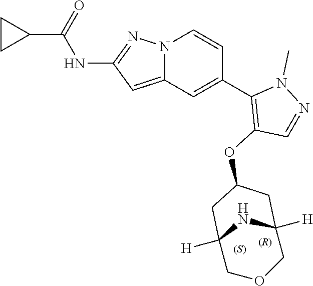

The title compound (27 mg, 9.5%) was prepared as described in Example 3 using endo-tert-butyl-(1R,5S,7s)-7-hydroxy-3-oxa-9-azabicyclo[3.3.1]nonane-9-carboxylate in place of tert-butyl (S)-2-(hydroxymethyl)-3,3-dimethylazetidine-1-carboxylate. MS (ESI): mass calcd. for C₂₂H₂₆N₆O₃, 422.2. m/z found, 423.1 [M+H]⁺. ¹H NMR (500 MHz, DMSO-d6) δ 11.00 (s, 1H), 8.53 (d, J=7.2 Hz, 1H), 7.61 (d, J=1.9 Hz, 1H), 7.32 (s, 1H), 6.89 (dd, J=7.2, 2.0 Hz, 1H), 6.82 (s, 1H), 4.85 (tt, J=11.0, 6.0 Hz, 1H), 3.76 (s, 3H), 3.61-3.47 (m, 4H), 2.82 (d, J=3.6 Hz, 2H), 2.08 (dd, J=12.2, 6.0 Hz, 2H), 1.92-1.82 (m, 1H), 1.55 (td, J=11.4, 4.2 Hz, 2H), 0.76 (dtd, J=10.4, 8.2, 5.2 Hz, 4H).

### Example 44: N-[5-[2-methyl-4-[(3-phenylazetidin-3-yl)methoxy]pyrazol-3-yl]pyrazolo[1,5-a]pyridin-2-yl]cyclopropanecarboxamide

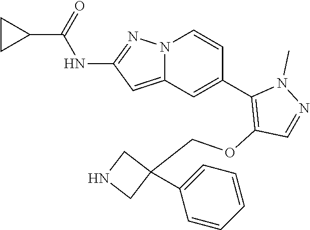 [SMILES: Cn1ncc(OCC2(c3ccccc3)CNC2)c1-c1ccn2nc(NC(=O)C3CC3)cc2c1]

The title compound (84 mg, 3 steps, 50%) was prepared as described in Example 6 using tert-butyl 3-(hydroxymethyl)-3-phenylazetidine-1-carboxylate in place of (R)-tert-butyl 2-(hydroxymethyl)morpholine-4-carboxylate in step A and trifluoracetic acid in place of HCl in step C. MS (ESI): mass calcd. for C₂₅H₂₆N₆O₂, 442.2. m/z found, 443.2 [M+H]⁺. ¹H NMR (400 MHz, DMSO-d6) δ 11.08 (s, 1H), 8.51 (dt, J=7.2, 1.0 Hz, 1H), 7.53 (dd, J=2.0, 0.9 Hz, 1H), 7.40 (s, 1H), 7.34-7.26 (m, 2H), 7.24-7.18 (m, 1H), 7.18-7.11 (m, 2H), 6.91-6.85 (m, 1H), 6.80 (dd, J=7.2, 2.0 Hz, 1H), 4.30 (s, 2H), 3.84 (s, 3H), 3.74 (d, J=7.8 Hz, 2H), 3.58 (d, J=7.9 Hz, 2H), 3.30 (s, 1H), 1.98-1.89 (m, 1H), 0.91-0.75 (m, 4H).

### Example 45: 5-[4-[[(2R)-azetidin-2-yl]methoxy]-2-methyl-pyrazol-3-yl]-N-(6-methylpyrazin-2-yl)pyrazolo[1,5-a]pyridin-2-amine

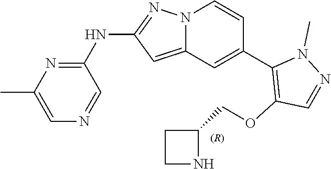

The title compound (236 mg, 2 steps, 96%) was prepared as described in Example 7 using 2-bromo-6-methylpyrazine in place of 2-chloro-6-(trifluoromethyl)pyrazine and Intermediate 6 in place of Intermediate 7 followed by Step C in Example 5. MS (ESI): mass calcd. for C₂₀H₂₂N₈O, 390.2. m/z found, 292.2 [M+H]⁺. ¹H NMR (500 MHz, Chloroform-d) δ 8.44 (s, 1H), 8.30 (dt, J=7.2, 0.9 Hz, 1H), 7.89 (s, 1H), 7.77 (s, 1H), 7.39 (dd, J=1.9, 0.8 Hz, 1H), 7.29 (s, 1H), 7.28 (d, J=4.0 Hz, OH), 6.75 (dd, J=7.1, 1.9 Hz, 1H), 6.67 (d, J=0.7 Hz, 1H), 4.15 (s, 1H), 3.99-3.89 (m, 2H), 3.83 (s, 3H), 3.59 (d, J=8.3 Hz, 1H), 3.41 (s, 1H), 2.65 (s, 2H), 2.42 (s, 3H), 2.18-2.07 (m, 1H).

### Example 46: 5-[2-methyl-4-[[(2R)-1-methylazetidin-2-yl]methoxy]pyrazol-3-yl]-N-(6-methylpyrazin-2-yl)pyrazolo[1,5-a]pyridin-2-amine

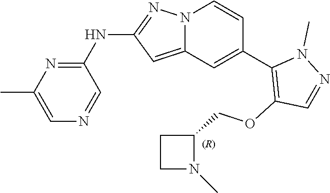

The title compound (148 mg, 3 steps, 69% yield) was prepared as described Example 8 where 5-[4-[[(2R)-azetidin-2-yl]methoxy]-2-methyl-pyrazol-3-yl]-N-(6-methylpyrazin-2-yl)pyrazolo[1,5-a]pyridin-2-amine (200 mg, 0.51 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₁H₂₄N₈O, 404.5. m/z found, 405.2 [M+H]⁺. ¹H NMR (500 MHz, Chloroform-d) δ 8.42 (d, J=18.0 Hz, 2H), 8.37-8.31 (m, 1H), 7.88 (s, 1H), 7.39 (dd, J=2.0, 0.9 Hz, 1H), 7.27 (s, 1H), 6.75 (dd, J=7.2, 1.9 Hz, 1H), 6.71-6.67 (m, 1H), 3.93-3.85 (m, 2H), 3.83 (s, 3H), 3.40-3.30 (m, 1H), 3.28-3.21 (m, 1H), 2.76-2.69 (m, 1H), 2.41 (s, 3H), 2.25 (s, 3H), 1.97-1.86 (m, 2H).

### Example 47: N-[5-[4-[[(2S)-1-ethylazetidin-2-yl]methoxy]-2-methyl-pyrazol-3-yl]pyrazolo[1,5-a]pyridin-2-yl]cyclopropanecarboxamide

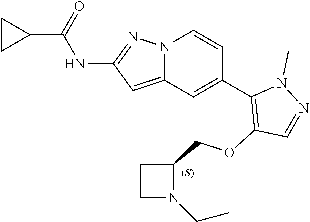

The title compound (33 mg, 31% yield) was prepared as described Example 8 step B where (S)—N-(5-(4-(azetidin-2-ylmethoxy)-1-methyl-1H-pyrazol-5-yl)pyrazolo[1,5-a]pyridin-2-yl)cyclopropanecarboxamide (Example 9, Step C, 100 mg, 0.21 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide and acetaldehyde (22 mg, 0.49 mmol) was used instead of paraformaldehyde. MS (ESI): mass calcd. for C₂₁H₂₆N₆O₂, 394.476. m/z found, 395.1 [M+H]⁺. ¹H NMR (400 MHz, CDCl₃) δ 8.43 (s, 1H), 8.29 (d, J=6.8 Hz, 1H), 7.49 (d, J=0.8 Hz, 1H), 7.33 (s, 1H), 6.99 (s, 1H), 6.84 (dd, J=1.6, 7.2 Hz, 1H), 4.05-3.92 (m, 2H), 3.90 (s, 3H), 3.45-3.33 (m, 2H), 2.81-2.73 (m, 1H), 2.71-2.61 (m, 1H), 2.40-2.30 (m, 1H), 2.09-1.92 (m, 2H), 1.62-1.53 (m, 1H), 1.18-1.12 (m, 2H), 0.98-0.87 (m, 5H).

### Example 48: N-[5-[4-[[(2S)-1-isopropylazetidin-2-yl]methoxy]-2-methyl-pyrazol-3-yl]pyrazolo[1,5-a]pyridin-2-yl]cyclopropanecarboxamide

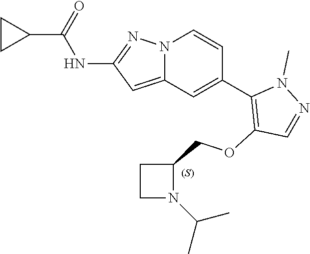

The title compound (40 mg, 36% yield) was prepared as described in Example 8 Step B where (S)—N-(5-(4-(azetidin-2-ylmethoxy)-1-methyl-1H-pyrazol-5-yl)pyrazolo[1,5-a]pyridin-2-yl)cyclopropanecarboxamide (100 mg, 0.21 mmol) was used instead of N-(5-(4-(azetidin-3-ylmethoxy)-1-methyl-1H-pyrazol-5-yl)pyrazolo[1,5-a]pyridin-2-yl)cyclopropanecarboxamide and acetone (29 mg, 0.49 mmol) was used instead of paraformaldehyde. MS (ESI): mass calcd. for C₂₂H₂₈N₆O₂, 408.503. m/z found, 409.1 [M+H]⁺. ¹H NMR (400 MHz, CDCl₃) δ 8.35 (s, 1H), 8.29 (d, J=7.2 Hz, 1H), 7.48 (d, J=1.2 Hz, 1H), 7.33 (s, 1H), 7.00 (s, 1H), 6.83 (dd, J=2.0, 7.2 Hz, 1H), 4.09-4.02 (m, 1H), 3.95-3.90 (m, 1H), 3.89 (s, 3H), 3.48-3.35 (m, 2H), 2.89-2.80 (m, 1H), 2.45-2.35 (m, 1H), 2.10-1.89 (m, 2H), 1.62-1.53 (m, 1H), 1.18-1.12 (m, 2H), 0.97-0.88 (m, 8H).

### Example 49: 5-[2-methyl-4-[[(2R)-1-methylazetidin-2-yl]methoxy]pyrazol-3-yl]-N-(5-methylpyrazin-2-yl)pyrazolo[1,5-a]pyridin-2-amine

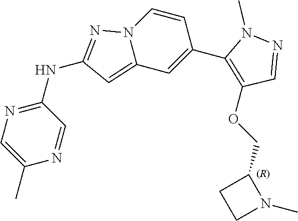

The title compound (46 mg, 3 steps, 21%) was prepared as described in Example 2 where 2-chloro-5-methylpyrazine was used instead of 4-chloro-2,6-dimethylpyrimidine. MS (ESI): mass calcd. for C₂₁H₂₄N₈O, 404.476. m/z found, 405.20 [M+H]⁺. ¹H NMR (500 MHz, Chloroform-d) δ 8.55 (d, J=1.5 Hz, 1H), 8.31 (dt, J=7.2, 0.9 Hz, 1H), 8.17 (s, 1H), 8.01 (dd, J=1.5, 0.7 Hz, 1H), 7.37 (dd, J=1.9, 0.9 Hz, 1H), 7.27 (s, 1H), 6.74 (dd, J=7.1, 1.9 Hz, 1H), 6.60 (d, J=0.8 Hz, 1H), 3.94-3.86 (m, 2H), 3.83 (s, 3H), 3.36-3.31 (m, 1H), 3.27-3.20 (m, 1H), 2.76-2.69 (m, 1H), 2.41 (s, 3H), 2.25 (s, 3H), 1.98-1.84 (m, 2H).

### Example 50: N-(6-cyclopropylpyridazin-3-yl)-5-[2-methyl-4-[[(2R)-1-methylazetidin-2-yl]methoxy]pyrazol-3-yl]pyrazolo[1,5-a]pyridin-2-amine

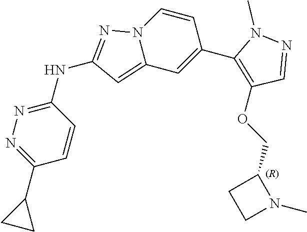

The title compound (68 mg, 3 steps, 14%) was prepared as described in Example 2 where 3-chloro-6-cyclopropylpyridazine was used instead of 4-chloro-2,6-dimethylpyrimidine. MS (ESI): mass calcd. for C₂₃H₂₆N₈O, 430.513. m/z found, 431.20 [M+H]⁺. ¹H NMR (500 MHz, Chloroform-d) δ 10.00 (s, 1H), 8.42 (d, J=7.2 Hz, 1H), 7.99 (d, J=9.2 Hz, 1H), 7.47-7.42 (m, 1H), 7.35 (s, 1H), 7.20 (d, J=9.2 Hz, 1H), 6.82-6.76 (m, 2H), 4.02-3.93 (m, 2H), 3.91 (s, 3H), 3.41 (td, J=7.3, 2.9 Hz, 1H), 3.36-3.29 (m, 1H), 2.85-2.77 (m, 1H), 2.33 (s, 3H), 2.17-2.09 (m, 1H), 2.06-1.92 (m, 2H), 1.18-1.09 (m, 2H), 1.11-1.04 (m, 2H).

### Example 51: (rac)-N-[5-[2-methyl-4-[(5-methyl-5-azaspiro[2.3]hexan-6-yl)methoxy]pyrazol-3-yl]pyrazolo[1,5-a]pyridin-2-yl]cyclopropanecarboxamide

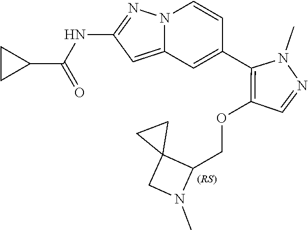 [SMILES: CN1CC2(CC2)C1COc1cnn(C)c1-c1ccn2nc(NC(=O)C3CC3)cc2c1]

The title compound (16 mg, 3 steps, 34%) was prepared as described in Example 3 where rac-tert-butyl 4-(hydroxymethyl)-5-azaspiro[2.3]hexane-5-carboxylate was used instead of tert-butyl (S)-2-(hydroxymethyl)-3,3-dimethylazetidine-1-carboxylate. MS (ESI): mass calcd. for C₂₂H₂₆N₆O₂, 406.487. m/z found, 407.2 [M+H]⁺. ¹H NMR (500 MHz, DMSO-d6) δ 11.07 (s, 1H), 8.59 (dt, J=7.2, 1.0 Hz, 1H), 7.63 (dd, J=1.9, 0.9 Hz, 1H), 7.45 (s, 1H), 6.91 (dd, J=7.2, 2.0 Hz, 1H), 6.86 (s, 1H), 3.93 (dd, J=9.7, 6.4 Hz, 1H), 3.85 (s, 3H), 3.80 (dd, J=9.6, 5.9 Hz, 1H), 3.39 (t, J=6.1 Hz, 1H), 3.28 (d, J=6.7 Hz, 1H), 3.05 (d, J=6.8 Hz, 1H), 2.30 (s, 3H), 1.98-1.87 (m, 1H), 0.89-0.76 (m, 4H), 0.66-0.57 (m, 1H), 0.44-0.37 (m, 1H), 0.36-0.28 (m, 2H).

### Example 52: N-[5-[4-[[(2R)-1-ethylazetidin-2-yl]methoxy]-2-methyl-pyrazol-3-yl]pyrazolo[1,5-a]pyridin-2-yl]cyclopropanecarboxamide

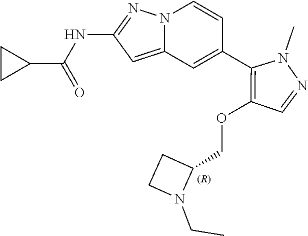

The title compound (19.7 mg, 24%) was prepared as described in Example 2 where (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) was used instead of (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine and acetaldehyde was used instead of paraformaldehyde. MS (ESI): mass calcd. for C₂₁H₂₆N₆O₂, 394.476. m/z found, 395.1 [M+H]⁺. ¹H NMR (400 MHz, CDCl₃) δ 8.43 (s, 1H), 8.29 (d, J=7.6 Hz, 1H), 7.49 (s, 1H), 7.33 (s, 1H), 6.99 (s, 1H), 6.85 (dd, J=1.2, 7.2 Hz, 1H), 4.06-3.92 (m, 2H), 3.90 (s, 3H), 3.46-3.34 (m, 2H), 2.83-2.74 (m, 1H), 2.72-2.61 (m, 1H), 2.42-2.31 (m, 1H), 2.09-1.93 (m, 2H), 1.62-1.53 (m, 1H), 1.19-1.12 (m, 2H), 0.97-0.89 (m, 5H).

### Example 53: N-[5-[4-[[(2R)-1-isopropylazetidin-2-yl]methoxy]-2-methyl-pyrazol-3-yl]pyrazolo[1,5-a]pyridin-2-yl]cyclopropanecarboxamide

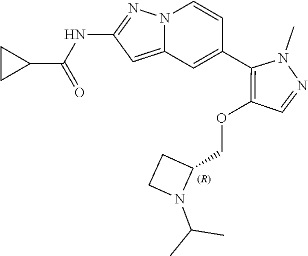

The title compound (30.8 mg, 3 steps, 29%) was prepared as described in Example 2 where (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) was used instead of (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine and propan-2-one was used instead of paraformaldehyde. MS (ESI): mass calcd. for C₂₂H₂₈N₆O₂, 408.503. m/z found, 409.2 [M+H]⁺. ¹H NMR (400 MHz, CDCl₃) δ 8.38 (s, 1H), 8.29 (d, J=7.2 Hz, 1H), 7.48 (s, 1H), 7.33 (s, 1H), 7.00 (s, 1H), 6.83 (dd, J=1.6, 7.2 Hz, 1H), 4.10-4.03 (m, 1H), 3.95-3.91 (m, 1H), 3.89 (s, 3H), 3.49-3.36 (m, 2H), 2.91-2.81 (m, 1H), 2.46-2.37 (m, 1H), 2.10-2.01 (m, 1H), 2.00-1.91 (m, 1H), 1.62-1.53 (m 1H), 1.18-1.12 (m, 2H), 0.97-0.87 (m, 8H)

### Example 54: (rac)-N-[5-[4-[(1,2-dimethylazetidin-2-yl)methoxy]-2-methyl-pyrazol-3-yl]pyrazolo[1,5-a]pyridin-2-yl]cyclopropanecarboxamide

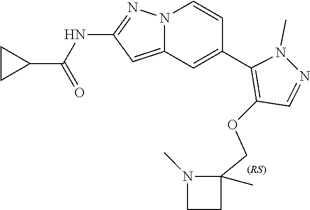 [SMILES: CN1CCC1(C)COc1cnn(C)c1-c1ccn2nc(NC(=O)C3CC3)cc2c1]

The title compound (12 mg, 3 steps, 3%) was prepared as described in Example 3 where tert-butyl 2-(hydroxymethyl)-2-methylazetidine-1-carboxylate was used instead of tert-butyl (S)-2-(hydroxymethyl)-3,3-dimethylazetidine-1-carboxylate. MS (ESI): mass calcd. for C₂₁H₂₆N₆O₂, 394.476. m/z found, 395.2 [M+H]⁺. ¹H NMR (400 MHz, Methanol-d4) δ 8.50 (dt, J=7.2, 0.9 Hz, 1H), 7.70 (dd, J=2.0, 1.0 Hz, 1H), 7.49 (s, 1H), 7.01 (dd, J=7.2, 1.9 Hz, 1H), 6.94 (s, 1H), 4.02 (d, J=9.6 Hz, 1H), 3.94 (d, J=3.9 Hz, 4H), 3.34-3.27 (m, 1H), 3.14 (dt, J=8.6, 7.4 Hz, 1H), 2.25 (s, 3H), 2.23-2.13 (m, 1H), 1.97-1.82 (m, 2H), 1.35 (s, 3H), 1.04 (dt, J=4.6, 3.1 Hz, 2H), 0.99-0.90 (m, 2H).

### Example 55: 5-[2-methyl-4-[[(2R)-1-methylazetidin-2-yl]methoxy]pyrazol-3-yl]-N-(6-methylpyrimidin-4-yl)pyrazolo[1,5-a]pyridin-2-amine

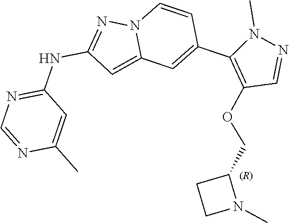

The title compound (146 mg, 3 steps, 61%) was prepared as described in Example 2 where 4-bromo-6-methylpyrimidine was used instead of 4-chloro-2,6-dimethylpyrimidine. MS (ESI): mass calcd. for C₂₁H₂₄N₈O, 404.476. m/z found, 405.20 [M+H]⁺. ¹H NMR (500 MHz, Chloroform-d) δ 9.02 (s, 1H), 8.68 (s, 1H), 8.39-8.34 (m, 1H), 7.40 (s, 1H), 7.28 (s, 1H), 7.20 (d, J=7.7 Hz, 1H), 6.82-6.77 (m, 1H), 6.51 (s, 1H), 3.93-3.87 (m, 3H), 3.84 (s, 3H), 3.37-3.31 (m, 1H), 3.29-3.21 (m, 1H), 2.73 (q, J=8.1 Hz, 1H), 2.40 (s, 4H), 2.25 (s, 3H).

### Example 56: N-[5-[4-[[(2R)-1-acetylazetidin-2-yl]methoxy]-2-methyl-pyrazol-3-yl]pyrazolo[1,5-a]pyridin-2-yl]cyclopropanecarboxamide

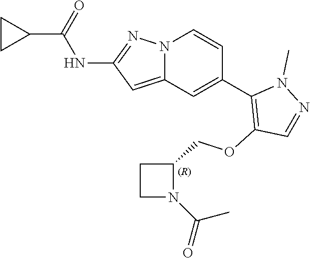

To a mixture of (R)—N-(5-(4-(azetidin-2-ylmethoxy)-1-methyl-1H-pyrazol-5-yl)pyrazolo[1,5-a]pyridin-2-yl)cyclopropanecarboxamide (Example 346, 100 mg, 0.208 mmol) and DCM (1 mL) was added TEA (42.1 mg, 0.416 mmol) and AcCl (16.3 mg, 0.208 mmol) at 25° C. The mixture was stirred at 25° C. for 16 hours. The reaction mixture was filtered. The filtrate was purified by preparative HPLC using a YMC-Triart Prep C18 250×50 mm×10 μm column (eluent: 25% to 55% (v/v) CH₃CN and H₂O with 0.04% NH₃). The material was suspended in water (10 mL), the mixture frozen using dry ice/ethanol, and then lyophilized to dryness to afford the title compound (16.4 mg, 19.3%) as a white solid. MS (ESI): mass calcd. for C₂₁H₂₄N₆O₃, 408.46. m/z found, 409.1 [M+H]⁺. ¹H NMR (400 MHz, DMSO-d6) δ 11.12 (s, 1H), 8.69-8.53 (m, 1H), 7.76-7.62 (m, 1H), 7.56-7.37 (m, 1H), 7.03-6.93 (m, 1H), 6.92-6.85 (m, 1H), 4.71-4.61; 4.49-4.38 (m, 1H), 4.34-4.26; 4.09-4.01 (m, 1H), 4.24-4.12; 4.00-3.89 (m, 2H), 3.89-3.84 (m, 3H), 3.77-3.60 (m, 1H), 2.39-2.21 (m, 1H), 2.19-2.08; 2.04-1.86 (m, 2H), 1.79-1.59 (m, 3H), 0.91-0.73 (m, 4H); ¹H NMR (400 MHz, DMSO-d6, t=80° C.): δ 10.80-10.65 (m, 1H), 8.51 (d, J=7.2 Hz, 1H), 7.64 (s, 1H), 7.41 (s, 1H), 6.92 (d, J=6.8 Hz, 1H), 6.87 (s, 1H), 4.60-4.40 (m, 1H), 4.29-4.17 (m, 1H), 4.16-4.05 (m, 1H), 3.95-3.72 (m, 4H), 3.07-3.06 (m, 1H), 2.37-2.20 (m, 1H), 2.17-2.00 (m, 1H), 1.99-1.90 (m, 1H), 1.68 (s, 3H), 0.89-0.74 (m, 4H).

### Example 57: 5-[2-methyl-4-[[(2R)-1-methylazetidin-2-yl]methoxy]pyrazol-3-yl]-N-(2,5,6-trimethylpyrimidin-4-yl)pyrazolo[1,5-a]pyridin-2-amine

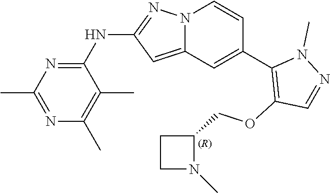

The title compound (73 mg, 3 steps, 64%) was prepared as described in Example 2 where 4-chloro-2,5,6-trimethylpyrimidine was used instead of 4-chloro-2,6-dimethylpyrimidine. MS (ESI): mass calcd. for C₂₃H₂₈N₈O, 432.529. m/z found, 433.20 [M+H]⁺. ¹H NMR (500 MHz, Chloroform-d) δ 8.21 (dt, J=7.2, 1.0 Hz, 1H), 7.44 (dd, J=2.0, 1.0 Hz, 1H), 7.29-7.23 (m, 2H), 7.21 (s, 1H), 6.77 (dd, J=7.2, 1.9 Hz, 1H), 3.95-3.86 (m, 2H), 3.84 (s, 3H), 3.37-3.30 (m, 1H), 3.28-3.22 (m, 1H), 2.77-2.70 (m, 1H), 2.55 (s, 3H), 2.37 (s, 3H), 2.25 (s, 3H), 2.11 (s, 3H), 1.99-1.84 (m, 2H).

### Example 58: (*R)—N-(5-(1-methyl-4-((2-methyl-7-oxa-2-azaspiro[3.5]nonan-1-yl)methoxy)-1H-pyrazol-5-yl)pyrazolo[1,5-a]pyridin-2-yl)cyclopropanecarboxamide

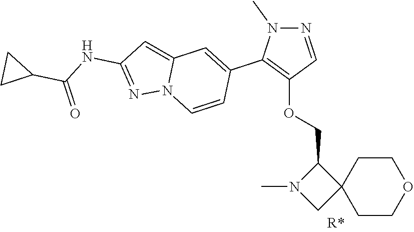 [SMILES: CN1CC2(CCOCC2)[C@@H]1COc1cnn(C)c1-c1ccn2nc(NC(=O)C3CC3)cc2c1]

The title compound (25 mg, 3 steps, 17%) was prepared as described in Example 3 where tert-butyl 1-(hydroxymethyl)-7-oxa-2-azaspiro[3.5]nonane-2-carboxylate was used instead of tert-butyl (S)-2-(hydroxymethyl)-3,3-dimethylazetidine-1-carboxylate. The crude product obtained from Step C was purified via preparative supercritical fluid chromatography (SFC) using OJ-H (2×25 cm) as stationary phase; isocratic 15% MeOH/CO₂, 100 bar. MS (ESI): mass calcd. for C₂₄H₃₀N₆O₃, 450.54. m/z found, 451.3 [M+H]⁺. ¹H NMR (400 MHz, DMSO-d6) δ 11.07 (s, 1H), 8.59 (dt, J=7.1, 0.9 Hz, 1H), 7.66 (dd, J=2.0, 0.9 Hz, 1H), 7.48 (s, 1H), 6.93 (dd, J=7.2, 2.0 Hz, 1H), 6.86 (s, 1H), 4.02 (dd, J=9.9, 6.7 Hz, 1H), 3.92 (dd, J=9.9, 6.3 Hz, 1H), 3.85 (s, 3H), 3.65 (dt, J=11.5, 3.9 Hz, 1H), 3.57 (dt, J=11.4, 3.9 Hz, 1H), 3.39-3.33 (m, 1H), 3.19 (td, J=11.0, 2.8 Hz, 1H), 2.91 (t, J=6.4 Hz, 1H), 2.54 (d, J=6.6 Hz, 1H), 2.23 (s, 3H), 1.98-1.86 (m, 1H), 1.67 (t, J=5.4 Hz, 2H), 1.65-1.54 (m, 1H), 1.52-1.43 (m, 1H), 0.89-0.73 (m, 4H).

### Example 59: (*S)—N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide

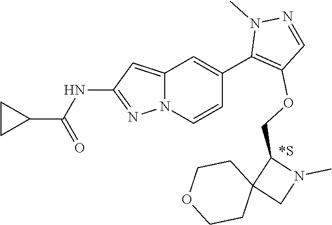 [SMILES: CN1CC2(CCOCC2)[C@H]1COc1cnn(C)c1-c1ccn2nc(NC(=O)C3CC3)cc2c1]

The title compound (25 mg, 3 steps, 17%) was prepared as described in Example 3 where tert-butyl 1-(hydroxymethyl)-7-oxa-2-azaspiro[3.5]nonane-2-carboxylate was used instead of tert-butyl (S)-2-(hydroxymethyl)-3,3-dimethylazetidine-1-carboxylate. The crude product obtained from Step C was purified via preparative SFC using OJ-H (2×25 cm) as stationary phase; isocratic 15% MeOH/CO₂,100 bar. MS (ESI): mass calcd. for C₂₄H₃₀N₆O₃, 450.54. m/z found, 451.3 [M+H]⁺. ¹H NMR (400 MHz, DMSO-d6) δ 11.07 (s, 1H), 8.60 (dt, J=7.1, 1.0 Hz, 1H), 7.67 (dd, J=2.0, 0.9 Hz, 1H), 7.49 (s, 1H), 6.94 (dd, J=7.2, 2.0 Hz, 1H), 6.87 (s, 1H), 4.03 (dd, J=9.9, 6.6 Hz, 1H), 3.93 (dd, J=9.9, 6.3 Hz, 1H), 3.86 (s, 3H), 3.66 (dt, J=11.4, 3.9 Hz, 1H), 3.58 (dt, J=11.4, 4.0 Hz, 1H), 3.41-3.33 (m, 1H), 3.30-3.27 (m, 1H), 3.20 (td, J=11.0, 2.7 Hz, 1H), 2.92 (t, J=6.5 Hz, 1H), 2.55 (d, J=6.6 Hz, 1H), 2.24 (s, 3H), 1.99-1.85 (m, 1H), 1.72-1.65 (m, 2H), 1.65-1.56 (m, 1H), 1.53-1.44 (m, 1H), 0.83 (m, J=7.8, 2.9 Hz, 4H).

### Example 60: rac-N-[5-[4-[(3,3-difluoro-1-methyl-azetidin-2-yl)methoxy]-2-methyl-pyrazol-3-yl]pyrazolo[1,5-a]pyridin-2-yl]cyclopropanecarboxamide

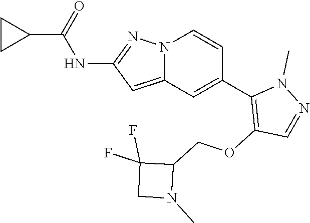 [SMILES: CN1CC(F)(F)C1COc1cnn(C)c1-c1ccn2nc(NC(=O)C3CC3)cc2c1]

The title compound (18 mg, 2 steps, 11%) was prepared as described in Example 3 where tert-butyl (R)-3,3-difluoro-2-(hydroxymethyl)azetidine-1-carboxylate was used instead of tert-butyl (S)-2-(hydroxymethyl)-3,3-dimethylazetidine-1-carboxylate. MS (ESI): mass calcd. for C₂₀H₂₂F₂N₆O₂, 416.43. m/z found, 417.20 [M+H]⁺. ¹H NMR (400 MHz, Methanol-d4) δ 8.35 (dt, J=7.2, 0.9 Hz, 1H), 7.53 (dd, J=2.0, 1.0 Hz, 1H), 7.37 (s, 1H), 6.84 (dd, J=7.2, 2.0 Hz, 1H), 6.80 (s, 1H), 4.11 (dd, J=10.2, 6.2 Hz, 1H), 4.04-3.94 (m, 1H), 3.79 (s, 3H), 3.76-3.55 (m, 2H), 2.35 (dd, J=1.9, 1.0 Hz, 3H), 1.77 (tt, J=8.1, 4.6 Hz, 1H), 0.90 (dt, J=4.5, 3.1 Hz, 2H), 0.86-0.71 (m, 2H).

### Example 61: N-(4,6-dimethylpyridazin-3-yl)-5-[2-methyl-4-[[(2R)-1-methylazetidin-2-yl]methoxy]pyrazol-3-yl]pyrazolo[1,5-a]pyridin-2-amine

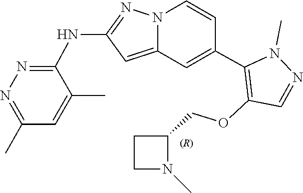

The title compound (20 mg, 3 steps, 8%) was prepared as described in Example 2 where 3-chloro-4,6-dimethyl-pyridazine was used instead 4-chloro-2,6-dimethylpyrimidine. MS (ESI): mass calcd. for C₂₂H₂₆N₈O, 418.502. m/z found, 419.20 [M+H]⁺. ¹H NMR (500 MHz, Chloroform-d) δ 8.22 (d, J=7.1 Hz, 1H), 7.44-7.40 (m, 1H), 7.35 (s, 1H), 7.27 (s, 1H), 6.97 (d, J=10.7 Hz, 2H), 6.77-6.71 (m, 1H), 3.95-3.85 (m, 2H), 3.83 (s, 3H), 3.34 (td, J=7.4, 2.7 Hz, 1H), 3.25 (p, J=7.3 Hz, 1H), 2.79-2.70 (m, 1H), 2.52 (s, 3H), 2.25 (d, J=14.8 Hz, 6H), 2.00-1.85 (m, 2H).

### Example 62: N-(2,6-dimethylpyrimidin-4-yl)-5-[4-[[(2R)-1-ethylazetidin-2-yl]methoxy]-2-methyl-pyrazol-3-yl]pyrazolo[1,5-a]pyridin-2-amine

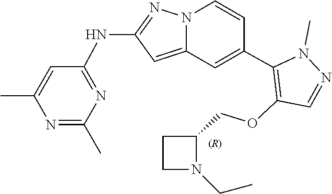

The title compound (104 mg, 62%) was prepared as described in Example 2 where acetaldehyde was used instead of H(CHO)n. MS (ESI): mass calcd. for C₂₃H₂₈N₈O, 432.529. m/z found, 433.20 [M+H]⁺. ¹H NMR (500 MHz, Chloroform-d) δ 8.76 (s, 1H), 8.72 (s, OH), 8.45 (d, J=7.2 Hz, 1H), 7.39 (d, J=1.9 Hz, 1H), 7.27 (s, 1H), 6.92 (s, 1H), 6.77 (dd, J=7.2, 2.0 Hz, 1H), 6.54 (s, 1H), 3.94 (dd, J=9.8, 6.5 Hz, 1H), 3.88 (dd, J=9.8, 4.7 Hz, 1H), 3.83 (s, 3H), 3.36-3.24 (m, 2H), 3.14 (d, J=4.4 Hz, OH), 2.83 (s, OH), 2.72-2.54 (m, 2H), 2.53 (s, 3H), 2.35 (s, 3H), 2.30-2.22 (m, 1H), 2.00-1.83 (m, 2H), 0.86 (t, J=7.2 Hz, 3H).

### Example 63: 5-[2-methyl-4-[[(2R)-1-methylazetidin-2-yl]methoxy]pyrazol-3-yl]-N-[6-methyl-2-(trifluoromethyl)pyrimidin-4-yl]pyrazolo[1,5-a]pyridin-2-amine

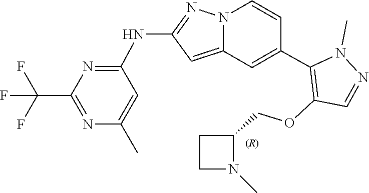

The title compound (135 mg, 3 steps, 61%) was prepared as described in Example 2 where 4-chloro-6-methyl-2-trifluoromethylpyrimidine was used instead of 4-chloro-2,6-dimethylpyrimidine. MS (ESI): mass calcd. for C₂₂H₂₃F₃N₈O, 472.473. m/z found, 473.20 [M+H]⁺. ¹H NMR (400 MHz, Chloroform-d) δ 9.78 (s, 1H), 8.64 (d, J=7.3 Hz, 1H), 7.45 (d, J=1.9 Hz, 1H), 7.33 (s, 1H), 7.24 (s, 1H), 6.88 (dd, J=7.2, 1.9 Hz, 1H), 6.59 (s, 1H), 4.00 (d, J=5.3 Hz, 2H), 3.90 (s, 3H), 3.45 (td, J=6.9, 3.6 Hz, 1H), 3.41-3.29 (m, 1H), 2.97 (s, 3H), 2.82 (td, J=8.7, 6.8 Hz, 1H), 2.49 (s, 3H), 2.03 (qd, J=8.3, 7.8, 4.3 Hz, 2H).

### Example 64: 5-[2-methyl-4-[[(2R)-1-methylazetidin-2-yl]methoxy]pyrazol-3-yl]-N-(2-methylpyrimidin-4-yl)pyrazolo[1,5-a]pyridin-2-amine

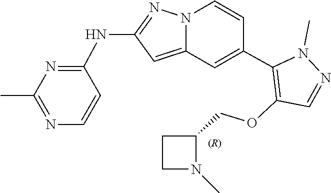

The title compound (54 mg, 3 steps, 24%) was prepared as described in Example 2 where 4-chloro-2-methylpyrimidine was used instead of 4-chloro-2,6-dimethylpyrimidine. MS (ESI): mass calcd. for C₂₁H₂₄N₈O, 404.476. m/z found, 405.20 [M+H]⁺. ¹H NMR (500 MHz, Chloroform-d) δ 8.76 (d, J=3.2 Hz, 1H), 8.42 (d, J=7.2 Hz, 1H), 8.26 (d, J=5.9 Hz, 1H), 7.42-7.38 (m, 1H), 7.27 (s, 1H), 7.05 (d, J=6.0 Hz, 1H), 6.79 (dd, J=7.2, 1.9 Hz, 1H), 6.57 (s, 1H), 3.90 (d, J=5.6 Hz, 2H), 3.84 (s, 3H), 3.34 (td, J=7.2, 3.0 Hz, 1H), 3.28-3.21 (m, 1H), 2.73 (td, J=8.3, 6.8 Hz, 1H), 2.56 (s, 3H), 2.25 (s, 3H), 1.97-1.86 (m, 2H).

### Example 65: 5-[4-[[(2R)-1-ethylazetidin-2-yl]methoxy]-2-methyl-pyrazol-3-yl]-N-(2-methylpyrimidin-4-yl)pyrazolo[1,5-a]pyridin-2-amine

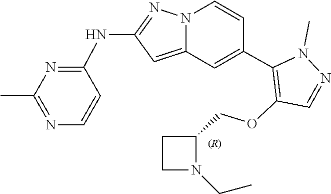

The title compound (55.8 mg, 3 steps, 23%) was prepared as described in Example 2 where 4-chloro-2-methylpyrimidine was used instead of 4-chloro-2,6-dimethylpyrimidine and acetaldehyde was used instead of H(CHO)n. MS (ESI): mass calcd. for C₂₂H₂₆N₈O, 418.502. m/z found, 419.20 [M+H]⁺. ¹H NMR (500 MHz, Chloroform-d) δ 8.35 (dt, J=7.2, 0.9 Hz, 1H), 8.29 (d, J=5.9 Hz, 1H), 7.98 (s, 1H), 7.40 (dd, J=1.9, 0.9 Hz, 1H), 7.27 (s, 1H), 7.09 (d, J=5.9 Hz, 1H), 6.78 (dd, J=7.2, 1.9 Hz, 1H), 6.55 (s, 1H), 3.94 (dd, J=9.8, 6.5 Hz, 1H), 3.89 (dd, J=9.8, 4.8 Hz, 1H), 3.84 (s, 3H), 3.37-3.25 (m, 2H), 2.72-2.66 (m, 1H), 2.65-2.56 (m, 1H), 2.57 (s, 3H), 2.32-2.24 (m, 1H), 2.01-1.84 (m, 2H), 0.87 (t, J=7.2 Hz, 3H).

### Example 66: (1R,2R)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide

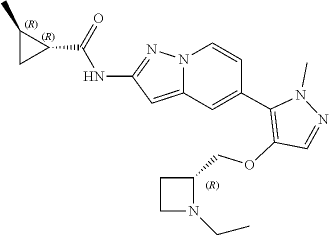

The title compound was prepared (50 mg, 3 steps, 33%) by the similar method of Example 24 by using (1R,2R)-2-methylcyclopropane-1-carboxylic acid instead of (1S,2S)-2-methylcyclopropane-1-carboxylic acid. MS (ESI): mass calcd. for C₂₂H₂₈N₆O₂, 408.503. m/z found, 409.20 [M+H]⁺. ¹H NMR (400 MHz, Chloroform-d) δ 9.14 (d, J=8.1 Hz, 1H), 8.30 (d, J=7.2 Hz, 1H), 7.49 (dd, J=1.9, 0.9 Hz, 1H), 7.33 (s, 1H), 7.01 (s, 1H), 6.84 (dd, J=7.2, 1.9 Hz, 1H), 4.01 (dd, J=9.8, 6.4 Hz, 1H), 3.94 (dd, J=9.8, 4.8 Hz, 1H), 3.89 (s, 3H), 3.47 (s, 1H), 3.45-3.30 (m, 2H), 2.80-2.72 (m, 1H), 2.70-2.61 (m, 1H), 2.40-2.29 (m, 1H), 2.09-1.90 (m, 2H), 1.59-1.48 (m, 1H), 1.36-1.20 (m, 2H), 1.12 (d, J=6.2 Hz, 3H), 0.93 (t, J=7.2 Hz, 3H).

### Example 67: (*S)—N-[5-[4-[(5-ethyl-5-azaspiro[2.3]hexan-6-yl)methoxy]-2-methyl-pyrazol-3-yl]pyrazolo[1,5-a]pyridin-2-yl]cyclopropanecarboxamide

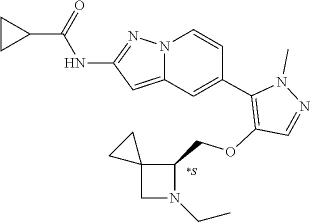

The title compound was prepared (15 mg, 38% yield) by the similar method of Example 16 by using (*S)—N-(5-(4-((5-azaspiro[2.3]hexan-4-yl)methoxy)-1-methyl-1H-pyrazol-5-yl)pyrazolo[1,5-a]pyridin-2-yl)cyclopropanecarboxamide instead of the enantiomer in Step D. MS (ESI): mass calcd. for C₂₃H₂₈N₆O₂, 420.514. m/z found, 421.2 [M+H]⁺. ¹H NMR (400 MHz, DMSO-d6) δ 11.07 (s, 1H), 8.58 (dt, J=7.2, 1.0 Hz, 1H), 7.62 (dd, J=2.0, 0.9 Hz, 1H), 7.45 (s, 1H), 6.89 (dd, J=7.2, 2.0 Hz, 1H), 6.86 (d, J=0.9 Hz, 1H), 3.94 (dd, J=9.7, 5.9 Hz, 1H), 3.84 (s, 3H), 3.81 (dd, J=9.6, 6.4 Hz, 1H), 3.42 (t, J=6.1 Hz, 1H), 3.29-3.23 (m, 2H), 3.00 (d, J=6.9 Hz, 1H), 2.69-2.55 (m, 1H), 2.40 (dq, J=11.3, 7.1 Hz, 1H), 2.00-1.86 (m, 1H), 0.89-0.77 (m, 7H), 0.70-0.57 (m, 1H), 0.46-0.36 (m, 1H), 0.36-0.26 (m, 2H).

### Example 68: 5-[4-[[(2R)-1-ethylazetidin-2-yl]methoxy]-2-methyl-pyrazol-3-yl]-N-(6-methylpyridazin-3-yl)pyrazolo[1,5-a]pyridin-2-amine

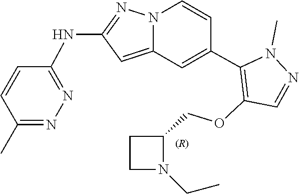

The title compound was prepared (38 mg, 3 steps, 6% yield) by the similar method of Example 2 by using 3-bromo-6-methylpyridazine and acetaldehyde instead of 4-chloro-6-methyl-2-(trifluoromethyl)pyrimidine and paraformaldehyde. MS (ESI): mass calcd. for C₂₂H₂₆N₈O, 418.502. m/z found, 419.20 [M+H]⁺. ¹H NMR (500 MHz, Chloroform-d) δ 9.54 (s, 1H), 8.41 (d, J=7.1 Hz, 1H), 8.03 (d, J=9.1 Hz, 1H), 7.46 (dd, J=1.9, 0.9 Hz, 1H), 7.35 (s, 1H), 7.29 (d, J=9.2 Hz, 1H), 6.79 (dd, J=7.2, 1.9 Hz, 1H), 6.72 (s, 1H), 4.02 (dd, J=9.8, 6.4 Hz, 1H), 3.96 (dd, J=9.8, 4.8 Hz, 1H), 3.91 (s, 3H), 3.45-3.33 (m, 2H), 2.80-2.73 (m, 1H), 2.73-2.62 (m, 1H), 2.66 (s, 3H), 2.40-2.32 (m, 1H), 2.09-1.92 (m, 2H), 0.95 (t, J=7.2 Hz, 3H).

### Example 69: (1S,2S)-2-fluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide

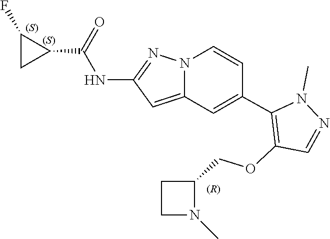

The title compound was prepared (12 mg, 12%) by the similar method of Example 26 by using (1S,2S)-2-fluorocyclopropane-1-carboxylic acid instead of 1-methyl-1H-pyrazole-4-carboxylic acid. MS (ESI): mass calcd. for C₂₀H₂₃FN₆O₂, 398.44. m/z found, 399.1 [M+H]⁺. ¹H NMR (500 MHz, Methanol-d4) δ 8.36 (dd, J=7.2, 1.0 Hz, 1H), 7.56 (dd, J=2.0, 0.9 Hz, 1H), 7.34 (s, 1H), 6.92-6.85 (m, 1H), 6.83 (d, J=0.9 Hz, 1H), 3.94-3.86 (m, 2H), 3.79 (s, 3H), 4.80 (m, 1H) 3.41-3.34 (m, 1H), 3.30-3.26 (m, 1H), 2.86-2.79 (m, 1H), 2.21 (s, 3H), 1.98-1.88 (m, 3H), 1.73-1.64 (m, 1H), 1.16-1.04 (m, 2H).

### Example 70: trans-N-[5-[2-methyl-4-[((2R)-1-methylazetidin-2-yl)methoxy]pyrazol-3-yl]pyrazolo[1,5-a]pyridin-2-yl]-2-(trifluoromethyl)cyclopropanecarboxamide

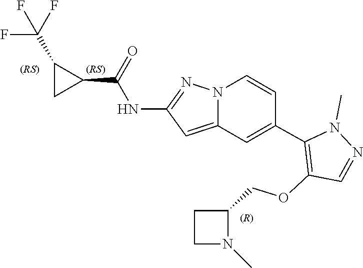

The title compound was prepared (5 mg, 4%) by the similar method of Example 26 by using trans-2-(trifluoromethyl)cyclopropane-1-carboxylic acid instead of 1-methyl-1H-pyrazole-4-carboxylic acid. MS (ESI): mass calcd. for C₂₁H₂₃F₃N₆O₂, 448.447. m/z found, 449.1 449.1 [M+H]⁺. ¹H NMR (500 MHz, Methanol-d4) δ 8.42-8.32 (m, 1H), 7.43 (s, 1H), 7.34 (s, 1H), 6.95-6.85 (m, 1H), 6.82 (d, J=0.9 Hz, 1H), 3.97-3.85 (m, 2H), 3.79 (s, 3H), 3.40-3.33 (m, 1H), 3.29-3.25 (m, 1H), 2.87-2.72 (m, 1H), 2.22 (s, 4H), 2.17-2.09 (m, 1H), 2.00-1.82 (m, 2H), 1.34-1.27 (m, 1H), 1.25-1.19 (m, 1H).

### Example 71: trans-N-[5-[2-methyl-4-[((2R)-1-methylazetidin-2-yl)methoxy]pyrazol-3-yl]pyrazolo[1,5-a]pyridin-2-yl]-2-phenyl-cyclopropanecarboxamide

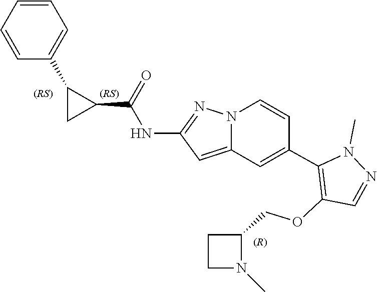

The title compound was prepared (14 mg, 12% yield) by the similar method of Example 26 by using trans-2-phenylcyclopropane-1-carboxylic acid instead of 1-methyl-1H-pyrazole-4-carboxylic acid. MS (ESI): mass calcd. for C₂₆H₂₈N₆O₂, 456.547. m/z found, 457.2 [M+H]⁺. ¹H NMR (500 MHz, Methanol-d4) δ 8.40-8.32 (m, 1H), 7.57 (dd, J=2.0, 0.9 Hz, 1H), 7.35 (s, 1H), 7.21-7.15 (m, 2H), 7.13-7.07 (m, 3H), 7.03-6.96 (m, 1H), 6.89-6.84 (m, 1H), 4.02-3.92 (m, 2H), 3.79 (s, 3H), 3.56-3.47 (m, 1H), 3.39-3.30 (m, 1H), 3.04-2.89 (m, 1H), 2.47-2.39 (m, 1H), 2.29 (s, 3H), 2.08-1.91 (m, 3H), 1.56-1.49 (m, 1H), 1.35-1.30 (m, 1H).

### Example 72: 2,2-difluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide

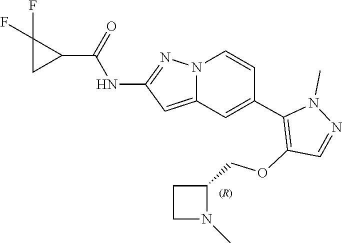

The title compound was prepared (7 mg, 7% yield) by the similar method of Example 26 by using 2,2-difluorocyclopropane-1-carboxylic acid instead of 1-methyl-1H-pyrazole-4-carboxylic acid. MS (ESI): mass calcd. for C₂₀H₂₂F₂N₆O₂, 416.43. m/z found, 417.1 [M+H]⁺. ¹H NMR (400 MHz, Methanol-d4) δ 8.40-8.32 (m, 1H), 7.57 (dd, J=2.0, 1.0 Hz, 1H), 7.34 (s, 1H), 6.93-6.81 (m, 2H), 3.99-3.86 (m, 2H), 3.79 (s, 3H), 3.42-3.34 (m, 1H), 3.32-3.26 (m, 1H), 2.85-2.63 (m, 2H), 2.21 (s, 3H), 2.07-1.99 (m, 1H), 1.99-1.87 (m, 2H), 1.83-1.73 (m, 1H).

### Example 73: 5-[4-[[(2R)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine

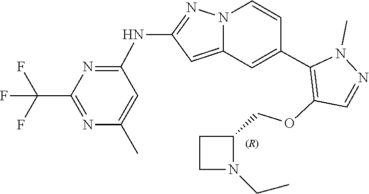

The title compound was prepared (126 mg, 65%) by the similar method of Example 2 by using acetaldehyde instead of paraldehyde and 4-chloro-6-methyl-2-trifluoromethylpyrimidine instead of 4-chloro-2,6-dimethylpyrimidine. MS (ESI): mass calcd. for C₂₃H₂₅F₃N₈O, 486.5. m/z found, 487.30 [M+H]⁺. ¹H NMR (400 MHz, Chloroform-d) δ 9.26 (s, 1H), 8.64 (d, J=7.2 Hz, 1H), 7.50 (dd, J=2.0, 0.9 Hz, 1H), 7.35 (s, 1H), 6.90 (dd, J=7.2, 1.9 Hz, 1H), 6.66-6.60 (m, 1H), 4.07-3.97 (m, 2H), 3.92 (s, 3H), 3.49-3.34 (m, 2H), 2.83-2.64 (m, 2H), 2.54 (s, 3H), 2.46-2.30 (m, 2H), 2.12-1.95 (m, 2H), 0.94 (t, J=7.2 Hz, 3H).

### Example 74: N-[5-[5-methyl-3-[(3R)-pyrrolidin-3-yl]oxy-isoxazol-4-yl]pyrazolo[1,5-a]pyridin-2-yl]cyclopropanecarboxamide

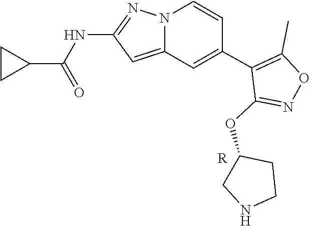

The title compound (21 mg, 2 steps, 4.3%) was prepared as described in Example 28, except (S)-tert-butyl-3-bromopyrrolidine-1-carboxylate was used instead of tert-butyl (S)-2-((tosyloxy)methyl)morpholine-4-carboxylate in Step B. MS (ESI): mass calcd. for C₁₉H₂₁N₅O₃, 367.4. m/z found, 368.2 [M+H]⁺. ¹H NMR (500 MHz, DMSO-d6) δ 11.03 (s, 1H), 8.57-8.50 (m, 1H), 7.63 (dd, J=2.0, 0.9 Hz, 1H), 6.90 (dd, J=7.2, 2.0 Hz, 1H), 6.85 (s, 1H), 5.19-5.08 (m, 1H), 3.11-2.98 (m, 2H), 2.98-2.90 (m, 1H), 2.85-2.74 (m, 1H), 2.54 (s, 1H), 2.51 (s, 3H), 2.11-1.99 (m, 1H), 1.97-1.85 (m, 2H), 0.88-0.77 (m, 4H).

### Example 75: N-[5-[5-methyl-3-[(3S)-pyrrolidin-3-yl]oxy-isoxazol-4-yl]pyrazolo[1,5-a]pyridin-2-yl]cyclopropanecarboxamide

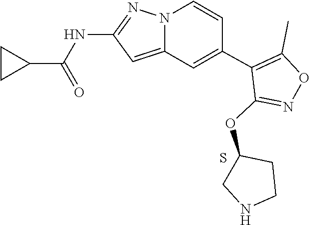

The title compound (182 mg, 3 steps, 18%) was prepared as described in Example 31, except (R)-tert-butyl-3-(tosyloxy)pyroolidine-1-carboxylate was used instead of (S)-2-((tert-butoxycarbonyl)amino)-2-phenylethyl 4-methylbenzenesulfonate in step A. MS (ESI): mass calcd. for C₁₉H₂₁N₅O₃, 367.4. m/z found, 368.1 [M+H]⁺. ¹H NMR (500 MHz, DMSO-d6) δ 11.03 (s, 1H), 8.54 (d, J=7.3 Hz, 1H), 7.63 (d, J=1.9 Hz, 1H), 6.90 (dd, J=7.3, 2.0 Hz, 1H), 6.85 (s, 1H), 5.16-5.05 (m, 1H), 3.08-2.86 (m, 3H), 2.81-2.71 (m, 1H), 2.51 (s, 3H), 2.08-1.97 (m, 1H), 1.96-1.82 (m, 2H), 0.88-0.76 (m, 4H).

### Example 76: N-[5-[3-[(3-methoxyazetidin-3-yl)methoxy]-5-methyl-isoxazol-4-yl]pyrazolo[1,5-a]pyridin-2-yl]cyclopropanecarboxamide

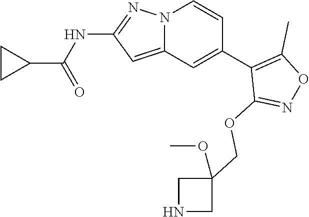 [SMILES: COC1(COc2noc(C)c2-c2ccn3nc(NC(=O)C4CC4)cc3c2)CNC1]

The title compound (98 mg, 4 steps, 24%) was prepared as described in Example 31, except tert-butyl 3-methoxy-3-((tosyloxy)methyl)azetidine-1-carboxylate was used instead of (S)-2-((tert-butoxycarbonyl)amino)-2-phenylethyl 4-methylbenzenesulfonate in step A. MS (ESI): mass calcd. for C₂₀H₂₃N₅O₄, 397.4. m/z found, 398.1 [M+H]⁺. ¹H NMR (400 MHz, DMSO-d6) δ 11.05 (s, 1H), 8.57 (d, J=7.1 Hz, 1H), 7.64 (dd, J=2.0, 0.9 Hz, 1H), 6.89 (dd, J=7.2, 2.0 Hz, 1H), 6.86-6.77 (m, 1H), 4.56 (s, 2H), 3.53 (d, J=8.4 Hz, 2H), 3.30-3.26 (m, 2H), 3.20 (s, 3H), 2.53 (s, 3H), 1.93 (d, J=12.3 Hz, 1H), 0.81 (dd, J=9.3, 3.3 Hz, 4H).

### Example 77: N-[5-[3-[(3-cyanoazetidin-3-yl)methoxy]-5-methyl-isoxazol-4-yl]pyrazolo[1,5-a]pyridin-2-yl]cyclopropanecarboxamide

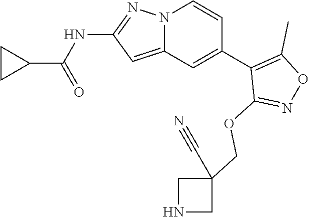 [SMILES: Cc1onc(OCC2(C#N)CNC2)c1-c1ccn2nc(NC(=O)C3CC3)cc2c1]

The title compound (38 mg, 4 steps, 2%) was prepared as described in Example 31, except tert-butyl 3-cyano-3-((tosyloxy)methyl)azetidine-1-carboxylate was used instead of (S)-2-((tert-butoxycarbonyl)amino)-2-phenylethyl 4-methylbenzenesulfonate in step A. MS (ESI): mass calcd. for C₂₀H₂₀N₆O₃, 392.4. m/z found, 393.1 [M+H]⁺. ¹H NMR (400 MHz, DMSO-d6) δ 11.07 (s, 1H), 8.58 (d, J=7.2 Hz, 1H), 7.66 (t, J=1.4 Hz, 1H), 6.90 (dd, J=7.2, 2.0 Hz, 1H), 6.84 (s, 1H), 4.72 (s, 2H), 3.79 (s, 2H), 3.59 (s, 2H), 2.55 (s, 3H), 2.01-1.84 (m, 1H), 0.92-0.70 (m, 4H).

### Example 78: N-[5-[5-methyl-3-[[(2R)-morpholin-2-yl]methoxy]isoxazol-4-yl]pyrazolo[1,5-a]pyridin-2-yl]cyclopropanecarboxamide

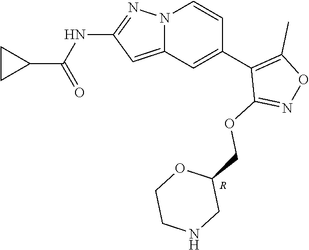

The title compound (76 mg, 3 steps, 11%) was prepared as described in Example 31, except (R)-tert-butyl-2-(bromomethyl)morpholine-4-carboxylate was used instead of (S)-2-((tert-butoxycarbonyl)amino)-2-phenylethyl 4-methylbenzenesulfonate in step A. MS (ESI): mass calcd. for C₂₀H₂₃N₅O₄, 397.4. m/z found, 398.2 [M+H]⁺. ¹H NMR (500 MHz, DMSO-d6) δ 11.05 (s, 1H), 8.58 (d, J=7.2 Hz, 1H), 7.64 (dd, J=2.0, 1.0 Hz, 1H), 6.90 (dd, J=7.2, 2.0 Hz, 1H), 6.85 (s, 1H), 4.25-4.19 (m, 2H), 3.82-3.66 (m, 2H), 3.50-3.40 (m, 1H), 2.84 (d, J=10.9 Hz, 1H), 2.70-2.59 (m, 2H), 2.52 (s, 3H), 1.96-1.88 (m, 1H), 0.87-0.76 (m, 4H). ¹H NMR (500 MHz, DMSO-d6) δ 11.05 (s, 1H), 8.61-8.52 (m, 1H), 7.64 (dd, J=2.0, 0.9 Hz, 1H), 6.90 (dd, J=7.2, 1.9 Hz, 1H), 6.85 (s, 1H), 4.25-4.19 (m, 2H), 3.78-3.69 (m, 2H), 3.49-3.40 (m, 1H), 3.29-3.26 (m, 1H), 2.84 (dd, J=12.1, 2.4 Hz, 1H), 2.68-2.58 (m, 2H), 2.52 (s, 3H), 1.95-1.89 (m, 1H), 0.86-0.77 (m, 4H).

### Example 79: N-[5-[5-methyl-3-[[(2R)-4-methylmorpholin-2-yl]methoxy]isoxazol-4-yl]pyrazolo[1,5-a]pyridin-2-yl]cyclopropanecarboxamide

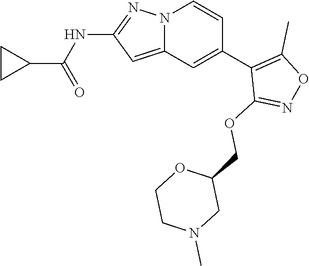 [SMILES: Cc1onc(OC[C@H]2CN(C)CCO2)c1-c1ccn2nc(NC(=O)C3CC3)cc2c1]

The title compound (52 mg, 83%) was prepared as described in Example 29 except N-[5-[5-methyl-3-[[(2R)-morpholin-2-yl]methoxy]isoxazol-4-yl]pyrazolo[1,5-a]pyridin-2-yl]cyclopropanecarboxamide was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. MS (ESI): mass calcd. for C₂₁H₂₅N₅O₄, 411.5. m/z found, 412.2 [M+H]⁺. ¹H NMR (500 MHz, DMSO-d6) δ 11.05 (s, 1H), 8.61-8.55 (m, 1H), 7.65 (dd, J=2.0, 0.9 Hz, 1H), 6.90 (dd, J=7.2, 2.0 Hz, 1H), 6.85 (s, 1H), 4.27 (d, J=4.9 Hz, 2H), 3.89-3.76 (m, 2H), 3.59-3.47 (m, 1H), 2.75 (d, J=11.2 Hz, 1H), 2.59 (d, J=11.5 Hz, 1H), 2.52 (s, 3H), 2.19 (s, 3H), 1.99 (t, J=11.7 Hz, 1H), 1.95-1.85 (m, 2H), 0.87-0.75 (m, 4H).

### Example 80: N-[5-[3-[(3S,4R)-4-fluoropyrrolidin-3-yl]oxy-5-methyl-isoxazol-4-yl]pyrazolo[1,5-a]pyridin-2-yl]cyclopropanecarboxamide

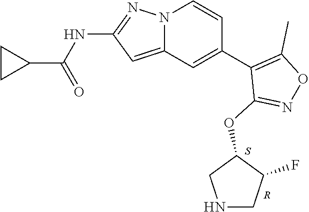

The title compound (16 mg, 3 steps, 5%) was prepared as described in Example 28, except tert-butyl (3R,4R)-3-fluoro-4-(tosyloxy)pyrrolidine-1-carboxylate was used instead of tert-butyl (S)-2-((tosyloxy)methyl)morpholine-4-carboxylate in step B. MS (ESI): mass calcd. for C₁₉H₂₀FN₅O₃, 385.4. m/z found, 386.1 [M+H]⁺. ¹H NMR (500 MHz, DMSO-d6) δ 11.04 (s, 1H), 8.57 (dt, J=7.2, 1.0 Hz, 1H), 7.64 (dd, J=2.0, 0.9 Hz, 1H), 6.90 (dd, J=7.2, 2.0 Hz, 1H), 6.85 (s, 1H), 5.36-5.27 (m, 1H), 5.24-5.14 (m, 1H), 5.05-4.89 (m, 1H), 3.26-3.15 (m, 1H), 2.96 (dd, J=26.5, 13.3 Hz, 1H), 2.90-2.82 (m, 1H), 2.53 (s, 3H), 1.97-1.87 (m, 1H), 0.89-0.73 (m, 4H).

### Example 81: N-[5-[5-methyl-3-[[(1S,4S)-5-oxa-2-azabicyclo[2.2.1]heptan-4-yl]methoxy]isoxazol-4-yl]pyrazolo[1,5-a]pyridin-2-yl]cyclopropanecarboxamide

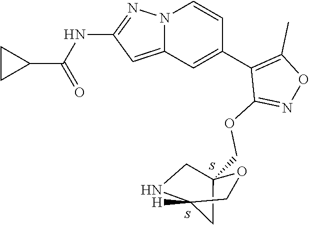

The title compound (62 mg, 3 steps, 16%) was prepared as described in Example 31, except tert-butyl (1S,4S)-1-(((methylsulfonyl)oxy)methyl)-2-oxa-5-azabicyclo[2.2.1]heptane-5-carboxylate was used instead of (S)-2-((tert-butoxycarbonyl)amino)-2-phenylethyl 4-methylbenzenesulfonate in step A. MS (ESI): mass calcd. for C₂₁H₂₃N₅O₄, 409.4. m/z found, 410.2 [M+H]⁺. ¹H NMR (500 MHz, DMSO-d6) δ 11.06 (s, 1H), 8.59 (d, J=7.2 Hz, 1H), 7.64 (dd, J=2.0, 0.9 Hz, 1H), 6.90 (dd, J=7.2, 2.0 Hz, 1H), 6.84 (s, 1H), 4.64-4.50 (m, 2H), 3.78 (dd, J=6.9, 1.8 Hz, 1H), 3.68 (d, J=6.9 Hz, 1H), 3.57 (s, 1H), 2.94 (d, J=9.7 Hz, 1H), 2.88 (d, J=9.7 Hz, 1H), 2.54 (s, 3H), 1.98-1.87 (m, 1H), 1.69 (d, J=2.1 Hz, 2H), 0.89-0.72 (m, 4H).

### Example 82: N-[5-[3-[(3S,4S)-4-fluoropyrrolidin-3-yl]oxy-5-methyl-isoxazol-4-yl]pyrazolo[1,5-a]pyridin-2-yl]cyclopropanecarboxamide

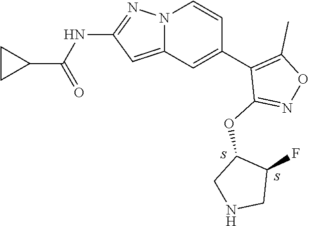

The title compound (29 mg, 3 steps, 32%) was prepared as described in Example 31, except tert-butyl (3 S,4R)-3-fluoro-4-((methylsulfonyl)oxy)pyrrolidine-1-carboxylate was used instead of (S)-2-((tert-butoxycarbonyl)amino)-2-phenylethyl 4-methylbenzenesulfonate in step A. MS (ESI): mass calcd. for C₁₉H₂₀FN₅O₃, 385.4. m/z found, 386.1 [M+H]⁺. ¹H NMR (400 MHz, DMSO-d6) δ 11.03 (s, 1H), 8.54 (d, J=6.9 Hz, 1H), 7.64 (s, 1H), 6.96-6.77 (m, 2H), 5.39-5.13 (m, 1H), 5.08 (d, J=17.0 Hz, 1H), 3.38-3.33 (m, 1H), 3.18-3.05 (m, 1H), 3.05-2.86 (m, 2H), 2.53 (s, 3H), 1.97-1.86 (m, 1H), 0.88-0.76 (m, 4H).

### Example 83: N-[5-[3-[(3-fluoroazetidin-3-yl)methoxy]-5-methyl-isoxazol-4-yl]pyrazolo[1,5-a]pyridin-2-yl]cyclopropanecarboxamide

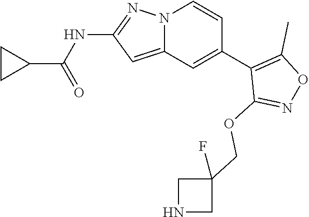 [SMILES: Cc1onc(OCC2(F)CNC2)c1-c1ccn2nc(NC(=O)C3CC3)cc2c1]

The title compound (69 mg, 3 steps, 23%) was prepared as described in Example 31, except tert-butyl 3-fluoro-3-((tosyloxy)methyl)azetidine-1-carboxylate was used instead of (S)-2-((tert-butoxycarbonyl)amino)-2-phenylethyl 4-methylbenzenesulfonate in step A. MS (ESI): mass calcd. for C₁₉H₂₀FN₅O₃, 385.4. m/z found, 386.2 [M+H]⁺. ¹H NMR (500 MHz, DMSO-d6) δ 11.05 (s, 1H), 8.58 (d, J=7.3 Hz, 1H), 7.62 (dd, J=2.0, 0.9 Hz, 1H), 6.87 (dd, J=7.2, 2.0 Hz, 1H), 6.84 (s, 1H), 4.70-4.58 (m, 2H), 3.70-3.60 (m, 2H), 3.57-3.48 (m, 2H), 2.54 (s, 3H), 1.99-1.85 (m, 1H), 0.92-0.71 (m, 4H).

### Example 84: N-[5-[3-[(3-fluoro-1-methyl-azetidin-3-yl)methoxy]-5-methyl-isoxazol-4-yl]pyrazolo[1,5-a]pyridin-2-yl]cyclopropanecarboxamide

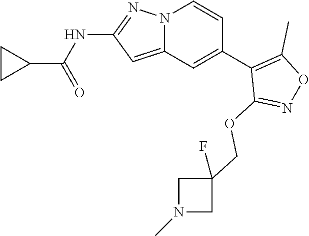 [SMILES: Cc1onc(OCC2(F)CN(C)C2)c1-c1ccn2nc(NC(=O)C3CC3)cc2c1]

The title compound (10 mg, 54%) was prepared as described in Example 29 except N-[5-[3-[(3-fluoroazetidin-3-yl)methoxy]-5-methyl-isoxazol-4-yl]pyrazolo[1,5-a]pyridin-2-yl]cyclopropanecarboxamide was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. MS (ESI): mass calcd. for C₂₀H₂₂FN₅O₃, 399.4. m/z found, 400.2 [M+H]⁺. ¹H NMR (500 MHz, DMSO-d6) δ 11.05 (s, 1H), 8.58 (dd, J=7.2, 1.0 Hz, 1H), 7.63 (dd, J=2.0, 1.0 Hz, 1H), 6.88 (dd, J=7.2, 2.0 Hz, 1H), 6.85 (s, 1H), 4.59 (d, J=23.9 Hz, 2H), 3.64-3.53 (m, 2H), 3.17 (dd, J=21.5, 8.6 Hz, 2H), 2.53 (s, 3H), 2.33 (s, 3H), 2.00-1.85 (m, 1H), 0.90-0.73 (m, 4H).

### Example 85: N-[5-[5-methyl-3-[(3S,5R)-5-methylpyrrolidin-3-yl]oxy-isoxazol-4-yl]pyrazolo[1,5-a]pyridin-2-yl]cyclopropanecarboxamide

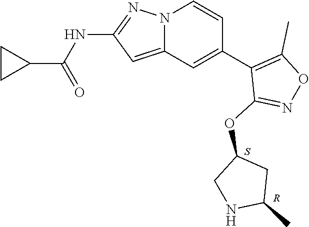

The title compound (61 mg, 3 steps, 28%) was prepared and described in Example 31, except tert-butyl (2R,4R)-2-methyl-4-(tosyloxy)pyrrolidine-1-carboxylate was used in Step A instead of (S)-2-((tert-butoxycarbonyl)amino)-2-phenylethyl 4-methylbenzenesulfonate. MS (ESI): mass calcd. for C₂₀H₂₃N₅O₃, 381.4. m/z found, 382.2 [M+H]⁺. ¹H NMR (500 MHz, DMSO-d6) δ 11.04 (s, 1H), 8.55 (d, J=7.2 Hz, 1H), 7.65 (d, J=2.0 Hz, 1H), 6.91 (dd, J=7.3, 2.0 Hz, 1H), 6.85 (s, 1H), 5.14-5.06 (m, 1H), 3.12 (d, J=13.0 Hz, 1H), 3.07-2.99 (m, 1H), 2.94 (dd, J=12.7, 5.4 Hz, 1H), 2.51 (s, 3H), 2.42-2.29 (m, 1H), 1.96-1.87 (m, 1H), 1.45-1.37 (m, 1H), 1.13 (d, J=6.3 Hz, 3H), 0.87-0.77 (m, 4H).

### Example 86: N-[5-[3-[[(2R)-6,6-dimethylmorpholin-2-yl]methoxy]-5-methyl-isoxazol-4-yl]pyrazolo[1,5-a]pyridin-2-yl]cyclopropanecarboxamide

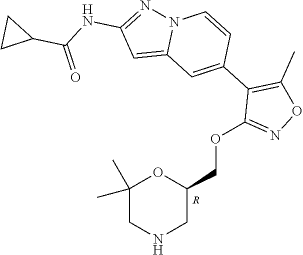

The title compound (115 mg, 3 steps, 27%) was prepared and described in Example 31, except tert-butyl (R)-2,2-dimethyl-6-((tosyloxy)methyl)morpholine-4-carboxylate was used in Step A instead of (S)-2-((tert-butoxycarbonyl)amino)-2-phenylethyl 4-methylbenzenesulfonate. MS (ESI): mass calcd. for C₂₂H₂₇N₅O₄, 425.5. m/z found, 426.2 [M+H]⁺. ¹H NMR (500 MHz, DMSO-d6) δ 11.06 (s, 1H), 8.58 (dd, J=7.2, 1.0 Hz, 1H), 7.68 (dd, J=2.0, 1.0 Hz, 1H), 6.93 (dd, J=7.2, 2.0 Hz, 1H), 6.84 (s, 1H), 4.18-4.10 (m, 2H), 4.04-3.92 (m, 1H), 2.89-2.79 (m, 1H), 2.57 (d, J=12.2 Hz, 1H), 2.53 (s, 3H), 2.44-2.31 (m, 2H), 1.96-1.88 (m, 1H), 1.25 (s, 3H), 1.07 (s, 3H), 0.88-0.76 (m, 4H).

### Example 87: N-[5-[5-methyl-3-[[(2R)-1-methylazetidin-2-yl]methoxy]isoxazol-4-yl]pyrazolo[1,5-a]pyridin-2-yl]cyclopropanecarboxamide

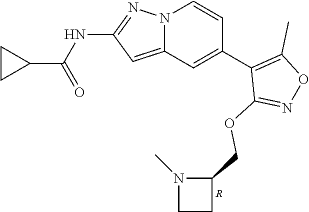

The title compound (51 mg, 18%) was prepared and described in Example 29, except (R)—N-(5-(3-(azetidin-2-ylmethoxy)-5-methylisoxazol-4-yl)pyrazolo[1,5-a]pyridin-2-yl)cyclopropanecarboxamide was used in instead of N-[5-[3-[(3-methoxyazetidin-3-yl)methoxy]-5-methyl-isoxazol-4-yl]pyrazolo[1,5-a]pyridin-2-yl]cyclopropanecarboxamide. (R)—N-(5-(3-(azetidin-2-ylmethoxy)-5-methylisoxazol-4-yl)pyrazolo[1,5-a]pyridin-2-yl)cyclopropanecarboxamide was prepared as described in Example 31 except tert-butyl (R)-2-((tosyloxy)methyl)azetidine-1-carboxylate was used instead of (S)-2-((tert-butoxycarbonyl)amino)-2-phenylethyl 4-methylbenzenesulfonate in Step A. MS (ESI): mass calcd. for C₂₀H₂₃N₅O₃, 381.4. m/z found, 382.2 [M+H]⁺. ¹H NMR (500 MHz, DMSO-d6) δ 11.05 (s, 1H), 8.60-8.53 (m, 1H), 7.65 (dd, J=2.0, 0.9 Hz, 1H), 6.91 (dd, J=7.2, 2.0 Hz, 1H), 6.83 (s, 1H), 4.27 (dd, J=10.6, 3.9 Hz, 1H), 4.20 (dd, J=10.6, 6.6 Hz, 1H), 3.39-3.33 (m, 1H), 3.30-3.24 (m, 1H), 2.79-2.67 (m, 1H), 2.52 (s, 3H), 2.23 (s, 3H), 2.03-1.86 (m, 3H), 0.88-0.76 (m, 4H).

### Example 88: N-[5-[3-[(3R,4S)-4-methoxypyrrolidin-3-yl]oxy-5-methyl-isoxazol-4-yl]pyrazolo[1,5-a]pyridin-2-yl]cyclopropanecarboxamide

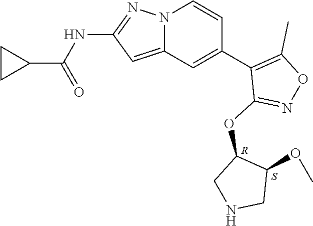

The title compound (218 mg, 3 steps, 20%) was prepared and described in Example 31, except tert-butyl (3 S,4S)-3-methoxy-4-(tosyloxy)pyrrolidine-1-carboxylate was used in Step A instead of (S)-2-((tert-butoxycarbonyl)amino)-2-phenylethyl 4-methylbenzenesulfonate. MS (ESI): mass calcd. for C₂₀H₂₃N₅O₄, 397.4. m/z found, 398.2 [M+H]⁺. ¹H NMR (500 MHz, DMSO-d6) δ 11.05 (s, 1H), 8.57 (d, J=7.2 Hz, 1H), 7.68 (d, J=2.0 Hz, 1H), 6.96 (dd, J=7.2, 2.0 Hz, 1H), 6.83 (s, 1H), 5.10-5.00 (m, 1H), 3.94-3.84 (m, 1H), 3.27 (s, 3H), 3.24 (dd, J=12.4, 5.6 Hz, 1H), 3.08 (dd, J=11.1, 6.4 Hz, 1H), 2.89 (dd, J=12.3, 4.1 Hz, 1H), 2.73 (dd, J=11.1, 6.7 Hz, 1H), 2.53 (s, 3H), 1.97-1.88 (m, 1H), 0.91-0.74 (m, 4H).

### Example 89: N-[5-[3-[[(2S)-3,3-dimethylazetidin-2-yl]methoxy]-5-methyl-isoxazol-4-yl]pyrazolo[1,5-a]pyridin-2-yl]cyclopropanecarboxamide

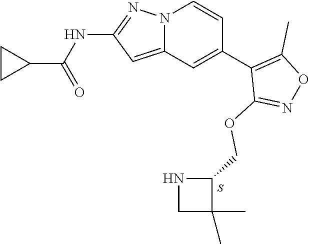

The title compound (79 mg, 3 steps, 26%) was prepared and described in Example 31, except modified step A as described below was followed. MS (ESI): mass calcd. for C₂₁H₂₅N₅O₃, 395.5. m/z found, 396.2 [M+H]⁺. ¹H NMR (400 MHz, DMSO-d6) δ 11.04 (s, 1H), 8.56 (dt, J=7.2, 0.9 Hz, 1H), 7.62 (dd, J=2.0, 1.0 Hz, 1H), 6.88 (dd, J=7.2, 2.0 Hz, 1H), 6.83 (s, 1H), 4.28 (dd, J=10.3, 6.9 Hz, 1H), 4.20 (dd, J=10.3, 6.4 Hz, 1H), 3.74 (t, J=6.6 Hz, 1H), 3.23 (d, J=6.6 Hz, 1H), 2.90 (d, J=6.6 Hz, 1H), 2.52 (s, 3H), 1.99-1.85 (m, 1H), 1.15 (s, 6H), 0.91-0.75 (m, 4H). Modification to Step A: To a solution of 4-bromo-5-methylisoxazol-3-ol (250 mg, 1.40 mmol), tert-butyl (S)-2-(hydroxymethyl)-3,3-dimethylazetidine-1-carboxylate (302 mg, 1.40 mmol), di-tert-butyl azodicarboxylate (420 mg, 1.83 mmol) in THE (5.2 mL, 0.2 M) was added triphenylphosphine (480 mg, 1.83 mmol) in one portion at 23° C. After 1 hour, the mixture was concentrated in vacuo and purified by silica gel chromatography (0-50% EtOAc in hexanes) to provide tert-butyl (S)-2-(((4-bromo-5-methylisoxazol-3-yl)oxy)methyl)-3,3-dimethylazetidine-1-carboxylate as clear colorless oil which was used as is in the following reaction.

### Example 90: N-(2,6-dimethylpyrimidin-4-yl)-5-[5-methyl-3-[(3S,5R)-5-methylpyrrolidin-3-yl]oxy-isoxazol-4-yl]pyrazolo[1,5-a]pyridin-2-amine

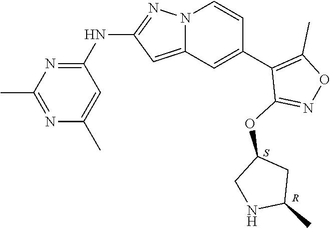

Step A. Tert-butyl (2R,4S)-4-((4-(2-aminopyrazolo[1,5-a]pyridin-5-yl)-5-methylisoxazol-3-yl)oxy)-2-methylpyrrolidine-1-carboxylate. The title compound (43 mg, 4 steps, 25%) was prepared and described in Example 31 except tert-butyl (2R,4R)-2-methyl-4-(tosyloxy)pyrrolidine-1-carboxylatecarboxylate was used instead of (S)-2-((tert-butoxycarbonyl)amino)-2-phenylethyl 4-methylbenzenesulfonate in step A; 5-(4,4,5,5-tetramethyl-1,3,2-dioxaborolan-2-yl)pyrazolo[1,5-a]pyridin-2-amine was used instead of N-(5-(4,4,5,5-tetramethyl-1,3,2-dioxaborolan-2-yl)pyrazolo[1,5-a]pyridin-2-yl)cyclopropanecarboxamide in step B to give tert-butyl (2R,4S)-4-((4-(2-aminopyrazolo[1,5-a]pyridin-5-yl)-5-methylisoxazol-3-yl)oxy)-2-methylpyrrolidine-1-carboxylate which was used as in the following reaction.

Step B: Tert-butyl (2R,4S)-4-((4-(2-((2,6-dimethylpyrimidin-4-yl)amino)pyrazolo[1,5-a]pyridin-5-yl)-5-methylisoxazol-3-yl)oxy)-2-methylpyrrolidine-1-carboxylate. To a vial charged with tert-butyl (2R,4S)-4-((4-(2-aminopyrazolo[1,5-a]pyridin-5-yl)-5-methylisoxazol-3-yl)oxy)-2-methylpyrrolidine-1-carboxylate (100 mg, 0.242 mmol), 4-bromo-2,6-dimethylpyrimidine (67 mg, 0.363 mmol), Brettphos-Pd-G3 (22 mg, 0.024 mmol), cesium carbonate (304 mg, 0.93 mmol) and 1,4-dioxane (1.0 mL) was evacuated and backfilled with Argon. This was repeated two more times and the resulting solution was then stirred at 100 C. After 2 hours, the reaction mixture was cooled to 23° C. and then filtered through celite and the solvent removed in vacuo. The resulting oil was purified by silica gel chromatography (0-100% EtOAc in hexanes) to afford the title compound (85 mg, 0.164 mmol, 67% yield). MS (ESI): mass calcd. for C₂₇H₃₃N₇O₄, 519.6. m/z found, 520.3 [M+H]⁺.

Step C: N-(2,6-dimethylpyrimidin-4-yl)-5-[5-methyl-3-[(3S,5R)-5-methylpyrrolidin-3-yl]oxy-isoxazol-4-yl]pyrazolo[1,5-a]pyridin-2-amine. To a vial containing tert-butyl (2R,4S)-4-((4-(2-((2,6-dimethylpyrimidin-4-yl)amino)pyrazolo[1,5-a]pyridin-5-yl)-5-methylisoxazol-3-yl)oxy)-2-methylpyrrolidine-1-carboxylate (85 mg, 0.164 mmol) was added trifluoroacetic acid (0.45 mL) and the resulting mixture allowed to stand at 23° C. for 10 minutes. The solvent was removed in vacuo at the resulting oil was purified by reverse phase using 10-80% MeCN in water containing 20 mM NH₄OH. The fractions containing the desired product were pooled together and lyophilized to provide titled compound (43 mg, 0.103 mmol, 62%) as a white solid. MS (ESI): mass calcd. for C₂₂H₂₅N₇O₂, 419.5. m/z found, 420.2 [M+H]⁺. ¹H NMR (400 MHz, DMSO-d6) δ 10.17 (s, 1H), 8.57 (dd, J=7.2, 1.0 Hz, 1H), 7.65 (dd, J=2.0, 0.9 Hz, 1H), 7.00 (s, 1H), 6.89 (dd, J=7.2, 2.0 Hz, 1H), 6.83 (s, 1H), 5.16-5.06 (m, 1H), 3.16-3.10 (m, 1H), 3.03 (q, J=6.9 Hz, 1H), 2.96 (dd, J=12.7, 5.5 Hz, 1H), 2.52 (s, 3H), 2.46 (s, 3H), 2.42-2.34 (m, 1H), 2.30 (s, 3H), 1.46-1.37 (m, 1H), 1.14 (d, J=6.3 Hz, 3H).

### Example 91: N-(2,6-dimethylpyrimidin-4-yl)-5-[5-methyl-3-[[(2R)-1-methylazetidin-2-yl]methoxy]isoxazol-4-yl]pyrazolo[1,5-a]pyridin-2-amine

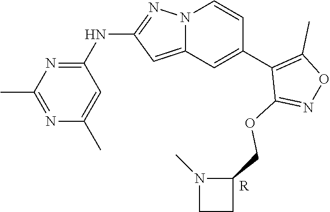

The title compound (20 mg, 5 steps, 11%) was prepared as described in Example 90 except tert-butyl (R)-2-((tosyloxy)methyl)azetidine-1-carboxylate was used instead of tert-butyl (2R,4R)-2-methyl-4-(tosyloxy)pyrrolidine-1-carboxylatecarboxylate in step A; and the final step follows Example 29. MS (ESI): mass calcd. for C₂₂H₂₅N₇O₂, 419.5. m/z found, 420.2 [M+H]⁺. ¹H NMR (500 MHz, DMSO-d6) δ 10.17 (s, 1H), 8.60 (dt, J=7.3, 1.0 Hz, 1H), 7.65 (dd, J=2.0, 0.9 Hz, 1H), 7.00 (s, 1H), 6.88 (dd, J=7.2, 2.0 Hz, 1H), 6.82 (s, 1H), 4.28 (dd, J=10.7, 3.9 Hz, 1H), 4.21 (dd, J=10.7, 6.6 Hz, 1H), 3.41-3.34 (m, 1H), 3.29-3.24 (m, 1H), 2.80-2.71 (m, 1H), 2.53 (s, 3H), 2.45 (s, 3H), 2.30 (s, 3H), 2.24 (s, 3H), 2.03-1.88 (m, 2H).

### Example 92: N-(2,6-dimethylpyrimidin-4-yl)-5-[3-[(3R,4S)-4-fluoropyrrolidin-3-yl]oxy-5-methyl-isoxazol-4-yl]pyrazolo[1,5-a]pyridin-2-amine

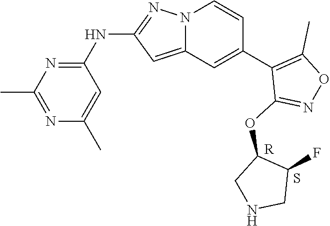

The title compound (45 mg, 4 steps, 21%) was prepared as described in Example 89 except tert-butyl (3S,4S)-3-fluoro-4-hydroxypyrrolidine-1-carboxylate was used instead of tert-butyl (S)-2-(hydroxymethyl)-3,3-dimethylazetidine-1-carboxylate in Step A; however, Steps B-D were followed as described in Example 90. MS (ESI): mass calcd. for C₂₁H₂₂FN₇O₂, 423.4. m/z found, 424.3 [M+H]⁺. ¹H NMR (400 MHz, DMSO-d6) δ 10.17 (s, 1H), 8.59 (d, J=7.2 Hz, 1H), 7.64 (d, J=1.8 Hz, 1H), 6.99 (s, 1H), 6.88 (dd, J=7.2, 2.0 Hz, 1H), 6.84 (s, 1H), 5.37-5.16 (m, 1H), 5.06-4.92 (m, 1H), 3.28-3.16 (m, 3H), 2.97 (ddd, J=26.6, 13.4, 2.3 Hz, 1H), 2.87 (dd, J=11.5, 7.5 Hz, 1H), 2.54 (s, 3H), 2.46 (s, 3H), 2.30 (s, 3H).

### Example 93: N-(5,6-dimethylpyridazin-3-yl)-5-[3-[(3R,4S)-4-fluoropyrrolidin-3-yl]oxy-5-methyl-isoxazol-4-yl]pyrazolo[1,5-a]pyridin-2-amine

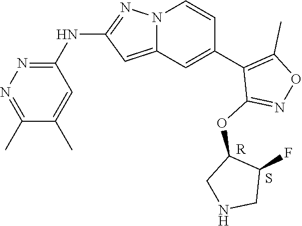

The title compound (33 mg, 4 steps, 16%) was prepared as described in Example 92 except 6-chloro-3,4-dimethylpyridine was used instead of 4-bromo-2,6-dimethylpyrimidine in Step C. MS (ESI): mass calcd. for C₂₁H₂₂FN₇O₂, 423.4. m/z found, 424.2 [M+H]⁺. ¹H NMR (400 MHz, DMSO-d6) δ 9.87 (s, 1H), 8.57 (dd, J=7.2, 1.0 Hz, 1H), 7.62 (dd, J=2.0, 0.9 Hz, 1H), 7.39 (d, J=1.3 Hz, 1H), 6.87-6.81 (m, 2H), 5.36-5.17 (m, 1H), 5.06-4.93 (m, 1H), 3.30-3.16 (m, 2H), 3.04-2.91 (m, 1H), 2.91-2.84 (m, 1H), 2.54 (s, 3H), 2.46 (s, 3H), 2.24 (s, 3H).

### Example 94: N-(2,6-dimethylpyrimidin-4-yl)-5-[3-[(3R,4S)-4-methoxypyrrolidin-3-yl]oxy-5-methyl-isoxazol-4-yl]pyrazolo[1,5-a]pyridin-2-amine

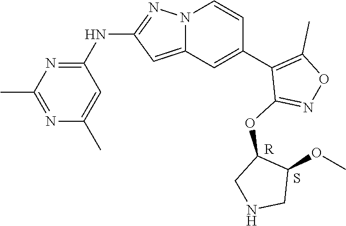

The title compound (40 mg, 4 steps, 16%) was prepared as described in Example 92 except tert-butyl (3S,4S)-3-hydroxy-4-methoxypyrrolidine-1-carboxylate was used instead of tert-butyl (3S,4S)-3-fluoro-4-hydroxypyrrolidine-1-carboxylate in Step A. MS (ESI): mass calcd. for C₂₂H₂₅N₇O₃, 435.5. m/z found, 436.2 [M+H]⁺. ¹H NMR (400 MHz, DMSO-d6) δ 10.18 (s, 1H), 8.59 (d, J=7.1 Hz, 1H), 7.67 (s, 1H), 7.04-6.89 (m, 2H), 6.86-6.73 (m, 1H), 5.10-5.02 (m, 1H), 3.95-3.84 (m, 1H), 3.28 (s, 3H), 3.26-3.21 (m, 1H), 3.08 (dd, J=11.1, 6.2 Hz, 1H), 2.90 (dd, J=12.1, 4.3 Hz, 1H), 2.74 (dd, J=11.2, 6.6 Hz, 1H), 2.54 (s, 3H), 2.45 (s, 3H), 2.29 (s, 3H).

### Example 95: N-(5,6-dimethylpyridazin-3-yl)-5-[3-[(3R,4S)-4-methoxypyrrolidin-3-yl]oxy-5-methyl-isoxazol-4-yl]pyrazolo[1,5-a]pyridin-2-amine

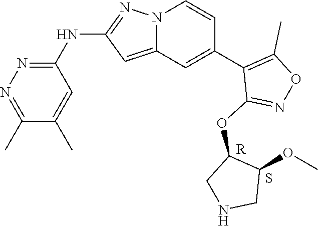

The title compound (33 mg, 4 steps, 13%) was prepared as described in Example 94 except 6-chloro-3,4-dimethylpyridine was used instead of 4-bromo-2,6-dimethylpyrimidine in Step C. MS (ESI): mass calcd. for C₂₂H₂₅N₇O₃, 435.5. m/z found, 436.3 [M+H]⁺. ¹HNMR (400 MHz, DMSO-d6) δ 9.88 (s, 1H), 8.56 (d, J=7.2 Hz, 1H), 7.66 (d, J=2.0 Hz, 1H), 7.38 (s, 1H), 6.90 (dd, J=7.2, 2.0 Hz, 1H), 6.83 (s, 1H), 5.11-5.04 (m, 1H), 3.96-3.88 (m, 1H), 3.28 (s, 3H), 3.25 (dd, J=12.3, 5.5 Hz, 1H), 3.09 (dd, J=11.1, 6.4 Hz, 1H), 2.90 (dd, J=12.4, 4.1 Hz, 1H), 2.75 (dd, J=11.1, 6.7 Hz, 1H), 2.54 (s, 3H), 2.46 (s, 3H), 2.24 (s, 3H).

### Example 96: 5-[2-(difluoromethyl)-4-[(3R,4S)-4-methoxypyrrolidin-3-yl]oxy-pyrazol-3-yl]-N-(2,6-dimethylpyrimidin-4-yl)pyrazolo[1,5-a]pyridin-2-amine

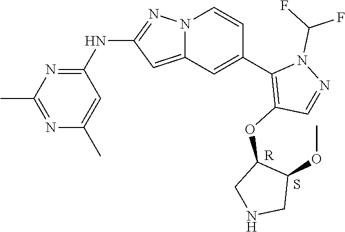

The title compound (77 mg, 4 steps, 13%) was prepared as described in Example 32, except tert-butyl (3S,4S)-3-methoxy-4-(tosyloxy)pyrrolidine-1-carboxylate was used instead of tert-butyl (3S,4S)-3-fluoro-4-(tosyloxy)pyrrolidine-1-carboxylate in Step A. MS (ESI): mass calcd. for C₂₂H₂₄F₂N₈O₂, 470.2. m/z found, 470.3 [M+H]⁺. ¹H NMR (500 MHz, DMSO-d6) δ 10.23 (s, 1H), 8.65 (dd, J=7.1, 1.0 Hz, 1H), 7.96 (s, 1H), 7.87-7.60 (m, 2H), 6.99 (s, 1H), 6.92 (s, 1H), 6.89 (dd, J=7.2, 2.0 Hz, 1H), 4.66 (dt, J=5.4, 4.2 Hz, 1H), 3.83 (td, J=6.5, 4.7 Hz, 1H), 3.22 (s, 3H), 3.13 (dd, J=12.0, 5.5 Hz, 1H), 3.02 (dd, J=11.0, 6.3 Hz, 1H), 2.82 (dd, J=12.1, 4.0 Hz, 1H), 2.70 (dd, J=11.0, 6.7 Hz, 1H), 2.46 (s, 3H), 2.30 (s, 3H).

### Example 97: 5-[2-(difluoromethyl)-4-[[(2R)-1-methylazetidin-2-yl]methoxy]pyrazol-3-yl]-N-(6-methoxy-5-methyl-pyridazin-3-yl)pyrazolo[1,5-a]pyridin-2-amine

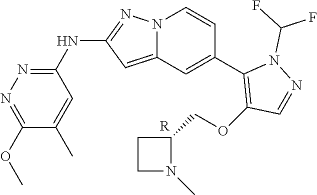

The title compound (46 mg, 2 steps, 22%) was prepared as described in Example 33, except 6-chloro-3-methoxy-4-methylpyridazine was used instead of 6-chloro-2,4-dimethylpyridazin-3(2H)-one in Step A. MS (ESI): mass calcd. for C₂₂H₂₄F₂N₈O₂, 470.2. m/z found, 471.2 [M+H]⁺.

¹H NMR (600 MHz, DMSO-d6) δ 9.83 (s, 1H), 8.61 (dt, J=7.2, 0.9 Hz, 1H), 7.94 (s, 1H), 7.84-7.59 (m, 2H), 7.35 (d, J=1.2 Hz, 1H), 6.99 (d, J=0.8 Hz, 1H), 6.79 (dd, J=7.1, 2.0 Hz, 1H), 4.06-3.98 (m, 2H), 3.97 (s, 3H), 3.25-3.18 (m, 2H), 2.74-2.66 (m, 1H), 2.16 (d, J=1.5 Hz, 6H), 1.96-1.88 (m, 1H), 1.84 (p, J=9.0 Hz, 1H).

### Example 98: 5-[2-(difluoromethyl)-4-[[(2R)-1-methylazetidin-2-yl]methoxy]pyrazol-3-yl]-N-(6-methylpyridazin-3-yl)pyrazolo[1,5-a]pyridin-2-amine

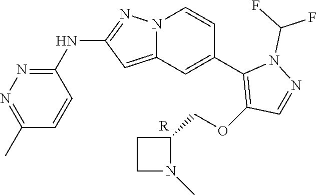

The title compound (20 mg, 2 steps, 10%) was prepared as described in Example 33, except 3-bromo-6-methylpyridazine was used instead of 6-chloro-2,4-dimethylpyridazin-3(2H)-one in Step A. MS (ESI): mass calcd. for C₂₁H₂₂F₂N₈O, 440.2. m/z found, 441.2 [M+H]⁺. ¹H NMR (600 MHz, DMSO-d6) δ 10.06 (s, 1H), 8.64-8.60 (m, 1H), 7.95 (s, 1H), 7.84-7.61 (m, 2H), 7.56 (d, J=9.1 Hz, 1H), 7.41 (d, J=9.1 Hz, 1H), 6.92 (d, J=0.9 Hz, 1H), 6.80 (dd, J=7.1, 2.0 Hz, 1H), 4.07-3.96 (m, 2H), 3.30 (s, 3H), 3.24-3.18 (m, 2H), 2.72-2.66 (m, 1H), 2.16 (s, 3H), 1.95-1.89 (m, 1H), 1.84 (p, J=9.0 Hz, 1H).

### Example 99: (2R,3S)-2-[[1-(difluoromethyl)-5-[2-[(2,6-dimethylpyrimidin-4-yl)amino]pyrazolo[1,5-a]pyridin-5-yl]pyrazol-4-yl]oxymethyl]tetrahydrofuran-3-ol

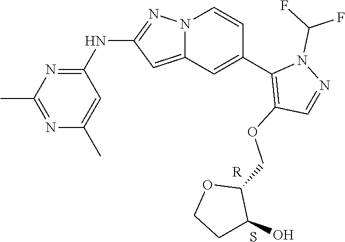

The title compound (52 mg, 3 steps, 21%) was prepared as described in Example 32 Steps A-C, except ((2R,3S)-3-hydroxytetrahydrofuran-2-yl)methyl 4-methylbenzenesulfonate was used instead of tert-butyl (3S,4S)-3-fluoro-4-(tosyloxy)pyrrolidine-1-carboxylate in Step A. MS (ESI): mass calcd. for C₂₂H₂₃F₂N₇O₃, 471.2. m/z found, 472.2 [M+H]⁺. ¹H NMR (500 MHz, DMSO-d6) δ 10.23 (s, 1H), 8.66 (dt, J=7.2, 0.9 Hz, 1H), 7.94 (s, 1H), 7.87-7.60 (m, 2H), 6.99 (s, 1H), 6.93 (s, 1H), 6.83 (dd, J=7.2, 2.0 Hz, 1H), 5.04 (d, J=3.6 Hz, 1H), 4.12-4.08 (m, 1H), 4.08-3.98 (m, 2H), 3.84-3.72 (m, 3H), 2.46 (s, 3H), 2.30 (s, 3H), 2.00-1.87 (m, 1H), 1.75-1.65 (m, 1H).

### Example 100: (RS)-5-[2-(difluoromethyl)-4-[(3-methoxypyrrolidin-3-yl)methoxy]pyrazol-3-yl]-N-(2,6-dimethylpyrimidin-4-yl)pyrazolo[1,5-a]pyridin-2-amine

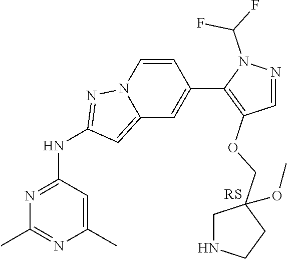

The title compound (80 mg, 4 steps, 30%) was prepared as described in Example 32, except tert-butyl 3-methoxy-3-((tosyloxy)methyl)pyrrolidine-1-carboxylate was used instead of tert-butyl (3S,4S)-3-fluoro-4-(tosyloxy)pyrrolidine-1-carboxylate in Step A. MS (ESI): mass calcd. for C₂₃H₂₆F₂N₈O₂, 484.2. m/z found, 485.2 [M+H]⁺. ¹H NMR (500 MHz, DMSO-d6) δ 10.24 (s, 1H), 8.67 (dd, J=7.2, 1.0 Hz, 1H), 7.99 (s, 1H), 7.90-7.58 (m, 2H), 7.00 (s, 1H), 6.92 (s, 1H), 6.85 (dd, J=7.2, 2.0 Hz, 1H), 4.17 (d, J=10.5 Hz, 1H), 4.09 (d, J=10.5 Hz, 1H), 3.13 (s, 3H), 2.86-2.77 (m, 1H), 2.77-2.70 (m, 3H), 2.46 (s, 3H), 2.30 (s, 3H), 1.80-1.71 (m, 1H), 1.64-1.54 (m, 1H).

### Example 101: (RS)-5-[2-(difluoromethyl)-4-[(3-fluoropyrrolidin-3-yl)methoxy]pyrazol-3-yl]-N-(2,6-dimethylpyrimidin-4-yl)pyrazolo[1,5-a]pyridin-2-amine

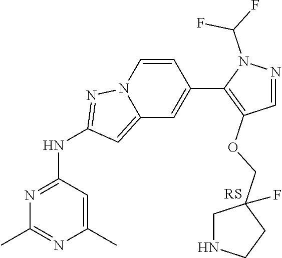 [SMILES: Cc1cc(Nc2cc3cc(-c4c(OCC5(F)CCNC5)cnn4C(F)F)ccn3n2)nc(C)n1]

The title compound (195 mg, 4 steps, 38%) was prepared as described in Example 32, except tert-butyl 3-fluoro-3-((tosyloxy)methyl)pyrrolidine-1-carboxylate was used instead of tert-butyl (3S,4S)-3-fluoro-4-(tosyloxy)pyrrolidine-1-carboxylate in Step A. MS (ESI): mass calcd. for C₂₂H₂₃F₃N₈O, 472.2. m/z found, 473.2 [M+H]⁺. ¹H NMR (500 MHz, DMSO-d6) δ 10.24 (s, 1H), 8.69-8.64 (m, 1H), 7.98 (s, 1H), 7.87-7.61 (m, 2H), 6.99 (s, 1H), 6.94 (s, 1H), 6.84 (dd, J=7.1, 2.0 Hz, 1H), 4.36-4.22 (m, 2H), 2.94-2.83 (m, 3H), 2.82-2.76 (m, 1H), 2.46 (s, 3H), 2.30 (s, 3H), 1.95-1.76 (m, 2H).

### Example 102: N-[2-(difluoromethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine

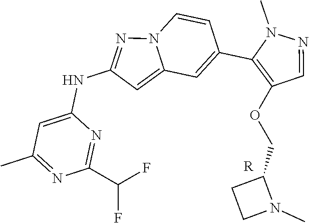

The title compound (232 mg, 3 steps, 53%) was prepared as described in Example 2, except 4-bromo-2-(difluoromethyl)-6-methylpyrimidine was used instead of 4-chloro-2,6-dimethylpyrimidine in Step A. MS (ESI): mass calcd. for C₂₂H₂₄F₂N₈O, 454.2. m/z found, 455.2 [M+H]⁺. ¹H NMR (500 MHz, Chloroform-d) δ 8.95 (s, 1H), 8.63 (d, J=7.2 Hz, 1H), 7.49 (dd, J=1.9, 0.9 Hz, 1H), 7.36 (s, 1H), 7.26 (s, 1H), 6.90 (dd, J=7.2, 1.9 Hz, 1H), 6.63 (d, J=10.3 Hz, 1H), 6.53 (s, 1H), 4.00 (d, J=5.5 Hz, 2H), 3.92 (s, 3H), 3.50-3.40 (m, 1H), 3.38-3.30 (m, 1H), 2.87-2.78 (m, 1H), 2.52 (s, 3H), 2.34 (s, 3H), 2.07-1.95 (m, 2H).

### Example 103: N-[2-(difluoromethyl)-6-methyl-pyrimidin-4-yl]-5-[4-[[(2R)-1-ethylazetidin-2-yl]methoxy]-2-methyl-pyrazol-3-yl]pyrazolo[1,5-a]pyridin-2-amine

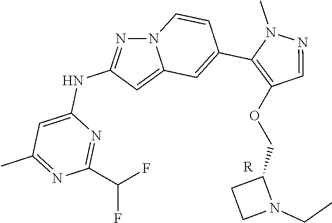

The title compound (48 mg, 64%) was prepared as described in Example 102 except in Step C, acetaldehyde was used instead of paraformaldehyde. MS (ESI): mass calcd. for C₂₃H₂₆F₂N₈O, 468.2. m/z found, 469.2 [M+H]⁺. ¹H NMR (600 MHz, Chloroform-d) δ 8.64 (s, 1H), 8.60 (d, J=7.2 Hz, 1H), 7.50 (dd, J=2.0, 0.9 Hz, 1H), 7.35 (s, 1H), 7.28 (s, 1H), 6.89 (dd, J=7.2, 1.9 Hz, 1H), 6.62 (s, 1H), 6.53 (s, 1H), 4.03 (dd, J=9.8, 6.4 Hz, 1H), 3.98 (dd, J=9.8, 4.7 Hz, 1H), 3.92 (s, 3H), 3.45-3.34 (m, 2H), 2.80-2.74 (m, 1H), 2.72-2.66 (m, 1H), 2.53 (s, 3H), 2.38-2.32 (m, 1H), 2.11-1.94 (m, 2H), 0.95 (t, J=7.2 Hz, 3H).

### Example 104: 5-[2-(difluoromethyl)-4-[[(2R)-1-ethylazetidin-2-yl]methoxy]pyrazol-3-yl]-N-(1-methylpyrazol-4-yl)pyrazolo[1,5-a]pyridin-2-amine

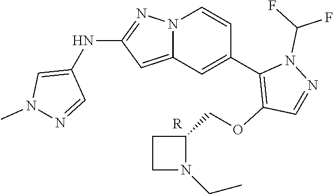

The title compound was prepared as described in Example 33, except 4-bromo-1-methyl-1H-pyrazole and acetaldehyde was used instead of 6-chloro-2,4-dimethylpyridazin-3(2H)-one and formaldehyde in Step A (35 mg, 2 steps, 24%). MS (ESI): mass calcd. for C₂₁H₂₄F₂N₈O, 442.2. m/z found, 443.2 [M+H]⁺. ¹H NMR (400 MHz, DMSO-d6) δ 9.10 (s, 1H), 8.51 (dt, J=7.1, 0.9 Hz, 1H), 7.92 (s, 1H), 7.70 (t, J=57.9 Hz, 1H), 7.52-7.44 (m, 2H), 6.68 (dd, J=7.1, 2.0 Hz, 1H), 6.47 (d, J=0.9 Hz, 1H), 6.05 (d, J=2.2 Hz, 1H), 4.05-3.95 (m, 2H), 3.73 (s, 3H), 3.22 (s, 2H), 2.69-2.60 (m, 1H), 2.58-2.52 (m, 1H), 2.21 (dq, J=11.4, 7.1 Hz, 1H), 1.97-1.86 (m, 1H), 1.86-1.75 (m, 1H), 0.79 (t, J=7.2 Hz, 3H).

### Example 105: N-[5-[2-methyl-4-[(3S)-1-methylpyrrolidin-3-yl]oxy-pyrazol-3-yl]pyrazolo[1,5-a]pyridin-2-yl]cyclopropanecarboxamide

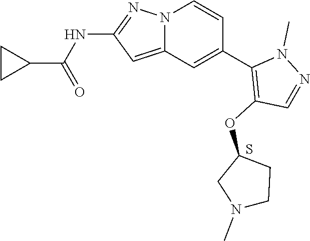

The title compound was prepared as described in Example 32, except (R)-tert-butyl 3-(tosyloxy)pyrrolidine-1-carboxylate was used instead of tert-butyl (3S,4S)-3-fluoro-4-(tosyloxy)pyrrolidine-1-carboxylate in Step A (40.1 mg, 3 steps, 22%). MS (ESI): mass calcd. for C₂₀H₂₄N₆O₂, 380.2. m/z found, 381.3 [M+H]⁺. ¹H NMR (500 MHz, DMSO-d6) δ 11.07 (s, 1H), 8.60 (d, J=7.2 Hz, 1H), 7.71-7.65 (m, 1H), 7.39 (s, 1H), 6.95 (dd, J=7.2, 2.0 Hz, 1H), 6.89 (s, 1H), 4.69-4.63 (m, 1H), 3.85 (s, 3H), 2.67 (dd, J=10.5, 5.9 Hz, 1H), 2.64-2.57 (m, 2H), 2.34-2.27 (m, 1H), 2.21 (s, 3H), 2.20-2.13 (m, 1H), 1.97-1.90 (m, 1H), 1.80-1.73 (m, 1H), 0.88-0.77 (m, 4H).

### Example 106: N-(2-hydroxy-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide

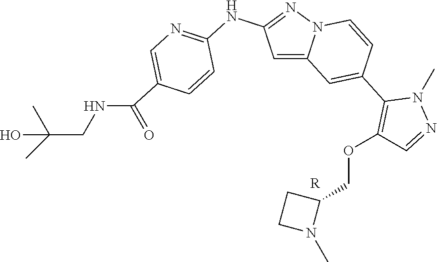

The title compound (33.5 mg, 3 steps, 19%) was prepared as described in Example 36, except 1-amino-2-methylpropan-2-ol was used instead of 3-amino-2,2-dimethylpropanenitrile in Step C. MS (ESI): mass calcd. for C₂₆H₃₂N₈O₃, 504.3. m/z found, 505.5 [M+H]⁺. ¹H NMR (400 MHz, DMSO-d6) δ 10.22 (s, 1H), 8.75 (d, J=2.4 Hz, 1H), 8.61 (d, J=7.2 Hz, 1H), 8.20 (t, J=5.6 Hz, 1H), 8.08 (dd, J=2.4, 8.8 Hz, 1H), 7.68 (d, J=1.2 Hz, 1H), 7.45 (s, 1H), 7.36 (d, J=8.8 Hz, 1H), 6.91 (dd, J=2.0, 6.8 Hz, 1H), 6.89 (s, 1H), 4.59 (br s, 1H), 3.98-3.89 (m, 2H), 3.87 (s, 3H), 3.30-3.28 (m, 1H), 3.28-3.19 (m, 3H), 2.76-2.65 (m, 1H), 2.19 (s, 3H), 1.99-1.77 (m, 2H), 1.11 (s, 6H); ¹H NMR (400 MHz, CD3OD): 8.74 (d, J=2.0 Hz, 1H), 8.47 (d, J=7.2 Hz, 1H), 8.08 (dd, J=2.4, 8.8 Hz, 1H), 7.62 (d, J=0.4 Hz, 1H), 7.44 (s, 1H), 7.41 (d, J=8.8 Hz, 1H), 6.92 (dd, J=2.0, 7.2 Hz, 1H), 6.84 (s, 1H), 4.09-3.94 (m, 2H), 3.90 (s, 3H), 3.52-3.43 (m, 1H), 3.41 (s, 2H), 3.39-3.34 (m, 1H), 2.96-2.85 (m, 1H), 2.32 (s, 3H), 2.14-1.91 (m, 2H), 1.24 (s, 6H)

### Example 107: N-(2-methoxyethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide

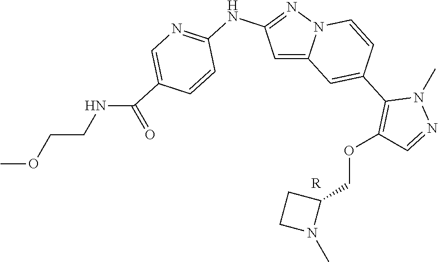

The title compound (26.5 mg, 5 steps, 4%) was prepared as described in Example 36, except 2-methoxyethanamine was used instead of 3-amino-2,2-dimethylpropanenitrile in Step C. MS (ESI): mass calcd. for C₂₅H₃₀N₈O₃, 490.2. m/z found, 491.3 [M+H]⁺. ¹H NMR (400 MHz, DMSO-d6) δ 10.23 (s, 1H), 8.72 (d, J=2.0 Hz, 1H), 8.61 (d, J=7.2 Hz, 1H), 8.49-8.40 (m, 1H), 8.06 (dd, J=2.8, 8.8 Hz, 1H), 7.68 (d, J=0.8 Hz, 1H), 7.45 (s, 1H), 7.35 (d, J=8.8 Hz, 1H), 6.91 (dd, J=2.0, 7.2 Hz, 1H), 6.89 (s, 1H), 3.98-3.89 (m, 2H), 3.87 (s, 3H), 3.50-3.40 (m, 5H), 3.28 (s, 3H), 3.26-3.19 (m, 1H), 2.76-2.64 (m, 1H), 2.19 (s, 3H), 1.99-1.76 (m, 2H); ¹H NMR (400 MHz, CD3OD) δ8.78-8.64 (m, 1H), 8.46 (d, J=7.2 Hz, 1H), 8.05 (dd, J=2.4, 8.8 Hz, 1H), 7.62 (d, J=0.8 Hz, 1H), 7.44 (s, 1H), 7.39 (d, J=8.8 Hz, 1H), 6.92 (dd, J=2.0, 7.2 Hz, 1H), 6.84 (s, 1H), 4.09-3.95 (m, 2H), 3.90 (s, 3H), 3.61-3.52 (m, 4H), 3.51-3.42 (m, 1H), 3.39 (s, 3H), 3.37-3.33 (m, 1H), 2.97-2.85 (m, 1H), 2.32 (s, 3H), 2.12-1.92 (m, 2H)

### Example 108: N,N,2-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide

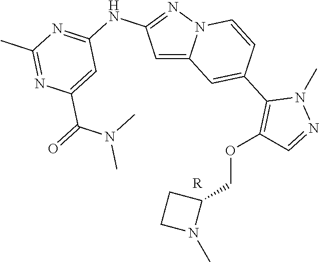

The title compound (64.9 mg, 5 steps, 14%) was prepared as described in Example 36, except ethyl 6-chloro-2-methylpyrimidine-4-carboxylate was used instead of methyl 6-bromonicotinate in Step A and dimethylamine hydrochloride was used instead of 3-amino-2,2-dimethylpropanenitrile in Step C. MS (ESI): mass calcd. for C₂₄H₂₉N₉O₂, 475.2. m/z found, 476.2 [M+H]⁺. ¹H NMR (400 MHz, DMSO-d6) δ 10.59 (s, 1H), 8.66 (d, J=7.2 Hz, 1H), 7.81-7.63 (m, 1H), 7.46 (s, 1H), 7.26 (br s, 1H), 6.96 (dd, J=1.6, 7.2 Hz, 1H), 6.90 (br s, 1H), 4.00-3.92 (m, 2H), 3.87 (s, 3H), 3.65-3.57 (m, 1H), 3.32-3.23 (m, 1H), 2.99 (s, 3H), 2.93 (s, 3H), 2.80-2.72 (m, 1H), 2.53 (s, 3H), 2.22 (s, 3H), 2.02-1.92 (m, 1H), 1.91-1.79 (m, 1H); ¹H NMR (400 MHz, CD3OD) δ 8.50 (d, J=7.2 Hz, 1H), 7.66 (d, J=0.8 Hz, 1H), 7.45 (s, 1H), 7.28 (br s, 1H), 6.96 (dd, J=2.0, 7.2 Hz, 1H), 6.90 (br s, 1H), 4.10-3.97 (m, 2H), 3.90 (s, 3H), 3.59-3.48 (m, 1H), 3.45-3.37 (m, 1H), 3.11 (s, 3H), 3.01 (s, 3H), 3.00-2.93 (m, 1H), 2.59 (s, 3H), 2.35 (s, 3H), 2.14-1.96 (m, 2H)

### Example 109: N,4-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide

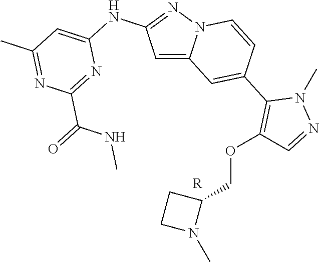

The title compound (39.7 mg, 5 steps, 7%) was prepared as described in Example 36, except methyl 4-chloro-6-methylpyrimidine-2-carboxylate was used instead of methyl 6-bromonicotinate in Step A and methylamine hydrochloride was used instead of 3-amino-2,2-dimethylpropanenitrile in Step C. MS (ESI): mass calcd. for C₂₃H₂₇N₉O₂, 461.2. m/z found, 462.2 [M+H]⁺. ¹H NMR (400 MHz, DMSO-d6) δ 10.64 (br s, 1H), 8.65 (d, J=7.2 Hz, 1H), 8.59-8.50 (m, 1H), 7.70 (d, J=0.8 Hz, 1H), 7.46 (s, 1H), 7.29 (br s, 1H), 7.09-6.85 (m, 2H), 3.98-3.91 (m, 2H), 3.88 (s, 3H), 3.27-3.17 (m, 2H), 2.82 (d, J=4.8 Hz, 3H), 2.75-2.66 (m, 1H), 2.43 (s, 3H), 2.19 (s, 3H), 1.98-1.89 (m, 1H), 1.88-1.78 (m, 1H)

### Example 110: N,N,4-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide

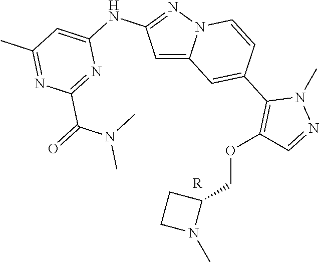

The title compound (53.9 mg, 5 steps, 9%) was prepared as described in Example 36, except methyl 4-chloro-6-methylpyrimidine-2-carboxylate was used instead of methyl 6-bromonicotinate in Step A and dimethylamine hydrochloride was used instead of 3-amino-2,2-dimethylpropanenitrile in Step C. MS (ESI): mass calcd. for C₂₄H₂₉N₉O₂, 475.2. m/z found, 476.3 [M+H]⁺. ¹H NMR (400 MHz, DMSO-d6) δ 10.56 (br s, 1H), 8.65 (d, J=7.6 Hz, 1H), 7.73 (d, J=0.8 Hz, 1H), 7.45 (s, 1H), 7.16 (br s, 1H), 6.96 (dd, J=2.0, 7.2 Hz, 1H), 6.79 (br s, 1H), 3.97-3.90 (m, 2H), 3.87 (s, 3H), 3.27-3.19 (m, 2H), 3.00 (s, 3H), 2.84 (s, 3H), 2.74-2.66 (m, 1H), 2.38 (s, 3H), 2.19 (s, 3H), 1.98-1.88 (m, 1H), 1.88-1.78 (m, 1H)

### Example 111: N-(2-hydroxy-2-methyl-propyl)-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide

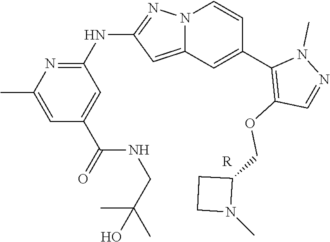

The title compound (55.1 mg, 5 steps, 24%) was prepared as described in Example 36, except methyl 2-chloro-6-methylisonicotinate was used instead of methyl 6-bromonicotinate in Step A and 1-amino-2-methylpropan-2-ol was used instead of 3-amino-2,2-dimethylpropanenitrile in Step C. MS (ESI): mass calcd. for C₂₇H₃₄N₈O₃, 518.3. m/z found, 519.3 [M+H]⁺. ¹H NMR (400 MHz, DMSO-d6) δ 9.94 (s, 1H), 8.57 (d, J=7.2 Hz, 1H), 8.31 (t, J=6.0 Hz, 1H), 7.67 (d, J=0.8 Hz, 1H), 7.47 (s, 1H), 7.46 (s, 1H), 7.06 (s, 1H), 6.93 (s, 1H), 6.90 (dd, J=1.6, 7.2 Hz, 1H), 4.59 (s, 1H), 4.01 (d, J=5.2 Hz, 2H), 3.87 (s, 3H), 3.59-3.42 (m, 2H), 3.25 (d, J=6.4 Hz, 2H), 3.00-2.86 (m, 1H), 2.48 (s, 3H), 2.31 (s, 3H), 2.06-1.96 (m, 1H), 1.96-1.87 (m, 1H), 1.12 (s, 6H)

### Example 112: N-isopropyl-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide

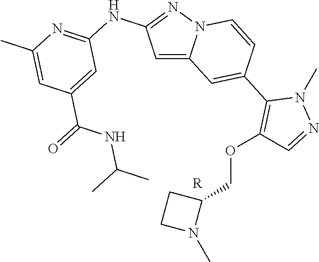

The title compound (41.9 mg, 5 steps, 21%) was prepared as described in Example 36, except propan-2-amine was used instead of 3-amino-2,2-dimethylpropanenitrile in Step C. MS (ESI): mass calcd. for C₂₆H₃₂N₈O₂, 488.3. m/z found, 489.3 [M+H]⁺. ¹H NMR (400 MHz, CDCl₃) δ 8.36 (d, J=7.2 Hz, 1H), 7.57 (s, 1H), 7.45 (s, 1H), 7.43 (br. s., 1H), 7.37 (s, 1H), 6.93 (s, 1H), 6.81 (dd, J=2.0, 7.2 Hz, 1H), 6.61 (s, 1H), 6.08 (d, J=7.6 Hz, 1H), 4.34-4.26 (m, 1H), 4.26-4.18 (m, 1H), 4.04 (dd, J=3.6, 10.4 Hz, 1H), 3.90 (s, 3H), 3.72-3.59 (m, 2H), 3.16-3.04 (m, 1H), 2.53 (s, 3H), 2.47 (s, 3H), 2.20-2.13 (m, 2H), 1.29 (d, J=6.4 Hz, 6H)

### Example 113: N-(2-hydroxy-2-methyl-propyl)-4-methyl-6-[[5-[2-methyl-4-[[(2R)-1-methylazetidin-2-yl]methoxy]pyrazol-3-yl]pyrazolo[1,5-a]pyridin-2-yl]amino]pyridine-2-carboxamide

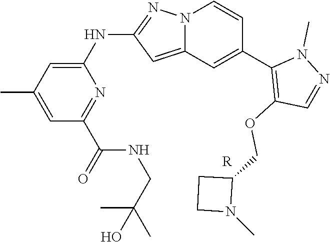

The title compound (50 mg, 5 steps, 0.7%) was prepared as described in Example 111, except methyl 6-chloro-4-methylpicolinate was used instead of methyl 6-bromonicotinate in Step A. MS (ESI): mass calcd. for C₂₇H₃₄N₈O₃, 518.3. m/z found, 519.1 [M+H]⁺. ¹H NMR (400 MHz, DMSO-d6) δ 10.05 (s, 1H), 8.61 (d, J=7.2 Hz, 1H), 8.32 (t, J=5.6 Hz, 1H), 7.58 (s, 1H), 7.46 (s, 1H), 7.34 (s, 1H), 7.22 (s, 1H), 7.00-6.79 (m, 2H), 4.87 (s, 1H), 3.97-3.90 (m, 2H), 3.86 (s, 3H), 3.30 (d, J=6.0 Hz, 2H), 3.27-3.19 (m, 2H), 2.75-2.64 (m, 1H), 2.35 (s, 3H), 2.18 (s, 3H), 1.98-1.89 (m, 1H), 1.88-1.78 (m, 1H), 1.18 (s, 6H); ¹H NMR (400 MHz, CDCl₃) δ 8.38 (t, J=6.0 Hz, 1H), 8.34 (d, J=7.2 Hz, 1H), 7.58 (s, 1H), 7.51 (d, J=0.8 Hz, 1H), 7.35 (s, 1H), 7.30 (s, 1H), 7.23 (s, 1H), 6.78 (dd, J=2.0, 7.2 Hz, 1H), 6.68 (s, 1H), 4.00 (d, J=5.6 Hz, 2H), 3.91 (s, 3H), 3.55-3.45 (m, 2H), 3.44-3.38 (m, 1H), 3.37-3.32 (m, 1H), 2.85-2.79 (m, 1H), 2.41 (s, 3H), 2.30 (s, 3H), 2.01-1.92 (m, 2H), 1.33 (s, 3H), 1.32 (s, 3H).

### Example 114: N-[5-[2-methyl-4-[[(2R)-1-methylazetidin-2-yl]methoxy]pyrazol-3-yl]pyrazolo[1,5-a]pyridin-2-yl]-7,8-dihydro-5H-pyrano[4,3-b]pyridin-2-amine

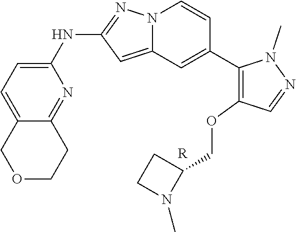

The title compound (68.4 mg, 3 steps, 45%) was made as described in Example 17, except 2-chloro-7,8-dihydro-5H-pyrano[4,3-b]pyridine (1.1 equiv.) was used instead of 3,4-dimethylpyridazine in Step A. MS (ESI): mass calcd. for C₂₄H₂₇N₇O₂, 445.2. m/z found, 446.1 [M+H]⁺. ¹H NMR (400 MHz, DMSO-d6) δ 9.71 (s, 1H), 8.55 (d, J=7.2 Hz, 1H), 7.62 (d, J=0.8 Hz, 1H), 7.44 (s, 1H), 7.32 (d, J=8.4 Hz, 1H), 7.19 (d, J=8.8 Hz, 1H), 6.88-6.84 (m, 2H), 4.61 (s, 2H), 3.96 (t, J=6.0 Hz, 2H), 3.94-3.90 (m, 2H), 3.86 (s, 3H), 3.28-3.16 (m, 2H), 2.81 (t, J=5.6 Hz, 2H), 2.74-2.66 (m, 1H), 2.19 (s, 3H), 1.97-1.89 (m, 1H), 1.89-1.78 (m, 1H)

### Example 115: N-(2-methoxyethyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide

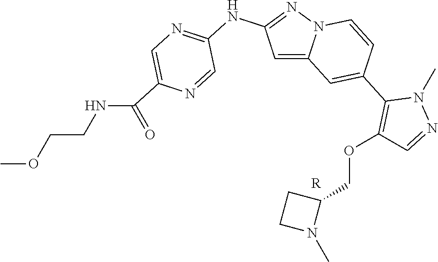

The title compound (87 mg, 5 steps, 25%) was made as described in Example 36, except methyl 5-chloropyrazine-2-carboxylate (1.1 equiv.) was used instead of methyl 6-bromonicotinate in Step A and 2-methoxyethanamine (1.2 equiv.) was used instead of 3-amino-2,2-dimethylpropanenitrile in Step C. MS (ESI): mass calcd. for C₂₄H₂₉N₉O₃, 491.2. m/z found, 492.2 [M+H]⁺. ¹H NMR (400 MHz, CDCl₃) δ 9.02 (d, J=1.2 Hz, 1H), 8.65 (d, J=1.2 Hz, 1H), 8.37 (d, J=7.2 Hz, 1H), 8.20 (s, 1H), 7.93 (t, J=5.6 Hz, 1H), 7.49 (d, J=0.8 Hz, 1H), 7.35 (s, 1H), 6.88 (dd, J=1.6, 6.8 Hz, 1H), 6.76 (s, 1H), 3.99 (d, J=5.2 Hz, 2H), 3.92 (s, 3H), 3.71-3.65 (m, 2H), 3.62-3.56 (m, 2H), 3.45-3.39 (m, 4H), 3.39-3.30 (m, 1H), 2.87-2.79 (m, 1H), 2.33 (s, 3H), 2.07-1.94 (m, 2H)

### Example 116: 5-[2-methyl-4-[[(2R)-1-methylazetidin-2-yl]methoxy]pyrazol-3-yl]-N-[6-methyl-2-(3-pyridyl)pyrimidin-4-yl]pyrazolo[1,5-a]pyridin-2-amine

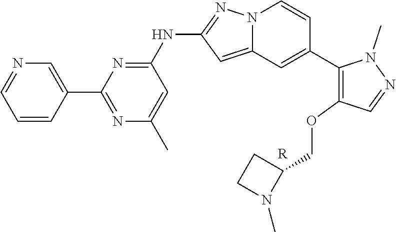

The title compound (61.8 mg, 3 steps, 35%) was prepared as described in Example 17, except 4-chloro-6-methyl-2-(pyridin-3-yl)pyrimidine was used instead of 3,4-dimethylpyridazine in Step A. MS (ESI): mass calcd. for C₂₆H₂₇N₉O, 481.2. m/z found, 482.2 [M+H]⁺. ¹H NMR (400 MHz, CDCl₃) δ 9.68-9.65 (m, 1H), 8.72-8.70 (m, 1H), 8.70-8.68 (m, 1H), 8.38 (d, J=6.8 Hz, 1H), 7.81 (s, 1H), 7.53 (d, J=0.8 Hz, 1H), 7.45-7.39 (m, 1H), 7.36 (s, 1H), 7.27 (s, 1H), 6.89 (dd, J=2.0, 7.2 Hz, 1H), 6.79 (s, 1H), 3.99 (d, J=5.6 Hz, 2H), 3.94 (s, 3H), 3.45-3.39 (m, 1H), 3.38-3.30 (m, 1H), 2.86-2.78 (m, 1H), 2.54 (s, 3H), 2.34 (s, 3H), 2.05-1.95 (m, 2H)

### Example 117: 5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine

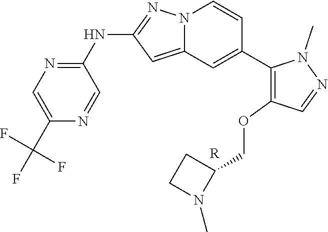

Step A. Tert-butyl (R)-2-(((1-methyl-5-(2-((5-(trifluoromethyl)pyrazin-2-yl)amino)pyrazolo[1,5-a]pyridin-5-yl)-1H-pyrazol-4-yl)oxy)methyl)azetidine-1-carboxylate. A mixture consisting of Intermediate 6 (500 mg, 1.3 mmol, 1 equiv.), 2-chloro-5-(trifluoromethyl)pyrazine (458 mg, 2.5 mmol, 2 equiv.), Brettphos-Pd-G3 (114 mg, 0.13 mmol, 0.1 equiv.) and Brettphos (135 mg, 0.251 mmol, 0.2 equiv.), CsF (572 mg, 3.77 mmol, 3 equiv.), and 1,4-dioxane (10 mL) was sparged with argon for 2 minutes and stirred overnight at 80° C. The mixture was concentrated to dryness under reduced pressure to afford the crude product which was purified by FCC (eluent: petroleum ether:EtOAc=1:0 to 1:4) to give the title compound (480 mg, overall yield 66%) as a yellow oil.

Step B. (R)-5-(4-(azetidin-2-ylmethoxy)-1-methyl-1H-pyrazol-5-yl)-N-(5-(trifluoromethyl)pyrazin-2-yl)pyrazolo[1,5-a]pyridin-2-amine. A solution consisting of tert-butyl (R)-2-(((1-methyl-5-(2-((5-(trifluoromethyl)pyrazin-2-yl)amino)pyrazolo[1,5-a]pyridin-5-yl)-1H-pyrazol-4-yl)oxy)methyl)azetidine-1-carboxylate (470 mg, 0.86 mmol, 1 equiv.) and 1,4-dioxane/HCl (10 mL) was stirred at room-temperature for 30 min. The mixture was concentrated under reduced pressure then H₂O (20 mL) was added to the solution and the mixture was neutralized with aqueous NaHCO₃. The mixture was extracted with EtOAc (30 mL×3) and the combined organic layers were dried over Na₂SO₄ then filtered and concentrated under reduce pressure to afford the title compound (500 mg, crude) as a light yellow oil.

Step C. 5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine. A solution consisting of (R)-5-(4-(azetidin-2-ylmethoxy)-1-methyl-1H-pyrazol-5-yl)-N-(5-(trifluoromethyl)pyrazin-2-yl)pyrazolo[1,5-a]pyridin-2-amine (150 mg, 0.34 mmol, 1 equiv.), paraformaldehyde (32.9 mg, 0.37 mmol, 1.08 equiv.), NaHCO₃ (42.5 mg, 0.51 mmol, 1.5 equiv.) and MeOH (10 mL) was stirred at room-temperature for 10 min. To the solution was added NaBH₃CN (31.8 mg, 0.51 mmol, 1.5 equiv.) and the resultant mixture was stirred at room-temperature for 40 min. The pH of the reaction mixture was adjusted to 6 with 1M HCl. The reaction was co-treated with a previous reaction. The crude product was purified by preparative HPLC using Phenomenex Gemini-NX C18 75×30 mm×3 μm column (eluent: 45% to 75% (v/v) CH₃CN and H₂O with 0.04% NH₃H₂O+10 mM NH₄HCO₃) to afford pure product. The product was suspended in water (10 mL), the mixture frozen using dry ice/ethanol, and then lyophilized to dryness to afford the title compound (52 mg, 25%) as a white solid. MS (ESI): mass calcd. for C₂₁H₂₁F₃N₈O, 458.2. m/z found, 459.1 [M+H]⁺. ¹H NMR (400 MHz, CDCl₃) δ 8.72-8.67 (m, 1H), 8.48 (s, 1H), 8.29 (d, J=7.2 Hz, 1H), 7.75 (s, 1H), 7.44 (d, J=0.8 Hz, 1H), 7.28 (s, 1H), 6.83 (dd, J=1.6, 6.8 Hz, 1H), 6.69 (s, 1H), 3.91 (d, J=5.2 Hz, 2H), 3.85 (s, 3H), 3.39-3.32 (m, 1H), 3.30-3.22 (m, 1H), 2.80-2.71 (m, 1H), 2.26 (s, 3H), 1.99-1.90 (m, 2H) 19F NMR (376 MHz, CDCl₃) δ −66.69 (s, 3F)

### Example 118: 5-[2-methyl-4-[[(2R)-1-methylazetidin-2-yl]methoxy]pyrazol-3-yl]-N-[2-(trifluoromethyl)pyrimidin-4-yl]pyrazolo[1,5-a]pyridin-2-amine

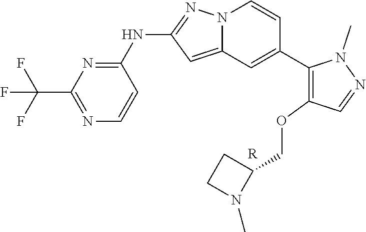

The title compound (8.4 mg, 3 steps, 7%) was prepared as detailed in previous experiments (Example 36 Step A then Example 17 Steps B-C), except 4-chloro-2-(trifluoromethyl)pyrimidine (2 equiv.) was used instead of methyl-6-bromonicoate in Step A of Example 36 and cesium carbonate (2 equiv.) was used instead of cesium fluoride in Step A of Example 36. In Step C of Example 17 NaHCO₃ (3 equiv.) was added to the reaction mixture. MS (ESI): mass calcd. for C₂₁H₂₁F₃N₈O, 458.2. m/z found, 459.1 [M+H]⁺. ¹H NMR (400 MHz, CDCl₃) δ 8.55 (d, J=6.0 Hz, 1H), 8.40 (d, J=7.2 Hz, 1H), 8.04 (s, 1H), 7.60-7.44 (m, 2H), 7.35 (s, 1H), 6.93-6.89 (m, 1H), 6.64 (br. s., 1H), 3.99 (d, J=5.6 Hz, 2H), 3.92 (s, 3H), 3.46-3.40 (m, 1H), 3.39-3.30 (m, 1H), 2.87-2.79 (m, 1H), 2.34 (s, 3H), 2.05-1.97 (m, 2H) 19F NMR (376 MHz, CDCl₃) δ −71.14 (s, 3F)

### Example 119: N-(2-methoxypyrimidin-4-yl)-5-[2-methyl-4-[[(2R)-1-methylazetidin-2-yl]methoxy]pyrazol-3-yl]pyrazolo[1,5-a]pyridin-2-amine

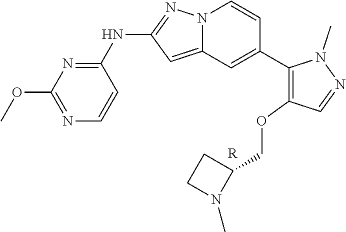

The title compound (54.3 mg, 3 steps, 17%) was prepared as detailed in Example 117, except 4-chloro-2-methoxypyrimidine (1.3 equiv.) was used instead of 2-chloro-5-(trifluoromethyl)pyrazine in Step A and cesium carbonate (3 equiv.) was used instead of cesium fluoride in Step A. In Step B TFA/DCM (1:2) was used instead of 1,4-dioxane/HCl. MS (ESI): mass calcd. for C₂₁H₂₄N₈O₂, 420.2. m/z found, 421.1 [M+H]⁺. ¹H NMR (400 MHz, DMSO-d6) δ 10.49 (s, 1H), 8.62 (d, J=7.2 Hz, 1H), 8.17 (d, J=6.0 Hz, 1H), 7.73 (d, J=0.8 Hz, 1H), 7.45 (s, 1H), 7.07-6.91 (m, 2H), 6.85 (br s, 1H), 3.98-3.89 (m, 5H), 3.87 (s, 3H), 3.29-3.19 (m, 2H), 2.75-2.66 (m, 1H), 2.19 (s, 3H), 2.00-1.89 (m, 1H), 1.88-1.79 (m, 1H)

### Example 120: N,2-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide

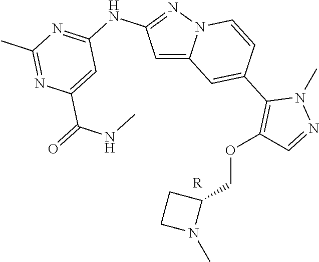

Step A. (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)-2-methylpyrimidine-4-carboxylic acid. A mixture consisting of Intermediate 6 (500 mg, 1.25 mmol, 1 equiv.), ethyl 6-chloro-2-methylpyrimidine-4-carboxylate (252 mg, 1.26 mmol, 1 equiv.), Brettphos-Pd-G3 (114 mg, 0.13 mmol, 0.1 equiv.), Brettphos (135 mg, 0.25 mmol, 0.2 equiv.), Cs₂CO₃ (1227 mg, 3.77 mmol, 3 equiv.), 1,4-dioxane (8 mL) was stirred overnight at 50° C. under an argon atmosphere. The reaction mixture was directly concentrated to afford the crude product, which was purified by FCC (eluent: ethyl acetate:ethanol=1:0 to 1:1) to afford the title compound (350 mg, 50%) as a yellow solid.

Step B. Tert-butyl (R)-2-(((1-methyl-5-(2-((2-methyl-6-(methylcarbamoyl)pyrimidin-4-yl)amino)pyrazolo[1,5-a]pyridin-5-yl)-1H-pyrazol-4-yl)oxy)methyl)azetidine-1-carboxylate. To a mixture consisting of (R)-6-((5-(4-((1-(tert-butoxycarbonyl)azetidin-2-yl)methoxy)-1-methyl-1H-pyrazol-5-yl)pyrazolo[1,5-a]pyridin-2-yl)amino)-2-methylpyrimidine-4-carboxylic acid (320 mg, 0.60 mmol, 1 equiv.), MeNH₂HCl (44.5 mg, 0.66 mmol, 1.1 equiv.), HATU (273 mg, 0.72 mmol, 1.2 equiv.), and DMF (10 mL) stirred for 1.5 hr at room temperature was added DIEA (155 mg, 1.20 mmol, 2 equiv.). To the reaction mixture was added H2O (50 mL) and the suspension was isolated via filtration. The filter cake was washed with water (10 mL×2) before drying under reduced pressure to afford the title compound (280 mg, 81%).

Step C. (R)-6-((5-(4-(azetidin-2-ylmethoxy)-1-methyl-1H-pyrazol-5-yl)pyrazolo[1,5-a]pyridin-2-yl)amino)-N,2-dimethylpyrimidine-4-carboxamide. To a mixture of (R)-tert-butyl 2-(((1-methyl-5-(2-((2-methyl-6-(methylcarbamoyl)pyrimidin-4-yl)amino)pyrazolo[1,5-a]pyridin-5-yl)-1H-pyrazol-4-yl)oxy)methyl)azetidine-1-carboxylate (280 mg, 0.511 mmol) and DCM (6 mL) was added TFA (3 mL). The reaction mixture was directly concentrated to afford the title compound (500 mg, crude) without further purification.

Step D. N,2-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide. A solution consisting of (R)-6-((5-(4-(azetidin-2-ylmethoxy)-1-methyl-1H-pyrazol-5-yl)pyrazolo[1,5-a]pyridin-2-yl)amino)-N,2-dimethylpyrimidine-4-carboxamide (470 mg, 1.05 mmol, 1 equiv.), paraformaldehyde (94.6 mg, 1.05 mmol, 1 equiv.), NaBH₃CN (132 mg, 2.10 mmol, 2 equiv.) and MeOH (5 mL) was stirred for 1 hr at room temperature. The reaction mixture was purified by SFC over DAICEL CHIRALCEL OD 250 mm×30 mm, 10 μm (eluent: 45% to 45% (v/v) supercritical CO₂ in EtOH and H₂O with 0.1% NH₃). The pure fractions were collected and the volatiles were removed under reduced pressure. The product was suspended in water (10 mL), the mixture frozen using dry ice/acetone, and then lyophilized to dryness to afford the title compound (19 mg, 4%) as a white solid. MS (ESI): mass calcd. for C₂₃H₂₇N₉O₂, 461.2. m/z found, 462.2 [M+H]⁺. ¹H NMR (400 MHz, DMSO-d6) δ 10.68 (s, 1H), 8.73-8.60 (m, 2H), 7.75 (d, J=1.2 Hz, 1H), 7.60 (br s, 1H), 7.47 (s, 1H), 7.14-6.85 (m, 2H), 4.06-3.92 (m, 2H), 3.88 (s, 3H), 3.56-3.40 (m, 2H), 2.95-2.84 (m, 1H), 2.82 (d, J=4.8 Hz, 3H), 2.59 (s, 3H), 2.29 (s, 3H), 2.07-1.85 (m, 2H) 1H NMR (400 MHz, CD3OD) δ 8.51 (d, J=7.2 Hz, 1H), 7.75-7.63 (m, 2H), 7.47 (s, 1H), 7.00 (s, 1H), 6.96 (dd, J=2.0, 7.2 Hz, 1H), 4.18-4.02 (m, 2H), 3.91 (s, 3H), 3.85-3.74 (m, 1H), 3.61-3.50 (m, 1H), 3.24-3.09 (m, 1H), 2.96 (s, 3H), 2.62 (s, 3H), 2.47 (s, 3H), 2.24-2.07 (m, 2H)

### Example 121: N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine

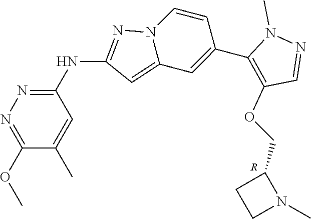

Step A. Tert-butyl (R)-2-(((5-(2-((6-methoxy-5-methylpyridazin-3-yl)amino)pyrazolo[1,5-a]pyridin-5-yl)-1-methyl-1H-pyrazol-4-yl)oxy)methyl)azetidine-1-carboxylate. A mixture consisting of Intermediate 6 (600 mg, 1.51 mmol, 1 equiv.), 6-chloro-3-methoxy-4-methylpyridazine (310 mg, 1.96 mmol, 1.3 equiv.), Xantphos (174 mg, 0.301 mmol, 0.2 equiv.), Pd(OAc)₂ (33.8 mg, 0.151 mmol, 0.1 equiv.), Cs₂CO₃ (1472 mg, 4.52 mmol, 3 equiv.), 1,4-dioxane (15 mL) was stirred overnight at 100° C. under a nitrogen atmosphere. The reaction mixture was directly concentrated to afford the crude product, which was purified by FCC (eluent: PE:EA=1:0 to 0:1) to afford the title compound (700 mg, 89%) as a yellow solid.

Step B. (R)-5-(4-(azetidin-2-ylmethoxy)-1-methyl-1H-pyrazol-5-yl)-N-(6-methoxy-5-methylpyridazin-3-yl)pyrazolo[1,5-a]pyridin-2-amine. A solution consisting of (R)-tert-butyl 2-(((5-(2-((6-methoxy-5-methylpyridazin-3-yl)amino)pyrazolo[1,5-a]pyridin-5-yl)-1-methyl-1H-pyrazol-4-yl)oxy)methyl)azetidine-1-carboxylate (700 mg, 1.35 mmol, 1 equiv.), TFA (5 mL), and DCM (10 mL) was stirred for 1 hr at room temperature. The reaction mixture was directly concentrated to afford the title compound (1 g, crude) as a yellow solid.

Step C. N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine. A solution consisting of (R)-5-(4-(azetidin-2-ylmethoxy)-1-methyl-1H-pyrazol-5-yl)-N-(6-methoxy-5-methylpyridazin-3-yl)pyrazolo[1,5-a]pyridin-2-amine (950 mg, 2.26 mmol, 1 equiv.), paraformaldehyde (224 mg, 2.49 mmol, 1.1 equiv.), NaBH₃CN (284 mg, 4.52 mmol, 2 equiv.) and MeOH (5 mL) was stirred for 1 hr at room temperature. The reaction mixture was purified by preparative HPLC using a Boston Prime C18 150×30 mm×5 μm column (eluent: 35% to 65% (v/v) CH₃CN and H₂O with 0.05% NH₃ and 10 mM NH₄HCO₃) to afford pure product. The product was suspended in water (10 mL), the mixture frozen using dry ice/ethanol, and then lyophilized to dryness to afford the title compound (96.6 mg, 10%) as a white solid. MS (ESI): mass calcd. for C₂₂H₂₆N₈O₂, 434.2. m/z found, 435.1 [M+H]⁺. ¹H NMR (400 MHz, DMSO-d6) δ 9.82 (s, 1H), 8.58 (d, J=7.2 Hz, 1H), 7.65 (d, J=0.8 Hz, 1H), 7.45 (s, 1H), 7.37 (s, 1H), 6.94 (s, 1H), 6.89 (dd, J=1.6, 7.2 Hz, 1H), 3.97 (s, 3H), 3.95-3.90 (m, 2H), 3.86 (s, 3H), 3.28-3.18 (m, 2H), 2.77-2.64 (m, 1H), 2.20 (s, 3H), 2.17 (s, 3H), 1.98-1.89 (m, 1H), 1.89-1.78 (m, 1H)

### Example 122: 5-[2-methyl-4-[[(2R)-1-methylazetidin-2-yl]methoxy]pyrazol-3-yl]-N-[2-methyl-6-(trifluoromethyl)pyrimidin-4-yl]pyrazolo[1,5-a]pyridin-2-amine

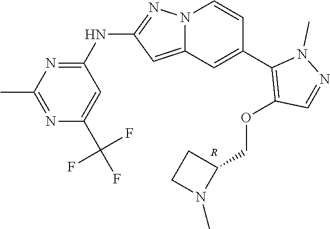

The title compound (86.3 mg, 18%) was prepared as described in Example 120, except 4-chloro-2-methyl-6-(trifluoromethyl)pyrimidine (1 equiv.) was used instead of ethyl 6-chloro-2-methylpyrimidine-4-carboxylate. MS (ESI): mass calcd. for C₂₂H₂₃F₃N₈O, 472.2. m/z found, 473.2 [M+H]⁺. ¹H NMR (400 MHz, DMSO-d6) δ 10.96 (s, 1H), 8.68 (d, J=6.8 Hz, 1H), 8.11-6.51 (m, 5H), 3.99-3.91 (m, 2H), 3.88 (s, 3H), 3.28-3.18 (m, 2H), 2.75-2.66 (m, 1H), 2.60 (s, 3H), 2.20 (s, 3H), 1.98-1.89 (m, 1H), 1.89-1.79 (m, 1H) 1H NMR (400 MHz, DMSO-d6, t=80° C.) δ 10.59 (s, 1H), 8.59 (d, J=7.2 Hz, 1H), 7.70 (s, 1H), 7.54 (s, 1H), 7.40 (s, 1H), 6.99-6.94 (m, 1H), 6.91 (s, 1H), 3.93 (d, J=5.6 Hz, 2H), 3.85 (s, 3H), 3.29-3.18 (m, 2H), 2.76-2.67 (m, 1H), 2.58 (s, 3H), 2.20 (s, 3H), 1.99-1.91 (m, 1H), 1.89-1.79 (m, 1H) 19F NMR (376 MHz, DMSO-d6) δ −69.32 (s, 3F)

### Example 123: 5-[2-methyl-4-[[(2R)-1-methylazetidin-2-yl]methoxy]pyrazol-3-yl]-N-[6-(trifluoromethyl)pyrimidin-4-yl]pyrazolo[1,5-a]pyridin-2-amine

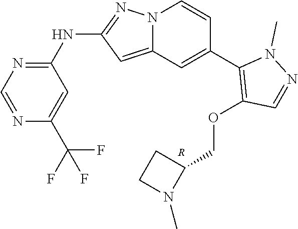

The title compound (45.3 mg, 3 steps, 19%) was prepared as described in Example 121, except 2-chloro-5-fluoropyrimidine (1.1 equiv.) was used instead of 6-chloro-3-methoxy-4-methylpyridazine in Step A. MS (ESI): mass calcd. for C₂₁H₂₁F₃N₈O, 458.2. m/z found, 459.2 [M+H]⁺. ¹H NMR (400 MHz, CDCl₃) δ 8.89 (s, 1H), 8.53-8.35 (m, 2H), 7.79 (br. s., 1H), 7.51 (s, 1H), 7.35 (s, 1H), 6.92 (dd, J=1.6, 7.2 Hz, 1H), 6.60 (br. s., 1H), 3.99 (d, J=5.2 Hz, 2H), 3.92 (s, 3H), 3.46-3.39 (m, 1H), 3.38-3.30 (m, 1H), 2.87-2.78 (m, 1H), 2.33 (s, 3H), 2.07-1.96 (m, 2H) 19F NMR (376 MHz, CDCl₃) δ −70.38 (s, 3F)

### Example 124: 5-[2-methyl-4-[[(2R)-1-methylazetidin-2-yl]methoxy]pyrazol-3-yl]-N-(1-methyltriazol-4-yl)pyrazolo[1,5-a]pyridin-2-amine

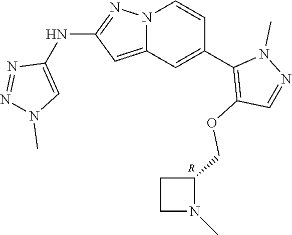

The title compound (290 mg, 3 steps, 8%) was prepared as described in Example 2, except 4-bromo-1-methyl-1H-1,2,3-triazole was used instead of 4-chloro-2,6-dimethylpyrimidine in Step A. MS (ESI): mass calcd. for C₁₉H₂₃N₉O, 393.2. m/z found, 394.1 [M+H]⁺. ¹H NMR (400 MHz, CDCl₃) δ 8.31 (d, J=7.2 Hz, 1H), 7.87 (s, 1H), 7.44 (s, 1H), 7.36 (d, J=1.2 Hz, 1H), 7.34 (s, 1H), 6.73 (dd, J=2.0, 7.2 Hz, 1H), 6.05 (s, 1H), 4.11 (s, 3H), 4.03-3.95 (m, 2H), 3.90 (s, 3H), 3.48-3.42 (m, 1H), 3.41-3.32 (m, 1H), 2.90-2.81 (m, 1H), 2.34 (s, 3H), 2.07-1.96 (m, 2H)

### Example 125: N-[5-[2-methyl-4-[[(2R)-1-methylazetidin-2-yl]methoxy]pyrazol-3-yl]pyrazolo[1,5-a]pyridin-2-yl]-1,6-naphthyridin-2-amine

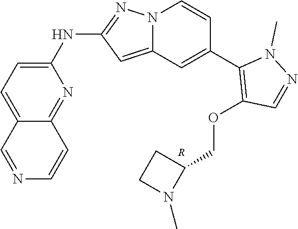

The title compound (110 mg, 3 steps, 36%) was prepared as described in Example 2, except 2-chloro-1,6-naphthyridine was used instead of 4-chloro-2,6-dimethylpyrimidine in Step A and Brettphos-Pd-G3/Brettphos were used instead of t-BuBrettphos in Step A. MS (ESI): mass calcd. for C₂₄H₂₄N₈O, 440.2. m/z found, 441.1 [M+H]⁺. ¹H NMR (400 MHz, CDCl₃) δ 9.01 (s, 1H), 8.62 (d, J=6.0 Hz, 1H), 8.37 (d, J=7.2 Hz, 1H), 8.21 (br. s., 1H), 8.08 (d, J=8.8 Hz, 1H), 7.67 (d, J=5.6 Hz, 1H), 7.54 (d, J=0.8 Hz, 1H), 7.36 (s, 1H), 7.32 (d, J=8.8 Hz, 1H), 7.21 (s, 1H), 6.89 (dd, J=1.6, 6.8 Hz, 1H), 3.99 (d, J=5.6 Hz, 2H), 3.93 (s, 3H), 3.47-3.39 (m, 1H), 3.38-3.30 (m, 1H), 2.88-2.78 (m, 1H), 2.34 (s, 3H), 2.05-1.96 (m, 2H)

### Example 126: N-methyl-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide

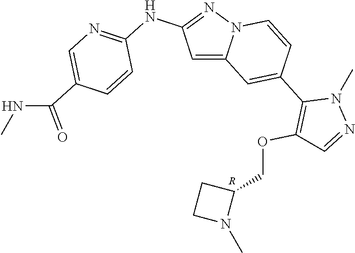

The title compound (111.2 mg, 3 steps, 39%) was prepared as described in Example 2, except 6-chloro-N-methylnicotinamide was used instead of 4-chloro-2,6-dimethylpyrimidine in Step A and Brettphos-Pd-G3/Brettphos were used instead of t-BuBrettphos in Step A. MS (ESI): mass calcd. for C₂₃H₂₆N₈O₂, 446.2. m/z found, 447.1 [M+H]⁺. ¹H NMR (400 MHz, CDCl₃) δ 8.67 (d, J=2.0 Hz, 1H), 8.36 (d, J=7.2 Hz, 1H), 8.04 (dd, J=2.0, 8.8 Hz, 1H), 7.77 (s, 1H), 7.48-7.41 (m, 2H), 7.34 (s, 1H), 6.84 (dd, J=2.0, 7.2 Hz, 1H), 6.60 (s, 1H), 6.18-6.12 (m, 1H), 3.97 (d, J=5.6 Hz, 2H), 3.91 (s, 3H), 3.45-3.38 (m, 1H), 3.37-3.28 (m, 1H), 3.03 (d, J=4.8 Hz, 3H), 2.86-2.78 (m, 1H), 2.32 (s, 3H), 2.05-1.95 (m, 2H)

### Example 127: N-(6-methoxypyrimidin-4-yl)-5-[2-methyl-4-[[(2R)-1-methylazetidin-2-yl]methoxy]pyrazol-3-yl]pyrazolo[1,5-a]pyridin-2-amine

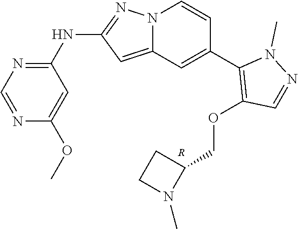

The title compound (74.2, 3 steps, 37%) was prepared as described in Example 2, except 4-chloro-6-methoxypyrimidine was used instead of 4-chloro-2,6-dimethylpyrimidine in Step A and Brettphos-G3 was used instead of t-BuBrettPhos in Step A. MS (ESI): mass calcd. for C₂₁H₂₄N₈O₂, 420.2. m/z found, 421.1 [M+H]⁺. ¹H NMR (400 MHz, CDCl₃) δ 8.48 (s, 1H), 8.38 (d, J=6.8 Hz, 1H), 7.87 (s, 1H), 7.45 (s, 1H), 7.35 (s, 1H), 6.90 (s, 1H), 6.85 (dd, J=2.0, 7.2 Hz, 1H), 6.43 (s, 1H), 3.99 (s, 3H), 3.97 (d, J=5.6 Hz, 2H), 3.91 (s, 3H), 3.44-3.38 (m, 1H), 3.36-3.29 (m, 1H), 2.86-2.78 (m, 1H), 2.32 (s, 3H), 2.05-1.93 (m, 2H)

### Example 128: N2-cyclopropyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine

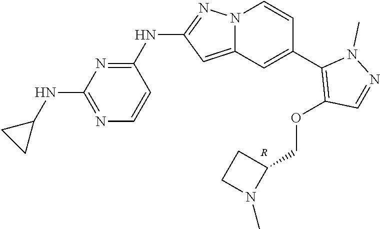

The title compound (67 mg, 3 steps, 25%) was prepared as described in Example 2, except 4-chloro-N-cyclopropylpyrimidin-2-amine was used instead of 4-chloro-2,6-dimethylpyrimidine in Step A and Brettphos-Pd-G3/Brettphos were used instead of t-BuBrettphos in Step A. MS (ESI): mass calcd. for C₂₃H₂₇N₉O, 445.2. m/z found, 446.2 [M+H]⁺. ¹H NMR (400 MHz, CDCl₃) δ 8.34 (d, J=7.2 Hz, 1H), 8.09 (d, J=5.6 Hz, 1H), 7.97 (br. s., 1H), 7.46 (d, J=0.8 Hz, 1H), 7.34 (s, 1H), 6.86 (br. s., 1H), 6.83 (dd, J=2.0, 7.2 Hz, 1H), 6.44 (d, J=5.2 Hz, 1H), 5.62 (br. s., 1H), 3.97 (d, J=5.2 Hz, 2H), 3.91 (s, 3H), 3.45-3.39 (m, 1H), 3.37-3.28 (m, 1H), 2.86-2.78 (m, 2H), 2.32 (s, 3H), 2.04-1.93 (m, 2H), 0.88-0.82 (m, 2H), 0.63-0.58 (m, 2H)

### Example 129: N2-cyclopentyl-6-methyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine

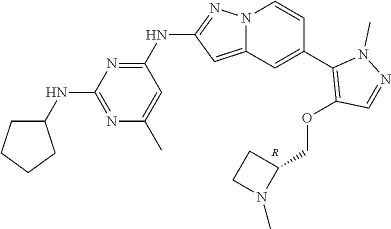

Step A. 2-chloro-N-cyclopentyl-6-methylpyrimidin-4-amine. A mixture consisting of 2,4-dichloro-6-methylpyrimidine (3.0 g, 18 mmol, 1 equiv.), cyclopentanamine (3.1 g, 37 mmol, 2 equiv.), TEA (3.7 g, 37 mmol, 2 equiv.) and THE (20 mL) was stirred at room-temperature overnight. The reaction mixture was concentrated under vacuum to give a residue, which was purified by FCC (eluent: EA:PE=0% to 15%) afford the title product (900 mg, 23%) as a colorless oil.

Step B. N2-cyclopentyl-6-methyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine. The title compound (103.5 mg, 3 steps, 42%) was prepared as described in Example 2, except 2-chloro-N-cyclopentyl-6-methylpyrimidin-4-amine was used instead of 4-chloro-2,6-dimethylpyrimidine in Step A. MS (ESI): mass calcd. for C₂₆H₃₃N₉O, 487.3. m/z found, 488.2 [M+H]⁺. ¹H NMR (400 MHz, DMSO-d6) δ 9.82 (s, 1H), 8.57 (d, J=7.2 Hz, 1H), 7.60 (s, 1H), 7.44 (s, 1H), 7.07 (br. s., 1H), 6.90 (dd, J=2.0, 7.2 Hz, 1H), 6.76-6.61 (m, 1H), 6.17 (s, 1H), 4.29-4.17 (m, 1H), 3.97-3.88 (m, 2H), 3.86 (s, 3H), 3.27-3.18 (m, 2H), 2.74-2.66 (m, 1H), 2.19 (s, 3H), 2.12 (s, 3H), 1.99-1.87 (m, 3H), 1.87-1.79 (m, 1H), 1.76-1.64 (m, 2H), 1.61-1.45 (m, 4H)

### Example 130: N-(2,6-dimethylpyrimidin-4-yl)-5-[2-ethyl-4-[[(2R)-1-methylazetidin-2-yl]methoxy]pyrazol-3-yl]pyrazolo[1,5-a]pyridin-2-amine

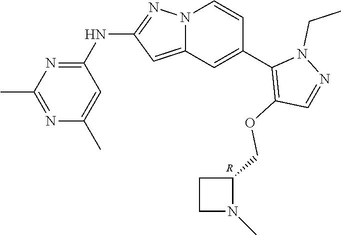

The title compound (170 mg, 3 steps, 35%) was prepared as described in Example 2, except (R)-tert-butyl 2-(((5-(2-aminopyrazolo[1,5-a]pyridin-5-yl)-1-ethyl-1H-pyrazol-4-yl)oxy)methyl)azetidine-1-carboxylate was used instead of Intermediate 6 in Step A and Brettphos-Pd-G3/Brettphos were used instead of t-BuBrettphos in Step A. The material (R)-tert-butyl 2-(((5-(2-aminopyrazolo[1,5-a]pyridin-5-yl)-1-ethyl-1H-pyrazol-4-yl)oxy)methyl)azetidine-1-carboxylate was prepared as described (1.1 g, 3 steps, 41%) in Intermediate 6, except 1-Ethyl-1H-pyrazol-4-ol was used instead of 1-methyl-1H-pyrazol-4-ol in Step A and DMF (10 mL) was used instead of DMSO in Step A. MS (ESI): mass calcd. for C₂₃H₂₈N₈O, 432.2. m/z found, 433.2 [M+H]⁺. ¹H NMR (400 MHz, CDCl₃) δ 8.40 (d, J=7.2 Hz, 1H), 7.59 (s, 1H), 7.45 (s, 1H), 7.38 (s, 1H), 7.06 (s, 1H), 6.81 (dd, J=2.0, 7.2 Hz, 1H), 6.58 (s, 1H), 4.19 (q, J=7.2 Hz, 2H), 3.97 (d, J=5.6 Hz, 2H), 3.44-3.37 (m, 1H), 3.36-3.25 (m, 1H), 2.85-2.75 (m, 1H), 2.61 (s, 3H), 2.45 (s, 3H), 2.31 (s, 3H), 2.05-1.95 (m, 2H), 1.41 (t, J=7.2 Hz, 3H)

### Example 131: N-(2,6-dimethylpyrimidin-4-yl)-5-[4-[[(2R)-1-methylazetidin-2-yl]methoxy]-2-(trideuteriomethyl)pyrazol-3-yl]pyrazolo[1,5-a]pyridin-2-amine

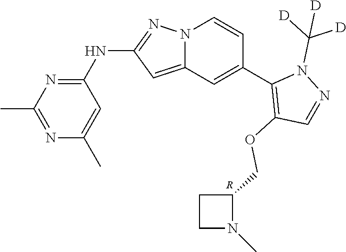

Step A. Tert-butyl (R)-2-(((1-(methyl-d3)-1H-pyrazol-4-yl)oxy)methyl)azetidine-1-carboxylate. A mixture consisting of (R)-tert-butyl 2-(((1H-pyrazol-4-yl)oxy)methyl)azetidine-1-carboxylate (2.3 g, 9.1 mmol, 1 equiv.), CD3I (2.6 g, 18 mmol, 2 equiv.), Cs₂CO₃ (5.9 g, 18 mmol, 2 equiv.) and DMF (30 mL) was stirred at room temperature for 2 hr. The reaction mixture was concentrated to dryness under reduced pressure to give the crude product, which was purified by FCC (eluent: petroleum ether:ethyl acetate)=100/0 to 50/50) to yield the title compound (1.25 g, 51%) as a yellow oil.

Step B. Tert-butyl (R)-2-(((5-bromo-1-(methyl-d3)-1H-pyrazol-4-yl)oxy)methyl)azetidine-1-carboxylate. A mixture of tert-butyl (R)-2-(((1-(methyl-d3)-1H-pyrazol-4-yl)oxy)methyl)azetidine-1-carboxylate (1.2 g, 4.4 mmol, 1 equiv.), NBS (830 mg, 4.66 mmol, 1.05 equiv.), and ACN (10 mL) was stirred at room-temperature for 1 hr. The reaction mixture was concentrated to dryness under reduced pressure to give the crude product (2 g, 129%), which was used for next reaction directly.

Step C. Tert-butyl (R)-2-(((5-(2-aminopyrazolo[1,5-a]pyridin-5-yl)-1-(methyl-d3)-1H-pyrazol-4-yl)oxy)methyl)azetidine-1-carboxylate. A mixture consisting of tert-butyl (R)-2-(((5-bromo-1-(methyl-d3)-1H-pyrazol-4-yl)oxy)methyl)azetidine-1-carboxylate (1.95 g, 5.58 mmol, 1 equiv.), 5-(4,4,5,5-tetramethyl-1,3,2-dioxaborolan-2-yl)pyrazolo[1,5-a]pyridin-2-amine (2.17 g, 8.38 mmol, 1.5 equiv.), Pd(dppf)Cl₂ (409 mg, 0.558 mmol, 0.1 equiv.), K₃PO₄ (3.56 g, 16.8 mmol, 3 equiv.), and 1,4-dioxane/H₂O (30 mL, 4:1) under argon atmosphere was stirred 2 hr at 90° C. The reaction mixture was concentrated to dryness under reduced pressure to give the crude product, which was purified by FCC (eluent: petroleum ether:ethyl acetate)=100/0 to 0/100) to yield the title compound (1.1 g, 49%) as a yellow oil.

Step D. N-(2,6-dimethylpyrimidin-4-yl)-5-[4-[[(2R)-1-methylazetidin-2-yl]methoxy]-2-(trideuteriomethyl)pyrazol-3-yl]pyrazolo[1,5-a]pyridin-2-amine. The title compound (70 mg, 50%) was prepared as described in Example 2, except tert-butyl (R)-2-(((5-(2-aminopyrazolo[1,5-a]pyridin-5-yl)-1-(methyl-d3)-1H-pyrazol-4-yl)oxy)methyl)azetidine-1-carboxylate (1 equiv.) was used instead of Intermediate 6 in Step A. MS (ESI): mass calcd. for C₂₂H₂₃D₃N₈O, 421.2. m/z found, 422.2 [M+H]⁺. ¹H NMR (400 MHz, CDCl₃) δ 8.39 (d, J=7.2 Hz, 1H), 7.52 (s, 1H), 7.47 (d, J=1.2 Hz, 1H), 7.35 (s, 1H), 7.06 (s, 1H), 6.86 (dd, J=2.0, 7.2 Hz, 1H), 6.58 (s, 1H), 4.01-3.96 (m, 2H), 3.48-3.41 (m, 1H), 3.40-3.29 (m, 1H), 2.89-2.80 (m, 1H), 2.61 (s, 3H), 2.45 (s, 3H), 2.34 (s, 3H), 2.06-1.95 (m, 2H)

### Example 132: N-(2-hydroxy-2-methyl-propyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide

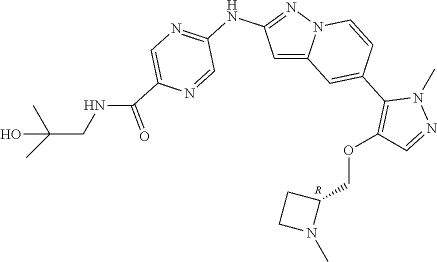

The title compound (80 mg, 5 steps, 15%) was prepared as described in Example 36, except methyl 5-chloropyrazine-2-carboxylate was used instead of methyl 6-bromonicotinate in Step A and 1-amino-2-methylpropan-2-ol was used instead of 3-amino-2,2-dimethylpropanenitrile in Step C. MS (ESI): mass calcd. for C₂₅H₃₁N₉O₃, 505.3. m/z found, 506.1 [M+H]⁺. ¹H NMR (400 MHz, DMSO-d6) δ 10.82 (s, 1H), 8.79 (s, 1H), 8.65 (d, J=7.2 Hz, 1H), 8.60 (s, 1H), 8.16 (t, J=6.0 Hz, 1H), 7.74 (s, 1H), 7.46 (s, 1H), 7.02 (s, 1H), 6.98 (dd, J=2.0, 7.2 Hz, 1H), 4.72 (s, 1H), 3.95-3.91 (m, 2H), 3.87 (s, 3H), 3.28 (d, J=6.4 Hz, 2H), 3.26-3.21 (m, 2H), 2.75-2.65 (m, 1H), 2.20 (s, 3H), 2.00-1.90 (m, 1H), 1.89-1.76 (m, 1H), 1.12 (s, 6H)

### Example 133: N-methyl-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide

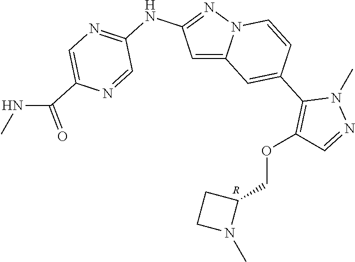

The title compound (70 mg, 5 steps, 20%) was prepared as described in Example 36, except methyl 5-chloropyrazine-2-carboxylate was used instead of methyl 6-bromonicotinate in Step A and MeNH2·HCl was used instead of 3-amino-2,2-dimethylpropanenitrile in Step C. MS (ESI): mass calcd. for C₂₂H₂₅N₉O₂, 447.2. m/z found, 448.2 [M+H]⁺. ¹H NMR (400 MHz, DMSO-d6) δ 10.77 (s, 1H), 8.76 (d, J=1.2 Hz, 1H), 8.66 (d, J=7.2 Hz, 1H), 8.58 (s, 1H), 8.51 (q, J=4.8 Hz, 1H), 7.73 (s, 1H), 7.46 (s, 1H), 7.01 (s, 1H), 6.97 (dd, J=2.0, 7.2 Hz, 1H), 3.96-3.92 (m, 2H), 3.87 (s, 3H), 3.29-3.19 (m, 2H), 2.81 (d, J=4.8 Hz, 3H), 2.75-2.68 (m, 1H), 2.20 (s, 3H), 1.98-1.90 (m, 1H), 1.89-1.80 (m, 1H)

### Example 134: N-(2-cyclopropyl-6-methyl-pyrimidin-4-yl)-5-[2-methyl-4-[[(2S)-1-methylazetidin-2-yl]methoxy]pyrazol-3-yl]pyrazolo[1,5-a]pyridin-2-amine

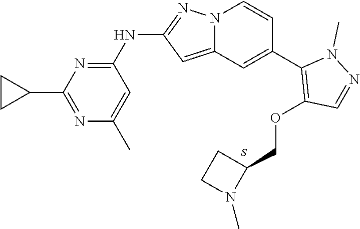

The title compound (79.5 mg, 3 steps, 48%) was prepared as described in Example 2, except 4-chloro-2-cyclopropyl-6-methylpyrimidine (1.2 equiv.) was used instead of 4-chloro-2,6-dimethylpyrimidine in Step A and the (S)-enantiomer of Intermediate 6 ((S)-tert-butyl 2-(((5-(2-aminopyrazolo[1,5-a]pyridin-5-yl)-1-methyl-1H-pyrazol-4-yl)oxy)methyl)azetidine-1-carboxylate) was used instead of the (R)-enantiomer in Step A. The material Brettphos-Pd-G3 (0.1 equiv.)/Brettphos (0.2 equiv.) was used instead of tBuBrettPhos in Step A. The material (S)-tert-butyl 2-(((5-(2-aminopyrazolo[1,5-a]pyridin-5-yl)-1-methyl-1H-pyrazol-4-yl)oxy)methyl)azetidine-1-carboxylate was prepared as described as Intermediate 6, except (S)-tert-butyl 2-((tosyloxy)methyl)azetidine-1-carboxylate (1 equiv.) was used instead of tert-butyl (R)-2-((tosyloxy)methyl)azetidine-1-carboxylate in Step A. To prepare (S)-tert-butyl 2-((tosyloxy)methyl)azetidine-1-carboxylate, a mixture consisting of 1-methyl-1H-pyrazol-4-ol (2.60 g, 26.5 mmol, 1 equiv.), (S)-tert-butyl 2-((tosyloxy)methyl)azetidine-1-carboxylate (9.05 g, 26.5 mmol, 1 equiv.), Cs₂CO₃ (25.9 g, 79.5 mmol, 3 equiv.) and DMF (30 mL) was stirred at 60° C. for 16 hr. The reaction mixture was filtered and purified by FCC (petroleum ether:ethyl acetate=1:0 to 0:1) to afford (S)-tert-butyl 2-((tosyloxy)methyl)azetidine-1-carboxylate (5.96 g, 84%) as a yellow oil. MS (ESI): mass calcd. for C₂₄H₂₈N₈O, 444.2. m/z found, 445.1 [M+H]⁺. ¹H NMR (400 MHz, DMSO-d6) δ 10.15 (s, 1H), 8.61 (d, J=7.2 Hz, 1H), 7.70 (d, J=1.2 Hz, 1H), 7.45 (s, 1H), 6.93 (dd, J=2.0, 7.2 Hz, 1H), 6.89-6.66 (m, 2H), 3.97-3.91 (m, 2H), 3.88 (s, 3H), 3.27-3.22 (m, 2H), 2.78-2.65 (m, 1H), 2.27 (s, 3H), 2.21 (s, 3H), 2.09-1.99 (m, 1H), 1.98-1.90 (m, 1H), 1.88-1.74 (m, 1H), 1.04-0.99 (m, 2H), 0.99-0.93 (m, 2H)

### Example 135: N-(2-cyanoethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide

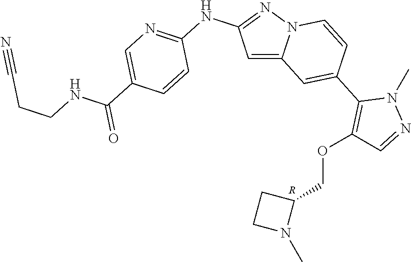

The title compound (37 mg, 13 steps, 13%) was prepared as described in Example 36, except 3-aminopropanenitrile (1.3 equiv.) was used instead of 3-amino-2,2-dimethylpropanenitrile in Step C. MS (ESI): mass calcd. for C₂₅H₂₇N₉O₂, 485.2. m/z found, 486.2 [M+H]⁺. ¹H NMR (400 MHz, CDCl₃) δ 8.76 (d, J=2.4 Hz, 1H), 8.40 (d, J=7.2 Hz, 1H), 8.05 (dd, J=2.4, 8.8 Hz, 1H), 8.01-7.93 (m, 1H), 7.50-7.45 (m, 2H), 7.35 (s, 1H), 6.85 (dd, J=2.0, 7.2 Hz, 1H), 6.74-6.66 (m, 1H), 6.60 (s, 1H), 4.05-3.94 (m, 2H), 3.91 (s, 3H), 3.78-3.70 (m, 2H), 3.50-3.42 (m, 1H), 3.41-3.29 (m, 1H), 2.91-2.81 (m, 1H), 2.78 (t, J=6.4 Hz, 2H), 2.35 (s, 3H), 2.11-1.95 (m, 2H)

### Example 136: N-(4-methyl-6,7-dihydro-5H-cyclopenta[b]pyridin-2-yl)-5-[2-methyl-4-[[(2R)-1-methylazetidin-2-yl]methoxy]pyrazol-3-yl]pyrazolo[1,5-a]pyridin-2-amine

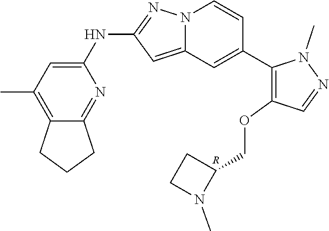

The title compound (240 mg, 3 steps, 28%) was prepared as described in Example 2, except 2-chloro-4-methyl-6,7-dihydro-5H-cyclopenta[b]pyridine was used instead of 4-chloro-2,6-dimethylpyrimidine in Step A and Brettphos-Pd-G3/Brettphos was used instead of t-BuBrettphos in Step A. MS (ESI): mass calcd. for C₂₅H₂₉N₇O, 443.2. m/z found, 444.1 [M+H]⁺. ¹H NMR (400 MHz, DMSO-d6) δ 9.53 (s, 1H), 8.54 (d, J=7.2 Hz, 67 (m, 1H), 2.19 (s, 3H), 2.18 (s, 3H), 2.07-1.99 (m, 2H), 1.97-1.89 (m, 1H), 1.88-1.77 (m, 1 Hz, 1H), 7.61 (d, J=1.2 Hz, 1H), 7.44 (s, 1H), 6.93 (s, 1H), 6.87-6.80 (m, 2H), 3.95-3.90 (m, 2H), 3.86 (s, 3H), 3.27-3.19 (m, 2H), 2.87-2.82 (m, 2H), 2.78-2.73 (m, 2H), 2.71-2.

### Example 137: N-(6-ethyl-2-methyl-pyrimidin-4-yl)-5-[2-methyl-4-[[(2S)-1-methylazetidin-2-yl]methoxy]pyrazol-3-yl]pyrazolo[1,5-a]pyridin-2-amine

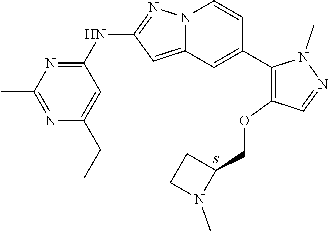

The title compound (550 mg, 3 steps, 12%) was prepared as described in Example 2, except 4-chloro-6-ethyl-2-methylpyrimidine was used instead of 4-chloro-2,6-dimethylpyrimidine in Step A and the S stereoisomer of Intermediate 6 ((S)-tert-butyl 2-(((5-(2-aminopyrazolo[1,5-a]pyridin-5-yl)-1-methyl-1H-pyrazol-4-yl)oxy)methyl)azetidine-1-carboxylate) was used instead of the R stereoisomer in Step A. The material Brettphos-Pd-G3/Brettphos was used instead of tBuBrettPhos in Step A. MS (ESI): mass calcd. for C₂₃H₂₈N₈O, 432.2. m/z found, 433.2 [M+H]⁺. ¹H NMR (400 MHz, DMSO-d6) δ 10.22 (s, 1H), 8.61 (d, J=7.2 Hz, 1H), 7.69 (d, J=0.8 Hz, 1H), 7.45 (s, 1H), 7.00 (br. s., 1H), 6.94 (dd, J=2.0, 7.2 Hz, 1H), 6.90 (br. s., 1H), 3.97-3.89 (m, 2H), 3.87 (s, 3H), 3.29-3.19 (m, 2H), 2.75-2.66 (m, 1H), 2.58 (q, J=7.2 Hz, 2H), 2.48 (s, 3H), 2.19 (s, 3H), 1.98-1.90 (m, 1H), 1.89-1.78 (m, 1H), 1.20 (t, J=8.0 Hz, 3H)

### Example 138: N-(5,6-dimethylpyrazin-2-yl)-5-[2-methyl-4-[[(2S)-1-methylazetidin-2-yl]methoxy]pyrazol-3-yl]pyrazolo[1,5-a]pyridin-2-amine

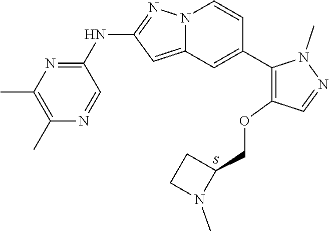

Step A. Tert-butyl (S)-2-((tosyloxy)methyl)azetidine-1-carboxylate. A solution consisting of (S)-1-Boc-2-azetidinemethanol (6.00 g, 32.0 mmol, 1 quiv.), DMAP (5.87 g, 48.1 mmol, 1.5 equiv.), and DCM (60 mL) was treated with TsCl (9.16 g, 48.1 mmol, 1.5 equiv.). The resultant mixture was stirred overnight at room-temperature. The reaction mixture was diluted with ethyl acetate (60 mL) and washed with 1N HCl (100 mL), saturated aqueous NaHCO₃ (100 mL), and brine (100 mL) in sequence.

The organic extract was dried over anhydrous Na₂SO₄, filtered, and evaporated to dryness under reduced pressure to give the crude product (12.2 g). The obtained crude product was purified by FCC (eluent: petroleum ether:ethyl acetate=100:0 to 70:30) to afford the title compound as a colorless oil (9.4 g, 86% yield).

Step B. Tert-butyl (S)-2-(((1-methyl-1H-pyrazol-4-yl)oxy)methyl)azetidine-1-carboxylate. A mixture consisting of 1-methyl-1H-pyrazol-4-ol (2.60 g, 26.5 mmol, 1 equiv.), (S)-tert-butyl 2-((tosyloxy)methyl)azetidine-1-carboxylate (9.05 g, 26.5 mmol, 1 equiv.), Cs₂CO₃ (25.9 g, 79.5 mmol, 3 equiv.) and DMF (30 mL) was stirred at 60° C. for 16 h. The reaction mixture was filtered and purified by FCC (petroleum ether:ethyl acetate=1:0 to 0:1) to afford the title compound (5.96 g, 84%) as a yellow oil.

Step C. Tert-butyl (S)-2-(((5-bromo-1-methyl-1H-pyrazol-4-yl)oxy)methyl)azetidine-1-carboxylate. A mixture consisting of (S)-tert-butyl 2-(((1-methyl-1H-pyrazol-4-yl)oxy)methyl)azetidine-1-carboxylate (5.96 g, 22.3 mmol, 1 equiv.), NBS (3.18 g, 17.8 mmol, 0.8 equiv.) and MeCN (60 mL) was stirred at room temperature for 1 hr. The reaction mixture was poured into water (300 mL) and concentrated under reduced pressure to remove solvent then extracted with EtOAc (300 mL×3). The combined organic extracts were washed with brine (100 mL×3), dried over anhydrous Na₂SO₄, filtered, and concentrated to dryness under reduced pressure to give the crude product, which was purified by FCC (eluent: petroleum ether:ethyl acetate=1:0 to 1:1) to afford the title compound (5.1 g, 66%) as a yellow oil.

Step D. (S)-tert-butyl 2-(((5-(2-aminopyrazolo[1,5-a]pyridin-5-yl)-1-methyl-1H-pyrazol-4-yl)oxy)methyl)azetidine-1-carboxylate. A mixture consisting of 5-(4,4,5,5-tetramethyl-1,3,2-dioxaborolan-2-yl)pyrazolo[1,5-a]pyridin-2-amine (4.20 g, 16.2 mmol, 1 equiv.), (S)-tert-butyl 2-(((5-bromo-1-methyl-1H-pyrazol-4-yl)oxy)methyl)azetidine-1-carboxylate (5.1 g, 15 mmol, 1.1 equiv.), Pd(dppf)Cl₂ (1.08 g, 1.47 mmol, 0.1 equiv.), potassium phosphate (7.82 g, 36.8 mmol, 2.5 equiv.), 1,4-dioxane/H₂O (50 mL, 4:1) under N2 was stirred at 90° C. for 4 hr. The mixture was concentrated under reduced pressure to give a crude product, which was purified by FCC (eluent: petroleum ether:ethyl acetate=1:0 to 0:1) to afford the title compound (5.2 g, 81%) as a yellow oil.

Step E. Tert-butyl (S)-2-(((5-(2-((6-ethyl-2-methylpyrimidin-4-yl)amino)pyrazolo[1,5-a]pyridin-5-yl)-1-methyl-1H-pyrazol-4-yl)oxy)methyl)azetidine-1-carboxylate. To a mixture consisting of (S)-tert-butyl 2-(((5-(2-aminopyrazolo[1,5-a]pyridin-5-yl)-1-methyl-1H-pyrazol-4-yl)oxy)methyl)azetidine-1-carboxylate (500 mg, 1.26 mmol, 1 equiv.), 4-chloro-6-ethyl-2-methylpyrimidine (216 mg, 1.38 mmol, 1.1 equiv.), Brettphos (135 mg, 0.251 mmol 0.1 equiv.) and Cs₂CO₃ (1.23 g, 3.77 mmol, 3 equiv.) in 1,4-dioxane (15 mL) and was added Brettphos-Pd-G3 (135 mg, 0.251 mmol, 0.2 equiv.) under N2 atmosphere. The resulting mixture was stirred at 50° C. for 3 hr. The reaction mixture was concentrated under reduced pressure to give the crude product, which was purified by FCC (eluent: MeOH:ethyl acetate=0:1 to 1:1) to afford the title compound (550 mg, 85%) as a yellow solid.

Step F. (S)-5-(4-(azetidin-2-ylmethoxy)-1-methyl-1H-pyrazol-5-yl)-N-(6-ethyl-2-methylpyrimidin-4-yl)pyrazolo[1,5-a]pyridin-2-amine. A mixture consisting of (S)-tert-butyl 2-(((5-(2-((6-ethyl-2-methylpyrimidin-4-yl)amino)pyrazolo[1,5-a]pyridin-5-yl)-1-methyl-1H-pyrazol-4-yl)oxy)methyl)azetidine-1-carboxylate (525 mg, 1.01 mmol, 1 equiv.) and TFA/DCM (20 mL, 1:5) was stirred at room temperature for 2 hr. The reaction mixture was concentrated to dryness under reduced pressure to give the title compound, which was basified with aqueous NaHCO₃ to PH=8. The mixture was frozen using dry ice/ethanol, and then lyophilized to dryness to afford the title compound (380 mg, 78%) as a yellow solid.

Step G. N-(5,6-dimethylpyrazin-2-yl)-5-[2-methyl-4-[[(2S)-1-methylazetidin-2-yl]methoxy]pyrazol-3-yl]pyrazolo[1,5-a]pyridin-2-amine. A mixture consisting of (S)-5-(4-(azetidin-2-ylmethoxy)-1-methyl-1H-pyrazol-5-yl)-N-(6-ethyl-2-methylpyrimidin-4-yl)pyrazolo[1,5-a]pyridin-2-amine (170 mg, 0.355 mmol, 1 equiv.), H(CHO)n (32.0 mg, 0.355 mmol, 1 equiv.), NaBH₃CN (66.9 mg, 1.07 mmol, 3 equiv.) and MeOH (10 mL) was stirred at room temperature for 2 hr. The reaction mixture was filtered and purified by preparative HPLC using a Phenomenex Gemini-NX C18 75×30 mm×3 μm (eluent: 27% to 57% (v/v) water (0.05% NH₃H₂O+10 mM NH₄HCO₃)-ACN to afford pure product. The product was suspended in water (10 mL), the mixture frozen using dry ice/ethanol, and then lyophilized to dryness to afford the title compound (35.1 mg, 21%) as a white solid. MS (ESI): mass calcd. for C₂₂H₂₆N₈O, 418.2. m/z found, 419.2 [M+H]⁺. ¹H NMR (400 MHz, DMSO-d6) δ 9.94 (s, 1H), 8.59 (d, J=7.2 Hz, 1H), 8.44 (s, 1H), 7.65 (s, 1H), 7.45 (s, 1H), 6.90 (dd, J=1.6, 7.2 Hz, 1H), 6.84 (s, 1H), 3.97-3.89 (m, 2H), 3.87 (s, 3H), 3.28-3.20 (m, 2H), 2.76-2.67 (m, 1H), 2.43 (s, 3H), 2.38 (s, 3H), 2.20 (s, 3H), 1.98-1.89 (m, 1H), 1.89-1.79 (m, 1H)

### Example 139: N-(2,6-dimethylpyrimidin-4-yl)-5-[2-methyl-4-[[(2S)-1-methylazetidin-2-yl]methoxy]pyrazol-3-yl]pyrazolo[1,5-a]pyridin-2-amine

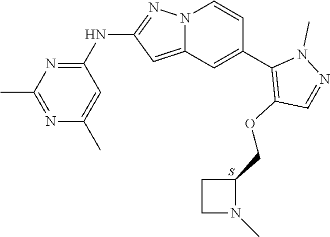

Step A. Tert-butyl (S)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. To a mixture consisting of Intermediate 10 (320 mg, 0.954 mmol, 1 equiv.), (S)-tert-butyl 2-((tosyloxy)methyl)azetidine-1-carboxylate (326 mg, 0.954 mmol, 1 equiv.) and DMF (5 mL) was added Cs₂CO₃ (933 mg, 2.86 mmol, 3 equiv.) and heated to 80° C. via microwave irradiation for 1 hour before cooling to room-temperature. The reaction mixture was concentrated under vacuum to give a residue, which was purified by FCC (eluent: PE:(EA:EtOH=3:1)=0% to 40%) afford the title product (140 mg, 29%) as a yellow oil.

Step B. N-(2,6-dimethylpyrimidin-4-yl)-5-[2-methyl-4-[[(2S)-1-methylazetidin-2-yl]methoxy]pyrazol-3-yl]pyrazolo[1,5-a]pyridin-2-amine. The title compound (67.2 mg, 2 steps, 84%) was prepared as described in Steps F-G of Example 138. MS (ESI): mass calcd. for C₂₂H₂₆N₈O, 418.2. m/z found, 419.1 [M+H]⁺. ¹H NMR (400 MHz, CDCl₃) δ 8.40 (d, J=7.2 Hz, 1H), 7.62 (s, 1H), 7.47 (s, 1H), 7.35 (s, 1H), 7.06 (s, 1H), 6.86 (dd, J=2.0, 7.2 Hz, 1H), 6.59 (s, 1H), 4.01-3.96 (m, 2H), 3.91 (s, 3H), 3.47-3.39 (m, 1H), 3.39-3.30 (m, 1H), 2.88-2.79 (m, 1H), 2.60 (s, 3H), 2.45 (s, 3H), 2.33 (s, 3H), 2.05-1.93 (m, 2H)

### Example 140: 5-[2-(difluoromethyl)-4-[[(2R)-1-methylazetidin-2-yl]methoxy]pyrazol-3-yl]-N-(2,6-dimethylpyrimidin-4-yl)pyrazolo[1,5-a]pyridin-2-amine

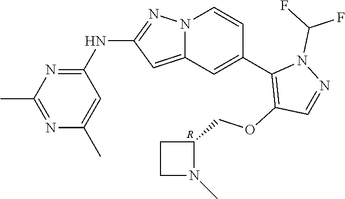

The title compound (39.7 mg, 3 steps, 26%) was prepared as described in Intermediate 12, except N-(2,6-dimethylpyrimidin-4-yl)-5-(4,4,5-trimethyl-1,3,2-dioxaborolan-2-yl)pyrazolo[1,5-a]pyridin-2-amine (Intermediate 11, 1.05 equiv.) was used instead of Intermediate 5 in Step D to give tert-butyl (R)-2-(((1-(difluoromethyl)-5-(2-((2,6-dimethylpyrimidin-4-yl)amino)pyrazolo[1,5-a]pyridin-5-yl)-1H-pyrazol-4-yl)oxy)methyl)azetidine-1-carboxylate. The deprotection and subsequent methylation was followed as described in Example 33, Step B. MS (ESI): mass calcd. for C₂₂H₂₄F₂N₈O, 454.2. m/z found, 455.2 [M+H]⁺. ¹H NMR (500 MHz, DMSO-d6) δ 10.23 (s, 1H), 8.67-8.62 (m, 1H), 7.95 (s, 1H), 7.86-7.60 (m, 2H), 6.99 (s, 1H), 6.92 (s, 1H), 6.83 (dd, J=7.2, 2.0 Hz, 1H), 4.07-3.97 (m, 2H), 3.25-3.19 (m, 2H), 2.72-2.65 (m, 1H), 2.46 (s, 3H), 2.30 (s, 3H), 2.16 (s, 3H), 1.96-1.88 (m, 1H), 1.88-1.79 (m, 1H).

### Example 141: 5-[2-methyl-4-[[(2R)-1-methylazetidin-2-yl]methoxy]pyrazol-3-yl]-N-(5-methylpyridazin-3-yl)pyrazolo[1,5-a]pyridin-2-amine

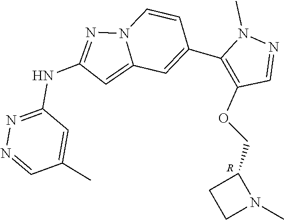

The title compound (19 mg, 3 steps, 7%) was prepared as described in Example 138 (Steps E-G), except 3-chloro-5-methylpyridazine (2 equiv.) was used instead of 4-chloro-6-ethyl-2-methylpyrimidine in Step E and Intermediate 6 was used instead of (S)-tert-butyl 2-(((5-(2-aminopyrazolo[1,5-a]pyridin-5-yl)-1-methyl-1H-pyrazol-4-yl)oxy)methyl)azetidine-1-carboxylate in Step E. MS (ESI): mass calcd. for C₂₁H₂₄N₈O, 404.2. m/z found, 405.2 [M+H]⁺. ¹H NMR (500 MHz, Chloroform-d) δ 9.00 (s, 1H), 8.65 (d, J=1.8 Hz, 1H), 8.41 (dt, J=7.2, 1.0 Hz, 1H), 7.91 (s, 1H), 7.48 (dd, J=1.9, 0.9 Hz, 1H), 7.36 (s, 1H), 6.84 (dd, J=7.2, 1.9 Hz, 1H), 6.66 (s, 1H), 4.06-3.96 (m, 2H), 3.93 (s, 3H), 3.47-3.39 (m, 1H), 3.37-3.31 (m, 1H), 2.87-2.79 (m, 1H), 2.40 (s, 3H), 2.35 (s, 3H), 2.08-1.99 (m, 1H), 2.03-1.93 (m, 1H).

### Example 142: 5-[2-methyl-4-[[(2R)-1-methylazetidin-2-yl]methoxy]pyrazol-3-yl]-N-(1-methylpyrazol-4-yl)pyrazolo[1,5-a]pyridin-2-amine

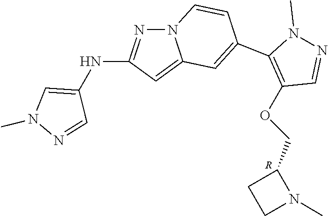

The title compound (140 mg, 3 steps, 14%) was prepared as described in Example 2, except 4-bromo-1-methyl-1h-pyrazole (1.2 equiv.) was used instead of 4-chloro-2,6-dimethylpyrimidine in Step A and tris(dibenzylideneacetone)dipalladium-chloroform adduct (0.1 equiv.)/di-tert-butyl(2′,4′,6′-triisopropyl-3,6-dimethoxy-[1,1′-biphenyl]-2-yl)phosphine was used instead of t-BuBrettPhos in Step A. MS (ESI): mass calcd. for C₂₀H₂₄N₈O, 392.2. m/z found, 393.2 [M+H]⁺. ¹H NMR (500 MHz, Chloroform-d) δ 8.25 (dt, J=7.1, 0.9 Hz, 1H), 7.59 (d, J=0.8 Hz, 1H), 7.45 (d, J=0.8 Hz, 1H), 7.34-7.24 (m, 2H), 6.67 (dd, J=7.1, 1.9 Hz, 1H), 6.21 (s, 1H), 5.85 (d, J=0.8 Hz, 1H), 3.99-3.91 (m, 2H), 3.88 (d, J=7.1 Hz, 5H), 3.41-3.37 (m, 1H), 3.33-3.27 (m, 1H), 2.83-2.76 (m, 1H), 2.43 (s, 1H), 2.31 (s, 3H), 2.04-1.89 (m, 2H).

### Example 143: 2-methyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one

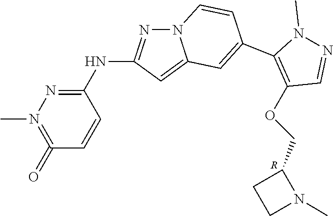

The title compound (450 mg, 3 steps, 48%) was prepared as described in Example 2, except 6-bromo-2-methyl-3(2h)-pyridazinone was used instead of 4-chloro-2,6-dimethylpyrimidine in Step A. MS (ESI): mass calcd. for C₂₁H₂₄N₈O₂, 420.2. m/z found, 421.2 [M+H]⁺. ¹H NMR (400 MHz, Chloroform-d) δ 8.29 (dt, J=7.2, 0.9 Hz, 1H), 8.11 (s, 1H), 7.47 (dd, J=2.0, 0.9 Hz, 1H), 7.37-7.26 (m, 2H), 6.96 (d, J=9.7 Hz, 1H), 6.84 (dd, J=7.2, 1.9 Hz, 1H), 6.72 (d, J=0.8 Hz, 1H), 3.98 (d, J=5.5 Hz, 2H), 3.91 (s, 3H), 3.78 (s, 3H), 3.47-3.37 (m, 1H), 3.36-3.28 (m, 1H), 2.85-2.78 (m, 1H), 2.32 (s, 3H), 2.07-1.91 (m, 2H).

### Example 144: N-(2-cyclopropyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine

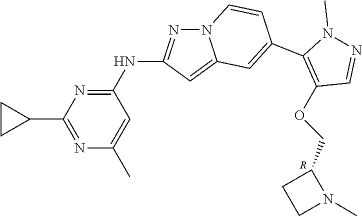

The title compound (269 mg, 3 steps, 72%) was prepared as described in Example 2, except 4-chloro-2-cyclopropyl-6-methylpyrimidine was used instead of 4-chloro-2,6-dimethylpyrimidine in Step A. MS (ESI): mass calcd. for C₂₄H₂₈N₈O, 444.2. m/z found, 445.2 [M+H]⁺. ¹H NMR (500 MHz, Chloroform-d) δ 8.40-8.33 (m, 2H), 7.48 (dd, J=1.9, 0.9 Hz, 1H), 7.34 (s, 1H), 6.83 (dd, J=7.2, 1.9 Hz, 1H), 6.78 (s, 1H), 6.68 (s, 1H), 4.02-3.93 (m, 2H), 3.91 (s, 3H), 3.46 (s, 1H), 3.43-3.38 (m, 1H), 3.35-3.28 (m, 1H), 2.84-2.77 (m, 1H), 2.38 (s, 2H), 2.32 (s, 3H), 2.12 (tt, J=8.1, 4.7 Hz, 1H), 2.04-1.91 (m, 2H), 1.15 (dt, J=4.7, 3.1 Hz, 2H), 1.05-0.94 (m, 2H).

### Example 145: N-(6-ethyl-2-methyl-pyrimidin-4-yl)-5-[2-methyl-4-[[(2R)-1-methylazetidin-2-yl]methoxy]pyrazol-3-yl]pyrazolo[1,5-a]pyridin-2-amine

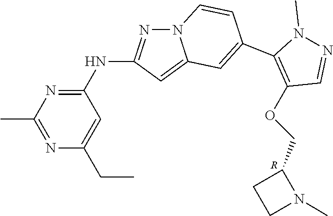

The title compound (271 mg, 3 steps, 62%) was prepared as described in Example 2, except 4-chloro-6-ethyl-2-methylpyrimidine was used instead of 4-chloro-2,6-dimethylpyrimidine in Step A. MS (ESI): mass calcd. for C₂₃H₂₈N₈O, 432.2. m/z found, 433.3 [M+H]⁺. ¹H NMR (500 MHz, Chloroform-d) δ 8.51-8.45 (m, 2H), 7.47 (dd, J=1.9, 0.9 Hz, 1H), 7.35 (s, 1H), 6.99 (s, 1H), 6.86 (dd, J=7.2, 1.9 Hz, 1H), 6.63 (s, 1H), 4.02-3.94 (m, 2H), 3.91 (s, 3H), 3.49-3.37 (m, 1H), 3.35-3.29 (m, 1H), 2.84-2.78 (m, 1H), 2.70 (q, J=7.6 Hz, 2H), 2.61 (s, 3H), 2.32 (s, 3H), 2.05-1.91 (m, 2H), 1.29 (t, J=7.6 Hz, 3H).

### Example 146: N-(2-methoxy-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine

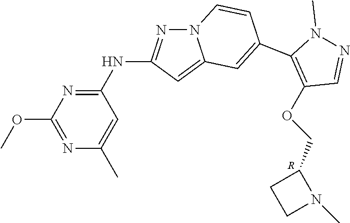

The title compound (283 mg, 3 steps, 61%) was prepared as described in Example 2, except 4-chloro-2-methoxy-6-methylpyrimidine was used instead of 4-chloro-2,6-dimethylpyrimidine in Step A. MS (ESI): mass calcd. for C₂₂H₂₆N₈O₂, 434.2. m/z found, 435.2 [M+H]⁺. ¹H NMR (500 MHz, Chloroform-d) δ 9.31 (d, J=11.1 Hz, 1H), 8.36 (dd, J=7.4, 2.1 Hz, 1H), 7.40 (d, J=2.0 Hz, 1H), 7.27 (d, J=2.5 Hz, 1H), 6.80-6.71 (m, 2H), 6.61 (s, 1H), 3.95-3.86 (m, 4H), 3.83 (d, J=2.7 Hz, 3H), 3.39-3.30 (m, 2H), 3.25 (dd, J=9.8, 4.7 Hz, 1H), 2.72 (td, J=8.8, 4.7 Hz, 1H), 2.26 (dd, J=10.7, 3.0 Hz, 6H), 1.95-1.87 (m, 2H).

### Example 147: N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine

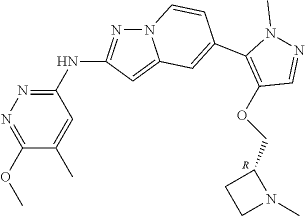

The title compound (47 mg, 3 steps, 15%) was prepared as described in Example 2, except 6-chloro-3-methoxy-4-methylpyridazine was used instead of 4-chloro-2,6-dimethylpyrimidine in Step A. MS (ESI): mass calcd. for C₂₂H₂₆N₈O₂, 434.2. m/z found, 435.2 [M+H]⁺. ¹H NMR (400 MHz, Chloroform-d) δ 8.89 (s, 1H), 8.42 (dt, J=7.1, 1.0 Hz, 1H), 7.67 (s, 1H), 7.44 (dd, J=1.9, 0.9 Hz, 1H), 7.35 (s, 1H), 6.84-6.75 (m, 2H), 4.13 (s, 3H), 4.04-3.93 (m, 2H), 3.92 (s, 3H), 3.49 (s, OH), 3.42 (td, J=7.2, 2.9 Hz, 1H), 3.36-3.28 (m, 1H), 2.86-2.76 (m, 1H), 2.33 (s, 3H), 2.27 (d, J=1.1 Hz, 3H), 2.08-1.91 (m, 2H).

### Example 148: 2-methyl-N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine

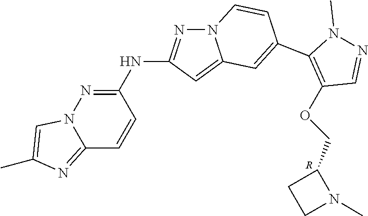

The title compound (138 mg, 3 steps, 45%) was prepared as described in Example 2, except 6-chloro-2-methylimidazo[1,2-b]pyridazine was used instead of 4-chloro-2,6-dimethylpyrimidine in Step A. MS (ESI): mass calcd. for C₂₃H₂₅N₉O, 443.2. m/z found, 444.2 [M+H]⁺. ¹H NMR (500 MHz, Chloroform-d) δ 8.68-8.63 (m, 1H), 8.33 (dt, J=7.1, 0.9 Hz, 1H), 7.69-7.62 (m, 1H), 7.60-7.56 (m, 1H), 7.50 (dd, J=2.0, 0.9 Hz, 1H), 7.35 (s, 1H), 6.98 (d, J=0.8 Hz, 1H), 6.88-6.77 (m, 2H), 3.99 (d, J=5.6 Hz, 2H), 3.92 (s, 3H), 3.49-3.38 (m, 2H), 3.36-3.29 (m, 1H), 2.85-2.74 (m, 2H), 2.46 (d, J=0.8 Hz, 3H), 2.06-1.93 (m, 3H).

### Example 149: N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine

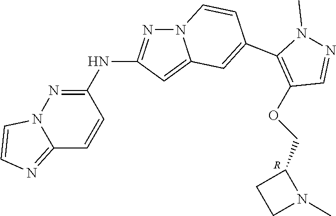

The title compound (317 mg, 3 steps, 90%) was prepared as described in Example 2, except 6-chloroimidazo[1,2-b]pyridazine was used instead of 4-chloro-2,6-dimethylpyrimidine in Step A. MS (ESI): mass calcd. for C₂₂H₂₃N₉O, 429.2. m/z found, 430.2 [M+H]⁺. ¹H NMR (400 MHz, Chloroform-d) δ 8.68 (s, 1H), 8.34 (dt, J=7.2, 0.9 Hz, 1H), 7.84-7.75 (m, 2H), 7.63 (d, J=1.1 Hz, 1H), 7.52 (dd, J=1.9, 0.9 Hz, 1H), 7.36 (s, 1H), 7.03 (d, J=0.8 Hz, 1H), 6.92-6.83 (m, 2H), 4.03-3.95 (m, 2H), 3.92 (s, 3H), 3.51-3.37 (m, 2H), 3.39-3.30 (m, 1H), 2.34 (s, 3H), 2.08-1.92 (m, 2H).

### Example 150: 5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine

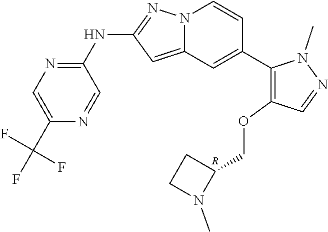

The title compound (204 mg, 3 steps, 28%) was prepared as described in Example 2, except 2-chloro-5-(trifluoromethyl)pyrazine was used instead of 4-chloro-2,6-dimethylpyrimidine in Step A. MS (ESI): mass calcd. for C₂₁H₂₁F₃N₈O, 458.2. m/z found, 459.2 [M+H]⁺. ¹H NMR (500 MHz, Chloroform-d) δ 8.75 (d, J=1.4 Hz, 1H), 8.56 (dd, J=1.4, 0.7 Hz, 1H), 8.38 (dt, J=7.2, 0.9 Hz, 1H), 8.31 (s, 1H), 7.52 (dd, J=1.9, 0.9 Hz, 1H), 7.37 (s, 1H), 6.91 (dd, J=7.2, 1.9 Hz, 1H), 6.79 (d, J=0.9 Hz, 1H), 4.04-3.98 (m, 2H), 3.94 (s, 3H), 3.49-3.42 (m, 1H), 3.39-3.32 (m, 1H), 2.89-2.80 (m, 1H), 2.36 (s, 3H), 2.09-1.96 (m, 2H).

### Example 151: N-(2-tert-butyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine

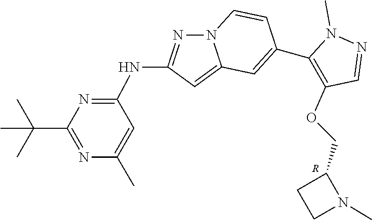

The title compound (397 mg, 3 steps, 89%) was prepared as described in Example 2, except 2-tert-butyl-4-chloro-6-methylpyrimidine was used instead of 4-chloro-2,6-dimethylpyrimidine in Step A. MS (ESI): mass calcd. for C₂₅H₃₂N₈O, 460.3. m/z found, 461.3 [M+H]⁺. ¹H NMR (500 MHz, Chloroform-d) δ 8.36 (dt, J=7.1, 0.9 Hz, 1H), 7.88 (s, 1H), 7.51 (dd, J=1.9, 0.9 Hz, 1H), 7.35 (s, 1H), 6.88-6.80 (m, 2H), 6.75 (s, 1H), 4.03-3.94 (m, 2H), 3.93 (s, 3H), 3.50-3.38 (m, 2H), 3.38-3.29 (m, 1H), 2.84-2.79 (m, 1H), 2.42 (s, 2H), 2.34 (s, 3H), 2.06-1.92 (m, 2H), 1.43 (s, 9H).

### Example 152: 2,4-dimethyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one

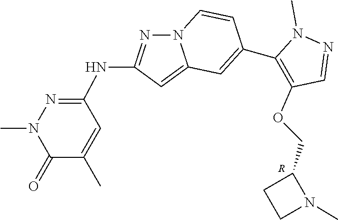

The title compound (296 mg, 3 steps, 62%) was prepared as described in Example 2, except 6-chloro-2,4-dimethylpyridazin-3(2h)-one was used instead of 4-chloro-2,6-dimethylpyrimidine in Step A. MS (ESI): mass calcd. for C₂₂H₂₆N₈O₂, 434.2. m/z found, 435.2 [M+H]⁺. ¹H NMR (500 MHz, Chloroform-d) δ 8.29 (dt, J=7.2, 0.9 Hz, 1H), 7.80-7.75 (m, 1H), 7.46 (dd, J=2.0, 1.0 Hz, 1H), 7.35 (s, 1H), 7.10 (q, J=1.3 Hz, 1H), 6.83 (dd, J=7.2, 1.9 Hz, 1H), 6.72 (d, J=0.8 Hz, 1H), 4.00-3.94 (m, 2H), 3.91 (s, 2H), 3.78 (s, 3H), 3.47 (s, 1H), 3.42 (td, J=7.2, 2.8 Hz, 1H), 3.37-3.29 (m, 1H), 2.84-2.78 (m, 1H), 2.32 (s, 3H), 2.22 (d, J=1.3 Hz, 3H), 2.05-1.92 (m, 2H).

### Example 153: N-[2-(cyclopropylmethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine

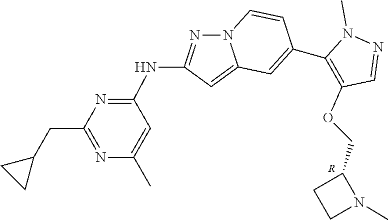

The title compound (326 mg, 3 steps, 76%) was prepared as described in Example 2, except 4-chloro-2-(cyclopropylmethyl)-6-methylpyrimidine was used instead of 4-chloro-2,6-dimethylpyrimidine in Step A. MS (ESI): mass calcd. for C₂₅H₃₀N₈O, 458.3. m/z found, 459.2 [M+H]⁺. ¹H NMR (500 MHz, Chloroform-d) δ 8.30 (dt, J=7.2, 0.9 Hz, 1H), 8.09 (s, 1H), 7.34 (dd, J=2.0, 0.9 Hz, 1H), 7.20 (s, 1H), 6.80 (s, 1H), 6.70 (dd, J=7.2, 1.9 Hz, 1H), 6.57 (s, 1H), 3.88-3.80 (m, 2H), 3.77 (s, 3H), 3.34-3.23 (m, 1H), 3.21-3.14 (m, 1H), 2.69-2.63 (m, 1H), 2.59 (d, J=7.1 Hz, 2H), 2.29 (s, 2H), 2.18 (s, 3H), 1.91-1.77 (m, 2H), 1.20-1.09 (m, 1H), 0.42-0.33 (m, 2H), 0.16 (dt, J=6.0, 4.5 Hz, 2H).

### Example 154: N-(5-methoxypyrazin-2-yl)-5-[2-methyl-4-[[(2R)-1-methylazetidin-2-yl]methoxy]pyrazol-3-yl]pyrazolo[1,5-a]pyridin-2-amine

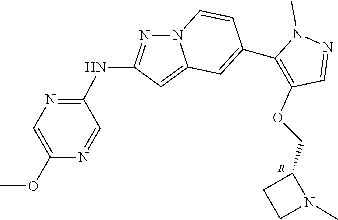

The title compound (163 mg, 3 steps, 43%) was prepared as described in Example 2, except 2-chloro-5-methoxypyrazine was used instead of 4-chloro-2,6-dimethylpyrimidine in Step A. MS (ESI): mass calcd. for C₂₁H₂₄N₈O₂, 420.2. m/z found, 421.2 [M+H]⁺. ¹H NMR (400 MHz, Chloroform-d) δ 8.45 (d, J=1.5 Hz, 1H), 8.37 (dt, J=7.3, 0.9 Hz, 1H), 7.96 (d, J=1.5 Hz, 1H), 7.77 (s, 1H), 7.41 (dd, J=1.9, 0.9 Hz, 1H), 7.35 (s, 1H), 6.79 (dd, J=7.2, 1.9 Hz, 1H), 6.44 (d, J=0.8 Hz, 1H), 4.03-3.88 (m, 8H), 3.50-3.26 (m, 2H), 2.87-2.77 (m, 1H), 2.33 (s, 3H), 2.08-1.90 (m, 2H).

### Example 155: N-[5-(difluoromethoxy)pyrazin-2-yl]-5-[2-methyl-4-[[(2R)-1-methylazetidin-2-yl]methoxy]pyrazol-3-yl]pyrazolo[1,5-a]pyridin-2-amine

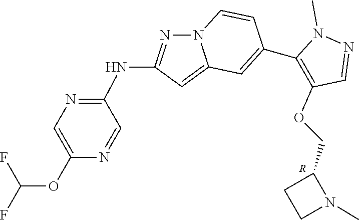

The title compound (182 mg, 3 steps, 48%) was prepared as described in Example 2, except 2-chloro-5-(difluoromethoxy)pyrazine was used instead of 4-chloro-2,6-dimethylpyrimidine in Step A. MS (ESI): mass calcd. for C₂₁H₂₂F₂N₈O₂, 456.2. m/z found, 457.2 [M+H]⁺. ¹H NMR (400 MHz, Chloroform-d) δ 8.52-8.42 (m, 2H), 8.37 (dt, J=7.2, 0.9 Hz, 1H), 8.05 (d, J=1.4 Hz, 1H), 7.47-7.40 (m, 1H), 7.35 (s, 1H), 6.83 (dd, J=7.2, 1.9 Hz, 1H), 6.51 (d, J=0.8 Hz, 1H), 4.02-3.93 (m, 2H), 3.91 (s, 3H), 3.49-3.38 (m, 1H), 3.38-3.30 (m, 1H), 2.87-2.76 (m, 1H), 2.64 (s, 1H), 2.33 (s, 3H), 2.07-1.94 (m, 2H).

### Example 156: (1*S, 2*S)—N-[5-[2-methyl-4-[((R)-1-methylazetidin-2-yl)methoxy]pyrazol-3-yl]pyrazolo[1,5-a]pyridin-2-yl]-2-phenyl-cyclopropanecarboxamide

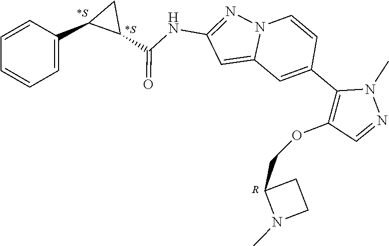

Step A. (1*S,2*S)-2-phenyl-N-(5-(4,4,5,5-tetramethyl-1,3,2-dioxaborolan-2-yl)pyrazolo[1,5-a]pyridin-2-yl)cyclopropane-1-carboxamide. The mixture of 5-(4,4,5,5-tetramethyl-1,3,2-dioxaborolan-2-yl)pyrazolo[1,5-a]pyridin-2-amine (Intermediate 5, 500 mg, 1.93 mmol, 1 equiv.), trans-2-phenyl-1-cyclopropanecarboxylic acid (313 mg, 1.93 mmol, 1 equiv.), HATU (733 mg, 1.93 mmol, 1 equiv.) and triethylamine (0.805 mL, 5.88 mmol, 3 equiv.) in DCM (10 mL) was stirred at room temperature for 8 hr. The reaction mixture was washed with saturated NaHCO₃ solution (5 mL) then sodium hydroxide (5 mL). The reaction mixture was separated, concentrated, then purified by FCC (10% acetone/DCM) providing the crude title product (610 mg, 78%).

Step B. (1*S, 2*S)—N-[5-[2-methyl-4-[((R)-1-methylazetidin-2-yl)methoxy]pyrazol-3-yl]pyrazolo[1,5-a]pyridin-2-yl]-2-phenyl-cyclopropanecarboxamide. A mixture consisting of (1S,2S)-2-phenyl-N-(5-(4,4,5,5-tetramethyl-1,3,2-dioxaborolan-2-yl)pyrazolo[1,5-a]pyridin-2-yl)cyclopropane-1-carboxamide (0.204 mg, 0.507 mmol, 1 equiv.), (R)-5-bromo-1-methyl-4-((1-methylazetidin-2-yl)methoxy)-1H-pyrazole (145 mg, 0.557 mmol, 1.1 equiv.), potassium fluoride (0.088 g, 1.52 mmol, 3 equiv.) water (0.778 mL, 43.1 mmol, 85 equiv.), Pd(dtbpf)Cl₂ (33 mg, 0.05 mmol, 0.1 equiv.) in 1,4-dioxane (3.67 mL, 43.1 mmol, 85 equiv.). The reaction was heated to 90° C. for 2 hr. The crude was diluted with brine and EtOAc. The aq. layer was separated. The organic layer was dried over MgSO₄, filtered, and concentrated under vacuo. The filtrated from the precipitation was reabsorbed onto silica gel and flashed with 30-70% EtOAc to 5% MeOH/DCM to provide a racemic mix of the title compound. The reaction was further purified via Chiralcel OD-H 30% ethanol (0.1% DEA)/CO₂, 100 bar. The first eluted was the title compound (57 mg, 25%). MS (ESI): mass calcd. for C₂₆H₂₈N₆O₂, 456.2. m/z found, 457.1 [M+H]⁺. ¹H NMR (500 MHz, Methanol-d4) δ 8.52-8.35 (m, 1H), 7.65 (dd, J=1.9, 0.9 Hz, 1H), 7.44 (s, 1H), 7.33-7.25 (m, 2H), 7.23-7.13 (m, 3H), 7.02-6.89 (m, 2H), 4.06-3.96 (m, 2H), 3.89 (s, 3H), 3.50-3.41 (m, 1H), 3.39-3.36 (m, 1H), 3.00-2.87 (m, 1H), 2.59-2.50 (m, 1H), 2.31 (s, 3H), 2.21-2.14 (m, 1H), 2.10-1.94 (m, 2H), 1.70-1.57 (m, 1H), 1.46-1.33 (m, 1H).

### Example 157: (1*R,2*R)—N-[5-[2-methyl-4-[((R)₁-methylazetidin-2-yl)methoxy]pyrazol-3-yl]pyrazolo[1,5-a]pyridin-2-yl]-2-phenyl-cyclopropanecarboxamide

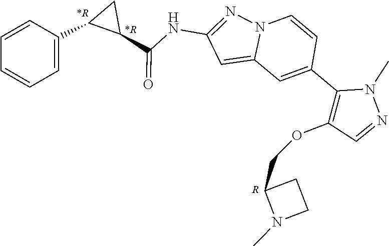

The title compound (49 mg, 2 steps, 17%) was prepared as described in Example 156, except the second eluted peak via Chiralcel OD-H 30% ethanol (0.1% DEA)/CO₂, 100 bar was the title compound. MS (ESI): mass calcd. for C₂₆H₂₈N₆O₂, 456.2. m/z found, 457.1 [M+H]⁺. ¹H NMR (500 MHz, Methanol-d4) δ 8.52-8.35 (m, 1H), 7.65 (dd, J=1.9, 0.9 Hz, 1H), 7.44 (s, 1H), 7.33-7.25 (m, 2H), 7.23-7.13 (m, 3H), 7.02-6.89 (m, 2H), 4.06-3.96 (m, 2H), 3.89 (s, 3H), 3.50-3.41 (m, 1H), 3.39-3.36 (m, 1H), 3.00-2.87 (m, 1H), 2.59-2.50 (m, 1H), 2.31 (s, 3H), 2.21-2.14 (m, 1H), 2.10-1.94 (m, 2H), 1.70-1.57 (m, 1H), 1.46-1.33 (m, 1H).

### Example 158: (1S,2R)—N-[5-[2-methyl-4-[[(2R)-1-methylazetidin-2-yl]methoxy]pyrazol-3-yl]pyrazolo[1,5-a]pyridin-2-yl]-2-phenyl-cyclopropanecarboxamide

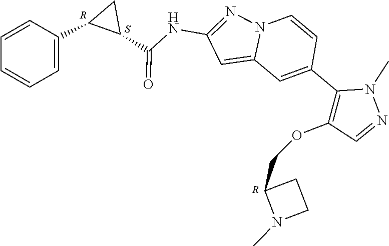

The title compound (45 mg, 2 steps, 44%) was prepared as described in Example 156, except cis-2-phenyl-1-cyclopropanecarboxylic acid was used instead of the trans-2-phenyl-1-cyclopropanecarboxylic acid. The compound was purified via silica gel and flashed with 30-70% EtOAc to 5% MeOH/DCM. MS (ESI): mass calcd. for C₂₆H₂₈N₆O₂, 456.2. m/z found, 457.1 [M+H]⁺. ¹H NMR (500 MHz, Methanol-d4) δ 8.30 (dd, J=7.2, 1.0 Hz, 1H), 7.48-7.39 (m, 1H), 7.36-7.26 (m, 1H), 7.23-7.16 (m, 2H), 7.13-7.06 (m, 2H), 7.06-6.95 (m, 1H), 6.82 (dd, J=7.2, 1.9 Hz, 1H), 6.54 (d, J=0.8 Hz, 1H), 3.93-3.86 (m, 2H), 3.75 (s, 3H), 3.39-3.32 (m, 1H), 3.28-3.23 (m, 1H), 2.82-2.75 (m, 1H), 2.59-2.49 (m, 1H), 2.28-2.22 (m, 1H), 2.18 (s, 3H), 2.03-1.84 (m, 2H), 1.71-1.63 (m, 1H), 1.34-1.22 (m, 1H).

### Example 159: (1*S,2*R)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide

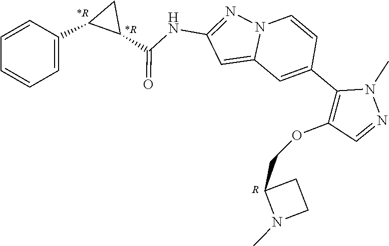

The title compound (108 mg, 2 steps, 44%) was prepared as described in Example 156, except Rac-cis-2-phenyl-1-cyclopropanecarboxylic acid was used instead of the trans-2-phenyl-1-cyclopropanecarboxylic acid. The racemic material was purified via prep. SFC via Chiralcel OD-H 30% ethanol (0.1% DEA)/CO₂, 100 bar where the second eluting peak was the title compound. MS (ESI): mass calcd. for C₂₆H₂₈N₆O₂, 456.2. m/z found, 457.2 [M+H]⁺. ¹H NMR (500 MHz, Methanol-d4) δ 8.34-8.20 (m, 1H), 7.40 (dd, J=2.0, 0.9 Hz, 1H), 7.29 (s, 1H), 7.16 (dd, J=8.0, 1.4 Hz, 2H), 7.11-7.03 (m, 2H), 7.02-6.96 (m, 1H), 6.78 (dd, J=7.2, 2.0 Hz, 1H), 6.49 (d, J=0.9 Hz, 1H), 3.94-3.79 (m, 2H), 3.70 (s, 3H), 3.38-3.27 (m, 1H), 3.24-3.22 (m, 1H), 2.82-2.71 (m, 1H), 2.57-2.44 (m, 1H), 2.26-2.18 (m, 1H), 2.16 (s, 3H), 1.99-1.77 (m, 2H), 1.73-1.61 (m, 1H), 1.31-1.20 (m, 1H).

### Example 160: 5-[4-[[(2R)-1-ethylazetidin-2-yl]methoxy]-2-methyl-pyrazol-3-yl]-N-[5-(trifluoromethyl)pyrazin-2-yl]pyrazolo[1,5-a]pyridin-2-amine

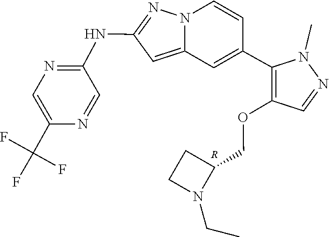

The title compound (42 mg, 3 steps, 14%) was prepared as described in Example 2, except 2-bromo-5-(trifluoromethyl)pyrazine used instead of 4-chloro-2,6-dimethylpyrimidine in Step A and acetaldehyde was used instead of paraformaldehyde in Step C. MS (ESI): mass calcd. for C₂₂H₂₃F₃N₈O, 472.2. m/z found, 473.1 [M+H]⁺. ¹H NMR (400 MHz, CDCl₃) δ 8.77-8.71 (m, 1H), 8.59-8.52 (m, 1H), 8.40-8.34 (m, 1H), 8.13-8.06 (m, 1H), 7.55-7.47 (m, 1H), 7.39-7.33 (m, 1H), 6.93-6.86 (m, 1H), 6.83-6.76 (m, 1H), 4.08-3.95 (m, 2H), 3.95-3.88 (m, 3H), 3.49-3.34 (m, 2H), 2.84-2.74 (m, 1H), 2.74-2.64 (m, 1H), 2.42-2.31 (m, 1H), 2.11-1.94 (m, 2H), 1.00-0.90 (m, 3H) 19F NMR (376 MHz, CDCl₃) δ −66.69 (s, 3F) 1H NMR (400 MHz, DMSO-d6) δ 11.01 (s, 1H), 8.71-8.61 (m, 3H), 7.75 (d, J=0.8 Hz, 1H), 7.46 (s, 1H), 7.03 (s, 1H), 6.99 (dd, J=2.0, 7.2 Hz, 1H), 3.96-3.91 (m, 2H), 3.87 (s, 3H), 3.31-3.19 (m, 2H), 2.71-2.58 (m, 2H), 2.28-2.19 (m, 1H), 2.01-1.90 (m, 1H), 1.90-1.78 (m, 1H), 0.82 (t, J=7.2 Hz, 3H)

### Example 161: 5-[4-[[(2R)-1-ethylazetidin-2-yl]methoxy]-2-methyl-pyrazol-3-yl]-N-[2-(trifluoromethyl)pyrimidin-4-yl]pyrazolo[1,5-a]pyridin-2-amine

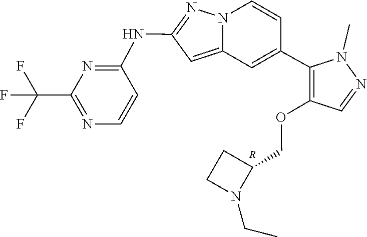

The title compound (135 mg, 3 steps, 34%) was prepared as described in Example 121, except 4-chloro-2-(trifluoromethyl)pyrimidine was used instead of 6-chloro-3-methoxy-4-methylpyridazine in Step A and acetaldehyde was used instead of paraformaldehyde in Step C. MS (ESI): mass calcd. for C₂₂H₂₃F₃N₈O, 472.2. m/z found, 473.1 [M+H]⁺. ¹H NMR (400 MHz, DMSO-d6) δ 11.09 (s, 1H), 8.68 (d, J=6.8 Hz, 1H), 8.57 (d, J=5.2 Hz, 1H), 7.79 (s, 1H), 7.67-7.25 (m, 2H), 7.10-6.72 (m, 2H), 3.98-3.91 (m, 2H), 3.88 (s, 3H), 3.34-3.19 (m, 2H), 2.69-2.57 (m, 2H), 2.30-2.17 (m, 1H), 2.01-1.90 (m, 1H), 1.90-1.78 (m, 1H), 0.83 (t, J=7.2 Hz, 3H) 1H NMR (400 MHz, DMSO-d6, t=80° C.) δ 10.78 (s, 1H), 8.59 (d, J=7.2 Hz, 1H), 8.53 (d, J=6.0 Hz, 1H), 7.72 (s, 1H), 7.54 (d, J=5.6 Hz, 1H), 7.40 (s, 1H), 6.97 (dd, J=2.0, 7.6 Hz, 1H), 6.85 (s, 1H), 3.97-3.88 (m, 2H), 3.85 (s, 3H), 3.32-3.21 (m, 2H), 2.73-2.64 (m, 1H), 2.58-2.53 (m, 1H), 2.33-2.22 (m, 1H), 2.01-1.91 (m, 1H), 1.90-1.78 (m, 1H), 0.83 (t, J=7.2 Hz, 3H) 19F NMR (376 MHz, DMSO-d6) δ −69.77 (s, 3F)

### Example 162: 5-[4-[[(2R)-1-ethylazetidin-2-yl]methoxy]-2-methyl-pyrazol-3-yl]-N-[2-methyl-6-(trifluoromethyl)pyrimidin-4-yl]pyrazolo[1,5-a]pyridin-2-amine

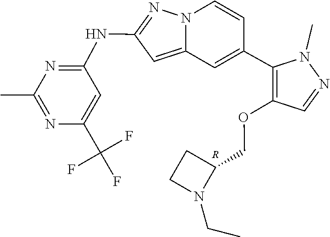

The title compound (26.5 mg, 3 steps, 10%) was prepared as described in Example 121, except 4-chloro-2-methyl-6-(trifluoromethyl)pyrimidine was used instead of 6-chloro-3-methoxy-4-methylpyridazine in Step A and acetaldehyde was used instead of paraformaldehyde in Step C. MS (ESI): mass calcd. for C₂₃H₂₅F₃N₈O, 486.2. m/z found, 487.2 [M+H]⁺. ¹H NMR (400 MHz, CDCl₃) δ 8.41 (d, J=7.2 Hz, 1H), 8.00 (s, 1H), 7.57 (s, 1H), 7.51 (s, 1H), 7.35 (s, 1H), 6.90 (dd, J=1.6, 7.2 Hz, 1H), 6.62 (s, 1H), 4.06-3.95 (m, 2H), 3.92 (s, 3H), 3.47-3.34 (m, 2H), 2.82-2.74 (m, 1H), 2.74-2.63 (m, 4H), 2.42-2.30 (m, 1H), 2.11-1.93 (m, 2H), 0.95 (t, J=7.2 Hz, 3H) 19F NMR (376 MHz, DMSO-d6) δ −69.32 (s, 3F)

### Example 163: 5-[4-[[(2R)-1-ethylazetidin-2-yl]methoxy]-2-methyl-pyrazol-3-yl]-N-(2-methoxypyrimidin-4-yl)pyrazolo[1,5-a]pyridin-2-amine

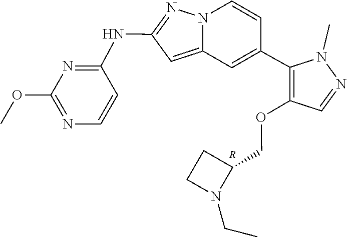

The title compound (44.6 mg, 3 steps, 13%) was prepared as described in Example 139 (Steps E-G) except 4-chloro-2-methoxypyrimidine was used instead of 4-chloro-6-ethyl-2-methylpyrimidine in Step E and Intermediate 6 was used instead of (S)-tert-butyl 2-(((5-(2-aminopyrazolo[1,5-a]pyridin-5-yl)-1-methyl-1H-pyrazol-4-yl)oxy)methyl)azetidine-1-carboxylate in Step E. In Step G acetaldehyde was used instead of paraformaldehyde. MS (ESI): mass calcd. for C₂₂H₂₆N₈O₂, 434.2. m/z found, 435.1 [M+H]⁺. ¹H NMR (400 MHz, DMSO-d6) δ 10.50 (s, 1H), 8.62 (d, J=7.2 Hz, 1H), 8.17 (d, J=6.0 Hz, 1H), 7.73 (d, J=1.2 Hz, 1H), 7.46 (s, 1H), 7.05-6.91 (m, 2H), 6.85 (br s, 1H), 4.02-3.94 (m, 2H), 3.91 (s, 3H), 3.87 (s, 3H), 3.35-3.27 (m, 2H), 2.70-2.55 (m, 2H), 2.38-2.26 (m, 1H), 2.06-1.93 (m, 1H), 1.92-1.80 (m, 1H), 0.84 (t, J=7.2 Hz, 3H) 1H NMR (400 MHz, CD3OD) δ 8.47 (d, J=7.2 Hz, 1H), 8.09 (d, J=6.0 Hz, 1H), 7.65 (d, J=0.8 Hz, 1H), 7.45 (s, 1H), 6.98 (br s, 1H), 6.94 (dd, J=2.0, 7.2 Hz, 1H), 6.85 (br s, 1H), 4.09-4.04 (m, 2H), 4.03 (s, 3H), 3.89 (s, 3H), 3.70-3.59 (m, 1H), 3.49-3.42 (m, 1H), 2.99 (q, J=8.6 Hz, 1H), 2.84-2.71 (m, 1H), 2.55-2.42 (m, 1H), 2.20-2.09 (m, 1H), 2.09-1.98 (m, 1H), 0.94 (t, J=7.2 Hz, 3H)

### Example 164: 5-[4-[[(2R)-1-ethylazetidin-2-yl]methoxy]-2-methyl-pyrazol-3-yl]-N-[6-(trifluoromethyl)pyrimidin-4-yl]pyrazolo[1,5-a]pyridin-2-amine

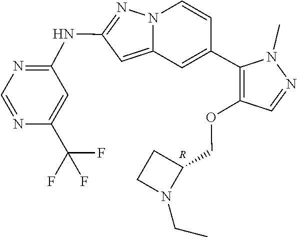

The title compound (53.5 mg, 3 steps, 22%) was prepared as described in Example 121 except 2-chloro-5-fluoropyrimidine was used instead of 4-chloro-6-ethyl-2-methylpyrimidine in Step A and acetaldehyde was used instead of paraformaldehyde in Step C. MS (ESI): mass calcd. for C₂₂H₂₃F₃N₈O, 472.2. m/z found, 473.2 [M+H]⁺. ¹H NMR (400 MHz, CDCl₃) δ 8.89 (s, 1H), 8.41 (d, J=7.2 Hz, 1H), 8.31 (s, 1H), 7.80 (br. s., 1H), 7.52 (d, J=0.8 Hz, 1H), 7.36 (s, 1H), 6.91 (dd, J=2.0, 7.2 Hz, 1H), 6.62 (br. s., 1H), 4.17-4.06 (m, 1H), 4.04-3.96 (m, 1H), 3.92 (s, 3H), 3.58-3.44 (m, 2H), 2.93-2.81 (m, 1H), 2.79-2.67 (m, 1H), 2.49-2.37 (m, 1H), 2.15-2.02 (m, 2H), 0.98 (t, J=7.2 Hz, 3H) 19F NMR (376 MHz, CDCl₃) δ −70.38 (s, 3F)

### Example 165: N-(4,5-dimethylpyrimidin-2-yl)-5-[4-[[(2R)-1-ethylazetidin-2-yl]methoxy]-2-methyl-pyrazol-3-yl]pyrazolo[1,5-a]pyridin-2-amine

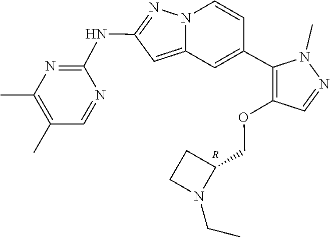

The title compound (32.8 mg, 3 steps, 11%) was prepared as described in Example 121, except 2-chloro-4,5-dimethylpyrimidine was used instead of 4-chloro-6-ethyl-2-methylpyrimidine in Step A and acetaldehyde was used instead of paraformaldehyde in Step C. MS (ESI): mass calcd. for C₂₃H₂₈N₈O, 432.2. m/z found, 433.1 [M+H]⁺. ¹H NMR (400 MHz, DMSO-d6) δ 10.00 (s, 1H), 8.55 (d, J=7.2 Hz, 1H), 8.22 (s, 1H), 7.67 (d, J=0.8 Hz, 1H), 7.44 (s, 1H), 7.14 (s, 1H), 6.89 (dd, J=1.6, 7.2 Hz, 1H), 3.95-3.91 (m, 2H), 3.86 (s, 3H), 3.32-3.21 (m, 2H), 2.69-2.63 (m, 1H), 2.61-2.55 (m, 1H), 2.38 (s, 3H), 2.28-2.19 (m, 1H), 2.14 (s, 3H), 1.99-1.91 (m, 1H), 1.88-1.74 (m, 1H), 0.82 (t, J=7.2 Hz, 3H)

### Example 166: 5-[4-[[(2R)-1-ethylazetidin-2-yl]methoxy]-2-methyl-pyrazol-3-yl]-N-(5-methoxy-2-pyridyl)pyrazolo[1,5-a]pyridin-2-amine

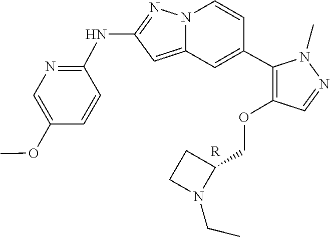

The title compound (80 mg, 3 steps, 38%) was prepared as described in Example 138 (Steps E-G), except 2-bromo-5-methoxypyridine was used instead of 4-chloro-6-ethyl-2-methylpyrimidine in Step E and Intermediate 6 was used instead of (S)-tert-butyl 2-(((5-(2-aminopyrazolo[1,5-a]pyridin-5-yl)-1-methyl-1H-pyrazol-4-yl)oxy)methyl)azetidine-1-carboxylate in Step E. In Step G acetaldehyde was used instead of paraformaldehyde. MS (ESI): mass calcd. for C₂₃H₂₇N₇O₂, 433.2. m/z found, 434.1 [M+H]⁺. ¹H NMR (400 MHz, DMSO-d6) δ 9.56 (s, 1H), 8.54 (d, J=7.2 Hz, 1H), 7.94 (d, J=0.8 Hz, 1H), 7.60 (s, 1H), 7.44 (s, 1H), 7.39-7.31 (m, 2H), 6.84 (d, J=7.6 Hz, 1H), 6.76 (s, 1H), 3.99-3.92 (m, 2H), 3.85 (s, 3H), 3.78 (s, 3H), 3.32-3.16 (m, 2H), 2.79-2.57 (m, 2H), 2.36-2.23 (m, 1H), 2.04-1.93 (m, 1H), 1.91-1.80 (m, 1H), 0.84 (t, J=7.2 Hz, 3H)

### Example 167: 5-[4-[[(2R)-1-ethylazetidin-2-yl]methoxy]-2-methyl-pyrazol-3-yl]-N-(6-methoxypyrimidin-4-yl)pyrazolo[1,5-a]pyridin-2-amine

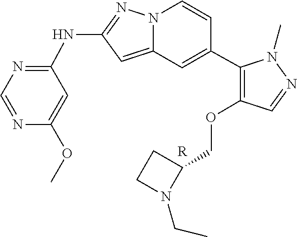

The title compound (42 mg, 3 steps, 20%) was prepared as described in Example 138 (Steps E-G), except 4-chloro-6-methoxypyrimidine was used instead of 4-chloro-6-ethyl-2-methylpyrimidine in Step E and Intermediate 6 was used instead of (S)-tert-butyl 2-(((5-(2-aminopyrazolo[1,5-a]pyridin-5-yl)-1-methyl-1H-pyrazol-4-yl)oxy)methyl)azetidine-1-carboxylate in Step E. In Step G acetaldehyde was used instead of paraformaldehyde. MS (ESI): mass calcd. for C₂₂H₂₆N₈O₂, 434.2. m/z found, 435.1 [M+H]⁺. ¹H NMR (400 MHz, CDCl₃) δ 8.48 (s, 1H), 8.38 (d, J=6.8 Hz, 1H), 7.78 (s, 1H), 7.45 (s, 1H), 7.34 (s, 1H), 6.90 (s, 1H), 6.84 (dd, J=1.6, 7.2 Hz, 1H), 6.43 (s, 1H), 4.03-3.94 (m, 5H), 3.91 (s, 3H), 3.46-3.33 (m, 2H), 2.82-2.72 (m, 1H), 2.71-2.62 (m, 1H), 2.40-2.30 (m, 1H), 2.08-1.91 (m, 2H), 0.94 (t, J=7.2 Hz, 3H) 1H NMR (400 MHz, DMSO-d6) δ 10.22 (s, 1H), 8.63 (d, J=6.8 Hz, 1H), 8.42 (s, 1H), 7.66 (s, 1H), 7.44 (s, 1H), 6.95-6.87 (m, 2H), 6.67 (s, 1H), 3.95-3.91 (m, 2H), 3.89 (s, 3H), 3.86 (s, 3H), 3.29-3.19 (m, 2H), 2.69-2.55 (m, 2H), 2.28-2.19 (m, 1H), 2.02-1.90 (m, 1H), 1.88-1.78 (m, 1H), 0.82 (t, J=7.6 Hz, 3H)

### Example 168: 5-[4-[[(2S)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine

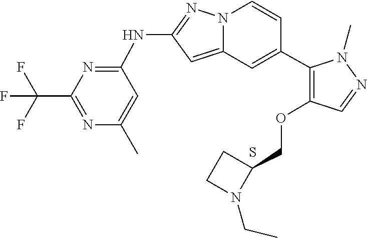

Step A. Tert-butyl (S)-2-((tosyloxy)methyl)azetidine-1-carboxylate. A solution consisting of (S)-1-Boc-2-azetidinemethanol (6.00 g, 32.0 mmol, 1 equiv.), DMAP (5.87 g, 48.1 mmol, 1.5 equiv.), and DCM (60 mL) was treated with TsCl (9.16 g, 48.1 mmol, 1.5 equiv.). The resultant mixture was stirred overnight at room-temperature. The reaction mixture was diluted with ethyl acetate (60 mL) and washed with 1N HCl (100 mL), saturated aqueous NaHCO₃ (100 mL), and brine (100 mL) in sequence. The organic extract was dried over anhydrous Na₂SO₄, filtered, and evaporated to dryness under reduced pressure to give the crude product (12.2 g). The obtained crude product was purified by FCC (eluent: petroleum ether:ethyl acetate=100:0 to 70:30) to afford the title compound as a colorless oil (9.4 g, 86% yield).

Step B. Tert-butyl (S)-2-(((1-methyl-1H-pyrazol-4-yl)oxy)methyl)azetidine-1-carboxylate. A mixture consisting of 1-methyl-1H-pyrazol-4-ol (2.60 g, 26.5 mmol, 1 equiv.), (S)-tert-butyl 2-((tosyloxy)methyl)azetidine-1-carboxylate (9.05 g, 26.5 mmol, 1 equiv.), CS2CO3 (25.9 g, 79.5 mmol) and DMF (30 mL) was stirred at 60° C. for 16 h. The reaction mixture was filtered and purified by FCC (petroleum ether:ethyl acetate=1:0 to 0:1) to afford product (5.96 g, 84%) as a yellow oil.

Step C. Tert-butyl (S)-2-(((5-bromo-1-methyl-1H-pyrazol-4-yl)oxy)methyl)azetidine-1-carboxylate. A mixture consisting of (S)-tert-butyl 2-(((1-methyl-1H-pyrazol-4-yl)oxy)methyl)azetidine-1-carboxylate (5.96 g, 22.3 mmol, 1 equiv.), NBS (3.18 g, 17.8 mmol, 1 equiv.) and MeCN (60 mL) was stirred at room temperature for 1 hr. The reaction mixture was poured into water (300 mL) and concentrated under reduced pressure to remove solvent and extracted with EtOAc (300 mL×3). The combined organic extracts were washed with brine (100 mL×3), dried over anhydrous Na₂SO₄, filtered, and concentrated to dryness under reduced pressure to give the crude product, which was purified by FCC (eluent: petroleum ether:ethyl acetate=1:0 to 1:1) to afford the title compound (5.1 g, 66%) as a yellow oil.

Step D. Tert-butyl (S)-2-(((5-(2-aminopyrazolo[1,5-a]pyridin-5-yl)-1-methyl-1H-pyrazol-4-yl)oxy)methyl)azetidine-1-carboxylate. A mixture consisting of Intermediate 5 (4.20 g, 16.2 mmol, 1.1 equiv.), (S)-tert-butyl 2-(((5-bromo-1-methyl-1H-pyrazol-4-yl)oxy)methyl)azetidine-1-carboxylate (5.1 g, 15 mmol, 1 equiv.), Pd(dppf)Cl₂ (1.08 g, 1.47 mmol, 0.1 equiv.), potassium phosphate (7.82 g, 36.8 mmol, 2.5 equiv.), 1,4-dioxane/H₂O (50 mL, 4:1) under N2 was stirred at 90° C. for 4 h. The mixture was concentrated under reduced pressure to give a crude product, which was purified by FCC (eluent: petroleum ether:ethyl acetate=1:0 to 0:1) to afford the title compound (5.2 g, 81%) as a yellow oil.

Step E. 5-[4-[[(2S)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine. The title compound (36 mg, 2 steps, 29%) was prepared as described in Example 518 (Steps E-G), except 4-chloro-6-methyl-2-(trifluoromethyl)pyrimidine was used instead of 4-chloro-6-ethyl-2-methylpyrimidine in Step E and Intermediate 6 (1 equiv.) was used instead of (S)-tert-butyl 2-(((5-(2-aminopyrazolo[1,5-a]pyridin-5-yl)-1-methyl-1H-pyrazol-4-yl)oxy)methyl)azetidine-1-carboxylate in Step E. In Step G acetaldehyde (1 equiv.) was used instead of paraformaldehyde. MS (ESI): mass calcd. for C₂₃H₂₅F₃N₈O, 486.2. m/z found, 487.1 [M+H]⁺. ¹H NMR (400 MHz, DMSO-d6) δ 10.93 (s, 1H), 8.67 (d, J=7.2 Hz, 1H), 7.76 (s, 1H), 7.54-7.16 (m, 2H), 6.99 (dd, J=2.0, 7.2 Hz, 1H), 6.87 (s, 1H), 3.95-3.91 (m, 2H), 3.88 (s, 3H), 3.28-3.20 (m, 2H), 2.69-2.65 (m, 1H), 2.63-2.59 (m, 1H), 2.45 (s, 3H), 2.27-2.17 (m, 1H), 1.99-1.90 (m, 1H), 1.89-1.78 (m, 1H), 0.82 (t, J=7.2 Hz, 3H) 1H NMR (400 MHz, CDCl₃) δ 8.42 (d, J=7.2 Hz, 1H), 7.83 (s, 1H), 7.51 (d, J=0.8 Hz, 1H), 7.41-7.30 (m, 2H), 6.90 (dd, J=2.0, 7.2 Hz, 1H), 6.64 (s, 1H), 4.06-4.00 (m, 1H), 4.00-3.95 (m, 1H), 3.92 (s, 3H), 3.46-3.35 (m, 2H), 2.83-2.74 (m, 1H), 2.73-2.62 (m, 1H), 2.57 (s, 3H), 2.43-2.30 (m, 1H), 2.08-1.92 (m, 2H), 0.95 (t, J=7.2 Hz, 3H) 19F NMR (376 MHz, CDCl3) δ −71.0 (s, 3F)

### Example 169: 5-[4-[[(2S)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine

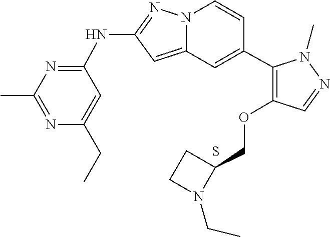

The title compound (550 mg, 2 steps, 29%) was prepared as described in Example 138 (Steps E-G), except (S)-tert-butyl 2-(((5-(2-aminopyrazolo[1,5-a]pyridin-5-yl)-1-methyl-1H-pyrazol-4-yl)oxy)methyl)azetidine-1-carboxylate was used instead of Intermediate 6 in Step E and 4-chloro-6-ethyl-2-methylpyrimidine was used instead of 4-chloro-6-ethyl-2-methylpyrimidine in Step E. In Step G acetaldehyde was used instead of paraformaldehyde. MS (ESI): mass calcd. for C₂₄H₃₀N₈O, 446.3. m/z found, 447.1 [M+H]⁺. ¹H NMR (400 MHz, DMSO-d6) δ 10.23 (s, 1H), 8.62 (d, J=7.2 Hz, 1H), 7.72 (d, J=0.8 Hz, 1H), 7.48 (s, 1H), 7.00 (br. s., 1H), 6.95 (dd, J=1.6, 6.8 Hz, 1H), 6.93 (br. s., 1H), 4.17-4.02 (m, 2H), 3.88 (s, 3H), 3.82-3.74 (m, 1H), 3.73-3.66 (m, 1H), 3.16-3.03 (m, 1H), 2.86-2.73 (m, 1H), 2.62-2.54 (m, 3H), 2.48 (s, 3H), 2.19-2.08 (m, 1H), 2.05-1.96 (m, 1H), 1.20 (t, J=8.0 Hz, 3H), 0.92 (t, J=7.2 Hz, 3H) 1H NMR (400 MHz, CD3OD) δ 8.49 (d, J=7.2 Hz, 1H), 7.64 (d, J=0.8 Hz, 1H), 7.47 (s, 1H), 7.01 (s, 1H), 6.94 (br. s., 1H), 6.92 (dd, J=2.0, 7.6 Hz, 1H), 4.19-4.13 (m, 2H), 4.03-3.93 (m, 1H), 3.89 (s, 3H), 3.72-3.63 (m, 1H), 3.30-3.25 (m, 1H), 2.99-2.88 (m, 1H), 2.77-2.69 (m, 1H), 2.65 (q, J=7.6 Hz, 2H), 2.54 (s, 3H), 2.32-2.24 (m, 1H), 2.24-2.12 (m, 1H), 1.28 (t, J=8.0 Hz, 3H), 1.01 (t, J=7.6 Hz, 3H)

### Example 170: 6-[[5-[4-[[(2R)-1-ethylazetidin-2-yl]methoxy]-2-methyl-pyrazol-3-yl]pyrazolo[1,5-a]pyridin-2-yl]amino]-2-methyl-pyridazin-3-one

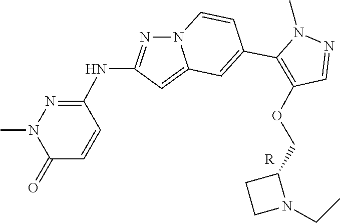

The title compound (76 mg, 3 steps, 45%) was prepared as described in Example 2, except 6-bromo-2-methyl-3(2h)-pyridazinone was used instead of 4-chloro-2,6-dimethylpyrimidine in Step A and acetaldehyde was used instead of paraformaldehyde in Step C. MS (ESI): mass calcd. for C₂₂H₂₆N₈O₂, 434.2. m/z found, 435.2 [M+H]⁺. ¹H NMR (500 MHz, Chloroform-d) δ 8.22 (dt, J=7.2, 0.9 Hz, 1H), 8.10 (d, J=3.5 Hz, 1H), 7.40 (dd, J=1.9, 0.9 Hz, 1H), 7.29-7.19 (m, 2H), 6.89 (d, J=9.7 Hz, 1H), 6.75 (dd, J=7.2, 1.9 Hz, 1H), 6.67 (d, J=0.9 Hz, 1H), 3.99-3.84 (m, 2H), 3.83 (s, 3H), 3.70 (s, 3H), 3.37-3.25 (m, 2H), 2.71-2.66 (m, 1H), 2.64-2.56 (m, 1H), 2.31-2.23 (m, 1H), 2.01-1.84 (m, 2H), 0.86 (t, J=7.3 Hz, 3H).

### Example 171: N-(2-cyclopropyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine

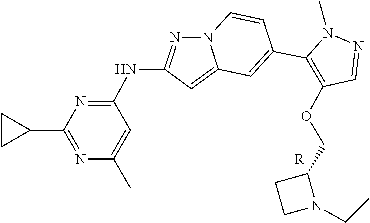

The title compound (111 mg, 3 steps, 58%) was prepared as described in Example 2, except 4-chloro-2-cyclopropyl-6-methylpyrimidine was used instead of 4-chloro-2,6-dimethylpyrimidine in Step A and acetaldehyde was used instead of paraformaldehyde in Step C. MS (ESI): mass calcd. for C₂₅H₃₀N₈O, 458.3. m/z found, 459.2 [M+H]⁺. ¹H NMR (400 MHz, Chloroform-d) δ 8.42-8.30 (m, 2H), 7.47 (dd, J=2.0, 0.9 Hz, 1H), 7.34 (s, 1H), 6.86-6.76 (m, 2H), 6.68 (s, 1H), 4.06-3.91 (m, 2H), 3.91 (s, 3H), 3.49-3.30 (m, 2H), 2.81-2.60 (m, 2H), 2.38 (s, 3H), 2.39-2.27 (m, 1H), 2.13 (tt, J=8.1, 4.7 Hz, 1H), 2.09-1.97 (m, 1H), 2.01-1.89 (m, 1H), 1.16 (dt, J=4.8, 3.1 Hz, 2H), 1.06-0.89 (m, 5H).

### Example 172: 5-[4-[[(2R)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine

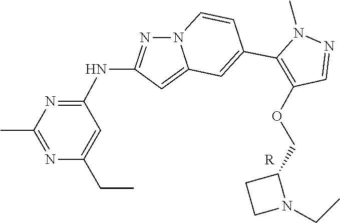

The title compound (75 mg, 3 steps, 38%) was prepared as described in Example 2, except 4-chloro-6-ethyl-2-methylpyrimidine was used instead of 4-chloro-2,6-dimethylpyrimidine in Step A and acetaldehyde was used instead of paraformaldehyde in Step C. MS (ESI): mass calcd. for C₂₄H₃₀N₈O, 446.3. m/z found, 447.3 [M+H]⁺. ¹H NMR (500 MHz, Chloroform-d) δ 8.45 (dt, J=7.2, 0.9 Hz, 1H), 8.04-7.99 (m, 1H), 7.48 (dd, J=1.9, 0.9 Hz, 1H), 7.36 (s, 1H), 7.01 (s, 1H), 6.86 (dd, J=7.2, 1.9 Hz, 1H), 6.64 (s, 1H), 4.03 (dd, J=9.8, 6.5 Hz, 1H), 3.97 (dd, J=9.8, 4.8 Hz, 1H), 3.92 (s, 3H), 3.46-3.33 (m, 2H), 2.82-2.64 (m, 4H), 2.63 (s, 3H), 2.36 (dq, J=11.4, 7.1 Hz, 1H), 2.09-1.92 (m, 2H), 1.31 (t, J=7.6 Hz, 3H), 0.95 (t, J=7.2 Hz, 3H).

### Example 173: 5-[4-[[(2R)-1-ethylazetidin-2-yl]methoxy]-2-methyl-pyrazol-3-yl]-N-(2-methoxy-6-methyl-pyrimidin-4-yl)pyrazolo[1,5-a]pyridin-2-amine

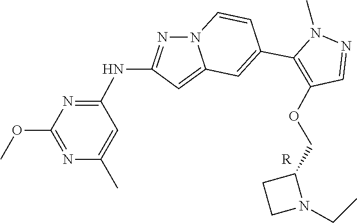

The title compound (37 mg, 3 steps, 46%) was prepared as described in Example 2, except 4-chloro-2-methoxy-6-methylpyrimidine was used instead of 4-chloro-2,6-dimethylpyrimidine in Step A and acetaldehyde was used instead of paraformaldehyde in Step C. MS (ESI): mass calcd. for C₂₃H₂₈N₈O₂, 448.2. m/z found, 449.2 [M+H]⁺. ¹H NMR (500 MHz, Chloroform-d) δ 8.37 (dt, J=7.2, 0.9 Hz, 1H), 7.60 (s, 1H), 7.49 (dd, J=2.0, 1.0 Hz, 1H), 7.36 (s, 1H), 6.86 (dd, J=7.2, 1.9 Hz, 1H), 6.73 (s, 2H), 4.03 (s, 3H), 4.07-3.99 (m, 1H), 3.97 (dd, J=9.8, 4.8 Hz, 1H), 3.93 (s, 3H), 3.46-3.33 (m, 2H), 2.81-2.75 (m, 1H), 2.72-2.64 (m, 1H), 2.44-2.31 (m, 4H), 2.10-1.93 (m, 2H), 0.96 (t, J=7.2 Hz, 3H).

### Example 174: N-[5-[4-[[(2R)-1-ethylazetidin-2-yl]methoxy]-2-methyl-pyrazol-3-yl]pyrazolo[1,5-a]pyridin-2-yl]-2-methyl-imidazo[1,2-b]pyridazin-6-amine

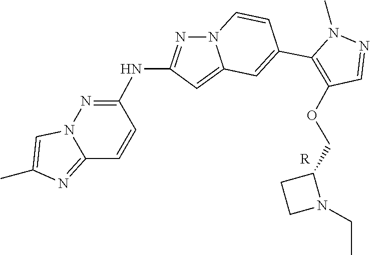

The title compound (37 mg, 3 steps, 53%) was prepared as described in Example 2, except 6-chloro-2-methylimidazo[1,2-b]pyridazine was used instead of 4-chloro-2,6-dimethylpyrimidine in Step A and acetaldehyde was used instead of paraformaldehyde in Step C. MS (ESI): mass calcd. for C₂₄H₂₇N₉O, 457.2. m/z found, 458.2 [M+H]⁺. ¹H NMR (500 MHz, Chloroform-d) δ 9.14 (d, J=10.7 Hz, 1H), 8.26 (dd, J=7.1, 1.0 Hz, 1H), 7.55 (d, J=9.5 Hz, 1H), 7.49 (s, 1H), 7.42 (dd, J=2.0, 0.9 Hz, 1H), 7.26 (s, 1H), 6.92 (d, J=0.9 Hz, 1H), 6.77-6.71 (m, 2H), 3.97-3.85 (m, 2H), 3.82 (s, 3H), 3.40-3.24 (m, 3H), 2.71-2.55 (m, 2H), 2.36 (s, 3H), 2.31-2.22 (m, 1H), 1.99-1.84 (m, 1H), 0.86 (t, J=7.2 Hz, 3H).

### Example 175: N-[5-[4-[[(2R)-1-ethylazetidin-2-yl]methoxy]-2-methyl-pyrazol-3-yl]pyrazolo[1,5-a]pyridin-2-yl]imidazo[1,2-b]pyridazin-6-amine

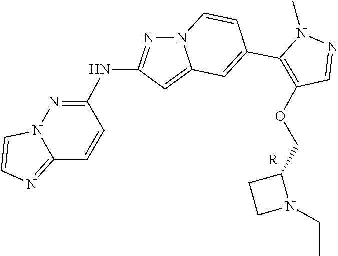

The title compound (133 mg, 3 steps, 69%) was prepared as described in Example 2, except 6-chloroimidazo[1,2-b]pyridazine was used instead of 4-chloro-2,6-dimethylpyrimidine in Step A and acetaldehyde was used instead of paraformaldehyde in Step C. MS (ESI): mass calcd. for C₂₃H₂₅N₉O, 443.2. m/z found, 444.2 [M+H]⁺. ¹H NMR (400 MHz, Chloroform-d) δ 8.35 (dt, J=7.2, 0.9 Hz, 1H), 8.24 (d, J=2.1 Hz, 1H), 7.85-7.78 (m, 2H), 7.64 (d, J=1.1 Hz, 1H), 7.53 (dd, J=2.0, 0.9 Hz, 1H), 7.37 (s, 1H), 7.03 (d, J=0.8 Hz, 1H), 6.93-6.83 (m, 2H), 4.09-3.94 (m, 2H), 3.93 (s, 3H), 3.52-3.34 (m, 3H), 2.84-2.64 (m, 2H), 2.42-2.32 (m, 1H), 2.05-1.93 (m, 1H), 0.97 (t, J=7.2 Hz, 3H).

### Example 176: (R)-5-(4-((1-ethylazetidin-2-yl)methoxy)-1-methyl-1H-pyrazol-5-yl)-N-(5-fluoropyrimidin-2-yl)pyrazolo[1,5-a]pyridin-2-amine

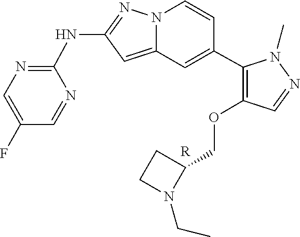

The title compound (550 mg, 3 steps, 3%) was prepared as described in Example 121, except 2-chloro-5-fluoropyrimidine was used instead of 6-chloro-3-methoxy-4-methylpyridazine in Step A and acetaldehyde was used instead of paraformaldehyde in Step C. MS (ESI): mass calcd. for C₂₁H₂₃FN₈O, 422.2. m/z found, 423.2 [M+H]⁺. 1H NMR (400 MHz, DMSO-d6) δ 10.48 (s, 1H), 8.62 (s, 2H), 8.58 (d, J=7.2 Hz, 1H), 7.68 (d, J=0.8 Hz, 1H), 7.44 (s, 1H), 7.06 (s, 1H), 6.92 (dd, J=2.0, 7.6 Hz, 1H), 3.95-3.90 (m, 2H), 3.86 (s, 3H), 3.34-3.18 (m, 2H), 2.69-2.55 (m, 2H), 2.28-2.18 (m, 1H), 1.99-1.91 (m, 1H), 1.87-1.74. 19F NMR (376 MHz, DMSO-d6) δ −152.21 (s, 1F).

### Example 177: N-(2-tert-butyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine

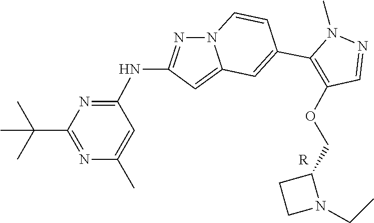

The title compound (104 mg, 3 steps, 77%) was prepared as described in Example 2, except 2-tert-butyl-4-chloro-6-methylpyrimidine was used instead of 4-chloro-2,6-dimethylpyrimidine in Step A and acetaldehyde was used instead of paraformaldehyde in Step C. MS (ESI): mass calcd. for C₂₆H₃₄N₈O, 474.3. m/z found, 475.3 [M+H]⁺. ¹H NMR (500 MHz, Chloroform-d) δ 8.32-8.26 (m, 1H), 7.91 (d, J=15.5 Hz, 1H), 7.44-7.39 (m, 1H), 7.27 (s, 1H), 6.80-6.71 (m, 2H), 6.65 (s, 1H), 3.94 (dd, J=9.8, 6.5 Hz, 1H), 3.88 (dd, J=9.8, 4.7 Hz, 1H), 3.84 (s, 3H), 3.37-3.24 (m, 2H), 2.72-2.65 (m, 1H), 2.62-2.55 (m, 1H), 2.33 (s, 3H), 2.31-2.22 (m, 1H), 2.00-1.91 (m, 1H), 1.94-1.84 (m, 1H), 1.34 (s, 9H), 0.86 (t, J=7.2 Hz, 3H).

### Example 178: 6-[[5-[4-[[(2R)-1-ethylazetidin-2-yl]methoxy]-2-methyl-pyrazol-3-yl]pyrazolo[1,5-a]pyridin-2-yl]amino]-2,4-dimethyl-pyridazin-3-one

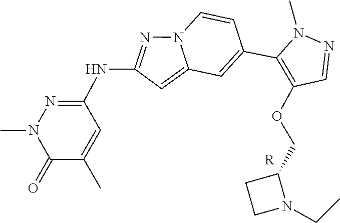

The title compound (36 mg, 3 steps, 51%) was prepared as described in Example 2, except 6-chloro-2,4-dimethylpyridazin-3(2h)-one was used instead of 4-chloro-2,6-dimethylpyrimidine in Step A and acetaldehyde was used instead of paraformaldehyde in Step C. MS (ESI): mass calcd. for C₂₃H₂₈N₈O₂, 448.2. m/z found, 449.2 [M+H]⁺. ¹H NMR (500 MHz, Chloroform-d) δ 8.31 (dt, J=7.1, 0.9 Hz, 1H), 7.47 (dd, J=1.9, 0.9 Hz, 1H), 7.36 (s, 1H), 7.26 (d, J=6.1 Hz, 1H), 7.11 (q, J=1.2 Hz, 1H), 6.83 (dd, J=7.2, 1.9 Hz, 1H), 6.71 (d, J=0.8 Hz, 1H), 5.31 (s, OH), 4.03 (dd, J=9.8, 6.5 Hz, 1H), 3.97 (dd, J=9.8, 4.8 Hz, 1H), 3.92 (s, 2H), 3.79 (s, 3H), 3.50 (s, 1H), 3.46-3.34 (m, 2H), 2.81-2.75 (m, 1H), 2.73-2.65 (m, 1H), 2.40-2.32 (m, 1H), 2.26 (d, J=1.3 Hz, 3H), 2.10-1.93 (m, 2H), 0.96 (t, J=7.2 Hz, 3H).

### Example 179: N-[2-(cyclopropylmethyl)-6-methyl-pyrimidin-4-yl]-5-[4-[[(2R)-1-ethylazetidin-2-yl]methoxy]-2-methyl-pyrazol-3-yl]pyrazolo[1,5-a]pyridin-2-amine

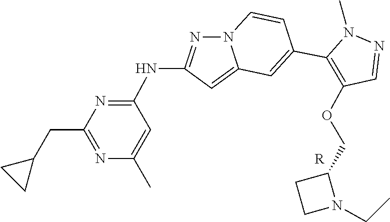

The title compound (118 mg, 3 steps, 78%) was prepared as described in Example 2, except 4-chloro-2-(cyclopropylmethyl)-6-methylpyrimidine was used instead of 4-chloro-2,6-dimethylpyrimidine in Step A and acetaldehyde was used instead of paraformaldehyde in Step C. MS (ESI): mass calcd. for C₂₆H₃₂N₈O, 472.3. m/z found, 473.3 [M+H]⁺. ¹H NMR (500 MHz, Chloroform-d) δ 8.24 (dt, J=7.2, 0.9 Hz, 1H), 7.50 (d, J=2.8 Hz, 1H), 7.32 (dd, J=1.9, 0.9 Hz, 1H), 7.19 (s, 1H), 6.83 (s, 1H), 6.68 (dd, J=7.2, 1.9 Hz, 1H), 6.54 (s, 1H), 3.87 (dd, J=9.8, 6.5 Hz, 1H), 3.81 (dd, J=9.8, 4.8 Hz, 1H), 3.76 (s, 3H), 3.30-3.17 (m, 2H), 2.60 (dd, J=15.3, 7.7 Hz, 3H), 2.56-2.48 (m, 1H), 2.30 (s, 3H), 2.24-2.16 (m, 1H), 1.94-1.77 (m, 2H), 1.19-1.08 (m, 1H), 0.79 (t, J=7.2 Hz, 3H), 0.44-0.33 (m, 2H), 0.16 (dt, J=5.9, 4.5 Hz, 2H).

### Example 180: 5-[4-[[(2R)-1-ethylazetidin-2-yl]methoxy]-2-methyl-pyrazol-3-yl]-N-(5-methoxypyrazin-2-yl)pyrazolo[1,5-a]pyridin-2-amine

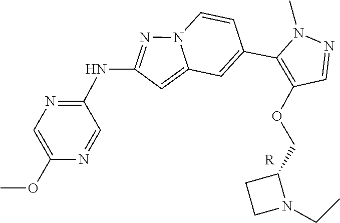

The title compound (68 mg, 3 steps, 44%) was prepared as described in Example 2, except 2-chloro-5-methoxypyrazine was used instead of 4-chloro-2,6-dimethylpyrimidine in Step A and acetaldehyde was used instead of paraformaldehyde in Step C. MS (ESI): mass calcd. for C₂₂H₂₆N₈O₂, 434.2. m/z found, 435.2 [M+H]⁺. ¹H NMR (500 MHz, Chloroform-d) δ 8.47 (d, J=1.5 Hz, 1H), 8.36 (dt, J=7.1, 0.9 Hz, 1H), 7.96 (d, J=1.5 Hz, 1H), 7.48 (s, 1H), 7.41 (dd, J=2.0, 0.9 Hz, 1H), 7.35 (s, 1H), 6.79 (dd, J=7.2, 1.9 Hz, 1H), 6.44 (d, J=0.8 Hz, 1H), 5.31 (s, OH), 4.06-3.93 (m, 2H), 3.97 (s, 3H), 3.91 (s, 3H), 3.49 (s, OH), 3.46-3.33 (m, 2H), 2.81-2.74 (m, 1H), 2.72-2.64 (m, 1H), 2.40-2.32 (m, 1H), 2.10-1.92 (m, 2H), 0.96 (t, J=7.2 Hz, 3H).

### Example 181: N-[5-(difluoromethoxy)pyrazin-2-yl]-5-[4-[[(2R)-1-ethylazetidin-2-yl]methoxy]-2-methyl-pyrazol-3-yl]pyrazolo[1,5-a]pyridin-2-amine

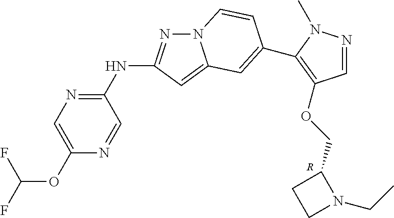

The title compound (98 mg, 3 steps, 50%) was prepared as described in Example 2, except 2-chloro-5-(difluoromethoxy)pyrazine was used instead of 4-chloro-2,6-dimethylpyrimidine in Step A and acetaldehyde was used instead of paraformaldehyde in Step C. MS (ESI): mass calcd. for C₂₂H₂₄F₂N₈O₂, 470.2. m/z found, 471.2 [M+H]⁺. ¹H NMR (400 MHz, Chloroform-d) δ 8.57 (d, J=1.4 Hz, 1H), 8.36 (dt, J=7.1, 0.9 Hz, 1H), 8.07 (d, J=1.4 Hz, 1H), 7.49-7.43 (m, 1H), 7.36 (s, 1H), 7.28 (d, J=6.8 Hz, 1H), 7.10 (s, OH), 6.84 (dd, J=7.2, 1.9 Hz, 1H), 6.49 (d, J=0.7 Hz, 1H), 4.08-3.93 (m, 2H), 3.93 (s, 3H), 3.48-3.33 (m, 2H), 2.83-2.74 (m, 1H), 2.73-2.63 (m, 1H), 2.42-2.32 (m, 1H), 2.12-1.92 (m, 2H), 0.97 (t, J=7.2 Hz, 3H).

### Example 182: N-[5-[2-methyl-4-[[(3R)-pyrrolidin-3-yl]oxymethyl]pyrazol-3-yl]pyrazolo[1,5-a]pyridin-2-yl]cyclopropanecarboxamide

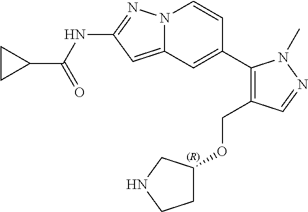

The title compound (60 mg, 2 steps, 11%) was prepared as described in Example 362 Steps A-B. MS (ESI): mass calcd. for C₂₀H₂₄N₆O₂, 380.2. m/z found, 381.1 [M+H]⁺. ¹H NMR (400 MHz, Methanol-d4) δ 8.50 (dt, J=7.1, 1.0 Hz, 1H), 7.64 (dd, J=2.0, 1.0 Hz, 1H), 7.59 (s, 1H), 6.95 (d, J=0.9 Hz, 1H), 6.87 (dd, J=7.2, 1.9 Hz, 1H), 4.33 (s, 2H), 4.13 (tt, J=5.1, 2.8 Hz, 1H), 3.85 (s, 3H), 3.05-2.85 (m, 4H), 1.86 (tdd, J=8.0, 5.5, 3.6 Hz, 3H), 1.00 (dt, J=4.6, 3.1 Hz, 2H), 0.95-0.83 (m, 2H).

### Example 183: N-[5-[2-methyl-4-[[(1S,5R)-3-oxa-9-azabicyclo[3.3.1]nonan-7-yl]oxymethyl]pyrazol-3-yl]pyrazolo[1,5-a]pyridin-2-yl]cyclopropanecarboxamide

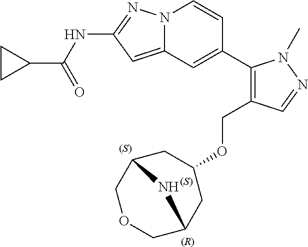

The title compound (7 mg, 2 steps, 1%) was prepared as described in Example 360 Steps A-B, except ENDO-tert-butyl (1R,5S)-7-hydroxy-3-oxa-9-azabicyclo[3.3.1]nonane-9-carboxylate was used instead of tert-butyl (R)-3-hydroxypyrrolidine-1-carboxylate in Step A and ENDO-tert-butyl (1R,5S,7s)-7-((5-bromo-1-methyl-1H-pyrazol-4-yl)methoxy)-3-oxa-9-azabicyclo[3.3.1]nonane-9-carboxylate was used instead of tert-butyl (R)-3-((5-bromo-1-methyl-1H-pyrazol-4-yl)methoxy)pyrrolidine-1-carboxylate in Step B. MS (ESI): mass calcd. for C₂₃H₂₈N₆O₃, 436.2. m/z found, 439.2 [M+H]⁺. ¹H NMR (400 MHz, Methanol-d4) δ 8.49 (dt, J=7.2, 1.0 Hz, 1H), 7.73 (dd, J=2.0, 1.0 Hz, 1H), 7.60 (s, 1H), 6.98-6.89 (m, 2H), 4.37 (s, 2H), 3.86 (s, 3H), 3.76 (p, J=6.5 Hz, 1H), 3.68 (dd, J=11.2, 2.4 Hz, 2H), 3.62-3.54 (m, 2H), 2.94 (d, J=8.7 Hz, 2H), 2.22 (ddd, J=14.4, 8.5, 6.2 Hz, 2H), 1.87 (dq, J=8.1, 4.0 Hz, 1H), 1.67 (ddd, J=14.0, 6.9, 2.7 Hz, 2H), 1.00 (dt, J=4.6, 3.1 Hz, 2H), 0.91 (dt, J=8.0, 3.2 Hz, 2H).

### Example 184: (RS)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide

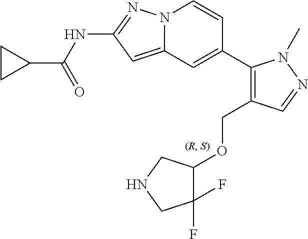 [SMILES: Cn1ncc(COC2CNCC2(F)F)c1-c1ccn2nc(NC(=O)C3CC3)cc2c1]

The title compound (170 mg, 2 steps, 28%) was prepared as described in Example 360 Steps A-B, except tert-butyl 3,3-difluoro-4-hydroxypyrrolidine-1-carboxylate was used instead of tert-butyl (R)-3-hydroxypyrrolidine-1-carboxylate in Step A. MS (ESI): mass calcd. for C₂₀H₂₂F₂N₆O₂, 416.2. m/z found, 417.2 [M+H]⁺. ¹H NMR (400 MHz, Methanol-d4) δ 8.49 (dt, J=7.2, 0.9 Hz, 1H), 7.66 (dd, J=1.9, 0.9 Hz, 1H), 7.60 (s, 1H), 6.97-6.91 (m, 1H), 6.90-6.82 (m, 1H), 4.56 (d, J=11.4 Hz, 1H), 4.45 (d, J=11.4 Hz, 1H), 3.93 (ddd, J=12.3, 9.2, 5.6 Hz, 1H), 3.86 (s, 3H), 3.25 (dd, J=12.4, 6.3 Hz, 1H), 3.21-3.00 (m, 2H), 2.82 (dd, J=12.1, 4.9 Hz, 1H), 1.87 (dt, J=8.1, 3.7 Hz, 1H), 1.00 (dt, J=4.6, 3.1 Hz, 2H), 0.96-0.83 (m, 2H).

### Example 185: N-[5-[2-methyl-4-[[(1S,5R)-9-methyl-3-oxa-9-azabicyclo[3.3.1]nonan-7-yl]oxymethyl]pyrazol-3-yl]pyrazolo[1,5-a]pyridin-2-yl]cyclopropanecarboxamide

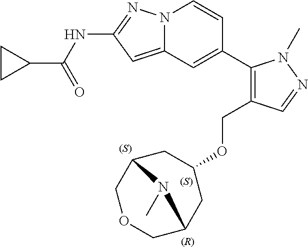

The title compound (7.7 mg, 9%) was prepared as described in Example 1 Step F, except N-[5-[2-methyl-4-[[(1S,5R)-3-oxa-9-azabicyclo[3.3.1]nonan-7-yl]oxymethyl]pyrazol-3-yl]pyrazolo[1,5-a]pyridin-2-yl]cyclopropanecarboxamide from Example 183 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₄H₃₀N₆O₃, 450.2. m/z found, 451.0 [M+H]⁺. ¹H NMR (500 MHz, Methanol-d4) δ 8.47 (dt, J=7.2, 1.0 Hz, 1H), 7.76 (dd, J=2.0, 1.0 Hz, 1H), 7.59 (s, 1H), 6.95 (dd, J=7.1, 1.8 Hz, 2H), 4.34 (s, 2H), 3.92-3.87 (m, 2H), 3.85 (s, 3H), 3.82 (dt, J=6.4, 3.2 Hz, 1H), 3.58-3.47 (m, 2H), 2.61 (d, J=6.9 Hz, 2H), 2.46 (s, 3H), 2.28 (dq, J=14.1, 7.7, 6.7 Hz, 2H), 1.86 (dq, J=8.3, 4.6, 4.1 Hz, 1H), 1.64 (d, J=14.9 Hz, 2H), 0.99 (dt, J=4.5, 3.2 Hz, 2H), 0.90 (dt, J=8.1, 3.3 Hz, 2H).

### Example 186: N-[5-[2-methyl-4-[[3-(trifluoromethyl)azetidin-3-yl]oxymethyl]pyrazol-3-yl]pyrazolo[1,5-a]pyridin-2-yl]cyclopropanecarboxamide

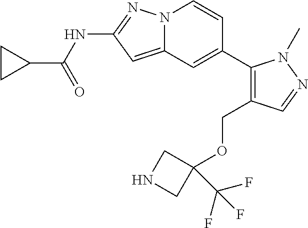 [SMILES: Cn1ncc(COC2(C(F)(F)F)CNC2)c1-c1ccn2nc(NC(=O)C3CC3)cc2c1]

The title compound (460 mg, 2 steps, 88%) was prepared as described in Example 360, Steps A-B, except tert-butyl 3-hydroxy-3-(trifluoromethyl)azetidine-1-carboxylate was used instead of tert-butyl (R)-3-hydroxypyrrolidine-1-carboxylate in Step A. MS (ESI): mass calcd. for C₂₀H₂₁F₃N₆O₂, 434.2. m/z found, 435.2 [M+H]⁺. ¹H NMR (500 MHz, Methanol-d4) δ 8.50 (dt, J=7.2, 0.9 Hz, 1H), 7.64 (dd, J=1.9, 0.9 Hz, 1H), 7.63 (s, 1H), 6.94 (s, 1H), 6.87 (dd, J=7.1, 1.9 Hz, 1H), 4.53 (s, 2H), 3.86 (s, 3H), 3.79 (d, J=10.6 Hz, 2H), 3.75-3.67 (m, 2H), 1.87 (tt, J=8.0, 4.5 Hz, 1H), 1.07-0.95 (m, 2H), 0.96-0.81 (m, 2H).

### Example 187: N-[5-[2-methyl-4-[[(2S,3R)-2-methylazetidin-3-yl]oxymethyl]pyrazol-3-yl]pyrazolo[1,5-a]pyridin-2-yl]cyclopropanecarboxamide

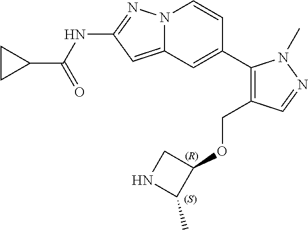

The title compound (140 mg, 2 steps, 26%) was prepared as described in Example 360, Steps A-B, except tert-butyl (2S,3R)-3-hydroxy-2-methylazetidine-1-carboxylate was used instead of tert-butyl (R)-3-hydroxypyrrolidine-1-carboxylate in Step A. MS (ESI): mass calcd. for C₂₀H₂₄N₆O₂, 380.2. m/z found, 381.2 [M+H]⁺. ¹H NMR (400 MHz, Methanol-d4) δ 8.51 (dt, J=7.1, 1.0 Hz, 1H), 7.65 (dd, J=1.9, 0.9 Hz, 1H), 7.59 (s, 1H), 7.02-6.93 (m, 1H), 6.89 (dd, J=7.2, 1.9 Hz, 1H), 4.31 (s, 2H), 3.86 (s, 4H), 3.77 (p, J=6.4 Hz, 1H), 3.42 (ddd, J=8.6, 6.7, 1.0 Hz, 1H), 1.87 (td, J=8.1, 4.1 Hz, 1H), 1.18 (d, J=6.5 Hz, 3H), 1.00 (dt, J=4.6, 3.1 Hz, 2H), 0.91 (dt, J=8.1, 3.2 Hz, 2H).

### Example 188: N-[5-[4-[[(3R,4S)-4-fluoropyrrolidin-3-yl]oxymethyl]-2-methyl-pyrazol-3-yl]pyrazolo[1,5-a]pyridin-2-yl]cyclopropanecarboxamide

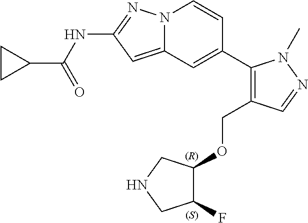

The title compound (72 mg, 2 steps, 14%) was prepared as described in Example 360, Steps A-B, except tert-butyl (3R,4S)-3-((5-bromo-1-methyl-1H-pyrazol-4-yl)methoxy)-4-fluoropyrrolidine-1-carboxylate was used instead of tert-butyl (R)-3-hydroxypyrrolidine-1-carboxylate in Step A. MS (ESI): mass calcd. for C₂₀H₂₃FN₆O₂, 398.2. m/z found, 399.2 [M+H]⁺. ¹H NMR (500 MHz, Methanol-d4) δ 8.48 (dt, J=7.2, 1.0 Hz, 1H), 7.70 (dd, J=2.0, 0.9 Hz, 1H), 7.61 (s, 1H), 6.93 (s, 1H), 6.91 (dd, J=7.2, 1.9 Hz, 1H), 5.05 (d, J=1.8 Hz, 1H), 4.48 (d, J=11.3 Hz, 1H), 4.41 (d, J=11.3 Hz, 1H), 4.04-3.91 (m, 1H), 3.87 (s, 3H), 3.15-3.02 (m, 3H), 2.85-2.74 (m, 1H), 1.86 (dd, J=8.5, 4.2 Hz, 1H), 0.99 (dt, J=4.6, 3.1 Hz, 2H), 0.90 (dt, J=8.0, 3.3 Hz, 2H).

### Example 189: (*R)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide

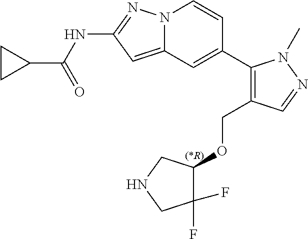

The title compound (672 mg, 80%) was isolated by separating enantiomers in Example 184. The title compound was first to elute at 3.70 min. MS (ESI): mass calcd. for C₂₀H₂₂F₂N₆O₂, 416.2. m/z found, 417.3 [M+H]⁺. ¹H NMR (400 MHz, Methanol-d4) δ 8.49 (dt, J=7.1, 1.0 Hz, 1H), 7.66 (dd, J=2.0, 0.9 Hz, 1H), 7.60 (s, 1H), 6.94 (s, 1H), 6.88 (dd, J=7.2, 1.9 Hz, 1H), 4.56 (d, J=11.4 Hz, 1H), 4.45 (d, J=11.4 Hz, 1H), 3.99-3.90 (m, 1H), 3.86 (s, 3H), 3.28-3.21 (m, 1H), 3.21-2.99 (m, 2H), 2.82 (ddd, J=12.4, 5.2, 1.6 Hz, 1H), 1.87 (tt, J=8.1, 4.7 Hz, 1H), 1.04-0.96 (m, 2H), 0.94-0.83 (m, 2H).

### Example 190: N-[5-[4-[[(2S,3R)-1,2-dimethylazetidin-3-yl]oxymethyl]-2-methyl-pyrazol-3-yl]pyrazolo[1,5-a]pyridin-2-yl]cyclopropanecarboxamide

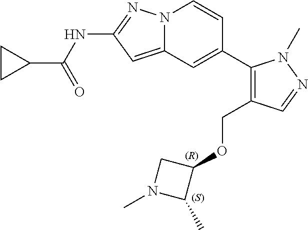

The title compound (10 mg, 15%) was prepared as described in Example 1 Step F, except N-[5-[2-methyl-4-[[(2S,3R)-2-methylazetidin-3-yl]oxymethyl]pyrazol-3-yl]pyrazolo[1,5-a]pyridin-2-yl]cyclopropanecarboxamide from Example 187 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₁H₂₆N₆O₂, 394.2. m/z found, 395.2 [M+H]⁺. ¹H NMR (500 MHz, Methanol-d4) δ 8.50 (dt, J=7.1, 0.9 Hz, 1H), 7.64 (dd, J=2.0, 1.0 Hz, 1H), 7.57 (s, 1H), 6.95 (s, 1H), 6.87 (dd, J=7.1, 1.9 Hz, 1H), 4.29 (s, 2H), 3.85 (s, 3H), 3.64 (q, J=6.3 Hz, 1H), 3.53 (ddd, J=7.2, 6.1, 0.9 Hz, 1H), 2.89 (p, J=6.3 Hz, 1H), 2.58 (t, J=7.1 Hz, 1H), 2.29 (s, 3H), 1.87 (tt, J=8.0, 4.5 Hz, 1H), 1.12 (d, J=6.3 Hz, 3H), 1.03-0.97 (m, 2H), 0.90 (dt, J=8.0, 3.3 Hz, 2H).

### Example 191: N-[5-[2-methyl-4-[(3-methylazetidin-3-yl)oxymethyl]pyrazol-3-yl]pyrazolo[1,5-a]pyridin-2-yl]cyclopropanecarboxamide

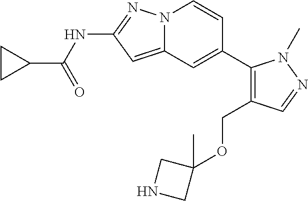 [SMILES: Cn1ncc(COC2(C)CNC2)c1-c1ccn2nc(NC(=O)C3CC3)cc2c1]

The title compound (500 mg, 2 steps, 91%) was prepared as described in Example 360, Steps A-B, except tert-butyl 3-hydroxy-3-methylazetidine-1-carboxylate was used instead of tert-butyl (R)-3-hydroxypyrrolidine-1-carboxylate in Step A. MS (ESI): mass calcd. for C₂₀H₂₄N₆O₂, 380.2. m/z found, 381.2 [M+H]⁺. ¹H NMR (400 MHz, Methanol-d4) δ 8.51 (dt, J=7.1, 0.9 Hz, 1H), 7.66 (dd, J=1.9, 0.9 Hz, 1H), 7.59 (s, 1H), 6.95 (s, 1H), 6.90 (dd, J=7.2, 1.9 Hz, 1H), 4.26 (s, 2H), 3.85 (s, 3H), 3.73-3.58 (m, 2H), 1.87 (dt, J=8.0, 3.5 Hz, 1H), 1.49 (s, 3H), 1.00 (dt, J=4.6, 3.1 Hz, 2H), 0.97-0.84 (m, 2H).

### Example 192: N-[5-[4-[(1,3-dimethylazetidin-3-yl)oxymethyl]-2-methyl-pyrazol-3-yl]pyrazolo[1,5-a]pyridin-2-yl]cyclopropanecarboxamide

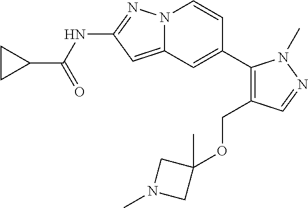 [SMILES: CN1CC(C)(OCc2cnn(C)c2-c2ccn3nc(NC(=O)C4CC4)cc3c2)C1]

The title compound (95 mg, 34%) was prepared as described in Example 1 Step F, except N-[5-[2-methyl-4-[(3-methylazetidin-3-yl)oxymethyl]pyrazol-3-yl]pyrazolo[1,5-a]pyridin-2-yl]cyclopropanecarboxamide from Example 191 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₁H₂₆N₆O₂, 394.2. m/z found, 395.2 [M+H]⁺. ¹H NMR (500 MHz, Methanol-d4) δ 8.51 (dd, J=7.1, 1.0 Hz, 1H), 7.66 (dd, J=1.9, 0.9 Hz, 1H), 7.58 (s, 1H), 6.95 (s, 1H), 6.88 (dd, J=7.2, 1.9 Hz, 1H), 4.22 (s, 2H), 3.84 (s, 3H), 3.26-3.21 (m, 2H), 3.06-3.00 (m, 2H), 2.28 (s, 3H), 1.87 (tt, J=8.1, 4.5 Hz, 1H), 1.44 (s, 3H), 0.99 (dt, J=4.5, 3.2 Hz, 2H), 0.90 (dt, J=8.1, 3.3 Hz, 2H).

### Example 193: N-[5-[4-[(3-ethylazetidin-3-yl)oxymethyl]-2-methyl-pyrazol-3-yl]pyrazolo[1,5-a]pyridin-2-yl]cyclopropanecarboxamide

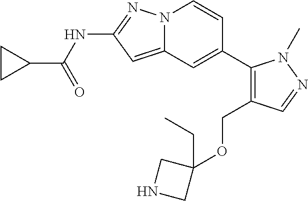 [SMILES: CCC1(OCc2cnn(C)c2-c2ccn3nc(NC(=O)C4CC4)cc3c2)CNC1]

The title compound (210 mg, 2 steps, 37%) was prepared as described in Example 360, Steps A-B, except tert-butyl 3-ethyl-3-hydroxyazetidine-1-carboxylate was used instead of tert-butyl (R)-3-hydroxypyrrolidine-1-carboxylate in Step A. MS (ESI): mass calcd. for C₂₁H₂₆N₆O₂, 394.2. m/z found, 395.2 [M+H]⁺. ¹H NMR (400 MHz, Methanol-d4) δ 8.51 (d, J=7.1 Hz, 1H), 7.67 (dd, J=2.0, 0.9 Hz, 1H), 7.60 (s, 1H), 6.94 (s, 1H), 6.90 (dd, J=7.2, 1.9 Hz, 1H), 4.22 (s, 2H), 3.85 (s, 3H), 3.64 (d, J=9.8 Hz, 2H), 3.35 (d, J=9.8 Hz, 2H), 1.88 (q, J=7.3 Hz, 3H), 1.05-0.96 (m, 2H), 0.91 (dt, J=8.0, 3.3 Hz, 2H), 0.82 (t, J=7.3 Hz, 3H).

### Example 194: (3*S,4*S)—N-[5-[2-methyl-4-[(4-methylpyrrolidin-3-yl)oxymethyl]pyrazol-3-yl]pyrazolo[1,5-a]pyridin-2-yl]cyclopropanecarboxamide

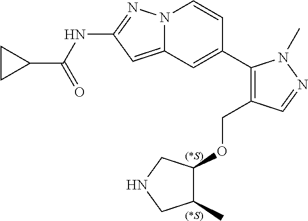

Step A. Cis-tert-butyl 3-((5-bromo-1-methyl-1H-pyrazol-4-yl)methoxy)-4-methylpyrrolidine-1-carboxylate. A mixture consisting of cis-tert-butyl 3-hydroxy-4-methylpyrrolidine-1-carboxylate (300 mg, 1.49 mmol), NaH (138 mg, 3.44 mmol) and DMA (10 mL) was stirred at 0° C. for 10 minutes. Then to the mixture was added 5-bromo-4-(chloromethyl)-1-methyl-1H-pyrazole (282 mg, 1.15 mmol) and was stirred at 25° C. for 16 h. The reaction mixture was suspended in water (10 mL), the mixture frozen using dry ice/acetone, and then lyophilized to dryness to afford the title compound (300 mg, crude) as a yellow oil.

Step B. Cis-tert-butyl 3-((5-(2-(cyclopropanecarboxamido)pyrazolo[1,5-a]pyridin-5-yl)-1-methyl-1H-pyrazol-4-yl)methoxy)-4-methylpyrrolidine-1-carboxylate. To a mixture consisting of cis-tert-butyl 3-((5-bromo-1-methyl-1H-pyrazol-4-yl)methoxy)-4-methylpyrrolidine-1-carboxylate (275 mg, 0.735 mmol), N-(5-(4,4,5,5-tetramethyl-1,3,2-dioxaborolan-2-yl)pyrazolo[1,5-a]pyridin-2-yl)cyclopropanecarboxamide (301 mg, 0.918 mmol) and K₂CO₃ (305 mg, 2.20 mmol) in 1,4-dioxane (10 mL) and H₂O (2 mL) was added [1,1′-bis(diphenylphosphino)ferrocene]dichloropalladium(II) (54 mg, 0.073 mmol) under N₂ atmosphere. The resulting mixture was stirred at 90° C. for 16 h. The reaction mixture was purified by FCC (eluent: petroleum ether:ethyl acetate=1:0 to 0:1) to afford the title compound (310 mg, 85%) as a yellow solid.

Step C. N-(5-(1-methyl-4-((((cis)-4-methylpyrrolidin-3-yl)oxy)methyl)-1H-pyrazol-5-yl)pyrazolo[1,5-a]pyridin-2-yl)cyclopropanecarboxamide. A mixture consisting of cis-tert-butyl 3-((5-(2-(cyclopropanecarboxamido)pyrazolo[1,5-a]pyridin-5-yl)-1-methyl-1H-pyrazol-4-yl)methoxy)-4-methylpyrrolidine-1-carboxylate (310 mg, 0.627 mmol) and 4M HCl/dioxane (10 mL) was stirred at room temperature. for 2 h. The reaction mixture was directly concentrated. The aqueous phase was diluted with MeOH. OH-resin was added and the resultant mixture was stirred for 0.5 h. pH showed ˜8. The insoluble resin was filtered off and the filtrate was concentrated under reduced pressure. The remaining aqueous phase was lyophilized to dryness to afford the title compound (180 mg, 61%) as a yellow solid. Brand: Alfa Aesar Ambersep 900 (OH) ion exchange resin.

Step D. (3*S,4*S)—N-[5-[2-methyl-4-[(4-methylpyrrolidin-3-yl)oxymethyl]pyrazol-3-yl]pyrazolo[1,5-a]pyridin-2-yl]cyclopropanecarboxamide. N-(5-(1-methyl-4-((((cis)-4-methylpyrrolidin-3-yl)oxy)methyl)-1H-pyrazol-5-yl)pyrazolo[1,5-a]pyridin-2-yl)cyclopropanecarboxamide (60 mg, 0.15 mmol) was purified by preparative HPLC using a Boston Prime C18 C18 150×30 mm×5 μm column (eluent: 20% to 50% (v/v) water(0.04% NH₃H₂O+10 mM NH₄HCO₃)-ACN) to afford pure product. The product was suspended in water (10 mL), the mixture frozen using dry ice/acetone, and then lyophilized to dryness to afford the title compound (30 mg, 50%) as a white solid. N-(5-(1-methyl-4-((((cis)-4-methylpyrrolidin-3-yl)oxy)methyl)-1H-pyrazol-5-yl)pyrazolo[1,5-a]pyridin-2-yl)cyclopropanecarboxamide (3 mg, 0.076 mmol) was further purified by SFC over AD 250 mm×30 mm, 10 μm (eluent: 55% to 55% (v/v) supercritical 0.1% NH₃H₂O EtOH). The title compound was second to elute at 2.59 min. The pure fractions were collected and the volatiles were removed under reduced pressure. The product was suspended in water (10 mL), the mixture frozen using dry ice/ethyl alcohol, and then lyophilized to dryness to afford the title compound (13 mg, 42%). MS (ESI): mass calcd. for C₂₁H₂₆N₆O₂, 394.2. m/z found, 395.3 [M+H]⁺. ¹H NMR (400 MHz, CD3OD) δ 8.50 (d, J=7.2 Hz, 1H), 7.68-7.62 (m, 1H), 7.60-7.56 (m, 1H), 6.96-6.90 (m, 1H), 6.89 (dd, J=1.6, 7.2 Hz, 1H), 4.49-4.38 (m, 1H), 4.32-4.21 (m, 1H), 3.91-3.87 (m, 1H), 3.86 (s, 3H), 3.09-3.00 (m, 2H), 3.00-2.93 (m, 1H), 2.64-2.55 (m, 1H), 2.21-2.06 (m, 1H), 1.95-1.79 (m, 1H), 1.03-0.94 (m, 5H), 0.93-0.87 (m, 2H)

### Example 195: (3*S,4*S)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide

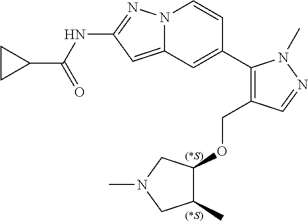

The title compound (8 mg, 4 steps, 3%) was prepared as described in Example 1 Step F, except N-(5-(1-methyl-4-((((cis)-4-methylpyrrolidin-3-yl)oxy)methyl)-1H-pyrazol-5-yl)pyrazolo[1,5-a]pyridin-2-yl)cyclopropanecarboxamide from Example 194 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. The title compound was isolated from a sample of N-(5-(4-((((cis)-1,4-dimethylpyrrolidin-3-yl)oxy)methyl)-1-methyl-1H-pyrazol-5-yl)pyrazolo[1,5-a]pyridin-2-yl)cyclopropanecarboxamide (20 mg, 0.049 mmol), which was purified by SFC over AD 250 mm×30 mm, 10 μm (eluent: 55% to 55% (v/v) supercritical 0.1% NH₃H₂O EtOH). The pure fractions were collected, and the volatiles were removed under reduced pressure. The product was suspended in water (10 mL), the mixture frozen using dry ice/ethyl alcohol, and then lyophilized to dryness to afford the title compound, second to elute, at 2.76 min. MS (ESI): mass calcd. for C₂₂H₂₈N₆O₂, 408.2. m/z found, 409.3 [M+H]⁺. ¹H NMR (400 MHz, CD3OD) δ 8.49 (d, J=6.8 Hz, 1H), 7.67 (d, J=0.4 Hz, 1H), 7.58 (s, 1H), 6.94 (s, 1H), 6.89 (dd, J=2.0, 7.2 Hz, 1H), 4.38-4.33 (m, 1H), 4.28-4.23 (m, 1H), 3.94-3.88 (m, 1H), 3.86 (s, 3H), 3.02-2.94 (m, 1H), 2.85-2.78 (m, 1H), 2.49-2.41 (m, 1H), 2.30 (s, 3H), 2.28-2.17 (m, 2H), 1.93-1.83 (m, 1H), 1.02-0.97 (m, 2H), 0.95 (d, J=6.8 Hz, 3H), 0.93-0.87 (m, 2H)

### Example 196: (3*R, 4*R)—N-[5-[2-methyl-4-[(4-methylpyrrolidin-3-yl)oxymethyl]pyrazol-3-yl]pyrazolo[1,5-a]pyridin-2-yl]cyclopropanecarboxamide

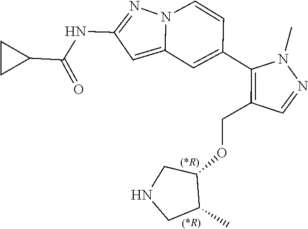 [SMILES: C[C@@H]1CNC[C@@H]1OCc1cnn(C)c1-c1ccn2nc(NC(=O)C3CC3)cc2c1]

The title compound (12 mg, 14%) was prepared as described in Example 194, except the title compound was the first to elute at 2.40 minutes. MS (ESI): mass calcd. for C₂₁H₂₆N₆O₂, 394.2. m/z found, 395.2 [M+H]⁺. ¹H NMR (400 MHz, CD3OD) δ 8.50 (d, J=7.2 Hz, 1H), 7.68-7.62 (m, 1H), 7.60-7.56 (m, 1H), 6.96-6.91 (m, 1H), 6.88 (dd, J=1.6, 7.2 Hz, 1H), 4.48-4.39 (m, 1H), 4.32-4.21 (m, 1H), 3.92-3.87 (m, 1H), 3.86 (s, 3H), 3.11-3.01 (m, 2H), 3.00-2.95 (m, 1H), 2.69-2.57 (m, 1H), 2.20-2.09 (m, 1H), 1.93-1.82 (m, 1H), 1.02-0.95 (m, 5H), 0.93-0.86 (m, 2H)

### Example 197: (3*R, 4*R)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide

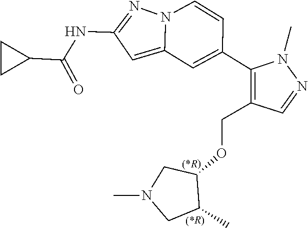 [SMILES: C[C@@H]1CN(C)C[C@@H]1OCc1cnn(C)c1-c1ccn2nc(NC(=O)C3CC3)cc2c1]

The title compound (7.5 mg, 4 steps, 3%) was prepared as described in Example 195. The title compound was first to elute at 2.35 min. MS (ESI): mass calcd. for C₂₂H₂₈N₆O₂, 408.2. m/z found, 409.3 [M+H]⁺. ¹H NMR (400 MHz, CD3OD) δ 8.50 (d, J=7.2 Hz, 1H), 7.70-7.63 (m, 1H), 7.58 (s, 1H), 6.94 (s, 1H), 6.89 (dd, J=1.6, 6.8 Hz, 1H), 4.41-4.32 (m, 1H), 4.30-4.21 (m, 1H), 3.95-3.89 (m, 1H), 3.86 (s, 3H), 3.03-2.94 (m, 1H), 2.86-2.77 (m, 1H), 2.51-2.43 (m, 1H), 2.31 (s, 3H), 2.29-2.18 (m, 2H), 1.92-1.83 (m, 1H), 1.02-0.97 (m, 2H), 0.95 (d, J=6.4 Hz, 3H), 0.93-0.87 (m, 2H)

### Example 198: 3-[[4-[2-(cyclopropanecarbonylamino)pyrazolo[1,5-a]pyridin-5-yl]-5-methyl-isoxazol-3-yl]oxymethyl]azetidine-3-carboxamide

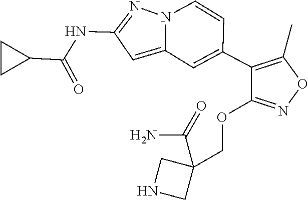 [SMILES: Cc1onc(OCC2(C(N)=O)CNC2)c1-c1ccn2nc(NC(=O)C3CC3)cc2c1]

The title compound (52 mg, 4 steps, 3%) was prepared as described in Example 5 Steps A-B then D-E, except tert-butyl 3-cyano-3-(hydroxymethyl)azetidine-1-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₀H₂₂N₆O₄, 410.2. m/z found, 411.1 [M+H]⁺. ¹H NMR (400 MHz, DMSO-d6) δ 11.04 (s, 1H), 8.50 (dd, J=7.2, 1.0 Hz, 1H), 7.67-7.60 (m, 1H), 7.41 (s, 1H), 7.16 (s, 1H), 6.90 (dd, J=7.3, 1.9 Hz, 1H), 6.85 (s, 1H), 4.64 (s, 2H), 3.73 (d, J=8.1 Hz, 2H), 3.33 (d, J=9.1 Hz, 2H), 2.54 (s, 3H), 1.98-1.85 (m, 1H), 0.89-0.73 (m, 4H).

### Example 199: N-[5-[5-methyl-3-[(3-phenylazetidin-3-yl)methoxy]isoxazol-4-yl]pyrazolo[1,5-a]pyridin-2-yl]cyclopropanecarboxamide

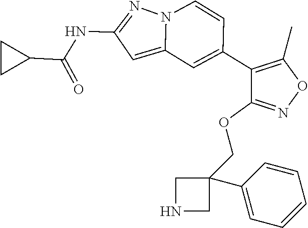 [SMILES: Cc1onc(OCC2(c3ccccc3)CNC2)c1-c1ccn2nc(NC(=O)C3CC3)cc2c1]

The title compound (30.9 mg, 3 steps, 7%) was prepared as described in Example 5 Steps A-B then D, except tert-butyl 3-(hydroxymethyl)-3-phenylazetidine-1-carboxylate was used instead of trans-rac-tert-butyl (3r,4r)-3-hydroxy-4-methoxypyrrolidine-1-carboxylate in Step A and N-(5-(3-hydroxy-5-methylisoxazol-4-yl)pyrazolo[1,5-a]pyridin-2-yl)cyclopropanecarboxamide was used instead of 1-methyl-1h-pyrazol-4-ol in Step B. MS (ESI): mass calcd. for C₂₅H₂₅N₅O₃, 443.2. m/z found, 444.2 [M+H]⁺. ¹H NMR (500 MHz, DMSO-d6) δ 11.06 (s, 1H), 8.54 (d, J=7.3 Hz, 1H), 7.47 (dd, J=2.0, 0.9 Hz, 1H), 7.36 (t, J=7.7 Hz, 2H), 7.28-7.19 (m, 3H), 6.86 (s, 1H), 6.77 (dd, J=7.2, 2.0 Hz, 1H), 4.66 (s, 2H), 3.82 (d, J=7.7 Hz, 2H), 3.63 (d, J=7.7 Hz, 2H), 2.51 (s, 3H), 1.94 (dq, J=8.0, 4.7, 3.9 Hz, 1H), 0.95-0.70 (m, 4H).

### Example 200: (rac-cis)-N-[5-[3-(4-methoxypyrrolidin-3-yl)oxy-5-methyl-isoxazol-4-yl]pyrazolo[1,5-a]pyridin-2-yl]cyclopropanecarboxamide

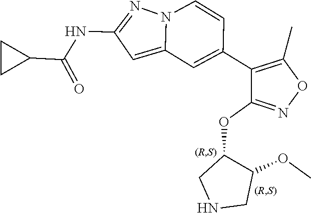

The title compound (80.3 mg, 3 steps, 7%) was prepared as described in Example 5 Steps B then D-E, except in Step B tert-butyl (3R,4R)-3-methoxy-4-((methylsulfonyl)oxy)pyrrolidine-1-carboxylate was used instead of rac-trans-tert-butyl (3r,4r)-3-methoxy-4-(tosyloxy)pyrrolidine-1-carboxylate and 4-bromo-5-methylisoxazol-3-ol was used instead of 1-methyl-1h-pyrazol-4-ol. MS (ESI): mass calcd. for C₂₀H₂₃N₅O₄, 397.2. m/z found, 398.2 [M+H]⁺. ¹H NMR (500 MHz, DMSO-d6) δ 11.04 (s, 1H), 8.60-8.51 (m, 1H), 7.72-7.64 (m, 1H), 6.96 (dd, J=7.2, 2.0 Hz, 1H), 6.83 (s, 1H), 5.09-5.01 (m, 1H), 3.96-3.87 (m, 1H), 3.27 (s, 3H), 3.24 (dd, J=12.3, 5.6 Hz, 1H), 3.08 (dd, J=11.1, 6.4 Hz, 1H), 2.89 (dd, J=12.4, 4.1 Hz, 1H), 2.73 (dd, J=11.2, 6.7 Hz, 1H), 2.53 (s, 3H), 1.98-1.86 (m, 1H), 0.90-0.70 (m, 4H).

### Example 201: N-[5-[5-methyl-3-[[(1R,4R)-5-oxa-2-azabicyclo[2.2.1]heptan-4-yl]methoxy]isoxazol-4-yl]pyrazolo[1,5-a]pyridin-2-yl]cyclopropanecarboxamide

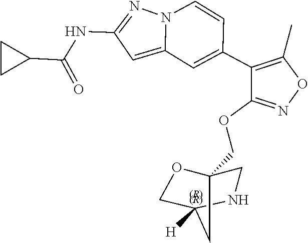

The title compound (88.2 mg, 4 steps, 19%) was prepared as described in Example 5 Steps A-B then D-E, except (1r,4r)-tert-butyl 1-(hydroxymethyl)-2-oxa-5-azabicyclo[2.2.1]heptane-5-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₁H₂₃N₅O₄, 409.2. m/z found, 410.2 [M+H]⁺. ¹H NMR (500 MHz, DMSO-d6) δ 11.06 (s, 1H), 8.61-8.55 (m, 1H), 7.69-7.48 (m, 1H), 6.90 (dd, J=7.2, 2.0 Hz, 1H), 6.84 (s, 1H), 4.65-4.48 (m, 2H), 3.81-3.72 (m, 1H), 3.68 (d, J=6.9 Hz, 1H), 3.56 (s, 1H), 2.94 (d, J=9.7 Hz, 1H), 2.88 (d, J=9.5 Hz, 1H), 2.54 (s, 3H), 2.00-1.85 (m, 1H), 1.69 (s, 2H), 0.91-0.73 (m, 4H).

### Example 202: N-(5-(3-(((3*S,4*R)-4-methoxypyrrolidin-3-yl)oxy)-5-methylisoxazol-4-yl)pyrazolo[1,5-a]pyridin-2-yl)cyclopropanecarboxamide

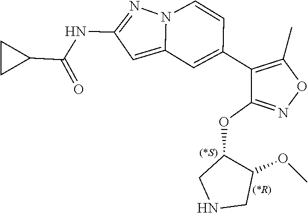

The title compound (17.5 mg, 2%) was prepared as described in Example 200, except the title compound was second to elute at 6.47 min. MS (ESI): mass calcd. for C₂₀H₂₃N₅O₄, 397.2. m/z found, 398.2 [M+H]⁺. ¹H NMR (500 MHz, DMSO-d6) δ 11.04 (s, 1H), 8.60-8.51 (m, 1H), 7.72-7.64 (m, 1H), 6.96 (dd, J=7.2, 2.0 Hz, 1H), 6.83 (s, 1H), 5.09-5.01 (m, 1H), 3.96-3.87 (m, 1H), 3.27 (s, 3H), 3.24 (dd, J=12.3, 5.6 Hz, 1H), 3.08 (dd, J=11.1, 6.4 Hz, 1H), 2.89 (dd, J=12.4, 4.1 Hz, 1H), 2.73 (dd, J=11.2, 6.7 Hz, 1H), 2.53 (s, 3H), 1.98-1.86 (m, 1H), 0.90-0.70 (m, 4H).

### Example 203: N-[5-[5-methyl-3-[[(1S,4S)-2-methyl-5-oxa-2-azabicyclo[2.2.1]heptan-4-yl]methoxy]isoxazol-4-yl]pyrazolo[1,5-a]pyridin-2-yl]cyclopropanecarboxamide

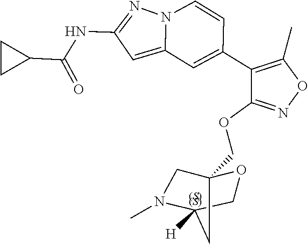

The title compound (88.2 mg, 4 steps, 19%) was prepared as described in Example 5 Steps A-B then D-E followed by Example 1 Step F, except (1 S,4S)-tert-butyl 1-(hydroxymethyl)-2-oxa-5-azabicyclo[2.2.1]heptane-5-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₂H₂₅N₅O₄, 423.2. m/z found, 424.2 [M+H]⁺. ¹H NMR (500 MHz, DMSO-d6) δ 11.06 (s, 1H), 8.64-8.56 (m, 1H), 7.63 (dd, J=2.0, 0.9 Hz, 1H), 6.90 (dd, J=7.2, 2.0 Hz, 1H), 6.84 (s, 1H), 4.58-4.48 (m, 2H), 3.97 (dd, J=7.5, 0.9 Hz, 1H), 3.64 (dd, J=7.5, 1.8 Hz, 1H), 3.38 (s, 1H), 2.93 (d, J=9.5 Hz, 1H), 2.55-2.52 (m, 3H), 2.48-2.46 (m, 1H), 2.32 (s, 3H), 1.96-1.89 (m, 1H), 1.89-1.86 (m, 1H), 1.70-1.64 (m, 1H), 0.86-0.76 (m, 4H).

### Example 204: N-[5-[3-[(2R)-2-amino-2-phenyl-ethoxy]-5-methyl-isoxazol-4-yl]pyrazolo[1,5-a]pyridin-2-yl]cyclopropanecarboxamide

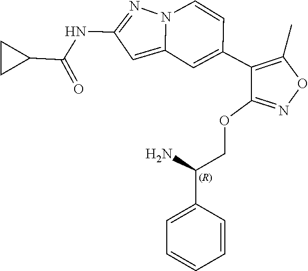

The title compound (29.5 mg, 4 steps, 21%) was prepared as described in Example 5 Steps A-B then D-E, except (R)—N-(tert-butoxycarbonyl)-2-phenylglycinol was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₃H₂₃N₅O₃, 417.2. m/z found, 418.2 [M+H]⁺. ¹H NMR (500 MHz, DMSO-d6) δ 11.04 (s, 1H), 8.55-8.42 (m, 1H), 7.49-7.43 (m, 3H), 7.34 (t, J=7.6 Hz, 2H), 7.27 (t, J=7.0 Hz, 1H), 6.83 (s, 1H), 6.79 (dd, J=7.1, 2.1 Hz, 1H), 4.39-4.18 (m, 4H), 1.96-1.89 (m, 1H), 0.88-0.76 (m, 4H).

### Example 205: (RS)—N-[5-[3-[(3-methoxypyrrolidin-3-yl)methoxy]-5-methyl-isoxazol-4-yl]pyrazolo[1,5-a]pyridin-2-yl]cyclopropanecarboxamide

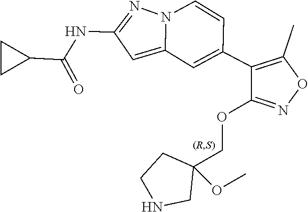

The title compound (93.4 mg, 4 steps, 13%) was prepared as described in Example 5 Steps A-B then D-E, except tert-butyl 3-(hydroxymethyl)-3-methoxypyrrolidine-1-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₁H₂₅N₅O₄, 411.2. m/z found, 412.2 [M+H]⁺. ¹H NMR (500 MHz, DMSO-d6) δ 11.06 (s, 1H), 8.59 (d, J=7.2 Hz, 1H), 7.65 (d, J=1.9 Hz, 1H), 6.92 (dd, J=6.6, 1.7 Hz, 1H), 6.84 (s, 1H), 4.42 (d, J=11.1 Hz, 1H), 4.36 (d, J=11.1 Hz, 1H), 3.18 (s, 3H), 2.91-2.76 (m, 4H), 2.54 (s, 3H), 1.97-1.89 (m, 1H), 1.89-1.80 (m, 1H), 1.75-1.64 (m, 1H), 0.86-0.77 (m, 4H).

### Example 206: N-[5-[3-[(4-methoxy-4-piperidyl)methoxy]-5-methyl-isoxazol-4-yl]pyrazolo[1,5-a]pyridin-2-yl]cyclopropanecarboxamide

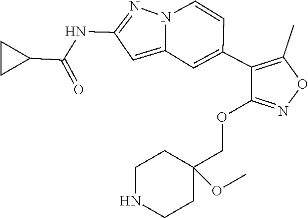 [SMILES: COC1(COc2noc(C)c2-c2ccn3nc(NC(=O)C4CC4)cc3c2)CCNCC1]

The title compound (60.7 mg, 4 steps, 8%) was prepared as described in Example 5 Steps A-B then D-E, except tert-butyl 4-(hydroxymethyl)-4-methoxypiperidine-1-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₂H₂₇N₅O₄, 425.2. m/z found, 426.2 [M+H]⁺. ¹H NMR (500 MHz, DMSO-d6) δ 11.06 (s, 1H), 8.61-8.56 (m, 1H), 7.64 (dd, J=2.0, 0.9 Hz, 1H), 6.91 (dd, J=7.2, 2.0 Hz, 1H), 6.83 (s, 1H), 4.24 (s, 2H), 3.16 (s, 3H), 2.75-2.61 (m, 4H), 2.53 (s, 3H), 1.96-1.88 (m, 1H), 1.73-1.62 (m, 2H), 1.54-1.38 (m, 2H), 0.88-0.74 (m, 4H).

### Example 207: N-[5-[3-[(2S)-2-(dimethylamino)-2-phenyl-ethoxy]-5-methyl-isoxazol-4-yl]pyrazolo[1,5-a]pyridin-2-yl]cyclopropanecarboxamide

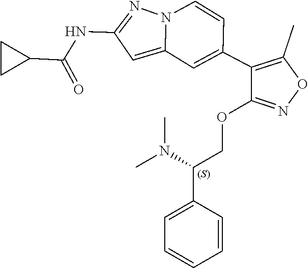

The title compound (16.6 mg, 71%) was prepared as described in Example 1 Step F, except N-[5-[3-[(2S)-2-amino-2-phenyl-ethoxy]-5-methyl-isoxazol-4-yl]pyrazolo[1,5-a]pyridin-2-yl]cyclopropanecarboxamide from Example 31 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₅H₂₇N₅O₃, 445.2. m/z found, 446.3 [M+H]⁺. ¹H NMR (500 MHz, DMSO-d6) δ 11.04 (s, 1H), 8.46 (dd, J=7.2, 1.1 Hz, 1H), 7.38 (d, J=4.3 Hz, 4H), 7.35-7.29 (m, 2H), 6.77 (s, 1H), 6.67 (dd, J=7.3, 2.0 Hz, 1H), 4.64 (dd, J=10.5, 5.8 Hz, 1H), 4.52 (dd, J=10.5, 7.0 Hz, 1H), 3.74 (dd, J=7.0, 5.8 Hz, 1H), 2.51 (s, 3H), 2.17 (s, 6H), 1.97-1.88 (m, 1H), 0.90-0.77 (m, 4H).

### Example 208: (rac-cis)-N-[5-[3-(4-fluoropyrrolidin-3-yl)oxy-5-methyl-isoxazol-4-yl]pyrazolo[1,5-a]pyridin-2-yl]cyclopropanecarboxamide

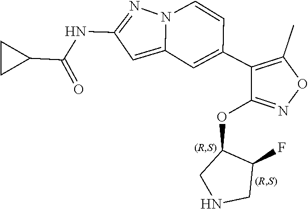

The title compound (93.3 mg, 4 steps, 7%) was prepared as described in Example 5 Steps A-B then D-E, except trans-tert-butyl 3-fluoro-4-hydroxypyrrolidine-1-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₁₉H₂₀FN₅O₃, 385.2. m/z found, 386.2 [M+H]⁺. ¹H NMR (400 MHz, DMSO-d6) δ 11.04 (s, 1H), 8.61-8.50 (m, 1H), 7.67-7.58 (m, 1H), 6.90 (dd, J=7.2, 1.9 Hz, 1H), 6.85 (s, 1H), 5.26 (d, J=55.2 Hz, 1H), 5.07-4.89 (m, 1H), 3.22-3.15 (m, 1H), 3.03-2.97 (m, 1H), 2.97-2.90 (m, 1H), 2.90-2.83 (m, 1H), 2.53 (s, 3H), 1.97-1.86 (m, 1H), 0.87-0.76 (m, 4H).

### Example 209: (*S)—N-(5-(3-((3-methoxypyrrolidin-3-yl)methoxy)-5-methylisoxazol-4-yl)pyrazolo[1,5-a]pyridin-2-yl)cyclopropanecarboxamide

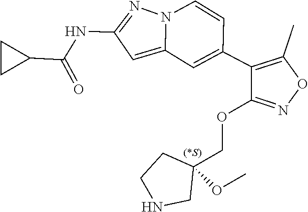

The title compound (33 mg, 4 steps, 5%) was prepared as described in Example 205. The title compound was the second to elute at 9.08 minutes. The compound was purified via SFC separation IG (2×25 cm), 50% ethanol (0.1% DEA)/CO₂, 100 bar, 50 mL/min, 220 nm, inj vol.: 1 mL, 5 mg/mL methanol:DCM. MS (ESI): mass calcd. for C₂₁H₂₅N₅O₄, 411.2. m/z found, 412.2 [M+H]⁺. ¹H NMR (500 MHz, DMSO-d6) δ 11.06 (s, 1H), 8.59 (d, J=7.2 Hz, 1H), 7.65 (d, J=1.9 Hz, 1H), 6.92 (dd, J=6.6, 1.7 Hz, 1H), 6.84 (s, 1H), 4.42 (d, J=11.1 Hz, 1H), 4.36 (d, J=11.1 Hz, 1H), 3.18 (s, 3H), 2.91-2.76 (m, 4H), 2.54 (s, 3H), 1.97-1.89 (m, 1H), 1.89-1.80 (m, 1H), 1.75-1.64 (m, 1H), 0.86-0.77 (m, 4H).

### Example 210: N-(5-(3-(((3*S,4*R)-4-fluoropyrrolidin-3-yl)oxy)-5-methylisoxazol-4-yl)pyrazolo[1,5-a]pyridin-2-yl)cyclopropanecarboxamide

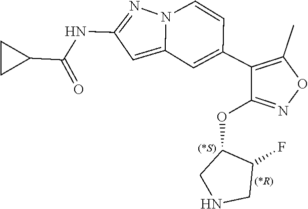

The title compound (43 mg, 4 steps, 3%) was prepared as described in Example 208. The title compound was second to elute at 6.61 min via SFC separation. The preparative method was AD-H (2×25 cm) 40% isopropanol (0.1% DEA)/CO2, 100 bar 60 mL/min, 220 nm inj vol.: 0.5 mL, 8 mg/mL isopropanol:DCM. MS (ESI): mass calcd. for C₁₉H₂₀FN₈O₃, 385.2. m/z found, 386.2 [M+H]⁺. ¹H NMR (400 MHz, DMSO-d6) δ 11.04 (s, 1H), 8.61-8.50 (m, 1H), 7.67-7.58 (m, 1H), 6.90 (dd, J=7.2, 1.9 Hz, 1H), 6.85 (s, 1H), 5.26 (d, J=55.2 Hz, 1H), 5.07-4.89 (m, 1H), 3.22-3.15 (m, 1H), 3.03-2.97 (m, 1H), 2.97-2.90 (m, 1H), 2.90-2.83 (m, 1H), 2.53 (s, 3H), 1.97-1.86 (m, 1H), 0.87-0.76 (m, 4H).

### Example 211: (rac-cis)-N-[5-[3-[(3-fluoro-4-piperidyl)oxy]-5-methyl-isoxazol-4-yl]pyrazolo[1,5-a]pyridin-2-yl]cyclopropanecarboxamide

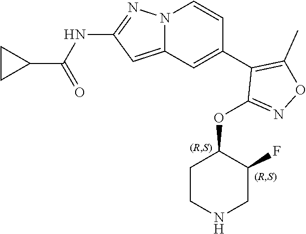 [SMILES: Cc1onc(O[C@@H]2CCNC[C@@H]2F)c1-c1ccn2nc(NC(=O)C3CC3)cc2c1]

The title compound (143 mg, 4 steps, 10%) was prepared as described in Example 5 Steps A-B then D-E, except trans-1-boc-3-fluoro-4-hydroxypiperidine was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. MS (ESI): mass calcd. for C₂₀H₂₂FN₅O₃, 399.2. m/z found, 400.2 [M+H]⁺. ¹H NMR (400 MHz, DMSO-d6) δ 11.05 (s, 1H), 8.59 (dd, J=7.3, 2.2 Hz, 1H), 7.64 (s, 1H), 6.90 (dd, J=7.2, 2.4 Hz, 1H), 6.85 (d, J=2.2 Hz, 1H), 5.10-4.89 (m, 2H), 3.25-3.12 (m, 2H), 3.03-2.85 (m, 2H), 2.76-2.65 (m, 1H), 2.53 (d, J=2.6 Hz, 3H), 2.04-1.84 (m, 3H), 0.88-0.76 (m, 4H).

### Example 212: Exo-Rac-N-[5-[3-(2-azabicyclo[2.2.1]heptan-5-yloxy)-5-methyl-isoxazol-4-yl]pyrazolo[1,5-a]pyridin-2-yl]cyclopropanecarboxamide

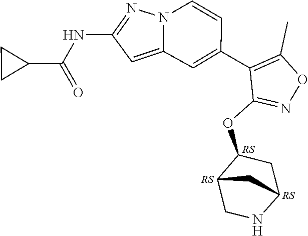 [SMILES: Cc1onc(O[C@H]2C[C@H]3C[C@@H]2CN3)c1-c1ccn2nc(NC(=O)C3CC3)cc2c1]

The title compound (253 mg, 4 steps, 16%) was prepared as described in Example 5 Steps A-B then D-E, except (1r,4r,5r)-rel-tert-butyl 5-hydroxy-2-azabicyclo[2.2.1]heptane-2-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step. MS (ESI): mass calcd. for C₂₁H₂₃N₅O₃, 393.2. m/z found, 394.2 [M+H]⁺. ¹H NMR (400 MHz, DMSO-d6) δ 11.04 (s, 1H), 8.58-8.51 (m, 1H), 7.61 (dd, J=2.1, 0.9 Hz, 1H), 6.91-6.82 (m, 2H), 4.69 (d, J=6.6 Hz, 1H), 3.41-3.35 (m, 1H), 2.74 (dd, J=10.0, 4.2 Hz, 1H), 2.67 (dd, J=3.8, 2.0 Hz, 1H), 2.51 (s, 3H), 2.40 (d, J=10.0 Hz, 1H), 2.05-1.97 (m, 1H), 1.96-1.87 (m, 1H), 1.62 (dd, J=18.4, 11.6 Hz, 2H), 1.37 (d, J=9.7 Hz, 1H), 0.87-0.77 (m, 4H).

### Example 213: N-[5-[3-[[(2R)-1-(cyanomethyl)azetidin-2-yl]methoxy]-5-methyl-isoxazol-4-yl]pyrazolo[1,5-a]pyridin-2-yl]cyclopropanecarboxamide

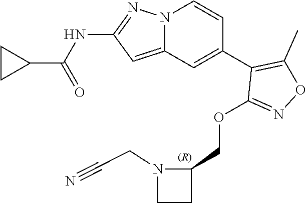

The title compound (7.2 mg, 5 steps, 2%) was prepared as described in Example 87. MS (ESI): mass calcd. for C₂₁H₂₂N₆O₃, 406.2. m/z found, 407.1 [M+H]⁺. ¹H NMR (500 MHz, DMSO-d6) δ 11.05 (s, 1H), 8.57 (dd, J=7.1, 1.0 Hz, 1H), 7.66 (dd, J=2.0, 1.0 Hz, 1H), 6.92 (dd, J=7.2, 2.0 Hz, 1H), 6.85 (s, 1H), 4.32 (dd, J=10.9, 3.8 Hz, 1H), 4.25 (dd, J=10.9, 5.8 Hz, 1H), 3.77-3.68 (m, 1H), 3.65 (s, 2H), 3.26 (dd, J=7.4, 3.1 Hz, 1H), 3.14-3.04 (m, 1H), 2.53 (s, 3H), 2.15-2.03 (m, 2H), 1.96-1.87 (m, 1H), 0.88-0.73 (m, 4H).

### Example 214: N-[5-[5-methyl-3-[[(2R)-4,6,6-trimethylmorpholin-2-yl]methoxy]isoxazol-4-yl]pyrazolo[1,5-a]pyridin-2-yl]cyclopropanecarboxamide

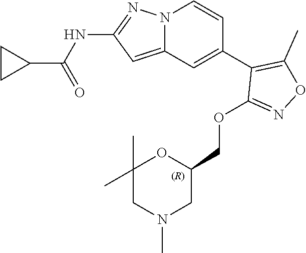

The title compound (30.7 mg, 74%) was prepared as described in Example 1 Step F, except N-[5-[3-[[(2R)-6,6-dimethylmorpholin-2-yl]methoxy]-5-methyl-isoxazol-4-yl]pyrazolo[1,5-a]pyridin-2-yl]cyclopropanecarboxamide from Example 86 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₃H₂₉N₅O₄, 439.2. m/z found, 440.3 [M+H]⁺. ¹H NMR (400 MHz, DMSO-d6) δ 11.05 (s, 1H), 8.58 (dt, J=7.2, 1.0 Hz, 1H), 7.69 (dd, J=2.0, 0.9 Hz, 1H), 6.93 (dd, J=7.2, 2.0 Hz, 1H), 6.84 (s, 1H), 4.27-4.14 (m, 2H), 4.14-4.02 (m, 1H), 2.76 (d, J=11.0 Hz, 1H), 2.53 (s, 3H), 2.15 (s, 3H), 1.97-1.85 (m, 1H), 1.74-1.64 (m, 2H), 1.27 (s, 3H), 1.12 (s, 3H), 0.88-0.72 (m, 4H).

### Example 215: N-(5-(3-(((3*S,4*R)-3-fluoropiperidin-4-yl)oxy)-5-methylisoxazol-4-yl)pyrazolo[1,5-a]pyridin-2-yl)cyclopropanecarboxamide

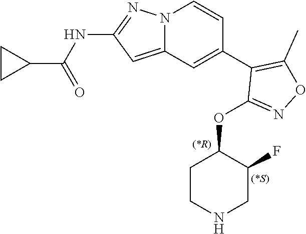 [SMILES: Cc1onc(O[C@@H]2CCNC[C@@H]2F)c1-c1ccn2nc(NC(=O)C3CC3)cc2c1]

The title compound (65 mg, 4 steps, 4%) was prepared as described in Example 211 and was isolated via SFC separation with the preparative method as follows: AD-H (2×25 cm) 50% ethanol (DEA)/CO₂, 100 bar, 50 mL/min, 220 nm inj. Vol.: 1 mg/mL methanol. The title compound was first to elute at 6.02 min. MS (ESI): mass calcd. for C₂₀H₂₂FN₅O₃, 399.2. m/z found, 400.2 [M+H]⁺. ¹H NMR (400 MHz, DMSO-d6) δ 11.05 (s, 1H), 8.59 (dd, J=7.3, 2.2 Hz, 1H), 7.64 (s, 1H), 6.90 (dd, J=7.2, 2.4 Hz, 1H), 6.85 (d, J=2.2 Hz, 1H), 5.10-4.89 (m, 2H), 3.25-3.12 (m, 2H), 3.03-2.85 (m, 2H), 2.76-2.65 (m, 1H), 2.53 (d, J=2.6 Hz, 3H), 2.04-1.84 (m, 3H), 0.88-0.76 (m, 4H).

### Example 216: N-(5-(3-(((3*R,4*S)-3-fluoropiperidin-4-yl)oxy)-5-methylisoxazol-4-yl)pyrazolo[1,5-a]pyridin-2-yl)cyclopropanecarboxamide

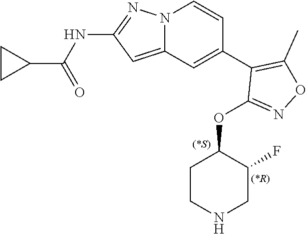 [SMILES: Cc1onc(O[C@@H]2CCNC[C@H]2F)c1-c1ccn2nc(NC(=O)C3CC3)cc2c1]

The title compound (90 mg, 4 steps, 6%) was prepared as described in Example 211 and was isolated via SFC separation with the preparative method as follows: AD-H (2×25 cm) 50% ethanol (DEA)/CO₂, 100 bar, 50 mL/min, 220 nm inj. Vol.: 1 mg/mL methanol. The title compound was second to elute at 8.85 min. MS (ESI): mass calcd. for C₂₀H₂₂FN₅O₃, 399.2. m/z found, 400.2 [M+H]⁺. ¹H NMR (400 MHz, DMSO-d6) δ 11.05 (s, 1H), 8.59 (dd, J=7.3, 2.2 Hz, 1H), 7.64 (s, 1H), 6.90 (dd, J=7.2, 2.4 Hz, 1H), 6.85 (d, J=2.2 Hz, 1H), 5.10-4.89 (m, 2H), 3.25-3.12 (m, 2H), 3.03-2.85 (m, 2H), 2.76-2.65 (m, 1H), 2.53 (d, J=2.6 Hz, 3H), 2.04-1.84 (m, 3H), 0.88-0.76 (m, 4H).

### Example 217: N-(5-(3-(((1*S,4*S,5*R)-2-azabicyclo[2.2.1]heptan-5-yl)oxy)-5-methylisoxazol-4-yl)pyrazolo[1,5-a]pyridin-2-yl)cyclopropanecarboxamide

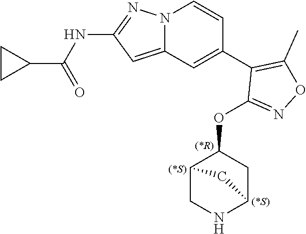 [SMILES: Cc1onc(O[C@H]2C[C@@H]3C[C@H]2CN3)c1-c1ccn2nc(NC(=O)C3CC3)cc2c1]

The title compound (92 mg, 4 steps, 6%) was prepared as described in Example 212 and was isolated via SFC separation with the preparative method as follows: AD-H (2×25 cm) 50% ethanol (DEA)/CO₂, 100 bar, 50 mL/min, 220 nm inj. Vol.: 1 mg/mL methanol. The title compound was second to elute at 10.99 min. MS (ESI): mass calcd. for C₂₁H₂₃N₅O₃, 393.2. m/z found, 394.2 [M+H]⁺. ¹H NMR (400 MHz, DMSO-d6) δ 11.04 (s, 1H), 8.58-8.51 (m, 1H), 7.61 (dd, J=2.1, 0.9 Hz, 1H), 6.91-6.82 (m, 2H), 4.69 (d, J=6.6 Hz, 1H), 3.41-3.35 (m, 1H), 2.74 (dd, J=10.0, 4.2 Hz, 1H), 2.67 (dd, J=3.8, 2.0 Hz, 1H), 2.51 (s, 3H), 2.40 (d, J=10.0 Hz, 1H), 2.05-1.97 (m, 1H), 1.96-1.87 (m, 1H), 1.62 (dd, J=18.4, 11.6 Hz, 2H), 1.37 (d, J=9.7 Hz, 1H), 0.87-0.77 (m, 4H).

### Example 218: (*R)—N-[5-[3-(9-azaspiro[3.5]nonan-6-yloxy)-5-methyl-isoxazol-4-yl]pyrazolo[1,5-a]pyridin-2-yl]cyclopropanecarboxamide

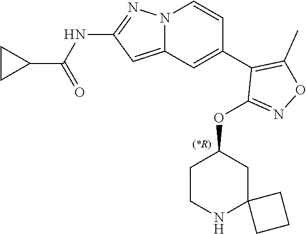 [SMILES: Cc1onc(O[C@@H]2CCNC3(CCC3)C2)c1-c1ccn2nc(NC(=O)C3CC3)cc2c1]

The title compound (20 mg, 4 steps, 4%) was prepared as described in Example 5 Steps A-B then D-E, except tert-butyl 8-hydroxy-5-azaspiro[3.5]nonane-5-carboxylate was used instead of trans-rac-tert-butyl (3r,4r)-3-hydroxy-4-methoxypyrrolidine-1-carboxylate in Step A and 4-bromo-5-methylsoxazol-3-ol was used instead of 1-methyl-1h-pyrazol-4-ol in Step B. The compound was purified after Step D via SFC purification with the method AS_EtOH_DEA_5_40_28ML_8MIN and was the second to elute at 2.784 min. MS (ESI): mass calcd. for C₂₃H₂₇N₅O₃, 421.2. m/z found, 422.3 [M+H]⁺. ¹H NMR (400 MHz, DMSO-d6) δ 11.07 (s, 1H), 8.57 (d, J=7.2 Hz, 1H), 7.63 (d, J=0.8 Hz, 1H), 6.89 (dd, J=1.6, 7.2 Hz, 1H), 6.85 (s, 1H), 4.78-4.67 (m, 1H), 2.87-2.79 (m, 1H), 2.63-2.55 (m, 1H), 2.50-2.50 (m, 3H), 2.27-2.18 (m, 1H), 2.06-1.98 (m, 1H), 1.97-1.90 (m, 2H), 1.90-1.83 (m, 2H), 1.78-1.66 (m, 3H), 1.60-1.46 (m, 2H), 0.88-0.77 (m, 4H)

### Example 219: (*R)—N-[5-[5-methyl-3-[(9-methyl-9-azaspiro[3.5]nonan-6-yl)oxy]isoxazol-4-yl]pyrazolo[1,5-a]pyridin-2-yl]cyclopropanecarboxamide

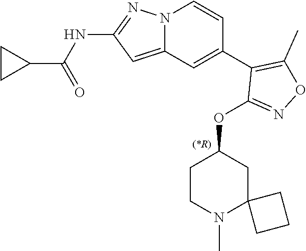 [SMILES: Cc1onc(O[C@@H]2CCN(C)C3(CCC3)C2)c1-c1ccn2nc(NC(=O)C3CC3)cc2c1]

The title compound (22.7 mg, 12%) was prepared as described in Example 1 Step F, except (*R)—N-[5-[3-(9-azaspiro[3.5]nonan-6-yloxy)-5-methyl-isoxazol-4-yl]pyrazolo[1,5-a]pyridin-2-yl]cyclopropanecarboxamide from Example 218 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₄H₂₉N₅O₃, 435.2. m/z found, 436.3 [M+H]⁺. ¹H NMR (400 MHz, DMSO-d6) δ 11.06 (s, 1H), 8.57 (d, J=7.2 Hz, 1H), 7.63 (s, 1H), 6.89 (dd, J=2.0, 7.2 Hz, 1H), 6.85 (s, 1H), 4.76-4.68 (m, 1H), 2.68-2.57 (m, 1H), 2.50-2.50 (m, 3H), 2.43-2.34 (m, 1H), 2.32-2.17 (m, 5H), 2.14-2.05 (m, 1H), 2.03-1.96 (m, 1H), 1.95-1.88 (m, 1H), 1.75-1.58 (m, 6H), 0.87-0.77 (m, 4H)

### Example 220: N-[5-[3-[(2S)-2-amino-2-(2-fluorophenyl)ethoxy]-5-methyl-isoxazol-4-yl]pyrazolo[1,5-a]pyridin-2-yl]cyclopropanecarboxamide

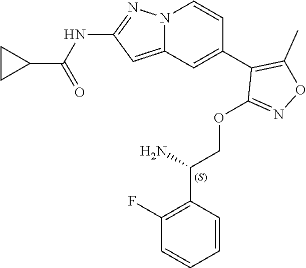

Step A. (S)-tert-butyl (1-(2-fluorophenyl)-2-hydroxyethyl)carbamate. A mixture consisting of (S)-2-amino-2-(2-fluorophenyl)ethanol (500 mg, 3.22 mmol), di-tert-butyl dicarbonate (774 mg, 3.54 mmol), TEA (652 mg, 6.45 mmol), and DCM (20 mL) was stirred at room-temperature for 1 hour under N₂ atmosphere. TLC (petroleum ether:ethyl acetate=1:1, R_{f}=0.4) showed a new spot formed. The mixture was quenched with Sat.NaHCO₃ (5 mL) and extracted with CH₂Cl₂ (30 mL×2). The organic layers were concentrated to dryness under reduced pressure to afford the crude product (800 mg, crude), which was used in the next step without further purification.

Step B. N-(5-(4,4,5,5-tetramethyl-1,3,2-dioxaborolan-2-yl)pyrazolo[1,5-a]pyridin-2-yl)cyclopropanecarboxamide. DIAD (256 mg, 1.47 mmol) was added to a solution consisting of (S)-tert-butyl (1-(2-fluorophenyl)-2-hydroxyethyl)carbamate (250 mg, 0.979 mmol), 4-bromo-5-methylisoxazol-3-ol (209 mg, 1.18 mmol), PPh₃ (514 mg, 1.96 mmol), and THE (15 mL). The resultant mixture was stirred at 70° C. for 12 hours. The reaction mixture was concentrated to dryness under reduced pressure to afford the crude product, which was purified by FCC (eluent: DCM:MeOH=1:0 to 10:1) to afford the title compound (200 mg, 49%) as a yellow solid.

Step C. (S)-tert-butyl (2-((4-(2-(cyclopropanecarboxamido)pyrazolo[1,5-a]pyridin-5-yl)-5-methylisoxazol-3-yl)oxy)-1-(2-fluorophenyl)ethyl)carbamate. A mixture consisting of (S)-tert-butyl (2-((4-bromo-5-methylisoxazol-3-yl)oxy)-1-(2-fluorophenyl)ethyl)carbamate (170 mg, 0.409 mmol), N-(5-(4,4,5,5-tetramethyl-1,3,2-dioxaborolan-2-yl)pyrazolo[1,5-a]pyridin-2-yl)cyclopropanecarboxamide (174 mg, 0.532 mmol), K₃PO₄ (261 mg, 1.23 mmol) was added to a 40 mL sealed tube and the resulting mixture dissolved in 1,4-dioxane (9 mL) and H₂O (3 mL). The mixture was sparged with N₂ for 1 minute and then treated with Pd(dppf)Cl₂ (59.9 mg, 0.082 mmol). The mixture was stirred at 80° C. for 5 hours. The reaction mixture was concentrated to dryness under reduced pressure to afford the crude product, which was purified by FCC (eluent: petroleum ether:ethyl acetate=1:0 to 0:1) to afford the title compound (130 mg, 59%) as a yellow solid.

Step D. N-[5-[3-[(2S)-2-amino-2-(2-fluorophenyl)ethoxy]-5-methyl-isoxazol-4-yl]pyrazolo[1,5-a]pyridin-2-yl]cyclopropanecarboxamide. HCl/1,4-dioxane (10 mL, 40 mmol, 4.0 M) was added to (S)-tert-butyl (2-((4-(2-(cyclopropanecarboxamido)pyrazolo[1,5-a]pyridin-5-yl)-5-methylisoxazol-3-yl)oxy)-1-(2-fluorophenyl)ethyl)carbamate (130 mg, 0.243 mmol) under stirring at room-temperature. The reaction was concentrated to dryness under reduced pressure to give the crude product, which was purified by preparative HPLC using a Boston Prime C18 150×30 mm×5 μm column (eluent: 40% to 70% (v/v) CH₃CN and H₂O with 0.04% NH₃H₂O+10 mM NH₄HCO₃) to afford pure product. The product was suspended in water (10 mL), the mixture frozen using dry ice/ethanol, and then lyophilized to dryness to afford the title compound (55.3 mg, 52%) as a white solid. MS (ESI): mass calcd. for C₂₃H₂₂FN₅O₃, 435.2. m/z found, 436.1 [M+H]⁺. ¹H NMR (400 MHz, CDCl₃) δ 8.91 (s, 1H), 8.22 (d, J=6.8 Hz, 1H), 7.57-7.44 (m, 1H), 7.33-7.25 (m, 1H), 7.23-7.15 (m, 1H), 7.12-7.03 (m, 1H), 7.00-6.90 (m, 1H), 6.68 (d, J=7.2 Hz, 1H), 4.81-4.68 (m, 1H), 4.60-4.41 (m, 2H), 2.50 (s, 3H), 1.64-1.51 (m, 1H), 1.21-1.08 (m, 2H), 0.96-0.84 (m, 2H) 19F NMR (376 MHz, CDCl3) δ −118.41-−118.66 (m, 1F)

### Example 221: N-[5-[3-[(2R)-2-amino-2-(2-fluorophenyl)ethoxy]-5-methyl-isoxazol-4-yl]pyrazolo[1,5-a]pyridin-2-yl]cyclopropanecarboxamide

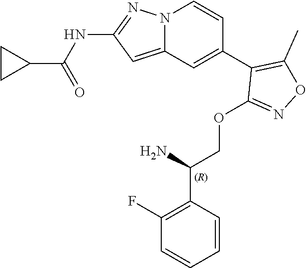

The title compound (20.8 mg, 4 steps, 3%) was prepared as described in Example 220, except (R)-2-amino-2-(2-fluorophenyl)ethanol was used instead of (S)-2-amino-2-(2-fluorophenyl)ethanol in Step A, (R)-tert-butyl (1-(2-fluorophenyl)-2-hydroxyethyl)carbamate was used instead of (S)-tert-butyl (1-(2-fluorophenyl)-2-hydroxyethyl)carbamate in Step B, (R)-tert-butyl (2-((4-bromo-5-methylisoxazol-3-yl)oxy)-1-(2-fluorophenyl)ethyl)carbamate was used instead of (S)-tert-butyl (2-((4-bromo-5-methylisoxazol-3-yl)oxy)-1-(2-fluorophenyl)ethyl)carbamate in Step C, and (R)-tert-butyl (2-((4-(2-(cyclopropanecarboxamido)pyrazolo[1,5-a]pyridin-5-yl)-5-methylisoxazol-3-yl)oxy)-1-(2-fluorophenyl)ethyl)carbamate was used instead (S)-tert-butyl (2-((4-(2-(cyclopropanecarboxamido)pyrazolo[1,5-a]pyridin-5-yl)-5-methylisoxazol-3-yl)oxy)-1-(2-fluorophenyl)ethyl)carbamate in Step D. MS (ESI): mass calcd. for C₂₃H₂₂FN₅O₃, 435.2. m/z found, 436.1 [M+H]⁺. ¹H NMR (400 MHz, CD3OD) δ 8.31 (d, J=7.6 Hz, 1H), 7.59-7.49 (m, 1H), 7.42-7.29 (m, 2H), 7.26-7.18 (m, 1H), 7.16-7.06 (m, 1H), 6.82 (s, 1H), 6.76 (dd, J=2.0, 7.2 Hz, 1H), 4.65 (t, J=6.4 Hz, 1H), 4.53-4.47 (m, 1H), 4.46-4.40 (m, 1H), 2.49 (s, 3H), 1.94-1.81 (m, 1H), 1.06-0.97 (m, 2H), 0.95-0.86 (m, 2H) 19F NMR (376 MHz, CD3OD) δ−120.17-−121.03 (m, 1F)

### Example 222: N-[5-[3-[(2R)-2-(dimethylamino)-2-(2-fluorophenyl)ethoxy]-5-methyl-isoxazol-4-yl]pyrazolo[1,5-a]pyridin-2-yl]cyclopropanecarboxamide

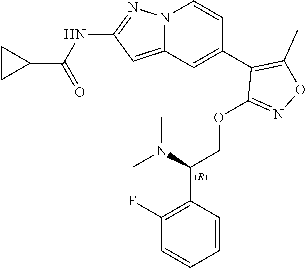

The title compound (35.6 mg, 36%) was prepared as described in Example 1 Step F, except N-[5-[3-[(2R)-2-amino-2-(2-fluorophenyl)ethoxy]-5-methyl-isoxazol-4-yl]pyrazolo[1,5-a]pyridin-2-yl]cyclopropanecarboxamide from Example 221 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₅H₂₆FN₅O₃, 463.2. m/z found, 464.1 [M+H]⁺. ¹H NMR (400 MHz, CD3OD) δ 8.25 (d, J=6.8 Hz, 1H), 7.54-7.46 (m, 1H), 7.43-7.34 (m, 1H), 7.27-7.12 (m, 3H), 6.75 (s, 1H), 6.63 (dd, J=2.0, 7.6 Hz, 1H), 4.80-4.73 (m, 1H), 4.65-4.55 (m, 1H), 4.27-4.19 (m, 1H), 2.48 (s, 3H), 2.28 (s, 6H), 1.93-1.83 (m, 1H), 1.06-0.97 (m, 2H), 0.95-0.86 (m, 2H) 19F NMR (376 MHz, CD3OD) δ −118.01-−119.20 (m, 1F)

### Example 223: N-[5-[3-[(2S)-2-amino-2-(2-pyridyl)ethoxy]-5-methyl-isoxazol-4-yl]pyrazolo[1,5-a]pyridin-2-yl]cyclopropanecarboxamide

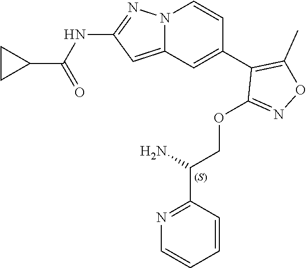

The title compound (13.8 mg, 4 steps, 3%) was prepared as described in Example 220, except (S)-2-amino-2-(pyridin-2-yl)ethanol hydrochloride was used instead of (S)-2-amino-2-(2-fluorophenyl)ethanol in Step A, (S)-tert-butyl (2-hydroxy-1-(pyridin-2-yl)ethyl)carbamate was used instead of (S)-tert-butyl (1-(2-fluorophenyl)-2-hydroxyethyl)carbamate in Step B, (S)-tert-butyl (2-((4-bromo-5-methylisoxazol-3-yl)oxy)-1-(pyridin-2-yl)ethyl)carbamate was used instead of (S)-tert-butyl (2-((4-bromo-5-methylisoxazol-3-yl)oxy)-1-(2-fluorophenyl)ethyl)carbamate in Step C, and (S)-tert-butyl (2-((4-(2-(cyclopropanecarboxamido)pyrazolo[1,5-a]pyridin-5-yl)-5-methylisoxazol-3-yl)oxy)-1-(pyridin-2-yl)ethyl)carbamate was used instead (S)-tert-butyl (2-((4-(2-(cyclopropanecarboxamido)pyrazolo[1,5-a]pyridin-5-yl)-5-methylisoxazol-3-yl)oxy)-1-(2-fluorophenyl)ethyl)carbamate in Step D. MS (ESI): mass calcd. for C₂₂H₂₂N₆O₃, 418.2. m/z found, 419.1 [M+H]⁺. ¹H NMR (400 MHz, CD3OD) δ 8.57 (s, 1H), 8.33 (d, J=7.2 Hz, 1H), 7.89-7.78 (m, 1H), 7.53 (d, J=7.2 Hz, 1H), 7.41-7.28 (m, 2H), 6.81 (s, 1H), 6.75 (dd, J=1.6, 7.2 Hz, 1H), 4.61-4.53 (m, 3H), 2.49 (s, 3H), 1.95-1.81 (m, 1H), 1.06-0.98 (m, 2H), 0.94-0.87 (m, 2H)

### Example 224: N-[5-[5-methyl-3-[(2S)-2-(methylamino)-2-(2-pyridyl)ethoxy]isoxazol-4-yl]pyrazolo[1,5-a]pyridin-2-yl]cyclopropanecarboxamide

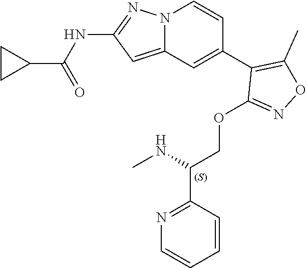

The title compound (16.1 mg, 11%) was prepared as described in Example 1 Step F, except (S)—N-(5-(3-(2-amino-2-(pyridin-2-yl)ethoxy)-5-methylisoxazol-4-yl)pyrazolo[1,5-a]pyridin-2-yl)cyclopropanecarboxamide hydrochloride from Example 223 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₃H₂₄N₆O₃, 432.2. m/z found, 433.1 [M+H]⁺. ¹H NMR (400 MHz, DMSO-d6) δ 11.06 (s, 1H), 8.62-8.55 (m, 1H), 8.49 (d, J=7.6 Hz, 1H), 7.86-7.76 (m, 1H), 7.53 (d, J=7.6 Hz, 1H), 7.42 (s, 1H), 7.36-7.28 (m, 1H), 6.81 (s, 1H), 6.72 (dd, J=1.6, 7.2 Hz, 1H), 4.53-4.42 (m, 2H), 4.11-4.03 (m, 1H), 2.53-2.52 (m, 3H), 2.24 (s, 3H), 2.04-1.88 (m, 2H), 0.86-0.80 (m, 4H)

### Example 225: N-[5-[3-[(2S)-2-amino-2-phenyl-propoxy]-5-methyl-isoxazol-4-yl]pyrazolo[1,5-a]pyridin-2-yl]cyclopropanecarboxamide

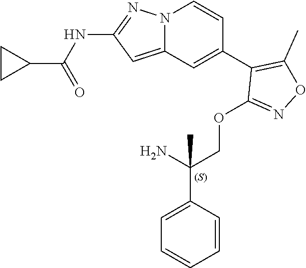

Step A. (4S)-tert-butyl 4-methyl-4-phenyl-1,2,3-oxathiazolidine-3-carboxylate 2-oxide. A solution of SOCl₂ (1.18 g, 9.95 mmol) and CH₃CN (20 mL) was cooled to −40° C., prior to dropwise addition of (S)-tert-butyl (1-hydroxy-2-phenylpropan-2-yl)carbamate (1.0 g, 4.0 mmol) and CH₃CN (10 mL). The internal temperature was maintained at −40° C. during the addition. Pyridine (1.57 g, 19.9 mmol) was then added, and the reaction mixture was stirred slowly warm to room temperature for 5 hours. The reaction was concentrated to dryness under reduced pressure to afford a yellow residue, which was purified by FCC (eluent: petroleum ether:ethyl acetate=1:0 to 1:1) to afford the title compound (600 mg, 51%) as a yellow oil.

Step B. (S)-tert-butyl 4-methyl-4-phenyl-1,2,3-oxathiazolidine-3-carboxylate 2,2-dioxide. A mixture consisting of (4S)-tert-butyl 4-methyl-4-phenyl-1,2,3-oxathiazolidine-3-carboxylate 2-oxide (300 mg, 1.01 mmol), NaIO₄ (259 mg, 1.21 mmol), RuCl₃ (23 mg, 0.11 mmol) MeCN (5 mL), and H₂O (5 mL). The mixture was stirred at 0° C. for 3 h. The reaction mixture was poured into ice water (3 mL) and ethyl acetate (50 mL), and the layers were separated. The organics were dried over anhydrous Na₂SO₄, concentrated to dryness under reduced pressure to afford the crude product, which was purified by FCC (eluent: petroleum ether:ethyl acetate=1:0 to 2:1) to afford the title compound (240 mg, 76%) as a white solid.

Step C. (S)-tert-butyl (1-((4-bromo-5-methylisoxazol-3-yl)oxy)-2-phenylpropan-2-yl)carbamate (S)-tert-butyl 4-methyl-4-phenyl-1,2,3-oxathiazolidine-3-carboxylate 2,2-dioxide (400 mg, 1.28 mmol), 4-bromo-5-methylisoxazol-3-ol (454 mg, 2.55 mmol), Cs₂CO₃ (1.25 g, 3.83 mmol), and CH₃CN (10 mL) were added to a 40 mL microwave tube. The resultant mixture was stirred while heating at 120° C. via microwave irradiation for 1 h. The reaction mixture was filtered, the filter was concentrated to dryness under reduced pressure to give the crude product, which was purified by preparative HPLC with a Phenomenex Gemini-NX 150×30 mm×5 μm column (eluent: 55% to 85% (v/v) CH₃CN and aqueous HCl (0.006 N)) to afford pure product. The product was suspended in water (10 mL), the mixture frozen using dry ice/ethanol, and then lyophilized to dryness to afford the title compound (130 mg, 25%) as a yellow oil.

Step D. (S)-tert-butyl (1-((4-(2-(cyclopropanecarboxamido)pyrazolo[1,5-a]pyridin-5-yl)-5-methylisoxazol-3-yl)oxy)-2-phenylpropan-2-yl)carbamate. A mixture consisting of (S)-tert-butyl (1-((4-bromo-5-methylisoxazol-3-yl)oxy)-2-phenylpropan-2-yl)carbamate (110 mg, 0.267 mmol), N-(5-(4,4,5,5-tetramethyl-1,3,2-dioxaborolan-2-yl)pyrazolo[1,5-a]pyridin-2-yl)cyclopropanecarboxamide (175 mg, 0.535 mmol), K₃PO₄ (109 mg, 0.802 mmol) was added to a 8 mL sealed tube and the resulting mixture dissolved in 1,4-dioxane (3 mL) and H₂O (1 mL). The mixture was sparged with N₂ for 1 minute and then treated with Pd(dppf)Cl₂ (39 mg, 0.053 mmol). The mixture was stirred at 80° C. for 3 hours. The reaction mixture was filtered through a pad of Celite® and the pad washed with ethyl acetate (10 mL). The filtrate was concentrated to dryness under reduced pressure to afford the crude product, which was purified by FCC (eluent: petroleum ether:ethyl acetate=1:0 to 0:1) to afford the product (60 mg, 38%) as a yellow solid.

Step E. (4S)-tert-butyl 4-methyl-4-phenyl-1,2,3-oxathiazolidine-3-carboxylate 2-oxide. HCl/1,4-dioxane (5 mL, 20 mmol, 4 M) was added to a solution consisting of (S)-tert-butyl (1-((4-(2-(cyclopropanecarboxamido)pyrazolo[1,5-a]pyridin-5-yl)-5-methylisoxazol-3-yl)oxy)-2-phenylpropan-2-yl)carbamate (80 mg, 0.15 mmol), and 1,4-dioxane (5 mL). The reaction mixture was stirred at room-temperature for 2 h. The reaction mixture was concentrated to dryness under reduced pressure to afford the title compound (80 mg, crude) as a brown solid. 30 mg of the material was purified by preparative HPLC with a Phenomenex Gemini-NX 150×30 mm×5 μm column (eluent: 10% to 40% (v/v) CH₃CN and aqueous HCl (0.006 N)) to afford pure product. The product was suspended in water (10 mL), the mixture frozen using dry ice/ethanol, and then lyophilized to dryness to afford the title compound (14.1 mg, 19%) as a brown solid. MS (ESI): mass calcd. for C₂₄H₂₅N₅O₃, 431.2. m/z found, 432.2 [M+H]⁺. ¹H NMR (400 MHz, CD3OD) δ 8.32 (d, J=7.2 Hz, 1H), 7.62-7.45 (m, 5H), 7.29 (s, 1H), 6.69 (dd, J=1.6, 7.2 Hz, 1H), 4.80-4.64 (m, 2H), 2.51 (s, 3H), 1.95-1.80 (m, 4H), 1.07-0.98 (m, 2H), 0.97-0.85 (m, 2H)

### Example 226: N-[5-[3-[(2S)-2-(dimethylamino)-2-phenyl-propoxy]-5-methyl-isoxazol-4-yl]pyrazolo[1,5-a]pyridin-2-yl]cyclopropanecarboxamide

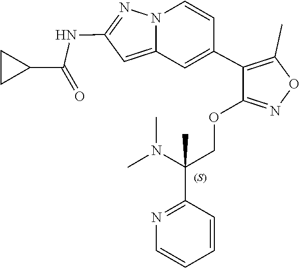

The title compound (5.2 mg, 10%) was prepared as described in Example 1 Step F, except N-[5-[3-[(2S)-2-amino-2-phenyl-propoxy]-5-methyl-isoxazol-4-yl]pyrazolo[1,5-a]pyridin-2 yl]cyclopropanecarboxamide from Example 226 was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₆H₂₉N₅O₃, 459.2. m/z found, 460.2 [M+H]⁺. ¹H NMR (400 MHz, CD3OD) δ 8.26 (d, J=7.2 Hz, 1H), 7.61-7.54 (m, 2H), 7.44-7.35 (m, 2H), 7.34-7.28 (m, 1H), 7.23 (s, 1H), 6.79 (s, 1H), 6.61 (dd, J=1.6, 7.2 Hz, 1H), 4.58-4.44 (m, 2H), 2.48 (s, 3H), 2.22 (s, 6H), 1.93-1.83 (m, 1H), 1.50 (s, 3H), 1.06-0.98 (m, 2H), 0.95-0.86 (m, 2H)

### Example 227: (3R, 3aS, 7aR)—N-[5-[3-(1,2,3,3a,5,6,7,7a-octahydropyrano[3,2-b]pyrrol-3-yloxy)-5-methyl-isoxazol-4-yl]pyrazolo[1,5-a]pyridin-2-yl]cyclopropanecarboxamide

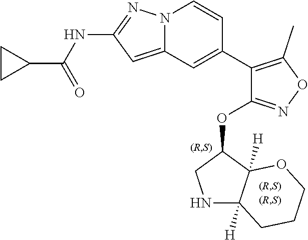 [SMILES: Cc1onc(O[C@@H]2CN[C@@H]3CCCO[C@@H]32)c1-c1ccn2nc(NC(=O)C3CC3)cc2c1]

Step A. Tert-butyl (3R,3aS,7aR)-3-((4-bromo-5-methylisoxazol-3-yl)oxy)hexahydropyrano[3,2-b]pyrrole-1(2H)-carboxylate. The title compound (373 mg, 66%) was prepared as described in Example 3 Step A, except tert-butyl (3R,3aS,7aR)-3-((4-bromo-5-methylisoxazol-3-yl)oxy)hexahydropyrano[3,2-b]pyrrole-1(2H)-carboxylate was used instead of tert-butyl (S)-2-(hydroxymethyl)-3,3-dimethylazetidine-1-carboxylate and 4-bromo-5-methylisoxazol-3-ol was used instead of Intermediate 4.

Step B. (3R, 3aS, 7aR)—N-[5-[3-(1,2,3,3a,5,6,7,7a-octahydropyrano[3,2-b]pyrrol-3-yloxy)-5-methyl-isoxazol-4-yl]pyrazolo[1,5-a]pyridin-2-yl]cyclopropanecarboxamide. The title compound (5.5 mg, 1%) was prepared as described in Example 5 Steps D-E, except tert-butyl (3R,3aS,7aR)-3-((4-bromo-5-methylisoxazol-3-yl)oxy)hexahydropyrano[3,2-b]pyrrole-1(2H)-carboxylate was used in stead of rac-cis-tert-butyl (3s,4r)-3-((5-bromo-1-methyl-1H-pyrazol-4-yl)oxy)-4-methoxypyrrolidine-1-carboxylate in Step D and tert-butyl (3R,3aS,7aR)-3-((4-(2-(cyclopropanecarboxamido)pyrazolo[1,5-a]pyridin-5-yl)-5-methylisoxazol-3-yl)oxy)hexahydropyrano[3,2-b]pyrrole-1(2H)-carboxylate was used instead of rac-cis-tert-butyl (3s,4r)-3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate in Step E. MS (ESI): mass calcd. for C₂₂H₂₅N₅O₄, 423.2. m/z found, 424.2 [M+H]⁺. ¹H NMR (500 MHz, DMSO-d6) δ 11.03 (s, 1H), 8.56 (dt, J=7.2, 0.9 Hz, 1H), 7.64 (dd, J=2.0, 0.9 Hz, 1H), 6.92 (dd, J=7.2, 2.0 Hz, 1H), 6.83 (s, 1H), 5.05 (td, J=8.1, 4.1 Hz, 1H), 4.03 (dd, J=4.1, 2.4 Hz, 1H), 3.83-3.75 (m, 1H), 3.28-3.21 (m, 2H), 3.08-3.03 (m, 2H), 2.51 (s, 3H), 1.96-1.87 (m, 1H), 1.87-1.77 (m, 1H), 1.72-1.63 (m, 2H), 1.26 (d, J=13.4 Hz, 1H), 0.85-0.77 (m, 4H).

### Example 228: N-(5-(5-methyl-3-(((3*S,3a*R,7a*S)-octahydropyrano[3,2-b]pyrrol-3-yl)oxy)isoxazol-4-yl)pyrazolo[1,5-a]pyridin-2-yl)cyclopropanecarboxamide

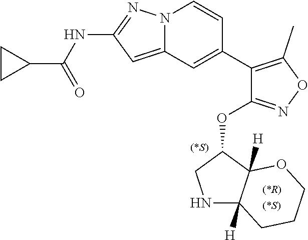 [SMILES: Cc1onc(O[C@H]2CN[C@H]3CCCO[C@H]32)c1-c1ccn2nc(NC(=O)C3CC3)cc2c1]

The title compound (44 mg, 3 steps, 7%) was prepared as described in Example 227. The title compound was isolated via SFC separation with the following preparative method: AD-H (2×25 cm), 65% isopropanol (0.2% DEA)/CO₂, 100 bar, 60 mL/min, 220 nm, inj vol.: 1 mL, 8 mg/mL ethanol. The title compound was second to elute at 3.95 min. MS (ESI): mass calcd. for C₂₂H₂₅N₅O₄, 423.2. m/z found, 424.2 [M+H]⁺. ¹H NMR (500 MHz, DMSO-d6) δ 11.03 (s, 1H), 8.56 (dt, J=7.2, 0.9 Hz, 1H), 7.64 (dd, J=2.0, 0.9 Hz, 1H), 6.92 (dd, J=7.2, 2.0 Hz, 1H), 6.83 (s, 1H), 5.05 (td, J=8.1, 4.1 Hz, 1H), 4.03 (dd, J=4.1, 2.4 Hz, 1H), 3.83-3.75 (m, 1H), 3.28-3.21 (m, 2H), 3.08-3.03 (m, 2H), 2.51 (s, 3H), 1.96-1.87 (m, 1H), 1.87-1.77 (m, 1H), 1.72-1.63 (m, 2H), 1.26 (d, J=13.4 Hz, 1H), 0.85-0.77 (m, 4H).

### Example 229: N-[5-[2-methyl-4-[[(3R)-pyrrolidin-3-yl]methoxy]pyrazol-3-yl]pyrazolo[1,5-a]pyridin-2-yl]cyclopropanecarboxamide

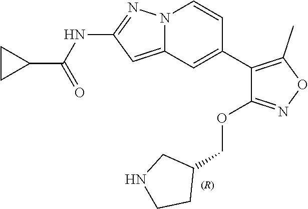

Step A. tert-butyl (R)-3-(((5-(2-(cyclopropanecarboxamido)pyrazolo[1,5-a]pyridin-5-yl)-1-methyl-1H-pyrazol-4-yl)oxy)methyl)pyrrolidine-1-carboxylate. A mixture consisting of N-(5-(4-hydroxy-1-methyl-1H-pyrazol-5-yl)pyrazolo[1,5-a]pyridin-2-yl)cyclopropanecarboxamide (300 mg, 1.01 mmol), (R)-tert-butyl 3-(bromomethyl)pyrrolidine-1-carboxylate (320 mg, 1.21 mmol), Cs₂CO₃ (822 mg, 2.52 mmol), and DMF (20 mL) was stirred overnight at room temperature. The reaction mixture was concentrated under reduced pressure to give the crude product which was purified by FCC (eluent: petroleum ether:ethyl acetate=1:0 to 0:1) to afford product (240 mg, 43%) as a light yellow solid.

Step B. N-[5-[2-methyl-4-[[(3R)-pyrrolidin-3-yl]methoxy]pyrazol-3-yl]pyrazolo[1,5-a]pyridin-2-yl]cyclopropanecarboxamide. A solution consisting of (R)-tert-butyl 3-(((5-(2-(cyclopropanecarboxamido)pyrazolo[1,5-a]pyridin-5-yl)-1-methyl-1H-pyrazol-4-yl)oxy)methyl)pyrrolidine-1-carboxylate (100 mg, 0.208 mmol), HCl/EtOAc (5 mL) was stirred at room temperature. for 2 h. The reaction mixture was concentrated under reduced pressure to give a crude product which was purified by preparative HPLC with Xtimate C18 150×25 mm×10 um, column (eluent: 15% to 45% (v/v) CH₃CN and H₂O with 0.04% NH₃H₂O+10 mM NH₄HCO₃) to afford product.

The product was suspended in water (10 mL), the mixture frozen using dry ice/EtOH, and then lyophilized to dryness to afford P1 (20.3 mg, 26%) as a white solid. MS (ESI): mass calcd. for C₂₀H₂₄N₆O₂, 380.2. m/z found, 381.2 [M+H]⁺. ¹H NMR (400 MHz, DMSO-d6) δ 11.10 (s, 1H), 8.63-8.57 (m, 1H), 7.68 (d, J=0.8 Hz, 1H), 7.48-7.43 (m, 1H), 6.95 (dd, J=1.6, 6.8 Hz, 1H), 6.90 (s, 1H), 3.94-3.80 (m, 5H), 3.23-3.06 (m, 1H), 2.92-2.71 (m, 2H), 2.64-2.54 (m, 1H), 2.45-2.35 (m, 1H), 2.00-1.74 (m, 2H), 1.71-1.33 (m, 1H), 0.87-0.78 (m, 4H)

### Example 230: N-[5-[2-methyl-4-[[(2S)-morpholin-2-yl]methoxy]pyrazol-3-yl]pyrazolo[1,5-a]pyridin-2-yl]cyclopropanecarboxamide

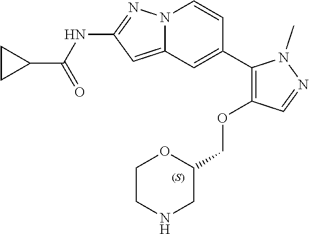

The title compound (20.6 mg, 3 steps, 2%) was prepared as described in Example 5 Steps A-B then E, except (S)-tert-butyl 2-(hydroxymethyl)morpholine-4-carboxylate was used instead of trans-rac-tert-butyl (3r,4r)-3-hydroxy-4-methoxypyrrolidine-1-carboxylate in Step A and Intermediate 4 was used instead of 1-methyl-1h-pyrazol-4-ol in Step B. MS (ESI): mass calcd. for C₂₀H₂₄N₆O₃, 396.2. m/z found, 397.2 [M+H]⁺. ¹H NMR (400 MHz, DMSO-d6) δ 11.10 (s, 1H), 8.60 (d, J=7.2 Hz, 1H), 7.70 (d, J=0.8 Hz, 1H), 7.45 (s, 1H), 6.96 (dd, J=1.6, 7.2 Hz, 1H), 6.89 (s, 1H), 3.93-3.86 (m, 2H), 3.85 (s, 3H), 3.73-3.67 (m, 1H), 3.64-3.57 (m, 1H), 3.43-3.39 (m, 1H), 2.82-2.75 (m, 1H), 2.67-2.55 (m, 2H), 2.47-2.39 (m, 1H), 1.98-1.88 (m, 1H), 0.87-0.79 (m, 4H)

### Example 231: N-[5-[4-[(1-aminocyclobutyl)methoxy]-2-methyl-pyrazol-3-yl]pyrazolo[1,5-a]pyridin-2-yl]cyclopropanecarboxamide

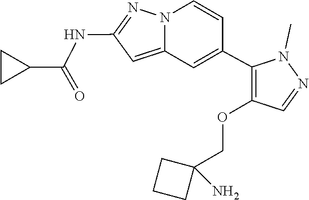 [SMILES: Cn1ncc(OCC2(N)CCC2)c1-c1ccn2nc(NC(=O)C3CC3)cc2c1]

The title compound (88.2 mg, 4 steps, 19%) was prepared as described in Example 5 Steps A-B then E, except tert-butyl (1-(hydroxymethyl)cyclobutyl)carbamate was used instead of trans-rac-tert-butyl (3r,4r)-3-hydroxy-4-methoxypyrrolidine-1-carboxylate in Step A. MS (ESI): mass calcd. for C₂₀H₂₄N₆O₂, 380.2. m/z found, 381.1 [M+H]⁺. ¹H NMR (400 MHz, DMSO-d6) δ 11.10 (s, 1H), 8.60 (d, J=7.2 Hz, 1H), 7.73 (d, J=1.2 Hz, 1H), 7.47 (s, 1H), 6.99 (dd, J=2.0, 7.2 Hz, 1H), 6.88 (s, 1H), 3.87 (s, 3H), 3.82 (s, 2H), 2.04-1.95 (m, 2H), 1.95-1.88 (m, 1H), 1.83-1.73 (m, 2H), 1.73-1.53 (m, 2H), 0.91-0.77 (m, 4H)

### Example 232: (rac-cis)-N-[5-[4-(4-methoxypyrrolidin-3-yl)oxy-2-methyl-pyrazol-3-yl]pyrazolo[1,5-a]pyridin-2-yl]cyclopropanecarboxamide

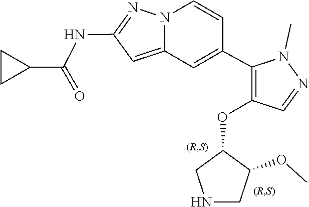 [SMILES: CO[C@@H]1CNC[C@@H]1Oc1cnn(C)c1-c1ccn2nc(NC(=O)C3CC3)cc2c1]

The title compound (29.6 mg, 4 steps, 8%) was prepared as described in Example 5 Steps A-B then E followed by Example 1 Step F, except tert-butyl 3-(hydroxymethyl)-3-methylazetidine-1-carboxylate was used instead of trans-rac-tert-butyl (3r,4r)-3-hydroxy-4-methoxypyrrolidine-1-carboxylate in Step A. MS (ESI): mass calcd. for C₂₀H₂₄N₆O₃, 396.2. m/z found, 397.2 [M+H]⁺.

¹H NMR (400 MHz, Methanol-d4) δ 8.48-8.41 (m, 1H), 7.73-7.68 (m, 1H), 7.47 (s, 1H), 7.01 (dd, J=7.2, 1.9 Hz, 1H), 6.91 (s, 1H), 4.66-4.55 (m, 1H), 3.97-3.92 (m, 1H), 3.88 (s, 3H), 3.33 (s, 3H), 3.22-3.03 (m, 3H), 2.99-2.93 (m, 1H), 1.91-1.82 (m, 1H), 1.04-0.96 (m, 2H), 0.93-0.87 (m, 2H).

### Example 233: N-[5-[2-methyl-4-[(3S,5R)-5-methylpyrrolidin-3-yl]oxy-pyrazol-3-yl]pyrazolo[1,5-a]pyridin-2-yl]cyclopropanecarboxamide

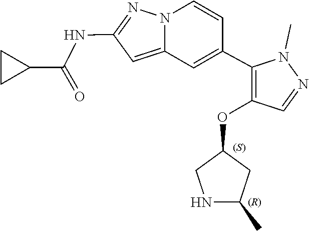

The title compound (428.3 mg, 5 steps, 58%) was prepared as described in Example 1 Steps A-E, except (2r,4r)-n-boc-4-hydroxy-2-methylpyrrolidine was used instead of (2s,4r)-tert-butyl 4-hydroxy-2-methylpyrrolidine-1-carboxylate in Step A. MS (ESI): mass calcd. for C₂₀H₂₄N₆O₂, 380.2. m/z found, 381.2 [M+H]⁺. ¹H NMR (500 MHz, Methanol-d4) δ 8.47-8.43 (m, 1H), 7.65-7.61 (m, 1H), 7.37 (s, 1H), 6.95 (dd, J=7.2, 1.9 Hz, 1H), 6.91 (s, 1H), 4.72-4.67 (m, 1H), 3.87 (s, 3H), 3.17 (d, J=12.8 Hz, 1H), 3.14-3.07 (m, 1H), 2.89 (dd, J=12.8, 5.1 Hz, 1H), 2.40-2.29 (m, 1H), 1.91-1.83 (m, 1H), 1.53-1.46 (m, 1H), 1.17 (d, J=6.5 Hz, 3H), 1.02-0.97 (m, 2H), 0.92-0.87 (m, 2H).

### Example 234: N-[5-[2-methyl-4-[[(1R,4R)-5-oxa-2-azabicyclo[2.2.1]heptan-4-yl]methoxy]pyrazol-3-yl]pyrazolo[1,5-a]pyridin-2-yl]cyclopropanecarboxamide

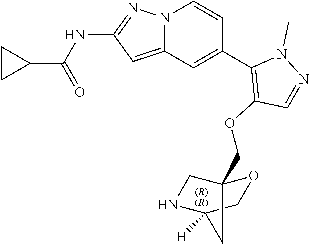

The title compound (19.8 mg, 3 steps, 8%) was prepared as described in Example 1 Steps A-B then Step E, except (1r,4r)-tert-butyl 1-(hydroxymethyl)-2-oxa-5-azabicyclo[2.2.1]heptane-5-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₁H₂₄N₆O₃, 408.2. m/z found, 409.2 [M+H]⁺. ¹H NMR (500 MHz, Methanol-d4) δ 8.47-8.43 (m, 1H), 7.67-7.63 (m, 1H), 7.47 (s, 1H), 6.97 (dd, J=7.2, 1.9 Hz, 1H), 6.91 (s, 1H), 4.34-4.26 (m, 2H), 3.90-3.86 (m, 4H), 3.83 (d, J=7.6, 0.9 Hz, 1H), 3.68-3.65 (m, 1H), 3.00-2.93 (m, 2H), 1.91-1.84 (m, 1H), 1.83-1.80 (m, 1H), 1.78-1.74 (m, 1H), 1.02-0.97 (m, 2H), 0.93-0.87 (m, 2H).

### Example 235: N-[5-[4-[(2S)-2-amino-2-phenyl-ethoxy]-2-methyl-pyrazol-3-yl]pyrazolo[1,5-a]pyridin-2-yl]cyclopropanecarboxamide

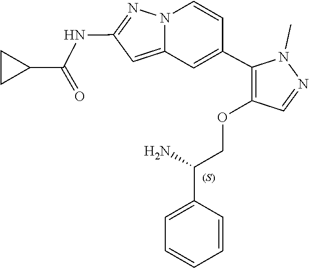

The title compound (8.8 mg, 3 steps, 1%) was prepared as described in Example 1 Steps A-B then Step E, except (S)-tert-butyl (2-hydroxy-1-phenylethyl)carbamate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₃H₂₄N₆O₂, 416.2. m/z found, 417.2 [M+H]⁺. ¹H NMR (400 MHz, DMSO-d6) δ 11.10 (s, 1H), 8.53 (d, J=7.2 Hz, 1H), 7.60 (s, 1H), 7.46-7.37 (m, 3H), 7.33-7.27 (m, 2H), 7.26-7.19 (m, 1H), 6.91-6.82 (m, 2H), 4.20-4.12 (m, 1H), 3.99-3.90 (m, 2H), 3.84 (s, 3H), 2.12-1.85 (m, 3H), 0.89-0.77 (m, 4H)

### Example 236: N-[5-[2-methyl-4-[(3S)-pyrrolidin-3-yl]oxy-pyrazol-3-yl]pyrazolo[1,5-a]pyridin-2-yl]cyclopropanecarboxamide

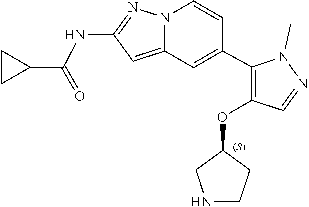

The title compound (71.1 mg, 3 steps, 22%) was prepared as described in Example 1 Steps A-B then Step E, except (R)-tert-butyl 3-hydroxypyrrolidine-1-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₁₉H₂₂N₆O₂, 366.2. m/z found, 367.2 [M+H]⁺. ¹H NMR (400 MHz, CD3OD) δ8.47-8.40 (m, 1H), 7.64-7.56 (m, 1H), 7.43-7.40 (m, 1H), 6.96-6.88 (m, 2H), 4.78-4.70 (m, 1H), 3.87 (s, 3H), 3.64-3.34 (m, 1H), 3.19-2.92 (m, 3H), 2.06-1.94 (m, 2H), 1.91-1.83 (m, 1H), 1.03-0.85 (m, 4H)

### Example 237: N-[5-[4-[(2R)-2-amino-2-phenyl-ethoxy]-2-methyl-pyrazol-3-yl]pyrazolo[1,5-a]pyridin-2-yl]cyclopropanecarboxamide

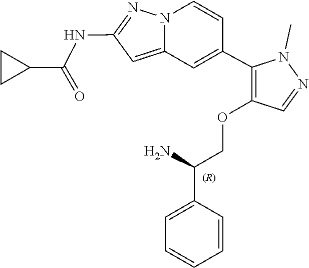

The title compound (23.5 mg, 3 steps, 4%) was prepared as described in Example 1 Steps A-B then Step E, except (R)-tert-butyl (2-hydroxy-1-phenylethyl)carbamate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₃H₂₄N₆O₂, 416.2. m/z found, 417.2 [M+H]⁺. ¹H NMR (400 MHz, DMSO-d6) δ 11.09 (s, 1H), 8.53 (d, J=7.6 Hz, 1H), 7.60 (s, 1H), 7.44 (s, 1H), 7.45-7.38 (m, 2H), 7.33-7.26 (m, 2H), 7.26-7.21 (m, 1H), 6.92-6.82 (m, 2H), 4.15 (t, J=6.4 Hz, 1H), 3.99-3.90 (m, 2H), 3.84 (s, 3H), 2.12 (br.s., 2H), 1.98-1.90 (m, 1H), 0.89-0.81 (m, 4H)

### Example 238: (3*R, 4*R)—N-[5-[2-methyl-4-(4-methylpyrrolidin-3-yl)oxy-pyrazol-3-yl]pyrazolo[1,5-a]pyridin-2-yl]cyclopropanecarboxamide

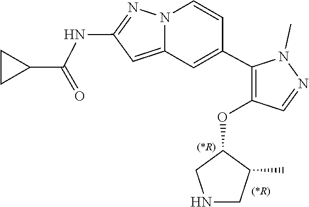

The title compound (52.9 mg, 4 steps, 26%) was prepared as described in Example 1 Steps A-B then Step E-F, except (R)-tert-butyl 2-(hydroxymethyl)pyrrolidine-1-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₀H₂₄N₆O₂, 380.2. m/z found, 381.2 [M+H]⁺. ¹H NMR (400 MHz, CD3OD) δ8.51-8.41 (m, 1H), 7.69-7.55 (m, 1H), 7.44-7.38 (m, 1H), 6.97-6.88 (m, 2H), 4.57-4.49 (m, 1H), 3.97-3.84 (m, 3H), 3.69-3.37; 3.27-3.16 (m, 3H), 3.10-3.02; 2.80-2.70 (m, 1H), 2.44-2.26 (m, 1H), 1.94-1.80 (m, 1H), 1.15-1.06 (m, 3H), 1.03-0.96 (m, 2H), 0.94-0.85 (m, 2H)

### Example 239: N-[5-[2-methyl-4-[[(2R)-1-methylpyrrolidin-2-yl]methoxy]pyrazol-3-yl]pyrazolo[1,5-a]pyridin-2-yl]cyclopropanecarboxamide

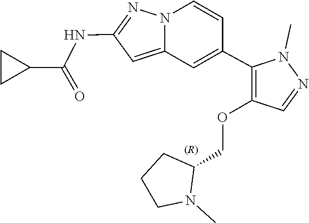

The title compound (52.9 mg, 4 steps, 26%) was prepared as described in Example 1 Steps A-B then Step E-F, except (R)-tert-butyl 2-(hydroxymethyl)pyrrolidine-1-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₁H₂₆N₆O₂, 394.2. m/z found, 395.1 [M+H]⁺. ¹H NMR (400 MHz, DMSO-d6) δ 11.13 (s, 1H), 8.61 (d, J=6.8 Hz, 1H), 7.70 (d, J=0.8 Hz, 1H), 7.47 (s, 1H), 6.97 (dd, J=2.0, 7.2 Hz, 1H), 6.88 (s, 1H), 3.96-3.90 (m, 1H), 3.86 (s, 3H), 3.84-3.78 (m, 1H), 2.94-2.87 (m, 1H), 2.49-2.44 (m, 1H), 2.28 (s, 3H), 2.19-2.09 (m, 1H), 1.97-1.82 (m, 2H), 1.68-1.58 (m, 2H), 1.58-1.47 (m, 1H), 0.88-0.79 (m, 4H)

### Example 240: (RS)—N-[5-[4-(5-azaspiro[2.3]hexan-6-ylmethoxy)-2-methyl-pyrazol-3-yl]pyrazolo[1,5-a]pyridin-2-yl]cyclopropanecarboxamide

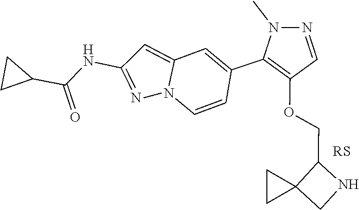 [SMILES: Cn1ncc(OCC2NCC23CC3)c1-c1ccn2nc(NC(=O)C3CC3)cc2c1]

The title compound (246 mg, 2 steps, 69%) was prepared as described in Example 3, except tert-butyl 4-(hydroxymethyl)-5-azaspiro[2.3]hexane-5-carboxylate was used instead of tert-butyl (S)-2-(hydroxymethyl)-3,3-dimethylazetidine-1-carboxylate in Step A. MS (ESI): mass calcd. for C₂₁H₂₄N₆O₂, 392.2. m/z found, 393.2 [M+H]⁺. ¹H NMR (500 MHz, DMSO-d6) δ 11.07 (s, 1H), 8.58 (dt, J=7.2, 1.0 Hz, 1H), 7.66 (dd, J=2.0, 0.9 Hz, 1H), 7.46 (s, 1H), 6.93 (dd, J=7.2, 2.0 Hz, 1H), 6.88 (s, 1H), 4.03 (t, J=5.8 Hz, 1H), 3.98-3.88 (m, 2H), 3.85 (s, 3H), 3.59 (d, J=7.4 Hz, 1H), 3.34 (d, J=7.4 Hz, 1H), 1.93 (m, J=7.7, 3.7 Hz, 1H), 0.88-0.75 (m, 4H), 0.68-0.61 (m, 1H), 0.51-0.44 (m, 1H), 0.42-0.36 (m, 1H), 0.32-0.25 (m, 1H).

### Example 241: N-[5-[4-[[(2R)-1-(2-fluoroethyl)azetidin-2-yl]methoxy]-2-methyl-pyrazol-3-yl]pyrazolo[1,5-a]pyridin-2-yl]cyclopropanecarboxamide

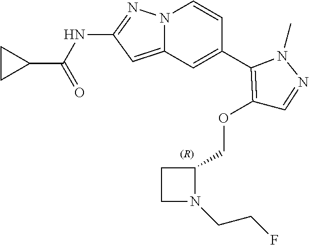

The title compound (52.9 mg, 4 steps, 26%) was prepared as described in Example 1 Steps A-B then E followed by Example 56 Step C except (R)-tert-butyl 2-(hydroxymethyl)azetidine-1-carboxylate was used instead of (2s,4r)-tert-butyl 4-hydroxy-2-methylpyrrolidine-1-carboxylate in Example 1 Step A, Intermediate 4 was used instead of 1-methyl-1h-pyrazol-4-ol in Example 1 Step B, and 1-bromo-2-fluoroethane was used instead of acetyl chloride in Example 56 Step C. MS (ESI): mass calcd. for C₂₁H₂₅FN₆O₂, 412.2. m/z found, 413.4 [M+H]⁺. ¹H NMR (400 MHz, CDCl₃) δ 8.63 (br. s., 1H), 8.30 (d, J=7.2 Hz, 1H), 7.49 (s, 1H), 7.34 (s, 1H), 7.01 (s, 1H), 6.84 (d, J=6.8 Hz, 1H), 4.52-4.40 (m, 1H), 4.40-4.28 (m, 1H), 4.06-3.93 (m, 2H), 3.90 (s, 3H), 3.56-3.45 (m, 2H), 3.00-2.79 (m, 2H), 2.77-2.63 (m, 1H), 2.11-1.99 (m, 2H), 1.64-1.54 (m, 1H), 1.21-1.10 (m, 2H), 0.96-0.87 (m, 2H) 19F NMR (376 MHz, CDCl₃) δ −221.43-−221.93 (m, 1F)

### Example 242: N-[5-[4-[(3R,4S)-4-fluoropyrrolidin-3-yl]oxy-2-methyl-pyrazol-3-yl]pyrazolo[1,5-a]pyridin-2-yl]cyclopropanecarboxamide

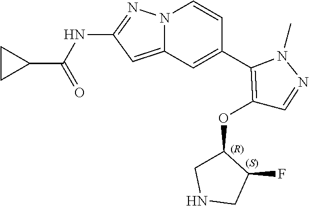

The title compound (15.5 mg, 3 steps, 2%) was prepared as described in Example 1 Steps A-B then Step E, except (3S,4S)-tert-butyl 3-fluoro-4-hydroxypyrrolidine-1-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₁₉H₂₁FN₆O₂, 384.2. m/z found, 385.1 [M+H]⁺. ¹H NMR (400 MHz, DMSO-d6) δ 11.14 (s, 1H), 9.74 (br. s., 1H), 9.39 (br. s., 1H), 8.61 (d, J=7.2 Hz, 1H), 7.72 (d, J=0.4 Hz, 1H), 7.61 (s, 1H), 6.96 (dd, J=1.6, 7.2 Hz, 1H), 6.92 (s, 1H), 5.55-5.35 (m, 1H), 4.99-4.84 (m, 1H), 3.86 (s, 3H), 3.55-3.40 (m, 3H), 3.30-3.16 (m, 1H), 1.98-1.89 (m, 1H), 0.88-0.82 (m, 4H)

### Example 243: N-[5-[4-[[(2R)-1-(2-methoxyethyl)azetidin-2-yl]methoxy]-2-methyl-pyrazol-3-yl]pyrazolo[1,5-a]pyridin-2-yl]cyclopropanecarboxamide

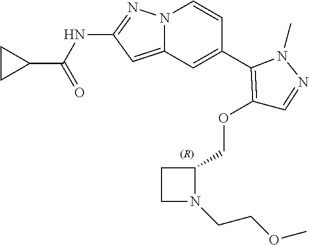

The title compound (32.5 mg, 4 steps, 36%) was prepared as described in Example 1 Steps A-B then Step E followed by Example 56 Step C, except (R)-tert-butyl 2-(hydroxymethyl)azetidine-1-carboxylate was used instead of (2s,4r)-tert-butyl 4-hydroxy-2-methylpyrrolidine-1-carboxylate in Example 1 Step A, Intermediate 4 was used instead of 1-methyl-1h-pyrazol-4-ol in Example 1 Step B, and 1-bromo-2-methoxyethane was used instead of acetyl chloride in Example 56 Step C. MS (ESI): mass calcd. for C₂₂H₂₈N₆O₃, 424.2. m/z found, 425.3 [M+H]⁺. ¹H NMR (400 MHz, CDCl₃) δ 8.48 (s, 1H), 8.29 (d, J=7.2 Hz, 1H), 7.48 (d, J=0.8 Hz, 1H), 7.34 (s, 1H), 7.00 (s, 1H), 6.85 (dd, J=2.0, 7.2 Hz, 1H), 4.06-4.00 (m, 1H), 3.99-3.92 (m, 1H), 3.90 (s, 3H), 3.48-3.39 (m, 2H), 3.36-3.31 (m, 2H), 3.26 (s, 3H), 2.93-2.84 (m, 1H), 2.84-2.76 (m, 1H), 2.59-2.51 (m, 1H), 2.09-1.97 (m, 2H), 1.63-1.54 (m, 1H), 1.18-1.12 (m, 2H), 0.94-0.88 (m, 2H)

### Example 244: N-[5-[4-[[(2R)-1-isobutylazetidin-2-yl]methoxy]-2-methyl-pyrazol-3-yl]pyrazolo[1,5-a]pyridin-2-yl]cyclopropanecarboxamide

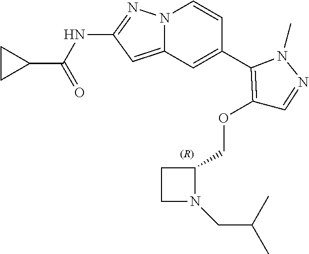

The title compound (21.5 mg, 23%) was prepared as described in Example 1 Step F, except isobutyraldehyde and (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) were used instead of paraformaldehyde and N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₃H₃₀N₆O₂, 422.2. m/z found, 423.3 [M+H]⁺. ¹H NMR (400 MHz, CDCl₃) δ 8.35 (s, 1H), 8.29 (d, J=7.6 Hz, 1H), 7.48 (s, 1H), 7.33 (s, 1H), 7.00 (s, 1H), 6.86 (dd, J=1.6, 6.8 Hz, 1H), 4.01-3.91 (m, 2H), 3.90 (s, 3H), 3.42-3.28 (m, 2H), 2.81-2.72 (m, 1H), 2.39-2.32 (m, 1H), 2.16-2.09 (m, 1H), 2.05-1.90 (m, 2H), 1.62-1.50 (m, 2H), 1.18-1.12 (m, 2H), 0.95-0.88 (m, 2H), 0.83-0.74 (m, 6H)

### Example 245: N-[5-[4-[[(2R)-1-(2-hydroxy-2-methyl-propyl)azetidin-2-yl]methoxy]-2-methyl-pyrazol-3-yl]pyrazolo[1,5-a]pyridin-2-yl]cyclopropanecarboxamide

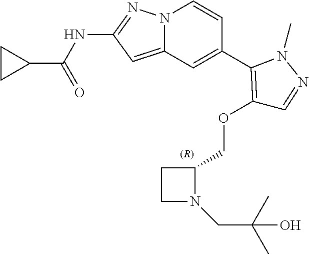

The title compound (16.5 mg, 23%) was prepared as described in Example 56 Step C, except 2,2-dimethyloxirane was used instead of acetyl chloride. MS (ESI): mass calcd. for C₂₃H₃₀N₆O₃, 438.2. m/z found, 439.3 [M+H]⁺. ¹H NMR (400 MHz, CDCl₃) δ 8.38 (s, 1H), 8.30 (d, J=7.2 Hz, 1H), 7.48 (s, 1H), 7.32 (s, 1H), 7.00 (s, 1H), 6.88 (dd, J=1.2, 7.2 Hz, 1H), 4.00-3.91 (m, 2H), 3.89 (s, 3H), 3.64-3.51 (m, 2H), 3.06-2.97 (m, 1H), 2.55-2.36 (m, 2H), 2.15-1.96 (m, 2H), 1.62-1.53 (m, 1H), 1.18-1.11 (m, 2H), 1.05 (s, 6H), 0.95-0.86 (m, 2H)

### Example 246: N-[5-[2-methyl-4-[[(2R)-1-propylazetidin-2-yl]methoxy]pyrazol-3-yl]pyrazolo[1,5-a]pyridin-2-yl]cyclopropanecarboxamide

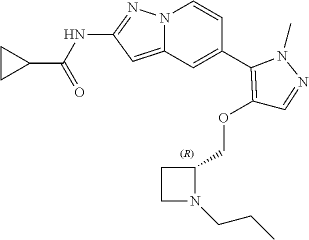

The title compound (8.6 mg, 12%) was prepared as described in Example 1 Step F, except propionaldehyde and (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) were used instead of paraformaldehyde and N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₂H₂₈N₆O₂, 408.2. m/z found, 409.1 [M+H]⁺. ¹H NMR (400 MHz, CDCl₃) δ 8.34-8.22 (m, 2H), 7.47 (s, 1H), 7.35 (s, 1H), 7.00 (s, 1H), 6.84 (dd, J=1.6, 7.2 Hz, 1H), 4.24-4.10 (m, 1H), 4.05-3.95 (m, 1H), 3.89 (s, 3H), 3.76-3.43 (m, 2H), 3.14-2.89 (m, 1H), 2.77-2.60 (m, 1H), 2.54-2.34 (m, 1H), 2.26-2.04 (m, 2H), 1.62-1.53 (m, 1H), 1.46-1.34 (m, 2H), 1.19-1.11 (m, 2H), 0.96-0.87 (m, 2H), 0.80 (t, J=7.2 Hz, 3H)

### Example 247: N-[5-[4-[[(2R)-1-(cyclopropylmethyl)azetidin-2-yl]methoxy]-2-methyl-pyrazol-3-yl]pyrazolo[1,5-a]pyridin-2-yl]cyclopropanecarboxamide

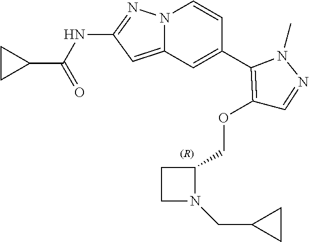

The title compound (47.8 mg, 54%) was prepared as described in Example 1 Step F, except cyclopropanecarbaldehyde and (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) were used instead of paraformaldehyde and N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₃H₂₈N₆O₂, 420.2. m/z found, 421.1 [M+H]⁺. ¹H NMR (400 MHz, CDCl₃) δ 8.48-8.35 (m, 1H), 8.29 (d, J=7.2 Hz, 1H), 7.48 (s, 1H), 7.34 (s, 1H), 7.00 (s, 1H), 6.88-6.80 (m, 1H), 4.09-3.94 (m, 2H), 3.90 (s, 3H), 3.53-3.33 (m, 2H), 2.91-2.79 (m, 1H), 2.54-2.45 (m, 1H), 2.23-2.12 (m, 1H), 2.07-1.96 (m, 2H), 1.63-1.53 (m, 1H), 1.20-1.09 (m, 2H), 0.97-0.86 (m, 2H), 0.83-0.67 (m, 1H), 0.47-0.33 (m, 2H), 0.11-0.01 (m, 2H)

### Example 248: N-[5-[2-methyl-4-[[(2S)-1-(trideuteriomethyl)azetidin-2-yl]methoxy]pyrazol-3-yl]pyrazolo[1,5-a]pyridin-2-yl]cyclopropanecarboxamide

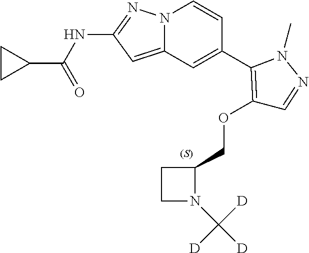

The title compound (21.9 mg, 2 steps, 18%) was prepared as described in Example 1 Step F then Step E, except D(CDO)ₙ, sodium cyanoborodeutride, and (R)—N-(5-(4-(azetidin-2-ylmethoxy)-1-methyl-1H-pyrazol-5-yl)pyrazolo[1,5-a]pyridin-2-yl)cyclopropanecarboxamide (Example 346) were used instead of paraformaldehyde, N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide, and sodium cyanoborohydride in Step F. MS (ESI): mass calcd. for C₂₀H₂₁D₃N₆O₂, 383.2. m/z found, 384.1 [M+H]⁺. ¹H NMR (400 MHz, CDCl₃) δ 8.45 (s, 1H), 8.29 (d, J=7.2 Hz, 1H), 7.49 (d, J=0.4 Hz, 1H), 7.34 (s, 1H), 6.99 (s, 1H), 6.85 (dd, J=1.6, 7.2 Hz, 1H), 4.00-3.95 (m, 2H), 3.90 (s, 3H), 3.45-3.38 (m, 1H), 3.37-3.27 (m, 1H), 2.87-2.78 (m, 1H), 2.05-1.95 (m, 2H), 1.62-1.52 (m, 1H), 1.18-1.11 (m, 2H), 0.95-0.86 (m, 2H)

### Example 249: N-[5-[2-methyl-4-[[(2R)-1-(trideuteriomethyl)pyrrolidin-2-yl]methoxy]pyrazol-3-yl]pyrazolo[1,5-a]pyridin-2-yl]cyclopropanecarboxamide

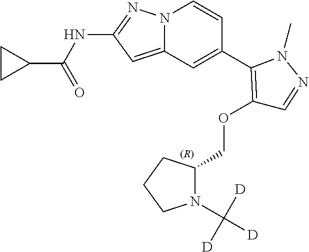

The title compound (35.8 mg, 4 steps, 11.9%) was prepared as described in Example 8 using (R)-tert-butyl 2-(hydroxymethyl)pyrrolidine-1-carboxylate in step A instead of 1-boc-3-(bromomethyl)azetidine and D(CDO)n instead of H(CHO)n in step B. MS (ESI): mass calcd. for C₂₁H₂₃D₃N₆O₂, 397.2. m/z found, 398.1 [M+H]⁺. ¹H NMR (400 MHz, DMSO-d6) δ 11.08 (s, 1H), 8.60 (d, J=7.2 Hz, 1H), 7.69 (s, 1H), 7.46 (s, 1H), 6.96 (dd, J=1.6, 7.2 Hz, 1H), 6.88 (s, 1H), 3.97-3.89 (m, 1H), 3.85 (s, 3H), 3.83-3.77 (m, 1H), 2.94-2.86 (m, 1H), 2.48-2.43 (m, 1H), 2.19-2.08 (m, 1H), 1.97-1.81 (m, 2H), 1.68-1.58 (m, 2H), 1.58-1.48 (m, 1H), 0.89-0.77 (m, 4H)

### Example 250: N-[5-[2-methyl-4-[[(2R)-1-(trideuteriomethyl)azetidin-2-yl]methoxy]pyrazol-3-yl]pyrazolo[1,5-a]pyridin-2-yl]cyclopropanecarboxamide

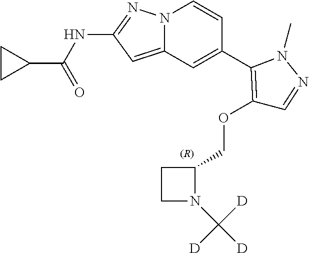

The title compound (12.3 mg, 4 steps, 11.9%) was prepared as described in Example 8 using (R)-tert-butyl 2-(hydroxymethyl)pyrrolidine-1-carboxylate in step A instead of 1-boc-3-(bromomethyl)azetidine and D(CDO)n instead of H(CHO)n in step B. MS (ESI): mass calcd. for C₂₀H₂₁D₃N₆O₂, 383.2. m/z found, 384.1 [M+H]⁺. ¹H NMR (400 MHz, CDCl₃) δ 8.33 (br. s., 1H), 8.28 (d, J=7.2 Hz, 1H), 7.49 (d, J=0.4 Hz, 1H), 7.33 (s, 1H), 6.99 (s, 1H), 6.85 (dd, J=2.0, 7.2 Hz, 1H), 3.98-3.94 (m, 2H), 3.90 (s, 3H), 3.44-3.37 (m, 1H), 3.36-3.26 (m, 1H), 2.86-2.77 (m, 1H), 2.04-1.93 (m, 2H), 1.61-1.53 (m, 1H), 1.18-1.11 (m, 2H), 0.94-0.87 (m, 2H)

### Example 251: N-[5-[4-[[(2R)-1-acetonylazetidin-2-yl]methoxy]-2-methyl-pyrazol-3-yl]pyrazolo[1,5-a]pyridin-2-yl]cyclopropanecarboxamide

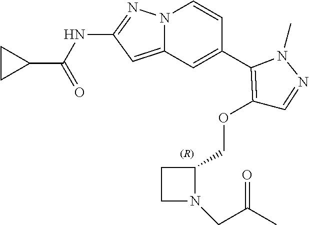

To a mixture of (R)—N-(5-(4-(azetidin-2-ylmethoxy)-1-methyl-1H-pyrazol-5-yl)pyrazolo[1,5-a]pyridin-2-yl)cyclopropanecarboxamide (Example 346, 100 mg, 0.208 mmol), DIEA (80.7 mg, 0.624 mmol) and DMF (3 mL) was added 1-bromopropan-2-one (42.8 mg, 0.312 mmol) at 0° C. The crude material was purified via prep. HPLC using 21-51% CAN-H₂O with 0.04% NH₄OH as modifier (7.9 mg, 8.4%). MS (ESI): mass calcd. for C₂₂H₂₆N₆O₃, 422.2. m/z found, 423.1 [M+H]⁺. ¹H NMR (400 MHz, CDCl₃) δ 8.33 (s, 1H), 8.29 (d, J=7.2 Hz, 1H), 7.47 (s, 1H), 7.33 (s, 1H), 7.00 (s, 1H), 6.82 (dd, J=2.0, 7.2 Hz, 1H), 4.08-3.93 (m, 2H), 3.89 (s, 3H), 3.61-3.45 (m, 3H), 3.25-3.16 (m, 1H), 2.93-2.84 (m, 1H), 2.09-2.00 (m, 2H), 1.98 (s, 3H), 1.63-1.53 (m, 1H), 1.18-1.11 (m, 2H), 0.96-0.87 (m, 2H)

### Example 252: N-[5-[4-[[(2R)-1-[(2R)-2-hydroxypropyl]azetidin-2-yl]methoxy]-2-methyl-pyrazol-3-yl]pyrazolo[1,5-a]pyridin-2-yl]cyclopropanecarboxamide

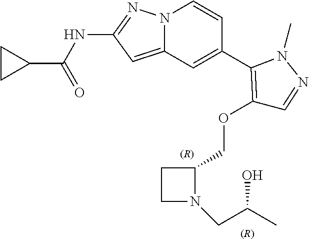

The title compound (27 mg, 37%) was prepared as described in Example 250 where TEA was used instead of DIEA and EtOH was used instead of DMF and (R)-2-methyloxirane was used instead of 1-bromopropan-2-one. MS (ESI): mass calcd. for C₂₂H₂₈N₆O₃, 424.2. m/z found, 425.1 [M+H]⁺. ¹H NMR (400 MHz, CDCl₃) δ 8.48 (s, 1H), 8.31 (d, J=7.2 Hz, 1H), 7.49 (s, 1H), 7.33 (s, 1H), 7.01 (s, 1H), 6.85 (dd, J=1.6, 7.2 Hz, 1H), 4.02-3.90 (m, 2H), 3.89 (s, 3H), 3.69-3.59 (m, 1H), 3.52-3.40 (m, 2H), 3.10-2.95 (m, 2H), 2.56-2.49 (m, 1H), 2.36-2.27 (m, 1H), 2.12-1.94 (m, 2H), 1.63-1.53 (m, 1H), 1.18-1.11 (m, 2H), 0.97 (d, J=6.4 Hz, 3H), 0.94-0.87 (m, 2H)

### Example 253: N-[5-[4-[[(2R)-1-[(2S)-2-hydroxypropyl]azetidin-2-yl]methoxy]-2-methyl-pyrazol-3-yl]pyrazolo[1,5-a]pyridin-2-yl]cyclopropanecarboxamide

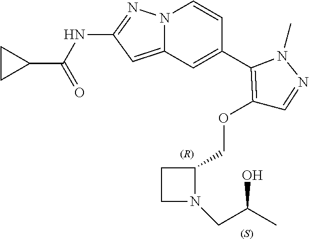

The title compound (12 mg, 12%) was prepared as described in Example 250 where TEA was used instead of DIEA and EtOH was used instead of DMF and (S)-2-methyloxirane was used instead of 1-bromopropan-2-one. MS (ESI): mass calcd. for C₂₂H₂₈N₆O₃, 424.2. m/z found, 425.1 [M+H]⁺. ¹H NMR (400 MHz, CDCl₃) δ 8.40 (s, 1H), 8.31 (d, J=7.2 Hz, 1H), 7.48 (d, J=0.4 Hz, 1H), 7.33 (s, 1H), 7.01 (s, 1H), 6.86 (dd, J=1.6, 7.2 Hz, 1H), 4.00-3.92 (m, 2H), 3.90 (s, 3H), 3.63-3.52 (m, 2H), 3.47-3.40 (m, 1H), 3.28-3.10 (m, 1H), 2.87-2.78 (m, 1H), 2.50-2.42 (m, 1H), 2.39-2.32 (m, 1H), 2.11-1.94 (m, 2H), 1.62-1.53 (m, 1H), 1.18-1.12 (m, 2H), 1.03 (d, J=6.4 Hz, 3H), 0.94-0.88 (m, 2H)

### Example 254: N-[5-[2-methyl-4-[[(2R)-1-(1,1,2,2,2-pentadeuterioethyl)azetidin-2-yl]methoxy]pyrazol-3-yl]pyrazolo[1,5-a]pyridin-2-yl]cyclopropanecarboxamide

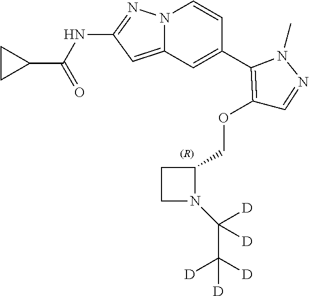

The title compound (25 mg, 20%) was prepared as described in Example 9 where acetaldehyde-d4 was used instead of paraformaldehyde and NaBD₃CN instead of NaBH₃CN. MS (ESI): mass calcd. for C₂₁H₂₁D₅N₆O₂, 399.2. m/z found, 400.1 [M+H]⁺. ¹H NMR (400 MHz, CDCl₃) δ 8.37 (s, 1H), 8.29 (d, J=7.6 Hz, 1H), 7.48 (d, J=0.8 Hz, 1H), 7.33 (s, 1H), 6.99 (s, 1H), 6.84 (dd, J=2.0, 7.2 Hz, 1H), 4.04-3.98 (m, 1H), 3.97-3.92 (m, 1H), 3.90 (s, 3H), 3.44-3.31 (m, 2H), 2.81-2.72 (m, 1H), 2.08-1.91 (m, 2H), 1.62-1.53 (m, 1H), 1.19-1.11 (m, 2H), 0.94-0.87 (m, 2H)

### Example 255: N-[5-[4-[[(2R)-1-(2-cyano-2-methyl-propyl)azetidin-2-yl]methoxy]-2-methyl-pyrazol-3-yl]pyrazolo[1,5-a]pyridin-2-yl]cyclopropanecarboxamide

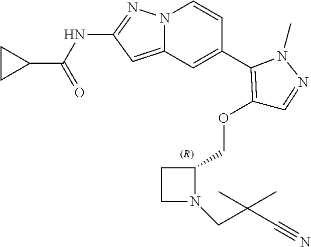

The title compound (63 mg, 34%) was prepared as described in Example 9 where 2,2-dimethyl-3-oxopropanenitrile was used instead of paraformaldehyde. MS (ESI): mass calcd. for C₂₄H₂₉N₇O₂, 447.2. m/z found, 448.2 [M+H]⁺. ¹H NMR (400 MHz, CDCl₃) δ 8.30 (d, J=7.2 Hz, 1H), 8.22 (s, 1H), 7.49-7.42 (m, 1H), 7.33 (s, 1H), 7.00 (s, 1H), 6.85 (dd, J=2.0, 7.2 Hz, 1H), 4.01-3.92 (m, 1H), 3.89 (s, 3H), 3.66-3.52 (m, 2H), 3.06-2.97 (m, 1H), 2.53 (s, 2H), 2.10-1.93 (m, 2H), 1.59-1.54 (m, 1H), 1.17-1.13 (m, 5H), 1.12 (s, 3H), 0.96-0.88 (m, 2H)

### Example 256: N-[5-[4-[[(2R,5R)-1,5-dimethylpyrrolidin-2-yl]methoxy]-2-methyl-pyrazol-3-yl]pyrazolo[1,5-a]pyridin-2-yl]cyclopropanecarboxamide

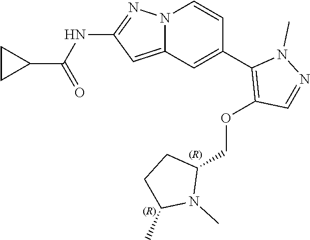

The title compound (50 mg, 3 steps, 13%) was prepared as described in Example 9 where (2R,5R)-tert-butyl 2-methyl-5-((tosyloxy)methyl)pyrrolidine-1-carboxylate was used instead of tert-butyl (S)-2-(hydroxymethyl)azetidine-1-carboxylate in Step A. MS (ESI): mass calcd. for C₂₂H₂₈N₆O₂, 408.2. m/z found, 409.2 [M+H]⁺. ¹H NMR (400 MHz, CD3OD) δ 8.44 (d, J=6.8 Hz, 1H), 7.64 (d, J=1.2 Hz, 1H), 7.44 (s, 1H), 6.96 (dd, J=2.0, 7.2 Hz, 1H), 6.90 (s, 1H), 4.02-3.92 (m, 2H), 3.88 (s, 3H), 2.78-2.65 (m, 1H), 2.39-2.34 (m, 1H), 2.33 (s, 3H), 1.94-1.81 (m, 3H), 1.59-1.48 (m, 1H), 1.43-1.31 (m, 1H), 1.09 (d, J=6.4 Hz, 3H), 1.03-0.97 (m, 2H), 0.93-0.87 (m, 2H)

### Example 257: N-[5-[2-methyl-4-[[(2R,4S)-1-methyl-4-phenyl-pyrrolidin-2-yl]methoxy]pyrazol-3-yl]pyrazolo[1,5-a]pyridin-2-yl]cyclopropanecarboxamide

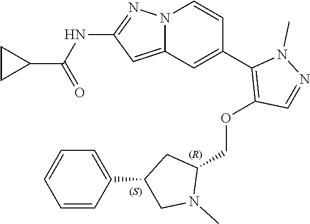

The title compound (28.2 mg, 4 steps, 2.7%) was prepared as described in Example 9, except (2R,4S)-tert-butyl 2-(hydroxymethyl)-4-phenylpyrrolidine-1-carboxylate was used instead of tert-butyl (S)-2-(hydroxymethyl)azetidine-1-carboxylate in Step A. The crude was purified via SFC over AD 250 mm×30 mm, 10 μm (eluent: 35% to 35% (v/v) supercritical 0.1% NH3H2O ETOH). The pure fractions were collected, and the volatiles were removed under reduced pressure. The product was suspended in water (10 mL), the mixture frozen using dry ice/ethyl alcohol and then lyophilized to dryness to afford the titled compound as a white solid. MS (ESI): mass calcd. for C₂₇H₃₀N₆O₂, 470.2. m/z found, 471.1 [M+H]⁺. ¹H NMR (400 MHz, CD3OD) δ 8.40 (d, J=7.2 Hz, 1H), 7.64 (s, 1H), 7.45 (s, 1H), 7.23-7.15 (m, 4H), 7.14-7.08 (m, 1H), 6.94 (dd, J=1.6, 7.2 Hz, 1H), 6.90 (s, 1H), 4.13-4.00 (m, 2H), 3.87 (s, 3H), 3.39-3.32 (m, 1H), 3.10-3.02 (m, 1H), 2.94-2.86 (m, 1H), 2.85-2.77 (m, 1H), 2.43 (s, 3H), 2.42-2.34 (m, 1H), 1.91-1.84 (m, 1H), 1.83-1.74 (m, 1H), 1.03-0.97 (m, 2H), 0.93-0.87 (m, 2H)

### Example 258: N-[5-[2-methyl-4-[[(2R,4R)-1-methyl-4-phenyl-pyrrolidin-2-yl]methoxy]pyrazol-3-yl]pyrazolo[1,5-a]pyridin-2-yl]cyclopropanecarboxamide

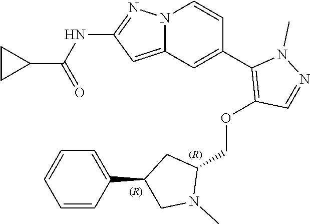

The title compound (110.4 mg, 4 steps, 9.1%) was prepared as described in Example 9, except (2R,4R)-tert-butyl 2-(hydroxymethyl)-4-phenylpyrrolidine-1-carboxylate was used instead of tert-butyl (S)-2-(hydroxymethyl)azetidine-1-carboxylate in Step A. The crude was purified by SFC over AD 250 mm×30 mm, 10 μm (eluent: 35% to 35% (v/v) supercritical 0.1% NH₃H₂O EtOH). The pure fractions were collected, and the volatiles were removed under reduced pressure. The product was suspended in water (10 mL), the mixture frozen using dry ice/ethyl alcohol and then lyophilized to dryness to afford the titled compound as a white solid. MS (ESI): mass calcd. for C₂₇H₃₀N₆O₂, 470.2. m/z found, 471.2 [M+H]⁺. ¹H NMR (400 MHz, DMSO-d6) δ 11.11 (s, 1H), 8.60 (d, J=7.2 Hz, 1H), 7.72 (d, J=0.8 Hz, 1H), 7.49 (s, 1H), 7.32-7.12 (m, 5H), 6.98 (dd, J=2.0, 7.2 Hz, 1H), 6.89 (s, 1H), 4.01-3.93 (m, 1H), 3.92-3.81 (m, 4H), 3.30-3.14 (m, 2H), 2.87-2.78 (m, 1H), 2.39-2.26 (m, 4H), 2.09-1.87 (m, 3H), 0.91-0.74 (m, 4H)

### Example 259: N-(5-(1-methyl-4-(((R)-1-(((R)-tetrahydrofuran-2-yl)methyl)azetidin-2-yl)methoxy)-1H-pyrazol-5-yl)pyrazolo[1,5-a]pyridin-2-yl)cyclopropanecarboxamide

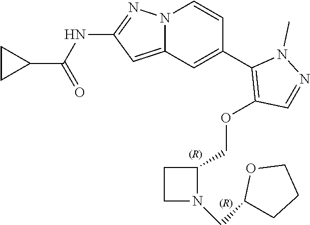

The title compound (57 mg, 4 steps, 28%) was prepared as described in Example 9, except tetrahydrofuran-2-carbaldehyde was used instead of para-formaldehyde in Step D. Racemic material (115 mg, 0.255 mmol) was purified by SFC over DAICEL CHIRALCEL OJ-H 250 mm×30 mm, 5 μm (eluent: 35% to 35% (v/v) supercritical 0.1% NH₃H₂O EtOH). The pure fractions were collected, and the volatiles were removed under reduced pressure. The product was suspended in water (10 mL), the mixture frozen using dry ice/ethanol, and then lyophilized to dryness to afford the title compound as a white solid. MS (ESI): mass calcd. for C₂₄H₃₀N₆O₃, 450.2. m/z found, 451.1 [M+H]⁺. ¹H NMR (400 MHz, DMSO-d6) δ 11.10 (s, 1H), 8.58 (d, J=7.2 Hz, 1H), 7.68 (d, J=0.8 Hz, 1H), 7.44 (s, 1H), 6.95 (dd, J=2.0, 7.2 Hz, 1H), 6.88 (s, 1H), 3.98-3.88 (m, 2H), 3.85 (s, 3H), 3.65-3.54 (m, 2H), 3.49-3.44 (m, 1H), 3.36-3.31 (m, 1H), 3.30-3.24 (m, 1H), 2.85-2.75 (m, 1H), 2.50-2.48 (m, 1H), 2.34-2.24 (m, 1H), 1.98-1.89 (m, 2H), 1.88-1.78 (m, 1H), 1.71-1.62 (m, 1H), 1.62-1.54 (m, 2H), 1.27-1.18 (m, 1H), 0.87-0.78 (m, 4H)

### Example 260: 5-[4-[[(2R)-azetidin-2-yl]methoxy]-2-(difluoromethyl)pyrazol-3-yl]-N-(2,6-dimethylpyrimidin-4-yl)pyrazolo[1,5-a]pyridin-2-amine

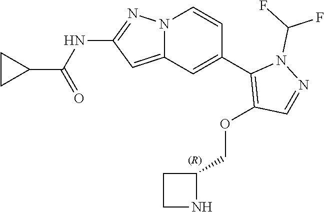

Step A. tert-butyl (R)-2-(((1H-pyrazol-4-yl)oxy)methyl)azetidine-1-carboxylate. A mixture of 1h-pyrazol-4-olms (100 mg, 1.19 mmol), rac-tert-butyl (R)-2-((tosyloxy)methyl)azetidine-1-carboxylate (406 mg, 1.19 mmol), Cs₂CO₃ (852.5 mg, 2.62 mmol), and DMF 1.38 mL was heated to 80° C. for 16 h and 23° C. for 24 h. The reactions mixture was diluted with water (4 mL) and then extracted with 10% MeOH in DCM (3×6 mL) and the organics dried with MgSO4, filtered and concentrated in vacuo. The resulting oil was purified by FCC (12 g GOLD) 0-10% MeOH in DCM to afford the title compound (146 mg, 48%).

Step B. tert-butyl (R)-2-(((1-(difluoromethyl)-1H-pyrazol-4-yl)oxy)methyl)azetidine-1-carboxylate. In a microwave vial a cooled 0° C. solution of tert-butyl (R)-2-(((1H-pyrazol-4-yl)oxy)methyl)azetidine-1-carboxylate (Step A) (290 mg, 1.15) in DMF (5.7 mL) was added NaH (137 mg, 3.44 mmol, 60% in mineral oil). The resulting mixture was stirred for 10 minutes and then was added DIFLUOROIODOMETHANE (6.9 mL, 3.44 mmol) dropwise at 0° C. The mixture was then heated 45° C. for 24 h (with N₂ line removed, since bp of C is 21° C.). The reaction was cooled to 23° C. The mixture was diluted with EtOAc (10 mL) and then was added sat. aq. NaCl (5 mL), slowly. The layers were separated, and the organics were washed with sat. aq. NaCl (5 mL). The organics were dried with MgSO₄, filtered and concentrated in vacuo. The crude yellow oil was purified by flash CC (12 g GOLD) 0-10% MeOH in DCM to afford the titled compound (134 mg, 38.6%). (ESI): mass calcd. for C₁₃H₁₉F₂N₃O₃, 303.3. m/z found, 248 [M+tBu]⁺.

Step C-E. 5-[4-[[(2R)-azetidin-2-yl]methoxy]-2-(difluoromethyl)pyrazol-3-yl]-N-(2,6-dimethylpyrimidin-4-yl)pyrazolo[1,5-a]pyridin-2-amine. Step C-E were described in Example 32 step B-D except tert-butyl (R)-2-(((1-(difluoromethyl)-1H-pyrazol-4-yl)oxy)methyl)azetidine-1-carboxylate from Step B was used instead of tert-butyl (3R,4S)-3-((1-(difluoromethyl)-1H-pyrazol-4-yl)oxy)-4-fluoropyrrolidine-1-carboxylate and Intermediate 3 was used instead of Intermediate 11 to afford the titled compound (80 mg, 3 steps, 41%). (ESI): mass calcd. for C₂₁H₂₂F₂N₈O, 440.2. m/z found, 441.2 [M+H]⁺. ¹H NMR (500 MHz, DMSO-d6) δ 10.23 (s, 1H), 8.64 (dd, J=7.1, 1.0 Hz, 1H), 7.95 (s, 1H), 7.85-7.58 (m, 2H), 6.98 (s, 1H), 6.93 (s, 1H), 6.84 (dd, J=7.2, 2.0 Hz, 1H), 4.04-3.96 (m, 3H), 3.47-3.39 (m, 1H), 3.21-3.14 (m, 1H), 2.46 (s, 3H), 2.30 (s, 3H), 2.22-2.13 (m, 1H), 2.05-1.95 (m, 1H).

### Example 261: (*R)-5-(1-(difluoromethyl)-4-((3-methoxypyrrolidin-3-yl)methoxy)-1H-pyrazol-5-yl)-N-(2,6-dimethylpyrimidin-4-yl)pyrazolo[1,5-a]pyridin-2-amine

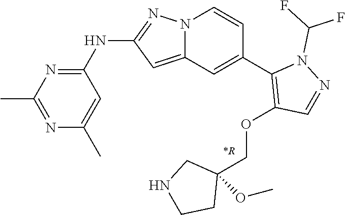 [SMILES: CO[C@]1(COc2cnn(C(F)F)c2-c2ccn3nc(Nc4cc(C)nc(C)n4)cc3c2)CCNC1]

The title compound (67 mg, 4 steps, 10%) was prepared as described in Example 100. The *R enantiomer was obtained by chiral SFC using AD-H (2×25 cm) chiral column, 32% EtOH (0.1% DEA)/CO₂, 100 bar, 65 mL/min. MS (ESI): mass calcd. for C₂₃H₂₆F₂N₈O₂, 484.2. m/z found, 485.2 [M+H]⁺. ¹H NMR (500 MHz, DMSO-d6) δ 10.24 (s, 1H), 8.67 (dd, J=7.2, 1.0 Hz, 1H), 7.99 (s, 1H), 7.90-7.58 (m, 2H), 7.00 (s, 1H), 6.92 (s, 1H), 6.85 (dd, J=7.2, 2.0 Hz, 1H), 4.17 (d, J=10.5 Hz, 1H), 4.09 (d, J=10.5 Hz, 1H), 3.13 (s, 3H), 2.86-2.77 (m, 1H), 2.77-2.70 (m, 3H), 2.46 (s, 3H), 2.30 (s, 3H), 1.80-1.71 (m, 1H), 1.64-1.54 (m, 1H).

### Example 262: (*R)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine

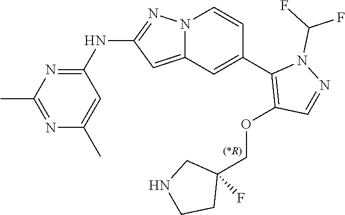 [SMILES: Cc1cc(Nc2cc3cc(-c4c(OC[C@@]5(F)CCNC5)cnn4C(F)F)ccn3n2)nc(C)n1]

The title compound (59 mg, 4 steps, 11%) was prepared as described in Example 32 except tert-butyl 3-fluoro-3-((tosyloxy)methyl)pyrrolidine-1-carboxylate was used instead of tert-butyl (3S,4S)-3-fluoro-4-(tosyloxy)pyrrolidine-1-carboxylate. The *R enantiomer was obtained by chiral SFC using AD-H (2×25 cm) chiral column, 30% MeOH (0.1% DEA)/CO₂, 100 bar, 60 mL/min. MS (ESI): mass calcd. for C₂₂H₂₃F₃N₈O, 472.2. m/z found, 473.2 [M+H]⁺. ¹H NMR (500 MHz, DMSO-d6) δ 10.24 (s, 1H), 8.69-8.64 (m, 1H), 7.98 (s, 1H), 7.87-7.61 (m, 2H), 6.99 (s, 1H), 6.94 (s, 1H), 6.84 (dd, J=7.1, 2.0 Hz, 1H), 4.36-4.22 (m, 2H), 2.94-2.83 (m, 3H), 2.82-2.76 (m, 1H), 2.46 (s, 3H), 2.30 (s, 3H), 1.95-1.76 (m, 2H).

### Example 263: (*S)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine

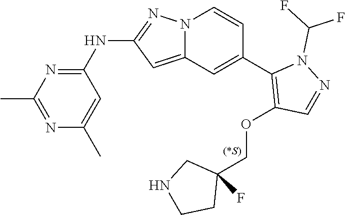 [SMILES: Cc1cc(Nc2cc3cc(-c4c(OC[C@]5(F)CCNC5)cnn4C(F)F)ccn3n2)nc(C)n1]

The title compound (65 mg, 4 steps, 12%) was prepared as described in Example 32 except tert-butyl 3-fluoro-3-((tosyloxy)methyl)pyrrolidine-1-carboxylate was used instead of tert-butyl (3S,4S)-3-fluoro-4-(tosyloxy)pyrrolidine-1-carboxylate. The *S enantiomer was obtained by chiral SFC using AD-H (2×25 cm) chiral column, 30% MeOH (0.1% DEA)/CO₂, 100 bar, 60 mL/min. MS (ESI): mass calcd. for C₂₂H₂₃F₃N₈O, 472.2. m/z found, 473.2 [M+H]⁺. ¹H NMR (500 MHz, DMSO-d6) δ 10.24 (s, 1H), 8.69-8.64 (m, 1H), 7.98 (s, 1H), 7.87-7.61 (m, 2H), 6.99 (s, 1H), 6.94 (s, 1H), 6.84 (dd, J=7.1, 2.0 Hz, 1H), 4.36-4.22 (m, 2H), 2.94-2.83 (m, 3H), 2.82-2.76 (m, 1H), 2.46 (s, 3H), 2.30 (s, 3H), 1.95-1.76 (m, 2H).

### Example 264: 5-[2-(difluoromethyl)-4-[[(2R)-1-ethylazetidin-2-yl]methoxy]pyrazol-3-yl]-N-(5-methylpyrazin-2-yl)pyrazolo[1,5-a]pyridin-2-amine

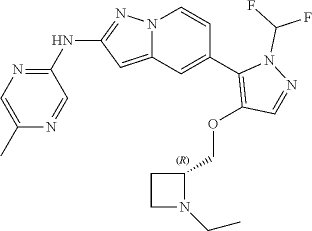

The title compound (36 mg, 3 steps, 13%) was prepared as described in Example 33 except tert-butyl 3-fluoro-3-((tosyloxy)methyl)pyrrolidine-1-carboxylate was used 2-bromo-5-methylpyrazine instead of 6-chloro-2,4-dimethylpyridazin-3(2H)-one in step A and acetaldehyde instead of paraformaldehyde in step B. MS (ESI): mass calcd. for C₂₂H₂₄F₂N₈O, 454.2. m/z found, 455.1 [M+H]⁺. ¹H NMR (400 MHz, DMSO-d6) δ 10.14 (s, 1H), 8.65 (d, J=6.8 Hz, 1H), 8.55 (d, J=1.2 Hz, 1H), 8.11 (s, 1H), 7.96 (s, 1H), 7.92-7.55 (m, 2H), 6.91 (s, 1H), 6.81 (dd, J=2.0, 7.2 Hz, 1H), 4.17-3.93 (m, 2H), 3.33-3.14 (m, 2H), 2.86-2.55 (m, 2H), 2.45-2.17 (m, 4H), 2.09-1.74 (m, 2H), 0.81 (t, J=7.2 Hz, 3H)

### Example 265: 5-[2-methyl-4-[(3S)-1-methylpyrrolidin-3-yl]oxy-pyrazol-3-yl]-N-(6-methylpyrimidin-4-yl)pyrazolo[1,5-a]pyridin-2-amine

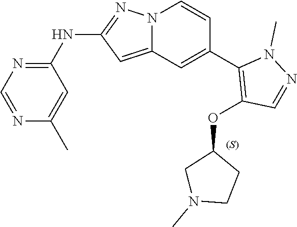

The title compound (73 mg, 36%) was prepared as described in Example 7 except 4-chloro-6-methylpyrimidine was used instead of 2-chloro-6-(trifluoromethyl)pyrazine. MS (ESI): mass calcd. for C₂₁H₂₄N₈O, 404.2. m/z found, 405.3 [M+H]⁺. ¹H NMR (400 MHz, DMSO-d6) δ 10.34 (s, 1H), 8.63 (d, J=7.2 Hz, 1H), 8.59 (s, 1H), 7.69 (s, 1H), 7.40 (s, 1H), 7.14 (br. s., 1H), 6.96-6.89 (m, 2H), 4.73-4.59 (m, 1H), 3.86 (s, 3H), 2.70-2.57 (m, 3H), 2.38-2.25 (m, 4H), 2.24-2.12 (m, 4H), 1.83-1.71 (m, 1H)

### Example 266: 5-[2-methyl-4-[(3S)-1-methylpyrrolidin-3-yl]oxy-pyrazol-3-yl]-N-[6-(trifluoromethyl)-2-pyridyl]pyrazolo[1,5-a]pyridin-2-amine

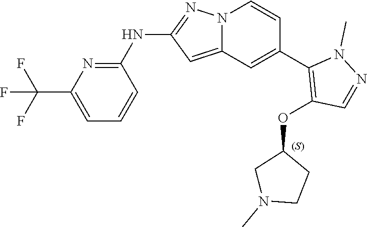

The title compound (90 mg, 40%) was prepared as described in Example 7 except 2-bromo-6-(trifluoromethyl)pyridine was used instead of 2-chloro-6-(trifluoromethyl)pyrazine. MS (ESI): mass calcd. for C₂₂H₂₂F₃N₇O, 457.2. m/z found, 458.2 [M+H]⁺. ¹H NMR (400 MHz, DMSO-d6) δ 10.40 (s, 1H), 8.62 (d, J=7.2 Hz, 1H), 7.92-7.86 (m, 1H), 7.71 (d, J=0.8 Hz, 1H), 7.58 (d, J=8.4 Hz, 1H), 7.40 (s, 1H), 7.27 (d, J=7.2 Hz, 1H), 6.93 (dd, J=2.0, 7.2 Hz, 1H), 6.90 (s, 1H), 4.70-4.64 (m, 1H), 3.87 (s, 3H), 2.68-2.59 (m, 3H), 2.32-2.26 (m, 1H), 2.22 (s, 3H), 2.21-2.13 (m, 1H), 1.82-1.73 (m, 1H) 19F NMR (376 MHz, DMSO-d6) δ −66.98 (s, 3F)

### Example 267: N-(2,6-dimethylpyrimidin-4-yl)-5-[2-methyl-4-[(3S)-1-methylpyrrolidin-3-yl]oxy-pyrazol-3-yl]pyrazolo[1,5-a]pyridin-2-amine

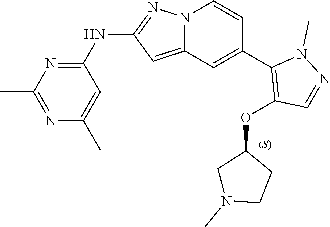

The title compound (52 mg, 38%) was prepared as described in Example 7 except 4-chloro-2,6-dimethylpyrimidine was used instead of 2-chloro-6-(trifluoromethyl)pyrazine. MS (ESI): mass calcd. for C₂₂H₂₆N₈O, 418.2. m/z found, 419.3 [M+H]⁺. ¹H NMR (400 MHz, DMSO-d6) δ 10.22 (s, 1H), 8.62 (d, J=7.2 Hz, 1H), 7.68 (s, 1H), 7.39 (s, 1H), 7.00 (br. s, 1H), 6.95-6.82 (m, 2H), 4.74-4.59 (m, 1H), 3.86 (s, 3H), 2.69-2.64 (m, 1H), 2.63-2.58 (m, 2H), 2.47 (s, 3H), 2.30 (s, 3H), 2.29-2.25 (m, 1H), 2.21 (s, 3H), 2.19-2.12 (m, 1H), 1.84-1.72 (m, 1H)

### Example 268: N-[5-[2-methyl-4-[(3S)-1-methylpyrrolidin-3-yl]oxy-pyrazol-3-yl]pyrazolo[1,5-a]pyridin-2-yl]pyrazolo[1,5-a]pyrimidin-5-amine

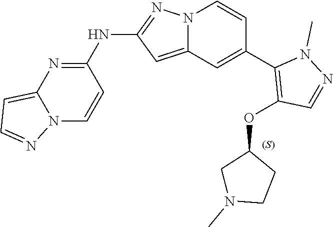

The title compound (9.3 mg, 6.7%) was prepared as described in Example 7 except 5-chloropyrazolo[1,5-a]pyrimidine was used instead of 2-chloro-6-(trifluoromethyl)pyrazine. MS (ESI): mass calcd. for C₂₂H₂₃N₉O, 429.2. m/z found, 430.2 [M+H]⁺. ¹H NMR (400 MHz, CD3OD) δ 8.50 (d, J=7.6 Hz, 1H), 8.44 (d, J=7.2 Hz, 1H), 7.91 (d, J=2.0 Hz, 1H), 7.65 (d, J=0.8 Hz, 1H), 7.36 (s, 1H), 7.24 (s, 1H), 6.93 (dd, J=2.0, 7.2 Hz, 1H), 6.72 (d, J=7.6 Hz, 1H), 6.28-6.24 (m, 1H), 4.75-4.68 (m, 1H), 3.89 (s, 3H), 2.89-2.83 (m, 1H), 2.83-2.77 (m, 1H), 2.76-2.70 (m, 1H), 2.46-2.38 (m, 1H), 2.33 (s, 3H), 2.31-2.21 (m, 1H), 2.02-1.91 (m, 1H)

### Example 269: N-(6,7-dihydro-5H-cyclopenta[c]pyridazin-3-yl)-5-[2-methyl-4-[(3S)-1-methylpyrrolidin-3-yl]oxy-pyrazol-3-yl]pyrazolo[1,5-a]pyridin-2-amine

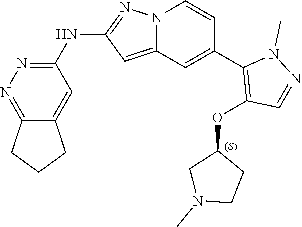

The title compound (14.7 mg, 10.5%) was prepared as described in Example 7 except 3-chloro-6,7-dihydro-5H-cyclopenta[c]pyridazine was used instead of 2-chloro-6-(trifluoromethyl)pyrazine. MS (ESI): mass calcd. for C₂₃H₂₆N₈O, 430.2. m/z found, 431.3 [M+H]⁺. ¹H NMR (400 MHz, DMSO-d6) δ 9.97 (s, 1H), 8.59 (d, J=6.8 Hz, 1H), 7.67 (d, J=0.8 Hz, 1H), 7.47 (s, 1H), 7.39 (s, 1H), 6.91 (s, 1H), 6.88 (dd, J=1.6, 7.2 Hz, 1H), 4.75-4.59 (m, 1H), 3.85 (s, 3H), 2.97 (t, J=7.6 Hz, 2H), 2.91 (t, J=7.6 Hz, 2H), 2.70-2.65 (m, 1H), 2.63-2.58 (m, 2H), 2.36-2.26 (m, 1H), 2.22 (s, 3H), 2.20-2.12 (m, 1H), 2.11-2.00 (m, 2H), 1.83-1.71 (m, 1H)

### Example 270: N-(2-isopropylpyrimidin-4-yl)-5-[2-methyl-4-[(3S)-1-methylpyrrolidin-3-yl]oxy-pyrazol-3-yl]pyrazolo[1,5-a]pyridin-2-amine

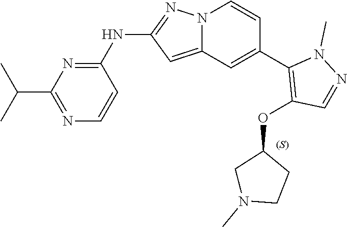

The title compound (21 mg, 12.6%) was prepared as described in Example 7 except 4-chloro-2-isopropylpyrimidine was used instead of 2-chloro-6-(trifluoromethyl)pyrazine. MS (ESI): mass calcd. for C₂₃H₂₈N₈O, 432.2. m/z found, 433.2 [M+H]⁺. ¹H NMR (400 MHz, DMSO-d6) δ 10.33 (s, 1H), 8.62 (d, J=7.2 Hz, 1H), 8.31 (d, J=6.0 Hz, 1H), 7.71 (d, J=1.2 Hz, 1H), 7.40 (s, 1H), 7.11 (br. s., 1H), 6.96 (br. s., 1H), 6.93 (dd, J=2.0, 7.2 Hz, 1H), 4.71-4.64 (m, 1H), 3.86 (s, 3H), 3.06-2.96 (m, 1H), 2.69-2.58 (m, 3H), 2.33-2.26 (m, 1H), 2.22 (s, 3H), 2.19-2.12 (m, 1H), 1.82-1.72 (m, 1H), 1.30 (d, J=6.8 Hz, 6H); t=80° C. ¹H NMR (400 MHz, DMSO-d6, t=80° C.) δ10.06-9.90 (m, 1H), 8.54 (d, J=7.2 Hz, 1H), 8.29 (d, J=6.0 Hz, 1H), 7.65 (s, 1H), 7.34 (s, 1H), 7.12 (d, J=5.6 Hz, 1H), 6.94-6.85 (m, 2H), 4.68-4.59 (m, 1H), 3.83 (s, 3H), 3.02-2.97 (m, 1H), 2.74-2.56 (m, 3H), 2.40-2.30 (m, 1H), 2.22 (s, 3H), 2.19-2.08 (m, 1H), 1.85-1.74 (m, 1H), 1.30 (d, J=6.8 Hz, 6H)

### Example 271: N-(6-isopropylpyrimidin-4-yl)-5-[2-methyl-4-[(3S)-1-methylpyrrolidin-3-yl]oxy-pyrazol-3-yl]pyrazolo[1,5-a]pyridin-2-amine

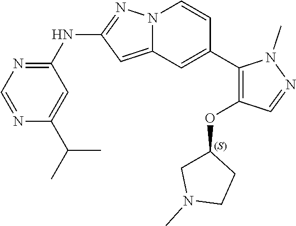

The title compound (45 mg, 32%) was prepared as described in Example 7 except 4-chloro-6-isopropylpyrimidine was used instead of 2-chloro-6-(trifluoromethyl)pyrazine. MS (ESI): mass calcd. for C₂₃H₂₈N₈O, 432.2. m/z found, 433.1 [M+H]⁺. ¹H NMR (400 MHz, DMSO-d6) δ 10.34 (s, 1H), 8.67-8.57 (m, 2H), 7.69 (d, J=0.8 Hz, 1H), 7.40 (s, 1H), 7.13 (br. s., 1H), 6.95 (br. s., 1H), 6.93 (dd, J=2.0, 7.2 Hz, 1H), 4.71-4.63 (m, 1H), 3.85 (s, 3H), 2.90-2.79 (m, 1H), 2.69-2.56 (m, 3H), 2.36-2.24 (m, 1H), 2.21 (s, 3H), 2.20-2.12 (m, 1H), 1.82-1.72 (m, 1H), 1.23 (d, J=6.8 Hz, 6H)

### Example 272: N-(5,6-dimethylpyridazin-3-yl)-5-[2-methyl-4-[(3S)-1-methylpyrrolidin-3-yl]oxy-pyrazol-3-yl]pyrazolo[1,5-a]pyridin-2-amine

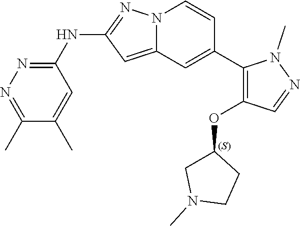

The title compound (36 mg, 26%) was prepared as described in Example 7 except 6-chloro-3,4-dimethylpyridazine was used instead of 2-chloro-6-(trifluoromethyl)pyrazine. MS (ESI): mass calcd. for C₂₂H₂₆N₈O, 418.2. m/z found, 419.2 [M+H]⁺. ¹H NMR (400 MHz, DMSO-d6) δ 9.93 (s, 1H), 8.59 (d, J=7.2 Hz, 1H), 7.66 (d, J=1.2 Hz, 1H), 7.40 (s, 1H), 7.38 (s, 1H), 6.92-6.87 (m, 2H), 4.71-4.63 (m, 1H), 3.86 (s, 3H), 2.69-2.58 (m, 3H), 2.46 (s, 3H), 2.35-2.27 (m, 1H), 2.24 (s, 3H), 2.21 (s, 3H), 2.20-2.13 (m, 1H), 1.82-1.72 (m, 1H)

### Example 273: 5-[4-[[(2R)-azetidin-2-yl]methoxy]-2-methyl-pyrazol-3-yl]-N-[6-methyl-2-(trifluoromethyl)pyrimidin-4-yl]pyrazolo[1,5-a]pyridin-2-amine

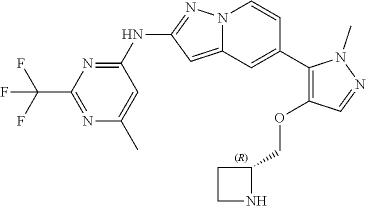

The title compound (241 mg, 2 steps, 82%) was prepared as described in Example 17 Step A and B, except 4-chloro-6-methyl-2-trifluoromethylpyrimidine was used instead of 6-chloro-3,4-dimethylpyridazine. MS (ESI): mass calcd. for C₂₁H₂₁F₃N₈O, 458.2. m/z found, 459.2 [M+H]⁺.

¹H NMR (500 MHz, Chloroform-d) δ 8.41 (d, J=7.1 Hz, 1H), 7.55 (s, 1H), 7.50 (d, J=17.6 Hz, OH), 7.38 (s, 1H), 6.93-6.87 (m, 1H), 6.87 (q, J=6.9, 6.2 Hz, 1H), 4.03 (ddt, J=15.4, 10.9, 6.4 Hz, 2H), 3.91 (d, J=5.2 Hz, 3H), 3.85 (s, 2H), 3.80 (s, 1H), 3.63 (dd, J=15.2, 6.5 Hz, 1H), 3.46 (td, J=8.5, 4.8 Hz, 1H), 3.44-3.32 (m, 1H), 2.53 (d, J=5.1 Hz, 3H), 2.45-2.34 (m, 1H), 2.29-2.17 (m, 1H).

### Example 274: N-(2,6-dimethylpyrimidin-4-yl)-5-[4-[[(2R)-1-(2-fluoroethyl)azetidin-2-yl]methoxy]-2-methyl-pyrazol-3-yl]pyrazolo[1,5-a]pyridin-2-amine

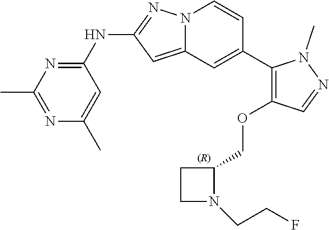

The title compound (230 mg, 3 steps, 52%) was prepared as described in Example 2 Steps A-B then Example 16 Step D, except 1-bromo-2-fluoroethane and potassium carbonate were used instead of iodoethane and cesium carbonate in Example 16 Step D. MS (ESI): mass calcd. for C₂₃H₂₇FN₈O, 450.2. m/z found, 451.2 [M+H]⁺. ¹H NMR (500 MHz, Chloroform-d) δ 8.99 (s, 1H), 8.50-8.45 (m, 1H), 7.39 (dd, J=1.8, 0.9 Hz, 1H), 7.27 (s, 1H), 6.91 (s, 1H), 6.76 (dd, J=7.2, 1.9 Hz, 1H), 6.56 (s, 1H), 4.41-4.19 (m, 2H), 3.94 (dd, J=9.9, 6.7 Hz, 1H), 3.89 (dd, J=9.9, 4.4 Hz, 1H), 3.82 (s, 3H), 3.46-3.34 (m, 2H), 3.00 (s, OH), 2.90-2.82 (m, 1H), 2.85-2.72 (m, 1H), 2.66-2.55 (m, 1H), 2.51 (s, 3H), 2.34 (s, 3H), 2.03-1.89 (m, 2H).

### Example 275: 5-[4-[[(2R)-azetidin-2-yl]methoxy]-2-methyl-pyrazol-3-yl]-N-(5,6-dimethylpyridazin-3-yl)pyrazolo[1,5-a]pyridin-2-amine

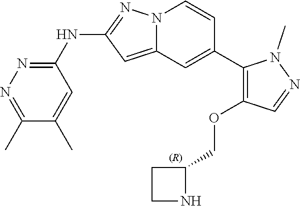

The title compound (420 mg, 2 steps, 60%) was prepared as described in Example 17 Step A and B. MS (ESI): mass calcd. for C₂₁H₂₄N₈O, 404.2. m/z found, 405.2 [M+H]⁺. ¹H NMR (500 MHz, Chloroform-d) δ 8.95 (s, 1H), 8.33-8.27 (m, 1H), 7.67 (s, 1H), 7.31 (dd, J=2.0, 0.9 Hz, 1H), 7.27 (s, 1H), 6.70 (dd, J=7.2, 1.9 Hz, 1H), 6.56 (s, 1H), 5.22 (s, 1H), 4.24-4.17 (m, 1H), 4.00 (dd, J=10.0, 6.6 Hz, 1H), 3.94 (dd, J=10.0, 4.4 Hz, 1H), 3.82 (s, 3H), 3.62 (q, J=8.1 Hz, 1H), 3.46-3.41 (m, 1H), 3.01 (s, 3H), 2.53-2.48 (m, OH), 2.48 (s, 3H), 2.35-2.10 (m, 2H).

### Example 276: N-(5,6-dimethylpyridazin-3-yl)-5-[4-[[(2R)-1-(2-fluoroethyl)azetidin-2-yl]methoxy]-2-methyl-pyrazol-3-yl]pyrazolo[1,5-a]pyridin-2-amine

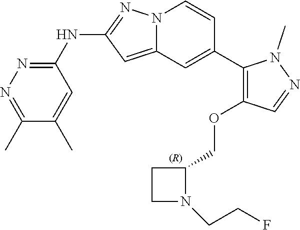

The title compound was prepared by adding 1-bromo-2-fluoroethane (31.4 mg, 0.25 mmol) to a mixture of Example 275 (100 mg, 0.25 mmol), K₂CO₃ (68 mg, 0.49 mmol), and ACN (1 mL). The mixture was stirred at 90° C. for 1 h. The crude was filtered and purified via prep HPLC to afford a white solid (104 mg, 93.4%). MS (ESI): mass calcd. for C₂₃H₂₇FN₈O, 450.2. m/z found, 451.3 [M+H]⁺. ¹H NMR (500 MHz, Chloroform-d) δ 10.04 (s, 1H), 8.45 (d, J=7.1 Hz, 1H), 7.86 (s, 1H), 7.43 (dd, J=1.9, 0.9 Hz, 1H), 7.33 (s, 1H), 6.80-6.73 (m, 2H), 4.49-4.27 (m, 1H), 4.01 (dd, J=9.9, 6.7 Hz, 1H), 3.96 (dd, J=9.8, 4.4 Hz, 1H), 3.89 (s, 3H), 3.53-3.44 (m, 3H), 2.93 (td, J=7.9, 6.8, 5.6 Hz, 1H), 2.93-2.79 (m, 1H), 2.74-2.62 (m, 1H), 2.58 (s, 3H), 2.33-2.29 (m, 3H), 2.11-1.96 (m, 2H).

### Example 277: 5-[4-[[(2R)-1-(2-fluoroethyl)azetidin-2-yl]methoxy]-2-methyl-pyrazol-3-yl]-N-(6-methylpyrimidin-4-yl)pyrazolo[1,5-a]pyridin-2-amine

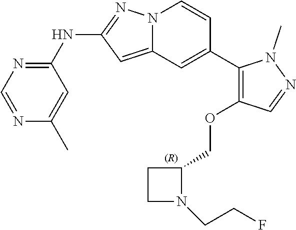

The title compound was prepared by adding 1-bromo-2-fluoroethane (32 mg, 0.26 mmol) to a mixture of (R)-5-(4-(azetidin-2-ylmethoxy)-1-methyl-1H-pyrazol-5-yl)-N-(6-methylpyrimidin-4-yl)pyrazolo[1,5-a]pyridin-2-amine (100 mg, 0.25 mmol), prepared similarly as Example 17 step A and B where 4-BROMO-6-METHYLPYRIMIDINE was used instead of 6-chloro-3,4-dimethylpyridazine, K₂CO₃ (70 mg, 0.51 mmol), and ACN (1 mL). The mixture was stirred at 90° C. for 1 h. The crude was filtered and purified via prep HPLC to afford a white solid (107 mg, 96%). MS (ESI): mass calcd. for C₂₂H₂₅FN₈O, 436.2. m/z found, 437.2 [M+H]⁺. ¹H NMR (500 MHz, Chloroform-d) δ 9.10 (s, 1H), 8.68 (d, J=1.1 Hz, 1H), 8.38 (dt, J=7.1, 0.9 Hz, 1H), 7.40 (dd, J=1.9, 0.9 Hz, 1H), 7.28 (s, 1H), 7.20 (s, 1H), 6.78 (dd, J=7.2, 1.9 Hz, 1H), 6.52 (s, 1H), 4.43-4.21 (m, 2H), 3.95 (dd, J=9.9, 6.7 Hz, 1H), 3.90 (dd, J=9.8, 4.4 Hz, 1H), 3.83 (s, 3H), 3.48-3.38 (m, 2H), 2.92-2.83 (m, 1H), 2.87-2.73 (m, 1H), 2.68-2.57 (m, 1H), 2.41 (d, J=0.6 Hz, 3H), 2.04-1.96 (m, 1H), 2.00-1.90 (m, 1H).

### Example 278: 5-[4-[[(2R)-1-(2-fluoroethyl)azetidin-2-yl]methoxy]-2-methyl-pyrazol-3-yl]-N-(6-methylpyrazin-2-yl)pyrazolo[1,5-a]pyridin-2-amine

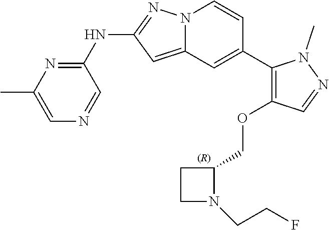

The title compound was prepared by adding 1-bromo-2-fluoroethane (48.7 mg, 0.38 mmol) to a mixture of (R)-5-(4-(azetidin-2-ylmethoxy)-1-methyl-1H-pyrazol-5-yl)-N-(6-methylpyrimidin-4-yl)pyrazolo[1,5-a]pyridin-2-amine (150 mg, 0.38 mmol), prepared similarly as Example 17 step A and B where 2-BROMO-6-METHYLPYRAZINE was used instead of 6-chloro-3,4-dimethylpyridazine, K₂CO₃ (106 mg, 0.77 mmol), and ACN (10 mL). The mixture was stirred at 90° C. for 1 h. The crude was filtered and purified via prep HPLC to afford a white solid (136 mg, 81%). MS (ESI): mass calcd. for C₂₂H₂₅FN₈O, 436.2. m/z found, 437.2 [M+H]⁺. ¹H NMR (400 MHz, Chloroform-d) δ 8.51 (s, 1H), 8.41 (dt, J=7.1, 0.9 Hz, 1H), 7.98 (s, 1H), 7.92 (s, 1H), 7.48 (dd, J=1.9, 0.9 Hz, 1H), 7.37 (s, 1H), 6.86-6.74 (m, 2H), 4.55-4.40 (m, 1H), 4.43-4.28 (m, 1H), 4.08-3.94 (m, 2H), 3.92 (s, 3H), 3.50 (td, J=7.4, 3.7 Hz, 2H), 3.02-2.80 (m, 2H), 2.79-2.64 (m, 1H), 2.51 (s, 3H), 2.15-1.97 (m, 2H).

### Example 279: 5-[4-[[(2R)-1-(2-fluoroethyl)azetidin-2-yl]methoxy]-2-methyl-pyrazol-3-yl]-N-(5-methylpyrazin-2-yl)pyrazolo[1,5-a]pyridin-2-amine

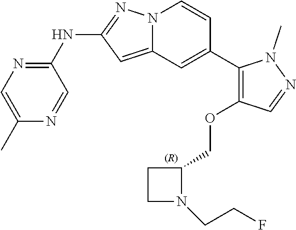

The title compound was prepared by adding 1-bromo-2-fluoroethane (32 mg, 0.26 mmol) to a mixture of (R)-5-(4-(azetidin-2-ylmethoxy)-1-methyl-1H-pyrazol-5-yl)-N-(6-methylpyrimidin-4-yl)pyrazolo[1,5-a]pyridin-2-amine (100 mg, 0.25 mmol), prepared similarly as Example 17 step A and B where 2-chloro-5-methylpyrazine was used instead of 6-chloro-3,4-dimethylpyridazine, K₂CO₃ (70 mg, 0.51 mmol), and ACN (1 mL). The mixture was stirred at 90° C. for 1 h. The crude was filtered and purified via prep HPLC to afford a white solid (81 mg, 72%). MS (ESI): mass calcd. for C₂₂H₂₅FN₈O, 436.2. m/z found, 437.2 [M+H]⁺. ¹H NMR (400 MHz, Chloroform-d) δ 8.63 (d, J=1.5 Hz, 1H), 8.40 (dt, J=7.1, 0.9 Hz, 1H), 8.27 (t, J=2.5 Hz, 1H), 8.10 (dd, J=1.5, 0.7 Hz, 1H), 7.45 (dd, J=1.9, 0.9 Hz, 1H), 7.36 (s, 1H), 6.81 (dd, J=7.2, 1.9 Hz, 1H), 6.70 (d, J=0.8 Hz, 1H), 4.53-4.39 (m, 1H), 4.42-4.27 (m, 1H), 4.07-3.93 (m, 2H), 3.90 (s, 3H), 3.56-3.45 (m, 2H), 3.01-2.91 (m, 1H), 2.96-2.79 (m, 1H), 2.77-2.63 (m, 1H), 2.49 (s, 3H), 2.14-1.97 (m, 2H).

### Example 280: N-[5-[2-methyl-4-[(3S)-1-methylpyrrolidin-3-yl]oxy-pyrazol-3-yl]pyrazolo[1,5-a]pyridin-2-yl]-7,8-dihydro-5H-pyrano[4,3-b]pyridin-2-amine

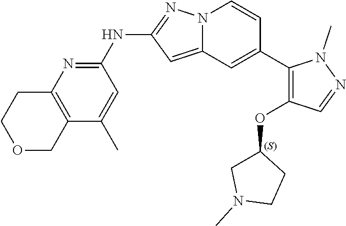

The title compound (63 mg, 29%) was prepared as described in Example 7 except 2-chloro-7,8-dihydro-5H-pyrano[4,3-b]pyridine was used instead of 2-chloro-6-(trifluoromethyl)pyrazine. MS (ESI): mass calcd. for C₂₄H₂₇N₇O₂, 445.2. m/z found, 446.1 [M+H]⁺. ¹H NMR (400 MHz, DMSO-d6) δ 9.73 (s, 1H) 8.55 (d, J=7.2 Hz, 1H) 7.61 (d, J=0.4 Hz, 1H) 7.38 (s, 1H) 7.32 (d, J=8.8 Hz, 1H) 7.19 (d, J=8.4 Hz, 1H) 6.82-6.89 (m, 2H) 4.63-4.69 (m, 1H) 4.61 (s, 2H) 3.96 (t, J=5.6 Hz, 2H) 3.85 (s, 3H) 2.82 (t, J=5.6 Hz, 2H) 2.63-2.70 (m, 1H) 2.57-2.63 (m, 2H) 2.25-2.34 (m, 1H) 2.21 (s, 3H) 2.12-2.20 (m, 1H) 1.72-1.82 (m, 1H)

### Example 281: N-(2-cyclopropylpyrimidin-4-yl)-5-[2-methyl-4-[(3S)-1-methylpyrrolidin-3-yl]oxy-pyrazol-3-yl]pyrazolo[1,5-a]pyridin-2-amine

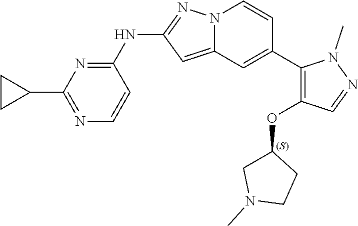

The title compound (81 mg, 59%) was prepared as described in Example 7 except 4-chloro-2-cyclopropylpyrimidine was used instead of 2-chloro-6-(trifluoromethyl)pyrazine. MS (ESI): mass calcd. for C₂₃H₂₆N₈O, 430.2. m/z found, 431.1 [M+H]⁺. ¹H NMR (400 MHz, DMSO-d6) δ 10.29 (s, 1H), 8.62 (d, J=6.8 Hz, 1H), 8.21 (d, J=5.6 Hz, 1H), 7.70 (d, J=0.8 Hz, 1H), 7.40 (s, 1H), 7.04 (s, 1H), 6.93 (dd, J=2.0, 7.2 Hz, 1H), 6.89 (s, 1H), 4.70-4.64 (m, 1H), 3.86 (s, 3H), 2.68-2.59 (m, 3H), 2.34-2.26 (m, 1H), 2.22 (s, 3H), 2.21-2.13 (m, 1H), 2.12-2.04 (m, 1H), 1.82-1.73 (m, 1H), 1.06-0.96 (m, 4H)

### Example 282: N-(2,6-dimethylpyrimidin-4-yl)-5-[2-methyl-4-[[(2R)-1-methylpyrrolidin-2-yl]methoxy]pyrazol-3-yl]pyrazolo[1,5-a]pyridin-2-amine

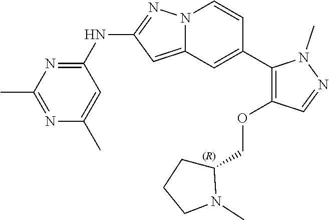

The title compound (84 mg, 31%) was prepared as described in Example 7 except 4-chloro-2,6-dimethylpyrimidine was used instead of 2-chloro-6-(trifluoromethyl)pyrazine. MS (ESI): mass calcd. for C₂₃H₂₈N₈O, 432.2. m/z found, 433.3 [M+H]⁺. ¹H NMR (400 MHz, DMSO-d6) δ 10.23 (s, 1H), 8.62 (d, J=7.2 Hz, 1H), 7.69 (s, 1H), 7.47 (s, 1H), 7.01 (br s, 1H), 6.93 (dd, J=2.0, 7.2 Hz, 1H), 6.88 (s, 1H), 3.97-3.91 (m, 1H), 3.87 (s, 3H), 3.84-3.77 (m, 1H), 2.96-2.86 (m, 1H), 2.49-2.42 (m, 4H), 2.30 (s, 3H), 2.29 (s, 3H), 2.19-2.10 (m, 1H), 1.92-1.82 (m, 1H), 1.68-1.59 (m, 2H), 1.58-1.48 (m, 1H)

### Example 283: 5-[2-methyl-4-[[(2R)-1-methylpyrrolidin-2-yl]methoxy]pyrazol-3-yl]-N-[6-methyl-2-(trifluoromethyl)pyrimidin-4-yl]pyrazolo[1,5-a]pyridin-2-amine

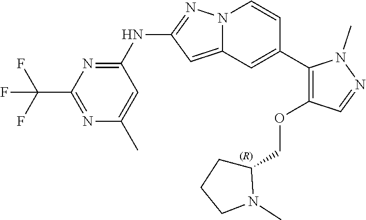

Step A. (R)—N-(6-methyl-2-(trifluoromethyl)pyrimidin-4-yl)-5-(1-methyl-4-(pyrrolidin-2-ylmethoxy)-1H-pyrazol-5-yl)pyrazolo[1,5-a]pyridin-2-amine. The title compound (480 mg, 100%) was prepared as described in Example 7 except 4-chloro-6-methyl-2-(trifluoromethyl)pyrimidine was used instead of 2-chloro-6-(trifluoromethyl)pyrazine and (R)-tert-butyl 2-(((5-(2-aminopyrazolo[1,5-a]pyridin-5-yl)-1-methyl-1H-pyrazol-4-yl)oxy)methyl)pyrrolidine-1-carboxylate was used instead of Intermediate 7.

Step B. 5-[2-methyl-4-[[(2R)-1-methylpyrrolidin-2-yl]methoxy]pyrazol-3-yl]-N-[6-methyl-2-(trifluoromethyl)pyrimidin-4-yl]pyrazolo[1,5-a]pyridin-2-amine. The title compound (118 mg, 28%) was prepared similarly as describe Example 23. MS (ESI): mass calcd. for C₂₃H₂₅F₃N₈O, 486.2. m/z found, 487.1 [M+H]⁺. ¹H NMR (400 MHz, DMSO-d6) δ 10.94 (s, 1H), 8.67 (d, J=7.2 Hz, 1H), 7.77 (s, 1H), 7.47 (s, 1H), 7.37 (br.s., 1H), 7.00 (dd, J=2.0, 7.2 Hz, 1H), 6.86 (br.s., 1H), 3.97-3.92 (m, 1H), 3.88 (s, 3H), 3.85-3.79 (m, 1H), 2.98-2.82 (m, 1H), 2.49-2.47 (m, 1H), 2.46 (s, 3H), 2.29 (s, 3H), 2.21-2.08 (m, 1H), 1.94-1.80 (m, 1H), 1.68-1.59 (m, 2H), 1.59-1.50 (m, 1H)

### Example 284: N-(6-cyclopropylpyridazin-3-yl)-5-[2-methyl-4-[[(2R)-1-methylpyrrolidin-2-yl]methoxy]pyrazol-3-yl]pyrazolo[1,5-a]pyridin-2-amine

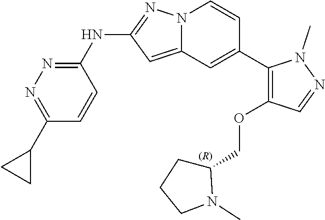

The title compound (60 mg, 3 steps, 14%) was prepared as described in Example 283 except 3-chloro-6-cyclopropylpyridazine was used instead of 4-chloro-6-methyl-2-(trifluoromethyl)pyrimidine. MS (ESI): mass calcd. for C₂₄H₂₈N₈O, 444.2. m/z found, 445.2 [M+H]⁺. ¹H NMR (400 MHz, DMSO-d6) δ 10.02 (s, 1H), 8.59 (d, J=7.2 Hz, 1H), 7.67 (d, J=1.2 Hz, 1H), 7.52 (d, J=9.2 Hz, 1H), 7.47 (s, 1H), 7.34 (d, J=9.2 Hz, 1H), 6.93-6.88 (m, 2H), 3.98-3.90 (m, 1H), 3.86 (s, 3H), 3.84-3.78 (m, 1H), 2.97-2.86 (m, 1H), 2.56-2.53 (m, 1H), 2.30 (s, 3H), 2.21-2.10 (m, 2H), 1.95-1.83 (m, 1H), 1.69-1.59 (m, 2H), 1.58-1.47 (m, 1H), 1.04-0.97 (m, 2H), 0.97-0.91 (m, 2H)

### Example 285: 5-[2-methyl-4-[[(2R)-1-methylpyrrolidin-2-yl]methoxy]pyrazol-3-yl]-N-(6-methylpyrimidin-4-yl)pyrazolo[1,5-a]pyridin-2-amine

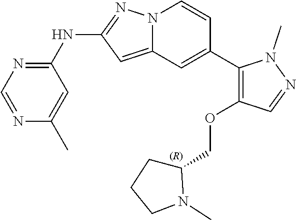

The title compound (95 mg, 3 steps, 9.7%) was prepared as described in Example 283 except 4-chloro-6-methylpyrimidine was used instead of 4-chloro-6-methyl-2-(trifluoromethyl)pyrimidine. MS (ESI): mass calcd. for C₂₂H₂₆N₈O, 418.2. m/z found, 419.1 [M+H]⁺. ¹H NMR (400 MHz, DMSO-d6) δ 10.34 (s, 1H), 8.63 (d, J=7.2 Hz, 1H), 8.59 (s, 1H), 7.70 (d, J=1.2 Hz, 1H), 7.47 (s, 1H), 7.16 (br s, 1H), 6.95 (dd, J=2.0, 7.2 Hz, 1H), 6.91 (s, 1H), 3.98-3.91 (m, 1H), 3.87 (s, 3H), 3.85-3.74 (m, 1H), 2.96-2.88 (m, 1H), 2.50-2.45 (m, 1H), 2.35 (s, 3H), 2.29 (s, 3H), 2.18-2.09 (m, 1H), 1.93-1.82 (m, 1H), 1.68-1.59 (m, 2H), 1.59-1.47 (m, 1H)

### Example 286: 5-[2-methyl-4-[[(2R)-1-methylpyrrolidin-2-yl]methoxy]pyrazol-3-yl]-N-(2-methylpyrimidin-4-yl)pyrazolo[1,5-a]pyridin-2-amine

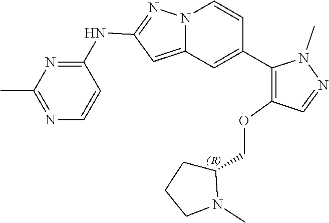

The title compound (77 mg, 3 steps, 21%) was prepared as described in Example 283 except 4-chloro-2-methylpyrimidine was used instead of 4-chloro-6-methyl-2-(trifluoromethyl)pyrimidine. MS (ESI): mass calcd. for C₂₂H₂₆N₈O, 418.2. m/z found, 419.1 [M+H]⁺. ¹H NMR (400 MHz, DMSO-d6) δ 10.35 (s, 1H), 8.62 (d, J=7.2 Hz, 1H), 8.28 (d, J=6.0 Hz, 1H), 7.71 (d, J=0.8 Hz, 1H), 7.47 (s, 1H), 7.17 (br.s., 1H), 6.95 (dd, J=2.0, 7.2 Hz, 1H), 6.89 (br.s., 1H), 3.97-3.91 (m, 1H), 3.87 (s, 3H), 3.85-3.79 (m, 1H), 2.97-2.85 (m, 1H), 2.50-2.46 (m, 4H), 2.29 (s, 3H), 2.20-2.08 (m, 1H), 1.96-1.81 (m, 1H), 1.70-1.59 (m, 2H), 1.58-1.48 (m, 1H)

### Example 287: 5-[2-methyl-4-[[(2R)-1-methylpyrrolidin-2-yl]methoxy]pyrazol-3-yl]-N-(6-methylpyridazin-3-yl)pyrazolo[1,5-a]pyridin-2-amine

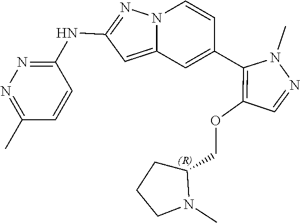

The title compound (55 mg, 3 steps, 4.5%) was prepared as described in Example 283 except 3-chloro-6-methylpyridazine was used instead of 4-chloro-6-methyl-2-(trifluoromethyl)pyrimidine. MS (ESI): mass calcd. for C₂₂H₂₆N₈O, 418.2. m/z found, 419.1 [M+H]⁺. ¹H NMR (400 MHz, DMSO-d6) δ 10.05 (s, 1H), 8.59 (d, J=7.2 Hz, 1H), 7.68 (s, 1H), 7.58 (d, J=9.2 Hz, 1H), 7.47 (s, 1H), 7.42 (d, J=9.2 Hz, 1H), 6.91 (dd, J=1.6, 7.2 Hz, 1H), 6.88 (s, 1H), 3.96-3.92 (m, 1H), 3.87 (s, 3H), 3.84-3.80 (m, 1H), 3.04-3.00 (m, 1H), 2.94-2.88 (m, 1H), 2.53-2.52 (m, 3H), 2.29 (s, 3H), 2.19-2.10 (m, 1H), 1.95-1.83 (m, 1H), 1.69-1.60 (m, 2H), 1.59-1.49 (m, 1H)

### Example 288: 5-[4-(3-azabicyclo[2.1.1]hexan-4-ylmethoxy)-2-methyl-pyrazol-3-yl]-N-(2,6-dimethylpyrimidin-4-yl)pyrazolo[1,5-a]pyridin-2-amine

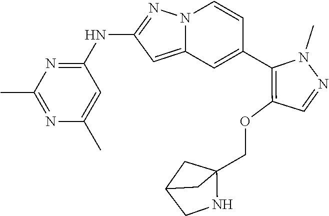

The title compound (47 mg, 3 steps, 42%) was prepared as described in Example 386 Steps A-B then Example 8 Step C, except (2-benzyl-2-azabicyclo[2.1.1]hexan-1-yl)methanol was used instead of tert-butyl-3-hydroxycyclobutylcarbamate in Step A and Intermediate 4 was used instead of 3-hydroxy-5-methylisoxazole in Step B. MS (ESI): mass calcd. for C₂₃H₂₆N₈O, 430.2. m/z found, 431.1 [M+H]⁺. ¹H NMR (400 MHz, DMSO-d6) δ 15.07 (br. s., 1H), 12.15-11.66 (m, 1H), 9.99-9.64 (m, 2H), 8.89-8.58 (m, 1H), 8.03-7.88 (m, 1.5H), 7.58 (s, 1H), 7.37-7.28 (m, 0.5H), 7.19 (d, J=6.8 Hz, 1H), 7.03-6.84 (m, 0.7H), 6.67-6.47 (m, 0.3H), 4.39 (s, 2H), 3.90 (s, 3H), 3.34-3.26 (m, 2H), 2.87-2.82 (m, 1H), 2.80-2.55 (m, 6H), 2.07-1.98 (m, 2H), 1.67-1.57 (m, 2H)

### Example 289: N-(2,6-dimethylpyrimidin-4-yl)-5-[4-[[(2R)-1-ethylpyrrolidin-2-yl]methoxy]-2-methyl-pyrazol-3-yl]pyrazolo[1,5-a]pyridin-2-amine

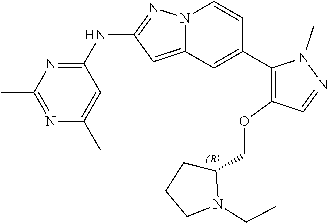

The title compound (101.2 mg, 45%) was prepared as described in Example 1 Step F, except (R)—N-(2,6-dimethylpyrimidin-4-yl)-5-(1-methyl-4-(pyrrolidin-2-ylmethoxy)-1H-pyrazol-5-yl)pyrazolo[1,5-a]pyridin-2-amine was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₄H₃₀N₈O, 446.3. m/z found, 447.2 [M+H]⁺. ¹H NMR (400 MHz, CDCl₃) δ 8.40 (d, J=7.2 Hz, 1H), 7.51 (s, 1H), 7.47 (d, J=0.8 Hz, 1H), 7.35 (s, 1H), 7.06 (s, 1H), 6.86 (dd, J=1.6, 7.2 Hz, 1H), 6.60 (s, 1H), 4.07-3.98 (m, 1H), 3.92 (s, 3H), 3.88-3.80 (m, 1H), 3.24-3.15 (m, 1H), 3.00-2.90 (m, 1H), 2.87-2.79 (m, 1H), 2.61 (s, 3H), 2.45 (s, 3H), 2.43-2.35 (m, 1H), 2.32-2.21 (m, 1H), 2.00-1.91 (m, 1H), 1.77-1.63 (m, 3H), 1.10 (t, J=7.6 Hz, 3H)

### Example 290: N-(2,6-dimethylpyrimidin-4-yl)-5-[2-methyl-4-[[(2R)-1-(trideuteriomethyl)azetidin-2-yl]methoxy]pyrazol-3-yl]pyrazolo[1,5-a]pyridin-2-amine

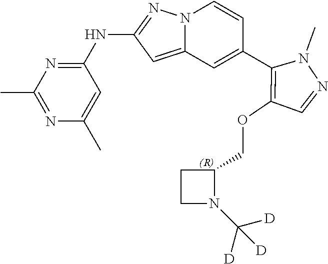

The title compound (72.6 mg, 46%) was prepared as described in Example 1 Step F, except 5-[4-[[(2R)-azetidin-2-yl]methoxy]-2-methyl-pyrazol-3-yl]-N-(2,6-dimethylpyrimidin-4-yl)pyrazolo[1,5-a]pyridin-2-amine (Example 43) was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide, PARAFORMALDEHYDE-D2 was used instead of paraformaldehyde, and sodium cyanoborodeutride was used instead of sodium cyanoborohydride. MS (ESI): mass calcd. for C₂₂H₂₆N₈O, 421.2. m/z found, 422.3 [M+H]⁺. ¹H NMR (500 MHz, DMSO-d6) δ 10.19 (s, 1H), 8.61 (dt, J=7.1, 0.9 Hz, 1H), 7.68 (dd, J=2.0, 0.9 Hz, 1H), 7.44 (s, 1H), 7.00 (s, 1H), 6.93 (dd, J=7.2, 2.0 Hz, 1H), 6.86 (s, 1H), 3.97-3.89 (m, 2H), 3.86 (s, 3H), 3.27-3.20 (m, 2H), 2.71 (q, J=7.8 Hz, 1H), 2.46 (s, 3H), 2.30 (s, 3H), 1.98-1.89 (m, 1H), 1.89-1.79 (m, 1H).

### Example 291: 5-[4-[[(2R)-azetidin-2-yl]methoxy]-2-methyl-pyrazol-3-yl]-N-(6-pyrazol-1-ylpyrazin-2-yl)pyrazolo[1,5-a]pyridin-2-amine

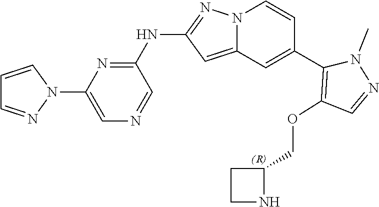

The title compound (380 mg, 2 steps, 68%) was prepared as described in Example 2 Step A-B, except 2-chloro-6-(1h-pyrazol-1-yl)pyrazine was used instead of 2,6-dimethylpyrimidine in Step A. MS (ESI): mass calcd. for C₂₂H₂₂N₁₀O, 442.2. m/z found, 443.2 [M+H]⁺. ¹H NMR (500 MHz, Chloroform-d) δ 8.73 (s, 1H), 8.51 (s, 1H), 8.42 (dd, J=2.6, 0.7 Hz, 1H), 8.29 (dt, J=7.2, 0.9 Hz, 1H), 7.75-7.69 (m, 1H), 7.49-7.42 (m, 2H), 7.30 (s, 1H), 6.80 (dd, J=7.2, 1.9 Hz, 1H), 6.64 (d, J=0.8 Hz, 1H), 6.46 (dd, J=2.6, 1.7 Hz, 1H), 4.15-4.10 (m, 1H), 4.01-3.88 (m, 2H), 3.86 (s, 3H), 3.57 (q, J=7.9 Hz, 1H), 3.42 (s, 1H), 3.39-3.33 (m, 1H), 2.32-2.21 (m, 1H), 2.16-2.07 (m, 1H).

### Example 292: (2S)-1-[(2R)-2-[[5-[2-[(2,6-dimethylpyrimidin-4-yl)amino]pyrazolo[1,5-a]pyridin-5-yl]-1-methyl-pyrazol-4-yl]oxymethyl]azetidin-1-yl]propan-2-ol

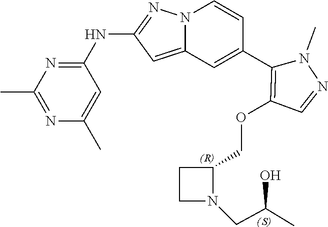

The title compound (41.3 mg, 18%) was prepared as described in Example 56 Step C, except (S)-2-methyloxirane was used instead of acetyl chloride and (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine was used instead of (R)—N-(5-(4-(azetidin-2-ylmethoxy)-1-methyl-1H-pyrazol-5-yl)pyrazolo[1,5-a]pyridin-2-yl)cyclopropanecarboxamide. MS (ESI): mass calcd. for C₂₄H₃₀N₈O₂, 462.2. m/z found, 463.2 [M+H]⁺. ¹H NMR (400 MHz, CDCl₃) δ 8.39 (d, J=6.8 Hz, 1H), 7.47 (d, J=0.8 Hz, 1H), 7.40 (s, 1H), 7.34 (s, 1H), 7.06 (s, 1H), 6.86 (dd, J=1.6, 7.2 Hz, 1H), 6.61 (s, 1H), 4.02-3.93 (m, 2H), 3.91 (s, 3H), 3.64-3.54 (m, 2H), 3.49-3.42 (m, 1H), 3.24 (br. s., 1H), 2.88-2.79 (m, 1H), 2.60 (s, 3H), 2.52-2.43 (m, 4H), 2.40-2.34 (m, 1H), 2.12-1.95 (m, 2H), 1.05 (d, J=6.4 Hz, 3H)

### Example 293: N-(2,6-dimethylpyrimidin-4-yl)-5-[2-methyl-4-[[(2R)-1-(1,1,2,2,2-pentadeuterioethyl)pyrrolidin-2-yl]methoxy]pyrazol-3-yl]pyrazolo[1,5-a]pyridin-2-amine

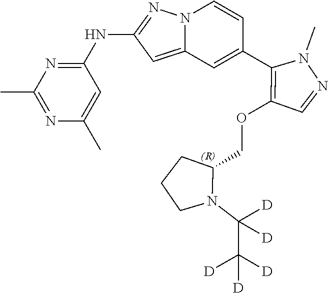

The title compound (75.1 mg, 34%) was prepared as described in Example 1 Step F, except (R)—N-(2,6-dimethylpyrimidin-4-yl)-5-(1-methyl-4-(pyrrolidin-2-ylmethoxy)-1H-pyrazol-5-yl)pyrazolo[1,5-a]pyridin-2-amine was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide, CD₃CDO was used instead of paraformaldehyde, sodium cyanoborodeutride was used instead of sodium cyanoborohydride, and deuterated methanol was used instead of methanol. MS (ESI): mass calcd. for C₂₄H₂₅D₅N₈O, 451.3. m/z found, 452.2 [M+H]⁺. ¹H NMR (400 MHz, CDCl₃) δ 8.43 (d, J=7.2 Hz, 1H), 8.22 (br. s 0.1H), 7.47 (d, J=0.8 Hz, 1H), 7.35 (s, 1H), 7.05 (s, 1H), 6.85 (dd, J=2.0, 7.2 Hz, 1H), 6.65 (s, 1H), 4.02-3.96 (m, 1H), 3.92 (s, 3H), 3.87-3.81 (m, 1H), 3.21-3.14 (m, 1H), 2.88-2.79 (m, 1H), 2.61 (s, 3H), 2.44 (s, 3H), 2.31-2.21 (m, 1H), 2.01-1.89 (m, 1H), 1.86-1.73 (m, 2H), 1.72-1.62 (m, 1H)

### Example 294: N-(2,6-dimethylpyrimidin-4-yl)-5-[2-methyl-4-[[(2R)-1-(1,1,2,2,2-pentadeuterioethyl)azetidin-2-yl]methoxy]pyrazol-3-yl]pyrazolo[1,5-a]pyridin-2-amine

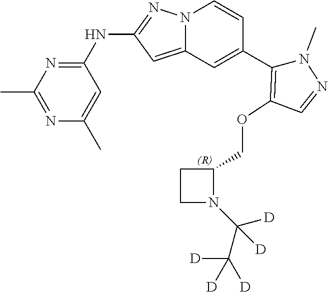

The title compound (100.8 mg, 57%) was prepared as described in Example 1 Step F, except (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide, CD₃CDO was used instead of paraformaldehyde, sodium cyanoborodeutride was used instead of sodium cyanoborohydride, and deuterated methanol was used instead of methanol. MS (ESI): mass calcd. for C₂₃H₂₃D₅N₈O, 437.3. m/z found, 438.2 [M+H]⁺. ¹H NMR (400 MHz, CDCl₃) δ 8.38 (d, J=7.2 Hz, 1H), 7.52-7.43 (m, 2H), 7.35 (s, 1H), 7.06 (s, 1H), 6.85 (dd, J=2.0, 7.2 Hz, 1H), 6.59 (s, 1H), 4.09-4.01 (m, 1H), 4.00-3.94 (m, 1H), 3.91 (s, 3H), 3.48-3.37 (m, 2H), 2.85-2.76 (m, 1H), 2.61 (s, 3H), 2.45 (s, 3H), 2.11-1.94 (m, 2H)

### Example 295: (2*R, 3*S)—N-(2,6-dimethylpyrimidin-4-yl)-5-[2-methyl-4-[(2-methyltetrahydrofuran-3-yl)methoxy]pyrazol-3-yl]pyrazolo[1,5-a]pyridin-2-amine

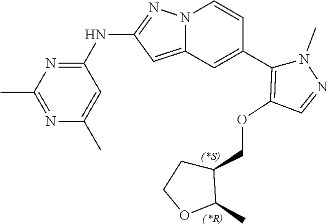

The title compound (23.9 mg, 2 steps, 3%) was prepared as described in Example 1 Steps A-B, except ((cis)-2-methyltetrahydrofuran-3-yl)methanol was used instead of (2s,4r)-tert-butyl 4-hydroxy-2-methylpyrrolidine-1-carboxylate in Step A and Intermediate 6 was used instead of 1-methyl-1h-pyrazol-4-ol in Step B. MS (ESI): mass calcd. for C₂₃H₂₇N₇O₂, 433.2. m/z found, 434.1 [M+H]⁺. ¹H NMR (400 MHz, DMSO-d6) δ 10.23 (s, 1H), 8.62 (d, J=7.2 Hz, 1H), 7.69 (d, J=0.8 Hz, 1H), 7.50 (s, 1H), 7.00 (s, 1H), 6.92 (dd, J=2.0, 7.2 Hz, 1H), 6.88 (s, 1H), 4.00-3.90 (m, 2H), 3.87 (s, 3H), 3.85-3.75 (m, 2H), 3.59-3.49 (m, 1H), 2.49-2.48 (m, 1H), 2.46 (s, 3H), 2.30 (s, 3H), 2.04-1.93 (m, 1H), 1.75-1.64 (m, 1H), 1.05 (d, J=6.4 Hz, 3H)

### Example 296: (2S)-1-[(2R)-2-[[1-methyl-5-[2-[(5-methylpyrazin-2-yl)amino]pyrazolo[1,5-a]pyridin-5-yl]pyrazol-4-yl]oxymethyl]azetidin-1-yl]propan-2-ol

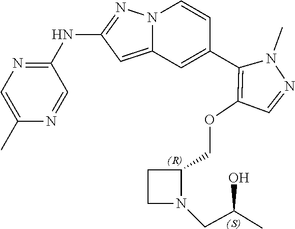

The title compound (41.3 mg, 18%) was prepared as described in Example 56 Step C, except (S)-2-methyloxirane was used instead of acetyl chloride and (R)-5-(4-(azetidin-2-ylmethoxy)-1-methyl-1H-pyrazol-5-yl)-N-(5-methylpyrazin-2-yl)pyrazolo[1,5-a]pyridin-2-amine was used instead of (R)—N-(5-(4-(azetidin-2-ylmethoxy)-1-methyl-1H-pyrazol-5-yl)pyrazolo[1,5-a]pyridin-2-yl)cyclopropanecarboxamide. MS (ESI): mass calcd. for C₂₃H₂₈N₈O₂, 448.2. m/z found, 449.1 [M+H]⁺. ¹H NMR (400 MHz, DMSO-d6) δ 10.08 (s, 1H), 8.59 (d, J=7.2 Hz, 1H), 8.56 (s, 1H), 8.10 (s, 1H), 7.66 (s, 1H), 7.45 (s, 1H), 6.91 (dd, J=1.6, 6.8 Hz, 1H), 6.87 (s, 1H), 4.23 (s, 1H), 4.06-3.91 (m, 2H), 3.86 (s, 3H), 3.60-3.47 (m, 1H), 3.44-3.36 (m, 2H), 2.90-2.73 (m, 1H), 2.57-2.54 (m, 1H), 2.38 (s, 3H), 2.30-2.18 (m, 1H), 2.07-1.81 (m, 2H), 0.95 (d, J=6.4 Hz, 3H)

### Example 297: (2S)-1-[(2R)-2-[[5-[2-[(5,6-dimethylpyrazin-2-yl)amino]pyrazolo[1,5-a]pyridin-5-yl]-1-methyl-pyrazol-4-yl]oxymethyl]azetidin-1-yl]propan-2-ol

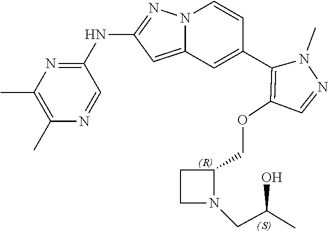

The title compound (184.5 mg, 61%) was prepared as described in Example 56 Step C, except (S)-2-methyloxirane was used instead of acetyl chloride and (R)-5-(4-(azetidin-2-ylmethoxy)-1-methyl-1H-pyrazol-5-yl)-N-(5,6-dimethylpyrazin-2-yl)pyrazolo[1,5-a]pyridin-2-amine was used instead of (R)—N-(5-(4-(azetidin-2-ylmethoxy)-1-methyl-1H-pyrazol-5-yl)pyrazolo[1,5-a]pyridin-2 yl)cyclopropanecarboxamide MS (ESI): mass calcd. for C₂₄H₃₀N₈O₂, 462.2; m/z found, 463.2 [M+H]⁺. ¹H NMR (400 MHz, CDCl₃) δ 8.46 (s, 1H), 8.36 (d, J=7.2 Hz, 1H), 7.44 (d, J=1.2 Hz, 1H), 7.33 (s, 1H), 7.31 (s, 1H), 6.80 (dd, J=1.6, 7.2 Hz, 1H), 6.68 (s, 1H), 4.01-3.92 (m, 2H), 3.90 (s, 3H), 3.65-3.52 (m, 2H), 3.49-3.41 (m, 1H), 3.25 (s, 1H), 2.89-2.77 (m, 1H), 2.52-2.44 (m, 7H), 2.40-2.33 (m, 1H), 2.12-1.93 (m, 2H), 1.05 (d, J=6.4 Hz, 3H)

### Example 298: N-(2,6-dimethylpyrimidin-4-yl)-5-[2-methyl-4-[(2S,3R)-2-methylpyrrolidin-3-yl]oxy-pyrazol-3-yl]pyrazolo[1,5-a]pyridin-2-amine

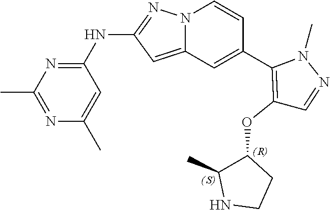

The title compound (57.3 mg, 3 steps, 5%) was prepared as described in Example 1 Steps A-B then Step E, except cis-tert-butyl 3-hydroxy-2-methylpyrrolidine-1-carboxylate was used instead of (2s,4r)-tert-butyl 4-hydroxy-2-methylpyrrolidine-1-carboxylate in Step A and Intermediate 10 was used instead of 1-methyl-1h-pyrazol-4-ol in Step B. After Step B the pure was further purified by SFC over DAICEL CHIRALCEL OD-H (250 mm×30 mm, 5 um), (eluent: 35% to 35% (v/v) supercritical CO₂ in EtOH and H₂O with 0.1% NH₃H₂O IPA) and the compound was first to elute at 5.56 min. The pure fractions were collected and the volatiles were removed under reduced pressure. The product was suspended in water (10 mL), the mixture frozen using dry ice/ethanol, and then lyophilized to dryness to afford the product, which was then carried to Step E. MS (ESI): mass calcd. for C₂₂H₂₆N₈O, 418.2. m/z found, 419.1 [M+H]⁺. ¹H NMR (400 MHz, CDCl₃) δ 8.39 (d, J=7.2 Hz, 1H), 7.47 (s, 1H), 7.45 (d, J=0.8 Hz, 1H), 7.30 (s, 1H), 7.05 (s, 1H), 6.83 (dd, J=2.0, 7.2 Hz, 1H), 6.62 (s, 1H), 4.21-4.16 (m, 1H), 3.91 (s, 3H), 3.35-3.26 (m, 1H), 3.05-2.99 (m, 2H), 2.61 (s, 3H), 2.45 (s, 3H), 2.09-1.88 (m, 2H), 1.13 (d, J=6.4 Hz, 3H)

### Example 299: (*S)—N-(2,6-dimethylpyrimidin-4-yl)-5-[4-(6,9-dioxaspiro[4.4]nonan-8-ylmethoxy)-2-methyl-pyrazol-3-yl]pyrazolo[1,5-a]pyridin-2-amine

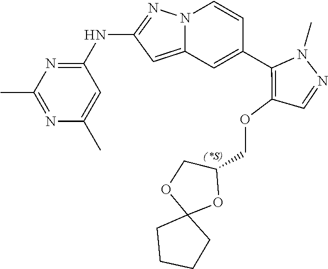

The title compound (56.2 mg, 2 steps, 9%) was prepared as described in Example 1 Steps A-B, except 1,4-dioxaspiro[4.4]nonan-2-ylmethanol was used instead of (2s,4r)-tert-butyl 4-hydroxy-2-methylpyrrolidine-1-carboxylate in Step A and Intermediate 10 was used instead of 1-methyl-1h-pyrazol-4-ol in Step B. After Step B the pure was further purified by SFC over DAICEL CHIRALCEL OJ-H(250 mm×30 mm, 5 um), (eluent: 45% to 45% (v/v) supercritical CO2 in EtOH and H₂O with 0.1% NH3H2O) and the title compound was second to elute. The pure fractions were collected and the volatiles were removed under reduced pressure. The product was suspended in water (10 mL), the mixture frozen using dry ice/ethanol, and then lyophilized to dryness to afford product. MS (ESI): mass calcd. for C₂₅H₂₉N₇O₃, 475.2. m/z found, 476.1 [M+H]⁺. ¹H NMR (400 MHz, DMSO-d6) δ 10.22 (s, 1H), 8.61 (d, J=7.6 Hz, 1H), 7.71 (d, J=1.2 Hz, 1H), 0.48 (s, 1H), 6.99 (s, 1H), 6.93 (dd, J=2.0, 7.2 Hz, 1H), 6.89 (s, 1H), 4.33-4.25 (m, 1H), 4.05-3.93 m, 3H), 3.86 (s, 3H), 3.70-3.64 (m, 1H), 2.46 (s, 3H), 2.30 (s, 3H), 1.75-1.52

### Example 300: (2R,3S)-2-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol

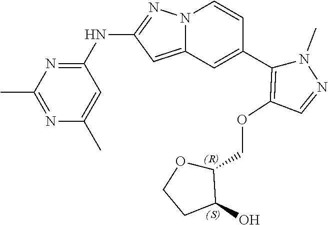

The title compound (55 mg, 2 steps, 20%) was prepared as described in Example 1 Steps A-B, except (2R,3S)-2-(hydroxymethyl)tetrahydrofuran-3-ol was used instead of (2s,4r)-tert-butyl 4-hydroxy-2-methylpyrrolidine-1-carboxylate in Step A and Intermediate 10 was used instead of 1-methyl-1h-pyrazol-4-ol in Step B. MS (ESI): mass calcd. for C₂₂H₂₅N₇O₃, 435.2. m/z found, 436.1 [M+H]⁺. ¹H NMR (400 MHz, CDCl₃) δ 8.38 (d, J=6.8 Hz, 1H), 7.52 (s, 1H), 7.45 (d, J=0.8 Hz, 1H), 7.37 (s, 1H), 7.04 (s, 1H), 6.82 (dd, J=2.0, 7.2 Hz, 1H), 6.60 (s, 1H), 4.39-4.34 (m, 1H), 4.09-3.93 (m, 5H), 3.90 (s, 3H), 2.60 (s, 3H), 2.44 (s, 3H), 2.19-2.09 (m, 1H), 1.94-1.86 (m, 1H)

### Example 301: (1S)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol

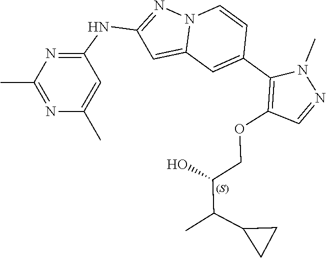

The title compound (84.2 mg, 2 steps, 18%) was prepared as described in Example 356. The title compound was second to elute at 3.23 min. MS (ESI): mass calcd. for C₂₃H₂₇N₇O₂, 433.2. m/z found, 434.2 [M+H]⁺. ¹H NMR (400 MHz, CDCl₃) δ 8.37 (d, J=7.2 Hz, 1H), 7.45 (d, J=0.8 Hz, 1H), 7.41-7.34 (m, 2H), 7.03 (s, 1H), 6.82 (dd, J=1.6, 6.8 Hz, 1H), 6.59 (s, 1H), 4.13-4.07 (m, 1H), 4.04-3.98 (m, 1H), 3.91 (s, 3H), 3.34-3.27 (m, 1H), 2.60 (s, 3H), 2.45 (s, 3H), 2.24 (br. s., 1H), 1.06 (s, 3H), 0.53-0.44 (m, 2H), 0.38-0.31 (m, 2H)

### Example 302: (S)-1-cyclobutyl-2-[5-[2-[(2,6-dimethylpyrimidin-4-yl)amino]pyrazolo[1,5-a]pyridin-5-yl]-1-methyl-pyrazol-4-yl]oxy-ethanol

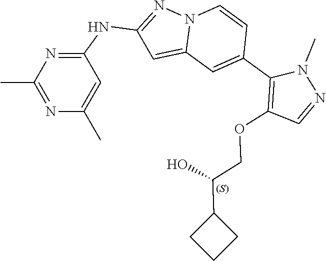

To a solution of 5-(2-((2,6-dimethylpyrimidin-4-yl)amino)pyrazolo[1,5-a]pyridin-5-yl)-1-methyl-1H-pyrazol-4-ol (400 mg, 1.19 mmol), 2-cyclobutyloxirane (234 mg, 2.39 mmol) and MeOH (15 mL) was added TEA (241 mg, 2.39 mmol). The reaction was stirred at 80° C. for 12 hours. The mixture was purified by preparative HPLC using a Phenomenex Gemini NX-C18 75×30 mm×3 um column (eluent: 27% to 57% (v/v) CH₃CN and H₂O with 0.05% NH₃) to afford pure product. The title compound was second to elute at 5.148 min. The product was further purified by SFC over Phenomenex-Cellulose-2 250 mm×30 mm, 10 μm (eluent: 50% to 50% (v/v) supercritical CO₂ in EtOH and H₂O with 0.1% NH₃). The pure fractions were collected and the volatiles were removed under reduced pressure. The product was suspended in water (10 mL), the mixture frozen using dry ice/acetone, and then lyophilized to dryness to afford the title compound as a white solid. MS (ESI): mass calcd. for C₂₃H₂₇N₇O₂, 433.2. m/z found, 434.1 [M+H]⁺. ¹H NMR (400 MHz, DMSO-d6) δ 10.22 (s, 1H), 8.61 (d, J=6.8 Hz, 1H), 7.71 (d, J=1.2 Hz, 1H), 7.42 (s, 1H), 7.04-6.94 (m, 2H), 6.88 (br. s., 1H), 4.84 (d, J=5.2 Hz, 1H), 3.87 (s, 3H), 3.80-3.71 (m, 2H), 3.68-3.58 (m, 1H), 2.46 (s, 3H), 2.40-2.34 (m, 1H), 2.30 (s, 3H), 1.93-1.76 (m, 5H), 1.74-1.65 (m, 1H)

### Example 303: (2S)-1-[(2R)-2-[[1-methyl-5-[2-[[2-methyl-6-(trifluoromethyl)pyrimidin-4-yl]amino]pyrazolo[1,5-a]pyridin-5-yl]pyrazol-4-yl]oxymethyl]azetidin-1-yl]propan-2-ol

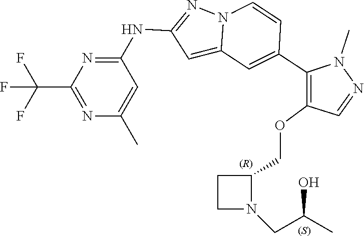

The title compound (55 mg, 3 steps, 9%) was prepared as described in Example 36 Step A then D-E, except 4-chloro-2-methyl-6-(trifluoromethyl)pyrimidine was used instead of methyl 6-bromonicotinate in Step A and (S)-2-methyloxirane was used instead of paraformaldehyde in Step E. MS (ESI): mass calcd. for C₂₄H₂₇F₃N₈O₂, 516.2. m/z found, 517.1 [M+H]⁺. ¹H NMR (400 MHz, CDCl₃) δ 8.43 (d, J=7.2 Hz, 1H), 8.01 (s, 1H), 7.57 (br. s., 1H), 7.51 (d, J=0.8 Hz, 1H), 7.34 (s, 1H), 6.91 (dd, J=1.6, 7.2 Hz, 1H), 6.65 (br. s., 1H), 4.02-3.93 (m, 2H), 3.92 (s, 3H), 3.66-3.55 (m, 2H), 3.50-3.42 (m, 1H), 3.27 (br. s., 1H), 2.89-2.79 (m, 1H), 2.70 (s, 3H), 2.53-2.44 (m, 1H), 2.41-2.34 (m, 1H), 2.13-1.95 (m, 2H), 1.05 (d, J=6.4 Hz, 3H) 19F NMR (376 MHz, CDCl3) δ −70.33 (s, 3F).

### Example 304: (*S)-2-cyclopropyl-1-[5-[2-[(2,6-dimethylpyrimidin-4-yl)amino]pyrazolo[1,5-a]pyridin-5-yl]-1-methyl-pyrazol-4-yl]oxy-propan-2-ol

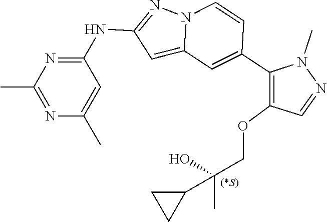 [SMILES: Cc1cc(Nc2cc3cc(-c4c(OC[C@](C)(O)C5CC5)cnn4C)ccn3n2)nc(C)n1]

The title compound (36.5 mg, 7%) was prepared as described in Example 1 Step B, except 2-cyclopropyl-2-methyloxirane and Intermediate 10 were used instead of (2S,4R)-2-methyl-4-(tosyloxy)pyrrolidine-1-carboxylate and 1-methyl-1h-pyrazol-4-ol in Step B. The compound was purified by SFC over Phenomenex-Cellulose-2 250 mm×30 mm, 10 μm (eluent: 50% to 50% (v/v) supercritical CO₂ in EtOH and H₂O with 0.1% NH₃). The title compound was second to elute at 2.681 min. The pure fractions were collected and the volatiles were removed under reduced pressure. The product was suspended in water (10 mL), the mixture frozen using dry ice/acetone, and then lyophilized to dryness to afford the title compound as a white solid. MS (ESI): mass calcd. for C₂₃H₂₇N₇O₂, 433.2. m/z found, 434.3 [M+H]⁺.

### Example 305: N-(2,6-dimethylpyrimidin-4-yl)-5-[4-[[(2R,4S)-1,4-dimethylpyrrolidin-2-yl]methoxy]-2-methyl-pyrazol-3-yl]pyrazolo[1,5-a]pyridin-2-amine

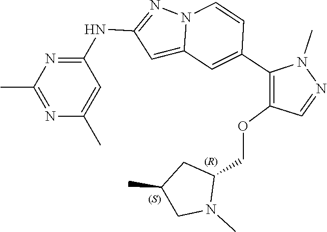

The title compound (51.2 mg, 4 steps, 0.9%) was prepared as described in Example 1 Steps A-B then Steps E-F, except (2R,4S)-tert-butyl 2-(hydroxymethyl)-4-methylpyrrolidine-1-carboxylate was used instead of (2s,4r)-tert-butyl 4-hydroxy-2-methylpyrrolidine-1-carboxylate in Step A and Intermediate 10 was used instead of 1-methyl-1h-pyrazol-4-ol in Step B. MS (ESI): mass calcd. for C₂₄H₃₀N₈O, 446.3. m/z found, 447.2 [M+H]⁺. ¹H NMR (400 MHz, CDCl₃) δ 8.37 (d, J=7.2 Hz, 1H), 7.47 (s, 1H), 7.37 (s, 1H), 7.35 (s, 1H), 7.06 (s, 1H), 6.86 (dd, J=2.0, 7.6 Hz, 1H), 6.58 (s, 1H), 4.03-3.90 (m, 4H), 3.89-3.81 (m, 1H), 3.20-3.07 (m, 1H), 2.76-2.67 (m, 1H), 2.61 (s, 3H), 2.45 (s, 3H), 2.40 (s, 3H), 2.31-2.18 (m, 1H), 1.97-1.90 (m, 1H), 1.87-1.79 (m, 1H), 1.59-1.49 (m, 1H), 0.99 (d, J=6.4 Hz, 3H)

### Example 306: (*S)-1-(3-bicyclo[1.1.1]pentanyl)-2-[5-[2-[(2,6-dimethylpyrimidin-4-yl)amino]pyrazolo[1,5-a]pyridin-5-yl]-1-methyl-pyrazol-4-yl]oxy-ethanol

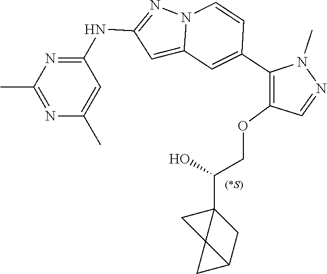 [SMILES: Cc1cc(Nc2cc3cc(-c4c(OC[C@@H](O)C56CC7CC75C6)cnn4C)ccn3n2)nc(C)n1]

Step A. 1-(bicyclo[1.1.1]pentan-1-yl)-2-((5-(2-((2,6-dimethylpyrimidin-4-yl)amino)pyrazolo[1,5-a]pyridin-5-yl)-1-methyl-1H-pyrazol-4-yl)oxy)ethan-1-one. The title compound (470 mg, 48%) was prepared as described in Example 1 Step B, except 1-(bicyclo[1.1.1]pentan-1-yl)-2-chloroethanone and Intermediate 10 were used instead of 1-methyl-1h-pyrazol-4-ol and (2S,4R)-2-methyl-4-(tosyloxy)pyrrolidine-1-carboxylate in Step B.

Step B. (*S)-1-(3-bicyclo[1.1.1]pentanyl)-2-[5-[2-[(2,6-dimethylpyrimidin-4-yl)amino]pyrazolo[1,5-a]pyridin-5-yl]-1-methyl-pyrazol-4-yl]oxy-ethanol. To a solution of 1-(bicyclo[1.1.1]pentan-1-yl)-2-((5-(2-((2,6-dimethylpyrimidin-4-yl)amino)pyrazolo[1,5-a]pyridin-5-yl)-1-methyl-1H-pyrazol-4-yl)oxy)ethanone (200 mg, 0.451 mmol) in MeOH (3 mL) was added NaBH₄ (34.1 mg, 0.902 mmol) at 0° C. The mixture was stirred at room temperature for 1 hr. The mixture was purified by Prep-HPLC using a Xtimate C18 150×40 mm×10 um column (eluent: 35% to 65% (v/v) CH₃CN and water (0.05% NH₃H₂O+10 mM NH₄HCO₃)-ACN) to afford pure product. The title compound was second to elute at 3.38 min. The product was suspended in water (10 mL), the mixture frozen using dry ice/ethanol, and then lyophilized to dryness to afford the title compound as a white solid. The compound was further purified by SFC (Column: DAICEL CHIRALCEL OJ-H(250 mm*30 mm, 5 um), Condition: 0.1% NH₃H₂O EtOH, Begin B: 30%, End B 30%. Flow Rate: 60 ml/min) to give the title compound (11.3 mg, 2 steps, 3%) as a white solid. MS (ESI): mass calcd. for C₂₄H₂₇N₇O₂, 445.2. m/z found, 446.1 [M+H]⁺. ¹H NMR (400 MHz, CDCl₃) δ 8.37 (d, J=7.2 Hz, 1H), 7.48 (br. s., 1H), 7.44 (s, 1H), 7.37 (s, 1H), 7.02 (s, 1H), 6.81 (d, J=7.2 Hz, 1H), 6.59 (s, 1H), 4.02-3.97 (m, 1H), 3.90 (s, 3H), 3.88-3.81 (m, 2H), 2.60 (s, 3H), 2.52 (s, 1H), 2.44 (s, 3H), 2.28 (br. s., 1H), 1.80-1.74 (m, 6H)

### Example 307: (*R)—N-(2,6-dimethylpyrimidin-4-yl)-5-[4-[(5,5-dimethyltetrahydrofuran-3-yl)methoxy]-2-methyl-pyrazol-3-yl]pyrazolo[1,5-a]pyridin-2-amine

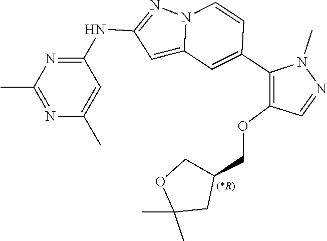 [SMILES: Cc1cc(Nc2cc3cc(-c4c(OC[C@@H]5COC(C)(C)C5)cnn4C)ccn3n2)nc(C)n1]

The title compound (35.6 mg, 2 steps, 11%) was prepared as described in Example 1 Steps A-B, except (5,5-dimethyltetrahydrofuran-3-yl)methanol was used instead of (2s,4r)-tert-butyl 4-hydroxy-2-methylpyrrolidine-1-carboxylate in Step A and Intermediate 10 was used instead of 1-methyl-1h-pyrazol-4-ol in Step B. The pure was further purified by SFC over DAICEL CHIRALPAK IG (250 mm×30 mm, 10 um), (eluent: 45% to 45% (v/v) supercritical CO₂ in EtOH and H₂O with 0.1% NH₃H₂O IPA). The title compound was first to elute at 2.58 min. The pure fractions were collected and the volatiles were removed under reduced pressure. The product was suspended in water (10 mL), the mixture frozen using dry ice/ethanol, and then lyophilized to dryness to afford the title compound as a white solid. MS (ESI): mass calcd. for C₂₄H₂₉N₇O₂, 447.2. m/z found, 448.1 [M+H]⁺. ¹H NMR (400 MHz, CDCl₃) δ 8.40 (d, J=7.2 Hz, 1H), 7.51 (s, 1H), 7.45 (d, J=0.8 Hz, 1H), 7.34 (s, 1H), 7.06 (s, 1H), 6.83 (dd, J=2.0, 7.2 Hz, 1H), 6.61 (s, 1H), 4.01-3.84 (m, 6H), 3.71-3.64 (m, 1H), 2.78 (m, 1H), 2.61 (s, 3H), 2.46 (s, 3H), 1.96-1.87 (m, 1H), 1.52-1.43 (m, 1H), 1.28 (s, 3H), 1.21 (s, 3H)

### Example 308: 5-[2-methyl-4-[[(2S)-1-methylazetidin-2-yl]methoxy]pyrazol-3-yl]-N-[6-methyl-2-(trifluoromethyl)pyrimidin-4-yl]pyrazolo[1,5-a]pyridin-2-amine

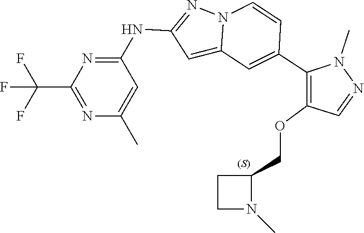

The title compound (55 mg, 3 steps, 30%) was prepared as described in Example 2 Steps A-C, except 4-chloro-6-methyl-2-(trifluoromethyl)pyrimidine was used instead of 4-chloro-2,6-dimethylpyrimidine in Step A. MS (ESI): mass calcd. for C₂₂H₂₃F₃N₈O, 472.2. m/z found, [M+H]⁺. ¹H NMR (400 MHz, CDCl₃) δ 8.46 (d, J=7.2 Hz, 1H), 8.12 (s, 1H), 7.51 (d, J=0.8 Hz, 1H), 7.41-7.30 (m, 2H), 6.91 (dd, J=2.0, 7.2 Hz, 1H), 6.63 (s, 1H), 4.00 (d, J=5.6 Hz, 2H), 3.93 (s, 3H), 3.48-3.41 (m, 1H), 3.40-3.31 (m, 1H), 2.90-2.79 (m, 1H), 2.57 (s, 3H), 2.34 (s, 3H), 2.09-1.96 (m, 2H) 19F NMR (376 MHz, CDCl3) δ −71.0 (s, 3F).

### Example 309: 5-[4-[2-amino-2-(1-methylcyclopropyl)ethoxy]-2-methyl-pyrazol-3-yl]-N-(2,6-dimethylpyrimidin-4-yl)pyrazolo[1,5-a]pyridin-2-amine

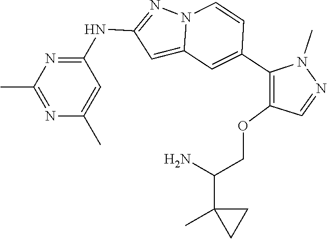 [SMILES: Cc1cc(Nc2cc3cc(-c4c(OCC(N)C5(C)CC5)cnn4C)ccn3n2)nc(C)n1]

Step A. 2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethan-1-one. The title compound (450 mg, 57%) was prepared as described in Example 1 Step B, except 2-chloro-1-(1-methylcyclopropyl)ethenone and Intermediate 10 were used instead of 1-methyl-1h-pyrazol-4-ol and (2S,4R)-2-methyl-4-(tosyloxy)pyrrolidine-1-carboxylate.

Step B. (Z)-2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethan-1-one oxime. To a solution of 2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethanone (100 mg, 0.232 mmol) in EtOH (2 mL) was added NH₂OH (50% in H₂O, 153 mg, 2.32 mmol). The mixture was stirred at 80° C. for 16 hour. The reaction mixture was poured into H₂O (3 mL). The mixture was extracted with ethyl acetate (5 mL×3). The combined organic layers were dried over Na₂SO₄, filtered and concentrated to give compound (110 mg, 106%, crude product) as yellow oil.

Step C. 5-[4-[2-amino-2-(1-methylcyclopropyl)ethoxy]-2-methyl-pyrazol-3-yl]-N-(2,6-dimethylpyrimidin-4-yl)pyrazolo[1,5-a]pyridin-2-amine. To a solution of (Z)-2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethanone oxime (110 mg, 0.246 mmol) in EtOH (4 mL) was added wet Raney Ni (20 mg, 50% in H₂O) and NH₃·H₂O (0.1 uL) to a hydrogenated bottle under N2 atmosphere. The suspension was degassed under vacuum and purged with N2 atmosphere for three times, and then purged with hydrogen for three times. The resulting mixture was stirred under H₂ (45 psi) at 25-30° C. for 16 hours. The mixture was purified by Prep-HPLC using a Phenomenex Gemini NX-C18 75×30 mm×3 um column (eluent: 25% to 55% (v/v) CH₃CN and water (0.05% NH₃H₂O+10 mM NH₄HCO₃)-ACN) to afford pure product. The product was suspended in water (10 mL), the mixture frozen using dry ice/ethanol, and then lyophilized to dryness to afford the title compound (10.8 mg, 9.4%) as a white solid. MS (ESI): mass calcd. for C₂₃H₂₈N₈O, 432.2. m/z found, 433.2 [M+H]⁺. ¹H NMR (400 MHz, CDCl₃) δ 8.38 (d, J=6.8 Hz, 1H), 7.56-7.41 (m, 2H), 7.37 (s, 1H), 7.06 (s, 1H), 6.84 (d, J=6.8 Hz, 1H), 6.60 (s, 1H), 4.11-4.02 (m, 1H), 3.99-3.84 (m, 4H), 2.61 (s, 3H), 2.51-2.38 (m, 4H), 1.03 (s, 3H), 0.45-0.28 (m, 4H)

### Example 310: 5-[4-[2-amino-2-(3-bicyclo[1.1.1]pentanyl)ethoxy]-2-methyl-pyrazol-3-yl]-N-(2,6-dimethylpyrimidin-4-yl)pyrazolo[1,5-a]pyridin-2-amine

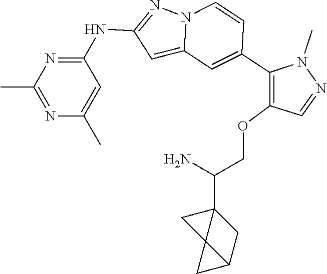 [SMILES: Cc1cc(Nc2cc3cc(-c4c(OCC(N)C56CC7CC75C6)cnn4C)ccn3n2)nc(C)n1]

The title compound (2.1 mg, 3 steps, 0.8%) was prepared as described in Example 309 Steps A-C, except 1-(bicyclo[1.1.1]pentan-1-yl)-2-chloroethanone was used instead of 2-chloro-1-(1-methylcyclopropyl)ethenone in Step A. MS (ESI): mass calcd. for C₂₄H₂₈N₈O, 444.2. m/z found, 445.2 [M+H]⁺. ¹H NMR (400 MHz, CDCl₃) δ 8.37 (d, J=7.2 Hz, 1H), 7.45 (s, 1H), 7.38-7.30 (m, 2H), 7.05 (s, 1H), 6.83 (d, J=6.8 Hz, 1H), 6.59 (s, 1H), 3.97-3.86 (m, 4H), 3.75-3.67 (m, 1H), 3.11-3.05 (m, 1H), 2.60 (s, 3H), 2.50 (s, 1H), 2.44 (s, 3H), 1.70-1.65 (m, 6H)

### Example 311: methyl 5-[[5-[4-[[(2R)-1-ethylazetidin-2-yl]methoxy]-2-methyl-pyrazol-3-yl]pyrazolo[1,5-a]pyridin-2-yl]amino]pyrazine-2-carboxylate

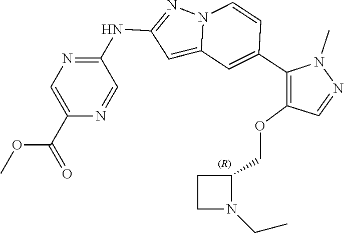

The title compound (614.2 mg, 3 steps, 11%) was prepared as described in Example 36 Step A then D-E, except methyl 5-chloropyrazine-2-carboxylate was used instead of methyl 6-bromonicotinate in Step A and acetaldehyde was used instead of paraformaldehyde in Step E. MS (ESI): mass calcd. for C₂₃H₂₆N₈O₃, 462.2. m/z found, 463.2 [M+H]⁺. ¹H NMR (400 MHz, CDCl₃) δ 8.98 (d, J=1.2 Hz, 1H), 8.71 (d, J=1.2 Hz, 1H), 8.37 (d, J=7.2 Hz, 1H), 8.02 (s, 1H), 7.51 (d, J=0.8 Hz, 1H), 7.35 (s, 1H), 6.89 (dd, J=2.0, 7.2 Hz, 1H), 6.84 (s, 1H), 4.13-4.06 (m, 1H), 4.03-3.98 (m, 4H), 3.91 (s, 3H), 3.54-3.42 (m, 2H), 2.91-2.80 (m, 1H), 2.77-2.67 (m, 1H), 2.47-2.35 (m, 1H), 2.13-2.00 (m, 2H), 0.97 (t, J=7.2 Hz, 3H)

### Example 312: N-[5-[2-ethyl-4-[[(3R)-pyrrolidin-3-yl]methoxy]pyrazol-3-yl]pyrazolo[1,5-a]pyridin-2-yl]cyclopropanecarboxamide

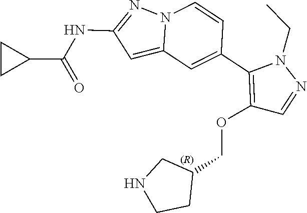

The title compound (58.3 mg, 3 steps, 13%) was prepared as described in Example 1 Steps A-B then Step E, except (R)-tert-butyl 3-(hydroxymethyl)pyrrolidine-1-carboxylate was used instead of (2s,4r)-tert-butyl 4-hydroxy-2-methylpyrrolidine-1-carboxylate in Step A and N-(5-(1-ethyl-4-hydroxy-1H-pyrazol-5-yl)pyrazolo[1,5-a]pyridin-2-yl)cyclopropanecarboxamide was used instead of 1-methyl-1h-pyrazol-4-ol in Step B. The material N-(5-(1-ethyl-4-hydroxy-1H-pyrazol-5-yl)pyrazolo[1,5-a]pyridin-2-yl)cyclopropanecarboxamide was prepared as described in Intermediate 4, except 5-bromo-1-ethyl-1H-pyrazol-4-ol was used instead of 5-bromo-1-methyl-1H-pyrazol-4-ol. MS (ESI): mass calcd. for C₂₁H₂₆N₆O₂, 394.2. m/z found, 395.2 [M+H]⁺. ¹H NMR (400 MHz, DMSO-d6) δ 11.11 (s, 1H), 8.60 (d, J=6.8 Hz, 1H), 7.63 (s, 1H), 7.54-7.44 (m, 1H), 6.91 (s, 1H), 6.89-6.85 (m, 1H), 4.13 (q, J=7.2 Hz, 2H), 3.95-3.76 (m, 2H), 3.34-3.28 (m, 1H), 3.23-3.18 (m, 0.5H), 3.06-2.99 (m, 0.5H), 2.87-2.74 (m, 1.3H), 2.72-2.64 (m, 0.7H), 2.57-2.54 (m, 0.5H), 2.43-2.29 (m, 0.5H), 1.97-1.85 (m, 1.3H), 1.81-1.70 (m, 0.7H), 1.68-1.55 (m, 0.5H), 1.38-1.32 (m, 0.5H), 1.26 (t, J=7.2 Hz, 3H), 0.89-0.74 (m, 4H)

### Example 313: N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide

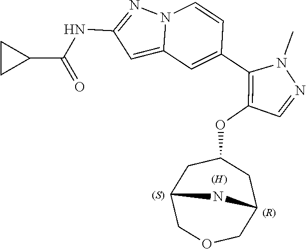

Step A. Tert-butyl (1R,5S,7r)-7-((4-nitrobenzoyl)oxy)-3-oxa-9-azabicyclo[3.3.1]nonane-9-carboxylate. To a round bottom flask containing a stir bar was added TPP (1078.042 mg, 4.11 mmol), endo-tert-butyl (1R,5S,7s)-7-hydroxy-3-oxa-9-azabicyclo[3.3.1]nonane-9-carboxylate (500 mg, 2.055 mmol), 4-nitrobenzoic acid (343.442 mg, 2.055 mmol), THE (10.275 mL, 0.2 M, 2.055 mmol) and then dropwise addition of DIAD (0.809 mL, 1.027 g/mL, 4.11 mmol) in 2 mL of THE by syringe under N₂. The reaction was stirred for 3 h at room temperature. The reaction was stirred overnight at 40 C. The crude was diluted with EtOAc and washed with brine 1×. The pure fraction was collected and concentrated down under vacuo to obtain a yellow solid (450 mg, 56%).

Step B. Tert-butyl (1R,5S,7r)-7-hydroxy-3-oxa-9-azabicyclo[3.3.1]nonane-9-carboxylate. To a mixture of tert-butyl (1R,5S,7r)-7-((4-nitrobenzoyl)oxy)-3-oxa-9-azabicyclo[3.3.1]nonane-9-carboxylate (1360 mg, 3.466 mmol), THE (12.65 mL, 0.889 g/mL, 155.96 mmol), and water, distilled (3.066 mL, 0.998 g/mL, 169.823 mmol) was added LiOH (124.499 mg, 5.199 mmol). The reaction was stirred for 2 h at room temperature. The crude material was diluted with EtOAc. The water layer was separated. The organic layer was dried over anhydrous MgSO4, filtered, and concentrated down. The compound solidified to a white solid after high vacuum. The crude was flashed with 30-100% EtOAc:hex. The compound (480 mg, 57%) was isolated based on ELSD on ISCO, confirmed by LCMS.

Step C. Tert-butyl (1S,5S)-7-((5-(2-(cyclopropanecarboxamido)pyrazolo[1,5-a]pyridin-5-yl)-1-methyl-1H-pyrazol-4-yl)oxy)-3-oxa-9-azabicyclo[3.3.1]nonane-9-carboxylate. To a 100 mL round bottom flask charged with TPP (104.099 mg, 0.397 mmol), EXO-tert-butyl (1R,5S,7r)-7-hydroxy-3-oxa-9-azabicyclo[3.3.1]nonane-9-carboxylate (96.563 mg, 0.397 mmol) and N-(5-(4-hydroxy-1-methyl-1H-pyrazol-5-yl)pyrazolo[1,5-a]pyridin-2-yl)cyclopropanecarboxamide (59 mg, 0.198 mmol) was added THE (0.992 mL, 0.2 M, 0.198 mmol). The solid was allowed to dissolved and then cooled to 0 C for 5 min. To the resulting solution was added DIAD (0.0781 mL, 1.027 g/mL, 0.397 mmol) in slow drops. After addition of the DIAD, the ice-bath was removed and the solution was heated to 60° C. for 2 h. The reaction was heated for 1.5 h more then worked up to confirm product formation by quenching with 0.2 mL of H₂O and then concentrated down under vacuo. The crude was flashed with 30-100% EtOAc:hex. The fractions were collected and further purified via ISCO preparative HPLC using 10-100% ACN-H₂O with 0.05% TFA as modifier. The pure fractions were contaminated with PPhO. This material was taken onto next step without further purification.

Step D. N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide. The title compound (6.5 mg, 42%) was prepared as described in Example 1 Step F, except tert-butyl (1S,5S)-7-((5-(2-(cyclopropanecarboxamido)pyrazolo[1,5-a]pyridin-5-yl)-1-methyl-1H-pyrazol-4-yl)oxy)-3-oxa-9-azabicyclo[3.3.1]nonane-9-carboxylate was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₂H₂₆N₆O₃, 422.2. m/z found, 423.2 [M+H]⁺. ¹H NMR (500 MHz, Methanol-d4) δ 8.45 (dt, J=7.3, 1.0 Hz, 1H), 7.62 (dd, J=2.0, 1.0 Hz, 1H), 7.42 (s, 1H), 6.95 (dd, J=7.2, 2.0 Hz, 1H), 6.91 (s, 1H), 5.11-4.99 (m, 1H), 3.86 (s, 3H), 3.80-3.69 (m, 4H), 3.07 (s, 2H), 2.29 (dd, J=13.1, 6.2 Hz, 2H), 1.87 (dt, J=8.0, 3.7 Hz, 1H), 1.80 (td, J=11.6, 4.9 Hz, 2H), 1.29 (s, 1H), 1.00 (dt, J=4.6, 3.2 Hz, 2H), 0.91 (dt, J=8.1, 3.3 Hz, 3H).

### Example 314: N-[5-[4-[(1,3-dimethylazetidin-3-yl)methoxy]-2-methyl-pyrazol-3-yl]pyrazolo[1,5-a]pyridin-2-yl]cyclopropanecarboxamide

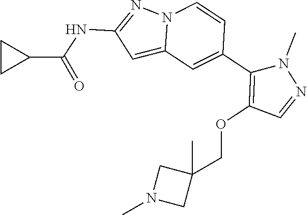 [SMILES: CN1CC(C)(COc2cnn(C)c2-c2ccn3nc(NC(=O)C4CC4)cc3c2)C1]

The title compound (29.6 mg, 4 steps, 0.9%) was prepared as described in Example 1 Steps A-B then Steps E-F, except tert-butyl 3-(hydroxymethyl)-3-methylazetidine-1-carboxylate was used instead of (2s,4r)-tert-butyl 4-hydroxy-2-methylpyrrolidine-1-carboxylate in Step A and Intermediate 4 was used instead of 1-methyl-1h-pyrazol-4-ol in Step B. MS (ESI): mass calcd. for C₂₁H₂₆N₆O₂, 394.2. m/z found, 395.3 [M+H]⁺. ¹H NMR (400 MHz, DMSO-d6) δ 11.11 (s, 1H), 8.61 (d, J=7.2 Hz, 1H), 7.71 (s, 1H), 7.47 (s, 1H), 6.97 (dd, J=1.2, 7.6 Hz, 1H), 6.89 (s, 1H), 3.91 (s, 2H), 3.87 (s, 3H), 3.11 (d, J=7.2 Hz, 2H), 2.86 (d, J=7.2 Hz, 2H), 2.20 (s, 3H), 1.99-1.87 (m, 1H), 1.22 (s, 3H), 0.85-0.78 (m, 4H)

### Example 315: N-[5-[2-methyl-4-[[(2R)-1-(2,2,2-trifluoroethyl)azetidin-2-yl]methoxy]pyrazol-3-yl]pyrazolo[1,5-a]pyridin-2-yl]cyclopropanecarboxamide

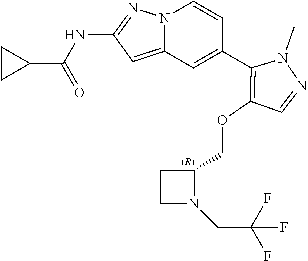

A solution of (R)—N-(5-(4-(azetidin-2-ylmethoxy)-1-methyl-1H-pyrazol-5-yl)pyrazolo[1,5-a]pyridin-2-yl)cyclopropanecarboxamide (Example 346, 100 mg, 0.273 mmol), 2,2,2-trifluoroethyl trifluoromethanesulfonate (63.3 mg, 0.273 mmol), DIEA (106 mg, 0.819 mmol) and DMF (3 mL) was stirred at 20° C. for 16 hours. The reaction mixture was then filtered and the filtrate was purified by preparative HPLC using a YMC-Triart Prep C18 250×50 mm×10 um column (eluent: 32% to 62% (v/v) CH₃CN and H₂O with 0.04% NH₃) to afford pure product. The product was suspended in water (10 mL), the mixture frozen using dry ice/ethanol, and then lyophilized to dryness to afford the title compound (6.7 mg, 5.5%) as a yellow oil. MS (ESI): mass calcd. for C₂₁H₂₃F₃N₆O₂, 448.2. m/z found, 449.2 [M+H]⁺. ¹H NMR (400 MHz, CDCl₃) δ 8.40 (s, 1H), 8.29 (d, J=7.2 Hz, 1H), 7.45 (s, 1H), 7.33 (s, 1H), 7.00 (s, 1H), 6.82 (d, J=6.8 Hz, 1H), 4.04-3.94 (m, 2H), 3.90 (s, 3H), 3.71-3.52 (m, 2H), 3.22-3.03 (m, 2H), 3.00-2.86 (m, 1H), 2.13-2.03 (m, 2H), 1.62-1.54 (m, 1H), 1.20-1.11 (m, 2H), 0.96-0.87 (m, 2H) 19F NMR (376 MHz, CDCl3) δ −70.86-−70.99 (m, 3F)

### Example 316: N-[5-[5-methyl-3-(4-piperidyloxy)isoxazol-4-yl]pyrazolo[1,5-a]pyridin-2-yl]cyclopropanecarboxamide

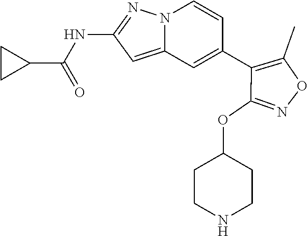 [SMILES: Cc1onc(OC2CCNCC2)c1-c1ccn2nc(NC(=O)C3CC3)cc2c1]

The title compound (73 mg, 3 steps, 15%) was prepared as described in Example 31 where 1-boc-4-methanesulfonyloxypiperidine was used instead of (S)-2-((tert-butoxycarbonyl)amino)-2-phenylethyl 4-methylbenzenesulfonate. MS (ESI): mass calcd. for C₂₀H₂₃N₅O₃, 381.2. m/z found, 382.2 [M+H]⁺. ¹H NMR (500 MHz, DMSO-d6) δ 11.04 (s, 1H), 8.57 (dd, J=7.2, 0.9 Hz, 1H), 7.64 (dd, J=2.1, 0.9 Hz, 1H), 6.90 (dd, J=7.2, 2.0 Hz, 1H), 6.85 (s, 1H), 4.79-4.67 (m, 1H), 2.99-2.86 (m, 2H), 2.60-2.53 (m, 2H), 2.51 (s, 3H), 2.04-1.96 (m, 2H), 1.96-1.88 (m, 1H), 1.65-1.52 (m, 2H), 0.90-0.76 (m, 4H).

### Example 317: *S, *R—N-[5-[3-(4-methoxy-1-methyl-pyrrolidin-3-yl)oxy-5-methyl-isoxazol-4-yl]pyrazolo[1,5-a]pyridin-2-yl]cyclopropanecarboxamide

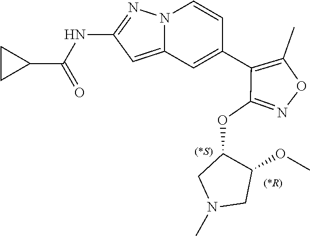 [SMILES: CO[C@@H]1CN(C)C[C@@H]1Oc1noc(C)c1-c1ccn2nc(NC(=O)C3CC3)cc2c1]

The amine precursor was prepared as described in Example 31 (80.3 mg, 3 steps, 6.7% yield) where tert-butyl (3R,4R)-3-methoxy-4-((methylsulfonyl)oxy)pyrrolidine-1-carboxylate was used instead of (S)-2-((tert-butoxycarbonyl)amino)-2-phenylethyl 4-methylbenzenesulfonate. The title compound (22.5 mg, 62%) was prepared as described in Example 29 where tert-butyl (3S,4R)-3-((4-(2-(cyclopropanecarboxamido)pyrazolo[1,5-a]pyridin-5-yl)-5-methylisoxazol-3-yl)oxy)-4-methoxypyrrolidine-1-carboxylate was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. The racemic mixture was separated using chiral preparative SFC using AD-H (3×25 cm) as immobile phase and 45% EtOH (0.1% DEA)/CO₂, 100 bar at 60 mL/min as mobile phase. MS (ESI): mass calcd. for C₂₁H₂₅N₅O₄, 411.2. m/z found, 412.2 [M+H]⁺. ¹H NMR (500 MHz, DMSO-d6) δ 11.05 (s, 1H), 8.58 (dd, J=7.3, 1.0 Hz, 1H), 7.68 (dd, J=2.1, 0.9 Hz, 1H), 6.96 (dd, J=7.3, 2.0 Hz, 1H), 6.83 (s, 1H), 5.16-5.08 (m, 1H), 4.06-3.95 (m, 1H), 3.25 (s, 3H), 3.08 (dd, J=10.6, 5.9 Hz, 1H), 2.89 (dd, J=9.5, 6.3 Hz, 1H), 2.61 (dd, J=10.7, 4.3 Hz, 1H), 2.53 (s, 3H), 2.25 (s, 3H), 1.98-1.86 (m, 1H), 0.90-0.75 (m, 4H).

### Example 318: 5-[3-[(3-methoxyazetidin-3-yl)methoxy]-5-methyl-isoxazol-4-yl]-N-pyrazin-2-yl-pyrazolo[1,5-a]pyridin-2-amine

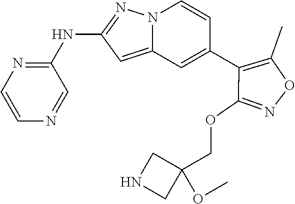 [SMILES: COC1(COc2noc(C)c2-c2ccn3nc(Nc4cnccn4)cc3c2)CNC1]

The title compound (13.7 mg, 4 steps, 16.5%) was prepared as described in Example 30 where 2-bromopyrazine was used instead of 4-chloro-2,6-dimethylpyrimidine. MS (ESI): mass calcd. for C₂₀H₂₁N₇O₃, 407.2. m/z found, 408.2 [M+H]⁺. ¹H NMR (400 MHz, DMSO-d6) δ 10.25 (s, 1H), 8.62 (s, 1H), 8.59 (d, J=7.3 Hz, 1H), 8.24-8.17 (m, 1H), 8.01 (d, J=2.8 Hz, 1H), 7.63 (d, J=1.9 Hz, 1H), 6.87 (q, J=2.9, 1.8 Hz, 2H), 4.57 (s, 2H), 3.53 (d, J=8.5 Hz, 2H), 3.29-3.25 (m, 2H), 3.20 (s, 3H), 2.54 (s, 3H).

### Example 319: N-[5-[3-[(3-aminocyclobutyl)methoxy]-5-methyl-isoxazol-4-yl]pyrazolo[1,5-a]pyridin-2-yl]cyclopropanecarboxamide

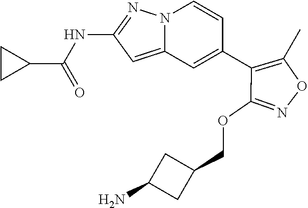 [SMILES: Cc1onc(OC[C@H]2C[C@@H](N)C2)c1-c1ccn2nc(NC(=O)C3CC3)cc2c1]

The title compound (58 mg, 3 steps, 12%) was prepared as described in Example 31 where tert-butyl ((cis)-3-(((4-bromo-5-methylisoxazol-3-yl)oxy)methyl)cyclobutyl)carbamate was used instead of (S)-2-((tert-butoxycarbonyl)amino)-2-phenylethyl 4-methylbenzenesulfonate. MS (ESI): mass calcd. for C₂₀H₂₃N₅O₃, 381.2. m/z found, 382.2 [M+H]⁺. ¹H NMR (400 MHz, DMSO-d6) δ 11.08 (s, 1H), 8.58 (d, J=7.2 Hz, 1H), 7.67-7.59 (m, 1H), 6.94-6.88 (m, 1H), 6.85 (s, 1H), 4.20 (d, J=4.8 Hz, 2H), 3.20-3.15 (m, 1H), 2.52 (s, 3H), 2.33-2.23 (m, 3H), 1.99-1.87 (m, 1H), 1.57-1.41 (m, 2H), 0.89-0.77 (m, 4H) 1H NMR (400 MHz, CDCl₃) δ 8.48 (s, 1H), 8.27 (d, J=6.8 Hz, 1H), 7.47 (s, 1H), 6.96 (s, 1H), 6.82 (dd, J=1.6, 7.6 Hz, 1H), 4.27 (d, J=6.0 Hz, 2H), 3.41-3.31 (m, 1H), 2.52 (s, 3H), 2.50-2.44 (m, 2H), 2.42-2.33 (m, 1H), 1.59-1.54 (m, 3H), 1.18-1.11 (m, 2H), 0.94-0.87 (m, 2H)

### Example 320: 5-[3-[[(2R)-azetidin-2-yl]methoxy]-5-methyl-isoxazol-4-yl]-N-(2,6-dimethylpyrimidin-4-yl)pyrazolo[1,5-a]pyridin-2-amine

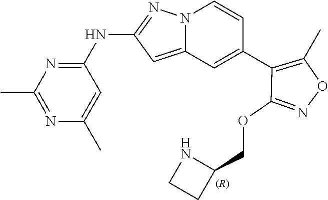

The title compound (37 mg, 4 steps, 11.9%) was prepared as described in Example 30, with a slight modification where step C was performed at the last step, where tert-butyl (R)-2-((tosyloxy)methyl)azetidine-1-carboxylate was used instead of tert-butyl 3-methoxy-3-((tosyloxy)methyl)azetidine-1-carboxylate. MS (ESI): mass calcd. for C₂₁H₂₃N₇O₂, 405.2. m/z found, 406.2 [M+H]⁺. ¹H NMR (400 MHz, DMSO-d6) δ 10.17 (s, 1H), 8.58 (d, J=7.2 Hz, 1H), 7.66 (s, 1H), 7.00 (s, 1H), 6.91 (dd, J=7.2, 2.0 Hz, 1H), 6.83 (s, 1H), 4.27 (d, J=5.2 Hz, 2H), 4.17 (s, 1H), 3.50 (t, J=7.9 Hz, 1H), 2.53 (s, 3H), 2.46 (s, 3H), 2.31-2.25 (m, 4H), 2.18-2.10 (m, 1H).

### Example 321: N-[5-[5-methyl-3-[[(2S)-1,3,3-trimethylazetidin-2-yl]methoxy]isoxazol-4-yl]pyrazolo[1,5-a]pyridin-2-yl]cyclopropanecarboxamide

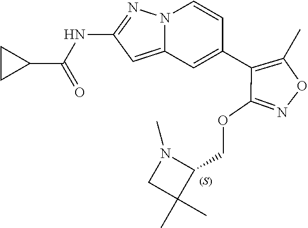

The amine precursor was prepared as described in Example 31 (79 mg, 3 steps, 26% yield) where tert-butyl (S)-2-(((4-bromo-5-methylisoxazol-3-yl)oxy)methyl)-3,3-dimethylazetidine-1-carboxylate was used instead of tert-butyl (S)-(2-((4-(2-(cyclopropanecarboxamido)pyrazolo[1,5-a]pyridin-5-yl)-5-methylisoxazol-3-yl)oxy)-1-phenylethyl)carbamate. The title compound (43 mg, 64%) was prepared as described in Example 29 where (S)—N-(5-(3-((3,3-dimethylazetidin-2-yl)methoxy)-5-methylisoxazol-4-yl)pyrazolo[1,5-a]pyridin-2-yl)cyclopropanecarboxamide was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. MS (ESI): mass calcd. for C₂₂H₂₇N₅O₃, 409.2. m/z found, 410.3 [M+H]⁺. ¹H NMR (400 MHz, DMSO-d6) δ 11.05 (s, 1H), 8.57 (dd, J=7.2, 0.9 Hz, 1H), 7.62 (dd, J=2.0, 0.9 Hz, 1H), 6.87 (dd, J=7.2, 2.0 Hz, 1H), 6.81 (d, J=0.9 Hz, 1H), 4.32-4.19 (m, 2H), 3.08-2.98 (m, 2H), 2.56-2.52 (m, 4H), 2.25 (s, 3H), 1.98-1.87 (m, 1H), 1.16 (s, 3H), 1.09 (s, 3H), 0.86-0.77 (m, 4H).

### Example 322: 5-[5-methyl-3-[(3S,5R)-5-methylpyrrolidin-3-yl]oxy-isoxazol-4-yl]-N-(5-methylpyrazin-2-yl)pyrazolo[1,5-a]pyridin-2-amine

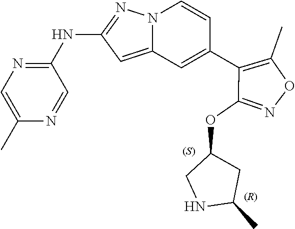

The title compound (58 mg, 4 steps, 35%) was prepared as described in Example 320, where tert-butyl (2R,4R)-2-methyl-4-(tosyloxy)pyrrolidine-1-carboxylate was used instead of tert-butyl (R)-2-((tosyloxy)methyl)azetidine-1-carboxylate. MS (ESI): mass calcd. for C₂₁H₂₃N₇O₂, 405.2. m/z found, 406.2 [M+H]⁺. ¹H NMR (400 MHz, DMSO-d6) δ 10.03 (s, 1H), 8.58-8.53 (m, 2H), 8.10 (t, J=1.1 Hz, 1H), 7.63 (dd, J=2.0, 0.9 Hz, 1H), 6.86 (dd, J=7.2, 2.0 Hz, 1H), 6.82 (d, J=0.8 Hz, 1H), 5.15-5.06 (m, 1H), 3.12 (d, J=12.7 Hz, 1H), 3.06-2.98 (m, 1H), 2.94 (dd, J=12.7, 5.4 Hz, 1H), 2.52 (s, 3H), 2.41-2.34 (m, 4H), 1.46-1.35 (m, 1H), 1.13 (d, J=6.3 Hz, 3H).

### Example 323: 5-[5-methyl-3-[[(2R)-1-methylazetidin-2-yl]methoxy]isoxazol-4-yl]-N-(5-methylpyrazin-2-yl)pyrazolo[1,5-a]pyridin-2-amine

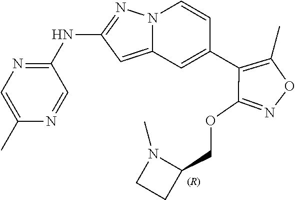

The amine precursor was prepared as described in Example 320 (43 mg, 3 steps, 21% yield) where tert-butyl (R)-2-((tosyloxy)methyl)azetidine-1-carboxylate was used instead of (S)-2-((tert-butoxycarbonyl)amino)-2-phenylethyl 4-methylbenzenesulfonate. The title compound (21 mg, 67%) was prepared as described in Example 29 where (R)-5-(3-(azetidin-2-ylmethoxy)-5-methylisoxazol-4-yl)-N-(5-methylpyrazin-2-yl)pyrazolo[1,5-a]pyridin-2-amine was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. MS (ESI): mass calcd. for C₂₁H₂₃N₇O₂, 405.2. m/z found, 406.2 [M+H]⁺. ¹H NMR (500 MHz, DMSO-d6) δ 10.03 (s, 1H), 8.58 (dt, J=7.3, 0.9 Hz, 1H), 8.56 (d, J=1.5 Hz, 1H), 8.10 (dd, J=1.6, 0.8 Hz, 1H), 7.62 (dd, J=2.0, 0.9 Hz, 1H), 6.86 (dd, J=7.2, 2.0 Hz, 1H), 6.79 (d, J=0.8 Hz, 1H), 4.28 (dd, J=10.6, 4.0 Hz, 1H), 4.21 (dd, J=10.7, 6.5 Hz, 1H), 3.40-3.34 (m, 1H), 3.29-3.24 (m, 1H), 2.79-2.70 (m, 1H), 2.53 (s, 3H), 2.38 (s, 3H), 2.24 (s, 3H), 2.04-1.87 (m, 2H).

### Example 324: N-[5-[3-[(2S)-2-(dimethylamino)-2-(2-fluorophenyl)ethoxy]-5-methyl-isoxazol-4-yl]pyrazolo[1,5-a]pyridin-2-yl]cyclopropanecarboxamide

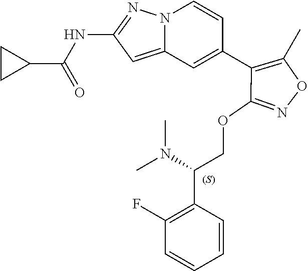

Step A. tert-butyl (S)-(2-((4-bromo-5-methylisoxazol-3-yl)oxy)-1-(2 fluorophenyl)ethyl)carbamate. DIAD (256 mg, 1.47 mmol) was added to a solution consisting of (S)-tert-butyl (1-(2-fluorophenyl)-2-hydroxyethyl)carbamate (250 mg, 0.979 mmol), 4-bromo-5-methylisoxazol-3-ol (209 mg, 1.18 mmol), PPh₃ (514 mg, 1.96 mmol), and anhydrous THE (15 mL). The resultant mixture was stirred at 70° C. for 12 hours. The reaction mixture was concentrated to dryness under reduced pressure to afford the crude product, which was purified by FCC (eluent: DCM:MeOH=1:0 to 10:1) to afford the title compound (200 mg, 49%) as a yellow solid. MS (ESI): mass calcd. for C17H20BrFN2O4, 415.1. m/z found, 415.0 [M+H]⁺.

Step B-D. N-[5-[3-[(2S)-2-(dimethylamino)-2-(2-fluorophenyl)ethoxy]-5-methyl-isoxazol-4-yl]pyrazolo[1,5-a]pyridin-2-yl]cyclopropanecarboxamide. The title compound (27 mg, 3 steps, 50%) was prepared as described in Example 31 and Example 29, where (S)-tert-butyl (2-((4-bromo-5-methylisoxazol-3-yl)oxy)-1-(2-fluorophenyl)ethyl)carbamate was used instead of tert-butyl (S)-(2-((4-bromo-5-methylisoxazol-3-yl)oxy)-1-phenylethyl)carbamate. MS (ESI): mass calcd. for C₂₅H₂₆FN₅O₃, 463.2. m/z found, 464.1 [M+H]⁺. 1H NMR (400 MHz, DMSO-d6) δ 7.75 (d, J=8.8 Hz, 1H), 7.54-7.44 (m, 1H), 7.40-7.30 (m, 1H), 7.26-7.13 (m, 2H), 5.43-5.20 (m, 1H), 4.40-4.20 (m, 2H), 2.33 (s, 3H), 1.37 (s, 9H)

### Example 325: N-[5-[3-[(2S)-2-(dimethylamino)-2-(2-pyridyl)ethoxy]-5-methyl-isoxazol-4-yl]pyrazolo[1,5-a]pyridin-2-yl]cyclopropanecarboxamide

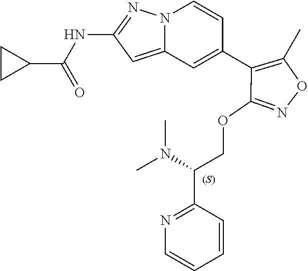

The title compound (16.1 mg, 4 steps, 2.65%) was prepared as described in Example 324Example 320, where (S)-tert-butyl (2-hydroxy-1-(pyridin-2-yl)ethyl)carbamate was used instead of (S)-tert-butyl (1-(2-fluorophenyl)-2-hydroxyethyl)carbamate. MS (ESI): mass calcd. for C₂₄H₂₆N₆O₃, 446.2. m/z found, 447.1 [M+H]⁺. ¹H NMR (400 MHz, CD3OD) δ 8.57 (d, J=4.4 Hz, 1H), 8.27 (d, J=7.2 Hz, 1H), 7.90-7.79 (m, 1H), 7.53 (d, J=7.6 Hz, 1H), 7.43-7.33 (m, 1H), 7.21 (s, 1H), 6.76 (s, 1H), 6.67-6.57 (m, 1H), 4.81-4.74 (m, 2H), 3.99 (t, J=6.4 Hz, 1H), 2.48 (s, 3H), 2.33 (s, 6H), 1.93-1.81 (m, 1H), 1.05-0.98 (m, 2H), 0.94-0.85 (m, 2H)

### Example 326: N-[5-[3-[(2R)-2-amino-2-phenyl-propoxy]-5-methyl-isoxazol-4-yl]pyrazolo[1,5-a]pyridin-2-yl]cyclopropanecarboxamide

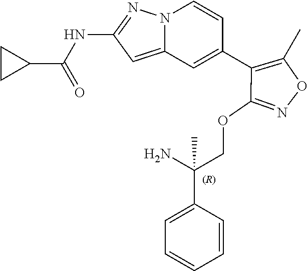

The title compound (4.5 mg, 3 steps, 4%) was prepared as described in Example 31 where (R)-tert-butyl 4-methyl-4-phenyl-1,2,3-oxathiazolidine-3-carboxylate 2,2-dioxide was used instead of (S)-2-((tert-butoxycarbonyl)amino)-2-phenylethyl 4-methylbenzenesulfonate. MS (ESI): mass calcd. for C₂₄H₂₅N₅O₃, 431.2. m/z found, 432.1 [M+H]⁺. ¹H NMR (400 MHz, CD3OD) δ 8.35 (d, J=7.2 Hz, 1H), 7.58-7.53 (m, 4H), 7.53-7.48 (m, 1H), 7.32 (s, 1H), 6.73 (d, J=6.4 Hz, 1H), 4.80-4.65 (m, 2H), 2.51 (s, 3H), 1.89 (br s, 1H), 1.86 (s, 3H), 1.06-0.99 (m, 2H), 0.96-0.89 (m, 2H)

### Example 327: 5-[3-[(3S,5S)-5-(difluoromethyl)pyrrolidin-3-yl]oxy-5-methyl-isoxazol-4-yl]-N-(2,6-dimethylpyrimidin-4-yl)pyrazolo[1,5-a]pyridin-2-amine

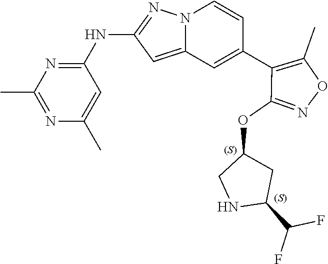

Step A. tert-butyl (2S,4S)-4-((4-bromo-5-methylisoxazol-3-yl)oxy)-2-(difluoromethyl)pyrrolidine-1-carboxylate. The titled prepared (1430 mg, 85%) as described in Example 324 step A using tert-butyl (2S,4R)-2-(difluoromethyl)-4-hydroxypyrrolidine-1-carboxylate instead of (S)-tert-butyl (1-(2-fluorophenyl)-2-hydroxyethyl)carbamate.

Step B-D. 5-[3-[(3S,5S)-5-(difluoromethyl)pyrrolidin-3-yl]oxy-5-methyl-isoxazol-4-yl]-N-(2,6-dimethylpyrimidin-4-yl)pyrazolo[1,5-a]pyridin-2-amine. The title compound was prepared (53.9 mg, 43%) as describe in Example 30 step B, D, and C (in order). MS (ESI): mass calcd. for C₂₂H₂₃F₂N₇O₂, 455.2. m/z found, 456.2 [M+H]⁺. ¹H NMR (400 MHz, DMSO-d6) δ 10.17 (s, 1H), 8.58 (dt, J=7.2, 0.9 Hz, 1H), 7.63 (dd, J=2.0, 0.9 Hz, 1H), 7.00 (s, 1H), 6.86 (dd, J=7.2, 2.0 Hz, 1H), 6.83 (s, 1H), 5.94-5.59 (m, 1H), 5.17 (s, 1H), 3.47-3.39 (m, 1H), 3.23 (dt, J=12.0, 6.4 Hz, 1H), 3.02 (d, J=12.0 Hz, 1H), 2.94 (s, 1H), 2.53 (s, 3H), 2.45 (s, 3H), 2.44-2.35 (m, 1H), 2.30 (s, 3H), 1.93 (dd, J=14.5, 4.7 Hz, 1H).

### Example 328: N-[5-[3-[(2S)-2-amino-2-cyclopropyl-ethoxy]-5-methyl-isoxazol-4-yl]pyrazolo[1,5-a]pyridin-2-yl]cyclopropanecarboxamide

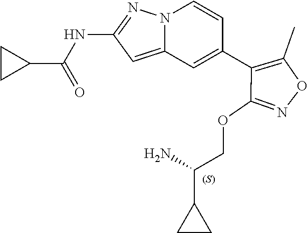

The title compound (53.9 mg, 3 steps, 38%) was prepared as described in Example 327 where tert-butyl (S)-(1-cyclopropyl-2-hydroxyethyl)carbamate was used instead of tert-butyl (2S,4R)-2-(difluoromethyl)-4-hydroxypyrrolidine-1-carboxylate. MS (ESI): mass calcd. for C₂₀H₂₃N₅O₃, 381.2. m/z found, 382.2 [M+H]⁺. ¹H NMR (500 MHz, DMSO-d6) δ 11.04 (s, 1H), 8.56 (dt, J=7.3, 1.0 Hz, 1H), 7.68 (dd, J=2.0, 0.9 Hz, 1H), 6.94 (dd, J=7.2, 2.0 Hz, 1H), 6.85 (s, 1H), 4.21 (dd, J=9.8, 5.3 Hz, 1H), 4.15 (dd, J=9.8, 6.5 Hz, 1H), 2.52 (s, 3H), 2.49-2.45 (m, 1H), 1.96-1.87 (m, 1H), 1.59 (s, 2H), 0.86-0.74 (m, 5H), 0.44-0.32 (m, 2H), 0.30-0.21 (m, 2H).

### Example 329: N-[6-[5-methyl-3-[[(2R)-1-methylazetidin-2-yl]methoxy]isoxazol-4-yl]imidazo[1,2-a]pyridin-2-yl]cyclopropanecarboxamide

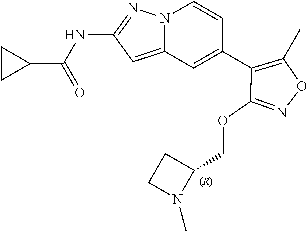

The title compound (51.9 mg, 4 steps, 18%) was prepared as described in Example 324, where (R)-tert-butyl 2-(hydroxymethyl)azetidine-1-carboxylate was used instead of (S)-tert-butyl (1-(2-fluorophenyl)-2-hydroxyethyl)carbamate. MS (ESI): mass calcd. for C₂₀H₂₃N₅O₃, 381.2. m/z found, 382.1 [M+H]⁺. ¹H NMR (400 MHz, DMSO-d6) δ 10.99 (s, 1H), 8.63 (s, 1H), 8.07 (s, 1H), 7.50 (d, J=9.2 Hz, 1H), 7.29 (d, J=9.2 Hz, 1H), 4.28-4.18 (m, 2H), 3.37-3.35 (m, 1H), 3.29-3.24 (m, 1H), 2.80-2.71 (m, 1H), 2.47 (s, 3H), 2.22 (s, 3H), 2.02-1.90 (m, 3H), 0.86-0.77 (m, 4H). 1H NMR (400 MHz, CDCl₃) δ 8.80 (br. s., 1H), 8.20 (s, 1H), 8.01 (s, 1H), 7.46 (d, J=9.2 Hz, 1H), 7.23 (dd, J=1.6, 9.2 Hz, 1H), 4.40-4.28 (m, 2H), 3.48-3.35 (m, 2H), 2.90-2.78 (m, 1H), 2.48 (s, 3H), 2.34 (s, 3H), 2.09-2.02 (m, 2H), 1.60-1.58 (m, 1H), 1.16-1.09 (m, 2H), 0.94-0.87 (m, 2H)

### Example 330: N-[5-[5-methyl-3-[(3S)-pyrrolidin-3-yl]oxy-isothiazol-4-yl]pyrazolo[1,5-a]pyridin-2-yl]cyclopropanecarboxamide

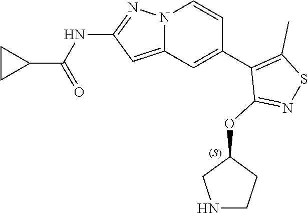

The title compound (263 mg, 3 steps, 52%) was prepared as described in Example 31 where (r)-tert-butyl 3-(tosyloxy)pyrrolidine-1-carboxylate was used instead of (S)-2-((tert-butoxycarbonyl)amino)-2-phenylethyl 4-methylbenzenesulfonate and 4-bromo-5-methylisothiazol-3-ol was used instead of 4-bromo-5-methylisoxazol-3-ol. MS (ESI): mass calcd. for C₁₉H₂₁N₅O₂S, 383.1. m/z found, 384.2 [M+H]⁺. ¹H NMR (500 MHz, DMSO-d6) δ 11.05 (s, 1H), 8.53 (d, J=7.1 Hz, 1H), 7.59 (d, J=1.9 Hz, 1H), 6.90-6.74 (m, 2H), 5.37-5.28 (m, 1H), 3.05 (dd, J=12.5, 5.3 Hz, 1H), 2.91-2.80 (m, 2H), 2.80-2.71 (m, 1H), 2.51 (s, 3H), 2.06-1.95 (m, 1H), 1.95-1.87 (m, 1H), 1.85-1.71 (m, 1H), 0.88-0.76 (m, 4H).

### Example 331: N-[5-[5-methyl-3-[[(2R)-morpholin-2-yl]methoxy]isothiazol-4-yl]pyrazolo[1,5-a]pyridin-2-yl]cyclopropanecarboxamide

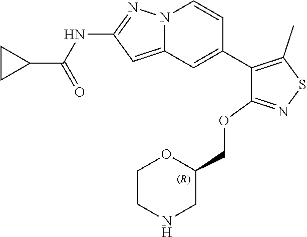

The title compound (265 mg, 4 steps, 28.6%) was prepared as described in Example 31 where tert-butyl (R)-2-((tosyloxy)methyl)morpholine-4-carboxylate was used instead of (S)-2-((tert-butoxycarbonyl)amino)-2-phenylethyl 4-methylbenzenesulfonate and 4-BROMO-5-METHYLISOTHIAZOL-3-OL was used instead of 4-bromo-5-methylisoxazol-3-ol. MS (ESI): mass calcd. for C₂₀H₂₃N₅O₃S, 413.2. m/z found, 414.2 [M+H]⁺. ¹H NMR (500 MHz, DMSO-d6) δ 11.06 (s, 1H), 8.57 (d, J=7.2 Hz, 1H), 7.60 (d, J=1.9 Hz, 1H), 6.89-6.80 (m, 2H), 4.34-4.25 (m, 2H), 3.75-3.64 (m, 2H), 3.47-3.38 (m, 1H), 2.81 (dd, J=12.3, 2.4 Hz, 1H), 2.68-2.55 (m, 2H), 2.52 (s, 3H), 2.49-2.45 (m, 1H), 1.96-1.88 (m, 1H), 0.87-0.78 (m, 4H).

### Example 332: N-[5-[5-methyl-3-[[(2R)-4-methylmorpholin-2-yl]methoxy]isothiazol-4-yl]pyrazolo[1,5-a]pyridin-2-yl]cyclopropanecarboxamide

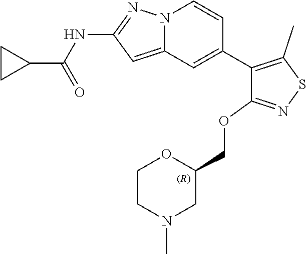

The title compound (42 mg, 4 steps, 82%) was prepared as described in Example 29 where (R)—N-(5-(5-methyl-3-(morpholin-2-ylmethoxy)isothiazol-4-yl)pyrazolo[1,5-a]pyridin-2-yl)cyclopropanecarboxamide Example 331 was used instead of N-[5-[3-[(3-methoxyazetidin-3-yl)methoxy]-5-methyl-isoxazol-4-yl]pyrazolo[1,5-a]pyridin-2-yl]cyclopropanecarboxamide. MS (ESI): mass calcd. for C₂₁H₂₅N₅O₃S, 427.2. m/z found, 428.2 [M+H]⁺. ¹H NMR (500 MHz, DMSO-d6) δ 11.06 (s, 1H), 8.57 (d, J=7.1 Hz, 1H), 7.61 (dd, J=2.0, 0.9 Hz, 1H), 6.86 (dd, J=7.2, 1.8 Hz, 2H), 4.34 (d, J=5.0 Hz, 2H), 3.81-3.73 (m, 2H), 3.54-3.45 (m, 1H), 2.74-2.68 (m, 1H), 2.56 (dd, J=11.3, 2.0 Hz, 1H), 2.52 (s, 3H), 2.16 (s, 3H), 1.98-1.88 (m, 2H), 1.88-1.81 (m, 1H), 0.86-0.77 (m, 4H).

### Example 333: N-[5-[2-methyl-4-[[(2R)-4-methylmorpholin-2-yl]methoxy]pyrazol-3-yl]pyrazolo[1,5-a]pyridin-2-yl]cyclopropanecarboxamide

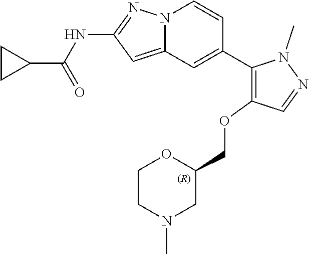

The title compound (11.5 mg, 4 steps, 5.3%) was prepared as described in Example 9 where (R)-tert-butyl 2-(hydroxymethyl)morpholine-4-carboxylate was used instead of tert-butyl (S)-2-(hydroxymethyl)azetidine-1-carboxylate. MS (ESI): mass calcd. for C₂₁H₂₆N₆O₃, 410.2. m/z found, 411.2 [M+H]⁺. ¹H NMR (400 MHz, DMSO-d6) δ 11.10 (s, 1H), 8.61 (d, J=7.6 Hz, 1H), 7.70 (d, J=1.2 Hz, 1H), 7.45 (s, 1H), 6.96 (dd, J=2.0, 7.6 Hz, 1H), 6.89 (s, 1H), 3.98-3.88 (m, 2H), 3.84 (s, 3H), 3.78-3.72 (m, 1H), 3.72-3.65 (m, 1H), 3.50-3.41 (m, 1H), 2.71-2.65 (m, 1H), 2.58-2.53 (m, 1H), 2.13 (s, 3H), 1.97-1.89 (m, 2H), 1.82-1.74 (m, 1H), 0.87-0.78 (m, 4H)

### Example 334: N-[5-[4-[(3-aminocyclobutyl)methoxy]-2-methyl-pyrazol-3-yl]pyrazolo[1,5-a]pyridin-2-yl]cyclopropanecarboxamide

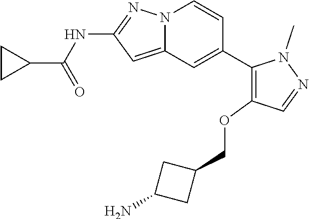 [SMILES: Cn1ncc(OC[C@H]2C[C@H](N)C2)c1-c1ccn2nc(NC(=O)C3CC3)cc2c1]

The title compound (23.2 mg, 3 steps, 12%0) was prepared as described in Example 6 where tert-butyl ((1r,3r)-3-(hydroxymethyl)cyclobutyl)carbamate was used instead of (R)-tert-butyl 2-(hydroxymethyl)morpholine-4-carboxylate. MS (ESI): mass calcd. for C₂₀H₂₄N₆O₂, 380.2. m/z found, 381.2 [M+H]⁺. ¹H NMR (400 MHz, DMSO-d6) δ 11.10 (s, 1H), 8.60 (d, J=7.6 Hz, 1H), 7.68 (s, 1H), 7.45 (s, 1H), 6.94 (dd, J=1.6, 7.2 Hz, 1H), 6.89 (s, 1H), 3.93 (d, J=6.8 Hz, 2H), 3.85 (s, 3H), 3.48-3.44 (m, 1H), 2.48-2.39 (m, 1H), 2.05-1.97 (m, 2H), 1.96-1.88 (m, 1H), 1.84-1.73 (m, 2H), 0.87-0.78 (m, 4H) 1H NMR (400 MHz, CD3OD) δ 8.45 (d, J=7.2 Hz, 1H), 7.65-7.57 (s, 1H), 7.42 (s, 1H), 6.95 (dd, J=2.0, 7.2 Hz, 1H), 6.90 (s, 1H), 3.98 (d, J=6.4 Hz, 2H), 3.88 (s, 3H), 3.59-3.48 (m, 1H), 2.66-2.51 (m, 1H), 2.22-2.12 (m, 2H), 2.01-1.92 (m, 2H), 1.91-1.83 (m, 1H), 1.04-0.96 (m, 2H), 0.94-0.86 (m, 2H)

### Example 335: N-[5-[2-methyl-4-[(3R)-pyrrolidin-3-yl]oxy-pyrazol-3-yl]pyrazolo[1,5-a]pyridin-2-yl]cyclopropanecarboxamide

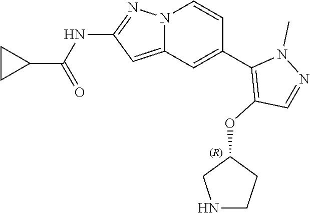

The title compound (23.2 mg, 3 steps, 40%) was prepared as described in Example 6 where (S)-tert-butyl 3-hydroxypyrrolidine-1-carboxylate was used instead of (R)-tert-butyl 2-(hydroxymethyl)morpholine-4-carboxylate. MS (ESI): mass calcd. for C₁₉H₂₂N₆O₂, 366.2. m/z found, 367.2 [M+H]⁺. ¹H NMR (400 MHz, DMSO-d6) δ 11.09 (s, 1H), 8.58 (d, J=7.2 Hz, 1H), 7.72-7.61 (m, 1H), 7.52-7.37 (m, 1H), 6.99-6.85 (m, 2H), 4.76-4.57 (m, 1H), 3.85 (s, 3H), 3.32-3.26 (m, 1H), 2.98-2.76 (m, 2H), 2.76-2.65 (m, 1H), 2.10-1.71 (m, 3H), 0.92-0.71 (m, 4H) 1H NMR (400 MHz, CD3OD) δ 8.45 (d, J=7.2 Hz, 1H), 7.62 (d, J=1.2 Hz, 1H), 7.42 (s, 1H), 6.94 (dd, J=2.0, 7.2 Hz, 1H), 6.92 (s, 1H), 4.78-4.67 (m, 1H), 3.87 (s, 3H), 3.19-3.10 (m, 1H), 3.10-2.99 (m, 2H), 2.99-2.88 (m, 1H), 2.09-1.94 (m, 2H), 1.92-1.81 (m, 1H), 1.03-0.97 (m, 2H), 0.93-0.85 (m, 2H) 1H NMR (400 MHz, DMSO-d6) δ 10.73 (s, 1H), 8.50 (d, J=7.2 Hz, 1H), 7.62 (d, J=0.4 Hz, 1H), 7.36 (s, 1H), 6.90 (dd, J=2.0, 7.2 Hz, 1H), 6.86 (s, 1H), 4.60 (s, 1H), 3.82 (s, 3H), 3.00-2.93 (m, 1H), 2.87 (s, 2H), 2.74 (s, 1H), 1.98-1.73 (m, 3H), 0.91-0.83 (m, 2H), 0.83-0.75 (m, 2H)

### Example 336: N-[5-[4-[(3S,5R)-1,5-dimethylpyrrolidin-3-yl]oxy-2-methyl-pyrazol-3-yl]pyrazolo[1,5-a]pyridin-2-yl]cyclopropanecarboxamide

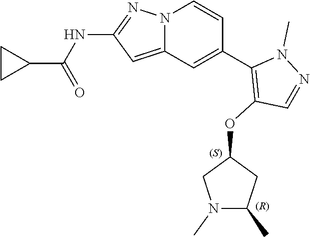

The title compound (55.8 mg, 6 steps, 33%) was prepared as described in Example 1 where (2R,4R)—N-boc-4-hydroxy-2-methylpyrrolidine was used instead of (2s,4r)-tert-butyl 4-hydroxy-2-methylpyrrolidine-1-carboxylate. MS (ESI): mass calcd. for C₂₁H₂₆N₆O₂, 394.2. m/z found, 395.2 [M+H]⁺. ¹H NMR (500 MHz, Methanol-d4) δ 8.45-8.41 (m, 1H), 7.67-7.64 (m, 1H), 7.33 (s, 1H), 6.98 (dd, J=7.2, 1.9 Hz, 1H), 6.91 (s, 1H), 4.64-4.57 (m, 1H), 3.87 (s, 3H), 3.25 (d, J=11.4 Hz, 1H), 2.59-2.41 (m, 2H), 2.28-2.22 (m, 4H), 1.91-1.83 (m, 1H), 1.64-1.54 (m, 1H), 1.14 (d, J=6.1 Hz, 3H), 1.02-0.98 (m, 2H), 0.94-0.86 (m, 2H).

### Example 337: (2*S, 3*R)—N-[5-[4-[(1,2-dimethylazetidin-3-yl)methoxy]-2-methyl-pyrazol-3-yl]pyrazolo[1,5-a]pyridin-2-yl]cyclopropanecarboxamide

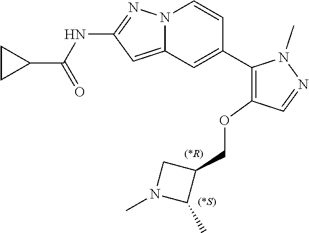

The title compound (30 mg, 4 steps, 15%) was prepared as described in Example 9 where Racemictert-butyl 3-(hydroxymethyl)-2-methylazetidine-1-carboxylate was used instead of tert-butyl (S)-2-(hydroxymethyl)azetidine-1-carboxylate. The diastereomers were separated at step B using SFC over AD 250 mm×30 mm, 5 μm (eluent: 35% to 35% (v/v) supercritical CO₂ in MEOH and H₂O with 0.1% NH₃) to afford pure P1 and P2, 70 mg and 80 mg. The racemate was further purified at step D using SFC over AD 250 mm×30 mm, 5 μm (eluent: 35% to 35% (v/v) supercritical CO₂ in MeOH and H₂O with 0.1% NH₃) to afford pure a white solid. MS (ESI): mass calcd. for C₂₁H₂₆N₆O₂, 394.2. m/z found, 395.3 [M+H]⁺. ¹H NMR (400 MHz, CD3OD) δ 8.46 (d, J=7.2 Hz, 1H), 7.64 (d, J=0.8 Hz, 1H), 7.41 (s, 1H), 6.96 (dd, J=1.6, 7.2 Hz, 1H), 6.91 (s, 1H), 4.02-3.93 (m, 2H), 3.89 (s, 3H), 3.46-3.40 (m, 1H), 3.07-2.99 (m, 1H), 2.77-2.72 (m, 1H), 2.47-2.38 (m, 1H), 2.25 (s, 3H), 1.90-1.83 (m, 1H), 1.15 (d, J=6.4 Hz, 3H), 1.02-0.97 (m, 2H), 0.93-0.87 (m, 2H)

### Example 338: N-[5-[2-methyl-4-[(3S,5S)-5-methylpyrrolidin-3-yl]oxy-pyrazol-3-yl]pyrazolo[1,5-a]pyridin-2-yl]cyclopropanecarboxamide

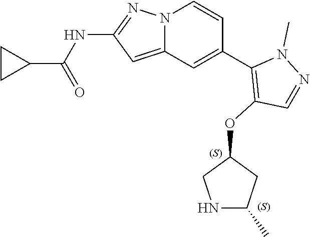

The title compound (281 mg, 5 steps, 28%) was prepared as described in Example 1, where reductive amination step, Step F was not performed. MS (ESI): mass calcd. for C₂₀H₂₄N₆O₂, 380.2. m/z found, 381.2 [M+H]⁺. ¹H NMR (500 MHz, Methanol-d4) δ 8.48-8.42 (m, 1H), 7.64-7.58 (m, 1H), 7.38 (s, 1H), 6.93 (dd, J=7.2, 1.9 Hz, 1H), 6.91 (s, 1H), 4.73 (t, J=5.6 Hz, 1H), 3.87 (s, 3H), 3.30-3.21 (m, 2H), 2.96-2.92 (m, 1H), 2.13 (dd, J=13.7, 5.8 Hz, 1H), 1.91-1.83 (m, 1H), 1.51-1.42 (m, 1H), 1.14 (d, J=6.4 Hz, 3H), 1.02-0.98 (m, 2H), 0.93-0.87 (m, 2H).

### Example 339: 5-[2-methyl-4-[(3S)-1-methylpyrrolidin-3-yl]oxy-pyrazol-3-yl]-N-[5-(trifluoromethyl)-2-pyridyl]pyrazolo[1,5-a]pyridin-2-amine

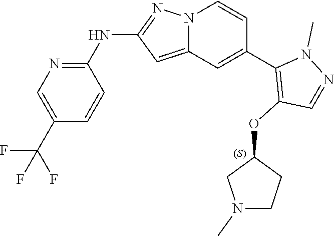

The title compound (100 mg, 53%) was prepared as described in Example 7 where 2-bromo-5-(trifluoromethyl)pyridine was used instead of 2-chloro-6-(trifluoromethyl)pyrazine. MS (ESI): mass calcd. for C₂₂H₂₂F₃N₇O, 457.2. m/z found, 458.2 [M+H]⁺. ¹H NMR (400 MHz, DMSO-d6) δ 10.46 (s, 1H), 8.62 (d, J=7.2 Hz, 1H), 8.58-8.56 (m, 1H), 7.97 (dd, J=2.4, 8.8 Hz, 1H), 7.69 (d, J=0.8 Hz, 1H), 7.44 (d, J=8.8 Hz, 1H), 7.40 (s, 1H), 6.95 (s, 1H), 6.93 (dd, J=2.0, 6.8 Hz, 1H), 4.70-4.64 (m, 1H), 3.86 (s, 3H), 2.70-2.64 (m, 1H), 2.63-2.57 (m, 2H), 2.34-2.26 (m, 1H), 2.21 (s, 3H), 2.20-2.13 (m, 1H), 1.82-1.72 (m, 1H) 1H NMR (376 MHz, DMSO-d6) δ−59.58 (s, 3F)

### Example 340: 5-[2-methyl-4-[(3S)-1-methylpyrrolidin-3-yl]oxy-pyrazol-3-yl]-N-[4-(trifluoromethyl)-2-pyridyl]pyrazolo[1,5-a]pyridin-2-amine

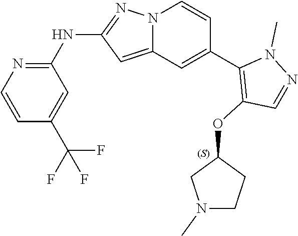

The title compound (73 mg, 33%) was prepared as described in Example 7 where 2-bromo-4-(trifluoromethyl)pyridine was used instead of 2-chloro-6-(trifluoromethyl)pyrazine. MS (ESI): mass calcd. for C₂₂H₂₂F₃N₇O, 457.2. m/z found, 458.2 [M+H]⁺. ¹H NMR (400 MHz, DMSO-d6) δ 10.32 (s, 1H), 8.62 (d, J=7.2 Hz, 1H), 8.47 (d, J=5.2 Hz, 1H), 7.67 (d, J=0.8 Hz, 1H), 7.64 (s, 1H), 7.39 (s, 1H), 7.12-7.08 (m, 1H), 6.94-6.89 (m, 2H), 4.73-4.51 (m, 1H), 3.86 (s, 3H), 2.70-2.64 (m, 1H), 2.63-2.57 (m, 2H), 2.35-2.25 (m, 1H), 2.22 (s, 3H), 2.19-2.12 (m, 1H), 1.84-1.71 (m, 1H) 19F NMR (376 MHz, DMSO-d6) δ −63.8 (s, 3F)

### Example 341: N-[5-[2-methyl-4-[[(2S,3S)-3-methylazetidin-2-yl]methoxy]pyrazol-3-yl]pyrazolo[1,5-a]pyridin-2-yl]cyclopropanecarboxamide

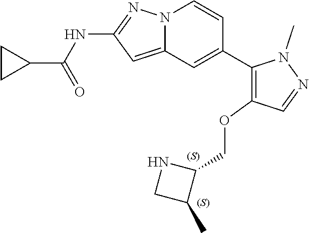

The title compound (73 mg, 33%) was prepared as described in Example 3 Step A and B, where tert-butyl (2S,3S)-2-(hydroxymethyl)-3-methylazetidine-1-carboxylate was used instead of tert-butyl (S)-2-(hydroxymethyl)-3,3-dimethylazetidine-1-carboxylate. MS (ESI): mass calcd. for C₂₀H₂₄N₆O₂, 380.2. m/z found, 381.2 [M+H]⁺. ¹H NMR (400 MHz, Methanol-d4) δ 8.44 (d, J=7.2 Hz, 1H), 7.68-7.59 (m, 1H), 7.44 (s, 1H), 6.97 (dd, J=7.2, 1.9 Hz, 1H), 6.90 (s, 1H), 4.02 (d, J=5.3 Hz, 2H), 3.88 (s, 3H), 3.73 (d, J=17.2 Hz, 1H), 3.20 (s, 1H), 2.68-2.47 (m, 1H), 1.86 (dq, J=8.0, 4.6, 4.1 Hz, 1H), 1.14 (d, J=6.7 Hz, 3H), 0.99 (dt, J=4.6, 3.1 Hz, 2H), 0.90 (dt, J=8.0, 3.2 Hz, 2H).

### Example 342: N-[5-[2-methyl-4-[(3S)-1-methylpyrrolidin-3-yl]oxy-pyrazol-3-yl]pyrazolo[1,5-a]pyridin-2-yl]cinnolin-3-amine

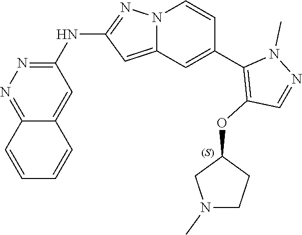

The title compound (56 mg, 32%) was prepared as described in Example 7 where 3-chlorocinnoline was used instead of 2-chloro-6-(trifluoromethyl)pyrazine. MS (ESI): mass calcd. for C₂₄H₂₄N₈O, 440.2. m/z found, 441.2 [M+H]⁺. ¹H NMR (400 MHz, DMSO-d6) δ 10.59 (s, 1H), 8.68 (d, J=7.2 Hz, 1H), 8.38 (s, 1H), 8.29 (d, J=8.4 Hz, 1H), 7.91 (d, J=8.4 Hz, 1H), 7.75-7.61 (m, 3H), 7.40 (s, 1H), 6.92 (dd, J=1.6, 7.2 Hz, 1H), 6.58 (s, 1H), 4.71-4.64 (m, 1H), 3.87 (s, 3H), 2.70-2.65 (m, 1H), 2.64-2.58 (m, 2H), 2.35-2.26 (m, 1H), 2.25-2.13 (m, 4H), 1.84-1.73 (m, 1H)

### Example 343: N-[5-[2-methyl-4-[(3S)-1-methylpyrrolidin-3-yl]oxy-pyrazol-3-yl]pyrazolo[1,5-a]pyridin-2-yl]quinoxalin-2-amine

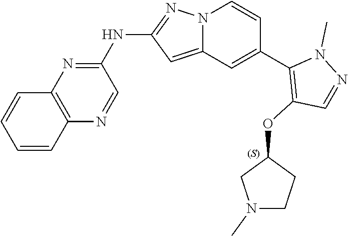

The title compound (61 mg, 36%) was prepared as described in Example 7 where 2-chloroquinoxaline was used instead of 2-chloro-6-(trifluoromethyl)pyrazine. MS (ESI): mass calcd. for C₂₄H₂₄N₈O, 440.2. m/z found, 441.3 [M+H]⁺. ¹H NMR (400 MHz, DMSO-d6) δ 10.85 (s, 1H), 8.77 (s, 1H), 8.66 (d, J=7.2 Hz, 1H), 7.91 (d, J=8.0 Hz, 1H), 7.83 (d, J=8.4 Hz, 1H), 7.77 (s, 1H), 7.73-7.67 (m, 1H), 7.56-7.49 (m, 1H), 7.46 (s, 1H), 7.42 (s, 1H), 6.98 (d, J=7.2 Hz, 1H), 4.75-4.65 (m, 1H), 3.88 (s, 3H), 2.78-2.69 (m, 2H), 2.45-2.32 (m, 2H), 2.28 (s, 3H), 2.26-2.16 (m, 1H), 1.87-1.77 (m, 1H) 1H NMR (400 MHz, CD3OD): 8.65 (s, 1H), 8.49 (d, J=6.8 Hz, 1H), 7.92-7.84 (m, 2H), 7.72-7.65 (m, 2H), 7.54-7.48 (m, 1H), 7.47 (s, 1H), 7.39 (s, 1H), 6.96 (dd, J=1.6, 7.2 Hz, 1H), 4.78-4.73 (m, 1H), 3.91 (s, 3H), 3.01-2.96 (m, 1H), 2.95-2.88 (m, 1H), 2.87-2.81 (m, 1H), 2.58-2.50 (m, 1H), 2.41 (s, 3H), 2.35-2.25 (m, 1H), 2.07-1.98 (m, 1H)

### Example 344: 5-[2-methyl-4-[(3S)-1-methylpyrrolidin-3-yl]oxy-pyrazol-3-yl]-N-pyrazolo[1,5-a]pyridin-2-yl-pyrazolo[1,5-a]pyridin-2-amine

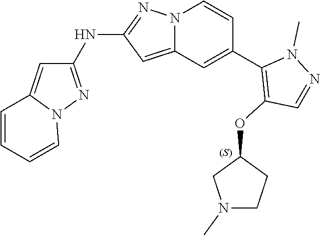

The title compound (75 mg, 45%) was prepared as described in Example 7 where 2-chloropyrazolo[1,5-a]pyridine was used instead of 2-chloro-6-(trifluoromethyl)pyrazine. MS (ESI): mass calcd. for C₂₃H₂₄N₈O, 428.2. m/z found, 429.2 [M+H]⁺. ¹H NMR (400 MHz, DMSO-d6) δ 9.65 (s, 1H), 8.56 (d, J=7.2 Hz, 1H), 8.48 (d, J=6.8 Hz, 1H), 7.59 (d, J=0.8 Hz, 1H), 7.47 (d, J=8.8 Hz, 1H), 7.39 (s, 1H), 7.19-7.08 (m, 1H), 6.83 (dd, J=2.0, 7.2 Hz, 1H), 6.73-6.64 (m, 1H), 6.58 (s, 1H), 6.45 (s, 1H), 4.72-4.62 (m, 1H), 3.85 (s, 3H), 2.71-2.64 (m, 1H), 2.63-2.57 (m, 2H), 2.34-2.26 (m, 1H), 2.21 (s, 3H), 2.20-2.12 (m, 1H), 1.82-1.72 (m, 1H)

### Example 345: N-[5-[4-[[(3R,4R)-4-hydroxypyrrolidin-3-yl]methoxy]-2-methyl-pyrazol-3-yl]pyrazolo[1,5-a]pyridin-2-yl]cyclopropanecarboxamide

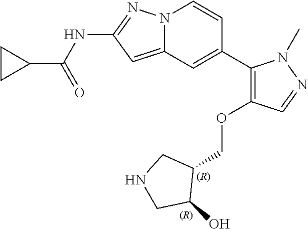

The title compound (335 mg, 3 steps, 35%) was prepared as described in Example 6 where TFA/DCM was used instead of HCl/1,4-dioxane and (3R,4R)-tert-butyl 3-hydroxy-4-(hydroxymethyl)pyrrolidine-1-carboxylate was used instead of (R)-tert-butyl 2-(hydroxymethyl)morpholine-4-carboxylate. MS (ESI): mass calcd. for C₂₀H₂₄N₆O₃, 396.2. m/z found, 397.2 [M+H]⁺. ¹H NMR (400 MHz, DMSO-d6) δ 11.10 (s, 1H), 8.59 (d, J=6.8 Hz, 1H), 7.69 (s, 1H), 7.45 (s, 1H), 6.95 (d, J=6.4 Hz, 1H), 6.90 (s, 1H), 5.39-4.50 (m, 1H), 3.96-3.80 (m, 6H), 3.17-2.96 (m, 2H), 2.88-2.78 (m, 1H), 2.58-2.55 (m, 1H), 2.41-2.13 (m, 1H), 1.98-1.88 (m, 1H), 0.86-0.80 (m, 4H) 1H NMR (400 MHz, CD3OD): 8.48-8.42 (m, 1H), 7.64-7.58 (m, 1H), 7.46-7.42 (m, 1H), 6.97-6.89 (m, 2H), 4.23-4.12 (m, 1H), 4.03-3.91 (m, 2H), 3.87 (s, 3H), 3.64-3.57; 3.28-3.16 (m, 1H), 3.11-3.01 (m, 1H), 2.94-2.76 (m, 2H), 2.47-2.39 (m, 1H), 1.91-1.83 (m, 1H), 1.02-0.96 (m, 2H), 0.94-0.86 (m, 2H)

### Example 346: (R)—N-(5-(4-(azetidin-2-ylmethoxy)-1-methyl-1H-pyrazol-5-yl)pyrazolo[1,5-a]pyridin-2-yl)cyclopropanecarboxamide

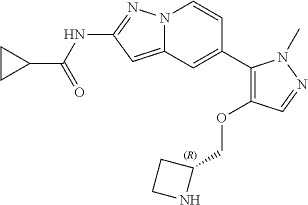

The title compound (460 mg, 5 steps, 76%) was prepared as described in Example 5 where tert-butyl (R)-2-(hydroxymethyl)azetidine-1-carboxylate was used instead of trans-rac-tert-butyl (3r,4r)-3-hydroxy-4-methoxypyrrolidine-1-carboxylate. MS (ESI): mass calcd. for C₁₉H₂₂N₆O₂, 366.2. m/z found, 367.20 [M+H]⁺. ¹H NMR (400 MHz, Chloroform-d) δ 9.24 (s, 1H), 8.28 (d, J=7.2 Hz, 1H), 7.48 (dd, J=2.0, 0.9 Hz, 1H), 7.35 (s, 1H), 6.98 (s, 1H), 6.83 (dd, J=7.2, 1.9 Hz, 1H), 4.24 (dd, J=6.9, 4.6 Hz, 1H), 4.02 (qd, J=9.9, 5.5 Hz, 2H), 3.90 (s, 3H), 3.66 (t, J=8.1 Hz, 1H), 3.56-3.37 (m, 1H), 2.95 (s, 2H), 2.46-2.28 (m, 1H), 2.28-2.14 (m, 1H), 1.61 (s, OH), 1.12 (dt, J=6.8, 3.5 Hz, 2H), 0.91-0.81 (m, 2H).

### Example 347: N-[5-[4-[[(2R)-1-cyclopropylazetidin-2-yl]methoxy]-2-methyl-pyrazol-3-yl]pyrazolo[1,5-a]pyridin-2-yl]cyclopropanecarboxamide

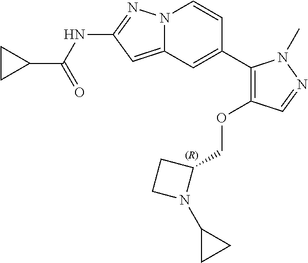

(1-ethoxycyclopropoxy)trimethylsilane (71 mg, 0.41 mmol) was added to a solution consisting of (R)—N-(5-(4-(azetidin-2-ylmethoxy)-1-methyl-1H-pyrazol-5-yl)pyrazolo[1,5-a]pyridin-2-yl)cyclopropanecarboxamide (Example 346, 100 mg, 0.208 mmol) and MeOH (5 mL). The reaction mixture was stirred at room-temperature for 30 min. Then NaBH₃CN (34 mg, 0.54 mmol) was added to the reaction mixture. The reaction mixture was stirred at 40° C. for 16 hours. The reaction mixture was purified by preparative HPLC using a Boston Prime C18 150×30 mm×5 μm column (eluent: 35% to 65% (v/v) CH₃CN and H₂O with 0.05% NH₃) to afford pure product. The product was suspended in water (10 mL), the mixture frozen using dry ice/ethanol, and then lyophilized to dryness to afford the title compound (18.5 mg, 22%) as a white solid. MS (ESI): mass calcd. for C₂₂H₂₆N₆O₂, 406.2. m/z found, 407.1 [M+H]⁺. ¹H NMR (400 MHz, DMSO-d6) δ 11.10 (s, 1H), 8.61 (d, J=7.2 Hz, 1H), 7.74-7.66 (m, 1H), 7.43 (s, 1H), 6.98 (dd, J=1.6, 7.2 Hz, 1H), 6.88 (s, 1H), 3.95-3.87 (m, 2H), 3.85 (s, 3H), 3.59-3.48 (m, 1H), 3.23-3.14 (m, 1H), 3.01-2.89 (m, 1H), 2.00-1.89 (m, 2H), 1.88-1.73 (m, 2H), 0.87-0.77 (m, 4H), 0.27-0.15 (m, 3H), 0.13-0.05 (m, 1H)

### Example 348: N-[5-[4-[(2S)-2-amino-3,3-dimethyl-butoxy]-2-methyl-pyrazol-3-yl]pyrazolo[1,5-a]pyridin-2-yl]cyclopropanecarboxamide

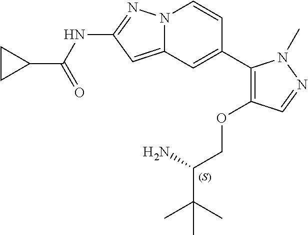

The title compound (73 mg, 33%) was prepared as described in Example 3 step A and B, where (S)-tert-butyl (1-hydroxy-3,3-dimethylbutan-2-yl)carbamate was used instead of tert-butyl (S)-2-(hydroxymethyl)-3,3-dimethylazetidine-1-carboxylate. MS (ESI): mass calcd. for C₂₁H₂₈N₆O₂, 396.2. m/z found, 397.1 [M+H]⁺. ¹H NMR (400 MHz, DMSO-d6) δ 11.11 (s, 1H), 8.63-8.47 (m, 1H), 7.80-7.67 (m, 1H), 7.48 (s, 1H), 7.06-6.95 (m, 1H), 6.90 (s, 1H), 4.10-4.02 (m, 1H), 3.91-3.83 (m, 3H), 3.71-3.63 (m, 1H), 2.72-2.63 (m, 1H), 1.99-1.88 (m, 1H), 0.93-0.79 (m, 13H) t=80° C. 1H NMR (400 MHz, DMSO-d6) δ 10.79-10.64 (m, 1H), 8.51 (d, J=6.8 Hz, 1H), 7.64 (s, 1H), 7.41 (s, 1H), 6.97-6.90 (m, 1H), 6.86 (s, 1H), 4.08-3.99 (m, 1H), 3.86-3.80 (m, 3H), 3.74-3.65 (m, 1H), 2.69-2.64 (m, 1H), 1.99-1.90 (m, 1H), 0.88-0.78 (m, 13H)

### Example 349: N-(5,6-dimethylpyrazin-2-yl)-5-[2-methyl-4-[(3S)-1-methylpyrrolidin-3-yl]oxy-pyrazol-3-yl]pyrazolo[1,5-a]pyridin-2-amine

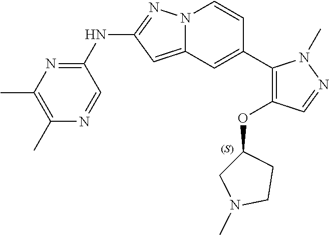

The title compound (36 mg, 26%) was prepared as described in Example 7 where 5-chloro-2,3-dimethylpyrazine was used instead of 2-chloro-6-(trifluoromethyl)pyrazine. MS (ESI): mass calcd. for C₂₂H₂₆N₈O, 418.2. m/z found, 419.1 [M+H]⁺. ¹H NMR (400 MHz, DMSO-d6) δ 9.96 (s, 1H), 8.59 (d, J=7.2 Hz, 1H), 8.43 (s, 1H), 7.64 (d, J=0.8 Hz, 1H), 7.39 (s, 1H), 6.88 (dd, J=2.0, 7.2 Hz, 1H), 6.85 (s, 1H), 4.71-4.63 (m, 1H), 3.85 (s, 3H), 2.69-2.59 (m, 3H), 2.43 (s, 3H), 2.38 (s, 3H), 2.33-2.25 (m, 1H), 2.21 (s, 3H), 2.20-2.13 (m, 1H), 1.82-1.72 (m, 1H)

### Example 350: N-[5-[4-[[(2R)-1-(2-fluoroethyl)pyrrolidin-2-yl]methoxy]-2-methyl-pyrazol-3-yl]pyrazolo[1,5-a]pyridin-2-yl]cyclopropanecarboxamide

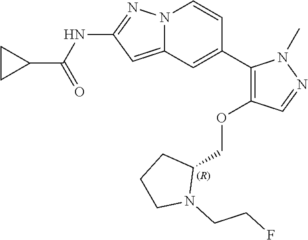

Step A and B. (R)—N-(5-(1-methyl-4-(pyrrolidin-2-ylmethoxy)-1H-pyrazol-5-yl)pyrazolo[1,5-a]pyridin-2-yl)cyclopropanecarboxamide. The title compound (40 mg, 31%) was prepared as described in Example 3 step A and B, where N-(5-(4-hydroxy-1-methyl-1H-pyrazol-5-yl)pyrazolo[1,5-a]pyridin-2-yl)cyclopropanecarboxamide was used instead of tert-butyl (S)-2-(hydroxymethyl)-3,3-dimethylazetidine-1-carboxylate. MS (ESI): mass calcd. for C₂₀H₂₄N₆O₂, 380.2. m/z found, 481.3 [M+H]⁺. 1H NMR (500 MHz, Methanol-d4) δ 8.44 (dd, J=7.2, 1.0 Hz, 1H), 7.64 (dd, J=1.9, 0.9 Hz, 1H), 7.43 (s, 1H), 6.96 (dd, J=7.2, 1.9 Hz, 1H), 6.90 (s, 1H), 3.96 (dd, J=9.5, 4.7 Hz, 1H), 3.93-3.88 (m, 1H), 3.42 (qd, J=7.3, 4.6 Hz, 1H), 2.97-2.80 (m, 2H), 1.99-1.68 (m, 4H), 1.53 (ddt, J=12.5, 8.7, 7.2 Hz, 1H), 0.99 (dt, J=4.5, 3.2 Hz, 2H), 0.90 (dt, J=8.0, 3.3 Hz, 2H).

Step C. N-[5-[4-[[(2R)-1-(2-fluoroethyl)pyrrolidin-2-yl]methoxy]-2-methyl-pyrazol-3-yl]pyrazolo[1,5-a]pyridin-2-yl]cyclopropanecarboxamide. A mixture consisting of (R)—N-(5-(1-methyl-4-(pyrrolidin-2-ylmethoxy)-1H-pyrazol-5-yl)pyrazolo[1,5-a]pyridin-2-yl)cyclopropanecarboxamide (150 mg, 0.394 mmol), 1-fluoro-2-iodoethane (82.3 mg, 0.473 mmol), DIEA (153 mg, 1.18 mmol) and DMF (3 mL) was stirred at r.t. overnight. The mixture was purified by preparative HPLC with Boston Prime C18 150×30 mm×5 um, column (eluent: 35% to 65% (v/v) CH₃CN and H₂O with 0.04% NH₃H₂O+10 mM NH₄HCO₃) to afford titled product. The product was suspended in water (10 mL), the mixture frozen using dry ice/EtOH, and then lyophilized to dryness to afford P1 (51.9 mg, 31%) as a white solid. MS (ESI): mass calcd. for C₂₂H₂₇FN₆O₂, 426.2. m/z found, 427.2 [M+H]⁺. ¹H NMR (400 MHz, DMSO-d6) δ 11.10 (s, 1H), 8.59 (d, J=7.6 Hz, 1H), 7.68 (d, J=0.8 Hz, 1H), 7.45 (s, 1H), 6.95 (dd, J=2.0, 7.2 Hz, 1H), 6.88 (s, 1H), 4.54-4.43 (m, 1H), 4.41-4.32 (m, 1H), 3.93-3.87 (m, 1H), 3.85 (s, 3H), 3.82-3.76 (m, 1H), 3.19-3.01 (m, 2H), 2.86-2.77 (m, 1H), 2.68-2.55 (m, 1H), 2.32-2.24 (m, 1H), 1.98-1.89 (m, 1H), 1.88-1.79 (m, 1H), 1.72-1.61 (m, 2H), 1.58-1.49 (m, 1H), 0.88-0.78 (m, 4H) 19F NMR (376 MHz, DMSO-d6) δ −217.96-−218.08 (m, 1F)

### Example 351: N-[5-[4-[[(3R,4R)-4-methoxypyrrolidin-3-yl]methoxy]-2-methyl-pyrazol-3-yl]pyrazolo[1,5-a]pyridin-2-yl]cyclopropanecarboxamide

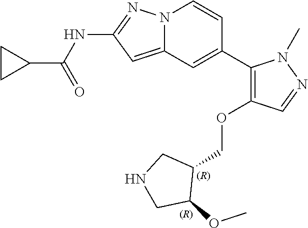

Step A. tert-butyl (3R,4R)-3-hydroxy-4-(((1-methyl-1H-pyrazol-4-yl)oxy)methyl)pyrrolidine-1-carboxylate. A mixture consisting of 1-methyl-1H-pyrazol-4-ol (400 mg, 4.08 mmol), (3R,4R)-tert-butyl 3-hydroxy-4-((tosyloxy)methyl)pyrrolidine-1-carboxylate (1.8 g, 4.9 mmol), Cs₂CO₃ (3.98 g, 12.2 mmol), and DMF (5 mL) was stirred overnight at 50° C. The reaction mixture was concentrated under reduced pressure to give the crude product, which was purified by FCC (eluent: petroleum ether:ethyl acetate=100:0 to 10:90) to afford the title compound (900 mg, 74%) as a white solid. MS (ESI): mass calcd. for C₁₄H₂₃N₃O₄, 297.2. m/z found, 242.1 [M−tBu+H]⁺. 1H NMR (400 MHz, DMSO-d6) δ 7.44 (s, 1H), 7.17 (s, 1H), 5.17 (d, J=3.2 Hz, 1H), 4.09-4.00 (m, 1H), 3.84-3.77 (m, 1H), 3.72 (s, 3H), 3.51-3.42 (m, 2H), 3.14-3.05 (m, 2H), 2.89 (s, 1H), 2.41-2.31 (m, 1H), 1.39 (s, 9H).

Step B. tert-butyl (3R,4R)-3-methoxy-4-(((1-methyl-1H-pyrazol-4-yl)oxy)methyl)pyrrolidine-1-carboxylate. NaH (121 mg, 60% in mineral oil, 3.03 mmol) was added in portions to a stirred mixture consisting of (3R,4R) tert butyl 3-hydroxy-4-(((1-methyl-1H-pyrazol-4-yl)oxy)methyl)pyrrolidine-1-carboxylate from Step A (300 mg, 1.01 mmol), and THE (5 mL) at 0° C. and stirred room-temperature for 30 min. Then iodomethane (4.14 g, 29.2 mmol) was add to the reaction mixture and stirred overnight at room-temperature. The mixture was added aq. NH₄Cl (10 mL) at 0° C. and stirred for 0.5 h. The reaction mixture was concentrated to dryness under reduced pressure to give the crude product, which was purified by FCC (petroleum ether:ethyl acetate-=1:0 to 1:1) to afford product (250 mg, 79%) as a yellow oil. MS (ESI): mass calcd. for C₁₅H₂₅N₃O₄, 311.2. m/z found, 256.2 [M−tBu+H]⁺.

Step C. tert-butyl (3R,4R)-3-(((5-bromo-1-methyl-1H-pyrazol-4-yl)oxy)methyl)-4-methoxypyrrolidine-1-carboxylate. A solution consisting of (3R,4R)-tert-butyl 3-methoxy-4-(((1-methyl-1H-pyrazol-4-yl)oxy)methyl)pyrrolidine-1-carboxylate from step B (220 mg, 0.707 mmol), NBS (128 mg, 0.721 mmol), and ACN (1 mL) was stirred at room temperature for 2 h. The reaction mixture was concentrated under reduced pressure to give the crude product. The product was added H₂O (10 mL) and extracted with EtOAc (10 mL×2). The combined organic phases were dried over Na₂SO₄, filtered, and concentrated to afford a yellow oil (240 mg, crude). MS (ESI): mass calcd. for C₁₅H₂₄BrN₃O₄, 389.1. m/z found, 336.1 [M−tBu+H]⁺.

Step D. tert-butyl (3R,4R)-3-(((5-(2-(cyclopropanecarboxamido)pyrazolo[1,5-a]pyridin-5-yl)-1-methyl-1H-pyrazol-4-yl)oxy)methyl)-4-methoxypyrrolidine-1-carboxylate. A solution consisting of N-(5-(4,4,5,5-tetramethyl-1,3,2-dioxaborolan-2-yl)pyrazolo[1,5-a]pyridin-2-yl)cyclopropanecarboxamide Intermediate 3 (176 mg, 0.538 mmol), (3R,4R)-tert-butyl 3-(((5-bromo-1-methyl-1H-pyrazol-4-yl)oxy)methyl)-4-methoxypyrrolidine-1-carboxylate (210 mg, 0.538 mmol), Pd(dppf)Cl₂ (39 mg, 0.054 mmol), K₃PO₄ (342 mg, 1.61 mmol) and 1,4-dioxane/H₂O (5 mL) was stirred vigorously under a stream of nitrogen for 10 mm. The vial was placed in a pre-heated oil bath at 80° C. and heated at that temperature overnight. The reaction mixture was concentrated to dryness under reduced pressure to give the crude product. The crude was purified by FCC (petroleum ether:ethyl acetate=1:0 to 1:1) to afford product (240 mg, 87%) as a yellow oil. MS (ESI): mass calcd. for C₂₆H₃₄N₆O₅, 510.3. m/z found, 455.2 [M−tBu+H]⁺.

Step E. N-(5-(4-(((3R,4R)-4-methoxypyrrolidin-3-yl)methoxy)-1-methyl-1H-pyrazol-5-yl)pyrazolo[1,5-a]pyridin-2-yl)cyclopropanecarboxamide. (3R,4R)-tert-butyl 3-(((5-(2-(cyclopropanecarboxamido)pyrazolo[1,5-a]pyridin-5-yl)-1-methyl-1H-pyrazol-4-yl)oxy)methyl)-4-methoxypyrrolidine-1-carboxylate from Step D (240 mg, 0.470 mmol) was added to a solution of TFA/DCM (8 mL, TFA:DCM=1:3), The resulting mixture was stirred at room-temperature for 2 hours. The reaction mixture was purified by preparative HPLC using a Phenomenex Gemini-NX C18 75×30 mm×3 um column (eluent: 20% to 50% (v/v) CH₃CN and H₂O with 0.04% NH₃ and 10 mM NH₄HCO₃) to afford pure product. The product was suspended in water (10 mL), the mixture frozen using dry ice/ethanol, and then lyophilized to dryness to afford the title compound (48.6 mg, 24%) as a white solid. MS (ESI): mass calcd. for C₂₁H₂₆N₆O₃, 410.2. m/z found, 411.1 [M+H]⁺. ¹H NMR (400 MHz, CD3OD): 8.47 (d, J=6.8 Hz, 1H), 7.64 (d, J=1.2 Hz, 1H), 7.46 (s, 1H), 6.96-6.90 (m, 2H), 4.02-3.94 (m, 2H), 3.90-3.86 (m, 4H), 3.35-3.32 (m, 1H), 3.26 (s, 3H), 3.16-3.10 (m, 1H), 3.08-3.02 (m, 1H), 2.89-2.82 (m, 1H), 2.65-2.56 (m, 1H), 1.91-1.84 (m, 1H), 1.03-0.96 (m, 2H), 0.95-0.87 (m, 2H) 1H NMR (400 MHz, CDCl₃) δ 8.81 (s, 1H), 8.40 (d, J=6.8 Hz, 1H), 7.45 (s, 1H), 7.35 (s, 1H), 6.99 (s, 1H), 6.83 (d, J=6.8 Hz, 1H), 3.94 (d, J=6.0 Hz, 2H), 3.89 (s, 3H), 3.83-3.78 (m, 1H), 3.42-3.39 (m, 1H), 3.25 (s, 3H), 3.19-3.07 (m, 2H), 2.96-2.89 (m, 1H), 2.62-2.53 (m, 1H), 1.71-1.60 (m, 1H), 1.15-1.08 (m, 2H), 0.93-0.86 (m, 2H)

### Example 352: (1*S, 2*S, 4*R)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine

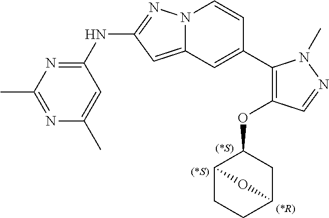

The title compound (23.7 mg, 5%) was prepared as described in Example 6 step B where (rel-exo)-7-oxabicyclo[2.2.1]heptan-2-yl 4-methylbenzenesulfonate was used instead of (R)-tert-butyl 2-((tosyloxy)methyl)morpholine-4-carboxylate. The enantiomers were separated by SFC over DAICEL CHIRALPAK IG 250 mm×30 mm×10 μm (eluent: 55% to 55% (v/v) supercritical CO₂ in EtOH and H₂O with 0.1% NH₃). The pure fractions were collected and the volatiles were removed under reduced pressure. The product was suspended in water (10 mL), the mixture frozen using dry ice/ethanol, and then lyophilized to dryness to afford the title compound as a white solid. MS (ESI): mass calcd. for C₂₃H₂₅N₇O₂, 431.2. m/z found, 432.1 [M+H]⁺. ¹H NMR (400 MHz, DMSO-d6) δ 10.30 (s, 1H), 8.70 (d, J=7.2 Hz, 1H), 7.75 (d, J=0.8 Hz, 1H), 7.52 (s, 1H), 7.06 (s, 1H), 7.03-6.94 (m, 2H), 4.78-4.68 (m, 1H), 4.63-4.52 (m, 2H), 3.92 (s, 3H), 2.53 (s, 3H), 2.37 (s, 3H), 2.27-2.15 (m, 1H), 2.12-2.04 (m, 1H), 1.73-1.63 (m, 1H), 1.61-1.49 (m, 2H), 1.36-1.31 (m, 1H) 1H NMR (400 MHz, CD3OD): 8.49 (d, J=7.2 Hz, 1H), 7.61 (d, J=1.2 Hz, 1H), 7.39 (s, 1H), 7.03 (s, 1H), 6.98-6.85 (m, 2H), 4.67-4.64 (m, 1H), 4.58-4.50 (m, 2H), 3.89 (s, 3H), 2.53 (s, 3H), 2.38 (s, 3H), 2.26-2.12 (m, 2H), 1.79-1.65 (m, 1H), 1.64-1.49 (m, 2H), 1.42-1.34 (m, 1H)

### Example 353: (*S)—N-[5-[2-methyl-4-[[1-(tetrahydrofuran-2-ylmethyl)azetidin-2-yl]methoxy]pyrazol-3-yl]pyrazolo[1,5-a]pyridin-2-yl]cyclopropanecarboxamide

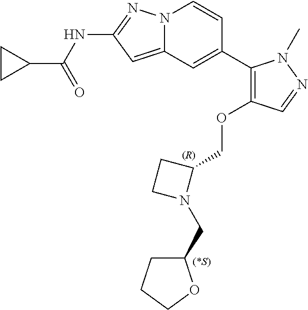

A mixture consisting of (R)—N-(2,6-dimethylpyrimidin-4-yl)-5-(4-((4,4-dimethylpyrrolidin-2-yl)methoxy)-1-methyl-1H-pyrazol-5-yl)pyrazolo[1,5-a]pyridin-2-amine Example 346 (160 mg, 0.269 mmol), tetrahydrofuran-2-carbaldehyde (40.4 mg, 0.404 mmol), NaBH₃CN (50.7 mg, 0.807 mmol) and MeOH (6 mL) was stirred at 30° C. for 2 h. The reaction mixture was filtered and purified by preparative HPLC using a Phenomenex Gemini NX-C18 C18 75×30 mm×3 μm column (eluent: 25% to 55% (v/v) CH3CN and H2O with 0.05% NH3) to afford pure product. The product was suspended in water (10 mL), the mixture frozen using dry ice/ethanol, and then lyophilized to dryness to afford the racemic title compound (115 mg, 95%) as a white solid. (RS)—N-(5-(1-methyl-4-(((2R)-1-((tetrahydrofuran-2-yl)methyl)azetidin-2-yl)methoxy)-1H-pyrazol-5-yl)pyrazolo[1,5-a]pyridin-2-yl)cyclopropanecarboxamide (115 mg, 0.255 mmol) was purified by SFC over DAICEL CHIRALCEL OJ-H 250 mm×30 mm, 5 μm (eluent: 35% to 35% (v/v) supercritical 0.1% NH₃H₂O ETOH). The pure fractions were collected and the volatiles were removed under reduced pressure. The product was suspended in water (10 mL), the mixture frozen using dry ice/ethanol, and then lyophilized to dryness to afford WX_CD48_035_001 (57 mg, 49%) as a white solid and WX_CD48_036_001 (55.5 mg, 48%) as a white solid. MS (ESI): mass calcd. for C₂₄H₃₀N₆O₃, 450.2. m/z found, 451.2 [M+H]⁺. ¹H NMR (400 MHz, DMSO-d6) δ 11.09 (s, 1H), 8.58 (d, J=7.6 Hz, 1H), 7.68 (d, J=1.2 Hz, 1H), 7.44 (s, 1H), 6.96 (dd, J=2.0, 7.2 Hz, 1H), 6.88 (s, 1H), 3.94 (d, J=5.2 Hz, 2H), 3.85 (s, 3H), 3.67-3.55 (m, 2H), 3.49-3.43 (m, 1H), 3.36-3.32 (m, 1H), 3.29-3.22 (m, 1H), 2.86-2.76 (m, 1H), 2.62-2.55 (m, 1H), 2.36-2.29 (m, 1H), 1.99-1.90 (m, 2H), 1.89-1.81 (m, 1H), 1.75-1.56 (m, 3H), 1.42-1.31 (m, 1H), 0.86-0.79 (m, 4H) 1H NMR (400 MHz, CD3OD): 8.44 (d, J=7.6 Hz, 1H), 7.63 (s, 1H), 7.43 (s, 1H), 6.95 (dd, J=2.0, 7.2 Hz, 1H), 6.91 (s, 1H), 4.03 (d, J=5.6 Hz, 2H), 3.88 (s, 3H), 3.78-3.70 (m, 1H), 3.66-3.60 (m, 1H), 3.58-3.50 (m, 2H), 3.40-3.34 (m, 1H), 2.98-2.90 (m, 1H), 2.66 (dd, J=7.2, 12.0 Hz, 1H), 2.41 (dd, J=4.0, 12.4 Hz, 1H), 2.09-1.95 (m, 2H), 1.91-1.77 (m, 2H), 1.76-1.63 (m, 2H), 1.36-1.28 (m, 1H), 1.02-0.95 (m, 2H), 0.93-0.86 (m, 2H)

### Example 354: N-(2,6-dimethylpyrimidin-4-yl)-5-[4-[(2S,3R)-1,2-dimethylpyrrolidin-3-yl]oxy-2-methyl-pyrazol-3-yl]pyrazolo[1,5-a]pyridin-2-amine

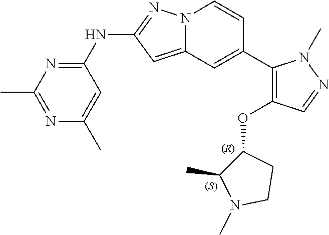

The title compound (59 mg, 4 steps, 9.4%) was prepared as described in Example 9, except 5-(2-((2,6-dimethylpyrimidin-4-yl)amino)pyrazolo[1,5-a]pyridin-5-yl)-1-methyl-1H-pyrazol-4-ol Intermediate 10 was used instead N-(5-(4-hydroxy-1-methyl-1H-pyrazol-5-yl)pyrazolo[1,5-a]pyridin-2-yl)cyclopropanecarboxamide Intermediate 4 and cis-tert-butyl 3-hydroxy-2-methylpyrrolidine-1-carboxylate instead of tert-butyl (S)-2-(hydroxymethyl)azetidine-1-carboxylate. The enantiomers were separated at step B using SFC over DAICEL CHIRALCEL OD-H (250 mm×30 mm, 5 um), (eluent: 35% to 35% (v/v) supercritical CO₂ in EtOH and H₂O with 0.1% NH₃H₂O IPA). The pure fractions were collected and the volatiles were removed under reduced pressure. The product was suspended in water (10 mL), the mixture frozen using dry ice/ethanol, and then lyophilized to dryness to afford P1 (193.9 mg, 43.1%) and P2 (249.9 mg, 55.5%) as a white solid. P1 was taken to step C. MS (ESI): mass calcd. for C₂₃H₂₈N₈O, 432.2. m/z found, 433.2 [M+H]⁺. ¹H NMR (400 MHz, DMSO-d6) δ 10.22 (s, 1H), 8.63 (d, J=7.2 Hz, 1H), 7.68 (d, J=1.2 Hz, 1H), 7.41 (s, 1H), 7.00 (s, 1H), 6.94-6.82 (m, 2H), 4.16-4.09 (m, 1H), 3.85 (s, 3H), 2.90-2.82 (m, 1H), 2.46 (s, 3H), 2.30 (s, 3H), 2.29-2.18 (m, 2H), 2.17 (s, 3H), 2.11-2.00 (m, 1H), 1.69-1.61 (m, 1H), 1.06 (d, J=6.0 Hz, 3H)

### Example 355: N-(2,6-dimethylpyrimidin-4-yl)-5-[2-(trideuteriomethyl)-4-[[(2R)-1-(trideuteriomethyl)azetidin-2-yl]methoxy]pyrazol-3-yl]pyrazolo[1,5-a]pyridin-2-amine

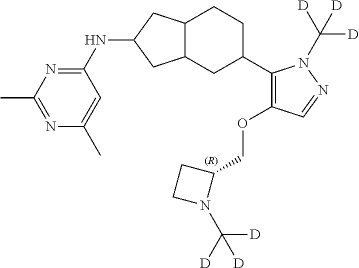

Step A. tert-butyl (R)-2-(((1-((2-(trimethylsilyl)ethoxy)methyl)-1H-pyrazol-4-yl)oxy)methyl)azetidine-1-carboxylate. A mixture consisting of 1-((2-(trimethylsilyl)ethoxy)methyl)-1H-pyrazol-4-ol (1.95 g, 9.10 mmol), (R)-tert-butyl 2-((tosyloxy)methyl)azetidine-1-carboxylate (3.1 g, 9.1 mmol), Cs₂CO₃ (8.9 g, 27 mmol) and DMF (20 mL) was stirred at 80° C. overnight before cooling to room-temperature. The reaction mixture was concentrated to dryness under reduced pressure to give the crude product. To the crude product was added H₂O (50 mL), and then extracted with EtOAc (50 mL×3) and dried over Na₂SO₄, filtered and concentrated under reduce pressure to afford the title compound (4.2 g, 90%) as a brown oil. MS (ESI): mass calcd. for C₁₈H₃₃N₃O₄Si, 383.2. m/z found, 328.2 [M−tBu+H]⁺. 1H NMR (400 MHz, DMSO-d6) δ 7.65-7.62 (m, 1H), 7.34-7.31 (m, 1H), 5.30-5.22 (m, 2H), 4.45-4.34 (m, 1H), 4.13-4.06 (m, 1H), 3.98-3.89 (m, 1H), 3.80-3.67 (m, 2H), 3.50-3.45 (m, 2H), 2.32-2.21 (m, 1H), 2.18-2.03 (m, 1H), 1.35 (s, 9H), 0.83-0.76 (m, 2H), −0.05 (s, 9H)

Step B. tert-butyl (R)-2-(((1H-pyrazol-4-yl)oxy)methyl)azetidine-1-carboxylate. A mixture consisting of (R)-tert-butyl 2-(((1-((2-(trimethylsilyl)ethoxy)methyl)-1H-pyrazol-4-yl)oxy)methyl)azetidine-1-carboxylate from Step A (4.15 g, 8.11 mmol), TBAF (16 mL, 1 M in THF) and THE (16 mL) was stirred overnight at 80° C. The reaction mixture was concentrated under reduced pressure to give the crude product. The product was added H₂O (20 mL), and then extracted with EtOAc (50 mL), the organic layer was washed with H₂O (20 mL×3) and dried over Na₂SO₄, filtered and concentrated under reduce pressure to afford the title compound (2.4 g, crude) as a black oil. MS (ESI): mass calcd. for C₁₂H₁₉N₃O₃, 253.1. m/z found, 198.1 [M−tBu+H]⁺. 1H NMR (400 MHz, DMSO-d6) δ 12.35 (s, 1H), 7.43-7.25 (m, 2H), 4.41-4.33 (m, 1H), 4.11-4.05 (m, 1H), 3.95-3.90 (m, 1H), 3.78-3.68 (m, 2H), 2.29-2.23 (m, 1H), 2.14-2.06 (m, 1H), 1.35 (s, 9H)

Step C. tert-butyl (R)-2-(((1-(methyl-d3)-1H-pyrazol-4-yl)oxy)methyl)azetidine-1-carboxylate. A mixture consisting of (R)-tert-butyl 2-(((1H-pyrazol-4-yl)oxy)methyl)azetidine-1-carboxylate from Step B (2.3 g, 9.1 mmol), CD₃I (2.6 g, 18 mmol), Cs₂CO₃ (5.9 g, 18 mmol) and DMF (30 mL) was stirred at r.t. for 2 h. The reaction mixture was concentrated to dryness under reduced pressure to give the crude product, which was purified by FCC (eluent: petroleum ether: ethyl acetate)=100/0 to 50/50) to yield the title compound (1.25 g, 51% yield) as a yellow oil. MS (ESI): mass calcd. for C₁₃H₁₈D₃N₃O₃, 270.2. m/z found, 215.2 [M−tBu+H]⁺. 1H NMR (400 MHz, DMSO-d6) δ 7.45 (s, 1H), 7.18 (s, 1H), 4.43-4.33 (m, 1H), 4.11-4.01 (m, 1H), 3.95-3.86 (m, 1H), 3.76-3.67 (m, 2H), 2.31-2.20 (m, 1H), 2.16-2.05 (m, 1H), 1.36 (s, 9H).

Step D. tert-butyl (R)-2-(((5-bromo-1-(methyl-d3)-1H-pyrazol-4-yl)oxy)methyl)azetidine-1-carboxylate. (R)-tert-butyl 2-(((1-methyl-1H-pyrazol-4-yl)oxy)methyl)pyrrolidine-1-carboxylate from Step C (1.2 g, 4.4 mmol), NBS (830 mg, 4.66 mmol), and ACN (10 mL) was stirred at room-temperature for 1 h. The reaction mixture was concentrated to dryness under reduced pressure to give the crude product, which was used for the next reaction directly. MS (ESI): mass calcd. for C₁₃H₁₇BrD₃N₃O₃, 348.1. m/z found, 295.0 [M−tBu+H]⁺.

Step E. tert-butyl (R)-2-(((5-(2-aminopyrazolo[1,5-a]pyridin-5-yl)-1-(methyl-d3)-1H-pyrazol-4-yl)oxy)methyl)azetidine-1-carboxylate. A mixture consisting of (R)-tert-butyl 2-(((5-bromo-1-methyl-1H-pyrazol-4-yl)oxy)methyl)azetidine-1-carboxylate from Step D (1.95 g, 5.58 mmol), 5-(4,4,5,5-tetramethyl-1,3,2-dioxaborolan-2-yl)pyrazolo[1,5-a]pyridin-2-amine (2.17 g, 8.38 mmol), Pd(dppf)Cl₂ (409 mg, 0.558 mmol), K₃PO₄ (3.56 g, 16.8 mmol), and 1,4-dioxane/H₂O (30 mL, 4:1) under Ar atmosphere was stirred 2 h at 90° C. The reaction mixture was concentrated to dryness under reduced pressure to give the crude product, which was purified by FCC (eluent: petroleum ether:ethyl acetate)=100/0 to 0/100) to yield the title compound of P1 (1.1 g, 49%) as a yellow oil. MS (ESI): mass calcd. for C₂₀H₂₃D₃N₆O₃, 401.2. m/z found, 402.3 [M+H]⁺. 1H NMR (400 MHz, DMSO-d6) δ 8.32 (d, J=7.2 Hz, 1H), 7.43 (s, 1H), 7.40 (s, 1H), 6.69 (d, J=7.2 Hz, 1H), 5.69 (s, 1H), 5.36 (s, 2H), 4.43-4.31 (m, 1H), 4.27-4.15 (m, 1H), 4.01-3.96 (m, 1H), 3.73-3.60 (m, 2H), 2.31-2.20 (m, 1H), 2.15-2.06 (m, 1H), 1.29 (s, 9H).

Step F. tert-butyl (R)-2-(((5-(2-((2,6-dimethylpyrimidin-4-yl)amino)pyrazolo[1,5-a]pyridin-5-yl)-1-(methyl-d3)-1H-pyrazol-4-yl)oxy)methyl)azetidine-1-carboxylate. A mixture consisting of (R)-tert-butyl 2-(((5-(2-aminopyrazolo[1,5-a]pyridin-5-yl)-1-methyl-1H-pyrazol-4-yl)oxy)methyl)azetidine-1-carboxylate from Step E (500 mg, 1.25 mmol), 4-chloro-2,6-dimethylpyrimidine (213 mg, 1.49 mmol), Brettphos-Pd-G3 (203 mg, 0.224 mmol), Brettphos (267 mg, 0.498 mmol), Cs₂CO₃ (1.22 g, 3.74 mmol) and 1,4-dioxane (10 mL) was purged with Ar for 2 min. The resultant mixture was stirred at 50° C. for 6 h before cooling to room temperature. The reaction mixture was concentrated to dryness under reduced pressure to give the crude product, which was purified by FCC (eluent: petroleum ether:ethyl acetate; 100/0 to 0/100) to yield the title compound (470 mg, 74%) as a light yellow solid. MS (ESI): mass calcd. for C₂₆H₂₉D₃N₈O₃, 507.3. m/z found, 508.3 [M+H]⁺. 1H NMR (400 MHz, DMSO-d6) δ 10.22 (s, 1H), 8.60 (d, J=7.2 Hz, 1H), 7.72 (s, 1H), 7.46 (s, 1H), 7.00 (s, 1H), 6.96 (d, J=7.6 Hz, 1H), 6.88 (s, 1H), 4.41-4.34 (m, 1H), 4.27-4.20 (m, 1H), 3.75-3.61 (m, 2H), 3.40-3.37 (m, 1H), 2.46 (s, 3H), 2.31-2.29 (m, 3H), 2.28-2.21 (m, 1H), 2.15-2.06 (m, 1H), 1.29 (s, 9H)

Step G. (R)-5-(4-(azetidin-2-ylmethoxy)-1-(methyl-d3)-1H-pyrazol-5-yl)-N-(2,6-dimethylpyrimidin-4-yl)pyrazolo[1,5-a]pyridin-2-amine. TFA (1999 mg, 17.53 mmol) from Step F was added to a solution consisting of (R)-tert-butyl 2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1 carboxylate (445 mg, 0.877 mmol) and DCM (2 mL). The resultant mixture was stirred at room-temperature for 0.5 h. The reaction mixture concentrated to dryness under reduced pressure to give the crude product, then adjusted pH=7 with saturated aqueous NaHCO₃, The product was suspended in water (10 mL), the mixture frozen using dry ice/ethanol, and then lyophilized to dryness to afford the title compound as a white solid (320 mg, crude). MS (ESI): mass calcd. for C₂₁H₂₁D₃N₈O, 407.2. m/z found, 408.2 [M+H]⁺.

Step H. (R)—N-(2,6-dimethylpyrimidin-4-yl)-5-(1-(methyl-d3)-4-((1-(methyl-d3)azetidin-2-yl)methoxy)-1H-pyrazol-5-yl)pyrazolo[1,5-a]pyridin-2-amine. A solution consisting of (R)-5-(4-(azetidin-2-ylmethoxy)-1-methyl-1H-pyrazol-5-yl)-N-(2,6-dimethylpyrimidin-4-yl)pyrazolo[1,5-a]pyridin-2-amine from Step G (135 mg, 0.331 mmol), paraformaldehyde-d2 (48 mg, 0.50 mmol), NaBD₃CN (33 mg, 0.50 mmol) in MeOD (2 mL) was stirred for 0.5 hs at 25° C. The mixture was purified by preparative HPLC with a Welch Xtimate 150×25 mm×5 um column (eluent: 21% to 51% (v/v) CH₃CN and H₂O with 0.04% NH₃) to afford product. The product was suspended in water (10 mL), the mixture frozen using dry ice/EtOH, and then lyophilized to dryness to afford P1 (52 mg, 37%) as a white solid. MS (ESI): mass calcd. for C₂₂H₂₀D₆N₈O, 424.3. m/z found, 425.2 [M+H]⁺. ¹H NMR (400 MHz, DMSO-d6) δ 10.21 (s, 1H), 8.62 (d, J=7.2 Hz, 1H), 7.69 (d, J=0.8 Hz, 1H), 7.45 (s, 1H), 7.00 (s, 1H), 6.93 (dd, J=2.09, 7.2 Hz, 1H), 6.87 (s, 1H), 4.00-3.86 (m, 2H), 3.31-3.25 (m, 2H), 2.80-2.70 (m, 1H), 2.46 (s, 3H), 2.30 (s, 3H), 2.00-1.91 (m, 1H), 1.90-1.78 (m, 1H) 1H NMR (400 MHz, CDCl₃) δ 8.40 (d, J=7.2 Hz, 1H), 7.65 (s, 1H), 7.47 (d, J=1.2 Hz, 1H), 7.35 (s, 1H), 7.06 (s, 1H), 6.86 (dd, J=2.0, 7.2 Hz, 1H), 6.59 (s, 1H), 4.06-3.93 (m, 2H), 3.49-3.41 (m, 1H), 3.41-3.32 (m, 1H), 2.93-2.80 (m, 1H), 2.61 (s, 3H), 2.45 (s, 3H), 2.08-2.00 (m, 2H)

### Example 356: (1*R)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol

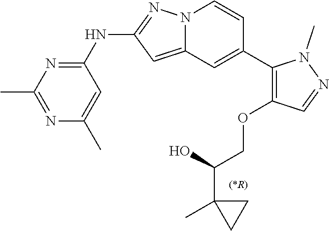 [SMILES: Cc1cc(Nc2cc3cc(-c4c(OC[C@H](O)C5(C)CC5)cnn4C)ccn3n2)nc(C)n1]

Step A. 2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethan-1-one. A solution of 5-(2-((2,6-dimethylpyrimidin-4-yl)amino)pyrazolo[1,5-a]pyridin-5-yl)-1-methyl-1H-pyrazol-4-ol, Intermediate 10, (500 mg, 1.49 mmol), 2-chloro-1-(1-methylcyclopropyl)ethanone (197.7 mg, 1.491 mmol) in DMF (15 mL) was added Cs₂CO₃ (971.5 mg, 2.982 mmol). The reaction mixture was stirred at r.t for 2 hr. The mixture was concentrated. The reaction mixture was poured into H₂O (150 mL) and extracted with EtOAc (50 mL×3). The combined organic extracts were washed with brine (200 mL), dried over anhydrous Na₂SO₄, filtered, and concentrated to dryness under reduced pressure to give crude product. The crude product was purified by FCC (MeOH:DCM=1:10 to 1:10) to afford compound (510 mg, 66.76%) as yellow solid. MS (ESI): mass calcd. for C₂₃H₂₅N₇O₂, 431.2. m/z found, 432.2 [M+H]⁺.

Step B. (R)-2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethan-1-ol. To a solution of 2-((5-(2-((2,6-dimethylpyrimidin-4-yl)amino)pyrazolo[1,5-a]pyridin-5-yl)-1-methyl-1H-pyrazol-4-yl)oxy)-1-(1-methylcyclopropyl)ethenone, Step A, (460 mg, 1.07 mmol) in MeOH (10 mL) was added NaBH₄ (80.7 mg, 2.13 mmol) at 0° C. The mixture was stirred at r.t for 1 hrs. The reaction mixture was filtered and the filtrate was purified by preparative HPLC using a YMC-Triart Prep C18 250×50 mm×10 um column (eluent: 25% to 55% (v/v) CH₃CN and H₂O with 0.04% NH₃) to afford pure product. The product was suspended in water (10 mL), the mixture frozen using dry ice/ethanol, and then lyophilized to dryness to afford the racemic form of the title compound (210 mg, 45%) as a yellow oil. The enantiomers were separated via SFC (Column: DAICEL CHIRALCEL OJ-H(250 mm*30 mm, 5 um), Condition: 0.1% NH₃H₂O ETOH, Begin B: 25%, End B 25%. FlowRate: 60 ml/min) to give the titled compound P1 as a white solid (73.1 mg, 34.8%) and the corresponding enantiomer P2 as a white solid (84.2 mg, 40.1%). MS (ESI): mass calcd. for C₂₃H₂₇N₇O₂, 433.2. m/z found, 434.1 [M+H]⁺. ¹H NMR (400 MHz, CDCl₃) δ 8.37 (d, J=7.2 Hz, 1H), 7.53 (br. s., 1H), 7.45 (s, 1H), 7.38 (s, 1H), 7.02 (s, 1H), 6.81 (dd, J=1.2, 6.8 Hz, 1H), 6.59 (s, 1H), 4.14-4.08 (m, 1H), 4.05-3.98 (m, 1H), 3.91 (s, 3H), 3.34-3.27 (m, 1H), 2.60 (s, 3H), 2.44 (s, 3H), 2.38 (br. s., 1H), 1.07 (s, 3H), 0.54-0.45 (m, 2H), 0.38-0.32 (m, 2H)

### Example 357: *S-1-methylcyclopropyl-2-[5-[2-[(2,6-dimethylpyrimidin-4-yl)amino]pyrazolo[1,5-a]pyridin-5-yl]-1-methyl-pyrazol-4-yl]oxy-ethanol

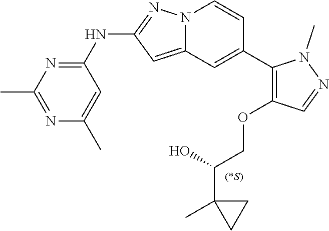 [SMILES: Cc1cc(Nc2cc3cc(-c4c(OC[C@@H](O)C5(C)CC5)cnn4C)ccn3n2)nc(C)n1]

The title compound (84 mg, 40%) was prepared as described in Example 356, where the title compound was collected after SFC. MS (ESI): mass calcd. for C₂₂H₂₅N₇O₂, 419.2. m/z found, 420.1 [M+H]⁺. ¹H NMR (400 MHz, CDCl₃) δ 8.37 (d, J=7.2 Hz, 1H), 7.49 (br. s., 1H), 7.45 (d, J=1.2 Hz, 1H), 7.38 (s, 1H), 7.01 (s, 1H), 6.81 (dd, J=2.0, 7.2 Hz, 1H), 6.59 (s, 1H), 4.15-4.07 (m, 1H), 4.03-3.95 (m, 1H), 3.91 (s, 3H), 3.30-3.21 (m, 1H), 2.60 (s, 3H), 2.54-2.37 (m, 4H), 0.99-0.88 (m, 1H), 0.64-0.49 (m, 2H), 0.44-0.35 (m, 1H), 0.32-0.23 (m, 1H).

### Example 358: *S—N-(2,6-dimethylpyrimidin-4-yl)-5-[2-methyl-4-(7-oxaspiro[3.4]octan-6-ylmethoxy)pyrazol-3-yl]pyrazolo[1,5-a]pyridin-2-amine

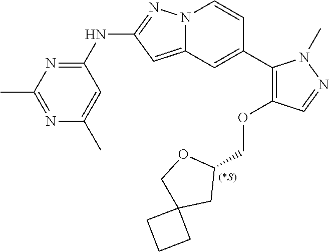 [SMILES: Cc1cc(Nc2cc3cc(-c4c(OC[C@@H]5CC6(CCC6)CO5)cnn4C)ccn3n2)nc(C)n1]

The title compound (31 mg, 9.2%) was prepared as described in Example 356 Step A, where (RS)-6-oxaspiro[3.4]octan-7-ylmethyl 4-methylbenzenesulfonate was used instead of 2-chloro-1-(1-methylcyclopropyl)ethenone to obtained the racemic mixture of the titled compound. The racemate was separated by SFC over DAICEL CHIRALPAK AD 250 mm×50 mm×10 μm (eluent: 55% to 55% (v/v) supercritical CO₂ in EtOH and H₂O with 0.1% NH₃) and concentrated under reduced pressure. The product was suspended in water (10 mL), the mixture frozen using dry ice/acetone, and then lyophilized to dryness to afford the corresponding enantiomer P1 (31.8 mg, 40%) as a white solid and the titled compound P2 (31.6 mg, 39%) as a white solid. MS (ESI): mass calcd. for C₂₅H₂₉N₇O₂, 459.2. m/z found, 460.2 [M+H]⁺. ¹H NMR (400 MHz, DMSO-d6) δ 10.23 (s, 1H), 8.62 (d, J=7.2 Hz, 1H), 7.69 (d, J=0.8 Hz, 1H), 7.45 (s, 1H), 6.99 (s, 1H), 6.93 (dd, J=2.0, 7.2 Hz, 1H), 6.89 (s, 1H), 4.17-4.05 (m, 1H), 3.96-3.88 (m, 2H), 3.87 (s, 3H), 3.63 (s, 2H), 2.46 (s, 3H), 2.30 (s, 3H), 2.08-2.02 (m, 1H), 1.98-1.89 (m, 4H), 1.85-1.75 (m, 2H), 1.74-1.68 (m, 1H)

### Example 359: N-(5,6-dimethylpyrazin-2-yl)-5-[4-[[(2S)-1-ethylazetidin-2-yl]methoxy]-2-methyl-pyrazol-3-yl]pyrazolo[1,5-a]pyridin-2-amine

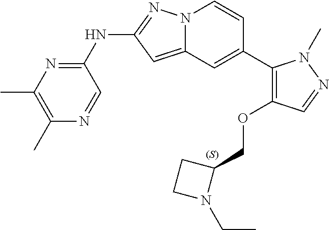

The title compound (36.8 mg, 3 steps, 17%) was prepared as described in Example 338, where enantiomer of Intermediate 6 was used. MS (ESI): mass calcd. for C₂₃H₂₈N₈O, 432.2. m/z found, 433.1 [M+H]⁺. ¹H NMR (400 MHz, DMSO-d6) δ 9.94 (s, 1H), 8.58 (d, J=7.2 Hz, 1H), 8.43 (s, 1H), 7.65 (d, J=1.2 Hz, 1H), 7.44 (s, 1H), 6.90 (dd, J=2.0, 7.2 Hz, 1H), 6.85 (s, 1H), 3.95-3.91 (m, 2H), 3.87 (s, 3H), 3.33-3.20 (m, 2H), 2.70-2.62 (m, 1H), 2.61-2.54 (m, 1H), 2.43 (s, 3H), 2.38 (s, 3H), 2.29-2.19 (m, 1H), 1.99-1.90 (m, 1H), 1.89-1.78 (m, 1H), 0.83 (t, J=7.6 Hz, 3H)

### Example 360: N-[5-[4-(azetidin-3-yloxymethyl)-2-methyl-pyrazol-3-yl]pyrazolo[1,5-a]pyridin-2-yl]cyclopropanecarboxamide

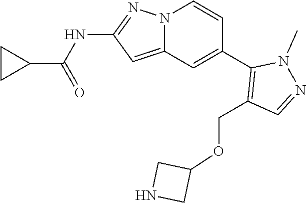 [SMILES: Cn1ncc(COC2CNC2)c1-c1ccn2nc(NC(=O)C3CC3)cc2c1]

Step A. tert-butyl 3-((5-bromo-1-methyl-1H-pyrazol-4-yl)methoxy)azetidine-1-carboxylate. (5-bromo-1-methyl-1H-pyrazol-4-yl)methanol (355 mg, 1.86 mmol) was dissolved in DMA (3 mL) and treated with tert-butyl 3-((methylsulfonyl)oxy)azetidine-1-carboxylate (514 mg, 2.05 mmol). NaH (60% dispersion in mineral oil) (148.7 mg, 3.72 mmol) was added in a single portion and the reaction mixture was stirred at room temperature for 1 h. The reaction mixture was quenched with the addition of saturated aqueous sodium bicarbonate (20 mL) and extracted 3×50 mL EtOAc. The combined organic layers were dried (MgSO₄) and concentrated. The residue was purified FCC (SiO₂, 30-100% EtOAc/hexanes). Pure Fractions were combined and concentrated to give the titled compound as a clear oil that solidified upon standing.

Step B. tert-butyl 3-((5-(2-(cyclopropanecarboxamido)pyrazolo[1,5-a]pyridin-5-yl)-1-methyl-1H-pyrazol-4-yl)methoxy)azetidine-1-carboxylate. To a microwave vial was added a stir bar, Intermediate 3 (206 mg, 0.63 mmol), tert-butyl 3-((5-bromo-1-methyl-1H-pyrazol-4-yl)methoxy)azetidine-1-carboxylate from Step A (218 mg, 0.63 mmol), and Ruphos-G2 (24.53 mg, 0.032 mmol). The flask treated with 1,4-dioxane (1.5 mL) and 0.5 M aqueous potassium phosphate solution (2 mL), then degassed for 2 minutes under vacuum with sonication, then vented to nitrogen. The flask was heated in the microwave for 1 h at 100° C. The reaction mixture was extracted 2×20 mL DCM, then the combined aqueous layers were dried (MgSO₄) and concentrated. The residue was purified (FCC) 40-70% ETOAc/hexanes. Fractions containing titled compound were combined and concentrated, then lyophilized to afford an off-white solid (139 mg, 47%). MS (ESI): mass calcd. for C₂₄H₃₀N₆O₄, 466.2. m/z found, 267.2 [M−Boc+H]⁺ and 489.2 [M+Na⁺].

Step C. N-(5-(4-((azetidin-3-yloxy)methyl)-1-methyl-1H-pyrazol-5-yl)pyrazolo[1,5-a]pyridin-2-yl)cyclopropanecarboxamide. A 25 mL round bottom flask containing tert-butyl 3-((5-(2-(cyclopropanecarboxamido)pyrazolo[1,5-a]pyridin-5-yl)-1-methyl-1H-pyrazol-4-yl)methoxy)azetidine-1-carboxylate (125 mg, 0.268 mmol) from Step B and a stir bar was treated with 4 mL TFA and stirred at room temperature. The TFA was evaporated in a rotary evaporator. The residue was dissolved in 10 mL DCM and extracted with 10 mL saturated aqueous sodium bicarbonate. The organic layer was separated, and the aqueous layer was extracted again with 2×20 mL DCM. The combined organics were dried (MgSO₄). The residue was purified via reverse phase (HPLC, NH₄OH-Water-acetonitrile). The fractions containing titled compound were combined and concentrated. The residue was lyophilized from 1:1 CH₃CN-water to give P1 as a white solid (13.1 mg, 13.3%). MS (ESI): mass calcd. for C₁₉H₂₂N₆O₂, 366.2. m/z found, 367.2 [M+H]⁺. ¹H NMR (400 MHz, DMSO-d6) δ 11.12 (s, 1H), 8.65 (d, J=7.1 Hz, 1H), 7.71 (s, 1H), 7.55 (s, 1H), 6.97-6.89 (m, 2H), 4.32-4.19 (m, 1H), 4.17 (s, 2H), 3.82 (s, 3H), 3.6-3.3 (m, 4H, partially obscured), 1.99-1.87 (m, 1H), 0.89-0.76 (m, J=5.7 Hz, 4H).

### Example 361: N-[5-[2-methyl-4-[(1-methylazetidin-3-yl)oxymethyl]pyrazol-3-yl]pyrazolo[1,5-a]pyridin-2-yl]cyclopropanecarboxamide

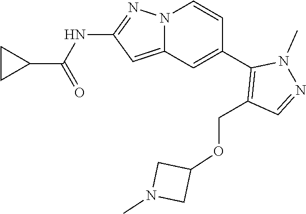 [SMILES: CN1CC(OCc2cnn(C)c2-c2ccn3nc(NC(=O)C4CC4)cc3c2)C1]

The title compound (24 mg, 30%) was prepared as described in Example 334, where para-formaldehyde was used instead of acetaldehyde and N-[5-[4-(azetidin-3-yloxymethyl)-2-methyl-pyrazol-3-yl]pyrazolo[1,5-a]pyridin-2-yl]cyclopropanecarboxamideI Example 360 instead of (R)-5-(4-(azetidin-2-ylmethoxy)-1-methyl-1H-pyrazol-5-yl)-N-(5,6-dimethylpyridazin-3-yl)pyrazolo[1,5-a]pyridin-2-amine. MS (ESI): mass calcd. for C₂₃H₂₈N₈O, 432.2. m/z found, 433.1 [M+H]⁺. ¹H NMR (400 MHz, DMSOMS (ESI): mass calcd. for C₂₀H₂₄N₆O₂, 380.2. m/z found, 381.2 [M+H]⁺. ¹H NMR (500 MHz, Methanol-d4) δ 8.50 (dt, J=7.2, 1.0 Hz, 1H), 7.65 (dd, J=2.0, 1.0 Hz, 1H), 7.58 (s, 1H), 6.95 (s, 1H), 6.88 (dd, J=7.1, 1.9 Hz, 1H), 4.27 (s, 2H), 4.10 (p, J=5.9 Hz, 1H), 3.85 (s, 3H), 3.58-3.47 (m, 2H), 3.00-2.87 (m, 2H), 2.31 (s, 3H), 1.87 (tt, J=8.2, 4.5 Hz, 1H), 0.99 (dt, J=4.5, 3.2 Hz, 2H), 0.90 (dt, J=8.1, 3.3 Hz, 2H).

### Example 362: N-[5-[2-methyl-4-[[(3R)-1-methylpyrrolidin-3-yl]oxymethyl]pyrazol-3-yl]pyrazolo[1,5-a]pyridin-2-yl]cyclopropanecarboxamide

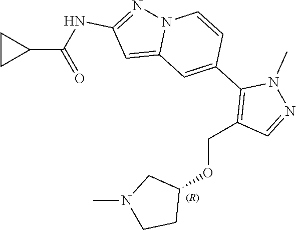

Step A. tert-butyl (R)-3-((5-bromo-1-methyl-1H-pyrazol-4-yl)methoxy)pyrrolidine-1-carboxylate. To a 25 mL flask was added tert-butyl (R)-3-hydroxypyrrolidine-1-carboxylate, (536.315 mg, 2.864 mmol) and DMF (7.208 mL) and cooled to 0 C. Then, NaH (60% dispersion 5 in mineral oil) (171.844 mg, 4.297 mmol) was added. The mixture was stirred for 5 min before syringe into a flask containing 5-bromo-4-(chloromethyl)-1-methyl-1H-pyrazole (300 mg, 1.432 mmol). The reaction was heated to 80 C for 4 h in microwave. The crude was quenched with H₂O and then diluted with EtOAc. The organics were washed 2× with H₂O and concentrated down in the presence of silica gel. The crude was flashed with 20-100% EtOAc:hex. The pure fraction was collected and concentrated down under vacuo to afford the titled compound (350 mg, 68%).

Step B. (R)—N-(5-(1-methyl-4-((pyrrolidin-3-yloxy)methyl)-1H-pyrazol-5-yl)pyrazolo[1,5-a]pyridin-2-yl)cyclopropanecarboxamide. To a microwave vial was added a stir bar, Intermediate 3 (381.457 mg, 1.166 mmol) and Ruphos-G2 [1375325-68-0] (31.535 mg, 0.0406 mmol). The flask treated with a solution of tert-butyl (R)-3-((5-bromo-1-methyl-1H-pyrazol-4-yl)methoxy)pyrrolidine-1-carboxylate from Step A (350 mg, 0.972 mmol) and 1,4-dioxane (2.699 mL, 0.3 M, 0.81 mmol) and then followed with tribasic potassium phosphate 0.5 M in water (1.781 mL) then degassed by vacuum and back-filled with N₂ 3×. The flask was heated in the microwave for 1 h at 90° C. The residue was purified via FCC using 30-100% EtOAc:hex. This material was used in the next step.

To the clear oil from the previous step was added DCM (5.186 mL, 1.326 g/mL, 80.962 mmol) and then TFA (3.098 mL, 1.49 g/mL, 40.481 mmol). The reaction was stirred for 1 h. The crude was concentrated down under vacuum. The crude was dissolved with 0.5 mL of 2M NH₃ in MeOH and purified via prep. HPLC using 10-30% ACN-H₂O with 20 mM of NH₄OH as modifier. MS (ESI): mass calcd. for C₂₁H₂₆N₆O₂, 394.2. m/z found, 395.1 [M+H]⁺. ¹H NMR (500 MHz, Methanol-d4) δ 8.49 (d, J=7.0 Hz, 1H), 7.65 (s, 1H), 7.57 (d, J=1.7 Hz, 1H), 6.94 (s, 1H), 6.88 (dt, J=7.2, 2.0 Hz, 1H), 4.28 (t, J=2.3 Hz, 2H), 4.10 (s, 1H), 3.85 (d, J=1.7 Hz, 3H), 2.73-2.62 (m, 2H), 2.57 (d, J=10.8 Hz, 1H), 2.43 (q, J=8.1 Hz, 1H), 2.31 (d, J=1.8 Hz, 3H), 2.06 (dq, J=14.0, 7.0 Hz, 1H), 1.87 (s, 1H), 1.80 (s, 1H), 1.04-0.97 (m, 2H), 0.95-0.84 (m, 2H).

### Example 363: N-[5-[2-methyl-4-[[(3S)-pyrrolidin-3-yl]oxymethyl]pyrazol-3-yl]pyrazolo[1,5-a]pyridin-2-yl]cyclopropanecarboxamide

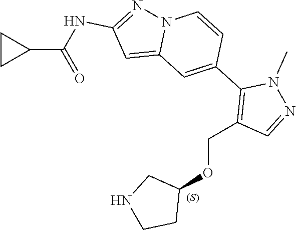

The title compound (24 mg, 30%) was prepared as described in Example 360 where tert-butyl (S)-3-hydroxypyrrolidine-1-carboxylate was used instead of tert-butyl (R)-3-hydroxypyrrolidine-1-carboxylate. MS (ESI): mass calcd. for C₂₀H₂₄N₆O₂, 380.2. m/z found, 381.1 [M+H]⁺. ¹H NMR (400 MHz, Chloroform-d) δ 8.42 (d, J=7.1 Hz, 1H), 8.29 (s, 1H), 7.69 (d, J=15.1 Hz, 1H), 7.58 (s, 1H), 7.52 (s, 1H), 7.10 (s, 1H), 6.80 (dd, J=19.4, 6.7 Hz, 1H), 4.40-4.25 (m, 2H), 4.16-4.05 (m, 2H), 3.97 (d, J=11.0 Hz, 3H), 3.53-3.32 (m, 4H), 1.96 (tt, J=8.7, 3.9 Hz, 2H), 1.63 (s, 1H), 1.25-1.15 (m, 2H), 1.02-0.90 (m, 2H). 1H NMR (500 MHz, Methanol-d4) δ 8.50 (dt, J=7.1, 1.0 Hz, 1H), 7.64 (dd, J=2.0, 1.0 Hz, 1H), 7.59 (s, 1H), 6.95 (s, 1H), 6.87 (dd, J=7.1, 1.9 Hz, 1H), 4.51-4.33 (m, 2H), 3.85 (s, 3H), 3.49 (ddd, J=15.0, 10.4, 4.5 Hz, 1H), 3.00 (dd, J=10.7, 4.4 Hz, 1H), 2.92 (dd, J=11.2, 3.8 Hz, 1H), 2.38 (ddd, J=16.4, 7.3, 3.3 Hz, 1H), 2.28 (s, 3H), 2.17 (d, J=12.1 Hz, 1H), 1.87 (t, J=11.3 Hz, 2H), 1.71 (t, J=10.5 Hz, 1H), 1.11 (q, J=12.0 Hz, 1H), 0.99 (dt, J=4.6, 3.1 Hz, 2H), 0.90 (dt, J=8.1, 3.3 Hz, 2H).

### Example 364: Rac-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

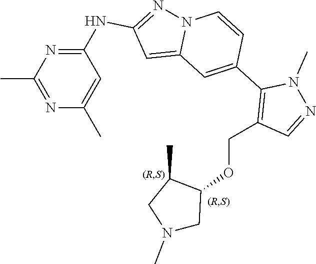

Step A and B. Trans-Rac-tert-butyl 3-((5-(2-aminopyrazolo[1,5-a]pyridin-5-yl)-1-methyl-1H-pyrazol-4-yl)methoxy)-4-methylpyrrolidine-1-carboxylate. The title compound (500 mg, 52%) was prepared as described in Example 360 where Rac-trans-(3S,4R)-1,4-dimethylpyrrolidin-3-ol was used instead of tert-butyl (R)-3-hydroxypyrrolidine-1-carboxylate and Intermediate 5 was used instead of Intermediate 3. MS (ESI): mass calcd. for C₂₂H₃₀N₆O₃, 426.2. m/z found, 427.3 [M+H]⁺.

Step C. N-(2,6-dimethylpyrimidin-4-yl)-5-(1-methyl-4-((((3R,4S)-4-methylpyrrolidin-3-yl)oxy)methyl)-1H-pyrazol-5-yl)pyrazolo[1,5-a]pyridin-2-amine. To a microwave vial was added tBuBrettPhos Pd G3 [1536473-72-9] (20.033 mg, 0.0234 mmol) and Cs₂CO₃ (152.781 mg, 0.469 mmol). The solid mixture was evacuated and back-filled with N2 3×. Then, a solution of CIS-tert-butyl-3-((5-(2-aminopyrazolo[1,5-a]pyridin-5-yl)-1-methyl-1H-pyrazol-4-yl)methoxy)-4-methylpyrrolidine-1-carboxylate (100 mg, 0.234 mmol) and 1,4-dioxane (1 mL, 1.033 g/mL, 11.723 mmol) was added. The reaction mixture was evacuated and back-filled with N2 3× more. After 1 h, 90° C. in heating block. To the reaction mixture was added more tBuBrettPhos Pd G3 [1536473-72-9] (20.033 mg, 0.0234 mmol) and evacuated and backfilled with N2 5×. I evacuated the N2 for 15-30 seconds each time. The reaction was heated to 80° C. in heating block. The crude reaction mixture was diluted with EtOAc and H2O. The layers were separated. The organic layer was washed with H2O 3×. The organic was concn down under vacuo. The residue was flashed with 50-100% EtOAc:Hex. The pure fractions were collected.

To the residue was added 4:1 DCM:TFA. The reaction was stirred for 1 h. The solution was concentrated down under vacuo to afford the titled compound (50 mg, 49%). MS (ESI): mass calcd. for C₂₃H₂₈N₈O, 432.2. m/z found, 433.2 [M+H]⁺.

Step D. N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine. To a solution of N-(2,6-dimethylpyrimidin-4-yl)-5-(1-methyl-4-((((3R,4S)-4-methylpyrrolidin-3-yl)oxy)methyl)-1H-pyrazol-5-yl)pyrazolo[1,5-a]pyridin-2-amine from Step B (25 mg, 0.0457 mmol) and MeOH (0.185 mL, 0.791 g/mL, 4.574 mmol) was added H(CHO)n (0.0033 g, 0.0366 mmol) and NaCNBH3 [25895-60-7] (0.00862 g, 0.137 mmol). After 1 h stirring, LCMS showed mostly SM. The reaction was heated to 40 C and stirred for 120 min. LCMS showed mostly desired material. The crude was diluted with MeOH and filter. The solution was purified via prep HPLC using 10-70% ACN-H2O with 20 mM of aq. NH₄OH as modifier. The pure fractions were collected, froze, and lyophilized to a white powder (4.5 mg, 22%). MS (ESI): mass calcd. for C₂₄H₃₀N₈O, 446.3. m/z found, 447.2 [M+H]⁺. ¹H NMR (500 MHz, Methanol-d4) δ 8.55 (dt, J=7.1, 0.9 Hz, 1H), 7.69 (dd, J=1.9, 0.9 Hz, 1H), 7.61 (s, 1H), 7.06 (s, 1H), 6.97 (s, 1H), 6.89 (dd, J=7.1, 1.9 Hz, 1H), 4.40 (d, J=11.5 Hz, 1H), 4.31 (d, J=11.5 Hz, 1H), 3.95 (td, J=5.9, 3.7 Hz, 1H), 3.89 (s, 3H), 3.02 (dd, J=10.8, 5.5 Hz, 1H), 2.85 (dd, J=8.7, 6.7 Hz, 1H), 2.56 (s, 3H), 2.50 (dd, J=10.8, 3.7 Hz, 1H), 2.41 (d, J=0.6 Hz, 3H), 2.34 (s, 3H), 2.32-2.21 (m, 2H), 0.99 (d, J=6.8 Hz, 3H).

### Example 365: Trans-*R, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

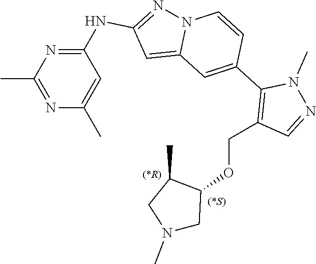

The title compound (5.4 mg, 26%) was prepared as described in Example 364. The enantiomers were separated via chiral SFC using AS-H (2×25 cm) column as immobile phase and 25% MeOH (0.1% DEA)/CO₂, 100 bar, 60 mL/min. MS (ESI): mass calcd. for C₂₄H₃₀N₈O, 446.3. m/z found, 447.2 [M+H]⁺. ¹H NMR (400 MHz, Methanol-d4) δ 8.43 (dt, J=7.1, 0.9 Hz, 1H), 7.56 (dd, J=1.9, 0.9 Hz, 1H), 7.49 (s, 1H), 6.93 (s, 1H), 6.85 (s, 1H), 6.77 (dd, J=7.1, 2.0 Hz, 1H), 4.28 (d, J=11.5 Hz, 1H), 4.19 (d, J=11.5 Hz, 1H), 3.83 (td, J=5.7, 3.5 Hz, 1H), 3.77 (s, 3H), 2.91 (dd, J=10.9, 5.4 Hz, 1H), 2.81-2.68 (m, 1H), 2.43 (s, 4H), 2.29 (d, J=0.6 Hz, 3H), 2.23 (s, 3H), 2.21-2.10 (m, 2H), 0.87 (d, J=6.6 Hz, 3H).

### Example 366: Trans-*S, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

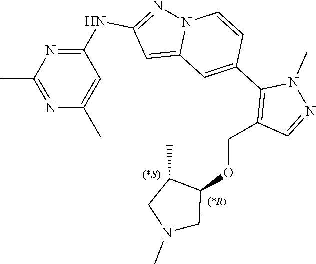

The title compound (8.3 mg, 40%) was prepared as described in Example 364. The enantiomers were separated via chiral SFC using AS-H (2×25 cm) column as immobile phase and 25% MeOH (0.1% DEA)/CO₂, 100 bar, 60 mL/min. MS (ESI): mass calcd. for C₂₄H₃₀N₈O, 446.3. m/z found, 447.3 [M+H]⁺. ¹H NMR (400 MHz, Methanol-d4) δ 8.43 (dt, J=7.2, 0.9 Hz, 1H), 7.56 (dd, J=2.0, 0.9 Hz, 1H), 7.49 (s, 1H), 6.93 (s, 1H), 6.86 (d, J=3.8 Hz, 1H), 6.76 (dd, J=7.2, 1.9 Hz, 1H), 4.36-4.25 (m, 1H), 4.20 (d, J=11.5 Hz, 1H), 3.85 (q, J=5.2 Hz, 1H), 3.77 (s, 3H), 2.94 (dd, J=11.0, 5.2 Hz, 1H), 2.87-2.74 (m, 1H), 2.51 (dd, J=11.0, 3.3 Hz, 1H), 2.43 (s, 3H), 2.29 (s, 6H), 2.25-2.14 (m, 2H), 0.91-0.84 (m, 3H).

### Example 367: (rac-cis)-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

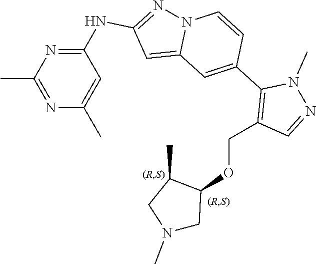

The title compound (5.4 mg, 2.2%) was prepared as described in Example 364 where Rac-cis-(3S,4R)-1,4-dimethylpyrrolidin-3-ol. MS (ESI): mass calcd. for C₂₄H₃₀N₈O, 446.3. m/z found, 447.3 [M+H]⁺. ¹H NMR (400 MHz, Methanol-d4) δ 8.43 (dt, J=7.2, 1.0 Hz, 1H), 7.57 (dd, J=1.9, 0.9 Hz, 1H), 7.49 (s, 1H), 6.94 (s, 1H), 6.85 (s, 1H), 6.77 (dd, J=7.1, 1.9 Hz, 1H), 4.28 (d, J=11.5 Hz, 1H), 4.19 (d, J=11.5 Hz, 1H), 3.84 (td, J=5.9, 3.7 Hz, 1H), 3.77 (s, 3H), 2.90 (dd, J=10.8, 5.5 Hz, 1H), 2.77-2.69 (m, 1H), 2.44 (s, 3H), 2.38 (dd, J=10.8, 3.7 Hz, 1H), 2.29 (d, J=0.6 Hz, 3H), 2.22 (s, 3H), 2.20-2.09 (m, 2H), 0.87 (d, J=6.7 Hz, 3H).

### Example 368: CIS—*R, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

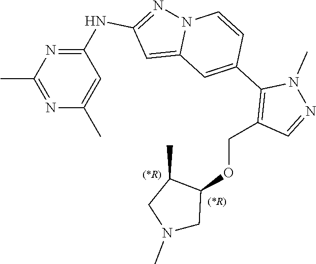

The title compound (5.4 mg, 2.2%) was prepared as described in Example 364 where Rac-cis-(3S,4R)-1,4-dimethylpyrrolidin-3-ol. The enantiomers were separated via chiral SFC using AS-H (2×25 cm) column as immobile phase and 20% MeOH (0.2% DEA)/CO₂, 100 bar, 60 mL/min. MS (ESI): mass calcd. for C₂₄H₃₀N₈O, 446.3. m/z found, 447.2 [M+H]⁺. ¹H NMR (500 MHz, Methanol-d4) δ 8.53 (dt, J=7.2, 1.0 Hz, 1H), 7.66 (dd, J=1.9, 0.9 Hz, 1H), 7.59 (s, 1H), 7.03 (s, 1H), 6.95 (s, 1H), 6.87 (dd, J=7.1, 1.9 Hz, 1H), 4.37 (d, J=11.5 Hz, 1H), 4.29 (d, J=11.5 Hz, 1H), 3.93 (ddd, J=6.5, 5.5, 3.7 Hz, 1H), 3.87 (s, 3H), 2.99 (dd, J=10.8, 5.5 Hz, 1H), 2.82 (dd, J=8.8, 6.8 Hz, 1H), 2.53 (s, 3H), 2.48 (dd, J=10.8, 3.7 Hz, 1H), 2.38 (d, J=0.6 Hz, 3H), 2.31 (s, 3H), 2.29-2.18 (m, 2H), 0.97 (d, J=6.8 Hz, 3H).

### Example 369: CIS—*S, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine

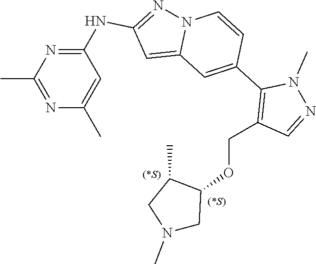

The title compound (5.4 mg, 2.2%) was prepared as described in Example 364 where Rac-cis-(3S,4R)-1,4-dimethylpyrrolidin-3-ol. The enantiomers were separated via chiral SFC using AS-H (2×25 cm) column as immobile phase and 20% MeOH (0.2% DEA)/CO₂, 100 bar, 60 mL/min. MS (ESI): mass calcd. for C₂₄H₃₀N₈O, 446.3. m/z found, 447.2 [M+H]⁺. ¹H NMR (400 MHz, Methanol-d4) δ 8.43 (dt, J=7.1, 0.9 Hz, 1H), 7.56 (dd, J=1.9, 0.9 Hz, 1H), 7.49 (s, 1H), 6.94 (s, 1H), 6.85 (s, 1H), 6.77 (dd, J=7.1, 1.9 Hz, 1H), 4.28 (d, J=11.5 Hz, 1H), 4.19 (d, J=11.5 Hz, 1H), 3.83 (td, J=5.8, 3.6 Hz, 1H), 3.77 (s, 3H), 2.90 (dd, J=10.8, 5.5 Hz, 1H), 2.79-2.66 (m, 1H), 2.43 (s, 3H), 2.40 (dd, J=10.8, 3.7 Hz, 1H), 2.29 (d, J=0.6 Hz, 3H), 2.23 (s, 3H), 2.20-2.10 (m, 2H), 0.87 (d, J=6.6 Hz, 3H).

### Example 370: CIS-Rac-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

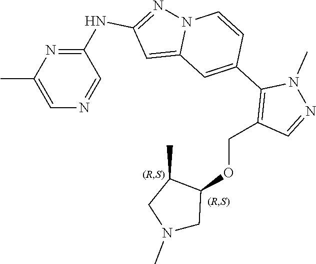

The title compound (4.7 mg, 2.0%) was prepared as described in Example 364. MS (ESI): mass calcd. for C₂₃H₂₈N₈O, 432.2. m/z found, 433.3 [M+H]⁺. ¹H NMR (400 MHz, Methanol-d4) δ 8.53 (dt, J=7.1, 0.9 Hz, 1H), 8.44 (s, 1H), 7.89 (s, 1H), 7.67 (dd, J=2.0, 1.0 Hz, 1H), 7.61 (s, 1H), 7.01 (d, J=0.8 Hz, 1H), 6.87 (dd, J=7.1, 1.9 Hz, 1H), 4.40 (d, J=11.5 Hz, 1H), 4.31 (d, J=11.5 Hz, 1H), 3.95 (td, J=5.8, 3.7 Hz, 1H), 3.89 (s, 3H), 3.02 (dd, J=10.8, 5.5 Hz, 1H), 2.85 (dd, J=8.5, 6.5 Hz, 1H), 2.55-2.45 (m, 4H), 2.34 (s, 3H), 2.31-2.20 (m, 2H), 0.99 (d, J=6.7 Hz, 3H).

### Example 371: Trans-*R, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

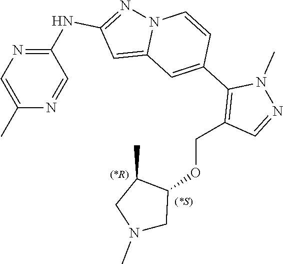

The title compound (10.4 mg, 40%) was prepared as described in Example 364. The enantiomers were separated via chiral SFC using AD-H (2×25 cm) column as immobile phase and 40% EtOH (0.1% DEA)/CO₂, 100 bar, 60 mL/min. MS (ESI): mass calcd. for C₂₃H₂₈N₈O, 432.2. m/z found, 433.3 [M+H]⁺. ¹H NMR (400 MHz, Methanol-d4) δ 8.56 (d, J=1.5 Hz, 1H), 8.50 (dt, J=7.2, 0.9 Hz, 1H), 8.17-8.09 (m, 1H), 7.62 (dd, J=1.9, 0.9 Hz, 1H), 7.59 (s, 1H), 6.89-6.79 (m, 2H), 4.37-4.25 (m, 2H), 3.87 (s, 3H), 3.68-3.57 (m, 1H), 2.96 (dd, J=9.5, 7.5 Hz, 1H), 2.76 (dd, J=10.7, 3.0 Hz, 1H), 2.58 (dd, J=10.7, 6.3 Hz, 1H), 2.44 (s, 3H), 2.30 (s, 3H), 2.21 (qd, J=7.3, 3.9 Hz, 1H), 2.00 (dd, J=9.5, 7.8 Hz, 1H), 1.04 (d, J=7.0 Hz, 3H).

### Example 372: Trans-*S, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

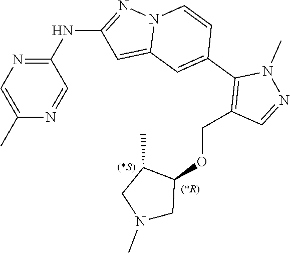

The title compound (11.1 mg, 43%) was prepared as described in Example 364. The enantiomers were separated via chiral SFC using AD-H (2×25 cm) column as immobile phase and 40% EtOH (0.1% DEA)/CO₂, 100 bar, 60 mL/min. MS (ESI): mass calcd. for C₂₃H₂₈N₈O, 432.2. m/z found, 433.3 [M+H]⁺. ¹H NMR (400 MHz, Methanol-d4) δ 8.56 (d, J=1.5 Hz, 1H), 8.50 (dt, J=7.2, 0.9 Hz, 1H), 8.17-8.09 (m, 1H), 7.62 (dd, J=1.9, 0.9 Hz, 1H), 7.59 (s, 1H), 6.89-6.79 (m, 2H), 4.37-4.25 (m, 2H), 3.87 (s, 3H), 3.68-3.57 (m, 1H), 2.96 (dd, J=9.5, 7.5 Hz, 1H), 2.76 (dd, J=10.7, 3.0 Hz, 1H), 2.58 (dd, J=10.7, 6.3 Hz, 1H), 2.44 (s, 3H), 2.30 (s, 3H), 2.21 (qd, J=7.3, 3.9 Hz, 1H), 2.00 (dd, J=9.5, 7.8 Hz, 1H), 1.04 (d, J=7.0 Hz, 3H).

### Example 373: (rac-trans)-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

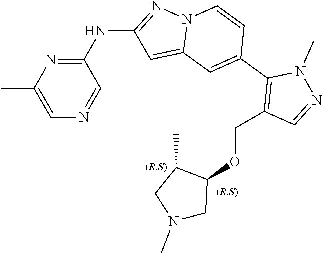

The title compound (6.4 mg, 1.9%) was prepared as described in Example 364. MS (ESI): mass calcd. for C₂₃H₂₈N₈O, 432.2. m/z found, 433.3 [M+H]⁺. ¹H NMR (400 MHz, Methanol-d4) δ 8.53 (dt, J=7.1, 0.9 Hz, 1H), 8.43 (s, 1H), 7.89 (s, 1H), 7.68 (dd, J=1.9, 0.9 Hz, 1H), 7.61 (s, 1H), 7.02 (d, J=0.9 Hz, 1H), 6.88 (dd, J=7.1, 1.9 Hz, 1H), 4.44-4.26 (m, 2H), 3.90 (s, 3H), 3.66 (dt, J=6.6, 3.5 Hz, 1H), 3.06-2.91 (m, 1H), 2.77 (dd, J=10.7, 3.1 Hz, 1H), 2.59 (dd, J=10.6, 6.3 Hz, 1H), 2.50 (t, J=0.7 Hz, 3H), 2.31 (s, 3H), 2.23 (qd, J=7.4, 4.0 Hz, 1H), 2.01 (dd, J=9.5, 7.8 Hz, 1H), 1.07 (d, J=7.1 Hz, 3H).

### Example 374: Trans-*S, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

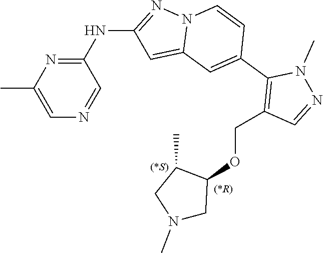

The title compound (82 mg, 24%) was prepared as described in Example 364. The enantiomers were separated via chiral SFC using AD-H (2×25 cm) column as immobile phase and 40% EtOH (0.1% DEA)/CO₂, 100 bar, 60 mL/min. MS (ESI): mass calcd. for C₂₃H₂₈N₈O, 432.2. m/z found, 433.3 [M+H]⁺. ¹H NMR (400 MHz, Methanol-d4) δ 8.53 (dt, J=7.1, 0.9 Hz, 1H), 8.43 (s, 1H), 7.89 (s, 1H), 7.68 (dd, J=1.9, 0.9 Hz, 1H), 7.61 (s, 1H), 7.02 (d, J=0.8 Hz, 1H), 6.88 (dd, J=7.1, 1.9 Hz, 1H), 4.43-4.26 (m, 2H), 3.90 (s, 3H), 3.66 (dt, J=6.5, 3.2 Hz, 1H), 3.02-2.93 (m, 1H), 2.77 (dd, J=10.7, 3.1 Hz, 1H), 2.59 (dd, J=10.7, 6.3 Hz, 1H), 2.50 (t, J=0.7 Hz, 3H), 2.31 (s, 3H), 2.23 (qd, J=7.2, 3.9 Hz, 1H), 2.01 (dd, J=9.5, 7.7 Hz, 1H), 1.07 (d, J=7.1 Hz, 3H).

### Example 375: Trans-*R, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

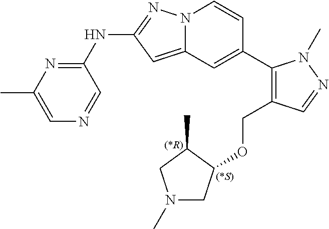 [SMILES: Cc1cncc(Nc2cc3cc(-c4c(CO[C@@H]5CN(C)C[C@H]5C)cnn4C)ccn3n2)n1]

The title compound (80 mg, 24%) was prepared as described in Example 364. The enantiomers were separated via chiral SFC using AD-H (2×25 cm) column as immobile phase and 40% EtOH (0.1% DEA)/CO₂, 100 bar, 60 mL/min. MS (ESI): mass calcd. for C₂₃H₂₈N₈O, 432.2. m/z found, 433.2 [M+H]⁺. ¹H NMR (400 MHz, Methanol-d4) δ 8.53 (dt, J=7.1, 0.9 Hz, 1H), 8.43 (s, 1H), 7.89 (s, 1H), 7.68 (dd, J=2.0, 0.9 Hz, 1H), 7.61 (s, 1H), 7.02 (d, J=0.9 Hz, 1H), 6.88 (dd, J=7.1, 1.9 Hz, 1H), 4.40-4.27 (m, 2H), 3.90 (s, 3H), 3.70-3.61 (m, 1H), 2.98 (dd, J=9.4, 7.5 Hz, 1H), 2.77 (dd, J=10.6, 3.0 Hz, 1H), 2.60 (dd, J=10.7, 6.3 Hz, 1H), 2.50 (d, J=0.7 Hz, 3H), 2.32 (s, 3H), 2.23 (qd, J=7.3, 3.9 Hz, 1H), 1.07 (d, J=7.1 Hz, 3H).

### Example 376: Rac-CIS-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

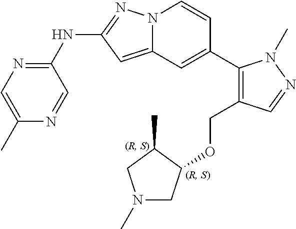 [SMILES: Cc1cnc(Nc2cc3cc(-c4c(CO[C@@H]5CN(C)C[C@H]5C)cnn4C)ccn3n2)cn1]

The title compound (4.3 mg, 1.8%) was prepared as described in Example 364. MS (ESI): mass calcd. for C₂₃H₂₈N₈O, 432.2. m/z found, 433.2 [M+H]⁺. ¹H NMR (400 MHz, Methanol-d4) δ 8.57 (d, J=1.5 Hz, 1H), 8.51 (dt, J=7.0, 0.9 Hz, 1H), 8.14 (dd, J=1.5, 0.8 Hz, 1H), 7.61 (dd, J=1.9, 0.9 Hz, 1H), 7.58 (s, 1H), 6.86-6.80 (m, 2H), 4.38 (d, J=11.5 Hz, 1H), 4.29 (d, J=11.5 Hz, 1H), 3.93 (td, J=5.9, 3.8 Hz, 1H), 3.87 (s, 3H), 2.99 (dd, J=10.8, 5.6 Hz, 1H), 2.82 (dd, J=8.6, 6.6 Hz, 1H), 2.48 (d, J=3.8 Hz, 1H), 2.45 (d, J=0.6 Hz, 4H), 2.35-2.18 (m, 6H), 0.97 (d, J=6.7 Hz, 3H).

### Example 377: CIS—*R, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

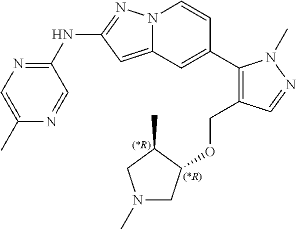 [SMILES: Cc1cnc(Nc2cc3cc(-c4c(CO[C@@H]5CN(C)C[C@H]5C)cnn4C)ccn3n2)cn1]

The title compound (48.8 mg, 20%) was prepared as described in Example 364 where Rac-cis-(3S,4R)-1,4-dimethylpyrrolidin-3-ol. The enantiomers were separated via chiral SFC using AS-H (2×25 cm) column as immobile phase and 18% EtOH (0.2% DEA)/CO₂, 100 bar, 60 mL/min. MS (ESI): mass calcd. for C₂₃H₂₈N₈O, 432.2. m/z found, 433.2 [M+H]⁺. ¹H NMR (400 MHz, Methanol-d4) δ 8.55 (d, J=1.5 Hz, 1H), 8.48 (dt, J=7.1, 0.9 Hz, 1H), 8.14-8.08 (m, 1H), 7.60-7.54 (m, 2H), 6.85-6.77 (m, 2H), 4.35 (d, J=11.5 Hz, 1H), 4.26 (d, J=11.5 Hz, 1H), 3.92 (td, J=5.7, 3.6 Hz, 1H), 3.85 (s, 3H), 2.99 (dd, J=10.8, 5.4 Hz, 1H), 2.88-2.76 (m, 1H), 2.49 (dd, J=10.8, 3.7 Hz, 1H), 2.43 (s, 3H), 2.32 (s, 3H), 2.30-2.20 (m, 2H), 0.96 (d, J=6.6 Hz, 3H).

### Example 378: CIS—*R, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

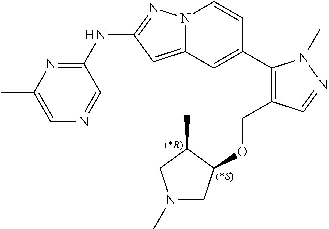 [SMILES: Cc1cncc(Nc2cc3cc(-c4c(CO[C@H]5CN(C)C[C@H]5C)cnn4C)ccn3n2)n1]

The title compound (48 mg, 20%) was prepared as described in Example 364. The enantiomers were separated via chiral SFC using AS-H (2×25 cm) column as immobile phase and 15-25% EtOH (0.2% DEA)/CO₂, 100 bar, 65 mL/min. MS (ESI): mass calcd. for C₂₃H₂₈N₈O, 432.2. m/z found, 433.2 [M+H]⁺. ¹H NMR (500 MHz, Methanol-d4) δ 8.49 (dd, J=7.1, 1.1 Hz, 1H), 8.41 (s, 1H), 7.86 (s, 1H), 7.63 (dd, J=1.9, 0.9 Hz, 1H), 7.58 (s, 1H), 6.97 (d, J=0.8 Hz, 1H), 6.83 (dd, J=7.1, 1.9 Hz, 1H), 4.36 (d, J=11.5 Hz, 1H), 4.27 (d, J=11.5 Hz, 1H), 3.92 (td, J=6.0, 3.7 Hz, 1H), 3.87 (s, 3H), 2.99 (dd, J=10.8, 5.5 Hz, 1H), 2.81 (dd, J=8.8, 6.8 Hz, 1H), 2.47 (s, 4H), 2.31 (s, 3H), 2.28-2.15 (m, 2H), 0.97 (d, J=6.8 Hz, 3H).

### Example 379: CIS—*S, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine

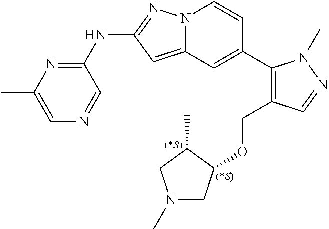 [SMILES: Cc1cncc(Nc2cc3cc(-c4c(CO[C@@H]5CN(C)C[C@@H]5C)cnn4C)ccn3n2)n1]

The title compound (51 mg, 21%) was prepared as described in Example 364. The enantiomers were separated via chiral SFC using AS-H (2×25 cm) column as immobile phase and 15-25% EtOH (0.2% DEA)/CO₂, 100 bar, 65 mL/min. MS (ESI): mass calcd. for C₂₃H₂₈N₈O, 432.2. m/z found, 433.3 [M+H]⁺. ¹H NMR (500 MHz, Methanol-d4) δ 8.39 (dt, J=7.2, 1.0 Hz, 1H), 8.30 (s, 1H), 7.76 (s, 1H), 7.52 (dd, J=1.9, 0.9 Hz, 1H), 7.47 (s, 1H), 6.87 (d, J=0.8 Hz, 1H), 6.72 (dd, J=7.1, 1.9 Hz, 1H), 4.26 (d, J=11.5 Hz, 1H), 4.17 (d, J=11.5 Hz, 1H), 3.82 (td, J=5.8, 3.6 Hz, 1H), 3.76 (s, 3H), 2.89 (dd, J=10.8, 5.5 Hz, 1H), 2.72 (dd, J=8.6, 6.6 Hz, 1H), 2.37 (s, 4H), 2.21 (s, 3H), 2.18-2.07 (m, 2H), 0.86 (d, J=6.7 Hz, 3H).

### Example 380: CIS—*S, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

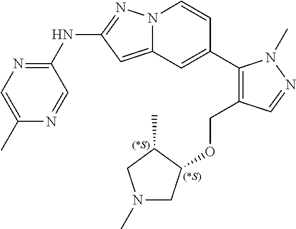 [SMILES: Cc1cnc(Nc2cc3cc(-c4c(CO[C@@H]5CN(C)C[C@@H]5C)cnn4C)ccn3n2)cn1]

The title compound (50 mg, 21%) was prepared as described in Example 364 where Rac-cis-(3S,4R)-1,4-dimethylpyrrolidin-3-ol. The enantiomers were separated via chiral SFC using AS-H (2×25 cm) column as immobile phase and 18% EtOH (0.2% DEA)/CO₂, 100 bar, 60 mL/minMS (ESI): mass calcd. for C₂₃H₂₈N₈O, 432.2. m/z found, 433.2 [M+H]⁺. ¹H NMR (400 MHz, Methanol-d4) δ 8.58 (d, J=1.5 Hz, 1H), 8.52 (dt, J=7.1, 1.0 Hz, 1H), 8.15 (dd, J=1.5, 0.7 Hz, 1H), 7.63 (dd, J=2.0, 0.9 Hz, 1H), 7.60 (s, 1H), 6.90-6.81 (m, 2H), 4.40 (d, J=11.5 Hz, 1H), 4.30 (d, J=11.5 Hz, 1H), 3.95 (td, J=5.8, 3.7 Hz, 1H), 3.89 (s, 3H), 3.02 (dd, J=10.8, 5.5 Hz, 1H), 2.92-2.80 (m, 1H), 2.52 (dd, J=10.8, 3.7 Hz, 1H), 2.46 (d, J=0.5 Hz, 3H), 2.35 (s, 3H), 2.34-2.22 (m, 2H), 0.99 (d, J=6.6 Hz, 3H).

### Example 381: N-(5-tert-butylpyrazin-2-yl)-5-[2-methyl-4-[[(2R)-1-methylazetidin-2-yl]methoxy]pyrazol-3-yl]pyrazolo[1,5-a]pyridin-2-amine

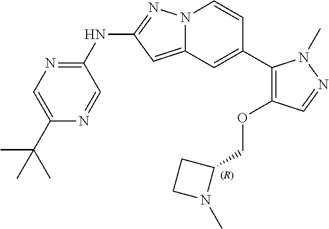

The title compound (40 mg, 3 steps, 7%) was prepared as described in Example 2, except 2-tert-butyl-5-chloropyrazine was used instead of 4-chloro-2,6-dimethylpyrimidine in Step A. MS (ESI): mass calcd. for C₂₄H₃₀N₈O, 446.3. m/z found, 447.25 [M+H]⁺. ¹H NMR (400 MHz, Chloroform-d) δ 8.69 (d, J=1.5 Hz, 1H), 8.36 (dt, J=7.2, 0.9 Hz, 1H), 8.28 (d, J=1.5 Hz, 1H), 7.68 (s, 1H), 7.46 (dd, J=1.9, 0.9 Hz, 1H), 7.36 (s, 1H), 6.83 (dd, J=7.2, 1.9 Hz, 1H), 6.69 (d, J=0.8 Hz, 1H), 3.99 (dd, J=5.5, 1.4 Hz, 2H), 3.92 (s, 3H), 3.53-3.39 (m, 1H), 3.38-3.29 (m, 1H), 2.87-2.80 (m, 1H), 2.35 (s, 3H), 2.09-1.92 (m, 2H), 1.40 (s, 9H).

### Example 382: N-[5-[5-methyl-3-[(3S)-1-methylpyrrolidin-3-yl]oxy-isoxazol-4-yl]pyrazolo[1,5-a]pyridin-2-yl]cyclopropanecarboxamide

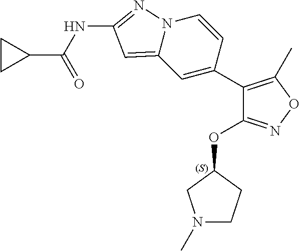

Step A. (S)—N-(5-(5-methyl-3-(pyrrolidin-3-yloxy)isoxazol-4-yl)pyrazolo[1,5-a]pyridin-2-yl)cyclopropanecarboxamide. The title compound was prepared as described in Steps A-D of Example 32, except (R)-tert-butyl 3-(tosyloxy)pyrrolidine-1-carboxylate was used instead of (S)-2-((tert-butoxycarbonyl)amino)-2-phenylethyl 4-methylbenzenesulfonate in Step A.

Step B. N-[5-[5-methyl-3-[(3S)-1-methylpyrrolidin-3-yl]oxy-isoxazol-4-yl]pyrazolo[1,5-a]pyridin-2-yl]cyclopropanecarboxamide. The title compound (40 mg, 28%) was prepared as described in Step A of Example 24, except (S)—N-(5-(5-methyl-3-(pyrrolidin-3-yloxy)isoxazol-4-yl)pyrazolo[1,5-a]pyridin-2-yl)cyclopropanecarboxamide was used instead of (R)-5-(4-(azetidin-2-ylmethoxy)-1-methyl-1H-pyrazol-5-yl)-N-(5,6-dimethylpyrazin-2-yl)pyrazolo[1,5-a]pyridin-2-amine. MS (ESI): mass calcd. for C₂₀H₂₃N₅O₃, 381.2. m/z found, 382.2 [M+H]⁺. ¹H NMR (500 MHz, DMSO-d6) δ 11.04 (s, 1H), 8.56 (d, J=7.2 Hz, 1H), 7.62 (dd, J=2.0, 0.9 Hz, 1H), 6.88 (dd, J=7.2, 2.0 Hz, 1H), 6.85 (s, 1H), 5.19-5.06 (m, 1H), 2.78-2.68 (m, 3H), 2.51 (s, 3H), 2.32-2.27 (m, 2H), 2.26-2.23 (m, 3H), 1.95-1.85 (m, 2H), 0.91-0.70 (m, 4H).

### Example 383: N-[5-[5-methyl-3-[[(3R)-3-piperidyl]methoxy]isoxazol-4-yl]pyrazolo[1,5-a]pyridin-2-yl]cyclopropanecarboxamide

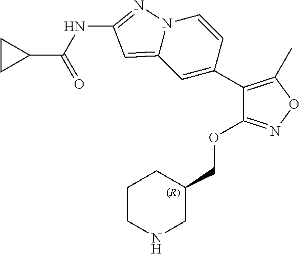

Step A. Tert-butyl (R)-3-((tosyloxy)methyl)piperidine-1-carboxylate. To a solution of (R)-1-boc-3-(hydroxymethyl)piperidine (2000 mg, 9.29 mmol), DMAP (1248.1 mg, 10.219 mmol), in DCM (11.9 mL, 185.79 mmol) was added p-toluenesulfonyl chloride (1771 mg, 9.29 mmol) in one portion at 23° C. After 2 hr to the reaction mixture was added silica gel and the mixture was concentrated in vacuo to give a clear colorless oil (3080 mg, 89.7%)

Step B. N-[5-[5-methyl-3-[[(3R)-3-piperidyl]methoxy]isoxazol-4-yl]pyrazolo[1,5-a]pyridin-2-yl]cyclopropanecarboxamide. The title compound (115 mg, 3 steps, 25%) was prepared as described in Steps A-C of Example 32, except tert-butyl (R)-3-((tosyloxy)methyl)piperidine-1-carboxylate was used instead of (S)-2-((tert-butoxycarbonyl)amino)-2-phenylethyl 4-methylbenzenesulfonate in Step A. MS (ESI): mass calcd. for C₂₁H₂₅N₅O₃, 395.2. m/z found, 396.1 [M+H]⁺. ¹H NMR (400 MHz, DMSO-d6) δ 11.05 (s, 1H), 8.58 (d, J=7.1 Hz, 1H), 7.62 (s, 1H), 6.88 (dd, J=7.2, 2.0 Hz, 1H), 6.84 (s, 1H), 4.12 (dd, J=6.6, 1.8 Hz, 2H), 2.98 (d, J=12.0 Hz, 1H), 2.81 (d, J=12.1 Hz, 1H), 2.51 (s, 3H), 2.46-2.37 (m, 1H), 2.37-2.27 (m, 1H), 1.99-1.85 (m, 2H), 1.76 (d, J=12.7 Hz, 1H), 1.56 (d, J=12.9 Hz, 1H), 1.41-1.28 (m, 1H), 1.26-1.11 (m, 1H), 0.87-0.70 (m, 4H).

### Example 384: N-[5-[5-methyl-3-[(3S)-1-(trideuteriomethyl)pyrrolidin-3-yl]oxy-isoxazol-4-yl]pyrazolo[1,5-a]pyridin-2-yl]cyclopropanecarboxamide

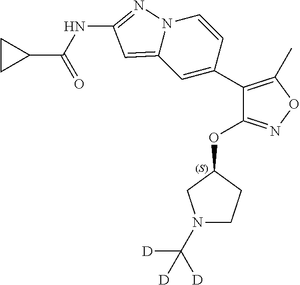

The title compound (20 mg, 5 steps, 9%) was prepared as described in Example 1 Steps B-F, except in Step B (R)-tert-butyl 3-(tosyloxy)pyrrolidine-1-carboxylate was used instead of (2S,4R)-2-methyl-4-(tosyloxy)pyrrolidine-1-carboxylate and 4-bromo-5-methylisoxazol-3-ol was used instead of 1-methyl-1h-pyrazol-4-ol. In Step F paraformaldehyde-D2 (1.5 eqiuv.) and sodium cyanoborodeutride were used instead of paraformaldehyde and sodium cyanoborohydride, respectively. MS (ESI): mass calcd. for C₂₀H₂₃N₅O₃, 384.2. m/z found, 385.2 [M+H]⁺. ¹H NMR (500 MHz, DMSO-d6) δ 11.04 (s, 1H), 8.55 (d, J=7.3 Hz, 1H), 7.62 (dd, J=2.0, 1.0 Hz, 1H), 6.88 (dd, J=7.2, 2.0 Hz, 1H), 6.86-6.82 (m, 1H), 5.17-5.06 (m, 1H), 2.75 (dd, J=11.0, 2.6 Hz, 1H), 2.72-2.64 (m, 2H), 2.51 (s, 3H), 2.34-2.23 (m, 2H), 1.97-1.82 (m, 2H), 0.88-0.74 (m, 4H).

### Example 385: (3*R,4*S)—N-[5-[3-(4-methoxypyrrolidin-3-yl)oxy-5-methyl-isoxazol-4-yl]pyrazolo[1,5-a]pyridin-2-yl]cyclopropanecarboxamide

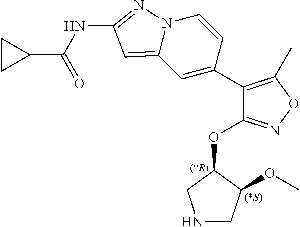

The title compound (17.6 mg, 3 steps, 1.5%) was prepared as described in Example 32, except tert-butyl (3R,4R)-3-methoxy-4-((methylsulfonyl)oxy)pyrrolidine-1-carboxylate was used instead of (S)-2-((tert-butoxycarbonyl)amino)-2-phenylethyl 4-methylbenzenesulfonate in Step A. MS (ESI): mass calcd. for C₂₀H₂₃N₅O₄, 397.2. m/z found, 398.2 [M+H]⁺. ¹H NMR (500 MHz, DMSO-d6) δ 11.04 (s, 1H), 8.60-8.51 (m, 1H), 7.72-7.64 (m, 1H), 6.96 (dd, J=7.2, 2.0 Hz, 1H), 6.83 (s, 1H), 5.09-5.01 (m, 1H), 3.96-3.87 (m, 1H), 3.27 (s, 3H), 3.24 (dd, J=12.3, 5.6 Hz, 1H), 3.08 (dd, J=11.1, 6.4 Hz, 1H), 2.89 (dd, J=12.4, 4.1 Hz, 1H), 2.73 (dd, J=11.2, 6.7 Hz, 1H), 2.53 (s, 3H), 1.98-1.86 (m, 1H), 0.90-0.70 (m, 4H).

### Example 386: N-[5-[3-(3-aminocyclobutoxy)-5-methyl-isoxazol-4-yl]pyrazolo[1,5-a]pyridin-2-yl]cyclopropanecarboxamide

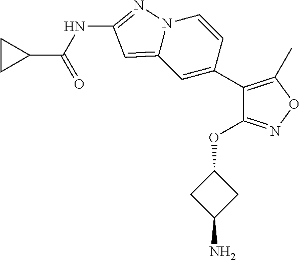 [SMILES: Cc1onc(O[C@H]2C[C@H](N)C2)c1-c1ccn2nc(NC(=O)C3CC3)cc2c1]

Step A. (1s,3s)-3-((tert-butoxycarbonyl)amino)cyclobutyl 4-methylbenzenesulfonate. To dichloromethane (2 mL) was dissolved tert-butyl-3-hydroxycyclobutylcarbamate (50.0 mg, 0.267 mmol), p-toluenesulfonyl chloride (56.0 mg, 0.294 mmol) and DMAP (65.2 mg, 0.534 mmol). The reaction mixture was left to stir overnight. The organic phase was washed with 1N HCl (2 mL×2), saturated aqueous NaHCO₃ (2 mL×2) and concentrated under reduced pressure to afford the title compound (70 mg, 77%) as a white solid.

Step B. Tert-butyl ((1r,3r)-3-((5-methylisoxazol-3-yl)oxy)cyclobutyl)carbamate. A solution consisting of (cis)-3-((tert-butoxycarbonyl)amino)cyclobutyl 4-methylbenzenesulfonate (207 mg, 0.606 mmol), 3-hydroxy-5-methylisoxazole (60.0 mg, 0.606 mmol), K₂CO₃ (592 mg, 1.82 mmol) and CH₃CN (10 mL) was stirred at 80° C. The mixture was diluted with water (10 mL) and the solution was extracted with ethyl acetate (10 mL×2), then the combined organic layers were concentrated under reduced pressure to give the crude product as a yellow oil (200 mg, 78%) which was used to the next step without further purification.

Step C. Tert-butyl ((1r,3r)-3-((4-(2-(cyclopropanecarboxamido)pyrazolo[1,5-a]pyridin-5-yl)-5-methylisoxazol-3-yl)oxy)cyclobutyl)carbamate. A solution consisting of N-(5-bromopyrazolo[1,5-a]pyridin-2-yl)cyclopropanecarboxamide (122 mg, 0.436 mmol), 4-bromo-1-methyl-1H-1,2,3-triazole (150 mg, 0.363 mmol), Pd(OAc)₂ (12 mg, 0.055 mmol), KOAc (71.3 mg, 0.727 mmol) and DMA (3 mL) was stirred at 130° C. under microwave irradiation for 60 minutes. The reaction mixture was diluted with water (30 mL) and the mixture was extracted with ethyl acetate (40 mL×2), then the combined organic layers were concentrated under reduced pressure to give the crude product which was purified by FCC (petroleum ether:ethyl acetate=1:0 to 1:1) to afford the desired product as a yellow oil (200 mg, crude).

Step D. N-[5-[3-(3-aminocyclobutoxy)-5-methyl-isoxazol-4-yl]pyrazolo[1,5-a]pyridin-2-yl]cyclopropanecarboxamide. To a solution consisting of tert-butyl ((trans)-3-((4-(2-(cyclopropanecarboxamido)pyrazolo[1,5-a]pyridin-5-yl)-5-methylisoxazol-3-yl)oxy)cyclobutyl)carbamate (200 mg, crude) under 0° C. was added TFA (0.284 mL, 3.84 mmol) and the solution was stirred for 1 h, then the solution was warmed to room-temperature and stirred for 1 h. The reaction mixture was concentrated under reduced pressure to give the crude product, and the saturated NaHCO₃ aqueous solution until pH=7-8, the crude solution was dried under high vacuum to afford the title compound as a brown solid (200 mg, crude). The crude product was purified by preparative HPLC with Boston Prime C18 150×30 mm×5 um, column (eluent: 20% to 50% (v/v) CH₃CN and H₂O with 0.04% NH₃H₂O+10 mM NH₄HCO₃) to afford pure product. The product was suspended in water (10 mL), the mixture frozen using dry ice/EtOH, and then lyophilized to dryness to afford the title compound as a white solid (11.7 mg, 5.3%). MS (ESI): mass calcd. for C₁₉H₂₁N₅O₃, 367.2. m/z found, 368.2 [M+H]⁺. ¹H NMR (400 MHz, DMSO-d6) δ 11.06 (s, 1H), 8.57 (d, J=7.2 Hz, 1H), 7.64 (s, 1H), 6.90 (dd, J=1.8, 7.2 Hz, 1H), 6.86 (s, 1H), 5.18-5.06 (m, 1H), 3.66-3.54 (m, 1H), 2.50-2.48 (m, 3H), 2.40-2.29 (m, 2H), 2.22-2.07 (m, 2H), 2.00-1.85 (m, 1H), 0.96-0.77 (m, 4H)

### Example 387: N-[5-[3-[(3-aminocyclobutyl)methoxy]-5-methyl-isoxazol-4-yl]pyrazolo[1,5-a]pyridin-2-yl]cyclopropanecarboxamide

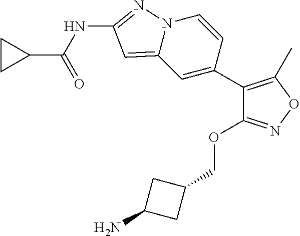 [SMILES: Cc1onc(OC[C@H]2C[C@H](N)C2)c1-c1ccn2nc(NC(=O)C3CC3)cc2c1]

The title compound (107.8 mg, 4 steps, 18.8%) was prepared as described in Example 386, except tert-butyl ((trans)-3-(hydroxymethyl)cyclobutyl)carbamate was used instead of tert-butyl-3-hydroxycyclobutylcarbamate in Step A. MS (ESI): mass calcd. for C₂₀H₂₃N₅O₃, 381.2. m/z found, 382.2 [M+H]⁺. ¹H NMR (400 MHz, CD3OD): 8.39 (d, J=7.6 Hz, 1H), 7.57 (s, 1H), 6.91 (dd, J=2.0, 7.2 Hz, 1H), 6.84 (s, 1H), 4.34 (d, J=6.4 Hz, 2H), 3.65-3.56 (m, 1H), 2.81-2.67 (m, 1H), 2.52 (s, 3H), 2.32-2.22 (m, 2H), 2.12-2.01 (m, 2H), 1.91-1.82 (m, 1H), 1.03-0.96 (m, 2H), 0.94-0.86 (m, 2H)

### Example 388: N-[5-[5-methyl-3-[[(2R)-1-methylpyrrolidin-2-yl]methoxy]isoxazol-4-yl]pyrazolo[1,5-a]pyridin-2-yl]cyclopropanecarboxamide

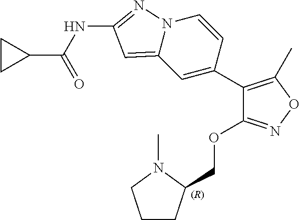

Step A. tert-butyl (R)-2-(((4-bromo-5-methylisoxazol-3-yl)oxy)methyl)pyrrolidine-1-carboxylate. The title compound (380 mg, 49%) was prepared as described in Example 386 (Steps A-B), except (R)-tert-butyl 2-(hydroxymethyl)pyrrolidine-1-carboxylate was used instead of tert-butyl-3-hydroxycyclobutylcarbamate in Step A. In Step B cesium carbonate was used instead of potassium carbonate and DMF (8 mL) was used instead of acetonitrile.

Step B. N-[5-[5-methyl-3-[[(2R)-1-methylpyrrolidin-2-yl]methoxy]isoxazol-4-yl]pyrazolo[1,5-a]pyridin-2-yl]cyclopropanecarboxamide. The title compound (41.4 mg, 3%) was prepared as described in Example 5, except tert-butyl (R)-2-(((4-bromo-5-methylisoxazol-3-yl)oxy)methyl)pyrrolidine-1-carboxylate was used instead of rac-cis-tert-butyl (3s,4r)-3-((5-bromo-1-methyl-1H-pyrazol-4-yl)oxy)-4-methoxypyrrolidine-1-carboxylate in Step D.

Step C. N-[5-[5-methyl-3-[[(2R)-1-methylpyrrolidin-2-yl]methoxy]isoxazol-4-yl]pyrazolo[1,5-a]pyridin-2-yl]cyclopropanecarboxamide. The title compound (39.7 mg, 1%) was prepared as described in Example 24, except N-[5-[5-methyl-3-[[(2R)-1-methylpyrrolidin-2-yl]methoxy]isoxazol-4-yl]pyrazolo[1,5-a]pyridin-2-yl]cyclopropanecarboxamide was used instead of (R)-5-(4-(azetidin-2-ylmethoxy)-1-methyl-1H-pyrazol-5-yl)-N-(5,6-dimethylpyrazin-2-yl)pyrazolo[1,5-a]pyridin-2-amine. MS (ESI): mass calcd. for C₂₁H₂₅N₅O₃, 395.2. m/z found, 396.1 [M+H]⁺. ¹H NMR (400 MHz, CD3OD): 8.39 (d, J=7.2 Hz, 1H), 7.61 (d, J=0.8 Hz, 1H), 6.93 (dd, J=2.0, 7.2 Hz, 1H), 6.83 (s, 1H), 4.37-4.23 (m, 2H), 3.10-3.02 (m, 1H), 2.85-2.75 (m, 1H), 2.52 (s, 3H), 2.43 (s, 3H), 2.39-2.27 (m, 1H), 2.14-2.02 (m, 1H), 1.92-1.76 (m, 3H), 1.75-1.63 (m, 1H), 1.02-0.95 (m, 2H), 0.94-0.86 (m, 2H)

### Example 389: N-[5-[3-[(3S,5S)-5-(difluoromethyl)pyrrolidin-3-yl]oxy-5-methyl-isoxazol-4-yl]pyrazolo[1,5-a]pyridin-2-yl]cyclopropanecarboxamide

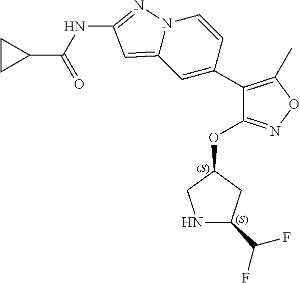

Step A. Tert-butyl (2S,4S)-4-((4-bromo-5-methylisoxazol-3-yl)oxy)-2 (difluoromethyl)pyrrolidine-1-carboxylate. To a round bottom flask containing a stir-bar was added tert-butyl (2S,4RS)-2-(difluoromethyl)-4-hydroxypyrrolidine-1-carboxylate (1 g, 4.215 mmol), di-tert-butyl azodicarboxylate (1.26 g, 5.48 mmol), 4-bromo-5-methylisoxazol-3-ol (750 mg, 4.22 mmol) and then THF (15.44 mL) by syringe under N₂. To the solution was then added TPP (1.44 g, 5.48 mmol) in one portion at 23° C.

Step B. N-[5-[3-[(3S,5S)-5-(difluoromethyl)pyrrolidin-3-yl]oxy-5-methyl-isoxazol-4-yl]pyrazolo[1,5-a]pyridin-2-yl]cyclopropanecarboxamide. The title compound (74 mg, 2 steps, 36%) was prepared as described in steps D-E of Example 5, except tert-butyl (2S,4S)-4-((4-bromo-5-methylisoxazol-3-yl)oxy)-2 (difluoromethyl)pyrrolidine-1-carboxylate was used instead of rac-cis-tert-butyl (3s,4r)-3-((5-bromo-1-methyl-1H-pyrazol-4-yl)oxy)-4-methoxypyrrolidine-1-carboxylate in step D. MS (ESI): mass calcd. for C₂₀H₂₁F₂N₅O₃, 417.2. m/z found, 418.2 [M+H]⁺. ¹H NMR (400 MHz, DMSO-d6) δ 11.05 (s, 1H), 8.56 (dd, J=7.2, 1.0 Hz, 1H), 7.63 (dd, J=2.0, 0.9 Hz, 1H), 6.89 (dd, J=7.2, 2.0 Hz, 1H), 6.84 (s, 1H), 5.76 (td, J=56.9, 5.5 Hz, 1H), 5.22-5.10 (m, 1H), 3.51-3.37 (m, 1H), 3.22 (dd, J=11.7, 5.3 Hz, 1H), 3.02 (dd, J=11.7, 3.0 Hz, 1H), 2.52 (s, 3H), 2.44-2.33 (m, 1H), 1.99-1.86 (m, 2H), 0.88-0.73 (m, 4H).

### Example 390: N-(5,6-dimethylpyridazin-3-yl)-5-[3-[(3S,5S)-5-(methoxymethyl)pyrrolidin-3-yl]oxy-5-methyl-isoxazol-4-yl]pyrazolo[1,5-a]pyridin-2-amine

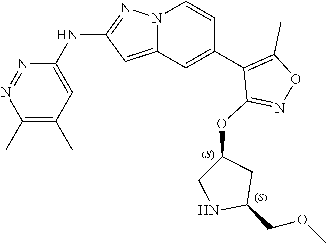

Step A. Tert-butyl (2S,4R)-4-hydroxy-2-(methoxymethyl)pyrrolidine-1-carboxylate. A solution consisting of (3R,5S)-5-(methoxymethyl)pyrrolidin-3-ol hydrochloride (900 mg, 5.37 mmol), Boc₂O (1.17 g, 5.37 mmol), TEA (1.50 mL, 10.7 mmol), and MeOH (10 mL) was stirred overnight at room-temperature. The reaction mixture was concentrated to dryness under reduced pressure. The residue was partitioned between water (30 mL) and ethyl acetate (50 mL). The organic extract was separated, dried over anhydrous Na₂SO₄, filtered, and evaporated to dryness under reduced pressure to give the title compound as a light yellow oil (1.2 g, 97%).

Step B. Tert-butyl (2S,4S)-4-((4-bromo-5-methylisoxazol-3-yl)oxy)-2-(methoxymethyl)pyrrolidine-1-carboxylate. To a solution consisting of 4-bromo-5-methylisoxazol-3-ol (450 mg, 2.52 mmol), (2S,4R)-tert-butyl 4-hydroxy-2-(methoxymethyl)pyrrolidine-1-carboxylate (643 mg, 2.78 Mmol), and PPh₃ (995 mg, 3.79 mmol), and dry toluene (10 mL) under N₂ was added DIAD (1.02 g, 5.06 mmol) dropwise. The resultant mixture was stirred for 3 h at 110° C. The mixture was concentrated under reduced pressure to give a crude product which was purified by FCC (eluent: petroleum ether:CH2Cl2=1:0 to 3:1) to afford P1 (900 mg, 91%) as a yellow oil.

Step C. Tert-butyl (2S,4S)-4-((4-(2-aminopyrazolo[1,5-a]pyridin-5-yl)-5-methylisoxazol-3-yl)oxy)-2-(methoxymethyl)pyrrolidine-1-carboxylate. The title compound was prepared as described in Step D of Example 1, except tert-butyl (2S,4S)-4-((4-bromo-5-methylisoxazol-3-yl)oxy)-2-(methoxymethyl)pyrrolidine-1-carboxylate was used instead of tert-butyl (2S,4S)-4-((5-bromo-1-methyl-1H-pyrazol-4-yl)oxy)-2-methylpyrrolidine-1-carboxylate and 5-(4,4,5,5-tetramethyl-1,3,2-dioxaborolan-2-yl)pyrazolo[1,5-a]pyridin-2-amine was used instead of Intermediate 3.

Step D. Tert-butyl (2S,4S)-4-((4-(2-((5,6-dimethylpyridazin-3-yl)amino)pyrazolo[1,5-a]pyridin-5-yl)-5-methylisoxazol-3-yl)oxy)-2-(methoxymethyl)pyrrolidine-1-carboxylate. The title compound (23.8 mg, 2 steps, 12%) was prepared as described in Steps A-B of Example 17, except 6-chloro-3,4-dimethylpyridazine was used instead of Intermediate 6 (Step A) and tert-butyl (2S,4S)-4-((4-(2-((5,6-dimethylpyridazin-3-yl)amino)pyrazolo[1,5-a]pyridin-5-yl)-5-methylisoxazol-3-yl)oxy)-2-(methoxymethyl)pyrrolidine-1-carboxylate was used instead of tert-butyl (R)-2-(((5-(2-((5,6-dimethylpyridazin-3-yl)amino)pyrazolo[1,5-a]pyridin-5-yl)-1-methyl-1H-pyrazol-4-yl)oxy)methyl)azetidine-1-carboxylate in Step B. MS (ESI): mass calcd. for C₂₃H₂₇N₇O₃, 449.2. m/z found, 450.2 [M+H]⁺. ¹H NMR (400 MHz, DMSO-d6) δ 9.88 (s, 1H), 8.55 (d, J=7.2 Hz, 1H), 7.63 (d, J=0.8 Hz, 1H), 7.40 (s, 1H), 6.88-6.80 (m, 2H), 5.16-5.08 (m, 1H), 3.30-3.20 (m, 5H), 3.19-2.97 (m, 3H), 2.53 (s, 3H), 2.46 (s, 3H), 2.32-2.18 (m, 4H), 1.72-1.62 (m, 1H)

### Example 391: N-(5,6-dimethylpyridazin-3-yl)-5-[3-[(3S,5S)-5-(methoxymethyl)-1-methyl-pyrrolidin-3-yl]oxy-5-methyl-isoxazol-4-yl]pyrazolo[1,5-a]pyridin-2-amine

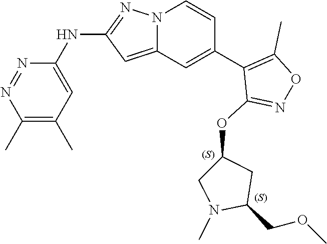

The title compound (27.2 mg, 4.2%) was prepared as described in Step B of Example 8, except N-(5,6-dimethylpyridazin-3-yl)-5-[3-[(3S,5S)-5-(methoxymethyl)pyrrolidin-3-yl]oxy-5-methyl-isoxazol-4-yl]pyrazolo[1,5-a]pyridin-2-amine (Example 390) was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₄H₂₉N₇O₃, 463.2. m/z found, 464.2 [M+H]⁺. ¹H NMR (400 MHz, CDCl₃) δ 8.33 (d, J=7.2 Hz, 1H), 8.20 (s, 1H), 7.70 (s, 1H), 7.43 (s, 1H), 6.79 (dd, J=1.6, 7.2 Hz, 1H), 6.53 (s, 1H), 5.19-5.13 (m, 1H), 3.53-3.47 (m, 1H), 3.47-3.40 (m, 2H), 3.36 (s, 3H), 2.62-2.53 (m, 5H), 2.51 (s, 3H), 2.48-2.42 (m, 1H), 2.39 (s, 3H), 2.32 (s, 3H), 2.02-1.93 (m, 1H)

### Example 392: 5-[4-[[(2R)-azetidin-2-yl]methoxy]-2-methyl-pyrazol-3-yl]-N-(6-methylpyridazin-3-yl)pyrazolo[1,5-a]pyridin-2-amine

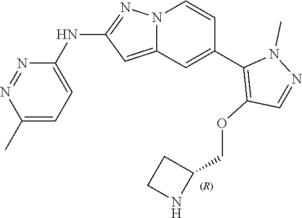

The title compound (78 mg, 2 steps, 32%) was prepared as described in Step A of Example 2 then Step B Example 2, except 3-bromo-6-methylpyridazine was used instead of 4-chloro-2,6-dimethylpyrimidine. In Step B Example 2, tert-butyl (R)-2-(((1-methyl-5-(2-((6-methylpyridazin-3-yl)amino)pyrazolo[1,5-a]pyridin-5-yl)-1H-pyrazol-4-yl)oxy)methyl)azetidine-1-carboxylate was used instead of tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. MS (ESI): mass calcd. for C₂₀H₂₂N₈O, 390.2. m/z found, 391.2 [M+H]⁺. ¹H NMR (500 MHz, Chloroform-d) δ 9.69 (s, 1H), 8.32 (d, J=7.0 Hz, 1H), 7.92 (d, J=9.2 Hz, 1H), 7.35 (dd, J=1.9, 0.9 Hz, 1H), 7.27 (s, 1H), 7.17 (d, J=9.1 Hz, 1H), 6.70 (dd, J=7.2, 1.9 Hz, 1H), 6.63 (s, 1H), 4.14 (s, 1H), 4.00-3.88 (m, 2H), 3.83 (d, J=6.7 Hz, OH), 3.82 (s, 3H), 3.57 (q, J=8.4, 7.9 Hz, 1H), 3.39 (d, J=6.4 Hz, OH), 3.37 (s, 1H), 2.85 (s, 2H), 2.55 (s, 2H), 2.32-2.22 (m, 1H), 2.17-2.06 (m, 1H).

### Example 393: 5-[4-[[(2R)-azetidin-2-yl]methoxy]-2-methyl-pyrazol-3-yl]-N-(5-methylpyrazin-2-yl)pyrazolo[1,5-a]pyridin-2-amine

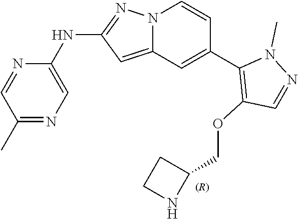

The title compound (91 mg, 2 steps, 37%) was prepared as described in Step A of Example 2, except 2-chloro-5-methylpyrazine was used instead of 4-chloro-2,6-dimethylpyrimidine. In Step B Example 2, tert-butyl (R)-2-(((1-methyl-5-(2-((5-methylpyrazin-2-yl)amino)pyrazolo[1,5-a]pyridin-5-yl)-1H-pyrazol-4-yl)oxy)methyl)azetidine-1-carboxylate was used instead of tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. MS (ESI): mass calcd. for C₂₀H₂₂N₈O, 390.2. m/z found, 391.2 [M+H]⁺. ¹H NMR (500 MHz, Chloroform-d) δ 8.54 (d, J=1.5 Hz, 1H), 8.30 (dt, J=7.1, 0.9 Hz, 1H), 8.03-7.98 (m, 1H), 7.37 (dd, J=2.0, 1.0 Hz, 1H), 7.28 (s, 1H), 6.73 (dd, J=7.2, 1.9 Hz, 1H), 6.62 (d, J=0.9 Hz, 1H), 4.20-4.11 (m, 1H), 4.00-3.91 (m, 2H), 3.82 (s, 3H), 3.59 (q, J=8.0 Hz, 1H), 3.43-3.35 (m, 1H), 2.88 (s, 2H), 2.41 (s, 3H), 2.31-2.24 (m, 1H), 2.18-2.09 (m, 1H).

### Example 394: 5-[4-[[(2R)-azetidin-2-yl]methoxy]-2-methyl-pyrazol-3-yl]-N-(6-cyclopropylpyridazin-3-yl)pyrazolo[1,5-a]pyridin-2-amine

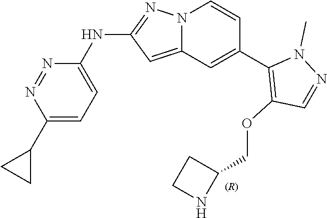

The title compound (113 mg, 2 steps, 22%) was prepared as described in Step A of Example 2, except 3-chloro-6-cyclopropylpyridazine was used instead of 4-chloro-2,6-dimethylpyrimidine. In Step B Example 2, tert-butyl (R)-2-(((5-(2-((6-cyclopropylpyridazin-3-yl)amino)pyrazolo[1,5-a]pyridin-5-yl)-1-methyl-1H-pyrazol-4-yl)oxy)methyl)azetidine-1-carboxylate was used instead of tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. MS (ESI): mass calcd. for C₂₂H₂₄N₈O, 416.2. m/z found, 417.20 [M+H]⁺. ¹H NMR (500 MHz, Chloroform-d) δ 9.38 (s, 1H), 8.30 (dd, J=7.1, 1.0 Hz, 1H), 7.85 (d, J=9.2 Hz, 1H), 7.31-7.23 (m, 2H), 7.08 (d, J=9.3 Hz, 1H), 6.69 (dd, J=7.1, 2.0 Hz, 1H), 6.59 (s, 1H), 4.27-4.21 (m, 1H), 4.02 (dd, J=10.0, 6.7 Hz, 1H), 3.96 (dd, J=10.0, 4.3 Hz, 1H), 3.81 (s, 3H), 3.64 (q, J=8.2 Hz, 1H), 3.52-3.38 (m, 2H), 2.35-3.27 (m, 1H), 2.20-2.12 (m, 1H), 2.05-1.99 (m, 1H), 1.01-0.96 (m, 4H).

### Example 395: N-(5-fluoropyrimidin-2-yl)-5-[2-methyl-4-[[(2R)-1-methylazetidin-2-yl]methoxy]pyrazol-3-yl]pyrazolo[1,5-a]pyridin-2-amine

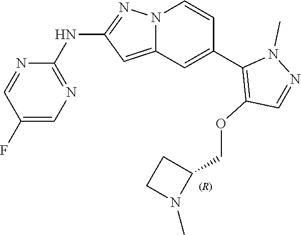

Step A. tert-butyl (R)-2-(((5-(2-((5-fluoropyrimidin-2-yl)amino)pyrazolo[1,5-a]pyridin-5-yl)-1-methyl-1H-pyrazol-4-yl)oxy)methyl)azetidine-1-carboxylate. The title compound (550 mg, 74%) was prepared as described in Step A of Example 36, except 2-chloro-5-fluoropyrimidine was used instead of methyl 6-bromonicotinate.

Step B. N-(5-fluoropyrimidin-2-yl)-5-[2-methyl-4-[[(2R)-1-methylazetidin-2-yl]methoxy]pyrazol-3-yl]pyrazolo[1,5-a]pyridin-2-amine. The title compound (17 mg, 2 steps, 5.94%) was prepared as described in

Example 14, except (R)-tert-butyl 2-(((5-(2-((5-fluoropyrimidin-2-yl)amino)pyrazolo[1,5-a]pyridin-5-yl)-1-methyl-1H-pyrazol-4-yl)oxy)methyl)azetidine-1-carboxylate was used instead of tert-butyl (R)-2-(((1-methyl-5-(2-((1R,2R)-2-methylcyclopropane-1-carboxamido)pyrazolo[1,5-a]pyridin-5-yl)-1H-pyrazol-4-yl)oxy)methyl)azetidine-1-carboxylate in Step B and (R)-5-(4-(azetidin-2-ylmethoxy)-1-methyl-1H-pyrazol-5-yl)-N-(5-fluoropyrimidin-2-yl)pyrazolo[1,5-a]pyridin-2-amine was used instead of (1R,2R)—N-(5-(4-(((R)-azetidin-2-yl)methoxy)-1-methyl-1H-pyrazol-5-yl)pyrazolo[1,5-a]pyridin-2-yl)-2-methylcyclopropane-1-carboxamide in Step C. MS (ESI): mass calcd. for C₂₀H₂₁FN₈O, 408.2. m/z found, 409.1 [M+H]⁺. ¹H NMR (400 MHz, DMSO-d6) δ 10.48 (s, 1H), 8.62 (s, 2H), 8.58 (d, J=6.8 Hz, 1H), 7.69 (d, J=1.2 Hz, 1H), 7.45 (s, 1H), 7.06 (s, 1H), 6.93 (dd, J=1.6, 7.2 Hz, 1H), 3.95-3.91 (m, 2H), 3.86 (s, 3H), 3.27-3.19 (m, 2H), 2.73-2.67 (m, 1H), 2.19 (s, 3H), 1.96-1.80 (m, 2H) 19F NMR (376 MHz, DMSO-d6) δ −152.22 (s, 1F)

### Example 396: N-[5-[2-methyl-4-[[(3R)-1-methylpyrrolidin-3-yl]methoxy]pyrazol-3-yl]pyrazolo[1,5-a]pyridin-2-yl]cyclopropanecarboxamide

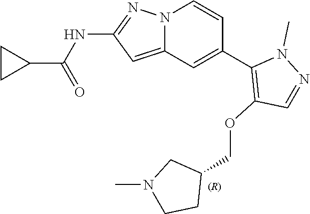

Step A. Tert-butyl (R)-3-(((5-(2-(cyclopropanecarboxamido)pyrazolo[1,5-a]pyridin-5-yl)-1-methyl-1H-pyrazol-4-yl)oxy)methyl)pyrrolidine-1-carboxylate. The title compound was prepared as described in Step B of Example 1, except Intermediate 4 was used instead of (2S,4R)-2-methyl-4-(tosyloxy)pyrrolidine-1-carboxylate and (R)-tert-butyl 3-(bromomethyl)pyrrolidine-1-carboxylate was used instead of 1-methyl-1h-pyrazol-4-ol.

Step B. N-[5-[2-methyl-4-[[(3R)-1-methylpyrrolidin-3-yl]methoxy]pyrazol-3-yl]pyrazolo[1,5-a]pyridin-2-yl]cyclopropanecarboxamide. The title compound (13.4 mg, 2 steps, 12%) was prepared as described in Steps E-F of Example 1, except tert-butyl (R)-3-(((5-(2-(cyclopropanecarboxamido)pyrazolo[1,5-a]pyridin-5-yl)-1-methyl-1H-pyrazol-4-yl)oxy)methyl)pyrrolidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E and (R)—N-(5-(1-methyl-4-(pyrrolidin-3-ylmethoxy)-1H-pyrazol-5-yl)pyrazolo[1,5-a]pyridin-2-yl)cyclopropanecarboxamide was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide in Step F. MS (ESI): mass calcd. for C₂₁H₂₆N₆O₂, 394.2. m/z found, 395.2 [M+H]⁺. ¹H NMR (400 MHz, MeOD-d4): 8.46 (d, J=7.6 Hz, 1H), 7.66-7.57 (m, 1H), 7.43 (s, 1H), 6.94 (dd, J=2.0, 7.2 Hz, 1H), 6.91 (s, 1H), 3.94-3.86 (m, 5H), 2.82-2.73 (m, 1H), 2.69-2.56 (m, 3H), 2.49-2.42 (m, 1H), 2.36 (s, 3H), 2.07-1.95 (m, 1H), 1.92-1.83 (m, 1H), 1.66-1.55 (m, 1H), 1.04-0.97 (m, 2H), 0.94-0.88 (m, 2H)

### Example 397: N-[5-[4-(azetidin-3-yloxy)-2-methyl-pyrazol-3-yl]pyrazolo[1,5-a]pyridin-2-yl]cyclopropanecarboxamide

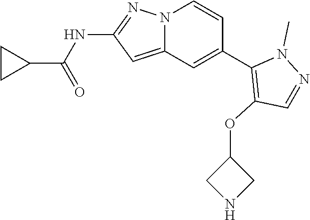 [SMILES: Cn1ncc(OC2CNC2)c1-c1ccn2nc(NC(=O)C3CC3)cc2c1]

Step A. Tert-butyl 3-((5-(2-(cyclopropanecarboxamido)pyrazolo[1,5-a]pyridin-5-yl)-1-methyl-1H-pyrazol-4-yl)oxy)azetidine-1-carboxylate. The title compound was prepared as described in Step B of Example 1, except Intermediate 4 was used instead of (2S,4R)-2-methyl-4-(tosyloxy)pyrrolidine-1-carboxylate and tert-butyl 3-iodoazetidine-1-carboxylate was used instead of 1-methyl-1h-pyrazol-4-ol.

Step B. N-[5-[4-(azetidin-3-yloxy)-2-methyl-pyrazol-3-yl]pyrazolo[1,5-a]pyridin-2-yl]cyclopropanecarboxamide. The title compound (11.1 mg, 15%) was prepared as described in Step E of Example 1, except tert-butyl 3-((5-(2-(cyclopropanecarboxamido)pyrazolo[1,5-a]pyridin-5-yl)-1-methyl-1H-pyrazol-4-yl)oxy)azetidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E and HCl/1,4-dioxane (5 mL) was used instead of TFA/DCM in Step E. MS (ESI): mass calcd. for C₁₈H₂₀N₆O₂, 352.2. m/z found, 353.1 [M+H]⁺. ¹H NMR (400 MHz, DMSO-d6) δ 11.10 (s, 1H), 8.61 (d, J=7.2 Hz, 1H), 7.81-7.60 (m, 1H), 7.28 (s, 1H), 6.95 (dd, J=2.0, 7.2 Hz, 1H), 6.91 (s, 1H), 4.89-4.72 (m, 1H), 3.84 (s, 3H), 3.75-3.48 (m, 4H), 1.99-1.88 (m, 1H), 0.89-0.77 (m, 4H)

### Example 398: N-[5-[2-ethyl-4-[[(3R)-1-methylpyrrolidin-3-yl]methoxy]pyrazol-3-yl]pyrazolo[1,5-a]pyridin-2-yl]cyclopropanecarboxamide

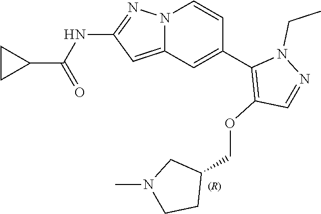

Step A. N-(5-(I-ethyl-4-hydroxy-1H-pyrazol-5-yl)pyrazolo[1,5-a]pyridin-2-yl)cyclopropanecarboxamide. The title compound (650 mg, 125%, impure) was prepared as described in Intermediate 4, except 5-bromo-1-ethyl-1H-pyrazol-4-ol was used instead of 5-bromo-1-methyl-1H-pyrazol-4-ol.

Step B. (R)-tert-butyl 3-((tosyloxy)methyl)pyrrolidine-1-carboxylate. The title compound (41 mg, 87%) was prepared as described in Step A of Intermediate 7, except (R)-tert-butyl 3-(hydroxymethyl)pyrrolidine-1-carboxylate was used instead of (R)-1-N-Boc-3-hydroxypyrrolidine.

Step C. Tert-butyl (R)-3-(((5-(2-(cyclopropanecarboxamido)pyrazolo[1,5-a]pyridin-5-yl)-1-ethyl-1H-pyrazol-4-yl)oxy)methyl)pyrrolidine-1-carboxylate. The title compound (600 mg, 63%) was prepared as described in Step B of Example 1, except N-(5-(1-ethyl-4-hydroxy-1H-pyrazol-5-yl)pyrazolo[1,5-a]pyridin-2-yl)cyclopropanecarboxamide was used instead of (2S,4R)-2-methyl-4-(tosyloxy)pyrrolidine-1-carboxylate in Step B and (R)-tert-butyl 3-((tosyloxy)methyl)pyrrolidine-1-carboxylate was used instead of 1-methyl-1h-pyrazol-4-ol in Step B.

Step D. N-[5-[2-ethyl-4-[[(3R)-1-methylpyrrolidin-3-yl]methoxy]pyrazol-3-yl]pyrazolo[1,5-a]pyridin-2-yl]cyclopropanecarboxamide. The title compound (65.2 mg, 25%, 17% overall) was prepared as described in Step A Example 26, except tert-butyl (R)-3-(((5-(2-(cyclopropanecarboxamido)pyrazolo[1,5-a]pyridin-5-yl)-1-ethyl-1H-pyrazol-4-yl)oxy)methyl)pyrrolidine-1-carboxylate was used instead of tert-butyl (R)-2-(((5-bromo-1-methyl-1H-pyrazol-4-yl)oxy)methyl)azetidine-1-carboxylate in Step A. MS (ESI): mass calcd. for C₂₂H₂₈N₆O₂, 408.2. m/z found, 409.2 [M+H]⁺. ¹H NMR (400 MHz, DMSO-d6) δ 11.11 (s, 1H), 8.61 (d, J=7.2 Hz, 1H), 7.63 (d, J=1.2 Hz, 1H), 7.47 (s, 1H), 6.90 (s, 1H), 6.87 (dd, J=2.0, 6.8 Hz, 1H), 4.13 (q, J=7.2 Hz, 2H), 3.80 (d, J=6.8 Hz, 2H), 2.47-2.37 (m, 3H), 2.36-2.29 (m, 1H), 2.28-2.21 (m, 1H), 2.17 (s, 3H), 1.97-1.89 (m, 1H), 1.88-1.78 (m, 1H), 1.46-1.34 (m, 1H), 1.26 (t, J=6.8 Hz, 3H), 0.88-0.78 (m, 4H)

### Example 399: N-[5-[2-methyl-4-(1-methylazetidin-3-yl)oxy-pyrazol-3-yl]pyrazolo[1,5-a]pyridin-2-yl]cyclopropanecarboxamide

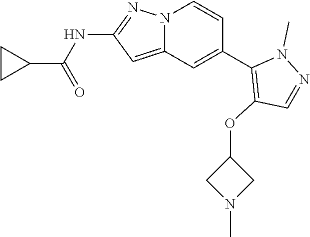 [SMILES: CN1CC(Oc2cnn(C)c2-c2ccn3nc(NC(=O)C4CC4)cc3c2)C1]

Step A. N-(5-(4-(azetidin-3-yloxy)-1-methyl-1H-pyrazol-5-yl)pyrazolo[1,5-a]pyridin-2-yl)cyclopropanecarboxamide. The title compound (150 mg, 2 steps, 84%) was prepared as described in Steps B-C of Example 6, except tert-butyl 3-iodoazetidine-1-carboxylate was used instead of (R)-tert-butyl 2-((tosyloxy)methyl)morpholine-4-carboxylate in Step B and tert-butyl 3-((5-(2-(cyclopropanecarboxamido)pyrazolo[1,5-a]pyridin-5-yl)-1-methyl-1H-pyrazol-4-yl)oxy)azetidine-1-carboxylate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate in Step C.

Step B. N-[5-[2-methyl-4-(1-methylazetidin-3-yl)oxy-pyrazol-3-yl]pyrazolo[1,5-a]pyridin-2-yl]cyclopropanecarboxamide. The title compound (9.3 mg, 4.8%) was prepared as described in Step C of Example 4, except N-(5-(4-(azetidin-3-yloxy)-1-methyl-1H-pyrazol-5-yl)pyrazolo[1,5-a]pyridin-2-yl)cyclopropanecarboxamide was used instead of N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide. MS (ESI): mass calcd. for C₁₉H₂₂N₆O₂, 366.2. m/z found, 367.1 [M+H]⁺. ¹H NMR (400 MHz, DMSO-d6) δ 11.11 (s, 1H), 8.61 (d, J=7.2 Hz, 1H), 7.79-7.62 (m, 1H), 7.32 (s, 1H), 6.94 (dd, J=2.0, 7.2 Hz, 1H), 6.91 (s, 1H), 4.64-4.56 (m, 1H), 3.83 (s, 3H), 3.69-3.60 (m, 2H), 3.01-2.92 (m, 2H), 2.26 (s, 3H), 1.98-1.88 (m, 1H), 0.87-0.78 (m, 4H)

### Example 400: N-[5-[4-[(3-aminocyclobutyl)methoxy]-2-methyl-pyrazol-3-yl]pyrazolo[1,5-a]pyridin-2-yl]cyclopropanecarboxamide

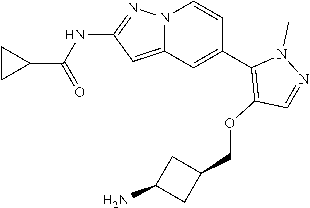 [SMILES: Cn1ncc(OC[C@H]2C[C@@H](N)C2)c1-c1ccn2nc(NC(=O)C3CC3)cc2c1]

Step A. ((1s,3s)-3-((tert-butoxycarbonyl)amino)cyclobutyl)methyl 4-methylbenzenesulfonate. The title compound (400 mg, 91%) was prepared as described in Step A of Intermediate 7, except cis-1-(Boc-amino)-3-(hydroxymethyl)cyclobutane was used instead of (R)-1-N-Boc-3-hydroxypyrrolidine.

Step B. N-[5-[4-[(3-aminocyclobutyl)methoxy]-2-methyl-pyrazol-3-yl]pyrazolo[1,5-a]pyridin-2-yl]cyclopropanecarboxamide. The title compound (7 mg, 2 steps, 3%) was prepared as described in Steps B-C of Intermediate 7, except ((1s,3s)-3-((tert-butoxycarbonyl)amino)cyclobutyl)methyl 4-methylbenzenesulfonate was used instead of (R)-tert-butyl 2-((tosyloxy)methyl)morpholine-4-carboxylate in Step B and tert-butyl ((1s,3s)-3-(((5-(2-(cyclopropanecarboxamido)pyrazolo[1,5-a]pyridin-5-yl)-1-methyl-1H-pyrazol-4-yl)oxy)methyl)cyclobutyl)carbamate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate in Step C. MS (ESI): mass calcd. for C₂₀H₂₄N₆O₂, 380.2. m/z found, 381.2 [M+H]⁺. ¹H NMR (400 MHz, DMSO-d6) δ 11.11 (s, 1H), 8.60 (d, J=6.8 Hz, 1H), 7.69 (d, J=1.2 Hz, 1H), 7.45 (s, 1H), 6.95 (dd, J=2.0, 7.6 Hz, 1H), 6.90 (s, 1H), 3.89 (d, J=6.0 Hz, 2H), 3.85 (s, 3H), 3.48-3.47 (m, 1H), 2.41-2.36 (m, 1H), 2.41-2.36 (m, 1H), 2.28-2.20 (m, 2H), 1.98-1.88 (m, 1H), 1.86-1.74 (m, 2H), 0.86-0.79 (m, 4H)

### Example 401: (*R)—N-[5-[2-methyl-4-(3,3,3-trifluoro-2-hydroxy-propoxy)pyrazol-3-yl]pyrazolo[1,5-a]pyridin-2-yl]cyclopropanecarboxamide

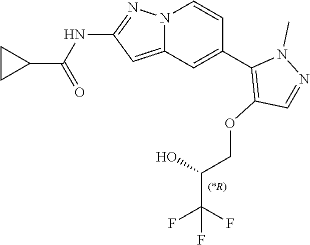

The title compound was prepared as described in Example 6 Step B, except (RS)-2-(trifluoromethyl)oxirane was used instead of (R)-tert-butyl 2-((tosyloxy)methyl)morpholine-4-carboxylate. The title compound was separated by SFC with Phenomenex-Cellulose-2 (250 mm×30 mm, 5 um), column (eluent: 45% to 45% (v/v) MeOH with 0.1% NH₃/H₂O in supercritical CO₂) to afford pure product. The product was suspended in water (10 mL), the mixture frozen using dry ice/EtOH, and then lyophilized to dryness to afford the title compound (63.5 mg, 1.4%) as a white solid. MS (ESI): mass calcd. for C₁₈H₁₈F₃N₅O₃, 409.1. m/z found, 410.2 [M+H]⁺. ¹H NMR (400 MHz, DMSO-d6) δ 11.11 (s, 1H), 8.60 (d, J=7.2 Hz, 1H), 7.72 (s, 1H), 7.52 (s, 1H), 6.98 (dd, J=2.0, 7.6 Hz, 1H), 6.89 (s, 1H), 6.64 (d, J=6.4 Hz, 1H), 4.39-4.26 (m, 1H), 4.19-4.11 (m, 1H), 4.08-3.98 (m, 1H), 3.86 (s, 3H), 1.99-1.89 (m, 1H), 0.88-0.77 (m, 4H) 19F NMR (376 MHz, DMSO-d6):−76.01 (s, 3F)

### Example 402: N-[5-[4-[(3-aminooxetan-3-yl)methoxy]-2-methyl-pyrazol-3-yl]pyrazolo[1,5-a]pyridin-2-yl]cyclopropanecarboxamide

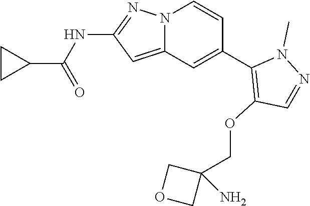 [SMILES: Cn1ncc(OCC2(N)COC2)c1-c1ccn2nc(NC(=O)C3CC3)cc2c1]

Step A. (3-((tert-butoxycarbonyl)amino)oxetan-3-yl)methyl 4-methylbenzenesulfonate. The title compound (580 mg, 66%) was prepared as described in Example 6 Step B, except tert-butyl (3-(hydroxymethyl)oxetan-3-yl)carbamate was used instead of (R)-tert-butyl 2-((tosyloxy)methyl)morpholine-4-carboxylate.

Step B.N-[5-[4-[(3-aminooxetan-3-yl)methoxy]-2-methyl-pyrazol-3-yl]pyrazolo[1,5-a]pyridin-2-yl]cyclopropanecarboxamide. The title compound (12 mg, 2 steps, 7%) was prepared as described in Steps B-C of Example 6, except (3-((tert-butoxycarbonyl)amino)oxetan-3-yl)methyl 4-methylbenzenesulfonate was used instead of (R)-tert-butyl 2-((tosyloxy)methyl)morpholine-4-carboxylate in Step B and tert-butyl (3-(((5-(2-(cyclopropanecarboxamido)pyrazolo[1,5-a]pyridin-5-yl)-1-methyl-1H-pyrazol-4-yl)oxy)methyl)oxetan-3-yl)carbamate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate in Step C.

MS (ESI): mass calcd. for C₁₉H₂₂N₆O₃, 382.2. m/z found, 383.2 [M+H]⁺. ¹H NMR (400 MHz, DMSO-d6) δ 11.09 (s, 1H), 8.59 (d, J=7.2 Hz, 1H), 7.74 (s, 1H), 7.51 (s, 1H), 6.99 (dd, J=2.0, 7.2 Hz, 1H), 6.89 (s, 1H), 4.41 (d, J=6.0 Hz, 2H), 4.31 (d, J=6.0 Hz, 2H), 4.01 (s, 2H), 3.87 (s, 3H), 2.24 (br s, 2H), 1.97-1.89 (m, 1H), 0.89-0.77 (m, 4H)

### Example 403: N-[5-[4-[(2R)-2-hydroxy-2-phenyl-ethoxy]-2-methyl-pyrazol-3-yl]pyrazolo[1,5-a]pyridin-2-yl]cyclopropanecarboxamide

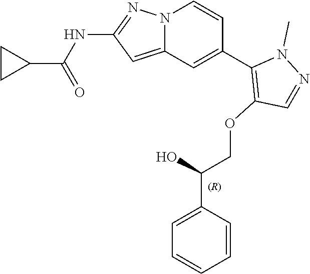

The title compound (41.4 mg, 15%) was prepared as described in Step B of Example 6, except (R)-2-phenyloxirane was used instead of (R)-tert-butyl 2-((tosyloxy)methyl)morpholine-4-carboxylate in Step B. MS (ESI): mass calcd. for C₂₃H₂₃N₅O₃, 417.2. m/z found, 418.2 [M+H]⁺. ¹H NMR (400 MHz, DMSO-d6) δ 11.09 (s, 1H), 8.53 (d, J=7.6 Hz, 1H), 7.62 (s, 1H), 7.44 (s, 1H), 7.42-7.37 (m, 2H), 7.35-7.25 (m, 3H), 6.91 (dd, J=1.6, 7.2 Hz, 1H), 6.88 (s, 1H), 5.61 (s, 1H), 4.86 (t, J=5.6 Hz, 1H), 4.07-3.95 (m, 2H), 3.85 (s, 3H), 1.99-1.88 (m, 1H), 0.89-0.80 (m, 4H)

### Example 404: N-[5-[4-(4-hydroxycyclohexoxy)-2-methyl-pyrazol-3-yl]pyrazolo[1,5-a]pyridin-2-yl]cyclopropanecarboxamide

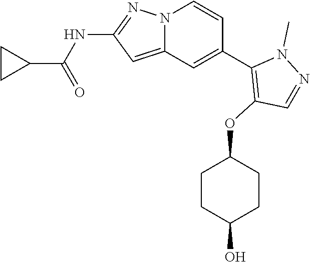 [SMILES: Cn1ncc(O[C@H]2CC[C@@H](O)CC2)c1-c1ccn2nc(NC(=O)C3CC3)cc2c1]

Step A. 4-hydroxycyclohexyl 4-methylbenzenesulfonate. To a solution consisting of (trans)-cyclohexane-1,4-diol (1.00 g, 8.61 mmol) and DCM (20 mL) was cooled to 0° C., then add TsCl (1.81 g, 9.47 mmol), TEA (1.74 g, 17.2 mmol) and DMAP (0.105 mg, 0.861 mmol) to the mixture. The reaction was stirred at 35° C. for 2 h. The reaction mixture was concentrated to dryness under reduced pressure to give the crude product, which was purified by FCC (eluent: petroleum ether:ethyl acetate=1:0 to 1:1) to afford the title compound (1.1 g, 47%) as a light yellow solid.

Step B. N-[5-[4-(4-hydroxycyclohexoxy)-2-methyl-pyrazol-3-yl]pyrazolo[1,5-a]pyridin-2-yl]cyclopropanecarboxamide. To a solution consisting of N-(5-(4-hydroxy-1-methyl-1H-pyrazol-5-yl)pyrazolo[1,5-a]pyridin-2-yl)cyclopropanecarboxamide (150 mg, 0.505 mmol), (trans)-4-hydroxycyclohexyl 4-methylbenzenesulfonate (273 mg, 1.01 mmol), Cs₂CO₃ (493 mg, 1.51 mmol) and DMF (1 mL) was stirred at microwave 80° C. for 2 h. The reaction mixture was purified by preparative HPLC using a HT C18 Highload 150×25 mm×5 um column (eluent: 18% to 38% (v/v) CH₃CN and H₂O with 0.04% NH₃) to afford pure product. The product was suspended in water (10 mL), the mixture frozen using dry ice/ethanol, and then lyophilized to dryness to afford the title compound as a white solid (11.20 mg, 2%). MS (ESI): mass calcd. for C₂₁H₂₅N₅O₃, 395.2. m/z found, 396.2 [M+H]⁺. ¹H NMR (400 MHz, DMSO-d6) δ 11.11 (s, 1H), 8.60 (d, J=7.2 Hz, 1H), 7.74-7.66 (m, 1H), 7.41 (s, 1H), 6.99 (dd, J=2.0, 7.2 Hz, 1H), 6.90 (s, 1H), 4.45 (d, J=3.2 Hz, 1H), 4.10-4.02 (m, 1H), 3.85 (s, 3H), 3.56-3.46 (m, 1H), 1.98-1.87 (m, 1H), 1.85-1.73 (m, 2H), 1.60-1.39 (m, 6H), 0.91-0.75 (m, 4H)

### Example 405: N-[5-[4-[(3-fluoro-1-methyl-azetidin-3-yl)methoxy]-2-methyl-pyrazol-3-yl]pyrazolo[1,5-a]pyridin-2-yl]cyclopropanecarboxamide

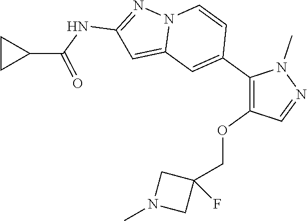 [SMILES: CN1CC(F)(COc2cnn(C)c2-c2ccn3nc(NC(=O)C4CC4)cc3c2)C1]

Step A. Tert-butyl 3-fluoro-3-((tosyloxy)methyl)azetidine-1-carboxylate. The title compound (300 mg, 34%) was prepared as described in Example 404 Step A, except tert-butyl 3-fluoro-3-(hydroxymethyl)azetidine-1-carboxylate was used instead of (trans)-cyclohexane-1,4-diol.

Step B. Tert-butyl 3-(((5-(2-(cyclopropanecarboxamido)pyrazolo[1,5-a]pyridin-5-yl)-1-methyl-1H-pyrazol-4-yl)oxy)methyl)-3-fluoroazetidine-1-carboxylate. The title compound (700 mg, 215%, impure) was prepared as described in Example 404 Step B, except tert-butyl 3-fluoro-3-((tosyloxy)methyl)azetidine-1-carboxylate was used instead of (trans)-4-hydroxycyclohexyl 4-methylbenzenesulfonate.

Step C. N-[5-[4-[(3-fluoro-1-methyl-azetidin-3-yl)methoxy]-2-methyl-pyrazol-3-yl]pyrazolo[1,5-a]pyridin-2-yl]cyclopropanecarboxamide. The title compound (22.8 mg, 2 steps, 5%) was prepared as described in Steps B-C of Example 17, except tert-butyl 3-(((5-(2-(cyclopropanecarboxamido)pyrazolo[1,5-a]pyridin-5-yl)-1-methyl-1H-pyrazol-4-yl)oxy)methyl)-3-fluoroazetidine-1-carboxylate was used instead of tert-butyl (R)-2-(((5-(2-((5,6-dimethylpyridazin-3-yl)amino)pyrazolo[1,5-a]pyridin-5-yl)-1-methyl-1H-pyrazol-4-yl)oxy)methyl)azetidine-1-carboxylate in Step B and N-(5-(4-((3-fluoroazetidin-3-yl)methoxy)-1-methyl-1H-pyrazol-5-yl)pyrazolo[1,5-a]pyridin-2-yl)cyclopropanecarboxamide was used instead of (R)-5-(4-(azetidin-2-ylmethoxy)-1-methyl-1H-pyrazol-5-yl)-N-(5,6-dimethylpyridazin-3-yl)pyrazolo[1,5-a]pyridin-2-amine in Step C. MS (ESI): mass calcd. for C₂₀H₂₃FN₆O₂, 398.2. m/z found, 399.1 [M+H]⁺. ¹H NMR (400 MHz, DMSO-d6) δ 11.15 (s, 1H), 8.62 (d, J=7.2 Hz, 1H), 7.71 (d, J=0.8 Hz, 1H), 7.52 (s, 1H), 6.94 (dd, J=2.0, 7.2 Hz, 1H), 6.90 (s, 1H), 4.24 (d, J=24.8 Hz, 2H), 3.86 (s, 3H), 3.50-3.44 (m, 2H), 3.14-2.99 (m, 2H), 2.28 (s, 3H), 1.99-1.87 (m, 1H), 0.92-0.78 (m, 4H)

### Example 406: N-[5-[4-[(3-methoxy-1-methyl-azetidin-3-yl)methoxy]-2-methyl-pyrazol-3-yl]pyrazolo[1,5-a]pyridin-2-yl]cyclopropanecarboxamide

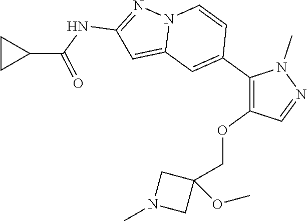 [SMILES: COC1(COc2cnn(C)c2-c2ccn3nc(NC(=O)C4CC4)cc3c2)CN(C)C1]

Step A. N-(5-(4-((3-methoxyazetidin-3-yl)methoxy)-1-methyl-1H-pyrazol-5-yl)pyrazolo[1,5-a]pyridin-2-yl)cyclopropanecarboxamide. The title compound (1 g, 36%, impure) was prepared as described in Step A, B, D of Example 386, except tert-butyl 3-(hydroxymethyl)-3-methoxyazetidine-1-carboxylate was used instead of tert-butyl-3-hydroxycyclobutylcarbamate in Step A, tert-butyl 3-methoxy-3-((tosyloxy)methyl)azetidine-1-carboxylate was used instead of (cis)-3-((tert-butoxycarbonyl)amino)cyclobutyl 4-methylbenzenesulfonate in Step B, cesium carbonate was used instead of K₂CO₃ in Step B, and DMF (5 mL) was used instead of CH₃CN in Step B and tert-butyl 3-(((5-(2-(cyclopropanecarboxamido)pyrazolo[1,5-a]pyridin-5-yl)-1-methyl-1H-pyrazol-4-yl)oxy)methyl)-3-methoxyazetidine-1-carboxylate was used instead of tert-butyl ((trans)-3-((4-(2-(cyclopropanecarboxamido)pyrazolo[1,5-a]pyridin-5-yl)-5-methylisoxazol-3-yl)oxy)cyclobutyl)carbamate in Step D.

Step B. N-[5-[4-[(3-methoxy-1-methyl-azetidin-3-yl)methoxy]-2-methyl-pyrazol-3-yl]pyrazolo[1,5-a]pyridin-2-yl]cyclopropanecarboxamide. The title compound (33.6 mg, 12%) was prepared as described in Example 1 Step F, except N-(5-(4-((3-methoxyazetidin-3-yl)methoxy)-1-methyl-1H-pyrazol-5-yl)pyrazolo[1,5-a]pyridin-2-yl)cyclopropanecarboxamide was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₁H₂₆N₆O₃, 410.2. m/z found, 411.1 [M+H]⁺. ¹H NMR (400 MHz, DMSO-d6) δ 11.11 (s, 1H), 8.61 (d, J=7.2 Hz, 1H), 7.71 (s, 1H), 7.51 (s, 1H), 6.96 (dd, J=2.0, 7.2 Hz, 1H), 6.88 (s, 1H), 4.15 (s, 2H), 3.86 (s, 3H), 3.26 (d, J=8.0 Hz, 2H), 3.14 (s, 3H), 2.88 (d, J=8.4 Hz, 2H), 2.23 (s, 3H), 2.00-1.87 (m, 1H), 0.88-0.78 (m, 4H)

### Example 407: N-[5-[2-methyl-4-(4-piperidyloxy)pyrazol-3-yl]pyrazolo[1,5-a]pyridin-2-yl]cyclopropanecarboxamide

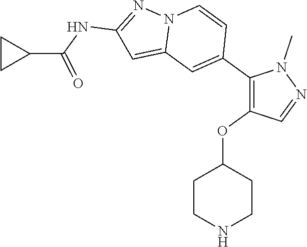 [SMILES: Cn1ncc(OC2CCNCC2)c1-c1ccn2nc(NC(=O)C3CC3)cc2c1]

To a 100 mL round bottom flask charged with TPP (176.439 mg, 0.673 mmol), tert-butyl 4-hydroxypiperidine-1-carboxylate (135.388 mg, 0.673 mmol) and Intermediate 4 (100 mg, 0.336 mmol) was added THE (1.682 mL, 0.2 M, 0.336 mmol). The solid was allowed to dissolved and then cooled to 0° C. for 15 min. To the resulting solution was added DIAD (0.132 mL, 1.027 g/mL, 0.673 mmol) in slow drops. After addition of the DIAD, the ice-bath was removed and the solution was heated to 60° C. for 10 hr. The reaction was stirred at 50° C. overnight. The crude was diluted with EtOAc and washed with brine 1×. The crude was absorbed onto silica gel and flashed with 50-100% EtOAc:Hex. PPh₃O was eluted first, characterized by difference in UV absorption. Then, the desired material was eluted. The fractions contained desired material was collected, even with some PPh₃O contamination, and concentrated down. To crude clear residue was added DCM (0.862 mL, 1.326 g/mL, 13.454 mmol) and TFA (0.257 mL, 1.49 g/mL, 3.363 mmol). The reaction was complete after 1 h at room temperature. The crude was evaporated in vacuo. The crude oil residue was dissolved with DCM/MeOH and concentrated down again. The crude was dissolved with DMSO and purified via ISCO prep HPLC using 10-60% ACN-H₂O with 20 MM of NH₃OH as modifier. The fractions contained desired material was collected and concentrated down to −30 mL under vacuo at which point white precipitates formed. The mixture was transferred to 20 mL flask, freeze, and lyophilized down to afford 46 mg, 36% of product. MS (ESI): mass calcd. for C₂₀H₂₄N₆O₂, 380.2. m/z found, 381.1 [M+H]⁺. ¹H NMR (500 MHz, DMSO-d6) δ 11.00 (s, 1H), 8.53 (dd, J=7.2, 1.0 Hz, 1H), 7.61 (dd, J=2.1, 0.9 Hz, 1H), 7.36 (s, 1H), 6.89 (dd, J=7.2, 2.0 Hz, 1H), 6.82 (s, 1H), 3.95 (dt, J=8.8, 4.6 Hz, 1H), 3.77 (s, 3H), 2.76 (dt, J=12.1, 4.4 Hz, 2H), 2.36 (ddd, J=12.6, 9.5, 3.1 Hz, 2H), 1.86 (dq, J=7.6, 4.9, 3.9 Hz, 1H), 1.78-1.69 (m, 2H), 1.31 (dtd, J=12.7, 9.0, 3.7 Hz, 2H), 0.83-0.68 (m, 4H).

### Example 408: N-[5-[4-[(3-ethyl-1-methyl-azetidin-3-yl)methoxy]-2-methyl-pyrazol-3-yl]pyrazolo[1,5-a]pyridin-2-yl]cyclopropanecarboxamide

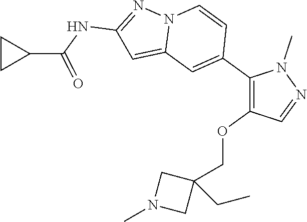 [SMILES: CCC1(COc2cnn(C)c2-c2ccn3nc(NC(=O)C4CC4)cc3c2)CN(C)C1]

The title compound (26.6 mg, 4 steps, 6%) was prepared as described in Example 404 (Steps A-B) and Example 1 (Steps E-F), except tert-butyl 3-ethyl-3-(hydroxymethyl)azetidine-1-carboxylate was used instead of (trans)-cyclohexane-1,4-diol in Step A, tert-butyl 3-ethyl-3-((tosyloxy)methyl)azetidine-1-carboxylate was used instead of (trans)-4-hydroxycyclohexyl 4-methylbenzenesulfonate in Step B, (R)-tert-butyl 3-(((5-(2-(cyclopropanecarboxamido)pyrazolo[1,5-a]pyridin-5-yl)-1-((1-methyl-1H-pyrazol-4-yl)methyl)-1H-pyrazol-4-yl)oxy)methyl)pyrrolidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E, and N-(5-(4-((3-ethylazetidin-3-yl)methoxy)-1-methyl-1H-pyrazol-5-yl)pyrazolo[1,5-a]pyridin-2-yl)cyclopropanecarboxamide was used instead of N-(5-(1-methyl-4-(((3 S,5S)-5-methylpyrrolidin-3-yl)oxy)-1H-pyrazol-5-yl)pyrazolo[1,5-a]pyridin-2-yl)cyclopropanecarboxamide in Step F. MS (ESI): mass calcd. for C₂₂H₂₈N₆O₂, 408.2. m/z found, 409.3 [M+H]⁺. ¹H NMR (400 MHz, DMSO-d6) δ 11.12 (s, 1H), 8.61 (d, J=7.2 Hz, 1H), 7.70 (d, J=1.2 Hz, 1H), 7.49 (s, 1H), 6.96 (dd, J=2.0, 7.2 Hz, 1H), 6.88 (s, 1H), 3.95 (s, 2H), 3.87 (s, 3H), 3.01 (d, J=6.8 Hz, 2H), 2.82 (d, J=7.2 Hz, 2H), 2.16 (s, 3H), 1.98-1.89 (m, 1H), 1.61 (q, J=7.6 Hz, 2H), 0.84-0.76 (m, 7H)

### Example 409: (rac-cis)-N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide

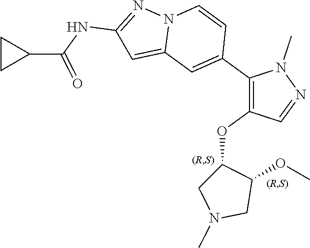 [SMILES: CO[C@@H]1CN(C)C[C@@H]1Oc1cnn(C)c1-c1ccn2nc(NC(=O)C3CC3)cc2c1]

The title compound (72.6 mg, 5 steps, 4%) was prepared as described in Example 1, except rac-tert-butyl (3r,4r)-3-hydroxy-4-methoxypyrrolidine-1-carboxylate, trans was used instead of (2s,4r)-tert-butyl 4-hydroxy-2-methylpyrrolidine-1-carboxylate in Step A, tert-butyl (3R,4S)-3-methoxy-4-((1-methyl-1H-pyrazol-4-yl)oxy)pyrrolidine-1-carboxylate was used instead of (2S,4R)-2-methyl-4-(tosyloxy)pyrrolidine-1-carboxylate in Step B, rac-tert-butyl (3R,4S)-3-((5-bromo-1-methyl-1H-pyrazol-4-yl)oxy)-4-methoxypyrrolidine-1-carboxylate was used instead of tert-butyl (2S,4S)-2-methyl-4-((1-methyl-1H-pyrazol-4-yl)oxy)pyrrolidine-1-carboxylate in Step C, rac-tert-butyl (3R,4S)-3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate was used instead of (2S,4S)-4-((5-bromo-1-methyl-1H-pyrazol-4-yl)oxy)-2-methylpyrrolidine-1-carboxylate in Step D, and rac-N-(5-(4-(((3R,4S)-4-methoxypyrrolidin-3-yl)oxy)-1-methyl-1H-pyrazol-5-yl)pyrazolo[1,5-a]pyridin-2-yl)cyclopropanecarboxamide was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E. MS (ESI): mass calcd. for C₂₁H₂₆N₆O₃, 410.2. m/z found, 411.2 [M+H]⁺. ¹H NMR (500 MHz, Methanol-d4) δ 8.49-8.37 (m, 1H), 7.77-7.64 (m, 1H), 7.44 (s, 1H), 7.03 (dd, J=7.2, 1.9 Hz, 1H), 6.91 (s, 1H), 4.67-4.60 (m, 1H), 4.00-3.95 (m, 1H), 3.88 (s, 3H), 3.31 (s, 3H), 3.03-2.95 (m, 1H), 2.92-2.84 (m, 1H), 2.80-2.75 (m, 1H), 2.73-2.68 (m, 1H), 2.31 (s, 3H), 1.93-1.84 (m, 1H), 1.04-0.97 (m, 2H), 0.93-0.86 (m, 2H).

### Example 410: N-[5-[2-methyl-4-[(1-methyl-3-phenyl-azetidin-3-yl)methoxy]pyrazol-3-yl]pyrazolo[1,5-a]pyridin-2-yl]cyclopropanecarboxamide

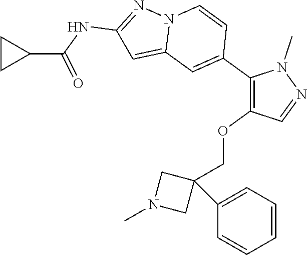 [SMILES: CN1CC(COc2cnn(C)c2-c2ccn3nc(NC(=O)C4CC4)cc3c2)(c2ccccc2)C1]

Step A. Tert-butyl 3-(hydroxymethyl)-3-phenylazetidine-1-carboxylate. To a 50 mL round bottom flask was added (3-phenylazetidin-3-yl)methanol hydrochloride (1088 mg, 5.449 mmol) as a solid followed by DCM (18 mL, 5.449 mmol) then DIPEA (2.817 mL, 16.346 mmol). To the mixture was added a solution of di-tert-butyl dicarbonate (1391 mg, 6.31 mmol, 1.158 mmol) in DCM dropwise. The reaction was subjected to an aqueous workup and then diluted with DCM. The solution was washed with water (2×50 mL) then dried with Na₂SO₄ and filtered then concentrated to dryness to afford 1409 mg, 98% of impure material.

Step B. N-[5-[2-methyl-4-[(1-methyl-3-phenyl-azetidin-3-yl)methoxy]pyrazol-3-yl]pyrazolo[1,5-a]pyridin-2-yl]cyclopropanecarboxamide. The title compound (38 mg, 4 steps, 4 steps, 35%) was prepared as described in Example 1 (Steps A, B, E, F) except tert-butyl 3-(hydroxymethyl)-3-phenylazetidine-1-carboxylate was used instead of (2s,4r)-tert-butyl 4-hydroxy-2-methylpyrrolidine-1-carboxylate in Step A, tert-butyl 3-phenyl-3-((tosyloxy)methyl)azetidine-1-carboxylate was used instead of (2S,4R)-2-methyl-4-(tosyloxy)pyrrolidine-1-carboxylate in Step B, tert-butyl 3-(((5-(2-(cyclopropanecarboxamido)pyrazolo[1,5-a]pyridin-5-yl)-1-methyl-1H-pyrazol-4-yl)oxy)methyl)-3-phenylazetidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E, and N-(5-(1-methyl-4-((3-phenylazetidin-3-yl)methoxy)-1H-pyrazol-5-yl)pyrazolo[1,5-a]pyridin-2-yl)cyclopropanecarboxamide was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide in Step F. MS (ESI): mass calcd. for C₂₆H₂₈N₆O₂, 456.2. m/z found, 457.4 [M+H]⁺. ¹H NMR (400 MHz, DMSO-d6) δ 11.07 (s, 1H), 8.51 (dt, J=7.2, 1.0 Hz, 1H), 7.53 (dd, J=2.0, 1.0 Hz, 1H), 7.38 (s, 1H), 7.33-7.26 (m, 2H), 7.24-7.14 (m, 3H), 6.88 (s, 1H), 6.79 (dd, J=7.2, 2.0 Hz, 1H), 4.25 (s, 2H), 3.83 (s, 3H), 3.57-3.49 (m, 2H), 3.19 (d, J=7.2 Hz, 2H), 2.20 (s, 3H), 1.94 (dp, J=7.7, 4.8 Hz, 1H), 0.91-0.72 (m, 4H).

### Example 411: (3*R,4*S)—N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide

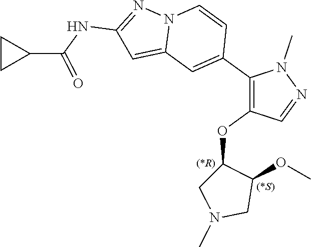 [SMILES: CO[C@H]1CN(C)C[C@H]1Oc1cnn(C)c1-c1ccn2nc(NC(=O)C3CC3)cc2c1]

The title compound (21.4 mg, 1%) was prepared as described in Example 409, except the title compound was the second to elute at 7.15 min. The preparative method used was AD-H (2×25 cm) 30% ethanol (0.1% DEA)/CO₂, 100 bar 60 mL/min, 260 nm injection vol.: 0.5 mL, 6 mg/mL methanol.

MS (ESI): mass calcd. for C₂₁H₂₆N₆O₃, 410.2. m/z found, 411.2 [M+H]⁺. ¹H NMR (400 MHz, Methanol-d4) δ 8.46-8.43 (m, 1H), 7.72-7.69 (m, 1H), 7.44 (s, 1H), 7.03 (dd, J=7.2, 1.9 Hz, 1H), 6.91 (s, 1H), 4.69-4.60 (m, 1H), 4.01-3.95 (m, 1H), 3.89 (s, 3H), 3.31 (s, 3H), 3.00-2.95 (m, 1H), 2.91-2.85 (m, 1H), 2.76 (dd, J=11.0, 4.2 Hz, 1H), 2.70 (dd, J=10.1, 5.8 Hz, 1H), 2.31 (s, 3H), 1.91-1.83 (m, 1H), 1.02-0.98 (m, 2H), 0.92-0.89 (m, 2H).

### Example 412: N-[5-[2-methyl-4-[[(2S)-pyrrolidin-2-yl]methoxy]pyrazol-3-yl]pyrazolo[1,5-a]pyridin-2-yl]cyclopropanecarboxamide

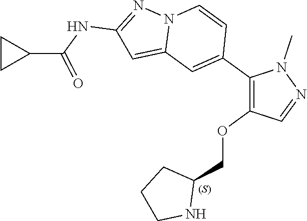

The title compound (30 mg, 39%) was prepared as described in Example 26, except tert-butyl (S)-2-(hydroxymethyl)pyrrolidine-1-carboxylate was used instead of tert-butyl 4-hydroxypiperidine-1-carboxylate. MS (ESI): mass calcd. for C₂₀H₂₄N₆O₂, 380.2. m/z found, 381.2 [M+H]⁺. ¹H NMR (500 MHz, Methanol-d4) δ 8.51-8.37 (m, 1H), 7.64 (t, J=1.4 Hz, 1H), 7.43 (s, 1H), 6.96 (dd, J=7.2, 2.0 Hz, 1H), 6.91 (s, 1H), 3.97 (dd, J=9.6, 4.6 Hz, 1H), 3.95-3.88 (m, 1H), 3.87 (s, 3H), 3.49-3.39 (m, 1H), 3.01-2.78 (m, 2H), 1.99-1.82 (m, 2H), 1.82-1.67 (m, 2H), 1.61-1.45 (m, 1H), 1.05-0.94 (m, 2H), 0.90 (dt, J=8.1, 3.3 Hz, 2H).

### Example 413: (3*S,4*S)—N-[5-[2-methyl-4-(4-methylpyrrolidin-3-yl)oxy-pyrazol-3-yl]pyrazolo[1,5-a]pyridin-2-yl]cyclopropanecarboxamide

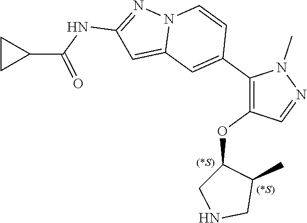 [SMILES: C[C@H]1CNC[C@H]1Oc1cnn(C)c1-c1ccn2nc(NC(=O)C3CC3)cc2c1]

The title compound (29.6 mg, 3 steps, 3%) was prepared as described in Steps A, B, and E of Example 1, except (trans)-tert-butyl 3-hydroxy-4-methylpyrrolidine-1-carboxylate was used instead of (2s,4r)-tert-butyl 4-hydroxy-2-methylpyrrolidine-1-carboxylate in Step A and (3S,4R)-tert-butyl 3-methyl-4-(tosyloxy)pyrrolidine-1-carboxylate was used instead of (2S,4R)-2-methyl-4-(tosyloxy)pyrrolidine-1-carboxylate in Step B. After Step B the reaction was further purified by instrument Phenomenex-Cellulose-2 250×30 mm×5 μm column (eluent: 40% to 40% (v/v) 0.10% NH₃H₂O MeOH in supercritical CO₂. The pure fractions were collected and the volatiles were removed under reduced pressure. The product was suspended in water (300 mL), the mixture frozen using dry ice/EtOH, and then lyophilized to dryness to afford tert-butyl (3S,4S)-3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate as a yellow solid (150 mg, 32%). The title compound was further prepared as described in Step E of Example 1, except tert-butyl (3S,4S)-3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate as was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate. MS (ESI): mass calcd. for C₂₀H₂₄N₆O₂, 380.2. m/z found, 381.2 [M+H]⁺. ¹H NMR (400 MHz, CD3OD): 8.49-8.40 (m, 1H), 7.66-7.56 (m, 1H), 7.43-7.39 (m, 1H), 6.97-6.87 (m, 2H), 4.61-4.46 (m, 1H), 3.94-3.82 (m, 3H), 3.69-3.39; 3.28-3.15 (m, 3H), 3.11-3.01; 2.82-2.69 (m, 1H), 2.43-2.23 (m, 1H), 1.95-1.81 (m, 1H), 1.13-1.07 (m, 3H), 1.03-0.96 (m, 2H), 0.95-0.86 (m, 2H) 1H NMR (400 MHz, DMSO-d6) δ 11.11 (s, 1H), 8.63-8.55 (m, 1H), 7.73-7.62 (m, 1H), 7.47-7.36 (m, 1H), 7.01-6.90 (m, 1H), 6.89 (s, 1H), 4.58-4.35 (m, 1H), 3.86 (s, 3H), 3.41-3.39 (m, 1H), 3.14-2.80 (m, 2H), 2.49-2.08 (m, 2H), 1.98-1.89 (m, 1H), 1.04-0.96 (m, 3H), 0.87-0.79 (m, 4H)

### Example 414: (3*R,4*R)—N-[5-[4-(4-hydroxypyrrolidin-3-yl)oxy-2-methyl-pyrazol-3-yl]pyrazolo[1,5-a]pyridin-2-yl]cyclopropanecarboxamide

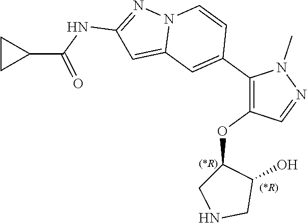 [SMILES: Cn1ncc(O[C@@H]2CNC[C@H]2O)c1-c1ccn2nc(NC(=O)C3CC3)cc2c1]

The title compound (19.5 mg, 3 steps, 9%) was prepared as described in Example 5 (Steps C-E), except (trans-rel)-tert-butyl 3-hydroxy-4-((1-methyl-1H-pyrazol-4-yl)oxy)pyrrolidine-1-carboxylate was used instead of rac-tert-butyl (3r,4s)-3-methoxy-4-((1-methyl-1H-pyrazol-4-yl)oxy)pyrrolidine-1-carboxylate in Step C and (trans-rel)-tert-butyl 3-((5-bromo-1-methyl-1H-pyrazol-4-yl)oxy)-4-hydroxypyrrolidine-1-carboxylate was used instead of rac-cis-tert-butyl (3s,4r)-3-((5-bromo-1-methyl-1H-pyrazol-4-yl)oxy)-4-methoxypyrrolidine-1-carboxylate in Step D and (trans-rel)-tert-butyl 3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate was used instead of rac-cis-tert-butyl (3s,4r)-3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate in Step E. After Step E, the compound was further purified by preparative HPLC with Boston Prime C18 150×30 mm×5 um, column (eluent: 15% to 45% (v/v) CH₃CN and H₂O with 0.04% NH₃H₂₀+10 mM NH₄HCO₃) to afford product. The product was further purified by SFC with DAICEL CHIRALPAK AD(250 mm×30 mm, 10 um) column (eluent: 50% to 50% (v/v) EtOH with 0.01% NH₃H₂O) to afford product. The product was suspended in water (10 mL), the mixture frozen using dry ice/EtOH, and then lyophilized to dryness to afford the title compound (17.1 mg, 14%) as a white solid. MS (ESI): mass calcd. for C₁₉H₂₂N₆O₃, 382.2. m/z found, 383.3 [M+H]⁺. ¹H NMR (400 MHz, DMSO-d6) δ 11.09 (s, 1H), 8.63-8.52 (m, 1H), 7.74-7.61 (m, 1H), 7.54-7.44 (m, 1H), 6.98-6.85 (m, 2H), 5.48-5.03 (m, 1H), 4.45-4.24 (m, 1H), 4.18-4.02 (m, 1H), 3.85 (s, 3H), 3.19-3.08 (m, 1H), 2.99-2.89 (m, 1H), 2.86-2.76 (m, 1H), 2.65-2.59 (m, 1H), 1.98-1.88 (m, 1H), 0.88-0.78 (m, 4H)

### Example 415: N-[5-[4-[(3R,4S)-4-fluoro-1-methyl-pyrrolidin-3-yl]oxy-2-methyl-pyrazol-3-yl]pyrazolo[1,5-a]pyridin-2-yl]cyclopropanecarboxamide

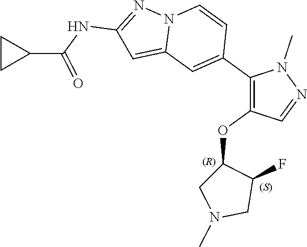

Step A. (3S,4S)-tert-butyl 3-fluoro-4-((methylsulfonyl)oxy)pyrrolidine-1-carboxylate. The title compound (300 mg, 8%) was prepared as described in Step A of Example 5, except (3S,4S)-tert-butyl 3-fluoro-4-hydroxypyrrolidine-1-carboxylate was used instead of (3r,4r)-3-hydroxy-4-methoxypyrrolidine-1-carboxylate and methanesulfonyl chloride was used instead of 4-toluenesulfonyl chloride.

Step B. (3R,4S)-tert-butyl 3-((5-(2-(cyclopropanecarboxamido)pyrazolo[1,5-a]pyridin-5-yl)-1-methyl-1H-pyrazol-4-yl)oxy)-4-fluoropyrrolidine-1-carboxylate. The title compound (300 mg, 8%) was prepared as described in Step B of Example 5, except (3S,4S)-tert-butyl 3-fluoro-4-((methylsulfonyl)oxy)pyrrolidine-1-carboxylate was used instead of rac-trans-tert-butyl (3r,4r)-3-methoxy-4-(tosyloxy)pyrrolidine-1-carboxylate.

Step C. N-[5-[4-[(3R,4S)-4-fluoro-1-methyl-pyrrolidin-3-yl]oxy-2-methyl-pyrazol-3-yl]pyrazolo[1,5-a]pyridin-2-yl]cyclopropanecarboxamide. The title compound (17.3 mg, 1.48%) was prepared as described in Example 26 Step A, except N-[5-[4-[(3R,4S)-4-fluoro-1-methyl-pyrrolidin-3-yl]oxy-2-methyl-pyrazol-3-yl]pyrazolo[1,5-a]pyridin-2-yl]cyclopropanecarboxamide was used instead of tert-butyl (R)-2-(((5-bromo-1-methyl-1H-pyrazol-4-yl)oxy)methyl)azetidine-1-carboxylate. The reaction mixture was purified by preparative HPLC using a YMC-Triart Prep C18, 250×50 mm×10 μm column (eluent: 25% to 55% (v/v) CH₃CN and NH₃H₂O with 10 mM NH₄HCO₃) to afford pure product. The product was suspended in water (10 mL), the mixture frozen using dry ice/acetone, and then lyophilized to dryness to afford the title compound as yellow solid. MS (ESI): mass calcd. for C₂₀H₂₃FN₆O₂, 398.2. m/z found, 399.39 [M+H]⁺.

### Example 416: N-[5-[4-[[(3S,4S)-4-hydroxypyrrolidin-3-yl]methoxy]-2-methyl-pyrazol-3-yl]pyrazolo[1,5-a]pyridin-2-yl]cyclopropanecarboxamide

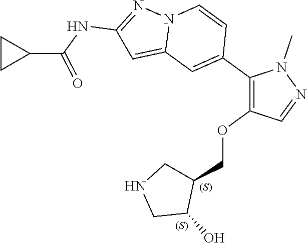

Step A. (3S,4S)-tert-butyl 3-(((5-(2-(cyclopropanecarboxamido)pyrazolo[1,5-a]pyridin-5-yl)-1-methyl-1H-pyrazol-4-yl)oxy)methyl)-4-hydroxypyrrolidine-1-carboxylate. The title compound (300 mg, 2 steps, 17%) was prepared as described in Example 404, Steps A-B, except (3S,4S)-tert-butyl 3-hydroxy-4-(hydroxymethyl)pyrrolidine-1-carboxylate was used instead of (trans)-cyclohexane-1,4-diol in Step A and (3S,4S)-tert-butyl 3-hydroxy-4-((tosyloxy)methyl)pyrrolidine-1-carboxylate was used instead of (trans)-4-hydroxycyclohexyl 4-methylbenzenesulfonate in Step B.

Step B. N-[5-[4-[[(3S,4S)-4-hydroxypyrrolidin-3-yl]methoxy]-2-methyl-pyrazol-3-yl]pyrazolo[1,5-a]pyridin-2-yl]cyclopropanecarboxamide. The title compound (27.2 mg, 23%) was prepared as described by Example 6 Step C, except (3S,4S)-tert-butyl 3-(((5-(2-(cyclopropanecarboxamido)pyrazolo[1,5-a]pyridin-5-yl)-1-methyl-1H-pyrazol-4-yl)oxy)methyl)-4-hydroxypyrrolidine-1-carboxylate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate. MS (ESI): mass calcd. for C₂₀H₂₄N₆O₃, 396.2. m/z found, 397.3 [M+H]⁺. ¹H NMR (400 MHz, DMSO-d6) δ 11.10 (s, 1H), 8.65-8.52 (m, 1H), 7.69 (d, J=0.4 Hz, 1H), 7.48-7.42 (m, 1H), 6.99-6.92 (m, 1H), 6.89 (s, 1H), 5.22-4.65 (m, 1H), 4.12-3.74 (m, 6H), 3.46-3.42 (m, 1H), 3.16-3.05 (m, 1H), 3.02-2.92 (m, 1H), 2.88-2.77 (m, 1H), 2.31-2.08 (m, 1H), 1.97-1.89 (m, 1H), 0.88-0.76 (m, 4H)

### Example 417: N-[5-[4-[[(3S,4S)-4-hydroxy-1-methyl-pyrrolidin-3-yl]methoxy]-2-methyl-pyrazol-3-yl]pyrazolo[1,5-a]pyridin-2-yl]cyclopropanecarboxamide

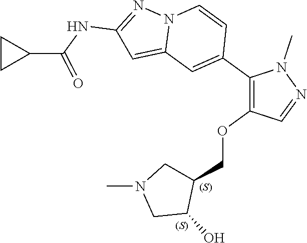

To a solution of N-(5-(4-(((3S,4S)-4-hydroxypyrrolidin-3-yl)methoxy)-1-methyl-1H-pyrazol-5-yl)pyrazolo[1,5-a]pyridin-2-yl)cyclopropanecarboxamide (120 mg, 0.303 mmol) (Example 416) in MeOH (3 mL) was added H(CHO)n (40.9 mg, 0.454 mmol) at room temperature. Then to the solution was added NaBH₃CN (38.0 mg, 0.605 mmol) and stirred for 1 h at room temperature. The mixture was purified by preparative HPLC with Waters Xbridge Prep OBD C18 150×40 mm×10 um, column (eluent: 0% to 31% (v/v) CH₃CN and H₂O with 10 mM NH₄HCO₃) to afford product. The product was suspended in water (10 mL), the mixture frozen using dry ice/EtOH, and then lyophilized to dryness to afford the product (49.3 mg, 39%) as a white solid. MS (ESI): mass calcd. for C₂₁H₂₆N₆O₃, 410.2. m/z found, 411.2 [M+H]⁺. ¹H NMR (400 MHz, DMSO-d6) δ 11.10 (s, 1H), 8.60 (d, J=7.2 Hz, 1H), 7.70 (d, J=0.8 Hz, 1H), 7.45 (s, 1H), 6.96 (dd, J=2.0, 7.2 Hz, 1H), 6.89 (s, 1H), 4.89 (br. s, 1H), 3.99-3.93 (m, 1H), 3.90-3.82 (m, 5H), 2.66-2.58 (m, 2H), 2.32-2.21 (m, 3H), 2.16 (s, 3H), 1.99-1.87 (m, 1H), 0.86-0.79 (m, 4H)

### Example 418: (3*R,4*R)—N-[5-[4-(1,4-dimethylpyrrolidin-3-yl)oxy-2-methyl-pyrazol-3-yl]pyrazolo[1,5-a]pyridin-2-yl]cyclopropanecarboxamide

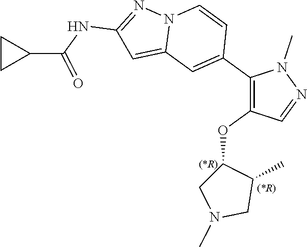

Step A. Tert-butyl (3S,4R)-3-methyl-4-(tosyloxy)pyrrolidine-1-carboxylate. The title compound (1.2 g, 72%) was prepared as described in Example 404 Step A, except (trans)-tert-butyl 3-hydroxy-4-methylpyrrolidine-1-carboxylate was used instead of (trans)-cyclohexane-1,4-diol.

Step B. (3R,4S)-tert-butyl 3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate. The title compound (440 mg, 54%) was prepared as described in Example 404 Step B, except tert-butyl (3S,4R)-3-methyl-4-(tosyloxy)pyrrolidine-1-carboxylate was used instead of (trans)-4-hydroxycyclohexyl 4-methylbenzenesulfonate.

Step C. (3*R,4*R)-tert-butyl 3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate. (3R,4S)-tert-butyl 3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate (440 mg, 0.916 mmol) was further purified by instrument Phenomenex-Cellulose-2 250×30 mm×5 μm column (eluent: 40% to 40% (v/v) 0.1% NH₃H₂O MEOH in supercritical CO₂. The pure fractions were collected and the volatiles were removed under reduced pressure. The product was suspended in water (300 mL), the mixture frozen using dry ice/EtOH, and then lyophilized to dryness to afford title compound (150 mg, 33%) as a yellow solid.

Step D. (3*R,4*R)—N-[5-[4-(1,4-dimethylpyrrolidin-3-yl)oxy-2-methyl-pyrazol-3-yl]pyrazolo[1,5-a]pyridin-2-yl]cyclopropanecarboxamide. The title compound (17.3 mg, 2 steps, 7%) was prepared as described in Example 36 Steps D-E, except (3*R,4*R)-tert-butyl 3-((5-(2-(cyclopropanecarboxamido)pyrazolo[1,5-a]pyridin-5-yl)-1-methyl-1H-pyrazol-4-yl)oxy)-4-methylpyrrolidine-1-carboxylate was used instead of tert-butyl (R)-2-(((5-(2-((5-((2-cyano-2-methylpropyl)carbamoyl)pyridin-2-yl)amino)pyrazolo[1,5-a]pyridin-5-yl)-1-methyl-1H-pyrazol-4-yl)oxy)methyl)azetidine-1-carboxylate in Step D and N-(5-(1-methyl-4-(((3*R,4*R)-4-methylpyrrolidin-3-yl)oxy)-1H-pyrazol-5-yl)pyrazolo[1,5-a]pyridin-2-yl)cyclopropanecarboxamide was used instead of (R)-6-((5-(4-(azetidin-2-ylmethoxy)-1-methyl-1H-pyrazol-5-yl)pyrazolo[1,5-a]pyridin-2-yl)amino)-N-(2-cyano-2-methylpropyl)nicotinamide in Step E. MS (ESI): mass calcd. for C₂₁H₂₆N₆O₂, 394.2. m/z found, 395.30 [M+H]⁺. ¹H NMR (400 MHz, CD3OD): 8.46 (d, J=7.6 Hz, 1H), 7.64 (d, J=1.2 Hz, 1H), 7.37 (s, 1H), 6.96 (dd, J=2.0, 7.6 Hz, 1H), 6.91 (s, 1H), 4.61-4.49 (m, 1H), 3.89 (s, 3H), 3.20-3.08 (m, 1H), 2.92-2.82 (m, 1H), 2.68-2.60 (m, 1H), 2.51-2.40 (m, 1H), 2.38-2.32 (m, 1H), 2.30 (s, 3H), 1.94-1.81 (m, 1H), 1.07 (d, J=7.2 Hz, 3H), 1.03-0.97 (m, 2H), 0.93-0.86 (m, 2H)

### Example 419: N-[5-[2-methyl-4-[[(2S)-1-methylpyrrolidin-2-yl]methoxy]pyrazol-3-yl]pyrazolo[1,5-a]pyridin-2-yl]cyclopropanecarboxamide

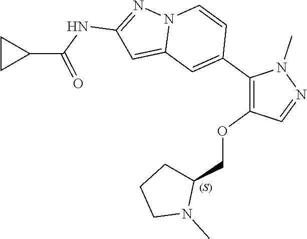

The title compound (59 mg, 45%) was prepared as described in Example 407, except (S)-(−)-1-Methyl-2-pyrrolidinemethanol was used instead of tert-butyl 4-hydroxypiperidine-1-carboxylate. MS (ESI): mass calcd. for C₂₁H₂₆N₆O₂, 394.2. m/z found, 395.2 [M+H]⁺. ¹H NMR (400 MHz, Methanol-d4) δ 8.35 (dt, J=7.2, 1.0 Hz, 1H), 7.55 (dd, J=2.0, 1.0 Hz, 1H), 7.34 (s, 1H), 6.86 (dd, J=7.2, 1.9 Hz, 1H), 6.80 (s, 1H), 3.93-3.81 (m, 2H), 3.78 (s, 3H), 2.92 (ddd, J=9.4, 5.7, 3.6 Hz, 1H), 2.56 (dq, J=8.1, 5.9, 5.0 Hz, 1H), 2.29 (s, 3H), 2.20 (td, J=9.5, 8.1 Hz, 1H), 1.97-1.81 (m, 1H), 1.77 (tt, J=8.1, 4.6 Hz, 1H), 1.66 (dddd, J=11.5, 9.7, 7.3, 5.4 Hz, 2H), 1.53 (dq, J=12.6, 7.2 Hz, 1H), 0.90 (dt, J=4.7, 3.1 Hz, 2H), 0.81 (dt, J=7.9, 3.2 Hz, 2H).

### Example 420: N-[5-[4-[[(2S)-1-ethylpyrrolidin-2-yl]methoxy]-2-methyl-pyrazol-3-yl]pyrazolo[1,5-a]pyridin-2-yl]cyclopropanecarboxamide

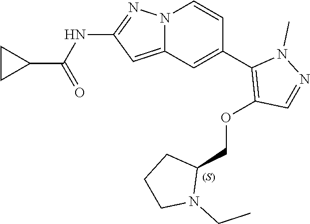

Step A. (S)—N-(5-(1-methyl-4-(pyrrolidin-2-ylmethoxy)-1H-pyrazol-5-yl)pyrazolo[1,5-a]pyridin-2-yl)cyclopropanecarboxamide. The title compound (99 mg, 99%) was prepared as described as Example 407, except boc-L-prolinol was used instead of tert-butyl 4-hydroxypiperidine-1-carboxylate.

Step B. N-[5-[4-[[(2S)-1-ethylpyrrolidin-2-yl]methoxy]-2-methyl-pyrazol-3-yl]pyrazolo[1,5-a]pyridin-2-yl]cyclopropanecarboxamide. The title compound (31 mg, 38%) was prepared as described in Example 8, except acetaldehyde was used instead of N-(5-(4-(azetidin-3-ylmethoxy)-1-methyl-1H-pyrazol-5-yl)pyrazolo[1,5-a]pyridin-2-yl)cyclopropanecarboxamide. MS (ESI): mass calcd. for C₂₂H₂₈N₆O₂, 408.2. m/z found, 409.0 [M+H]⁺. ¹H NMR (500 MHz, Methanol-d4) δ 8.44 (dt, J=7.2, 1.0 Hz, 1H), 7.63 (dd, J=2.0, 1.0 Hz, 1H), 7.42 (s, 1H), 6.94 (dd, J=7.2, 1.9 Hz, 1H), 6.90 (s, 1H), 3.94 (dd, J=5.7, 1.0 Hz, 2H), 3.87 (s, 3H), 3.11 (ddd, J=9.7, 6.5, 3.3 Hz, 1H), 2.97 (dq, J=12.1, 7.4 Hz, 1H), 2.87-2.78 (m, 1H), 2.33 (dq, J=12.0, 7.2 Hz, 1H), 2.25 (td, J=9.5, 7.4 Hz, 1H), 1.96 (dq, J=12.6, 8.4 Hz, 1H), 1.86 (tt, J=8.0, 4.5 Hz, 1H), 1.75 (dddd, J=14.6, 12.5, 5.7, 4.0 Hz, 2H), 1.62 (ddt, J=12.0, 8.5, 5.9 Hz, 1H), 1.03 (t, J=7.3 Hz, 3H), 0.99 (dt, J=4.5, 3.2 Hz, 2H), 0.90 (dt, J=8.0, 3.4 Hz, 2H).

### Example 421: (rac-trans)-N-[5-[4-(4-methoxypyrrolidin-3-yl)oxy-2-methyl-pyrazol-3-yl]pyrazolo[1,5-a]pyridin-2-yl]cyclopropanecarboxamide

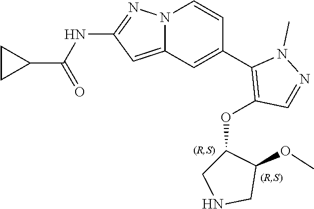

Step A. Tert-butyl (3R,4R)-3-((5-bromo-1-methyl-1H-pyrazol-4-yl)oxy)-4-hydroxypyrrolidine-1-carboxylate. The title compound (1.7 g, 95%) was prepared as described in Example 1 Step C, except (trans-rel)-tert-butyl 3-hydroxy-4-((1-methyl-1H-pyrazol-4-yl)oxy)pyrrolidine-1-carboxylate was used instead of tert-butyl (2S,4S)-2-methyl-4-((1-methyl-1H-pyrazol-4-yl)oxy)pyrrolidine-1-carboxylate.

Step B. (3RS,4RS)-tert-butyl 3-((5-bromo-1-methyl-1H-pyrazol-4-yl)oxy)-4-methoxypyrrolidine-1-carboxylate. The material NaH (133 mg, 60% in mineral oil, 3.31 mmol) was added in portions to a stirred mixture consisting of (3RS,4RS)-tert-butyl 3-((5-bromo-1-methyl-1H-pyrazol-4-yl)oxy)-4-methoxypyrrolidine-1-carboxylate (400 mg, 1.10 mmol), and THE (3 mL) at 0° C. and stirred room-temperature at 30 min. Then iodomethane (2.25 g, 15.9 mmol) was add to the reaction mixture and stirred overnight at room-temperature. The reaction mixture was concentrated to dryness under reduced pressure to give the crude product. The crude afford which was purified by FCC (petroleum ether:ethyl acetate=100:0 to 60:40) to afford pure product (113 mg, 27%) as a yellow oil.

Step C. (rac-trans)-N-[5-[4-(4-methoxypyrrolidin-3-yl)oxy-2-methyl-pyrazol-3-yl]pyrazolo[1,5-a]pyridin-2-yl]cyclopropanecarboxamide. The title compound (9.1 mg, 2 steps, 5%) was prepared as described in Example 5 Step D and Example 6 Step C, except (3RS,4RS)-tert-butyl 3-((5-bromo-1-methyl-1H-pyrazol-4-yl)oxy)-4-methoxypyrrolidine-1-carboxylate was used instead of rac-cis-tert-butyl (3s,4r)-3-((5-bromo-1-methyl-1H-pyrazol-4-yl)oxy)-4-methoxypyrrolidine-1-carboxylate in Example 5 and (trans-rel)-tert-butyl 3-((5-(2-(cyclopropanecarboxamido)pyrazolo[1,5-a]pyridin-5-yl)-1-methyl-1H-pyrazol-4-yl)oxy)-4-methoxypyrrolidine-1-carboxylate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate in Example 7. MS (ESI): mass calcd. for C₂₀H₂₄N₆O₃, 396.2. m/z found, 397.3 [M+H]⁺. ¹H NMR (400 MHz, DMSO-d6) δ 11.10 (s, 1H), 8.59 (d, J=6.8 Hz, 1H), 7.73-7.62 (m, 1H), 7.58-7.44 (m, 1H), 6.95 (dd, J=1.2, 7.2 Hz, 1H), 6.91 (s, 1H), 4.51-4.37 (m, 1H), 3.85 (s, 3H), 3.81-3.74 (m, 1H), 3.22 (s, 3H), 3.09-3.02 (m, 1H), 3.01-2.95 (m, 1H), 2.88-2.81 (m, 1H), 2.77-2.70 (m, 1H), 2.00-1.88 (m, 1H), 0.89-0.76 (m, 4H)

### Example 422: N-[5-[4-[[(3S,4S)-4-methoxypyrrolidin-3-yl]methoxy]-2-methyl-pyrazol-3-yl]pyrazolo[1,5-a]pyridin-2-yl]cyclopropanecarboxamide

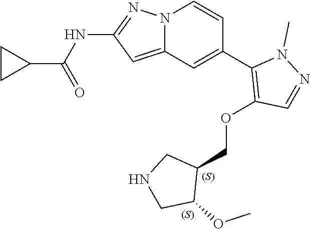

Step A. Tert-butyl (3 S,4S)-3-hydroxy-4-((tosyloxy)methyl)pyrrolidine-1-carboxylate. The title compound (1.3 g, 28%) was prepared as described in Example 404 Steps A-B, except (3S,4S)-tert-butyl 3-hydroxy-4-(hydroxymethyl)pyrrolidine-1-carboxylate was used instead of (trans)-cyclohexane-1,4-diol in Step A and (3S,4S)-tert-butyl 3-hydroxy-4-((tosyloxy)methyl)pyrrolidine-1-carboxylate was used instead of 1-methyl-1H-pyrazol-4-ol in Step B. In Step B 1-methyl-1H-pyrazol-4-ol was used instead of (trans)-4-hydroxycyclohexyl 4-methylbenzenesulfonate.

Step B. Tert-butyl (3S,4S)-3-methoxy-4-(((1-methyl-1H-pyrazol-4-yl)oxy)methyl)pyrrolidine-1-carboxylate. NaH (121 mg, 60% in mineral oil, 3.03 mmol) was added in portions to a stirred mixture consisting of (3R,4R)-tert-butyl 3-hydroxy-4-(((1-methyl-1H-pyrazol-4-yl)oxy)methyl)pyrrolidine-1-carboxylate (300 mg, 1.01 mmol), and THE (3 mL) at 0° C. and stirred room-temperature for 30 min.

Then iodomethane (1.69 g, 11.9 mmol) was add to the reaction mixture and stirred overnight at room-temperature. The mixture was added aq. NH₄Cl (10 mL) at 0° C. and stirred for 0.5 h. The reaction mixture was concentrated to dryness under reduced pressure to give the crude product. The crude afford which was purified by FCC (petroleum ether:ethyl acetate-=1:0 to 1:1) to afford product (200 mg, 64%) as a yellow oil.

Step C. (3S,4S)-tert-butyl 3-(((5-(2-(cyclopropanecarboxamido)pyrazolo[1,5-a]pyridin-5-yl)-1-methyl-1H-pyrazol-4-yl)oxy)methyl)-4-methoxypyrrolidine-1-carboxylate. The title compound (230 mg, 2 steps, 88%) was prepared as described in Example 1 Steps C-D, except (3S,4S)-tert-butyl 3-methoxy-4-(((1-methyl-1H-pyrazol-4-yl)oxy)methyl)pyrrolidine-1-carboxylate was used instead of tert-butyl (2S,4S)-2-methyl-4-((1-methyl-1H-pyrazol-4-yl)oxy)pyrrolidine-1-carboxylate in Step C and (3S,4S)-tert-butyl 3-(((5-bromo-1-methyl-1H-pyrazol-4-yl)oxy)methyl)-4-methoxypyrrolidine-1-carboxylate was used instead of tert-butyl (2S,4S)-4-((5-bromo-1-methyl-1H-pyrazol-4-yl)oxy)-2-methylpyrrolidine-1-carboxylate in Step D.

Step D. N-[5-[4-[[(3S,4S)-4-methoxypyrrolidin-3-yl]methoxy]-2-methyl-pyrazol-3-yl]pyrazolo[1,5-a]pyridin-2-yl]cyclopropanecarboxamide. A solution consisting of (3S,4S)-tert-butyl 3-(((5-(2-(cyclopropanecarboxamido)pyrazolo[1,5-a]pyridin-5-yl)-1-methyl-1H-pyrazol-4-yl)oxy)methyl)-4-methoxypyrrolidine-1-carboxylate (110 mg, 0.215 mmol), HCl/1,4-dioxane (3 mL, 4 M) was stirred at room temperature for 1 h. The mixture was concentrated under reduced pressure. The reaction mixture was poured into 10 mL of H2O. The mixture was neutralized with aq. NaHCO₃ and concentrated under reduced pressure. The mixture was purified by preparative HPLC with Waters Xbridge Prep OBD C18 150×40 mm×10 um, column (eluent: 0% to 45% (v/v) CH₃CN and H₂O with 10 mM NH₄HCO₃) to afford product. The product was purified by SFC with DAICEL CHIRALCEL OD-H (250 mm×30 mm, 5 um), column (eluent: 40% to 40% (v/v) EtOH with 0.1% NH₃·H₂O in supercritical CO₂) to afford pure product. The product was suspended in water (10 mL), the mixture frozen using dry ice/EtOH, and then lyophilized to dryness to afford product (17.5 mg, 19%) as a white solid. MS (ESI): mass calcd. for C₂₁H₂₆N₆O₃, 410.2. m/z found, 411.2 [M+H]⁺. ¹H NMR (400 MHz, DMSO-d6) δ 11.10 (s, 1H), 8.61 (d, J=6.8 Hz, 1H), 7.69 (s, 1H), 7.46 (s, 1H), 6.96 (dd, J=1.6, 6.8 Hz, 1H), 6.89 (s, 1H), 3.92-3.83 (m, 5H), 3.68-3.58 (m, 1H), 3.28-3.07 (m, 4H), 3.01-2.93 (m, 1H), 2.92-2.83 (m, 1H), 2.78-2.68 (m, 1H), 2.36-2.25 (m, 1H), 1.99-1.89 (m, 1H), 0.88-0.77 (m, 4H)

### Example 423: N-[5-[4-[[(3S,4S)-4-methoxy-1-methyl-pyrrolidin-3-yl]methoxy]-2-methyl-pyrazol-3-yl]pyrazolo[1,5-a]pyridin-2-yl]cyclopropanecarboxamide

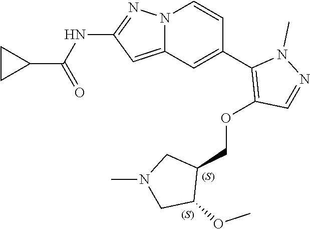

To a solution of N-(5-(4-(((3S,4S)-4-fluoropyrrolidin-3-yl)oxy)-1-methyl-1H-pyrazol-5-yl)pyrazolo[1,5-a]pyridin-2-yl)cyclopropanecarboxamide (Example 417) (100 mg, 0.244 mmol) in MeOH (3 mL) was added H(CHO)n (32.9 mg, 0.365 mmol) at room temperature. Then to the solution was added NaBH₃CN (30.6 mg, 0.487 mmol) and stirred for 1 h at room temperature. The mixture was purified by preparative HPLC with Boston Prime C18 150×30 mm×5 um, column (eluent: 25% to 55% (v/v) CH₃CN and H₂O with 0.04% NH₃H₂O+10 mM NH₄HCO₃) to afford product. The product was suspended in water (10 mL), the mixture frozen using dry ice/EtOH, and then lyophilized to dryness to afford P1 (19.1 mg, 18%) as an off-white solid. MS (ESI): mass calcd. for C₂₂H₂₈N₆O₃, 424.2. m/z found, 425.3 [M+H]⁺. ¹H NMR (400 MHz, DMSO-d6) δ 11.10 (s, 1H), 8.60 (d, J=7.6 Hz, 1H), 7.69 (d, J=1.2 Hz, 1H), 7.46 (s, 1H), 6.96 (dd, J=2.0, 7.2 Hz, 1H), 6.88 (s, 1H), 3.95-3.87 (m, 2H), 3.86 (s, 3H), 3.65-3.60 (m, 1H), 3.13 (s, 3H), 2.69-2.60 (m, 2H), 2.46-2.40 (m, 1H), 2.38-2.30 (m, 1H), 2.23-2.19 (m, 1H), 2.18 (s, 3H), 1.98-1.87 (m, 1H), 0.87-0.79 (m, 4H)

### Example 424: N-[5-[4-[[(3R,4R)-4-hydroxy-1-methyl-pyrrolidin-3-yl]methoxy]-2-methyl-pyrazol-3-yl]pyrazolo[1,5-a]pyridin-2-yl]cyclopropanecarboxamide

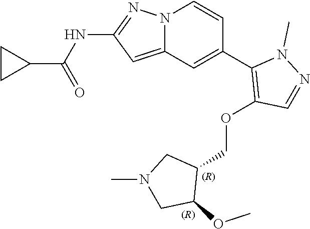

The title compound (50 mg, 4 steps, 4%) was prepared as described in Example 1 steps A-B, E-F, except (3R,4R)-tert-butyl 3-hydroxy-4-(hydroxymethyl)pyrrolidine-1-carboxylate was used instead of (2s,4r)-tert-butyl 4-hydroxy-2-methylpyrrolidine-1-carboxylate in Step A, (3R,4R)-tert-butyl 3-hydroxy-4-((tosyloxy)methyl)pyrrolidine-1-carboxylate was used instead of (2S,4R)-2-methyl-4-(tosyloxy)pyrrolidine-1-carboxylate in Step B and Intermediate 4 was used instead of 1-methyl-1h-pyrazol-4-ol in Step B, (3R,4R)-tert-butyl 3-(((5-(2-(cyclopropanecarboxamido)pyrazolo[1,5-a]pyridin-5-yl)-1-methyl-1H-pyrazol-4-yl)oxy)methyl)-4-hydroxypyrrolidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E, and N-(5-(4-(((3R,4R)-4-hydroxypyrrolidin-3-yl)methoxy)-1-methyl-1H-pyrazol-5-yl)pyrazolo[1,5-a]pyridin-2-yl)cyclopropanecarboxamide was used instead of containing N-(5-(I-methyl-4-(((3S,5S)-5-methylpyrrolidin-3-yl)oxy)-1H-pyrazol-5-yl)pyrazolo[1,5-a]pyridin-2-yl)cyclopropanecarboxamide in Step F. MS (ESI): mass calcd. for C₂₁H₂₆N₆O₃, 410.2. m/z found, 411.2 [M+H]⁺. ¹H NMR (400 MHz, DMSO-d6) δ 11.10 (s, 1H), 8.59 (d, J=7.2 Hz, 1H), 7.70 (s, 1H), 7.46 (s, 1H), 6.96 (dd, J=0.8, 6.8 Hz, 1H), 6.89 (s, 1H), 4.97 (d, J=4.4 Hz, 1H), 3.98-3.83 (m, 6H), 2.75-2.64 (m, 2H), 2.40-2.36 (m, 1H), 2.33-2.21 (m, 5H), 1.97-1.89 (m, 1H), 0.85-0.79 (m, 4H)

### Example 425: N-[5-[4-[[(2R)-1-(cyanomethyl)azetidin-2-yl]methoxy]-2-methyl-pyrazol-3-yl]pyrazolo[1,5-a]pyridin-2-yl]cyclopropanecarboxamide

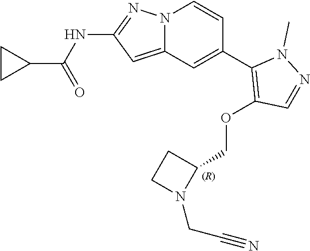

The title compound (75 mg, 15%) was prepared as described in Example 38, except N-[5-[4-[[(2R)-1-(cyanomethyl)azetidin-2-yl]methoxy]-2-methyl-pyrazol-3-yl]pyrazolo[1,5-a]pyridin-2-yl]cyclopropanecarboxamide was isolated instead of N-[5-[2-methyl-4-[[(2R)-1-methylazetidin-2-yl]methoxy]pyrazol-3-yl]pyrazolo[1,5-a]pyridin-2-yl]cyclopropanecarboxamide. MS (ESI): mass calcd. for C₂₁H₂₃N₇O₂, 405.2. m/z found, 406.20 [M+H]⁺. ¹H NMR (500 MHz, Chloroform-d) δ 9.06 (s, 1H), 8.35 (d, J=7.2 Hz, 1H), 7.48 (dd, J=1.9, 0.9 Hz, 1H), 7.34 (s, 1H), 7.04 (s, 1H), 6.85 (dd, J=7.2, 1.9 Hz, 1H), 4.04-3.95 (m, 2H), 3.91 (s, 3H), 3.81 (qd, J=8.1, 5.5 Hz, 1H), 3.54-3.46 (m, 1H), 3.41 (d, J=17.5 Hz, 1H), 3.37 (td, J=7.4, 3.1 Hz, 1H), 3.26 (td, J=8.7, 6.8 Hz, 1H), 2.13-1.99 (m, 2H), 1.62 (dq, J=8.7, 4.3 Hz, 1H), 1.25-1.12 (m, 2H), 0.90 (dq, J=7.3, 4.1 Hz, 2H).

### Example 426: N-[5-[4-[(2R)-2-amino-3-hydroxy-3-methyl-butoxy]-2-methyl-pyrazol-3-yl]pyrazolo[1,5-a]pyridin-2-yl]cyclopropanecarboxamide

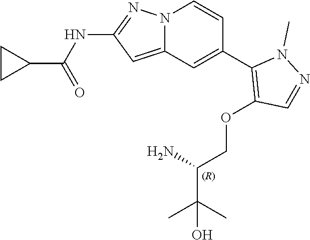

Step A. Tert-butyl (S)-5-(((4-bromo-5-methylisoxazol-3-yl)oxy)methyl)-2,2-dimethylmorpholine-4-carboxylate. The title compound (540 mg, 47%) was prepared as described in Example 389, except PPh₃ was used instead of TPP in Step A.

Step B. Tert-butyl (S)-5-(((4-(2-(cyclopropanecarboxamido)pyrazolo[1,5-a]pyridin-5-yl)-5-methylisoxazol-3-yl)oxy)methyl)-2,2-dimethylmorpholine-4-carboxylate. The title compound (500 mg, 77%) was prepared as described in Step D of Example 1, except tert-butyl (S)-5-(((4-bromo-5-methylisoxazol-3-yl)oxy)methyl)-2,2-dimethylmorpholine-4-carboxylate was used instead of tert-butyl (2S,4S)-4-((5-bromo-1-methyl-1H-pyrazol-4-yl)oxy)-2-methylpyrrolidine-1-carboxylate.

Step C. N-[5-[4-[(2R)-2-amino-3-hydroxy-3-methyl-butoxy]-2-methyl-pyrazol-3-yl]pyrazolo[1,5-a]pyridin-2-yl]cyclopropanecarboxamide. The title compound (67.7 mg, 90%) was prepared as described in Example 6 Step C, except tert-butyl (S)-5-(((4-(2-(cyclopropanecarboxamido)pyrazolo[1,5-a]pyridin-5-yl)-5-methylisoxazol-3-yl)oxy)methyl)-2,2-dimethylmorpholine-4-carboxylate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate. MS (ESI): mass calcd. for C₂₀H₂₆N₆O₃, 398.2. m/z found, 399.1 [M+H]⁺. ¹H NMR (400 MHz, DMSO-d6) δ 11.12 (s, 1H), 8.58 (d, J=7.2 Hz, 1H), 8.13 (s, 3H), 7.80 (s, 1H), 7.55 (s, 1H), 7.07-7.01 (m, 1H), 6.91 (s, 1H), 4.21-4.19 (m, 1H), 4.04-4.02 (m, 1H), 3.87 (s, 3H), 3.31-3.23 (m, 1H), 2.00-1.91 (m, 1H), 1.23 (s, 3H), 1.16 (s, 3H), 0.86-0.79 (m, 4H)

### Example 427: N-[5-[4-[[(2R)-1-ethylpyrrolidin-2-yl]methoxy]-2-methyl-pyrazol-3-yl]pyrazolo[1,5-a]pyridin-2-yl]cyclopropanecarboxamide

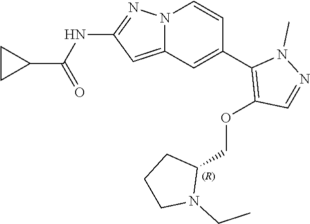

The title compound (24.5 mg, 4 steps, 13%) was prepared as described in Example 5 Steps A-C then Example 26 Step A, except (R)-tert-butyl 2-(hydroxymethyl)pyrrolidine-1-carboxylate was used instead of trans-rac-tert-butyl (3r,4r)-3-hydroxy-4-methoxypyrrolidine-1-carboxylate in Example 6 Step A, (R)-tert-butyl 2-((tosyloxy)methyl)pyrrolidine-1-carboxylate was used instead of rac-trans-tert-butyl (3r,4r)-3-methoxy-4-(tosyloxy)pyrrolidine-1-carboxylate in Example 6 Step B, and (R)-tert-butyl 2-(((1-methyl-1H-pyrazol-4-yl)oxy)methyl)pyrrolidine-1-carboxylate was used instead of rac-tert-butyl (3r,4s)-3-methoxy-4-((1-methyl-1H-pyrazol-4-yl)oxy)pyrrolidine-1-carboxylate in Example 6 Step C. In Example 27 Step A, (R)-tert-butyl 2-(((5-bromo-1-methyl-1H-pyrazol-4-yl)oxy)methyl)pyrrolidine-1-carboxylate was used instead of tert-butyl (R)-2-(((5-bromo-1-methyl-1H-pyrazol-4-yl)oxy)methyl)azetidine-1-carboxylate. MS (ESI): mass calcd. for C₂₂H₂₈N₆O₂, 408.2. m/z found, 409.2 [M+H]⁺. ¹H NMR (400 MHz, DMSO-d6) δ 11.10 (s, 1H), 8.60 (d, J=7.2 Hz, 1H), 7.69 (s, 1H), 7.45 (s, 1H), 6.95 (dd, J=1.6, 7.2 Hz, 1H), 6.88 (s, 1H), 3.93-3.87 (m, 1H), 3.85 (s, 3H), 3.79-3.72 (m, 1H), 3.06-2.96 (m, 1H), 2.90-2.77 (m, 1H), 2.72-2.65 (m, 1H), 2.30-2.21 (m, 1H), 2.19-2.06 (m, 1H), 1.97-1.88 (m, 1H), 1.87-1.76 (m, 1H), 1.69-1.59 (m, 2H), 1.58-1.48 (m, 1H), 0.95 (t, J=7.2 Hz, 3H), 0.86-0.79 (m, 4H)

### Example 428: N-[5-[4-[[(2R)-1-(2-methoxyethyl)pyrrolidin-2-yl]methoxy]-2-methyl-pyrazol-3-yl]pyrazolo[1,5-a]pyridin-2-yl]cyclopropanecarboxamide

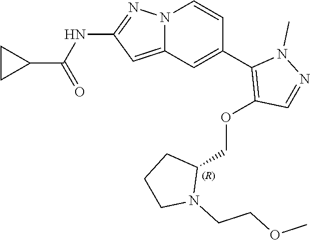

Step A. (R)—N-(5-(1-methyl-4-(pyrrolidin-2-ylmethoxy)-1H-pyrazol-5-yl)pyrazolo[1,5-a]pyridin-2-yl)cyclopropanecarboxamide. The title compound (2 g, 3 steps, 12%) was prepared as described in Example 5 Steps A-C and, except (R)-tert-butyl 2-(hydroxymethyl)pyrrolidine-1-carboxylate was used instead of trans-rac-tert-butyl (3r,4r)-3-hydroxy-4-methoxypyrrolidine-1-carboxylate in Example 6 Step A, (R)-tert-butyl 2-((tosyloxy)methyl)pyrrolidine-1-carboxylate was used instead of rac-trans-tert-butyl (3r,4r)-3-methoxy-4-(tosyloxy)pyrrolidine-1-carboxylate in Example 6 Step B, and (R)-tert-butyl 2-(((1-methyl-1H-pyrazol-4-yl)oxy)methyl)pyrrolidine-1-carboxylate was used instead of rac-tert-butyl (3r,4s)-3-methoxy-4-((1-methyl-1H-pyrazol-4-yl)oxy)pyrrolidine-1-carboxylate in Example 6 Step C. In Example 27 Step A, (R)-tert-butyl 2-(((5-bromo-1-methyl-1H-pyrazol-4-yl)oxy)methyl)pyrrolidine-1-carboxylate was used instead of tert-butyl (R)-2-(((5-bromo-1-methyl-1H-pyrazol-4-yl)oxy)methyl)azetidine-1-carboxylate.

Step B. N-[5-[4-[[(2R)-1-(2-methoxyethyl)pyrrolidin-2-yl]methoxy]-2-methyl-pyrazol-3-yl]pyrazolo[1,5-a]pyridin-2-yl]cyclopropanecarboxamide. The title compound (38.8 mg, 22%) was prepared as follows. A mixture consisting of (R)—N-(5-(1-methyl-4-(pyrrolidin-2-ylmethoxy)-1H-pyrazol-5-yl)pyrazolo[1,5-a]pyridin-2-yl)cyclopropanecarboxamide (150 mg, 0.394 mmol), 1-bromo-2-methoxyethane (65.8 mg, 0.473 mmol), DIEA (153 mg, 1.18 mmol) and DMF (3 mL) was stirred at room temperature overnight. The mixture was purified by preparative HPLC with Boston Prime C18 150×30 mm×5 um, column (eluent: 30% to 60% (v/v) CH₃CN and H₂O with 0.04% NH₃H₂O+10 mM NH₄HCO₃) to afford product. The product was suspended in water (10 mL), the mixture frozen using dry ice/EtOH, and then lyophilized to dryness to afford the product as a white solid. MS (ESI): mass calcd. for C₂₃H₃₀N₆O₃, 438.2. m/z found, 439.3 [M+H]⁺. ¹H NMR (400 MHz, DMSO-d6) δ 11.09 (s, 1H), 8.58 (d, J=7.2 Hz, 1H), 7.69 (s, 1H), 7.45 (s, 1H), 6.96 (dd, J=1.6, 7.2 Hz, 1H), 6.89 (s, 1H), 3.93-3.82 (m, 4H), 3.80-3.73 (m, 1H), 3.34-3.29 (m, 2H), 3.15 (s, 3H), 3.04-2.92 (m, 2H), 2.81-2.72 (m, 1H), 2.46-2.39 (m, 1H), 2.27-2.16 (m, 1H), 2.00-1.88 (m, 1H), 1.87-1.76 (m, 1H), 1.70-1.57 (m, 2H), 1.56-1.47 (m, 1H), 0.90-0.77 (m, 4H)

### Example 429: N-[5-[4-[[(2R)-1-benzylazetidin-2-yl]methoxy]-2-methyl-pyrazol-3-yl]pyrazolo[1,5-a]pyridin-2-yl]cyclopropanecarboxamide

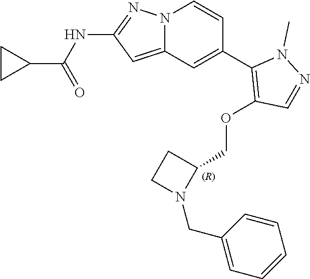

A mixture consisting of (R)—N-(5-(4-(azetidin-2-ylmethoxy)-1-methyl-1H-pyrazol-5-yl)pyrazolo[1,5-a]pyridin-2-yl)cyclopropanecarboxamide (Example 346, 90 mg, 0.187 mmol), benzaldehyde (46.9 mg, 0.442 mmol) and MeOH (4 mL) was stirred at 25° C. for 5 min. Then NaBH₃CN (34.0 mg, 0.541 mmol) was added to the reaction mixture. The reaction mixture was stirred at 25° C. for 12 hours. The mixture was filtered and the filtrate was purified by preparative HPLC using a Boston Prime C18 150×30 mm×5 um (eluent: 40% to 70% (v/v) CH₃CN and H₂O with 0.04% NH₃) to afford pure product. The pure fractions were collected and the volatiles were removed under reduced pressure. The product was suspended in water (10 mL), the mixture frozen using dry ice/ethanol, and then lyophilized to dryness to afford the product (18.3 mg, 21.4%) as a white solid. MS (ESI): mass calcd. for C₂₆H₂₈N₆O₂, 456.2. m/z found, 457.2 [M+H]⁺. ¹H NMR (400 MHz, CDCl₃) δ 8.47-8.39 (m, 1H), 8.22 (d, J=7.2 Hz, 1H), 7.45 (s, 1H), 7.25-7.18 (m, 5H), 6.98 (s, 1H), 6.82 (dd, J=2.0, 7.2 Hz, 1H), 3.89 (s, 3H), 3.83 (d, J=5.6 Hz, 2H), 3.79-3.70 (m, 1H), 3.57-3.47 (m, 2H), 3.35-3.29 (m, 1H), 2.93-2.85 (m, 1H), 2.07-1.96 (m, 2H), 1.61-1.50 (m, 1H), 1.17-1.11 (m, 2H), 0.94-0.87 (m, 2H)

### Example 430: N-[5-[2-methyl-4-[[(2R)-1-[(1-methylpyrazol-3-yl)methyl]azetidin-2-yl]methoxy]pyrazol-3-yl]pyrazolo[1,5-a]pyridin-2-yl]cyclopropanecarboxamide

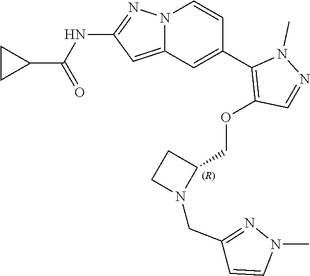

The title compound (21.3 mg, 26.6%) was prepared as described in Example 429, except 3-(chloromethyl)-1-methyl-1H-pyrazole was used instead of benzaldehyde, TEA was used instead of NaBH₃CN, and DMF (4 mL) was used instead of MeOH. MS (ESI): mass calcd. for C₂₄H₂₈N₈O₂, 460.2. m/z found, 461.2 [M+H]⁺. ¹H NMR (400 MHz, CDCl₃) δ 8.47-8.39 (m, 1H), 8.28 (d, J=7.6 Hz, 1H), 7.47 (s, 1H), 7.29 (s, 1H), 7.24 (d, J=2.0 Hz, 1H), 6.97 (s, 1H), 6.88 (dd, J=1.6, 7.2 Hz, 1H), 6.10 (d, J=2.0 Hz, 1H), 3.95-3.86 (m, 5H), 3.84 (s, 3H), 3.80-3.72 (m, 1H), 3.60-3.50 (m, 2H), 3.40-3.33 (m, 1H), 3.01-2.92 (m, 1H), 2.07-1.97 (m, 2H), 1.61-1.52 (m, 1H), 1.18-1.10 (m, 2H), 0.94-0.86 (m, 2H)

### Example 431: N-[5-[2-methyl-4-[[(2R)-1-[2-(trifluoromethoxy)ethyl]azetidin-2-yl]methoxy]pyrazol-3-yl]pyrazolo[1,5-a]pyridin-2-yl]cyclopropanecarboxamide

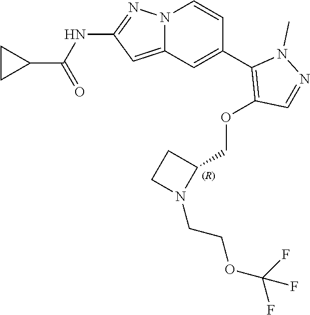

The title compound (21.3 mg, 26.6%) was prepared as described in Example 429, except 3-(chloromethyl)-1-methyl-1H-pyrazole was used instead of benzaldehyde, TEA was used instead of NaBH₃CN, and DMF (4 mL) was used instead of MeOH. MS (ESI): mass calcd. for C₂₂H₂₅F₃N₆O₃, 478.2. m/z found, 479.1 [M+H]⁺. ¹H NMR (400 MHz, DMSO-d6) δ 11.10 (s, 1H), 8.57 (d, J=7.2 Hz, 1H), 7.67 (d, J=0.8 Hz, 1H), 7.44 (s, 1H), 6.94 (dd, J=1.6, 6.8 Hz, 1H), 6.88 (s, 1H), 3.98-3.89 (m, 4H), 3.85 (s, 3H), 3.50-3.40 (m, 1H), 3.32-3.25 (m, 1H), 2.91-2.76 (m, 2H), 2.64-2.55 (m, 1H), 2.03-1.81 (m, 3H), 0.87-0.76 (m, 4H) 19F NMR (376 MHz, DMSO-d6) δ −59.00 (s, 3F)

### Example 432: N-[5-[2-methyl-4-[[(1S,4R,5R)-3-methyl-3-azabicyclo[3.1.0]hexan-4-yl]methoxy]pyrazol-3-yl]pyrazolo[1,5-a]pyridin-2-yl]cyclopropanecarboxamide

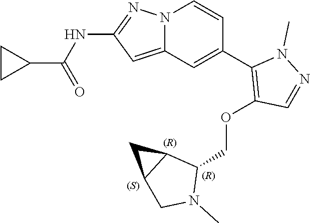

Step A. Tert-butyl (1R,2R,5S)-2-(hydroxymethyl)-3-azabicyclo[3.1.0]hexane-3-carboxylate. The title compound (400 mg, 107%, impure) was prepared as follows. To a mixture consisting of (1R,2R,5S)-3-(tert-butoxycarbonyl)-3-azabicyclo[3.1.0]hexane-2-carboxylic acid (400 mg, 1.76 mmol) and THE (20 mL) was added BH₃-THF (8.8 mL, 8.8 mmol, 1 M) at 0° C. The reaction mixture was stirred at room-temperature for 16 h. The resultant mixture was added dropwise to MeOH (50 mL) at 0° C., and then the mixture was stirred for 1 h at room temperature. The reaction mixture dryness under reduced pressure to give the title compound (400 mg, crude) as a colorless oil.

Step B. N-[5-[2-methyl-4-[[(1S,4R,5R)-3-methyl-3-azabicyclo[3.1.0]hexan-4-yl]methoxy]pyrazol-3-yl]pyrazolo[1,5-a]pyridin-2-yl]cyclopropanecarboxamide. The title compound (17.9 mg, 4 steps, 1%) was prepared as described in Example 9 Steps A-C and Example 17 Step C, except tert-butyl (1R,2R,5S)-2-(hydroxymethyl)-3-azabicyclo[3.1.0]hexane-3-carboxylate was used instead of tert-butyl (S)-2-(hydroxymethyl)azetidine-1-carboxylate in Example 10 Step A, (1R,2R,5S)-tert-butyl 2-((tosyloxy)methyl)-3-azabicyclo[3.1.0]hexane-3-carboxylate was used instead of tert-butyl (S)-2-((tosyloxy)methyl)azetidine-1-carboxylate in Example 10 Step B, HCl/1,4-dioxane (5 mL) was used instead of TFA/DCM in Example 10 Step C, and N-(5-(4-((1R,2R,5S)-3-azabicyclo[3.1.0]hexan-2-ylmethoxy)-1-methyl-1H-pyrazol-5-yl)pyrazolo[1,5-a]pyridin-2-yl)cyclopropanecarboxamide hydrochloride was used instead of (R)-5-(4-(azetidin-2-ylmethoxy)-1-methyl-1H-pyrazol-5-yl)-N-(5,6-dimethylpyridazin-3-yl)pyrazolo[1,5-a]pyridin-2-amine in Example 18 Step C. MS (ESI): mass calcd. for C₂₂H₂₆N₆O₂, 406.2. m/z found, 407.1 [M+H]⁺. ¹H NMR (400 MHz, DMSO-d6) δ 11.12 (s, 1H), 8.62 (d, J=7.6 Hz, 1H), 7.75-7.64 (m, 1H), 7.48 (s, 1H), 6.99 (dd, J=2.0, 7.2 Hz, 1H), 6.88 (s, 1H), 3.99-3.91 (m, 2H), 3.87 (s, 3H), 3.08-2.98 (m, 1H), 2.80-2.72 (m, 1H), 2.62-2.58 (m, 1H), 2.28 (s, 3H), 1.98-1.88 (m, 1H), 1.44-1.32 (m, 2H), 0.90-0.76 (m, 4H), 0.53-0.45 (m, 1H), 0.44-0.37 (m, 1H)

### Example 433: N-[5-[2-methyl-4-[[(2R)-1-(oxetan-3-ylmethyl)azetidin-2-yl]methoxy]pyrazol-3-yl]pyrazolo[1,5-a]pyridin-2-yl]cyclopropanecarboxamide

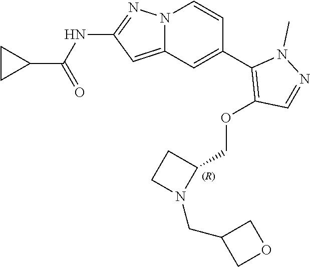

The title compound (70.6 mg, 39%) was prepared as described in Example 429, except oxetane-3-carbaldehyde was used instead of benzaldehyde. MS (ESI): mass calcd. for C₂₃H₂₈N₆O₃, 436.2. m/z found, 437.1 [M+H]⁺. ¹H NMR (400 MHz, CDCl₃) δ 8.35-8.29 (m, 2H), 7.49 (d, J=0.8 Hz, 1H), 7.33 (s, 1H), 7.00 (s, 1H), 6.85 (dd, J=2.0, 7.2 Hz, 1H), 4.68-4.59 (m, 2H), 4.29-4.21 (m, 2H), 3.95-3.91 (m, 2H), 3.90 (s, 3H), 3.47-3.38 (m, 1H), 3.37-3.30 (m, 1H), 2.99-2.77 (m, 3H), 2.69-2.61 (m, 1H), 2.08-1.90 (m, 2H), 1.61,−1.53 (m, 1H), 1.18-1.11 (m, 2H), 0.95-0.87 (m, 2H)

### Example 434: N-[5-[2-methyl-4-[[1-(methylamino)cyclopropyl]methoxy]pyrazol-3-yl]pyrazolo[1,5-a]pyridin-2-yl]cyclopropanecarboxamide

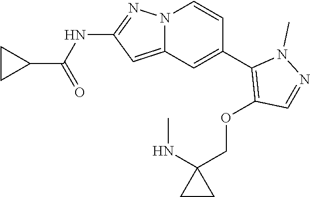 [SMILES: CNC1(COc2cnn(C)c2-c2ccn3nc(NC(=O)C4CC4)cc3c2)CC1]

Step A. Tert-butyl (1-(((1-methyl-1H-pyrazol-4-yl)oxy)methyl)cyclopropyl)carbamate. DIAD (1.5 g, 7.6 mmol) was added to a solution consisting of 1-methyl-1H-pyrazol-4-ol (500 mg, 5.10 mmol), tert-butyl (1-(hydroxymethyl)cyclopropyl)carbamate (1.05 g, 5.61 mmol), PPh₃ (2.7 g, 10 mmol), and toluene (10 mL). The resultant mixture was stirred at 110° C. for overnight. The reaction mixture was directly concentrated to afford the crude product, which was purified by FCC (eluent: petroleum ether:ethyl acetate=0 to 50%) to afford the title compound (2.4 g, 32%) as a yellow solid.

Step B. Tert-butyl methyl(1-(((1-methyl-1H-pyrazol-4-yl)oxy)methyl)cyclopropyl)carbamate. NaH (229.8 mg, 5.746 mmol, 60.0% purity) was added to a suspension consisting of tert-butyl (1-(((1-methyl-1H-pyrazol-4-yl)oxy)methyl)cyclopropyl)carbamate (2.4 g, 2.9 mmol) and DMF (10 mL). The resultant mixture was stirred for 10 minutes and then Mel (510 mg, 3.59 mmol) was added to the reaction mixture, the resultant reaction mixture was stirred at 0° C. for 2 h. Additional Mel (510 mg, 3.59 mmol) was added and the resultant mixture was stirred for another 2 h at 0° C. The reaction was quenched with NH₄Cl (20 mL), and directly concentrated to afford the crude product (2.4 g, crude), which directly used to next step reaction without further purify.

Step C. Tert-butyl (1-(((5-bromo-1-methyl-1H-pyrazol-4-yl)oxy)methyl)cyclopropyl)(methyl)carbamate. A mixture consisting of tert-butyl methyl(1-(((1-methyl-1H-pyrazol-4-yl)oxy)methyl)cyclopropyl)carbamate (180 mg, 0.640 mmol), NBS (114 mg, 0.640 mmol), ACN (5 mL) was stirred for 2 h. The reaction mixture was directly concentrated to afford the crude product, which was directly used to next step reaction without further purification.

Step D. tert-butyl (1-(((5-(2-(cyclopropanecarboxamido)pyrazolo[1,5-a]pyridin-5-yl)-1-methyl-1H-pyrazol-4-yl)oxy)methyl)cyclopropyl)(methyl)carbamate. A mixture consisting of N-(5-(4,4,5,5-tetramethyl-1,3,2-dioxaborolan-2-yl)pyrazolo[1,5-a]pyridin-2-yl)cyclopropanecarboxamide (218 mg, 0.666 mmol), tert-butyl (1-(((5-bromo-1-methyl-1H-pyrazol-4-yl)oxy)methyl)cyclopropyl)(methyl)carbamate (200 mg, 0.555 mmol), Pd(dppf)Cl₂ (18 mg, 0.028 mmol), K₃PO₄ (354 mg, 1.67 mmol), and 1,4-dioxane/H₂O (5 mL) was stirred for 2 h at 90° C. under Ar. The reaction mixture was directly concentrated to afford the crude product, which was purified by FCC (eluent: petroleum ether:ethyl acetate=0% to 70%) to afford the title compound (220 mg, 78%) as a yellow solid.

Step E. N-[5-[2-methyl-4-[[1-(methylamino)cyclopropyl]methoxy]pyrazol-3-yl]pyrazolo[1,5-a]pyridin-2-yl]cyclopropanecarboxamide. A mixture consisting of tert-butyl (1-(((5-(2-(cyclopropanecarboxamido)pyrazolo[1,5-a]pyridin-5-yl)-1-methyl-1H-pyrazol-4-yl)oxy)methyl)cyclopropyl)(methyl)carbamate (220 mg, 0.458 mmol), and HCl/1,4-dixoane (5 mL) was stirred for 2 h at room temperature. The reaction mixture was directly concentrated to afford the crude product. Half of the product was directly used for the next reaction without further purified, the another product was purified by preparative HPLC using a Boston Prime C18 150×30 mm×5 μm column (eluent: 20% to 50% (v/v) CH₃CN and H₂O with 10 mM NH₄HCO₃) to afford pure product. The product was suspended in water (10 mL), the mixture frozen using dry ice/acetone, and then lyophilized to dryness to afford the still impure title compound (47.5 mg, 27%) as a yellow solid. MS (ESI): mass calcd. for C₂₀H₂₄N₆O₂, 380.2. m/z found, 381.1 [M+H]⁺. ¹H NMR (400 MHz, DMSO-d6) δ 11.11 (s, 1H), 8.60 (d, J=7.2 Hz, 1H), 7.75-7.71 (m, 1H), 7.45 (s, 1H), 7.00 (dd, J=1.6, 7.2 Hz, 1H), 6.90 (s, 1H), 3.91 (s, 2H), 3.86 (s, 3H), 2.28 (s, 3H), 1.97-1.87 (m, 1H), 0.89-0.77 (m, 4H), 0.62-0.57 (m, 2H), 0.56-0.51 (m, 2H)

### Example 435: N-[5-[4-[[(2R)-1-(2-methoxy-2-methyl-propyl)azetidin-2-yl]methoxy]-2-methyl-pyrazol-3-yl]pyrazolo[1,5-a]pyridin-2-yl]cyclopropanecarboxamide

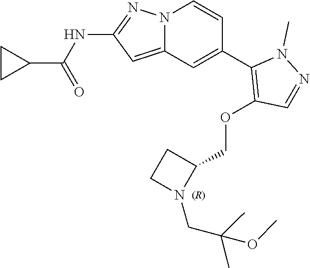

The title compound (9.9 mg, 4.9%) was prepared as described in Example 429, except 2-methoxy-2-methylpropanal was used instead of benzaldehyde. MS (ESI): mass calcd. for C₂₄H₃₂N₆O₃, 452.3. m/z found, 453.1 [M+H]⁺. ¹H NMR (400 MHz, CDCl₃) δ 8.36-8.24 (m, 2H), 7.50-7.46 (m, 1H), 7.33 (s, 1H), 7.00 (s, 1H), 6.87 (dd, J=1.6, 7.2 Hz, 1H), 4.04-3.92 (m, 2H), 3.90 (s, 3H), 3.52-3.41 (m, 2H), 3.09 (s, 3H), 2.94-2.85 (m, 1H), 2.61-2.55 (m, 1H), 2.38-2.32 (m, 1H), 2.05-1.96 (m, 2H), 1.58 (m, 1H), 1.18-1.11 (m, 2H), 1.03 (s, 3H), 1.02 (s, 3H), 0.95-0.88 (m, 2H)

### Example 436: N-[5-[2-methyl-4-[[(2R)-1-(2-pyridyl)azetidin-2-yl]methoxy]pyrazol-3-yl]pyrazolo[1,5-a]pyridin-2-yl]cyclopropanecarboxamide

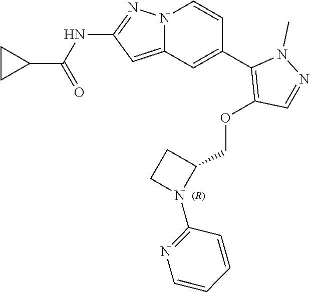

The title compound (59.9 mg, 36%) was prepared as described in Example 429, except 2-fluoropyridine was used instead of benzaldehyde. MS (ESI): mass calcd. for C₂₄H₂₅N₇O₂, 443.2. m/z found, 444.1 [M+H]⁺. ¹H NMR (400 MHz, CDCl₃) δ 8.39 (s, 1H), 8.25 (d, J=7.2 Hz, 1H), 8.13-8.08 (m, 1H), 7.48 (d, J=0.8 Hz, 1H), 7.39 (s, 1H), 7.34-7.28 (m, 1H), 6.99 (s, 1H), 6.83 (dd, J=2.0, 7.2 Hz, 1H), 6.61-6.54 (m, 1H), 6.42-6.35 (m, 1H), 4.59-4.51 (m, 1H), 4.38-4.31 (m, 1H), 4.28-4.21 (m, 1H), 4.02-3.95 (m, 1H), 3.89 (s, 3H), 3.88-3.81 (m, 1H), 2.47-2.31 (m, 2H), 1.63-1.53 (m, 1H), 1.20-1.1 (m, 2H), 0.98-0.86 (m, 2H)

### Example 437: N-[5-[4-[[(2R,3S)-1,3-dimethylazetidin-2-yl]methoxy]-2-methyl-pyrazol-3-yl]pyrazolo[1,5-a]pyridin-2-yl]cyclopropanecarboxamide

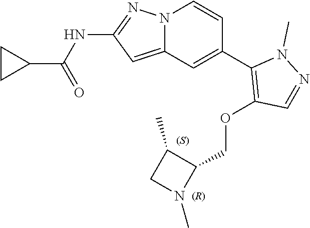

Step A. N-(5-(1-methyl-4-(((2R,3S)-3-methylazetidin-2-yl)methoxy)-1H-pyrazol-5-yl)pyrazolo[1,5-a]pyridin-2-yl)cyclopropanecarboxamide. To a solution of tert-butyl (2R,3S)-2-(hydroxymethyl)-3-methylazetidine-1-carboxylate (80 mg, 0.397 mmol) and DCM (1.655 mL, 1.326 g/mL, 25.836 mmol) was added TsC1 [98-59-9] (0.114 g, 0.596 mmol). The reaction was stirred for 120 min. The crude reaction was diluted with DCM and washed with H2O 2×, 0.2 M aq. HCl 2×, and saturated aqueous NaHCO₃ 2×. The organic layer was collected, dried over anhydrous MgSO4, filtered, and concentrated down under vacuo. The crude was used in the next step without purification. To a separate 0° C. solution of N-(5-(4-hydroxy-1-methyl-1H-pyrazol-5-yl)pyrazolo[1,5-a]pyridin-2-yl)cyclopropanecarboxamide (236.357 mg, 0.795 mmol) and DMF (2.154 mL, 0.944 g/mL, 27.824 mmol) was added NaH (60% dispersion in mineral oil) (39.745 mg, 0.994 mmol). The mixture was stirred for 10 min. before addition of a solution of crude from part 1 and DMF (2.154 mL, 0.944 g/mL, 27.824 mmol). The ice-bath was removed and the mixture was heated to 80 C. The reaction was complete after 1 h. The crude was purified via ISCO using 0-5% MeOH:EtOAc to afford a crude oil. To the crude was added 2 mL of 1:4 TFA:DCM. The reaction was stirred for 1 h. Then, concentrated down under vacuo to a white solid. The crude material was used in the next step.

Step B. N-[5-[4-[[(2R,3S)-1,3-dimethylazetidin-2-yl]methoxy]-2-methyl-pyrazol-3-yl]pyrazolo[1,5-a]pyridin-2-yl]cyclopropanecarboxamide. To a solution of N-(5-(1-methyl-4-(((2R,3S)-3-methylazetidin-2-yl)methoxy)-1H-pyrazol-5-yl)pyrazolo[1,5-a]pyridin-2-yl)cyclopropanecarboxamide (182 mg, 0.368 mmol) and MeOH (1.491 mL, 0.791 g/mL, 36.807 mmol) was added H(CHO)n (0.0265 g, 0.294 mmol) and NaCNBH₃ (0.0694 g, 1.104 mmol) and stirred overnight. The crude material was dissolved with MeOH and purified via ISCO prep. HPLC using 10-60% ACN-H₂O with 20 MM of NH₄OH as modifier. The fractions contained desired material was collected, froze, and lyophilized to afford a white powder (27.9 mg, 18%).

MS (ESI): mass calcd. for C₂₁H₂₆N₆O₂, 394.2. m/z found, 395.2 [M+H]⁺. ¹H NMR (400 MHz, Methanol-d4) δ 8.35 (dt, J=7.2, 0.9 Hz, 1H), 7.53 (dd, J=1.9, 1.0 Hz, 1H), 7.36 (s, 1H), 6.84 (dd, J=7.2, 1.9 Hz, 1H), 6.80 (s, 1H), 4.07-3.92 (m, 2H), 3.79 (s, 3H), 3.44-3.32 (m, 1H), 3.04-2.84 (m, 2H), 2.41 (qd, J=7.5, 2.7 Hz, 1H), 2.22 (s, 3H), 1.78 (tt, J=8.0, 4.7 Hz, 1H), 1.08 (d, J=7.3 Hz, 3H), 0.90 (dt, J=4.6, 3.1 Hz, 2H), 0.85-0.75 (m, 2H).

### Example 438: 3-[[5-[2-[(2,6-dimethylpyrimidin-4-yl)amino]pyrazolo[1,5-a]pyridin-5-yl]-1-methyl-pyrazol-4-yl]oxymethyl]cyclobutanol

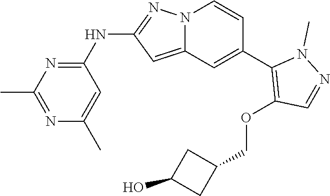 [SMILES: Cc1cc(Nc2cc3cc(-c4c(OC[C@H]5C[C@H](O)C5)cnn4C)ccn3n2)nc(C)n1]

Step A. ((1r,3r)-3-((tert-butyldimethylsilyl)oxy)cyclobutyl)methyl 4-methylbenzenesulfonate. The title compound (1.6 g, 103.8%, impure) was prepared as described in Step A Example 1, except ((trans)-3-((tert-butyldimethylsilyl)oxy)cyclobutyl)methanol was used instead of ( ).

Step B. 3-[[5-[2-[(2,6-dimethylpyrimidin-4-yl)amino]pyrazolo[1,5-a]pyridin-5-yl]-1-methyl-pyrazol-4-yl]oxymethyl]cyclobutanol. The title compound (81.1 mg, 27%) was prepared as described in Example 32 Step A, except Intermediate 10 was used instead of 1-(difluoromethyl)-1H-pyrazol-4-ol and ((1r,3r)-3-((tert-butyldimethylsilyl)oxy)cyclobutyl)methyl 4-methylbenzenesulfonate was used instead of tert-butyl (3S,4S)-3-fluoro-4-(tosyloxy)pyrrolidine-1-carboxylate. MS (ESI): mass calcd. for C₂₂H₂₅N₇O₂, 419.2. m/z found, 420.1 [M+H]⁺. ¹H NMR (400 MHz, CDCl₃) δ 8.40 (d, J=7.6 Hz, 1H), 7.57 (s, 1H), 7.46 (d, J=1.2 Hz, 1H), 7.34 (s, 1H), 7.06 (s, 1H), 6.84 (dd, J=2.0, 7.2 Hz, 1H), 6.60 (s, 1H), 4.49-4.35 (m, 1H), 3.95-3.94 (m, 1H), 3.93-3.92 (m, 1H), 3.92 (s, 3H), 2.67-2.55 (m, 4H), 2.45 (s, 3H), 2.28-2.19 (m, 2H), 2.15-2.01 (m, 3H)

### Example 439: (*S)-3-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol

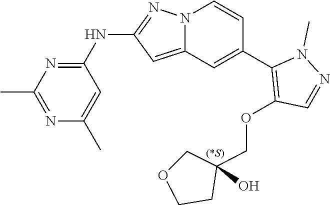

Step A. (3-hydroxytetrahydrofuran-3-yl)methyl 4-methylbenzenesulfonate. The title compound (310 mg, 67%) was prepared as described in Example 9 Step A, except 3-(hydroxymethyl)tetrahydrofuran-3-ol was used instead of tert-butyl (S)-2-(hydroxymethyl)azetidine-1-carboxylate.

Step B. 3-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol. The title compound was prepared as described in Example 9Step B, except Intermediate 10 was used instead of Intermediate 4 and (3-hydroxytetrahydrofuran-3-yl)methyl 4-methylbenzenesulfonate was used instead of tert-butyl (S)-2-((tosyloxy)methyl)azetidine-1-carboxylate. The material was further purified by SFC over DAICEL CHIRALCEL OD-H(250 mm×30 mm, 5 um), (eluent: 45% to 45% (v/v) supercritical CO₂ in EtOH and H₂O with 0.1% NH₃). The product was second to elute at 2.30 min. The pure fractions were collected and the volatiles were removed under reduced pressure. The product was suspended in water (10 mL), the mixture frozen using dry ice/ethanol, and then lyophilized to dryness to afford the product (45.4 mg, 12%), second to elute, as a white solid. MS (ESI): mass calcd. for C₂₂H₂₅N₇O₃, 435.2. m/z found, 436.1 [M+H]⁺. ¹H NMR (400 MHz, DMSO-d6) δ 10.23 (s, 1H), 8.62 (d, J=7.2 Hz, 1H), 7.73 (s, 1H), 7.47 (s, 1H), 7.01 (br. s., 1H), 6.97 (dd, J=1.6, 7.2 Hz, 1H), 6.88 (br. s., 1H), 5.13 (s, 1H), 3.99-3.92 (m, 2H), 3.88 (s, 3H), 3.86-3.80 (m, 1H), 3.78-3.71 (m, 1H), 3.67 (d, J=8.8 Hz, 1H), 3.55 (d, J=8.8 Hz, 1H), 2.46 (s, 3H), 2.30 (s, 3H), 2.02-1.91 (m, 1H), 1.85-1.76 (m, 1H)

### Example 440: (*S)—N-(2,6-dimethylpyrimidin-4-yl)-5-[2-methyl-4-[(3-methyltetrahydrofuran-3-yl)methoxy]pyrazol-3-yl]pyrazolo[1,5-a]pyridin-2-amine

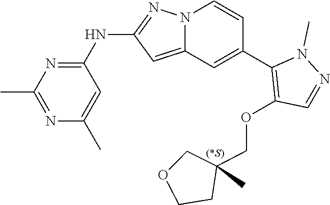

Step A. (3-methyltetrahydrofuran-3-yl)methyl 4-methylbenzenesulfonate. The title compound (310 mg, 67%) was prepared as described in Example 9 Step A, except (3-methyltetrahydrofuran-3-yl)methanol was used instead of tert-butyl (S)-2-(hydroxymethyl)azetidine-1-carboxylate.

Step B. N-(2,6-dimethylpyrimidin-4-yl)-5-[2-methyl-4-[(3-methyltetrahydrofuran-3-yl)methoxy]pyrazol-3-yl]pyrazolo[1,5-a]pyridin-2-amine. The title compound (280 mg, 61%) was prepared as described in Example 9 Step B, except Intermediate 10 was used instead of Intermediate 4 and (3-methyltetrahydrofuran-3-yl)methyl 4-methylbenzenesulfonate was used instead of tert-butyl (S)-2-((tosyloxy)methyl)azetidine-1-carboxylate. The material (280 mg) was further purified by SFC over DAICEL CHIRALCEL OD-H(250 mm×30 mm, 5 um), (eluent: 45% to 45% (v/v) supercritical CO₂ in EtOH and H₂O with 0.1% NH₃). The pure fractions were collected and the volatiles were removed under reduced pressure. The product was suspended in water (10 mL), the mixture frozen using dry ice/ethanol, and then lyophilized to dryness to afford the product (45.8 mg, 16.4%), second to elute, as a white solid. MS (ESI): mass calcd. for C₂₃H₂₇N₇O₂, 433.2. m/z found, 434.1 [M+H]⁺. ¹H NMR (400 MHz, CDCl₃) δ 8.42 (d, J=7.2 Hz, 1H), 7.58 (s, 1H), 7.45 (d, J=1.2 Hz, 1H), 7.35 (s, 1H), 7.07 (s, 1H), 6.85 (dd, J=2.0, 7.2 Hz, 1H), 6.60 (s, 1H), 3.93 (s, 3H), 3.89-3.84 (m, 2H), 3.84-3.77 (m, 2H), 3.76 (d, J=8.4 Hz, 1H), 3.42 (d, J=8.8 Hz, 1H), 2.61 (s, 3H), 2.45 (s, 3H), 1.93-1.84 (m, 1H), 1.70-1.62 (m, 1H), 1.20 (s, 3H)

### Example 441: (1*R, 2*S, 4*S)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine

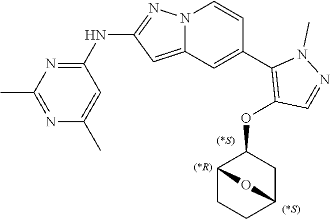

Step A. (1R,2R,4S)-7-oxabicyclo[2.2.1]heptan-2-yl 4-methylbenzenesulfonate. The title compound (1.9 g, 89.8%) was prepared as described in Example 9 Step A, except (1R,2R,4S)-7-oxabicyclo[2.2.1]heptan-2-ol was used instead of tert-butyl (S)-2-(hydroxymethyl)azetidine-1-carboxylate.

Step B. (1*R, 2*S, 4*S)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine. The title compound (60 mg, 23%) was prepared as described in Example 1 Step B, except Intermediate 10 was used instead of Intermediate 4 and (1R,2R,4S)-7-oxabicyclo[2.2.1]heptan-2-yl 4-methylbenzenesulfonate was used instead of tert-butyl (S)-2-((tosyloxy)methyl)azetidine-1-carboxylate. The material was purified by SFC-21 over DAICEL CHIRALPAK IG 250 mm×30 mm×10 μm (eluent: 55% to 55% (v/v) EtOH with 0.1% NH₃·H₂O in supercritical CO₂). The product was second to elute at 2.23 min. The pure fractions were collected and the volatiles were removed under reduced pressure to afford the product (23.5 mg, 8% overall) as an off-white solid. MS (ESI): mass calcd. for C₂₃H₂₅N₇O₂, 431.2. m/z found, 432.1 [M+H]⁺. ¹H NMR (400 MHz, DMSO-d6) δ 10.23 (s, 1H), 8.64 (d, J=7.2 Hz, 1H), 7.73-7.64 (m, 1H), 7.45 (s, 1H), 7.01 (br. s., 1H), 6.94-6.85 (m, 2H), 4.68-4.62 (m, 1H), 4.54-4.45 (m, 2H), 3.86 (s, 3H), 2.47 (s, 3H), 2.30 (s, 3H), 2.18-2.10 (m, 1H), 2.06-1.99 (m, 1H), 1.67-1.57 (m, 1H), 1.55-1.43 (m, 2H), 1.27 (dd, J=2.8, 12.8 Hz, 1H)

### Example 442: N-(2,6-dimethylpyrimidin-4-yl)-5-[2-methyl-4-[(2R,3S)-2-methylpyrrolidin-3-yl]oxy-pyrazol-3-yl]pyrazolo[1,5-a]pyridin-2-amine

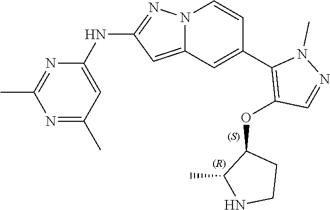

Step A. Tert-butyl (2R,3S)-3-((5-(2-((2,6-dimethylpyrimidin-4-yl)amino)pyrazolo[1,5-a]pyridin-5-yl)-1-methyl-1H-pyrazol-4-yl)oxy)-2-methylpyrrolidine-1-carboxylate. The title compound (249.9 mg, 26% overall) was prepared as described in Example 441 Step A-B, except cis-tert-butyl 3-hydroxy-2-methylpyrrolidine-1-carboxylate was used instead of (1R,2R,4S)-7-oxabicyclo[2.2.1]heptan-2-ol in Step A. The pure was further purified by SFC over DAICEL CHIRALCEL OD-H(250 mm×30 mm, 5 um), (eluent: 35% to 35% (v/v) supercritical CO₂ in EtOH and H₂O with 0.1% NH3H2O IPA). The product was second to elute at 5.76 min. The pure fractions were collected and the volatiles were removed under reduced pressure. The product was suspended in water (10 mL), the mixture frozen using dry ice/ethanol, and then lyophilized to dryness to afford the product as a white solid.

Step B. N-(2,6-dimethylpyrimidin-4-yl)-5-[2-methyl-4-[(2R,3S)-2-methylpyrrolidin-3-yl]oxy-pyrazol-3-yl]pyrazolo[1,5-a]pyridin-2-amine. The title compound (42 mg, 27%) was prepared as described in Step B of Example 2, except tert-butyl (2R,3S)-3-((5-(2-((2,6-dimethylpyrimidin-4-yl)amino)pyrazolo[1,5-a]pyridin-5-yl)-1-methyl-1H-pyrazol-4-yl)oxy)-2-methylpyrrolidine-1-carboxylate was used instead of tert-butyl (R)-2-(((5-(2-((2,6-dimethylpyrimidin-4-yl)amino)pyrazolo[1,5-a]pyridin-5-yl)-1-methyl-1H-pyrazol-4-yl)oxy)methyl)azetidine-1-carboxylate. MS (ESI): mass calcd. for C₂₂H₂₆N₈O, 418.2. m/z found, 419.1 [M+H]⁺. ¹H NMR (400 MHz, CDCl₃) δ 8.41 (d, J=7.2 Hz, 1H), 7.72 (s, 1H), 7.45 (s, 1H), 7.30 (s, 1H), 7.04 (s, 1H), 6.83 (dd, J=1.2, 6.8 Hz, 1H), 6.62 (s, 1H), 4.21-4.16 (m, 1H), 3.92 (s, 3H), 3.35-3.27 (m, 1H), 3.06-2.99 (m, 2H), 2.61 (s, 3H), 2.45 (s, 3H), 2.09-1.99 (m, 1H), 1.97-1.93 (m, 1H), 1.13 (d, J=6.8 Hz, 3H)

### Example 443: (*S)—N-(2,6-dimethylpyrimidin-4-yl)-5-[2-methyl-4-(8-oxaspiro[4.4]nonan-7-ylmethoxy)pyrazol-3-yl]pyrazolo[1,5-a]pyridin-2-amine

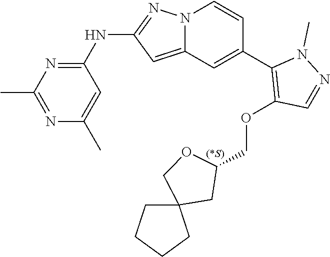

Step A. (2-oxaspiro[4.4]nonan-3-yl)methyl 4-methylbenzenesulfonate. The title compound (310 mg, 78%) was prepared as described in Example 9 Step A, except 2-oxaspiro[4.4]nonan-3-ylmethanol was used instead of tert-butyl (S)-2-(hydroxymethyl)azetidine-1-carboxylate.

Step B. N-(2,6-dimethylpyrimidin-4-yl)-5-[2-methyl-4-(8-oxaspiro[4.4]nonan-7-ylmethoxy)pyrazol-3-yl]pyrazolo[1,5-a]pyridin-2-amine. The title compound was prepared as described in Example 1 Step B, except Intermediate 10 was used instead of Intermediate 4 and (2-oxaspiro[4.4]nonan-3-yl)methyl 4-methylbenzenesulfonate was used instead of tert-butyl (S)-2-((tosyloxy)methyl)azetidine-1-carboxylate. The material was further purified by SFC over DAICEL CHIRALPAK AD(250 mm×30 mm, 10 um), (eluent: 55% to 55% (v/v) supercritical CO₂ in EtOH and H₂O with 0.1% NH₃H₂O). The product was second to elute at 2.68 min. The pure fractions were collected and the volatiles were removed under reduced pressure. The product was suspended in water (10 mL), the mixture frozen using dry ice/ethanol, and then lyophilized to dryness to afford the title compound (87.9 mg, 13%, 2 steps) as a white solid. MS (ESI): mass calcd. for C₂₆H₃₁N₇O₂, 473.3. m/z found, 474.2 [M+H]⁺. ¹H NMR (400 MHz, DMSO-d6) δ 10.22 (s, 1H), 8.62 (d, J=7.2 Hz, 1H), 7.69 (d, J=0.8 Hz, 1H), 7.46 (s, 1H), 6.99 (s, 1H), 6.94 (dd, J=2.0, 7.2 Hz, 1H), 6.89 (s, 1H), 4.24-4.13 (m, 1H), 4.02-3.91 (m, 2H), 3.87 (s, 3H), 3.52-3.49 (m, 1H), 3.47-3.42 (m, 1H), 2.46 (s, 3H), 2.30 (s, 3H), 1.91-1.82 (m, 1H), 1.66-1.46 (m, 9H)

### Example 444: (*R)-1-cyclobutyl-2-[5-[2-[(2,6-dimethylpyrimidin-4-yl)amino]pyrazolo[1,5-a]pyridin-5-yl]-1-methyl-pyrazol-4-yl]oxy-ethanol

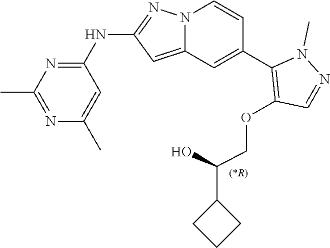

TEA (241 mg, 2.39 mmol) was added to a solution of Intermediate 10 (400 mg, 1.19 mmol), 2-cyclobutyloxirane (234 mg, 2.39 mmol) and MeOH (15 mL). The reaction was stirred at 80° C. for 12 hours. The mixture was purified by preparative HPLC using a Phenomenex Gemini NX-C18 75×30 mm×3 um column (eluent: 27% to 57% (v/v) CH3CN and H2O with 0.05% NH₃) to afford pure product. The product was further purified by SFC over Phenomenex-Cellulose-2 250 mm×30 mm, 10 μm (eluent: 50% to 50% (v/v) supercritical CO2 in EtOH and H2O with 0.1% NH3). The title compound was first to elute at 3.32 min. The pure fractions were collected and the volatiles were removed under reduced pressure. The product was suspended in water (10 mL), the mixture frozen using dry ice/acetone, and then lyophilized to dryness to afford the title compound as a white solid (54.1 mg, 10.4%, 1 step). MS (ESI): mass calcd. for C₂₃H₂₇N₇O₂, 433.2. m/z found, 434.1 [M+H]⁺. ¹H NMR (400 MHz, DMSO-d6) δ 10.22 (s, 1H), 8.61 (d, J=7.2 Hz, 1H), 7.71 (d, J=1.2 Hz, 1H), 7.42 (s, 1H), 7.02-6.95 (m, 2H), 6.89 (br. s., 1H), 4.90-4.79 (m, 1H), 3.87 (s, 3H), 3.80-3.71 (m, 2H), 3.68-3.59 (m, 1H), 2.46 (s, 3H), 2.42-2.35 (m, 1H), 2.31 (s, 3H), 1.93-1.76 (m, 5H), 1.75-1.65 (m, 1H)

### Example 445: (3*S,4*S)—N-(2,6-dimethylpyrimidin-4-yl)-5-[2-methyl-4-(4-methyltetrahydrofuran-3-yl)oxy-pyrazol-3-yl]pyrazolo[1,5-a]pyridin-2-amine

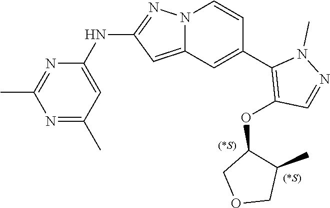

Step A. (3S,4R)-4-methyltetrahydrofuran-3-yl 4-methylbenzenesulfonate. The title compound (310 mg, 78%) was prepared as described in Example 9 Step A, except (3S,4R)-4-methyltetrahydrofuran-3-ol was used instead of tert-butyl (S)-2-(hydroxymethyl)azetidine-1-carboxylate.

Step B. (3*S,4*S)—N-(2,6-dimethylpyrimidin-4-yl)-5-[2-methyl-4-(4-methyltetrahydrofuran-3-yl)oxy-pyrazol-3-yl]pyrazolo[1,5-a]pyridin-2-amine. The title compound was prepared as described in Example 1 Step B, except Intermediate 10 was used instead of Intermediate 4 and (3S,4R)-4-methyltetrahydrofuran-3-yl 4-methylbenzenesulfonate was used instead of tert-butyl (S)-2-((tosyloxy)methyl)azetidine-1-carboxylate. The material was further purified by SFC over DAICEL CHIRALPAK IG 250 mm×30 mm, 10 μm (eluent: 55% to 55% (v/v) supercritical CO₂ in EtOH and H₂O with 0.1% NH₃). The title compound was second to elute at 1.600 min. The pure fractions were collected and the volatiles were removed under reduced pressure. The product was suspended in water (10 mL), the mixture frozen using dry ice/acetone, and then lyophilized to dryness to afford the title compound (82 mg, 11%, 2 steps) as a white solid. MS (ESI): mass calcd. for C₂₂H₂₅N₇O₂, 419.2. m/z found, 420.1 [M+H]⁺. ¹H NMR (400 MHz, DMSO-d6) δ 10.22 (s, 1H), 8.63 (d, J=7.2 Hz, 1H), 7.69 (d, J=0.8 Hz, 1H), 7.43 (s, 1H), 7.00 (br. s., 1H), 6.92 (dd, J=2.0, 7.2 Hz, 1H), 6.88 (br. s., 1H), 4.62-4.57 (m, 1H), 3.93-3.86 (m, 5H), 3.80-3.75 (m, 1H), 3.38-3.35 (m, 1H), 2.46 (s, 3H), 2.41-2.35 (m, 1H), 2.30 (s, 3H), 1.02 (d, J=6.8 Hz, 3H)

### Example 446: N-(2,6-dimethylpyrimidin-4-yl)-5-[2-methyl-4-[[(2R,4S)-4-methylpyrrolidin-2-yl]methoxy]pyrazol-3-yl]pyrazolo[1,5-a]pyridin-2-amine

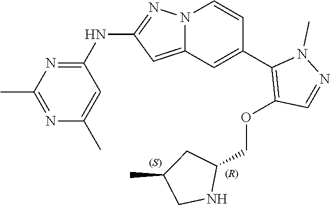

Step A. Tert-butyl (2R,4S)-4-methyl-2-((tosyloxy)methyl)pyrrolidine-1-carboxylate. The title compound (360 mg, 105%, impure) was prepared as described in Example 9 Step A, except (2R,4S)-tert-butyl 2-(hydroxymethyl)-4-methylpyrrolidine-1-carboxylate was used instead of tert-butyl (S)-2-(hydroxymethyl)azetidine-1-carboxylate.

Step B. N-(2,6-dimethylpyrimidin-4-yl)-5-[2-methyl-4-[[(2R,4S)-4-methylpyrrolidin-2-yl]methoxy]pyrazol-3-yl]pyrazolo[1,5-a]pyridin-2-amine. The title compound (12.9 mg, 2 steps, 2.7%) was prepared as described in Example 1 Step B and Step E, except Intermediate 10 was used instead of 1-methyl-1h-pyrazol-4-ol in Step B, tert-butyl (2R,4S)-4-methyl-2-((tosyloxy)methyl)pyrrolidine-1-carboxylate was used instead of (2S,4R)-2-methyl-4-(tosyloxy)pyrrolidine-1-carboxylate in Step B, (2R,4S)-tert-butyl 2-(((5-(2-((2,6-dimethylpyrimidin-4-yl)amino)pyrazolo[1,5-a]pyridin-5-yl)-1-methyl-1H-pyrazol-4-yl)oxy)methyl)-4-methylpyrrolidine-1-carboxylate was used instead of tert-butyl (2S,4S)-4-((5-(2-(cyclopropanecarboxamido)pyrazolo[1,5-a]pyridin-5-yl)-1-methyl-1H-pyrazol-4-yl)oxy)-2-methylpyrrolidine-1-carboxylate in Step E, and dichloromethane (6 mL) was used instead of dichloroethane in Step E. MS (ESI): mass calcd. for C₂₃H₂₈N₈O, 432.2. m/z found, 433.1 [M+H]⁺. ¹H NMR (400 MHz, DMSO-d6) δ 10.21 (s, 1H), 8.61 (d, J=7.2 Hz, 1H), 7.70 (d, J=1.2 Hz, 1H), 7.43 (s, 1H), 6.99 (s, 1H), 6.95 (dd, J=2.0, 7.2 Hz, 1H), 6.89 (s, 1H), 3.86 (s, 3H), 3.80-3.71 (m, 2H), 3.43-3.41 (m, 1H), 2.97-2.91 (m, 1H), 2.46 (s, 3H), 2.36-2.32 (m, 1H), 2.30 (s, 3H), 2.14-2.02 (m, 1H), 1.68-1.60 (m, 1H), 1.40-1.32 (m, 1H), 0.93 (d, J=6.8 Hz, 3H)

### Example 447: (*S)—N-(2,6-dimethylpyrimidin-4-yl)-5-[2-methyl-4-[(2-methyltetrahydrofuran-2-yl)methoxy]pyrazol-3-yl]pyrazolo[1,5-a]pyridin-2-amine

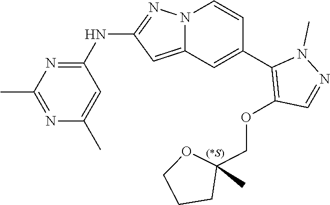

Step A. (2-methyltetrahydrofuran-2-yl)methyl 4-methylbenzenesulfonate. The title compound (1.7 g, 81%) was prepared as described in Example 9 Step A, except (2-methyltetrahydrofuran-2-yl)methanol was used instead of tert-butyl (S)-2-(hydroxymethyl)azetidine-1-carboxylate.

Step B. (*S)—N-(2,6-dimethylpyrimidin-4-yl)-5-[2-methyl-4-[(2-methyltetrahydrofuran-2-yl)methoxy]pyrazol-3-yl]pyrazolo[1,5-a]pyridin-2-amine. The title compound was prepared as described in Example 1 Step B, except Intermediate 10 was used instead of Intermediate 4 and (2-methyltetrahydrofuran-2-yl)methyl 4-methylbenzenesulfonate was used instead of tert-butyl (S)-2-((tosyloxy)methyl)azetidine-1-carboxylate. The material was further purified by SFC over DAICEL CHIRALPAK IG 250 mm×30 mm, 10 μm (eluent: 55% to 55% (v/v) supercritical C₀₂ in EtOH and H₂O with 0.1% NH₃). The title compound eluted second at 2.33 min. The pure fractions were collected and the volatiles were removed under reduced pressure. The product was suspended in water (10 mL), the mixture frozen using dry ice/acetone, and then lyophilized to dryness to afford the title compound (26.3 mg, 4%, 2 steps) as a white solid. MS (ESI): mass calcd. for C₂₃H₂₇N₇O₂, 433.2. m/z found, 434.1 [M+H]⁺. ¹H NMR (400 MHz, DMSO-d6) δ 10.23 (s, 1H), 8.63 (d, J=6.8 Hz, 1H), 7.70 (d, J=1.2 Hz, 1H), 7.45 (s, 1H), 7.01 (s, 1H), 6.95 (dd, J=2.0, 7.2 Hz, 1H), 6.87 (s, 1H), 3.88 (s, 3H), 3.84-3.77 (m, 2H), 3.77-3.66 (m, 2H), 2.46 (s, 3H), 2.30 (s, 3H), 1.96-1.80 (m, 3H), 1.65-1.56 (m, 1H), 1.19 (s, 3H)

### Example 448: (1*S,2*S)-2-[[5-[2-[(2,6-dimethylpyrimidin-4-yl)amino]pyrazolo[1,5-a]pyridin-5-yl]-1-methyl-pyrazol-4-yl]oxymethyl]cyclobutanol

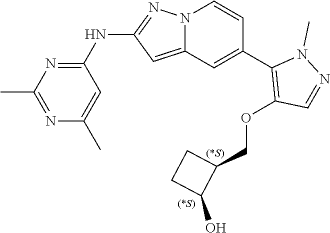

The title compound (26 mg, 6%) was prepared as described in Example 389 Step A, except Intermediate 10 was used instead of tert-butyl (2S,4RS)-2-(difluoromethyl)-4-hydroxypyrrolidine-1-carboxylate and (1r,2r)-2-(hydroxymethyl)cyclobutan-1-ol was used instead of 4-bromo-5-methylisoxazol-3-ol. The material was further purified via SFC purification and the preparative method was AD-H (3×25 cm), 35% ethanol (0.1% DEA)/CO2, 100 bar, 70 mL/min, 220 nm, inj vol.: 1 mL, 4 mg/mL ethanol:DCM. The title compound was second to elute. MS (ESI): mass calcd. for C₂₂H₂₅N₇O₂, 419.2. m/z found, 420.2 [M+H]⁺. ¹H NMR (500 MHz, DMSO-d6) δ 10.19 (s, 1H), 8.59 (dt, J=7.2, 1.0 Hz, 1H), 7.69 (dd, J=2.0, 0.9 Hz, 1H), 7.43 (s, 1H), 7.00 (s, 1H), 6.94 (dd, J=7.2, 2.0 Hz, 1H), 6.86 (s, 1H), 4.94 (d, J=5.5 Hz, 1H), 4.26 (p, J=7.1 Hz, 1H), 4.18 (dd, J=9.9, 6.2 Hz, 1H), 4.06 (dd, J=9.9, 8.6 Hz, 1H), 3.86 (s, 3H), 2.74-2.63 (m, 1H), 2.46 (s, 3H), 2.30 (s, 3H), 2.19-2.08 (m, 1H), 2.01-1.88 (m, 1H), 1.67-1.50 (m, 2H).

### Example 449: 5-[2-methyl-4-[(3S)-1-methylpyrrolidin-3-yl]oxy-pyrazol-3-yl]-N-pyridazin-3-yl-pyrazolo[1,5-a]pyridin-2-amine

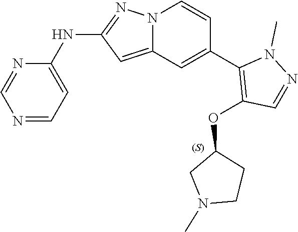

Step A. Tert-butyl (R)-3-(tosyloxy)pyrrolidine-1-carboxylate. The title compound (32 g, 79%) was prepared as described in Example 1 Step A, except (R)-1-N-Boc-3-hydroxypyrrolidine was used instead of tert-butyl (S)-2-(hydroxymethyl)azetidine-1-carboxylate.

Step B. Tert-butyl (S)-3-((1-methyl-1H-pyrazol-4-yl)oxy)pyrrolidine-1-carboxylate. The title compound (14.36 g, 64%, 2 steps) was prepared as described in Example 1 Steps B-C, except tert-butyl (R)-3-(tosyloxy)pyrrolidine-1-carboxylate was used instead of (2S,4R)-2-methyl-4-(tosyloxy)pyrrolidine-1-carboxylate in Step B and (S)-tert-butyl 3-((1-methyl-1H-pyrazol-4-yl)oxy)pyrrolidine-1-carboxylate was used instead of tert-butyl (2S,4S)-2-methyl-4-((1-methyl-1H-pyrazol-4-yl)oxy)pyrrolidine-1-carboxylate in Step C.

Step C. (S)-tert-butyl 3-((5-(2-((tert-butoxycarbonyl)amino)pyrazolo[1,5-a]pyridin-5-yl)-1-methyl-1H-pyrazol-4-yl)oxy)pyrrolidine-1-carboxylate. The title compound (26 g, 103%, impure) was prepared as described in Example 5 Step D, except tert-butyl (5-(4,4,5,5-tetramethyl-1,3,2-dioxaborolan-2-yl)pyrazolo[1,5-a]pyridin-2-yl)carbamate was used instead of Intermediate 3 and tert-butyl (S)-3-((I-methyl-1H-pyrazol-4-yl)oxy)pyrrolidine-1-carboxylate was used instead of rac-cis-tert-butyl (3s,4r)-3-((5-bromo-1-methyl-1H-pyrazol-4-yl)oxy)-4-methoxypyrrolidine-1-carboxylate.

Step D. (S)-5-(1-methyl-4-(pyrrolidin-3-yloxy)-1H-pyrazol-5-yl)pyrazolo[1,5-a]pyridin-2-amine. The title compound (9.2 g, 77%) was prepared as described in Example 6 Step C, except (S)-tert-butyl 3-((5-(2-((tert-butoxycarbonyl)amino)pyrazolo[1,5-a]pyridin-5-yl)-1-methyl-1H-pyrazol-4-yl)oxy)pyrrolidine-1-carboxylate was used instead of (R)-tert-butyl 2-(((5-(2-(cyclopropanecarboxamido)pyrazolo[1,5-a]pyridin-5-yl)-1-methyl-1H-pyrazol-4-yl)oxy)methyl)morpholine-4-carboxylate.

Step E. (S)-5-(1-methyl-4-((1-methylpyrrolidin-3-yl)oxy)-1H-pyrazol-5-yl)pyrazolo[1,5-a]pyridin-2-amine. The title compound (4.1 g, 39%) was prepared as described in Example 8 Step B, except (S)-5-(1-methyl-4-(pyrrolidin-3-yloxy)-1H-pyrazol-5-yl)pyrazolo[1,5-a]pyridin-2-amine was used instead of N-(5-(4-(azetidin-3-ylmethoxy)-1-methyl-1H-pyrazol-5-yl)pyrazolo[1,5-a]pyridin-2-yl)cyclopropanecarboxamide.

Step F. 5-[2-methyl-4-[(3S)-1-methylpyrrolidin-3-yl]oxy-pyrazol-3-yl]-N-pyridazin-3-yl-pyrazolo[1,5-a]pyridin-2-amine. The title compound (66 mg, 35%) was prepared as described in Example 7, except 3-bromopyridazine was used instead of 2-chloro-6-(trifluoromethyl)pyrazine. MS (ESI): mass calcd. for C₂₀H₂₂N₈O, 390.2. m/z found, 391.2 [M+H]⁺. ¹H NMR (400 MHz, DMSO-d6) δ 10.20 (s, 1H), 8.73 (dd, J=1.2, 4.4 Hz, 1H), 8.61 (d, J=7.6 Hz, 1H), 7.69 (d, J=0.8 Hz, 1H), 7.64 (dd, J=1.6, 9.2 Hz, 1H), 7.52 (dd, J=4.4, 8.8 Hz, 1H), 7.40 (s, 1H), 6.95 (s, 1H), 6.91 (dd, J=2.0, 7.2 Hz, 1H), 4.71-4.63 (m, 1H), 3.86 (s, 3H), 2.70-2.64 (m, 1H), 2.63-2.57 (m, 2H), 2.34-2.26 (m, 1H), 2.21 (s, 3H), 2.20-2.12 (m, 1H), 1.82-1.71 (m, 1H)

### Example 450: 5-[2-methyl-4-[(3S)-1-methylpyrrolidin-3-yl]oxy-pyrazol-3-yl]-N-pyrazin-2-yl-pyrazolo[1,5-a]pyridin-2-amine

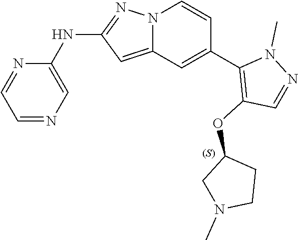

The title compound was prepared as described in Example 7, except 2-bromopyrazine was used instead of 2-chloro-6-(trifluoromethyl)pyrazine. MS (ESI): mass calcd. for C₂₀H₂₂N₈O, 390.2. m/z found, 391.2 [M+H]⁺. ¹H NMR (400 MHz, DMSO-d6) δ 10.30 (s, 1H), 8.65-8.59 (m, 2H), 8.24-8.19 (m, 1H), 8.01 (d, J=2.8 Hz, 1H), 7.68 (d, J=0.8 Hz, 1H), 7.39 (s, 1H), 6.94 (s, 1H), 6.92 (dd, J=2.0, 6.8 Hz, 1H), 4.70-4.63 (m, 1H), 3.86 (s, 3H), 2.71-2.63 (m, 1H), 2.63-2.56 (m, 2H), 2.35-2.25 (m, 1H), 2.24-2.12 (m, 4H), 1.83-1.72 (m, 1H)

### Example 451: 5-[2-methyl-4-[(3S)-1-methylpyrrolidin-3-yl]oxy-pyrazol-3-yl]-N-(6-methyl-3-pyridyl)pyrazolo[1,5-a]pyridin-2-amine

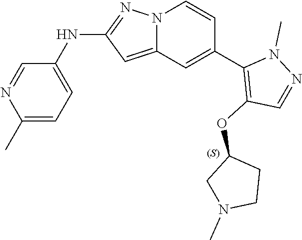

The title compound (46 mg, 23%) was prepared as described in Example 7, except 5-bromo-2-methylpyridine was used instead of 2-chloro-6-(trifluoromethyl)pyrazine. MS (ESI): mass calcd. for C₂₂H₂₅N₇O, 403.2. m/z found, 404.2 [M+H]⁺. ¹H NMR (400 MHz, DMSO-d6) δ 9.09 (s, 1H), 8.62-8.55 (m, 2H), 7.94 (dd, J=2.8, 8.4 Hz, 1H), 7.54 (d, J=0.8 Hz, 1H), 7.39 (s, 1H), 7.15 (d, J=8.4 Hz, 1H), 6.81 (dd, J=1.6, 6.8 Hz, 1H), 6.12 (s, 1H), 4.69-4.62 (m, 1H), 3.84 (s, 3H), 2.69-2.63 (m, 1H), 2.63-2.56 (m, 2H), 2.39 (s, 3H), 2.34-2.25 (m, 1H), 2.21 (s, 3H), 2.20-2.12 (m, 1H), 1.81-1.71 (m, 1H)

### Example 452: 5-[2-methyl-4-[(3S)-1-methylpyrrolidin-3-yl]oxy-pyrazol-3-yl]-N-(2-pyridyl)pyrazolo[1,5-a]pyridin-2-amine

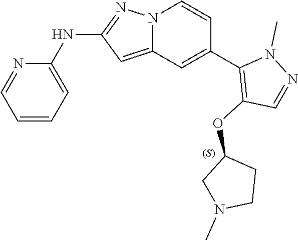

The title compound (46.7 mg, 30%) was prepared as described in Example 7, except 2-bromopyridine was used instead of 2-chloro-6-(trifluoromethyl)pyrazine. MS (ESI): mass calcd. for C₂₁H₂₃N₇O, 389.2. m/z found, 390.2 [M+H]⁺. ¹H NMR (400 MHz, DMSO-d6) δ 9.83 (s, 1H), 8.57 (d, J=7.2 Hz, 1H), 8.24-8.18 (m, 1H), 7.67-7.58 (m, 2H), 7.39 (s, 1H), 7.32 (d, J=8.0 Hz, 1H), 6.90 (s, 1H), 6.86 (dd, J=2.0, 7.2 Hz, 1H), 6.83-6.77 (m, 1H), 4.70-4.63 (m, 1H), 3.85 (s, 3H), 2.70-2.63 (m, 1H), 2.63-2.57 (m, 2H), 2.34-2.26 (m, 1H), 2.21 (s, 3H), 2.20-2.13 (m, 1H), 1.82-1.72 (m, 1H)

### Example 453: 5-[2-methyl-4-[(3S)-1-methylpyrrolidin-3-yl]oxy-pyrazol-3-yl]-N-[5-(trifluoromethyl)pyrazin-2-yl]pyrazolo[1,5-a]pyridin-2-amine

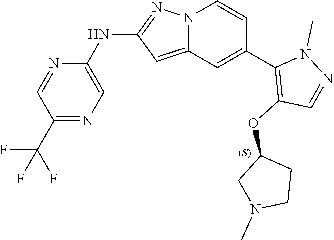

The title compound (62.9 mg, 27%) was prepared as described in Example 7, except 2-chloro-5-(trifluoromethyl)pyrazine was used instead of 2-chloro-6-(trifluoromethyl)pyrazine. MS (ESI): mass calcd. for C₂₁H₂₁F₃N₈O, 458.2. m/z found, 459.27 [M+H]⁺. ¹H NMR (400 MHz, DMSO-d6) δ8.68-8.64 (m, 2H), 8.61 (s, 1H), 7.73 (d, J=1.2 Hz, 1H), 7.40 (s, 1H), 7.05 (s, 1H), 6.97 (dd, J=2.0, 7.2 Hz, 1H), 4.72-4.63 (m, 1H), 3.86 (s, 3H), 2.70-2.64 (m, 1H), 2.64-2.57 (m, 2H), 2.33-2.24 (m, 1H), 2.21 (s, 3H), 2.20-2.13 (m, 1H), 1.83-1.71 (m, 1H) 19F NMR (376 MHz, DMSO-d6) δ −64.85 (s, 3F)

### Example 454: 5-[2-methyl-4-[(3S)-1-methylpyrrolidin-3-yl]oxy-pyrazol-3-yl]-N-(5-methylpyrazin-2-yl)pyrazolo[1,5-a]pyridin-2-amine

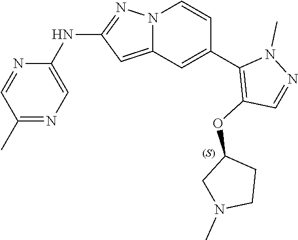

The title compound (69.3 mg, 51%) was prepared as described in Example 7, except 2-bromo-5-methylpyrazine was used instead of 2-chloro-6-(trifluoromethyl)pyrazine. MS (ESI): mass calcd. for C₂₁H₂₄N₈O, 404.2. m/z found, 405.2 [M+H]⁺. ¹H NMR (400 MHz, DMSO-d6) δ 9.49 (s, 1H), 8.49 (d, J=7.2 Hz, 1H), 8.28 (s, 1H), 7.92 (d, J=1.2 Hz, 1H), 7.64 (s, 1H), 7.36 (s, 1H), 7.04 (dd, J=2.0, 7.2 Hz, 1H), 6.25 (s, 1H), 4.74-4.60 (m, 1H), 4.04 (s, 3H), 3.69-3.62 (m, 2H), 3.61-3.56 (m, 2H), 3.10 (d, J=7.2 Hz, 2H), 2.46 (s, 3H), 2.38-2.30 (m, 2H), 1.83-1.73 (m, 2H)

### Example 455: 5-[2-methyl-4-[(3S)-1-methylpyrrolidin-3-yl]oxy-pyrazol-3-yl]-N-pyrimidin-4-yl-pyrazolo[1,5-a]pyridin-2-amine

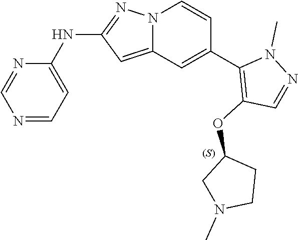

The title compound (17.4 mg, 8.5%) was prepared as described in Example 7, except 4-chloropyrimidine hydrogenchloride was used instead of 2-chloro-6-(trifluoromethyl)pyrazine. MS (ESI): mass calcd. for C₂₀H₂₂N₈O, 390.2. m/z found, 391.2 [M+H]⁺. ¹H NMR (400 MHz, DMSO-d6) δ 10.44 (s, 1H), 8.70 (s, 1H), 8.63 (d, J=7.2 Hz, 1H), 8.38 (d, J=6.0 Hz, 1H), 7.72-7.69 (m, 1H), 7.40 (s, 1H), 7.31 (d, J=4.8 Hz, 1H), 6.98-6.88 (m, 2H), 4.72-4.64 (m, 1H), 3.85 (s, 3H), 2.77-2.63 (m, 3H), 2.42-2.35 (m, 1H), 2.26 (s, 3H), 2.24-2.14 (m, 1H), 1.85-1.75 (m, 1H)

### Example 456: 5-[2-methyl-4-[(3S)-1-methylpyrrolidin-3-yl]oxy-pyrazol-3-yl]-N-(2-methylpyrimidin-4-yl)pyrazolo[1,5-a]pyridin-2-amine

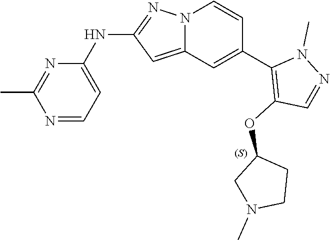

The title compound (50.1 mg, 25%) was prepared as described in Example 7, except 4-chloro-2-methylpyrimidine was used instead of 2-chloro-6-(trifluoromethyl)pyrazine. MS (ESI): mass calcd. for C₂₁H₂₄N₈O, 404.2. m/z found, 405.3 [M+H]⁺. ¹H NMR (400 MHz, DMSO-d6) δ 10.34 (s, 1H), 8.62 (d, J=7.2 Hz, 1H), 8.28 (d, J=5.6 Hz, 1H), 7.69 (d, J=1.2 Hz, 1H), 7.40 (s, 1H), 7.21-7.09 (m, 1H), 6.93 (dd, J=1.6, 7.2 Hz, 1H), 6.90 (s, 1H), 4.71-4.63 (m, 1H), 3.86 (s, 3H), 2.72-2.59 (m, 3H), 2.52-2.51 (m, 3H), 2.38-2.28 (m, 1H), 2.23 (s, 3H), 2.21-2.13 (m, 1H), 1.84-1.72 (m, 1H)

### Example 457: 5-[2-methyl-4-[(3S)-1-methylpyrrolidin-3-yl]oxy-pyrazol-3-yl]-N-[5-(trifluoromethyl)pyridazin-3-yl]pyrazolo[1,5-a]pyridin-2-amine

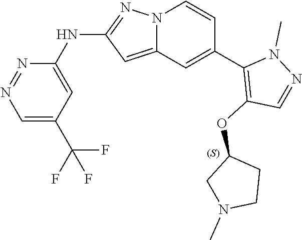

The title compound (44 mg, 20%) was prepared as described in Example 7, except 3-chloro-5-(trifluoromethyl)pyridazine was used instead of 2-chloro-6-(trifluoromethyl)pyrazine, palladium (II) acetate was used instead of BrettPhos-Pd-G3, Xantphos was used instead of Brettphos, and cesium fluoride was used instead of cesium carbonate. MS (ESI): mass calcd. for C₂₁H₂₁F₃N₈O, 458.2. m/z found, 459.2 [M+H]⁺. ¹H NMR (400 MHz, DMSO-d6) δ 10.72 (s, 1H), 9.12 (d, J=1.6 Hz, 1H), 8.67 (d, J=6.8 Hz, 1H), 7.95 (s, 1H), 7.74 (d, J=1.2 Hz, 1H), 7.40 (s, 1H), 7.01-6.91 (m, 2H), 4.72-4.61 (m, 1H), 3.86 (s, 3H), 2.69-2.58 (m, 3H), 2.33-2.26 (m, 1H), 2.21 (s, 3H), 2.19-2.13 (m, 1H), 1.82-1.72 (m, 1H) 19F NMR (376 MHz, DMSO-d6) δ −63.74 (s, 3F)

### Example 458: 5-[2-methyl-4-[(3S)-1-methylpyrrolidin-3-yl]oxy-pyrazol-3-yl]-N-[2-(trifluoromethyl)pyrimidin-4-yl]pyrazolo[1,5-a]pyridin-2-amine

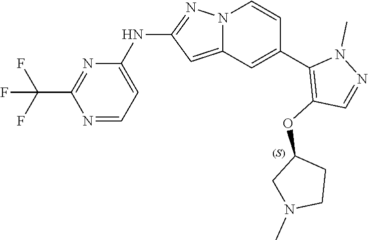

The title compound (24 mg, 11%) was prepared as described in Example 7, except 4-chloro-2-(trifluoromethyl)pyrimidine was used instead of 2-chloro-6-(trifluoromethyl)pyrazine, palladium (II) acetate was used instead of BrettPhos-Pd-G3, Xantphos was used instead of Brettphos, and cesium fluoride was used instead of cesium carbonate. MS (ESI): mass calcd. for C₂₁H₂₁F₃N₈O, 458.2. m/z found, 459.3 [M+H]⁺. ¹H NMR (400 MHz, DMSO-d6) δ 11.07 (br. s., 1H), 8.67 (d, J=7.2 Hz, 1H), 8.57 (d, J=6.0 Hz, 1H), 7.80-7.74 (m, 1H), 7.68-7.45 (m, 1H), 7.40 (s, 1H), 6.99 (dd, J=2.0, 7.2 Hz, 1H), 6.93-6.64 (m, 1H), 4.73-4.63 (m, 1H), 3.87 (s, 3H), 2.67-2.58 (m, 3H), 2.31-2.26 (m, 1H), 2.22 (s, 3H), 2.20-2.14 (m, 1H), 1.82-1.72 (m, 1H) t=80° C. ¹H NMR (400 MHz, DMSO-d6) δ 10.77 (br. s., 1H), 8.59 (d, J=7.6 Hz, 1H), 8.53 (d, J=6.0 Hz, 1H), 7.72-7.69 (m, 1H), 7.55 (d, J=5.6 Hz, 1H), 7.35 (s, 1H), 6.96 (dd, J=2.0, 7.6 Hz, 1H), 6.84 (s, 1H), 4.67-4.61 (m, 1H), 3.84 (s, 3H), 2.75-2.67 (m, 1H), 2.62-2.57 (m, 2H), 2.39-2.33 (m, 1H), 2.22 (s, 3H), 2.20-2.09 (m, 1H), 1.85-1.73 (m, 1H) 19F NMR (376 MHz, DMSO-d6) δ −69.73 (s, 3F)

### Example 459: 5-[2-methyl-4-[(3S)-1-methylpyrrolidin-3-yl]oxy-pyrazol-3-yl]-N-[6-(trifluoromethyl)pyrimidin-4-yl]pyrazolo[1,5-a]pyridin-2-amine

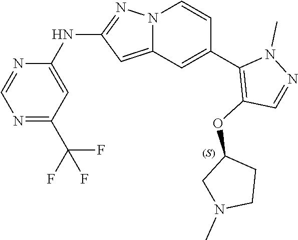

The title compound (14 mg, 6%) was prepared as described in Example 7, except 4-chloro-6-(trifluoromethyl)pyrimidine was used instead of 2-chloro-6-(trifluoromethyl)pyrazine, palladium (II) acetate was used instead of BrettPhos-Pd-G3, Xantphos was used instead of Brettphos, and cesium fluoride was used instead of cesium carbonate. MS (ESI): mass calcd. for C₂₁H₂₁F₃N₈O, 458.2. m/z found, 459.2 [M+H]⁺. ¹H NMR (400 MHz, DMSO-d6) δ 11.05 (br. s., 1H), 8.89 (s, 1H), 8.69 (d, J=6.8 Hz, 1H), 7.76 (d, J=0.8 Hz, 1H), 7.68-7.45 (m, 1H), 7.41 (s, 1H), 7.12-6.92 (m, 2H), 4.73-4.62 (m, 1H), 3.86 (s, 3H), 2.72-2.59 (m, 3H), 2.33-2.26 (m, 1H), 2.22 (s, 3H), 2.20-2.14 (m, 1H), 1.83-1.72 (m, 1H) t=80° C. ¹H NMR (400 MHz, DMSO-d6) δ 10.59 (br. s., 1H), 8.85 (s, 1H), 8.60 (d, J=6.8 Hz, 1H), 7.74-7.67 (m, 2H), 7.35 (s, 1H), 6.96 (dd, J=2.0, 7.2 Hz, 1H), 6.94 (s, 1H), 4.68-4.59 (m, 1H), 3.84 (s, 3H), 2.75-2.68 (m, 1H), 2.63-2.57 (m, 2H), 2.39-2.31 (m, 1H), 2.22 (s, 3H), 2.20-2.09 (m, 1H), 1.85-1.75 (m, 1H); 19F NMR (376 MHz, DMSO-d6) δ −69.40 (s, 3F)

### Example 460: 1-methyl-N-[5-[2-methyl-4-[(3S)-1-methylpyrrolidin-3-yl]oxy-pyrazol-3-yl]pyrazolo[1,5-a]pyridin-2-yl]pyrrolo[2,3-b]pyridin-5-amine

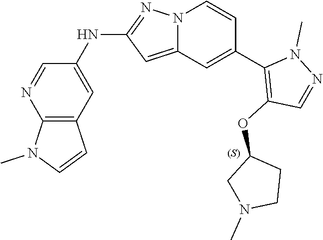

The title compound (40 mg, 27%) was prepared as described in Example 7, except 5-bromo-1-methyl-1H-pyrrolo[2,3-b]pyridine was used instead of 2-chloro-6-(trifluoromethyl)pyrazine. MS (ESI): mass calcd. for C₂₄H₂₆N₈O, 442.2. m/z found, 443.3 [M+H]⁺. ¹H NMR (400 MHz, CD3OD): 8.37 (d, J=7.2 Hz, 1H), 8.30 (d, J=2.4 Hz, 1H), 8.24 (d, J=2.4 Hz, 1H), 7.45 (d, J=0.8 Hz, 1H), 7.34 (s, 1H), 7.29 (d, J=3.2 Hz, 1H), 6.77 (dd, J=1.6, 7.2 Hz, 1H), 6.40 (d, J=3.6 Hz, 1H), 6.08 (s, 1H), 4.75-4.63 (m, 1H), 3.86 (s, 3H), 3.83 (s, 3H), 2.87-2.81 (m, 1H), 2.81-2.77 (m, 1H), 2.77-2.70 (m, 1H), 2.46-2.36 (m, 1H), 2.32 (s, 3H), 2.29-2.19 (m, 1H), 2.01-1.90 (m, 1H)

### Example 461: N-[6-(methoxymethyl)pyrimidin-4-yl]-5-[2-methyl-4-[(3S)-1-methylpyrrolidin-3-yl]oxy-pyrazol-3-yl]pyrazolo[1,5-a]pyridin-2-amine

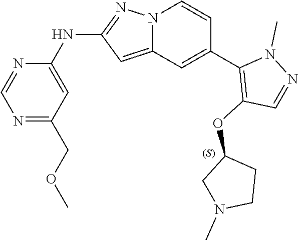

The title compound (33.2 mg, 23%) was prepared as described in Example 7, except 5-bromo-1-methyl-1H-pyrrolo[2,3-b]pyridine was used instead of 2-chloro-6-(trifluoromethyl)pyrazine. MS (ESI): mass calcd. for C₂₂H₂₆N₈O₂, 434.2. m/z found, 435.2 [M+H]⁺. ¹H NMR (400 MHz, CDCl₃) δ 8.74 (d, J=0.8 Hz, 1H), 8.39 (d, J=7.2 Hz, 1H), 7.94 (s, 1H), 7.49 (d, J=0.8 Hz, 1H), 7.36 (s, 1H), 6.88 (dd, J=2.0, 7.2 Hz, 1H), 6.73 (s, 1H), 4.67-4.60 (m, 1H), 4.51 (s, 2H), 3.92 (s, 3H), 3.53 (s, 3H), 2.82-2.71 (m, 3H), 2.46-2.38 (m, 1H), 2.36 (s, 3H), 2.29-2.18 (m, 1H), 2.05-1.95 (m, 1H)

### Example 462: N-(6-cyclopropylpyridazin-3-yl)-5-[2-methyl-4-[(3S)-1-methylpyrrolidin-3-yl]oxy-pyrazol-3-yl]pyrazolo[1,5-a]pyridin-2-amine

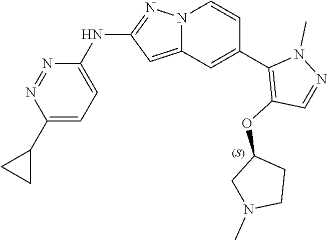

The title compound (30.2 mg, 21%) was prepared as described in Example 7, except 3-chloro-6-cyclopropylpyridazine was used instead of 2-chloro-6-(trifluoromethyl)pyrazine. MS (ESI): mass calcd. for C₂₃H₂₆N₈O, 430.2. m/z found, 431.1 [M+H]⁺. ¹H NMR (400 MHz, DMSO-d6) δ 10.02 (s, 1H), 8.59 (d, J=6.8 Hz, 1H), 7.66 (d, J=0.8 Hz, 1H), 7.53 (d, J=9.2 Hz, 1H), 7.40 (s, 1H), 7.34 (d, J=9.2 Hz, 1H), 6.92-6.87 (m, 2H), 4.70-4.63 (m, 1H), 3.85 (s, 3H), 2.70-2.58 (m, 3H), 2.35-2.26 (m, 1H), 2.21 (s, 3H), 2.20-2.10 (m, 2H), 1.84-1.70 (m, 1H), 1.04-0.97 (m, 2H), 0.97-0.91 (m, 2H)

### Example 463: N-[5-[2-methyl-4-(4-piperidyloxymethyl)pyrazol-3-yl]pyrazolo[1,5-a]pyridin-2-yl]cyclopropanecarboxamide

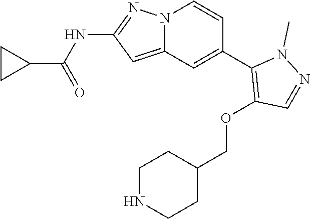

Step A. Tert-butyl 4-((5-bromo-1-methyl-1H-pyrazol-4-yl)methoxy)piperidine-1-carboxylate. To a 25 mL flask was added tert-butyl 3,3-difluoro-4-hydroxypiperidine-1-carboxylate (169.888 mg, 0.716 mmol) and DMA (1.436 mL, 0.941 g/mL, 15.515 mmol) and cooled to ° C. Then, NaH (60% dispersion in mineral oil) (47.734 mg, 1.193 mmol) The mixture was stirred for 5 min before syringe into a flask containing 5-bromo-4-(chloromethyl)-1-methyl-1H-pyrazole (80 mg, 0.247 mmol). The reaction was stirred at 80° C. in a heating block for 120 min. The crude was quenched with H₂O and then diluted with EtOAc. The organics were extracted and concentrated down in the presence of silica gel. The crude was flashed with 5-10% MeOH:DCM. The pure fractions were concentrated down to a clear oil. To the clear oil was added TFA (0.377 mL, 1.49 g/mL, 4.933 mmol) and DCM (0.948 mL, 1.326 g/mL, 14.798 mmol). The crude was evaporated in vacuo. The crude oil residue was dissolved with DCM/MeOH and concentrated down again. The crude was dissolved with DMSO and purified via ISCO prep. HPLC using 10-60% ACN-H₂O with 20 MM of NH₃OH as modifier. The fractions contained desired material was collected and concentrated down to ˜30 mL under vacuo. The mixture was transferred to a 20 mL flask, frozen, and lyophilized down to afford the product (34 mg, 38%).

Step B.N-[5-[2-methyl-4-(4-piperidyloxymethyl)pyrazol-3-yl]pyrazolo[1,5-a]pyridin-2-yl]cyclopropanecarboxamide. To a microwave vial was added a stir bar, Intermediate 3 (25 mg, 0.0764 mmol), tert-butyl 4-((5-bromo-1-methyl-1H-pyrazol-4-yl)methoxy)piperidine-1-carboxylate (34.317 mg, 0.0917 mmol), and Ruphos-G2 (2.976 mg, 0.00383 mmol). The flask was treated with 1,4-dioxane (0.255 mL, 0.3 M, 0.0764 mmol) and aqueous tribasic potassium phosphate 0.5 M in water (0.168 mL, 0.5 M, 0.084 mmol), then degassed by vacuum and back-filled with N₂ (4×). The flask was heated in the microwave for 1 h at 90 degrees C. The residue was purified via prep. HPLC using 10-90% ACN-H₂O with 20 mM of NH₄OH as modifier. The pure fraction was collected and lyophilized down to afford the product (26 mg, 67%). MS (ESI): mass calcd. for C₂₁H₂₆N₆O₂, 394.2. m/z found, 395.1 [M+H]⁺. ¹H NMR (500 MHz, Methanol-d4) δ 8.50 (dt, J=7.2, 0.9 Hz, 1H), 7.65 (dd, J=1.9, 1.0 Hz, 1H), 7.60 (s, 1H), 6.97-6.91 (m, 1H), 6.87 (dd, J=7.1, 1.9 Hz, 1H), 4.40 (s, 2H), 3.85 (s, 3H), 3.70 (tt, J=6.5, 3.2 Hz, 1H), 3.22 (ddd, J=12.8, 9.0, 3.7 Hz, 2H), 3.08-2.97 (m, 2H), 1.95 (ddt, J=12.9, 9.2, 3.6 Hz, 2H), 1.87 (tt, J=8.1, 4.6 Hz, 1H), 1.80 (dtd, J=14.1, 6.9, 3.7 Hz, 2H), 1.02-0.97 (m, 2H), 0.91 (dt, J=8.0, 3.3 Hz, 2H).

### Example 464: N-[5-[2-methyl-4-[[(3S)-1-methylpyrrolidin-3-yl]oxymethyl]pyrazol-3-yl]pyrazolo[1,5-a]pyridin-2-yl]cyclopropanecarboxamide

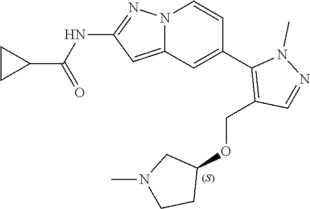

Step A. Tert-butyl (S)-3-((5-bromo-1-methyl-1H-pyrazol-4-yl)methoxy)pyrrolidine-1-carboxylate. The title compound (400 mg, 39%) was prepared as described in Example 463 Step A, except tert-butyl (S)-3-hydroxypyrrolidine-1-carboxylate was used instead of tert-butyl 3,3-difluoro-4-hydroxypiperidine-1-carboxylate.

Step B. (S)—N-(5-(1-methyl-4-((pyrrolidin-3-yloxy)methyl)-1H-pyrazol-5-yl)pyrazolo[1,5-a]pyridin-2-yl)cyclopropanecarboxamide. The title compound (28 mg, 7%) was prepared as described in Example 463: N-[5-[2-methyl-4-(4-piperidyloxymethyl)pyrazol-3-yl]pyrazolo[1,5-a]pyridin-2-yl]cyclopropanecarboxamide. Example 463 Step B, except tert-butyl (S)-3-((5-bromo-1-methyl-1H-pyrazol-4-yl)methoxy)pyrrolidine-1-carboxylate was used instead of tert-butyl 4-((5-bromo-1-methyl-1H-pyrazol-4-yl)methoxy)piperidine-1-carboxylate.

Step C. N-[5-[2-methyl-4-[[(3S)-1-methylpyrrolidin-3-yl]oxymethyl]pyrazol-3-yl]pyrazolo[1,5-a]pyridin-2-yl]cyclopropanecarboxamide. To a solution of (S)—N-(5-(1-methyl-4-((pyrrolidin-3-yloxy)methyl)-1H-pyrazol-5-yl)pyrazolo[1,5-a]pyridin-2-yl)cyclopropanecarboxamide (100 mg, 0.202 mmol) in MeOH (1.9 mL) was added H(CHO)n (0.0146 g, 0.162 mmol) at room-temperature. Then to the solution was added NaCNBH₃ (0.0248 g, 0.394 mmol) and stirred for 2 d. The crude was diluted with EtOAc and washed with H2O (1×). The organic was concentrated down and purified via preparative HPLC using 10-80% ACN-H₂O. The pure fractions were collected and lyophilized to afford a white powder (20 mg, 0.6%, 3 steps). MS (ESI): mass calcd. for C₂₁H₂₆N₆O₂, 394.2. m/z found, 395.1 [M+H]⁺. ¹H NMR (400 MHz, Methanol-d4) δ 8.50 (dt, J=7.2, 0.9 Hz, 1H), 7.66 (dd, J=2.0, 1.0 Hz, 1H), 7.58 (s, 1H), 6.96-6.92 (m, 1H), 6.89 (dd, J=7.2, 1.9 Hz, 1H), 4.29 (d, J=2.0 Hz, 2H), 4.10 (ddt, J=10.7, 7.3, 3.6 Hz, 1H), 3.85 (s, 3H), 2.75-2.63 (m, 2H), 2.58 (dd, J=10.6, 3.2 Hz, 1H), 2.50-2.39 (m, 1H), 2.31 (s, 3H), 2.06 (td, J=13.9, 7.6 Hz, 1H), 1.87 (dd, J=8.4, 4.2 Hz, 1H), 1.84-1.73 (m, 1H), 1.00 (dt, J=4.5, 3.1 Hz, 2H), 0.91 (dt, J=8.0, 3.2 Hz, 2H).

### Example 465: (*S)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide

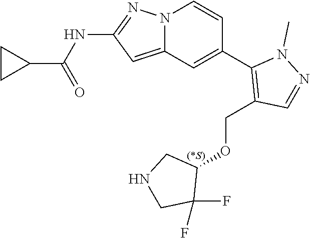 [SMILES: Cn1ncc(CO[C@H]2CNCC2(F)F)c1-c1ccn2nc(NC(=O)C3CC3)cc2c1]

Step A. Tert-butyl 4-((5-bromo-1-methyl-1H-pyrazol-4-yl)methoxy)-3,3-difluoropyrrolidine-1-carboxylate. To a 25 mL flask was added tert-butyl 3,3-difluoro-4-hydroxypyrrolidine-1-carboxylate (415.594 mg, 1.862 mmol) and DMF (7.208 mL, 0.944 g/mL, 93.091 mmol) and cooled to 0 C. Then, NaH (60% dispersion in mineral oil) (171.844 mg, 4.297 mmol). The mixture was stirred for 5 min before syringe into a flask containing 5-bromo-4-(chloromethyl)-1-methyl-1H-pyrazole (300 mg, 1.432 mmol). The reaction was heated to 80° C. for 4 h. The reaction was stopped at this point. The crude was quenched with H₂O and then diluted with EtOAc. The organics were washed 2× with H₂O and concentrated down in the presence of silica gel. The crude was flashed with 20-100% EtOAc:hex. This material (250 mg, 44%) was impure but was taken onto the next step.

Step B. N-(5-(4-(((4,4-difluoropyrrolidin-3-yl)oxy)methyl)-1-methyl-1H-pyrazol-5-yl)pyrazolo[1,5-a]pyridin-2-yl)cyclopropanecarboxamide. The title compound (170 mg, 27%) was prepared as described in Example 463: N-[5-[2-methyl-4-(4-piperidyloxymethyl)pyrazol-3-yl]pyrazolo[1,5-a]pyridin-2-yl]cyclopropanecarboxamide. Example 463 Step B, except tert-butyl 4-((5-bromo-1-methyl-1H-pyrazol-4-yl)methoxy)-3,3-difluoropyrrolidine-1-carboxylate was used instead of tert-butyl 4-((5-bromo-1-methyl-1H-pyrazol-4-yl)methoxy)piperidine-1-carboxylate.

Step C. (*S)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide. The title compound (51 mg, 25%) was prepared as described in Example 464 Step C, except N-(5-(4-(((4,4-difluoropyrrolidin-3-yl)oxy)methyl)-1-methyl-1H-pyrazol-5-yl)pyrazolo[1,5-a]pyridin-2-yl)cyclopropanecarboxamide was used instead of (S)—N-(5-(1-methyl-4-((pyrrolidin-3-yloxy)methyl)-1H-pyrazol-5-yl)pyrazolo[1,5-a]pyridin-2-yl)cyclopropanecarboxamide. The material was further purified via SFC separation using AD-H (2×25 cm) 40% IPA (0.1% DEA)/CO₂, 100 bar 50 mL/min, 220 nm inj vol.: 1 mL, 10 mg/mL ethanol. The title compound was second to elute. MS (ESI): mass calcd. for C₂₀H₂₂F₂N₆O₂, 416.2. m/z found, 417.2 [M+H]⁺. ¹H NMR (500 MHz, Methanol-d4) δ 8.48 (dt, J=7.1, 1.0 Hz, 1H), 7.65 (dd, J=2.0, 0.9 Hz, 1H), 7.59 (s, 1H), 6.93 (s, 1H), 6.88 (dd, J=7.1, 1.9 Hz, 1H), 4.55 (d, J=11.4 Hz, 1H), 4.44 (d, J=11.4 Hz, 1H), 3.98-3.89 (m, 1H), 3.85 (s, 3H), 3.28-3.20 (m, 1H), 3.20-2.98 (m, 2H), 2.82 (ddd, J=12.5, 5.2, 1.6 Hz, 1H), 1.87 (tt, J=8.0, 4.5 Hz, 1H), 0.99 (dt, J=4.6, 3.1 Hz, 2H), 0.93-0.85 (m, 2H).

### Example 466: N-[5-[4-[[(3R,4R)-4-fluoropyrrolidin-3-yl]oxymethyl]-2-methyl-pyrazol-3-yl]pyrazolo[1,5-a]pyridin-2-yl]cyclopropanecarboxamide

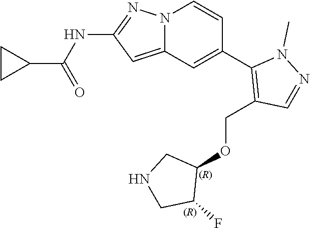

Step A. Tert-butyl (3R,4R)-3-((5-bromo-1-methyl-1H-pyrazol-4-yl)methoxy)-4-fluoropyrrolidine-1-carboxylate. The title compound (400 mg, 39%) was prepared as described in Example 463 Step A, except tert-butyl (3R,4R)-3-fluoro-4-hydroxypyrrolidine-1-carboxylate was used instead of tert-butyl 3,3-difluoro-4-hydroxypiperidine-1-carboxylate.

Step B. N-[5-[4-[[(3R,4R)-4-fluoropyrrolidin-3-yl]oxymethyl]-2-methyl-pyrazol-3-yl]pyrazolo[1,5-a]pyridin-2-yl]cyclopropanecarboxamide. The title compound (35 mg, 16.6%) was prepared as described in Example 463 Step B, except tert-butyl (3R,4R)-3-((5-bromo-1-methyl-1H-pyrazol-4-yl)methoxy)-4-fluoropyrrolidine-1-carboxylate was used instead of tert-butyl 4-((5-bromo-1-methyl-1H-pyrazol-4-yl)methoxy)piperidine-1-carboxylate. MS (ESI): mass calcd. for C₂₀H₂₃FN₆O₂, 398.2. m/z found, 399.2 [M+H]⁺. ¹H NMR (500 MHz, Methanol-d4) δ 8.50 (dt, J=7.1, 1.0 Hz, 1H), 7.62 (dd, J=2.0, 1.0 Hz, 1H), 7.59 (s, 1H), 6.95 (s, 1H), 6.86 (dd, J=7.1, 1.9 Hz, 1H), 5.03-4.89 (m, 1H), 4.42 (d, J=1.9 Hz, 2H), 4.07-3.99 (m, 1H), 3.84 (s, 3H), 3.14-2.94 (m, 3H), 2.77 (dd, J=12.5, 2.9 Hz, 1H), 1.87 (tt, J=8.0, 4.5 Hz, 1H), 0.99 (dt, J=4.6, 3.2 Hz, 2H), 0.90 (dt, J=8.0, 3.3 Hz, 2H).

### Example 467: N-[5-[4-[[(3R,4R)-4-fluoro-1-methyl-pyrrolidin-3-yl]oxymethyl]-2-methyl-pyrazol-3-yl]pyrazolo[1,5-a]pyridin-2-yl]cyclopropanecarboxamide

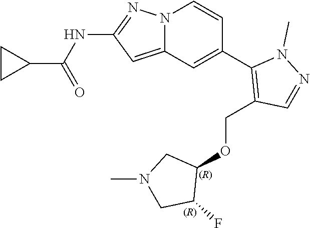

The title compound (44 mg, 39%) was prepared as described in Example 464 Step C, except N-[5-[4-[[(3R,4R)-4-fluoropyrrolidin-3-yl]oxymethyl]-2-methyl-pyrazol-3-yl]pyrazolo[1,5-a]pyridin-2-yl]cyclopropanecarboxamide (from Example 86) was used instead of (S)—N-(5-(1-methyl-4-((pyrrolidin-3-yloxy)methyl)-1H-pyrazol-5-yl)pyrazolo[1,5-a]pyridin-2-yl)cyclopropanecarboxamide. MS (ESI): mass calcd. for C₂₁H₂₅FN₆O₂, 412.2. m/z found, 413.2 [M+H]⁺. ¹H NMR (500 MHz, Methanol-d4) δ 8.49 (dt, J=7.2, 0.9 Hz, 1H), 7.63 (dd, J=1.9, 1.0 Hz, 1H), 7.59 (s, 1H), 6.94 (s, 1H), 6.86 (dd, J=7.1, 1.9 Hz, 1H), 5.01-4.85 (m, 1H), 4.40 (q, J=11.6 Hz, 2H), 4.15-4.03 (m, 1H), 3.85 (s, 3H), 3.07 (dd, J=10.3, 6.5 Hz, 1H), 2.92-2.80 (m, 1H), 2.70 (ddd, J=29.8, 11.7, 5.3 Hz, 1H), 2.30 (s, 4H), 1.87 (tt, J=8.1, 4.5 Hz, 1H), 0.99 (dt, J=4.6, 3.2 Hz, 2H), 0.93-0.85 (m, 2H).

### Example 468: N-[5-[4-[(3-ethyl-1-methyl-azetidin-3-yl)oxymethyl]-2-methyl-pyrazol-3-yl]pyrazolo[1,5-a]pyridin-2-yl]cyclopropanecarboxamide

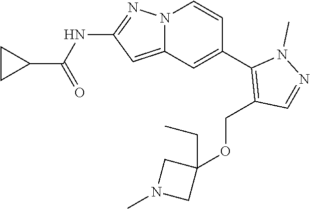 [SMILES: CCC1(OCc2cnn(C)c2-c2ccn3nc(NC(=O)C4CC4)cc3c2)CN(C)C1]

Step A. Tert-butyl 3-((5-bromo-1-methyl-1H-pyrazol-4-yl)methoxy)-3-ethylazetidine-1-carboxylate. The title compound (460 mg, 86%) was prepared as described in Example 463 Step A, except tert-butyl 3-ethyl-3-hydroxyazetidine-1-carboxylate was used instead of tert-butyl 3,3-difluoro-4-hydroxypiperidine-1-carboxylate.

Step B. N-(5-(4-(((3-ethylazetidin-3-yl)oxy)methyl)-1-methyl-1H-pyrazol-5-yl)pyrazolo[1,5-a]pyridin-2-yl)cyclopropanecarboxamide. The title compound (210 mg, 43%) was prepared as described in Example 463 Step B, except tert-butyl 3-((5-bromo-1-methyl-1H-pyrazol-4-yl)methoxy)-3-ethylazetidine-1-carboxylate was used instead of tert-butyl 4-((5-bromo-1-methyl-1H-pyrazol-4-yl)methoxy)piperidine-1-carboxylate.

Step C. N-[5-[4-[(3-ethyl-1-methyl-azetidin-3-yl)oxymethyl]-2-methyl-pyrazol-3-yl]pyrazolo[1,5-a]pyridin-2-yl]cyclopropanecarboxamide. The title compound was prepared as described in Example 464 Step C, except N-(5-(4-(((3-ethylazetidin-3-yl)oxy)methyl)-1-methyl-1H-pyrazol-5-yl)pyrazolo[1,5-a]pyridin-2-yl)cyclopropanecarboxamide was used instead of (S)—N-(5-(1-methyl-4-((pyrrolidin-3-yloxy)methyl)-1H-pyrazol-5-yl)pyrazolo[1,5-a]pyridin-2-yl)cyclopropanecarboxamide. MS (ESI): mass calcd. for C₂₂H₂₈N₆O₂, 408.2. m/z found, 409.2 [M+H]⁺. ¹H NMR (400 MHz, Methanol-d4) δ 8.51 (dt, J=7.1, 1.0 Hz, 1H), 7.67 (dd, J=2.0, 1.0 Hz, 1H), 7.59 (s, 1H), 6.95 (s, 1H), 6.89 (dd, J=7.1, 1.9 Hz, 1H), 4.18 (s, 2H), 3.85 (s, 3H), 3.29-3.21 (m, 2H), 3.06-2.95 (m, 2H), 2.30 (s, 3H), 1.94-1.76 (m, 3H), 1.00 (dt, J=4.6, 3.1 Hz, 2H), 0.96-0.86 (m, 2H), 0.80 (t, J=7.3 Hz, 3H).

### Example 469: (rac-trans)-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine

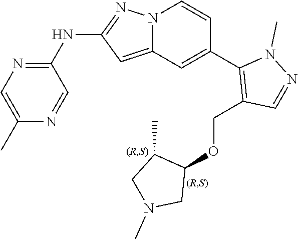

Step A. Tert-butyl (3RS,4RS)-3-((5-bromo-1-methyl-1H-pyrazol-4-yl)methoxy)-4-methylpyrrolidine-1-carboxylate. The title compound (17.3 mg, 37%) was prepared as described in Example 463 Step A, except trans-tert-butyl-3-hydroxy-4-methylpyrrolidine-1-carboxylate was used instead of tert-butyl 3,3-difluoro-4-hydroxypiperidine-1-carboxylate.

Step B. 5-bromo-4-((((3R,4S)-1,4-dimethylpyrrolidin-3-yl)oxy)methyl)-1-methyl-1H-pyrazole. The title compound (75 mg, 43%) was prepared as described in Example 464 Step C, except tert-butyl (3RS,4RS)-3-((5-bromo-1-methyl-1H-pyrazol-4-yl)methoxy)-4-methylpyrrolidine-1-carboxylate was used instead of (S)—N-(5-(1-methyl-4-((pyrrolidin-3-yloxy)methyl)-1H-pyrazol-5-yl)pyrazolo[1,5-a]pyridin-2-yl)cyclopropanecarboxamide.

Step C. 5-(4-((((3R,4S)-1,4-dimethylpyrrolidin-3-yl)oxy)methyl)-1-methyl-1H-pyrazol-5-yl)pyrazolo[1,5-a]pyridin-2-amine. The title compound (20 mg, 46%) was prepared as described in Example 1 Step D, except Intermediate 5 was used instead of Intermediate 3 and 5-bromo-4-((((3R,4S)-1,4-dimethylpyrrolidin-3-yl)oxy)methyl)-1-methyl-1H-pyrazole was used instead of tert-butyl (2S,4S)-4-((5-bromo-1-methyl-1H-pyrazol-4-yl)oxy)-2-methylpyrrolidine-1-carboxylate.

Step D. 5-(4-((((3R,4S)-1,4-dimethylpyrrolidin-3-yl)oxy)methyl)-1-methyl-1H-pyrazol-5-yl)-N-(5-methylpyrazin-2-yl)pyrazolo[1,5-a]pyridin-2-amine. The title compound (3.4 mg, 14%) was prepared as described in Example 2 Step A, except 5-(4-((((3R,4S)-1,4-dimethylpyrrolidin-3-yl)oxy)methyl)-1-methyl-1H-pyrazol-5-yl)pyrazolo[1,5-a]pyridin-2-amine was used instead of Intermediate 6 and 2-chloro-5-methylpyrazine was used instead of 4-chloro-2,6-dimethylpyrimidine. MS (ESI): mass calcd. for C₂₃H₂₈N₈O, 432.2. m/z found, 433.2 [M+H]⁺. ¹H NMR (400 MHz, Methanol-d4) δ 8.47 (d, J=1.5 Hz, 1H), 8.41 (dd, J=7.1, 1.0 Hz, 1H), 8.04 (d, J=1.4 Hz, 1H), 7.56-7.52 (m, 1H), 7.49 (s, 1H), 6.81-6.68 (m, 2H), 4.22 (s, 2H), 3.77 (s, 3H), 3.56 (s, 2H), 3.53 (dd, J=6.5, 3.3 Hz, 1H), 2.91-2.77 (m, 1H), 2.65 (dd, J=10.6, 3.1 Hz, 1H), 2.47 (dd, J=10.6, 6.3 Hz, 1H), 2.35 (s, 3H), 2.19 (s, 3H), 2.14-2.00 (m, 1H), 1.92-1.82 (m, 1H), 0.95 (d, J=7.1 Hz, 3H).

### Example 470: N-[5-[2-(difluoromethyl)-4-[(3S)-1-methylpyrrolidin-3-yl]oxy-pyrazol-3-yl]pyrazolo[1,5-a]pyridin-2-yl]cyclopropanecarboxamide

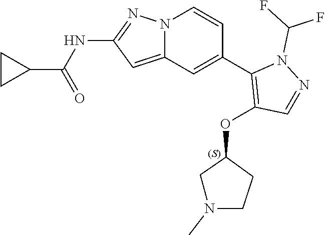

Step A. 4-iodo-1-((2-(trimethylsilyl)ethoxy)methyl)-1H-pyrazole. NaH (4.9 g, 0.21 mol in mineral oil) was added to a 0° C. (ice/water) solution consisting of 4-iodo-1H-pyrazole (20 g, 0.10 mol) and THF (200 mL). The reaction mixture was stirred at room-temperature for 2 h. SEM-Cl (18.9 mg, 0.113 mol) was added to the mixture at room-temperature. The reaction mixture was stirred overnight at room-temperature. The reaction mixture was concentrated under reduced pressure to give the crude product, which was purified by FCC (eluent: petroleum ether:ethyl acetate=1:0 to 8:2) to afford product (26.8 g, 80%) as a white oil.

Step B. 4-iodo-1-((2-(trimethylsilyl)ethoxy)methyl)-1H-pyrazole. 16 g of 4-iodo-1-((2-(trimethylsilyl)ethoxy)methyl)-1H-pyrazole was carried out in 8 parallel reactions.

A mixture consisting of 4-iodo-1-((2-(trimethylsilyl)ethoxy)methyl)-1H-pyrazole (2.00 g, 6.17 mmol), (S)-1-methylpyrrolidin-3-ol (1.87 g, 18.5 mmol), CuI (0.294 g, 1.54 mmol), CS₂CO₃ (6.03 g, 18.5 mmol), L-proline (0.355 g, 3.08 mmol) and toluene (10 mL) was purged with Ar for 2 min and stirred overnight at 140° C. The reaction mixture were concentrated to dryness under reduced pressure to give the crude product. The reaction mixture was concentrated to dryness under reduced pressure to give the crude product, which was purified by FCC (eluent: petroleum ether:ethyl acetate=100:0 to 70:30) to afford the title compound (10 g, 62%) as a light yellow oil.

Step C. Tert-butyl (S)-3-((1-((2-(trimethylsilyl)ethoxy)methyl)-1H-pyrazol-4-yl)oxy)pyrrolidine-1-carboxylate. A mixture consisting of 4-iodo-1-((2-(trimethylsilyl)ethoxy)methyl)-1H-pyrazole (2.20 g, 6.79 mmol), (S)-1-methylpyrrolidin-3-ol (1.91 g, 10.2 mmol), CuI (323 mg, 1.70 mmol), Cs₂CO₃ (6.63 g, 20.4 mmol), Me₄-phenanthroline (241 mg, 1.02 mmol) and toluene (20 mL) was purged with Ar for 2 min and stirred overnight at 120° C. The crude product was concentrated under reduced pressure to give the desired product which was purified by FCC (petroleum ether:ethyl acetate=1:0 to 2:1) to afford the title compound as a yellow solid (4.74 g, 61%).

Step D. Tert-butyl (S)-3-((5-bromo-1-((2-(trimethylsilyl)ethoxy)methyl)-1H-pyrazol-4-yl)oxy)pyrrolidine-1-carboxylate. A solution of (S)-tert-butyl 3-((1-((2-(trimethylsilyl)ethoxy)methyl)-1H-pyrazol-4-yl)oxy)pyrrolidine-1-carboxylate (4.74 g, 12.4 mmol) in CH₃CN (40 mL) was added NBS (2.20 g, 12.4 mmol) and stirring at room temperature for 2 h. The solution was concentrated under reduced pressure to give the crude product which was purified by FCC (petroleum ether:ethyl acetate=1:0 to 3:1) to afford the desired product as a light yellow oil (7 g, 95%).

Step E. Tert-butyl (S)-3-((5-bromo-1H-pyrazol-4-yl)oxy)pyrrolidine-1-carboxylate A solution consisting of (S)-tert-butyl 3-((5-bromo-1-((2-(trimethylsilyl)ethoxy)methyl)-1H-pyrazol-4-yl)oxy)pyrrolidine-1-carboxylate (6.90 g, 14.9 mmol), TBAF (149 mL, 149 mmol), was stirred for 12 h at 50° C. The reaction mixture was concentrated under reduced pressure to give the crude product which was purified by FCC (petroleum ether:ethyl acetate=1:0 to 0:1) to afford the desired product as a light yellow oil (4 g, 81%).

Step F. Tert-butyl (S)-3-((5-bromo-1-(difluoromethyl)-1H-pyrazol-4-yl)oxy)pyrrolidine-1-carboxylate. A mixture of (S)-tert-butyl 3-((5-bromo-1H-pyrazol-4-yl)oxy)pyrrolidine-1-carboxylate (400 mg, 1.20 mmol) and CH₂Cl₂ (15 mL) at 0° C. was added an aqueous potassium hydroxide (405 mg, 7.23 mmol) with vigorous stirring. Then a solution of (bromodifluoromethyl)trimethylsilane (489 mg, 2.41 mmol) in CH₂Cl₂ (15 mL) was added into the mixture at 0° C. After being stirred at 0° C. for 30 min. The crude product was concentrated under reduced pressure to give the desired product which was purified by FCC (petroleum ether: ethyl acetate=1:0 to 3:1) to afford the title compound as a yellow solid (283 mg, 61%).

Step G. Tert-butyl (S)-3-((5-(2-(cyclopropanecarboxamido)pyrazolo[1,5-a]pyridin-5-yl)-1-(difluoromethyl)-1H-pyrazol-4-yl)oxy)pyrrolidine-1-carboxylate. A mixture consisting of (S)-tert-butyl 3-((3-bromo-1-(difluoromethyl)-1H-pyrazol-4-yl)oxy)pyrrolidine-1-carboxylate (257 mg, 0.336 mmol), N-(5-(4,4,5,5-tetramethyl-1,3,2-dioxaborolan-2-yl)pyrazolo[1,5-a]pyridin-2-yl)cyclopropanecarboxamide (220 mg, 0.672 mmol), Pd(dtbpf)Cl₂ (22 mg, 0.034 mmol), CsF (306 mg, 2.02 mmol), and 1,4-dioxane/H₂O (3 mL, 4:1) under N2 atmosphere was stirred 1 h at 80° C.

The reaction mixture was concentrated under reduced pressure to give the crude product which was purified by FCC (petroleum ether:ethyl acetate=1:2) to afford the title compound as a white solid (340 mg, mixture). The solid was further purified by preparative HPLC using a Xtimate C18 250 mm×80 mm×10 μm column (eluent: 43% to 73% (v/v) CH₃CN and H₂O with 0.04% NH₃+10 mM NH₄HCO₃) to afford pure product. The product was suspended in water (10 mL), the mixture frozen using dry ice/ethanol, and then lyophilized to dryness to afford the title compound (60 mg, 18%) as a white solid (NOE 2D confirmed).

Step H. (S)—N-(5-(1-(difluoromethyl)-4-(pyrrolidin-3-yloxy)-1H-pyrazol-5-yl)pyrazolo[1,5-a]pyridin-2-yl)cyclopropanecarboxamide. TFA (0.384 mL, 5.19 mmol) was added to a solution consisting of (S)-tert-butyl 3-((5-(2-(cyclopropanecarboxamido)pyrazolo[1,5-a]pyridin-5-yl)-1-(difluoromethyl)-1H-pyrazol-4-yl)oxy)pyrrolidine-1-carboxylate (64.0 mg, 0.127 mmol) under 0° C. and the solution was stirred for 1 h, then the solution was warmed to room-temperature and stirred for 1 h. The reaction mixture was concentrated under reduced pressure to give the crude product, and the saturated NaHCO₃ aqueous solution until pH=7˜8, the crude solution was dried under high vacuum to afford the title compound as a brown solid (80 mg, crude).

Step I. N-[5-[2-(difluoromethyl)-4-[(3S)-1-methylpyrrolidin-3-yl]oxy-pyrazol-3-yl]pyrazolo[1,5-a]pyridin-2-yl]cyclopropanecarboxamide. A mixture consisting of (S)—N-(5-(1-(difluoromethyl)-4-(pyrrolidin-3-yloxy)-1H-pyrazol-5-yl)pyrazolo[1,5-a]pyridin-2-yl)cyclopropanecarboxamide (60 mg, 0.149 mmol), paraformaldehyde (26.9 mg, 0.298 mmol), and MeOH (6 mL) was stirred for 10 min followed by addition of NaBH₃CN (37.5 mg, 0.596 mmol). The crude product was further purified by preparative HPLC with Boston Green ODS 150×30 mm×5 um, column (eluent: 25% to 55% (v/v) CH₃CN and H₂O with 0.04% NH₃H₂O+10 mM NH₄HCO₃) to afford pure product. The product was suspended in water (10 mL), the mixture frozen using dry ice/EtOH, and then lyophilized to dryness to afford the title compound as a white solid (20.7 mg, 33%). MS (ESI): mass calcd. for C₂₀H₂₂F₂N₆O₂, 416.2. m/z found, 417.2 [M+H]⁺. ¹H NMR (400 MHz, DMSO-d6) δ 11.14 (s, 1H), 8.63 (d, J=5.6 Hz, 1H), 7.90 (s, 1H), 7.87-7.52 (m, 2H), 6.94 (s, 1H), 6.84 (d, J=5.2 Hz, 1H), 4.88-4.73 (m, 1H), 2.66-2.57 (m, 3H), 2.35-2.24 (m, 2H), 2.21 (s, 3H), 1.99-1.88 (m, 1H), 1.81-1.70 (m, 1H), 0.93-0.76 (m, 4H); 19F NMR (376 MHz, DMSO-d6) δ −92.77 (dd, J=17.67, 57.90 Hz, 2F)

### Example 471: N-[6-[2-methyl-4-[(3S)-pyrrolidin-3-yl]oxy-pyrazol-3-yl]imidazo[1,2-a]pyridin-2-yl]cyclopropanecarboxamide

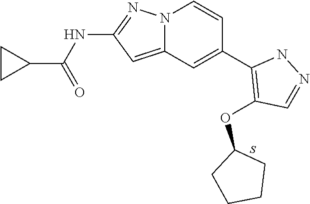

The title compound was prepared (197 mg, 62%) in the similar manner of Example 4 while 6-bromoimidazo[1,2-a]pyridin-2-amine was used instead of 5-bromopyrazolo[1,5-a]pyridin-2-amine. MS (ESI): mass calcd. for C₁₉H₂₂N₆O₂, 366.2. m/z found, 367.1 [M+H]⁺. ¹H NMR (500 MHz, Methanol-d4) δ 8.49 (t, J=1.4 Hz, 1H), 8.03 (s, 1H), 7.50 (d, J=9.2 Hz, 1H), 7.40 (s, 1H), 7.38-7.29 (m, 1H), 4.81 (t, J=4.4 Hz, 1H), 3.74 (s, 3H), 3.49-3.40 (m, 1H), 3.39-3.28 (m, 2H), 3.19-3.13 (m, 1H), 2.24-2.15 (m, 1H), 2.13-2.01 (m, 1H), 1.88-1.76 (m, 1H), 0.94-0.88 (m, 2H), 0.88-0.77 (m, 2H).

### Example 472: N-[6-[2-methyl-4-[(3S)-1-methylpyrrolidin-3-yl]oxy-pyrazol-3-yl]imidazo[1,2-a]pyridin-2-yl]cyclopropanecarboxamide

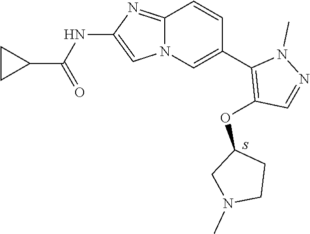

The title compound was prepared (52 mg, 40%) in the similar manner of Example 1 Step F, while N-[6-[2-methyl-4-[(3S)-pyrrolidin-3-yl]oxy-pyrazol-3-yl]imidazo[1,2-a]pyridin-2-yl]cyclopropanecarboxamide (from Example 471) was used instead of N-(5-(1-methyl-4-(((3S,5S)-5-methylpyrrolidin-3-yl)oxy)-1H-pyrazol-5-yl)pyrazolo[1,5-a]pyridin-2-yl)cyclopropanecarboxamide. MS (ESI): mass calcd. for C₂₀H₂₄N₆O₂, 380.2. m/z found, 381.1 [M+H]⁺. ¹H NMR (500 MHz, Methanol-d4) δ 8.56-8.40 (m, 1H), 8.01 (s, 1H), 7.43 (d, J=9.2 Hz, 1H), 7.32-7.20 (m, 2H), 4.68-4.58 (m, 1H), 3.73 (s, 3H), 2.91-2.74 (m, 2H), 2.70-2.62 (m, 1H), 2.45-2.31 (m, 1H), 2.28 (s, 3H), 2.21-2.11 (m, 1H), 1.96-1.81 (m, 2H), 0.95-0.86 (m, 2H), 0.86-0.75 (m, 2H).

### Example 473: cis-N-[5-[2-methyl-4-[(1-methylazetidin-2-yl)methoxy]pyrazol-3-yl]pyrazolo[1,5-a]pyridin-2-yl]-2-phenyl-cyclopropanecarboxamide

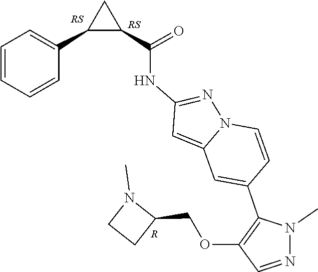

The title compound (108 mg, 46.6%). was prepared in the similar manner of Example 156 while cis-2-phenyl-1-cyclopropanecarboxylic acid was used instead of trans-2-phenyl-1-cyclopropanecarboxylic acid. MS (ESI): mass calcd. for C₂₆H₂₈N₆O₂, 456.2. m/z found, 457.2 [M+H]⁺. ¹H NMR (500 MHz, Methanol-d4) δ 8.34-8.20 (m, 1H), 7.40 (dd, J=2.0, 0.9 Hz, 1H), 7.29 (s, 1H), 7.16 (dd, J=8.0, 1.4 Hz, 2H), 7.11-7.03 (m, 2H), 7.02-6.96 (m, 1H), 6.78 (dd, J=7.2, 2.0 Hz, 1H), 6.49 (d, J=0.9 Hz, 1H), 3.94-3.79 (m, 2H), 3.70 (s, 3H), 3.38-3.27 (m, 1H), 3.24-3.22 (m, 1H), 2.82-2.71 (m, 1H), 2.57-2.44 (m, 1H), 2.26-2.18 (m, 1H), 2.16 (s, 3H), 1.99-1.77 (m, 2H), 1.73-1.61 (m, 1H), 1.31-1.20 (m, 1H).

### Example 474: (1*R,2*S)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide

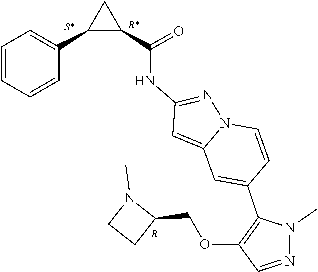

The title compound (28 mg, 12%). was prepared in the similar manner of Example 156 while cis-2-phenyl-1-cyclopropanecarboxylic acid was used instead of trans-2-phenyl-1-cyclopropanecarboxylic acid and followed by SFC chiral separation. MS (ESI): mass calcd. for C₂₆H₂₈N₆O₂, 456.2. m/z found, 457.2 [M+H]⁺. ¹H NMR (500 MHz, Methanol-d4) δ 8.34-8.20 (m, 1H), 7.40 (dd, J=2.0, 0.9 Hz, 1H), 7.29 (s, 1H), 7.16 (dd, J=8.0, 1.4 Hz, 2H), 7.11-7.03 (m, 2H), 7.02-6.96 (m, 1H), 6.78 (dd, J=7.2, 2.0 Hz, 1H), 6.49 (d, J=0.9 Hz, 1H), 3.94-3.79 (m, 2H), 3.70 (s, 3H), 3.38-3.27 (m, 1H), 3.24-3.22 (m, 1H), 2.82-2.71 (m, 1H), 2.57-2.44 (m, 1H), 2.26-2.18 (m, 1H), 2.16 (s, 3H), 1.99-1.77 (m, 2H), 1.73-1.61 (m, 1H), 1.31-1.20 (m, 1H).

### Example 475: cis-2-cyano-N-[5-[2-methyl-4-[(1-methylazetidin-2-yl)methoxy]pyrazol-3-yl]pyrazolo[1,5-a]pyridin-2-yl]cyclopropanecarboxamide

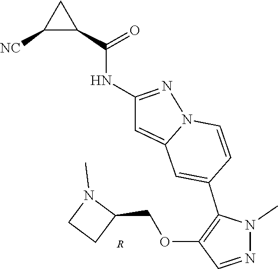

The title compound was prepared (3 mg, 6%) by the similar method of Example 26 Step C by using cis-2-cyanocyclopropane-1-carboxylic acid instead of 1-methyl-1H-pyrazole-4-carboxylic acid. MS (ESI): mass calcd. for C₂₁H₂₃N₇O₂, 405.2. m/z found, 406.1 [M+H]⁺. ¹H NMR (500 MHz, Methanol-d4) δ 8.56-8.44 (m, 1H), 7.73-7.66 (m, 1H), 7.47 (s, 1H), 7.40-7.31 (m, 1H), 7.04-6.95 (m, 2H), 4.13-3.98 (m, 2H), 3.91 (s, 3H), 3.54-3.42 (m, 1H), 3.42-3.37 (m, 1H), 2.98-2.85 (m, 1H), 2.50-2.41 (m, 1H), 2.38-2.32 (m, 3H), 2.21-1.95 (m, 3H), 1.74-1.64 (m, 1H), 1.57-1.44 (m, 1H).

### Example 476: N-(2,6-dimethylpyrimidin-4-yl)-6-[4-[[(2R)-1-ethylazetidin-2-yl]methoxy]-2-methyl-pyrazol-3-yl]imidazo[1,2-a]pyridin-2-amine

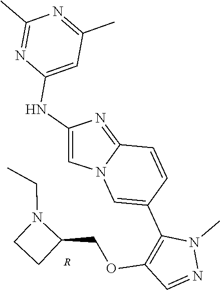

The title compound was prepared (58 mg, 36%) by the similar method of Example 4 while 6-bromoimidazo[1,2-a]pyridin-2-amine was used instead of 5-bromopyrazolo[1,5-a]pyridin-2-amine. MS (ESI): mass calcd. for C₂₃H₂₈N₈O, 432.2. m/z found, [M+H]⁺. ¹H NMR (400 MHz, Methanol-d4) δ 8.52 (t, J=1.4 Hz, 1H), 8.29 (s, 1H), 7.46-7.40 (m, 1H), 7.36 (s, 1H), 7.32-7.26 (m, 1H), 6.58 (s, 1H), 3.94-3.87 (m, 2H), 3.76 (s, 3H), 3.44-3.34 (m, 1H), 3.31-3.24 (m, 1H), 2.82-2.70 (m, 1H), 2.60-2.55 (m, 1H), 2.48 (s, 3H), 2.33-2.21 (m, 4H), 1.98-1.82 (m, 2H), 0.80 (t, J=7.3 Hz, 3H).

### Example 477: 5-[2-(difluoromethyl)-4-[[(2R)-1-ethylazetidin-2-yl]methoxy]pyrazol-3-yl]-N-(2-methylpyrimidin-4-yl)pyrazolo[1,5-a]pyridin-2-amine

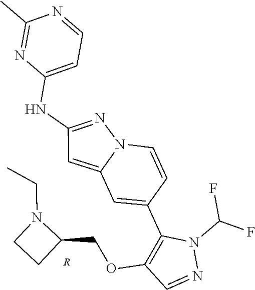

The title compound (79.4 mg, 3 steps, 30%) was prepared as described in Example 33 Step A followed by a subsequent reductive amination as described in Example 18, except 4-chloro-2-methylpyrimidine was used instead of 6-chloro-2,4-dimethylpyridazin-3(2H)-one in Step A. MS (ESI): mass calcd. for C₂₂H₂₄F₂N₈O, 454.2. m/z found, 455.2 [M+H]⁺. ¹H NMR (400 MHz, DMSO-d6): 10.39 (s, 1H), 8.67 (d, J=7.2 Hz, 1H), 8.29 (d, J=6.0 Hz, 1H), 7.96 (s, 1H), 7.90-7.59 (m, 2H), 7.15 (s, 1H), 6.95 (br. s., 1H), 6.84 (dd, J=2.0, 7.2 Hz, 1H), 4.05-3.98 (m, 2H), 3.31-3.20 (m, 2H), 2.68-2.61 (m, 1H), 2.59-2.53 (m, 1H), 2.26-2.16 (m, 1H), 1.98-1.89 (m, 1H), 1.89-1.77 (m, 1H), 0.80 (t, J=7.2 Hz, 3H) 1H NMR (400 MHz, CD3OD): 8.48 (d, J=7.2 Hz, 1H), 8.21 (d, J=6.0 Hz, 1H), 7.79 (s, 1H), 7.67 (d, J=0.4 Hz, 1H), 7.45 (t, J=58.0 Hz, 1H), 7.15 (d, J=4.4 Hz, 1H), 6.96 (s, 1H), 6.91 (dd, J=1.6, 7.2 Hz, 1H), 4.15-4.02 (m, 2H), 3.54-3.46 (m, 1H), 3.40-3.34 (m, 1H), 2.90-2.82 (m, 1H), 2.74-2.64 (m, 1H), 2.56 (s, 3H), 2.43-2.32 (m, 1H), 2.12-2.04 (m, 1H), 2.03-1.93 (m, 1H), 0.91 (t, J=7.2 Hz, 3H) 19F NMR (376 MHz, CD3OD): −94.29-−94.41 (m, 2F)

### Example 478: 5-[2-(difluoromethyl)-4-[[(2R)-1-ethylazetidin-2-yl]methoxy]pyrazol-3-yl]-N-(6-methylpyrimidin-4-yl)pyrazolo[1,5-a]pyridin-2-amine

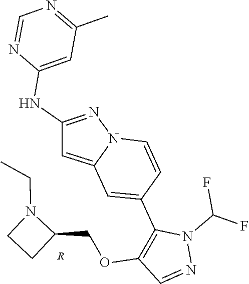

The title compound (81.3 mg, 3 steps, 36%) was prepared as described in Example 33 Step A followed by a subsequent reductive amination as described in Example 18, except 4-chloro-6-methylpyrimidine was used instead of 6-chloro-2,4-dimethylpyridazin-3(2H)-one in Step A. MS (ESI): mass calcd. for C₂₂H₂₄F₂N₈O, 454.2. m/z found, 455.3 [M+H]⁺. ¹H NMR (400 MHz, DMSO-d6): 10.36 (s, 1H), 8.67 (d, J=7.2 Hz, 1H), 8.59 (s, 1H), 7.95 (s, 1H), 7.90-7.57 (m, 2H), 7.14 (s, 1H), 6.95 (s, 1H), 6.84 (dd, J=2.0, 7.2 Hz, 1H), 4.03 (d, J=5.6 Hz, 2H), 3.30-3.20 (m, 2H), 2.71-2.61 (m, 1H), 2.58-2.52 (m, 1H), 2.35 (s, 3H), 2.28-2.16 (m, 1H), 1.99-1.89 (m, 1H), 1.89-1.77 (m, 1H), 0.79 (t, J=7.2 Hz, 3H) 19F NMR (376 MHz, DMSO-d6): −93.29-−92.05 (m, 2F)

### Example 479: 5-[2-(difluoromethyl)-4-[[(2R)-1-ethylazetidin-2-yl]methoxy]pyrazol-3-yl]-N-(2,6-dimethylpyrimidin-4-yl)pyrazolo[1,5-a]pyridin-2-amine

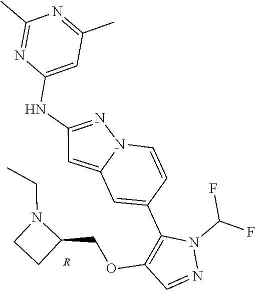

The title compound (63.9 mg, 3 steps, 43%) was prepared as described in Example 33 Step A followed by a subsequent reductive amination as described in Example 18, except 4-chloro-2,6-dimethylpyrimidine was used instead of 6-chloro-2,4-dimethylpyridazin-3(2H)-one in Step A. MS (ESI): mass calcd. for C₂₃H₂₆F₂N₈O, 468.2. m/z found, 469.2 [M+H]⁺. ¹H NMR (400 MHz, CDCl₃): 8.42 (d, J=7.2 Hz, 1H), 7.71 (s, 1H), 7.66-7.58 (m, 2H), 7.19 (t, J=59.2 Hz, 1H), 7.04 (s, 1H), 6.92 (dd, J=2.0, 7.2 Hz, 1H), 6.64 (s, 1H), 4.39-4.16 (m, 1H), 4.12-3.99 (m, 1H), 3.71-3.51 (m, 2H), 3.06-2.87 (m, 1H), 2.83-2.71 (m, 1H), 2.61 (s, 3H), 2.54-2.47 (m, 1H), 2.45 (s, 3H), 2.20-2.05 (m, 2H), 1.01 (t, J=7.2 Hz, 3H)

### Example 480: 5-[2-(difluoromethyl)-4-[[(2R)-1-ethylazetidin-2-yl]methoxy]pyrazol-3-yl]-N-(6-methylpyridazin-3-yl)pyrazolo[1,5-a]pyridin-2-amine

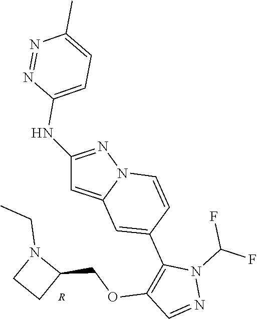

The title compound (13 mg, 3 steps, 3%) was prepared as described in Example 33 Step A followed by a subsequent reductive amination as described in Example 18, except 3-chloro-6-methylpyridazine was used instead of 6-chloro-2,4-dimethylpyridazin-3(2H)-one in Step A. MS (ESI): mass calcd. for C₂₂H₂₄F₂N₈O, 454.2. m/z found, 455.1 [M+H]⁺. ¹H NMR (400 MHz, CDCl₃): 8.61-8.25 (m, 2H), 7.88 (d, J=9.2 Hz, 1H), 7.64 (s, 1H), 7.59 (s, 1H), 7.30-7.27 (m, 1H), 7.18 (t, J=59.2 Hz, 1H), 6.87 (dd, J=2.0, 7.2 Hz, 1H), 6.66 (s, 1H), 4.47-4.23 (m, 1H), 4.13-3.99 (m, 1H), 3.83-3.55 (m, 2H), 3.16-2.98 (m, 1H), 2.91-2.74 (m, 1H), 2.64 (s, 3H), 2.60-2.50 (m, 1H), 2.22-2.13 (m, 2H), 1.04 (t, J=7.2 Hz, 3H) 19F NMR (376 MHz, CDCl3): −90.35-−92.45 (m, 2F)

### Example 481: 5-[2-(difluoromethyl)-4-[[(2R)-1-ethylazetidin-2-yl]methoxy]pyrazol-3-yl]-N-(2-methoxypyrimidin-4-yl)pyrazolo[1,5-a]pyridin-2-amine

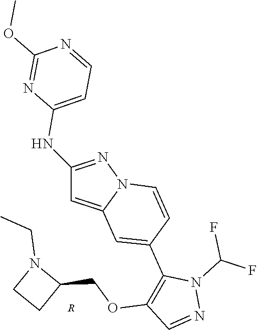

The title compound (23.2 mg, 3 steps, 9%) was prepared as described in Example 33 Step A followed by a subsequent reductive amination as described in Example 18, except 4-chloro-2-methoxypyrimidine was used instead of 6-chloro-2,4-dimethylpyridazin-3(2H)-one in Step A. MS (ESI): mass calcd. for C₂₂H₂₄F₂N₈O₂, 470.2. m/z found, 471.3 [M+H]⁺. ¹H NMR (400 MHz, CDCl₃): 8.36 (d, J=7.2 Hz, 1H), 8.24 (d, J=5.6 Hz, 1H), 7.79 (s, 1H), 7.62 (d, J=0.8 Hz, 1H), 7.59 (s, 1H), 7.19 (t, J=59.2 Hz, 1H), 6.92 (dd, J=2.0, 7.2 Hz, 1H), 6.87 (d, J=4.8 Hz, 1H), 6.77 (s, 1H), 4.09-3.97 (m, 5H), 3.45-3.30 (m, 2H), 2.84-2.71 (m, 1H), 2.70-2.60 (m, 1H), 2.40-2.29 (m, 1H), 2.09-1.92 (m, 2H), 0.93 (t, J=7.2 Hz, 3H) 19F NMR (376 MHz, CDCl3) ¿ −90.32-−92.22 (m, 2F)

### Example 482: 5-[2-(difluoromethyl)-4-[[(2R)-1-ethylazetidin-2-yl]methoxy]pyrazol-3-yl]-N-(6-methylpyrazin-2-yl)pyrazolo[1,5-a]pyridin-2-amine

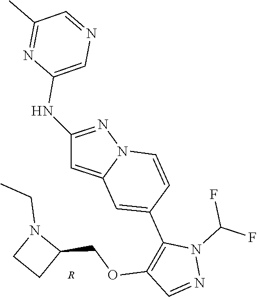

The title compound (48.5 mg, 3 steps, 41%) was prepared as described in Example 33 Step A followed by a subsequent reductive amination as described in Example 18, except 2-chloro-6-methylpyrazine was used instead of 6-chloro-2,4-dimethylpyridazin-3(2H)-one in Step A. MS (ESI): mass calcd. for C₂₂H₂₄F₂N₈O, 454.2. m/z found, 455.4 [M+H]⁺. ¹H NMR (400 MHz, DMSO-d6): 10.24 (s, 1H), 8.66 (d, J=7.2 Hz, 1H), 8.47 (s, 1H), 7.96 (s, 1H), 7.92 (s, 1H), 7.90-7.59 (m, 2H), 6.97 (s, 1H), 6.83 (dd, J=1.6, 7.2 Hz, 1H), 4.17-3.99 (m, 2H), 3.30-3.18 (m, 1H), 2.84-2.56 (m, 2H), 2.42 (s, 3H), 2.34-2.21 (m, 1H), 2.08-1.81 (m, 2H), 0.83 (t, J=6.8 Hz, 3H) 1H NMR (400 MHz, CDCl₃): 8.53 (s, 1H), 8.35 (d, J=7.2 Hz, 1H), 7.99 (s, 1H), 7.62-7.59 (m, 2H), 7.33 (s, 1H), 7.18 (t, J=59.2 Hz, 1H), 6.88 (dd, J=2.0, 7.6 Hz, 1H), 6.75 (s, 1H), 4.25-4.12 (m, 1H), 4.07-3.98 (m, 1H), 3.58-3.42 (m, 2H), 2.95-2.82 (m, 1H), 2.77-2.66 (m, 1H), 2.50 (s, 3H), 2.45-2.36 (m, 1H), 2.12-2.01 (m, 2H), 0.98 (t, J=7.2 Hz, 3H) 19F NMR (376 MHz, DMSO-d6): −91.93-−93.44 (m, 2F)

### Example 483: 5-[2-(difluoromethyl)-4-[[(2R)-1-ethylazetidin-2-yl]methoxy]pyrazol-3-yl]-N-(6-methoxy-5-methyl-pyridazin-3-yl)pyrazolo[1,5-a]pyridin-2-amine

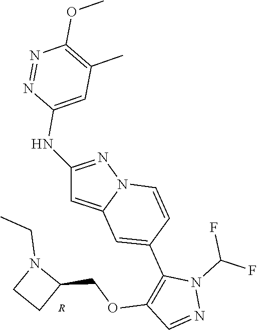

The title compound (31.2 mg, 3 steps, 18%) was prepared as described in Example 33 Step A followed by a subsequent reductive amination as described in Example 18, except 6-chloro-3-methoxy-4-methylpyridazine was used instead of 6-chloro-2,4-dimethylpyridazin-3(2H)-one in Step A. MS (ESI): mass calcd. for C₂₃H₂₆F₂N₈O₂, 484.2. m/z found, 485.2 [M+H]⁺. ¹H NMR (400 MHz, DMSO-d6): 9.81 (s, 1H), 8.61 (d, J=7.2 Hz, 1H), 7.96 (s, 1H), 7.90-7.55 (m, 2H), 7.22 (d, J=1.2 Hz, 1H), 6.91 (s, 1H), 6.83 (dd, J=2.0, 7.2 Hz, 1H), 4.22-3.94 (m, 2H), 3.62 (s, 3H), 3.31-3.12 (m, 2H), 2.85-2.56 (m, 2H), 2.32-2.17 (m, 1H), 2.09 (s, 3H), 2.03-1.81 (m, 2H), 0.90-0.77 (m, 3H) 1H NMR (400 MHz, CD3OD): 8.44 (d, J=7.2 Hz, 1H), 7.80 (s, 1H), 7.64 (d, J=0.8 Hz, 1H), 7.45 (t, J=58.4 Hz, 1H), 7.25 (d, J=1.2 Hz, 1H), 6.92 (d, J=0.4 Hz, 1H), 6.88 (dd, J=1.6, 7.2 Hz, 1H), 4.23-4.08 (m, 2H), 3.81-3.72 (m, 4H), 3.58-3.49 (m, 1H), 3.16-3.03 (m, 1H), 2.86-2.73 (m, 1H), 2.62-2.49 (m, 1H), 2.21-2.15 (m, 4H), 2.14-2.02 (m, 1H), 0.95 (t, J=7.6 Hz, 3H) 19F NMR (376 MHz, DMSO-d6): −92.56-−92.78 (m, 2F)

### Example 484: 6-[[5-[2-(difluoromethyl)-4-[[(2R)-1-ethylazetidin-2-yl]methoxy]pyrazol-3-yl]pyrazolo[1,5-a]pyridin-2-yl]amino]-2,4-dimethyl-pyridazin-3-one

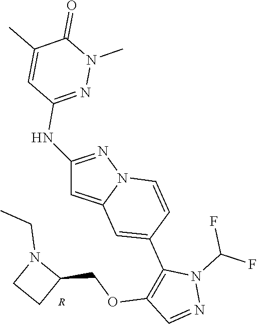

The title compound (35 mg, 3 steps, 8%) was prepared as described in Example 33 Step A followed by a subsequent reductive amination as described in Example 18, except 6-chloro-2,4-dimethylpyridazin-3(2H)-one was used instead of 6-chloro-2,4-dimethylpyridazin-3(2H)-one in Step A. MS (ESI): mass calcd. for C₂₃H₂₆F₂N₈O₂, 484.2. m/z found, 485.2 [M+H]⁺. ¹H NMR (400 MHz, DMSO-d6): 9.81 (s, 1H), 8.61 (d, J=7.2 Hz, 1H), 7.95 (s, 1H), 7.90-7.58 (m, 2H), 7.22 (d, J=1.2 Hz, 1H), 6.92 (s, 1H), 6.82 (dd, J=1.6, 6.8 Hz, 1H), 4.06-4.00 (m, 2H), 3.62 (s, 3H), 3.32-3.19 (m, 2H), 2.72-2.62 (m, 1H), 2.61-2.53 (m, 1H), 2.29-2.17 (m, 1H), 2.09 (d, J=0.8 Hz, 3H), 1.99-1.90 (m, 1H), 1.89-1.78 (m, 1H), 0.80 (t, J=7.2 Hz, 3H) 19F NMR (376 MHz, DMSO-d6): −91.91-−93.39 (m, 2F)

### Example 485: N-(2,6-dimethylpyrimidin-4-yl)-6-[2-methyl-4-[[(2R)-1-methylazetidin-2-yl]methoxy]pyrazol-3-yl]imidazo[1,2-a]pyridin-2-amine

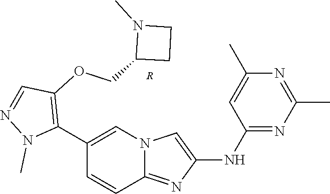

Step A. Tert-butyl (6-bromoimidazo[1,2-a]pyridin-2-yl)carbamate. To a solution of 6-bromoimidazo[1,2-a]pyridin-2-amine (3.00 g, 14.1 mmol, 1 equiv.) in DCM (50 mL) was added TEA (4.30 g, 42.4 mmol, 3 equiv.), DMAP (86.4 mg, 0.707 mmol, 0.05 equiv.) and BoC₂O (3.705 g, 16.98 mmol, 1.2 equiv.) was stirred at 20° C. for 16 hr. The reaction mixture was purified by FCC (PE:EtOAc=3:1) to afford the compound P1 (1.93 g, 32%) as a yellow solid.

Step B. Tert-butyl (6-(4,4,5,5-tetramethyl-1,3,2-dioxaborolan-2-yl)imidazo[1,2-a]pyridin-2-yl)carbamate. A mixture of was tert-butyl (6-bromoimidazo[1,2-a]pyridin-2-yl)carbamate (1.93 g, 6.18 mmol, 1 equiv.) in 1,4-dioxane (40 mL) was added 4,4,4′,4′,5,5,5′,5′-octamethyl-2,2′-bi(1,3,2-dioxaborolane) (1.65 g, 6.49 mmol, 1.05 equiv.), KOAc (1.21 g, 12.4 mmol, 2 equiv.) and Pd(dppf)Cl₂ (452 mg, 0.618 mmol, 0.1 equiv.) under N₂. The reaction was stirred overnight at 90° C. under N2. The reaction mixture was filtered and concentrated to crude product. The crude product was purified by FCC (PE:EtOAc=10:1) to afford the title compound (2.05 g, 92%) as a yellow oil.

Step C. (2-aminoimidazo[1,2-a]pyridin-6-yl)boronic acid. To a solution of tert-butyl (6-(4,4,5,5-tetramethyl-1,3,2-dioxaborolan-2-yl)imidazo[1,2-a]pyridin-2-yl)carbamate (3.2 g, 8.9 mmol, 1 equiv.) in DCM (30 mL) was added TFA (10 mL). The mixture was stirred at 20° C. for hr. The reaction mixture was concentrated to afford pure product.

The product was suspended in water (10 mL), the mixture frozen using dry ice/ethanol, and then lyophilized to dryness to afford the title compound (2.3 g, 89%) as a yellow oil.

Step D. (R)-tert-butyl (R)-2-(((5-(2-aminoimidazo[1,2-a]pyridin-6-yl)-1-methyl-1H-pyrazol-4-yl)oxy)methyl)azetidine-1-carboxylate. A mixture of (R)-tert-butyl 2-(((5-bromo-1-methyl-1H-pyrazol-4-yl)oxy)methyl)azetidine-1-carboxylate (1.70 g, 4.91 mmol, 1 equiv.) (Example 355 Step D) in 1,4-dioxane (40 mL) and H₂O (10 mL) was added 6-(4,4,5,5-tetramethyl-1,3,2-dioxaborolan-2-yl)imidazo[1,2-a]pyridin-2-amine (2.143 g, 5.156 mmol, 1.05 equiv.), K₃PO₄ (1.446 g, 14.73 mmol, 3 equiv.) and Pd(dppf)Cl₂ (360 mg, 0.491 mmol, 0.1 equiv.) under N2. The reaction was stirred 2 hrs at 90° C. under N₂.

The reaction mixture was poured into H₂O (15 mL) and extracted with EA (10 mL×3). The combined organic extracts were washed with 1M NaHCO₃.aq (30 mL), dried over anhydrous Na₂SO₄, filtered, and concentrated to give crude product. The crude product was purified by FCC (MeOH:DCM=1:10) to afford the title compound (610 mg, 31%) as a yellow oil.

Step E. N-(2,6-dimethylpyrimidin-4-yl)-6-[2-methyl-4-[[(2R)-1-methylazetidin-2-yl]methoxy]pyrazol-3-yl]imidazo[1,2-a]pyridin-2-amine. The title compound (23.8 mg, 1000, 3 steps) was prepared as described in Example 22 Steps A-C, except (R)-tert-butyl (R)-2-(((5-(2-aminoimidazo[1,2-a]pyri din-6-yl)-1-methyl-1H-pyrazol-4-yl)oxy)methyl)azetidine-1I-carboxylate was used instead of Intermediate 6. MS (ESI): mass calcd. for C₂₂H₂₆N₈O, 418.2. m/z found, 419.1 [M+H]⁺. ¹H NMR (400 MHz, CDCl₃): 8.29 (s, 1H), 8.18 (s, 1H), 7.63 (br. s., 1H), 7.51 (d, J=9.2 Hz, 1H), 7.36 (s, 1H), 7.25-7.22 (m, 1H), 6.49 (s, 1H), 3.97 (d, J=5.2 Hz, 2H), 3.87 (s, 3H), 3.45-3.36 (m, 1H), 3.35-3.26 (m, 1H), 2.85-2.77 (m, 1H), 2.65 (s, 3H), 2.41 (s, 3H), 2.31 (s, 3H), 2.02-1.95 (m, 2H)

### Additional Compounds

Compounds of Examples 486-507 were prepared and listed in Table 1 below.

The following exemplified compounds were prepared analogous to the method cited in the synthesis method column.

TABLE 1
Additional Compounds

| | | | Synthesis | |
|---|---|---|---|---|
| Example | Structure | NMR | Method | Name |
| 486 | 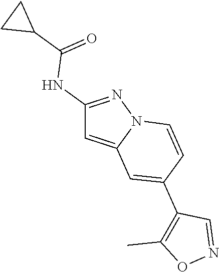 [SMILES: Cc1oncc1-c1ccn2nc(NC(=O)C3CC3)cc2c1] | 1H NMR (400 MHz, DMSO- d6): 11.09 (s, 1H), 8.99 (s, 1H), 8.59 (d, J = 7.2 Hz, 1H), 7.74 (s, 1H), 6.99 (dd, J = 1.6, 7.2 Hz, 1H), 6.85 (s, 1H), 2.65 (s, 3H), 1.97-1.89 (m, 1H), 0.91-0.77 (m, 4H) | Example 28 | N-[5-(5-methylisoxazol-4- yl)pyrazolo[1,5-a]pyridin-2- yl]cyclopropanecarboxamide |
| 487 | 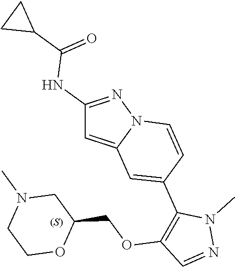 | 1H NMR (400 MHz, DMSO- d6): 11.10 (s, 1H), 8.61 (d, J = 6.8 Hz, 1H), 7.70 (d, J = 1.2 Hz, 1H), 7.46 (s, 1H), 6.96 (dd, J = 1.6, 7.2 Hz, 1H), 6.89 (s, 1H), 3.97-3.88 (m, 2H), 3.85 (s, 3H), 3.78-3.73 (m, 1H), 3.72-3.65 (m, 1H), 3.50-3.42 (m, 1H), 2.72-2.65 (m, 1H), 2.59-2.53 (m, 1H), 2.13 (s, 3H), 1.98-1.89 (m, 2H), 1.83-1.75 (m, 1H), 0.87-0.79 (m, 4H) | Example 8 | N-[5-[2-methyl-4-[(2S)-4- methylmorpholin-2- yl]methoxy]pyrazol-3- yl]pyrazolo[1,5-a]pyridin-2- yl]cyclopropanecarboxamide |
| 488 | 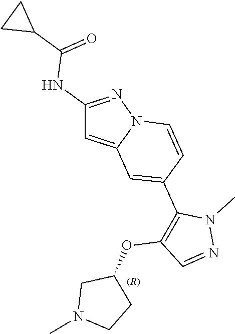 | 1H NMR (400 MHz, DMSO- d6): 11.09 (s, 1H), 8.59 (d, J = 7.6 Hz, 1H), 7.68 (s, 1H), 7.39 (s, 1H), 6.98-6.93 (m, 1H), 6.89 (s, 1H), 4.71-4.62 (m, 1H), 3.84 (s, 3H), 2.71-2.63 (m, 1H), 2.62- 2.56 (m, 2H), 2.36-2.25 (m, 1H), 2.21 (s, 3H), 2.19-2.11 (m, 1H), 1.99-1.88 (m, 1H), 1.84- 1.70 (m, 1H), 0.90-0.77 (m, 4H) | Example 8 | N-[5-[2-methyl-4-[(3R)-1- methylpyrrolidin-3-yl]oxy- pyrazol-3-yl]pyrazolo[1,5- a]pyridin-2- yl]cyclopropanecarboxamide |
| 489 | 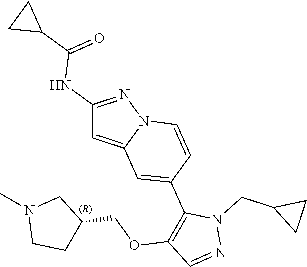 | 1H NMR (400 MHz, CDCl3): 8.60 (br. s., 1H), 8.31 (d, J = 7.2 Hz, 1H), 7.48 (s, 1H), 7.36 (s, 1H), 7.02 (s, 1H), 6.81 (dd, J = 0.8, 7.2 Hz, 1H), 4.02 (d, J = 6.8 Hz, 2H), 3.86 (d, J = 6.8 Hz, 2H), 2.67-2.45 (m, 4H), 2.43- 2.37 (m, 1H), 2.33 (s, 3H), 2.05- 1.92 (m, 1H), 1.65-1.45 (m, 2H), 1.21-1.08 (m, 3H), 0.97- 0.87 (m, 2H), 0.54-0.45 (m, 2H), 0.27-0.17 (m, 2H) | Example 260 | N-[5-[2-(cyclopropylmethyl)-4- [[(3R)-1-methylpyrrolidin-3- yl]methoxy]pyrazol-3- yl]pyrazolo[1,5-a]pyridin-2- yl]cyclopropanecarboxamide |
| 490 | 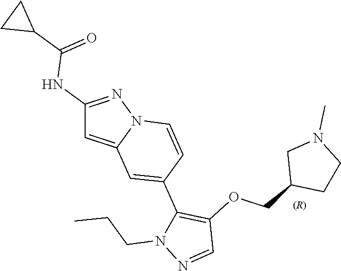 | 1H NMR (400 MHz, CD3OD): 8.46 (d, J = 7.2 Hz, 1H), 7.61- 7.53 (m, 1H), 7.45 (s, 1H), 6.92 (s, 1H), 6.87 (dd, J = 1.6, 7.2 Hz, 1H), 4.13 (t, J = 7.2 Hz, 2H), 3.92-3.83 (m, 2H), 2.72-2.65 (m, 1H), 2.64-2.45 (m, 3H), 2.42-2.33 (m, 1H), 2.30 (s, 3H), 2.03-1.94 (m, 1H), 1.92-1.83 (m, 1H), 1.79-1.66 (m, 2H), 1.61-1.51 (m, 1H), 1.05-0.97 (m, 2H), 0.94-0.86 (m, 2H), 0.77 (t, J = 7.2 Hz, 3H) | Example 260 | N-[5-[4-[[(3R)-1- methylpyrrolidin-3-yl]methoxy]- 2-propyl-pyrazol-3- yl]pyrazolo[1,5-a]pyridin-2- yl]cyclopropanecarboxamide |
| 491 | 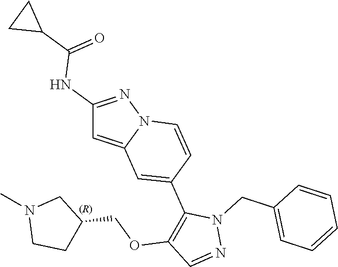 | 1H NMR (400 MHz, CD3OD): 8.39 (d, J = 7.2 Hz, 1H), 7.56 (s, 1H), 7.45 (d, J = 0.4 Hz, 1H), 7.29-7.16 (m, 3H), 7.04-6.94 (m, 2H), 6.83 (s, 1H), 6.77 (dd, J = 1.6, 6.8 Hz, 1H), 5.40 (s, 2H), 4.08-3.90 (m, 2H), 3.17-3.07 (m, 1H), 3.04-2.88 (m, 2H), 2.87-2.69 (m, 2H), 2.60 (s, 3H), 2.19-2.05 (m, 1H), 1.90-1.83 (m, 1H), 1.82-1.70 (m, 1H), 1.02-0.95 (m, 2H), 0.94-0.86 (m, 2H) | Example 260 | N-[5-[2-benzyl-4-[[(3R)-1- methylpyrrolidin-3- yl]methoxy]pyrazol-3- yl]pyrazolo[1,5-a]pyridin-2- yl]cyclopropanecarboxamide |
| 492 | 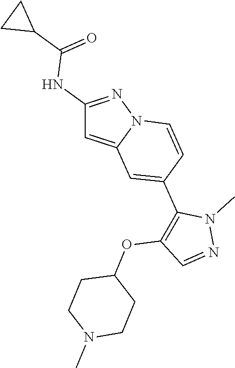 [SMILES: CN1CCC(Oc2cnn(C)c2-c2ccn3nc(NC(=O)C4CC4)cc3c2)CC1] | 1H NMR (500 MHz, DMSO-d6) d 11.00 (s, 1H), 8.53 (dt, J = 7.2, 1.0 Hz, 1H), 7.62 (dd, J = 2.0, 0.9 Hz, 1H), 7.36 (s, 1H), 6.89 (dd, J = 7.2, 2.0 Hz, 1H), 6.84- 6.75 (m, 1H), 3.93 (dq, J = 7.5, 3.8 Hz, 1H), 3.77 (s, 3H), 2.38 (s, 2H), 2.02 (s, 5H), 1.86 (dp, J = 7.8, 4.6 Hz, 1H), 1.80-1.68 (m, 2H), 1.51 (dtd, J = 12.0, 8.1, 3.5 Hz, 2H), 1.11 (d, J = 6.3 Hz, 2H), 0.80-0.67 (m, 4H). | Example 8 | N-[5-[2-methyl-4-[(1-methyl-4- piperidyl)oxy]pyrazol-3- yl]pyrazolo[1,5-a]pyridin-2- yl]cyclopropanecarboxamide |
| 493 | 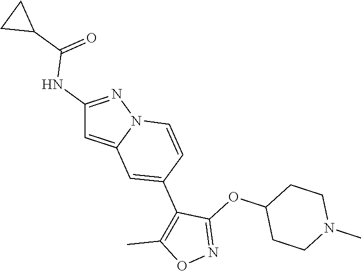 [SMILES: Cc1onc(OC2CCN(C)CC2)c1-c1ccn2nc(NC(=O)C3CC3)cc2c1] | 1H NMR (500 MHz, DMSO-d6) d 11.05 (s, 1H), 8.58 (d, J = 7.2 Hz, 1H), 7.64 (dd, J = 2.0, 0.9 Hz, 1H), 6.91 (dd, J = 7.2, 2.0 Hz, 1H), 6.85 (s, 1H), 4.71 (dd, J = 8.1, 4.5 Hz, 1H), 2.51 (s, 3H), 2.27-2.19 (m, 2H), 2.17 (s, 3H), 2.06-1.97 (m, 2H), 1.95- 1.89 (m, 1H), 1.84-1.73 (m, 2H), 0.87-0.76 (m, 4H). | Example 28 | N-[5-[5-methyl-3-[(1-methyl-4- piperidyl)oxy]isoxazol-4- yl]pyrazolo[1,5-a]pyridin-2- yl]cyclopropanecarboxamide |
| 494 | 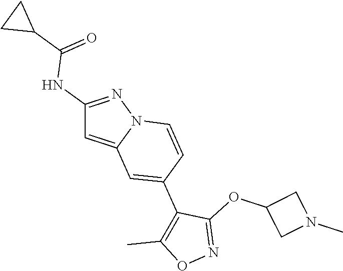 [SMILES: Cc1onc(OC2CN(C)C2)c1-c1ccn2nc(NC(=O)C3CC3)cc2c1] | 1H NMR (500 MHz, DMSO-d6) d 11.04 (s, 1H), 8.61-8.55 (m, 1H), 7.65 (dd, J = 2.0, 1.0 Hz, 1H), 6.90 (dd, J = 7.2, 2.0 Hz, 1H), 6.87 (s, 1H), 5.06-4.94 (m, 1H), 3.70-3.60 (m, 2H), 3.14-3.04 (m, 2H), 2.51 (s, 3H), 2.28 (s, 3H), 1.98-1.85 (m, 1H), 0.87-0.75 (m, 4H). | Example 28 | N-[5-[5-methyl-3-(1- methylazetidin-3-yl)oxy- isoxazol-4-yl]pyrazolo[1,5- a]pyridin-2- yl]cyclopropanecarboxamide |
| 495 | 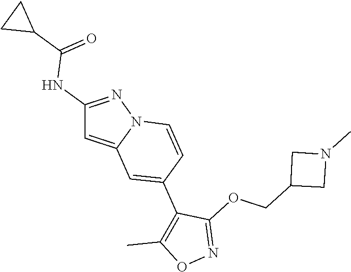 [SMILES: Cc1onc(OCC2CN(C)C2)c1-c1ccn2nc(NC(=O)C3CC3)cc2c1] | 1H NMR (500 MHz, DMSO-d6) δ 11.06 (s, 1H), 8.60 (dd, J = 7.1, 0.9 Hz, 1H), 7.68 (dd, J = 2.0, 0.9 Hz, 1H), 6.91 (dd, J = 7.2, 2.0 Hz, 1H), 6.86 (s, 1H), 4.48 (s, 2H), 4.07 (p, J = 5.8 Hz, 1H), 3.39 (td, J = 6.0, 2.0 Hz, 2H), 2.69 (td, J = 5.8, 2.0 Hz, 2H), 2.51 (s, 3H), 2.16 (s, 3H), 1.96- 1.88 (m, 1H), 0.88-0.77 (m, 4H). | Example 28 | N-[5-[5-methyl-3-[(1- methylazetidin-3- yl)oxymethyl]isoxazol-4- yl]pyrazolo[1,5-a]pyridin-2- yl]cyclopropanecarboxamide |
| 496 | 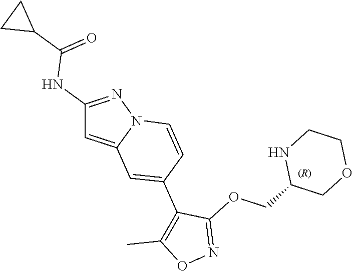 | 1H NMR (500 MHz, DMSO-d6) d 11.05 (s, 1H), 8.57 (dd, J = 7.2, 1.0 Hz, 1H), 7.65 (dd, J = 2.0, 1.0 Hz, 1H), 6.91 (dd, J = 7.2, 2.0 Hz, 1H), 6.86 (s, 1H), 4.22- 4.06 (m, 2H), 3.79 (dd, J = 10.8, 3.1 Hz, 1H), 3.69-3.61 (m, 1H), 3.40-3.34 (m, 1H), 3.24 (dd, J = 10.8, 9.2 Hz, 1H), 3.17- 3.05 (m, 1H), 2.83-2.69 (m, 2H), 2.52 (s, 3H), 1.96-1.86 (m, 1H), 0.90-0.77 (m, 4H). | Example 28 | N-[5-[5-methyl-3-[[(3R)- morpholin-3- yl]methoxy]isoxazol-4- yl]pyrazolo[1,5-a]pyridin-2- yl]cyclopropanecarboxamide |
| 497 | 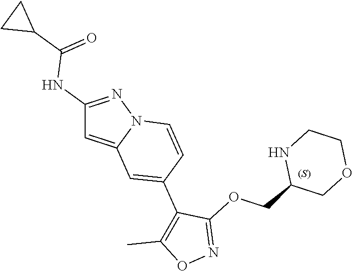 | 1H NMR (500 MHz, DMSO-d6) d 11.05 (s, 1H), 8.57 (dd, J = 7.3, 1.0 Hz, 1H), 7.65 (dd, J = 2.0, 0.9 Hz, 1H), 6.91 (dd, J = 7.2, 2.0 Hz, 1H), 6.86 (s, 1H), 4.22- 4.08 (m, 2H), 3.79 (dd, J = 10.8, 3.1 Hz, 1H), 3.65 (dt, J = 10.9, 2.9 Hz, 1H), 3.40-3.34 (m, 1H), 3.24 (dd, J = 10.8, 9.2 Hz, 1H), 3.14-3.06 (m, 1H), 2.83- 2.68 (m, 2H), 2.52 (s, 3H), 1.97- 1.84 (m, 1H), 0.87-0.77 (m, 4H). | Example 28 | N-[5-[5-methyl-3-[(3S)- morpholin-3- yl]methoxy]isoxazol-4- yl]pyrazolo[1,5-a]pyridin-2- yl]cyclopropanecarboxamide |
| 498 | 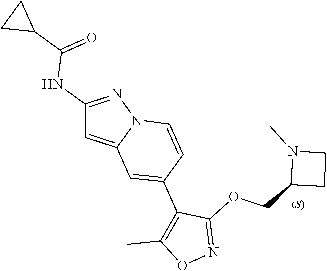 | 1H NMR (400 MHz, DMSO-d6) d 11.05 (s, 1H), 8.62-8.48 (m, 1H), 7.65 (dd, J = 2.0, 1.0 Hz, 1H), 6.91 (dd, J = 7.2, 2.0 Hz, 1H), 6.85-6.79 (m, 1H), 4.27 (dd, J = 10.7, 3.9 Hz, 1H), 4.20 (dd, J = 10.6, 6.5 Hz, 1H), 3.38- 3.33 (m, 1H), 3.29-3.24 (m, 1H), 2.79-2.68 (m, 1H), 2.52 (s, 3H), 2.23 (s, 3H), 2.03-1.87 (m, 3H), 0.87-0.75 (m, 4H). | Example 28 | N-[5-[5-methyl-3-[(2S)-1- methylazetidin-2- yl]methoxy]isoxazol-4- yl]pyrazolo[1,5-a]pyridin-2- yl]cyclopropanecarboxamide |
| 499 | 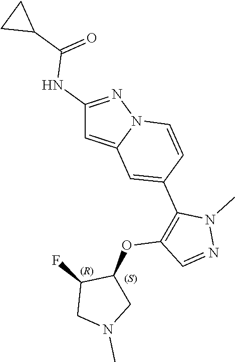 | 1H NMR (400 MHz, DMSO- d6): 11.11 (s, 1H), 8.61 (d, J = 7.2 Hz, 1H), 7.70 (s, 1H), 7.53 (s, 1H), 6.96 (dd, J = 2.0, 7.2 Hz, 1H), 6.89 (s, 1H), 5.33-5.12 (m, 1H), 4.71-4.57 (m, 1H), 3.85 (s, 3H), 2.98-2.83 (m, 2H), 2.77- 2.62 (m, 2H), 2.26 (s, 3H), 1.99- 1.87 (m, 1H), 0.90-0.76 (m, 4H) 19F NMR (376 MHz, DMSO-d6): −194.68-−195.31 (m, 1F) | Example 8 | N-[5-[4-[(3S,4R)-4-fluoro-1- methyl-pyrrolidin-3-ylJoxy-2- methyl-pyrazol-3- yl]pyrazolo[1,5-a]pyridin-2- yl]cyclopropanecarboxamide |
| 500 | 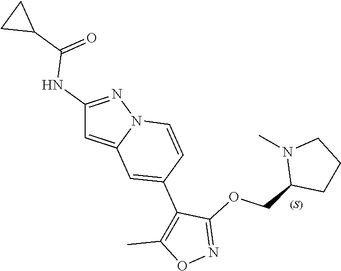 | 1H NMR (400 MHz, CD3OD): 8.40 (d, J = 7.2 Hz, 1H), 7.62 (s, 1H), 6.94 (dd, J = 1.6, 7.2 Hz, 1H), 6.84 (s, 1H), 4.37-4.25 (m, 2H), 3.10-3.03 (m, 1H), 2.86- 2.77 (m, 1H), 2.52 (s, 3H), 2.44 (s, 3H), 2.39-2.29 (m, 1H), 2.15- 2.02 (m, 1H), 1.90-1.66 (m, 4H), 1.02-0.95 (m, 2H), 0.94- 0.87 (m, 2H) | Example 28 | N-[5-[5-methyl-3-[[(2S)-1- methylpyrrolidin-2- yl]methoxy]isoxazol-4- yl]pyrazolo[1,5-a]pyridin-2- yl]cyclopropanecarboxamide |
| 501 | 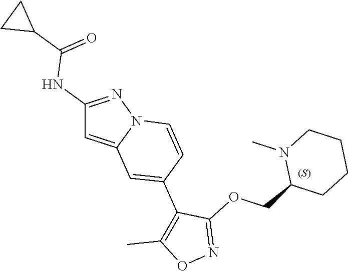 | 1H NMR (400 MHz, DMSO- d6): 11.10 (s, 1H), 8.59 (d, J = 7.2 Hz, 1H), 7.67 (d, J = 1.2 Hz, 1H), 6.92 (dd, J = 2.0, 7.2 Hz, 1H), 6.84 (s, 1H), 4.36-4.21 (m, 2H), 2.82-2.71 (m, 1H), 2.53 (s, 3H), 2.29-2.18 (m, 4H), 2.11- 1.97 (m, 1H), 1.97-1.87 (m, 1H), 1.77-1.63 (m, 2H), 1.60- 1.50 (m, 1H), 1.49-1.31 (m, 2H), 1.29-1.17 (m, 1H), 0.92- 0.76 (m, 4H) | Example 28 | N-[5-[5-methyl-3-[[(2S)-1- methyl-2- piperidyl]methoxy]isoxazol-4- yl]pyrazolo[1,5-a]pyridin-2- yl]cyclopropanecarboxamide |
| 502 | 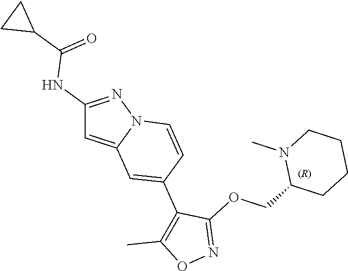 | 1H NMR (400 MHz, DMSO- d6): 11.09 (s, 1H), 8.59 (d, J = 7.6 Hz, 1H), 7.67 (d, J = 0.8 Hz, 1H), 6.92 (dd, J = 1.6, 6.8 Hz, 1H), 6.84 (s, 1H), 4.34-4.24 (m, 2H), 2.79-2.72 (m, 1H), 2.54- 2.52 (m, 3H), 2.28-2.20 (m, 4H), 2.08-1.98 (m, 1H), 1.97- 1.89 (m, 1H), 1.74-1.65 (m, 2H), 1.58-1.50 (m, 1H), 1.49- 1.32 (m, 2H), 1.30-1.19 (m, 1H), 0.87-0.78 (m, 4H) | Example 28 | N-[5-[5-methyl-3-[[(2R)-1- methyl-2- piperidyl]methoxy]isoxazol-4- yl]pyrazolo[1,5-a]pyridin-2- yl]cyclopropanecarboxamide |
| 503 | 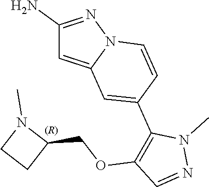 | 1H NMR (400 MHz, DMSO- d6): 8.39-8.29 (m, 1H), 7.49- 7.38 (m, 2H), 6.74-6.63 (m, 1H), 5.72 (s, 1H), 5.39 (s, 2H), 4.05-3.98 (m, 2H), 3.83 (s, 3H), 3.62-3.53 (m, 2H), 3.07-2.93 (m, 1H), 2.32 (s, 3H), 2.07-1.93 (m, 2H) | Example 8 | 5-[2-methyl-4-[[(2R)-1- methylazetidin-2- yl]methoxy]pyrazol-3- yl]pyrazolo[1,5-a]pyridin-2- amine |
| 504 | 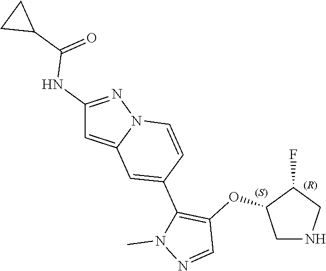 | 1H NMR (400 MHz, DMSO- d6): 11.15 (s, 1H), 9.99-9.72 (m, 2H), 8.63 (d, J = 7.2 Hz, 1H), 7.73 (d, J = 0.8 Hz, 1H), 7.62 (s, 1H), 6.96 (dd, J = 2.0, 7.2 Hz, 1H), 6.92 (s, 1H), 5.53- 5.34 (m, 1H), 4.97-4.81 (m, 1H), 3.86 (s, 3H), 3.58-3.49 (m, 2H), 3.49-3.38 (m, 1H), 3.26- 3.13 (m, 1H), 2.01-1.87 (m, 1H), 0.92-0.75 (m, 4H) | Example 4 | N-[5-[4-[(3S,4R)-4- fluoropyrrolidin-3-yl]oxy-2- methyl-pyrazol-3- yl]pyrazolo[1,5-a]pyridin-2- yl]cyclopropanecarboxamide |
| 505 | 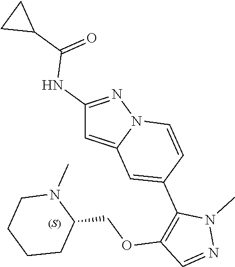 | 1H NMR (400MHz, DMSO-d6): 11.12 (s, 1H), 10.92-10.46 (m, 1H), 8.60 (d, J = 6.8 Hz, 1H), 7.79-7.74 (m, 1H), 7.60-7.55 (m, 1H), 7.03-6.96 (m, 1H), 6.92 (s, 1H), 4.36-4.09 (m, 2H), 3.90-3.63 (m, 3H), 3.45-3.15 (m, 2H), 3.07-2.94 (m, 1H), 2.81-2.68 (m, 3H), 2.02-1.58 (m, 6H), 1.54-1.37 (m, 1H), 0.90-0.77 (m, 4H) | Example 8 | N-[5-[2-methyl-4-[(2S)-1- methyl-2- piperidyl[methoxy]pyrazol-3- yl]pyrazolo[1,5-a]pyridin-2- yl]cyclopropanecarboxamide |
| 506 | 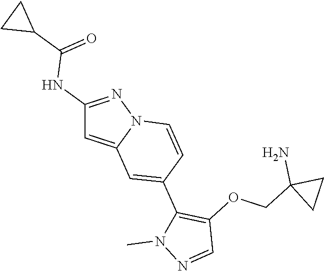 [SMILES: Cn1ncc(OCC2(N)CC2)c1-c1ccn2nc(NC(=O)C3CC3)cc2c1] | 1H NMR (400 MHz, DMSO- d6): 11.10 (s, 1H), 8.60 (d, J = 7.2 Hz, 1H), 7.73 (s, 1H), 7.41 (s, 1H), 7.00 (dd, J = 1.6, 7.2 Hz, 1H), 6.90 (s, 1H), 3.85 (s, 3H), 3.81 (s, 2H), 2.16-1.86 (m, 3H), 0.88-0.77 (m, 4H), 0.52-0.43 (m, 4H) | Example 4 | N-[5-[4-[(1- aminocyclopropyl)methoxy]-2- methyl-pyrazol-3- yl]pyrazolo[1,5-a]pyridin-2- yl]cyclopropanecarboxamide |
| 507 | 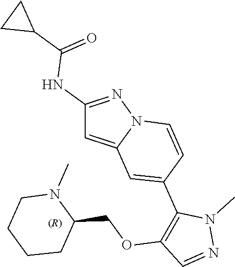 | 1H NMR (400 MHz, CD3OD): 8.46 (d, J = 7.2 Hz, 1H), 7.64 (s, 1H), 7.45 (s, 1H), 6.95 (dd, J = 2.0, 7.2 Hz, 1H), 6.92 (s, 1H), 4.05-3.97 (m, 2H), 3.87 (s, 3H), 2.91-2.83 (m, 1H), 2.33 (s, 3H), 2.27-2.15 (m, 1H), 1.94-1.83 (m, 1H), 1.80-1.69 (m, 2H), 1.69-1.40 (m, 3H), 1.39-1.21 (m, 2H), 1.05-0.96 (m, 2H), 0.95-0.86 (m, 2H) | Example 8 | N-[5-[2-methyl-4-[[(2R)-1- methyl-2- piperidyl]methoxy]pyrazol-3- yl]pyrazolo[1,5-a]pyridin-2- yl]cyclopropanecarboxamide |

### Biological Testing Examples

### Test Example 1: SIK2 Enzymatic Assay Version 1

All steps were performed at room temperature. Compounds were pre-plated onto an assay plate using an Echo acoustic dispenser (Labcyte, E550 or E555) at 20 nL per well. Enzyme prepared in Assay Buffer at 0.6 nM (2× final concentration) was added at 2 μL per well using the Multidrop Combi (Thermo Fisher-used for all additions in this assay) to the assay plate, spun for 1 min at 800-850 rpm (Allegra 6KR Beckman Coulter centrifuge), and incubated with compound for 30 minutes. Assay Buffer containing 160 μM ATP and 140 μM HDAC5tide (2× of final concentration for each substrate) was added at 2 μL per well to the assay plate and then spun as above and incubated for 4 hours.

One column of wells was dedicated to a no-peptide negative control (containing only ATP). ADPGlo Reagent (prepared and aliquoted as per manufacturer instructions) was then added at 2 μL per well, spun and incubated for 40 min. ADPGlo Detection (prepared and aliquoted as per manufacturer instructions) was added at 2 μL per well to the assay plate, which was spun and incubated for at least 30 min. The plate was then read on the Pherastar (BMG Labtech) using a luminescence read. Gain was set by the 0.1 mM concentration of ADP wells in the ADP/ATP standard curve plate generated as per manufacturer instructions.

The results are shown in Table 2 below.

### Test Example 1: SIK2 Enzymatic Assay High Version 2 (High ATP)

All steps were performed at room temperature. Compounds were pre-plated onto an assay plate using an Echo acoustic dispenser (Labcyte, E550 or E555) at 80 nL per well. Enzyme prepared in assay buffer at 6 nM (2× final concentration) was added at 4 μL per well using the Multidrop Combi (Thermo Fisher-used for all additions in this assay) to the assay plate, spun for 1 min at 1000 rpm (Allegra 6KR Beckman Coulter centrifuge), and incubated with compound for 30 minutes. Assay buffer containing 6 mM ATP and 400 μM HDAC5tide (2× of final concentration for each substrate) was added at 4 μL per well to the assay plate and then spun as above and incubated for 2 hours. Note that typically one column of wells was dedicated to a no-enzyme negative control (containing ATP and peptide) and another dedicated to DMSO only (maximal enzyme activity). ADP-Glo Max Reagent (prepared and aliquoted as per manufacturer instructions) was then added at 8 μL per well, spun and incubated for 2 hours. ADP-Glo Max Detection (prepared and aliquoted as per manufacturer instructions) was added at 8 μL per well to the assay plate, which was spun and incubated for 1 hour. The plate was then read on the Pherastar (BMG Labtech) using a luminescence read. Gain was set between 2200-2500.

The results are shown in Table 2 below.

TABLE 2
SIK2 Enzymatic Assay Data

| Compound | | |
|---|---|---|
| Example | SIK2 ADP Glo IC₅₀ (μM) | SIK2 High ATP (μM) |
| 1 | | <0.00083837 |
| 1 | | 0.2078 |
| 2 | | <0.00084675 |
| 3 | | 0.0085 |
| 4 | | 0.0076 |
| 5 | | 0.0109 |
| 6 | | 0.0140 |
| 7 | | 0.0787 |
| 8 | | 0.0189 |
| 9 | | 0.0359 |
| 10 | | 0.9882 |
| 11 | | 0.1171 |
| 12 | | 0.1169 |
| 13 | | 0.0715 |
| 14 | | 0.0017 |
| 15 | | 0.0091 |
| 16 | | 0.2192 |
| 17 | | <0.00084675 |
| 18 | | 0.0010 |
| 19 | | 0.0012 |
| 20 | | <0.00084675 |
| 21 | | 0.0009 |
| 22 | | <0.00084675 |
| 23 | | 0.0012 |
| 24 | | 0.0349 |
| 25 | | 0.0058 |
| 26 | | 0.3194 |
| 27 | | 0.0118 |
| 28 | | 0.0396 |
| 29 | | 0.3595 |
| 30 | | 0.0052 |
| 31 | | 0.0010 |
| 32 | | 0.0066 |
| 33 | | 0.0013 |
| 34 | | 0.0092 |
| 35 | | 0.0192 |
| 36 | | 0.0103 |
| 37 | | 0.0429 |
| 38 | | 0.0022 |
| 38 | | 0.0033 |
| 39 | | 0.0034 |
| 40 | | 0.4439 |
| 41 | | 0.0146 |
| 42 | | 0.0146 |
| 43 | | 0.0173 |
| 44 | | 0.0176 |
| 45 | | 0.0241 |
| 46 | | 0.0025 |
| 47 | | 0.0877 |
| 48 | | 0.2693 |
| 49 | | 0.0053 |
| 50 | | 0.0026 |
| 51 | | 0.0470 |
| 52 | | 0.0036 |
| 53 | | 0.0683 |
| 54 | | 0.1776 |
| 55 | | 0.0013 |
| 56 | | 2.5048 |
| 57 | | 0.4713 |
| 58 | | 0.0578 |
| 59 | | 0.6897 |
| 60 | | 0.2300 |
| 61 | | 1.1446 |
| 62 | | 0.0009 |
| 63 | | 0.0011 |
| 64 | | 0.0020 |
| 65 | | <0.00084675 |
| 66 | | 0.0014 |
| 67 | | 0.0521 |
| 68 | | <0.00084675 |
| 69 | | 0.0303 |
| 70 | | 0.2188 |
| 71 | | 0.0223 |
| 72 | | 0.0521 |
| 73 | | <0.00084675 |
| 74 | | 0.1439 |
| 75 | | 0.0350 |
| 76 | | 0.0122 |
| 77 | | 0.0129 |
| 78 | | 0.0200 |
| 79 | | 0.1195 |
| 80 | | 0.1461 |
| 81 | | 0.0097 |
| 82 | | 3.9506 |
| 83 | | 0.0109 |
| 84 | | 0.2042 |
| 85 | | 0.0321 |
| 86 | | 0.0013 |
| 87 | | 0.0165 |
| 88 | | 0.0115 |
| 89 | | 0.0159 |
| 90 | | 0.0144 |
| 91 | | 0.0064 |
| 92 | | 0.0043 |
| 93 | | 0.0047 |
| 94 | | 0.0103 |
| 95 | | 0.0053 |
| 96 | | 0.0068 |
| 97 | | 0.0029 |
| 98 | | 0.0079 |
| 99 | | 0.0109 |
| 100 | | 0.0124 |
| 101 | | 0.0072 |
| 102 | | <0.00084675 |
| 103 | | <0.00084675 |
| 104 | | 0.0169 |
| 105 | | 0.0431 |
| 106 | | 0.0024 |
| 107 | | 0.0033 |
| 108 | | 0.0033 |
| 109 | | 0.0062 |
| 110 | | 0.0028 |
| 111 | | 0.0013 |
| 112 | | 0.0027 |
| 113 | | 0.0223 |
| 114 | | 0.0011 |
| 115 | | 0.0090 |
| 116 | | <0.00084675 |
| 117 | | 0.0088 |
| 118 | | 0.0019 |
| 119 | | 0.0016 |
| 120 | | 0.0237 |
| 121 | | 0.0010 |
| 122 | | 0.0036 |
| 123 | | 0.0084 |
| 124 | | 0.0149 |
| 125 | | 0.0013 |
| 126 | | 0.0041 |
| 127 | | 0.0110 |
| 128 | | <0.00084675 |
| 129 | | <0.00084675 |
| 130 | | <0.00084675 |
| 131 | | <0.00084675 |
| 132 | | 0.0089 |
| 133 | | 0.0071 |
| 134 | | 0.0028 |
| 135 | | 0.0058 |
| 136 | | 0.0023 |
| 137 | | 0.0169 |
| 138 | | 0.0403 |
| 139 | | 0.0197 |
| 140 | | 0.0012 |
| 141 | | 0.0010 |
| 142 | | 0.0028 |
| 143 | | 0.0024 |
| 144 | | <0.00084675 |
| 145 | | <0.00084675 |
| 146 | | <0.00084675 |
| 147 | | 0.0009 |
| 148 | | <0.00084675 |
| 149 | | 0.0020 |
| 150 | | 0.0076 |
| 151 | | <0.00084675 |
| 152 | | <0.00084675 |
| 153 | | <0.00084675 |
| 154 | | 0.0079 |
| 155 | | 0.0066 |
| 156 | | 0.0031 |
| 157 | | 0.0111 |
| 158 | | 0.0477 |
| 159 | | 0.0242 |
| 160 | | 0.0025 |
| 160 | | 0.0036 |
| 161 | | <0.00084675 |
| 162 | | <0.00084675 |
| 163 | | 0.0022 |
| 164 | | 0.0015 |
| 165 | | 0.0262 |
| 166 | | 0.0018 |
| 167 | | 0.0027 |
| 168 | | 0.0230 |
| 169 | | 0.0387 |
| 170 | | 0.0009 |
| 171 | | <0.00084675 |
| 172 | | <0.00084675 |
| 173 | | <0.00084675 |
| 174 | | <0.00084675 |
| 175 | | <0.00084675 |
| 176 | | 0.0727 |
| 177 | | 0.0015 |
| 178 | | <0.00084675 |
| 179 | | <0.00084675 |
| 180 | | 0.0021 |
| 181 | | 0.0022 |
| 182 | | 0.0344 |
| 183 | | 0.0117 |
| 184 | | 0.0145 |
| 185 | | 0.0183 |
| 186 | | 0.0073 |
| 187 | | 0.0235 |
| 188 | | 0.0077 |
| 189 | | 0.0120 |
| 190 | | 0.0265 |
| 191 | | 0.0113 |
| 192 | | 0.0196 |
| 193 | | 0.0212 |
| 194 | | 0.0047 |
| 195 | | 0.0035 |
| 196 | | 0.0247 |
| 197 | | 0.0281 |
| 198 | | 0.0287 |
| 199 | | 0.0141 |
| 200 | | 0.0294 |
| 201 | | 0.0421 |
| 202 | | 0.0123 |
| 203 | | 0.0274 |
| 204 | | 0.0174 |
| 205 | | 0.0235 |
| 206 | | 0.0350 |
| 207 | | 0.0076 |
| 208 | | 0.0115 |
| 209 | | 0.0149 |
| 210 | | 0.0075 |
| 211 | | 0.0325 |
| 212 | | 0.0464 |
| 213 | | 0.0153 |
| 214 | | 0.0158 |
| 215 | | 0.0275 |
| 216 | | 0.0296 |
| 217 | | 0.0241 |
| 218 | | 0.0495 |
| 219 | | 0.0443 |
| 220 | | 0.0049 |
| 221 | | 0.0232 |
| 222 | | 0.0275 |
| 223 | | 0.0307 |
| 224 | | 0.0214 |
| 225 | | 0.0120 |
| 226 | | 0.0128 |
| 227 | | 0.0349 |
| 228 | | 0.0164 |
| 229 | | 0.0238 |
| 230 | | 0.0418 |
| 231 | | 0.0469 |
| 232 | | 0.0332 |
| 233 | | 0.0302 |
| 234 | | 0.0011 |
| 235 | | <0.00084675 |
| 236 | | 0.0291 |
| 237 | | 0.0107 |
| 238 | | 0.0348 |
| 239 | | 0.0227 |
| 240 | | 0.0287 |
| 241 | | 0.0035 |
| 242 | | 0.0083 |
| 243 | | 0.0113 |
| 244 | | 0.0063 |
| 245 | | 0.0137 |
| 246 | | 0.0016 |
| 247 | | 0.0027 |
| 248 | | 0.0518 |
| 249 | | 0.0255 |
| 250 | | 0.0015 |
| 251 | | 0.0456 |
| 252 | | 0.0094 |
| 253 | | 0.0024 |
| 254 | | 0.0015 |
| 255 | | 0.0518 |
| 256 | | 0.0420 |
| 257 | | 0.0235 |
| 258 | | 0.0073 |
| 259 | | 0.0465 |
| 260 | | 0.0376 |
| 261 | | 0.0055 |
| 262 | | 0.0047 |
| 263 | | 0.0402 |
| 264 | | 0.0077 |
| 265 | | 0.0305 |
| 266 | | 0.0395 |
| 267 | | 0.0298 |
| 268 | | 0.0365 |
| 269 | | 0.0332 |
| 270 | | 0.0477 |
| 271 | | 0.0408 |
| 272 | | 0.0435 |
| 273 | | 0.0181 |
| 274 | | 0.0009 |
| 275 | | 0.0322 |
| 276 | | 0.0009 |
| 277 | | 0.0014 |
| 278 | | 0.0010 |
| 279 | | 0.0018 |
| 280 | | 0.0358 |
| 281 | | 0.0291 |
| 282 | | 0.0113 |
| 283 | | 0.0034 |
| 284 | | 0.0165 |
| 285 | | 0.0185 |
| 286 | | 0.0235 |
| 287 | | 0.0372 |
| 288 | | 0.0168 |
| 289 | | 0.0014 |
| 290 | | <0.00084675 |
| 291 | | 0.0312 |
| 292 | | <0.00084675 |
| 293 | | <0.00084675 |
| 294 | | 0.0026 |
| 295 | | 0.0245 |
| 296 | | 0.0062 |
| 297 | | <0.00084675 |
| 298 | | 0.0392 |
| 299 | | 0.0311 |
| 300 | | 0.0108 |
| 301 | | 0.0152 |
| 302 | | 0.0283 |
| 303 | | 0.0023 |
| 304 | | 0.0457 |
| 305 | | 0.0161 |
| 306 | | 0.0361 |
| 307 | | 0.0309 |
| 308 | | 0.0153 |
| 309 | | 0.0029 |
| 310 | | 0.0040 |
| 311 | | 0.0014 |
| 312 | | 0.0443 |
| 313 | | 0.0296 |
| 314 | | 0.0304 |
| 315 | | 0.0141 |
| 316 | | 0.0503 |
| 317 | | 0.0678 |
| 318 | | 0.0135 |
| 319 | | 0.0518 |
| 320 | | 0.0508 |
| 321 | | 0.0526 |
| 322 | | 0.0463 |
| 323 | | 0.0256 |
| 324 | | 0.0238 |
| 325 | | 0.0632 |
| 326 | | 0.0600 |
| 327 | | 0.0428 |
| 328 | | 0.0618 |
| 329 | | 0.0350 |
| 330 | | 0.0352 |
| 331 | | 0.0324 |
| 332 | | 0.0583 |
| 333 | | 0.0540 |
| 334 | | 0.0551 |
| 335 | | 0.0687 |
| 336 | | 0.0664 |
| 337 | | 0.0544 |
| 338 | | 0.0530 |
| 339 | | 0.0685 |
| 340 | | 0.0554 |
| 341 | | 0.0577 |
| 342 | | 0.0688 |
| 343 | | 0.0548 |
| 344 | | 0.0560 |
| 345 | | 0.0582 |
| 346 | | 0.0589 |
| 347 | | 0.0680 |
| 348 | | 0.0553 |
| 349 | | 0.0559 |
| 350 | | 0.0662 |
| 351 | | 0.0526 |
| 352 | | 0.0668 |
| 353 | | 0.0679 |
| 354 | | 0.0673 |
| 355 | | <0.00084675 |
| 356 | | 0.0591 |
| 357 | | 0.0619 |
| 358 | | 0.0535 |
| 359 | | 0.0524 |
| 360 | | 0.0328 |
| 361 | | 0.0539 |
| 362 | | 0.0528 |
| 363 | | 0.0519 |
| 364 | | 0.0024 |
| 365 | | 0.0013 |
| 366 | | 0.0016 |
| 367 | | 0.0029 |
| 368 | | 0.0033 |
| 369 | | 0.0020 |
| 370 | | 0.0029 |
| 371 | | 0.0142 |
| 372 | | 0.0244 |
| 373 | | 0.0072 |
| 374 | | 0.0173 |
| 375 | | 0.0031 |
| 376 | | 0.0043 |
| 377 | | 0.0148 |
| 378 | | 0.0122 |
| 379 | | 0.0022 |
| 380 | | 0.0031 |
| 381 | | 0.0036 |
| 382 | | 0.0961 |
| 383 | | 0.0899 |
| 384 | | 0.1606 |
| 385 | | 0.1239 |
| 386 | | 0.1110 |
| 387 | | 0.0858 |
| 388 | | 0.1455 |
| 389 | | 0.1248 |
| 390 | | 0.0708 |
| 391 | | 0.0941 |
| 392 | | 0.0730 |
| 393 | | 0.0984 |
| 394 | | 0.1034 |
| 395 | | 0.0975 |
| 396 | | 0.0605 |
| 396 | | 0.0932 |
| 397 | | 0.1277 |
| 398 | | 0.0978 |
| 399 | | 0.1742 |
| 400 | | 0.1083 |
| 401 | | 0.1068 |
| 402 | | 0.0924 |
| 403 | | 0.0997 |
| 404 | | 0.1199 |
| 405 | | 0.0983 |
| 406 | | 0.1744 |
| 407 | | 0.0860 |
| 408 | | 0.0795 |
| 409 | | 0.1698 |
| 410 | | 0.0725 |
| 411 | | 0.0869 |
| 412 | | 0.1223 |
| 413 | | 0.1396 |
| 414 | | 0.0987 |
| 415 | | 0.1242 |
| 416 | | 0.0736 |
| 417 | | 0.1303 |
| 418 | | 0.1015 |
| 419 | | 0.1363 |
| 420 | | 0.1713 |
| 421 | | 0.1164 |
| 422 | | 0.0969 |
| 423 | | 0.1507 |
| 424 | | 0.0961 |
| 425 | | 0.1550 |
| 426 | | 0.0716 |
| 427 | | 0.0735 |
| 428 | | 0.1833 |
| 429 | | 0.0999 |
| 430 | | 0.0970 |
| 431 | | 0.1807 |
| 432 | | 0.0768 |
| 433 | | 0.1219 |
| 434 | | 0.1009 |
| 435 | | 0.0797 |
| 436 | | 0.0790 |
| 437 | | 0.0856 |
| 438 | | 0.0880 |
| 439 | | 0.1742 |
| 440 | | 0.0701 |
| 441 | | 0.0729 |
| 442 | | 0.1064 |
| 443 | | 0.0743 |
| 444 | | 0.1554 |
| 445 | | 0.0789 |
| 446 | | 0.1112 |
| 447 | | 0.0697 |
| 448 | | 0.1148 |
| 449 | | 0.0895 |
| 450 | | 0.0751 |
| 451 | | 0.1067 |
| 452 | | 0.1352 |
| 453 | | 0.1619 |
| 454 | | 0.0813 |
| 455 | | 0.1370 |
| 456 | | 0.1016 |
| 457 | | 0.1646 |
| 458 | | 0.1672 |
| 459 | | 0.1452 |
| 460 | | 0.1640 |
| 461 | | 0.0724 |
| 462 | | 0.0865 |
| 463 | | 0.0745 |
| 464 | | 0.0828 |
| 465 | | 0.0749 |
| 466 | | 0.0807 |
| 467 | | 0.1134 |
| 468 | | 0.0817 |
| 469 | | 0.0273 |
| 470 | | 0.1434 |
| 471 | | 0.0558 |
| 472 | | 0.1055 |
| 473 | | 0.1126 |
| 474 | | 0.4284 |
| 475 | | 0.0519 |
| 476 | | 0.0031 |
| 477 | | 0.0021 |
| 478 | | 0.0030 |
| 479 | | 0.0011 |
| 480 | | 0.0100 |
| 481 | | 0.0176 |
| 482 | | 0.0018 |
| 483 | | 0.0009 |
| 484 | | 0.0009 |
| 485 | | 0.0011 |
| 486 | 0.03969 | |
| 487 | 0.012909 | 0.3345 |
| 488 | 0.006450 | 0.4242 |
| 489 | | 13.6002 |
| 490 | | 7.3302 |
| 491 | | >50 |
| 492 | | 0.4315 |
| 493 | | 0.2364 |
| 494 | | 0.4236 |
| 495 | | 0.4816 |
| 496 | | 0.9949 |
| 497 | | 2.5258 |
| 498 | | 0.2319 |
| 499 | | 0.7202 |
| 500 | | 0.4307 |
| 501 | | 0.3553 |
| 502 | | 1.5811 |
| 503 | | 0.4419 |
| 504 | | 0.4664 |
| 505 | | 1.3837 |
| 506 | | 0.3498 |
| 507 | | 1.701 |

1. A compound of formula (II): 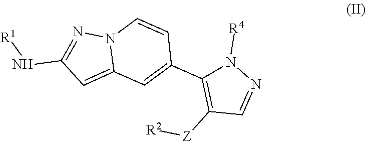
or a stereoisomer, tautomer, pharmaceutically acceptable salt or solvate thereof,
wherein:
Z is —O—(CH₂)ₙ—, provided that O is directly bonded to the pyrazolyl;
n is 0 or 1;
R⁴ is —CH₃, —CH₂CH₃, —CHF₂, —CD₃, —CH₂-cyclopropyl, or benzyl;
R¹ is —C(O)R⁵, phenyl, or a 5- to 6-membered heteroaryl having 1-2 nitrogen heteroatoms, or 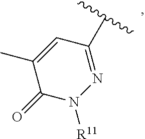
wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, —OCH₃, —CH₂-cyclopropyl-, —CHF₂, —COOCH₃, pyrazolyl, and pyridinyl, wherein the 5- to 6-membered heteroaryl is optionally fused to a ring selected from the group consisting of benzene, cyclopentane, tetrahydropyran, and a second 5- to 6-membered heteroaryl having 1-2 nitrogen heteroatoms, wherein the second 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, —CN, and phenyl;
R² is:
azetidinyl optionally substituted with one or more substituents independently selected from the group consisting of —CH₃, —CH₂CH₃, —CH(CH₃)₂, —CH₂CH(CH₃)₂, —CD₃, —CD₂CD₃, —F, —CF₃, —CH₂CH₂F, —CH₂CH₂OF₃, —CH₂CH(OH)CH₃, —CH₂C(OH)(CH₃)₂, —CH₂C(OCH₃)(CH₃)₂, —C(O)CH₃, —CH₂C(O)CH₃, —CH₂CH₂—CN, —CH₂C(CN)(CH₃)₂, pyridinyl, phenyl, —CH₂-cyclopropyl, —CH₂-phenyl, 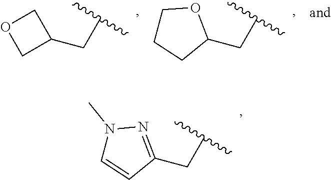
pyrrolidinyl optionally substituted with one or more substituents independently selected from the group consisting of —OCH₃, —CH₂CH₂—OCH₃, and —CH₃,
morpholinyl,
tetrahydrofuranyl optionally substituted with one or more —CH₃,
a 6- to 9-membered bicyclic heterocyclyl selected from the group consisting of 3-oxa-9-azabicyclo[3.3.1]nonanyl, 7-oxa-2-azaspiro[3.5]nonanyl, 5-azaspiro[2.3]hexanyl, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein the 6- to 9-membered bicyclic heterocyclyl is optionally substituted with one or more substituents independently selected from the group consisting of —CH₃ and —CH₂CH₃,
a C₁₋₅ alkyl optionally substituted with one or more substituents independently selected from the group consisting of —OH, —CH₃, —CF₃, —C₆H₅, 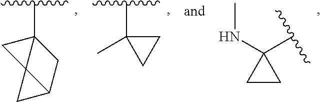
or
a C₃-C₆ cycloalkyl, wherein the C₃-C₆ cycloalkyl is optionally substituted with one or more —OH;
each R⁸ is independently hydrogen, cyclopropyl, or C₁₋₄ alkyl, wherein the C₁₋₄ alkyl is optionally substituted with one or more substituents independently selected from the group consisting of —CN, —OH, —OCH₃, and —F;
each R⁹ is independently C₁ fluoroalkyl or —CH₃;
each R¹⁰ is independently hydrogen, cyclopropyl, cyclopentyl, or —CH₃; and
each R¹¹ is independently hydrogen, C₁₋₅ alkyl, or —CH₂-cyclopropyl.

2. The compound of claim 1, wherein R⁴ is —CH₃.

3. The compound of claim 1, wherein Z is —O—.

4. The compound of claim 1, wherein R² is azetidinyl optionally substituted with one or more substituents independently selected from the group consisting of —CH₃, —CH₂CH₃, —CH(CH₃)₂, —CH₂CH(CH₃)₂, —CD₃, —CD₂CD₃, —F, —CF₃, —CH₂CH₂F, —CH₂CH₂OF₃, —CH₂CH(OH)CH₃, —CH₂C(OH)(CH₃)₂, —CH₂C(OCH₃)(CH₃)₂, —C(O)CH₃, —CH₂C(O)CH₃, —CH₂CH₂—CN, —CH₂C(CN)(CH₃)₂, pyridinyl, phenyl, —CH₂-cyclopropyl, —CH₂-phenyl, 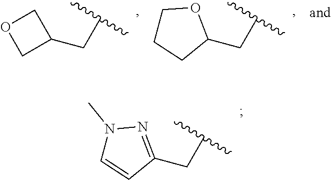
pyrrolidinyl optionally substituted with one or more substituents independently selected from the group consisting of —OCH₃, —CH₂CH₂—OCH₃, and —CH₃; morpholinyl; or tetrahydrofuranyl optionally substituted with one or more —CH₃.

5. The compound of claim 1, wherein R⁸ is a 6- to 9-membered bicyclic heterocyclyl selected from the group consisting of 3-oxa-9-azabicyclo[3.3.1]nonanyl, 7-oxa-2-azaspiro[3.5]nonanyl, 5-azaspiro[2.3]hexanyl, 5-oxa-2-azabicyclo[2.2.1]heptanyl, 3-azabicyclo[2.1.1]hexanyl, 6,9-dioxaspiro[4.4]nonanyl, 7-oxabicyclo[2.2.1]heptanyl, and 3-azabicyclo[3.1.0]hexanyl, wherein the 6- to 9-membered bicyclic heterocyclyl is optionally substituted with one or more substituents independently selected from the group consisting of —CH₃ and —CH₂CH₃.

6. The compound of claim 1, wherein R² is a C₁₋₅ alkyl optionally substituted with one or more substituents independently selected from the group consisting of —OH, —CH₃, —CF₃, —C₆H₅,

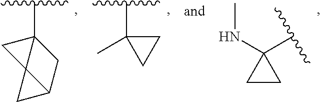

7. The compound of claim 1, wherein R¹ is —C(O)R⁵ and R⁵ is cyclopropyl optionally substituted with one or more substituents independently selected from the group consisting of —CH₃, —F, —CF₃, —CN, and phenyl; or R¹ is pyrazinyl, pyridinyl, pyridazinyl, pyrimidinyl, or pyrazolyl, wherein each of the pyrazinyl, pyridinyl, pyridazinyl, pyrimidinyl, or pyrazolyl is optionally substituted with one or more substituents independently selected from the group consisting of —CH₃, cyclopropyl, CF₃, —CH₂-cyclopropyl, —OCHF₂, —COOCH₃, pyridinyl, and

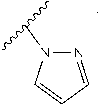 [SMILES: *n1cccn1]

8. A compound of formula (III): 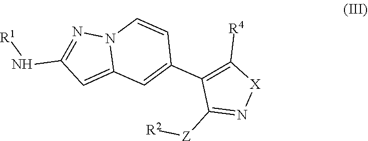
or a stereoisomer, tautomer, pharmaceutically acceptable salt or solvate thereof, wherein:
X is O or S;
Z is —O—(CH₂)ₙ—, provided that O is directly bonded to the isoxazolyl or isothiazolyl;
n is 0 or 1;
R⁴ is —CH₃ or —CH₂CH₃;
R¹ is —C(O)R⁵, phenyl, a 5- to 6-membered heteroaryl having 1-2 nitrogen heteroatoms, or 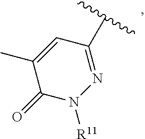
wherein each of the phenyl and 5- to 6-membered heteroaryl is optionally substituted with one or more substituents independently selected from the group consisting of C₁-C₄ alkyl, —F, C₁ fluoroalkyl, C₁₋₄ hydroxyalkyl, —CON(R⁸)₂, —C(O)R⁹, cyclopropyl, —CH₂OCH₃, —N(R¹⁰)₂, and —OCH₃, wherein the 5- to 6-membered heteroaryl is optionally fused to a second 5- to 6-membered heteroaryl having 1-2 nitrogen heteroatoms, wherein the 5- to 6-membered heteroaryl is optionally substituted with one or more —CH₃;
R⁵ is cyclopropyl optionally substituted with one or more substituents independently selected from the group consisting of —F, —CH₃, C₁ fluoroalkyl, and phenyl;
R² is:
a 4- to 7-membered monocyclic heterocyclyl selected from the group consisting of pyrrolidinyl, morpholinyl, and azetidinyl, wherein each of the 4- to 7-membered monocyclic heterocyclyl is optionally substituted with one or more substituents independently selected from the group consisting of —CH₃, —OCH₃, —CH₂CH₃, —CH(CH₃)₂, —CH₂CF₃, —CHF₂, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl,
a 6- to 9-membered bicyclic heterocyclyl selected from the group consisting of 2-oxa-5-azabicyclo[2.2.1]heptanyl, 5-oxa-2-azabicyclo[2.2.1]heptanyl, 2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 6- to 9-membered bicyclic heterocyclyl is optionally substituted with one or more substituents independently selected from the group consisting of —CH₃ and —CH₂CH₃, or
a C₁₋₅ alkyl optionally substituted with one or more substituents independently selected from the group consisting of —NH₂ phenyl, and fluorophenyl; or
a C₃-C₆ cycloalkyl, wherein the C₃-C₆ cycloalkyl is optionally substituted with one or more —NH₂;
each R⁸ is independently hydrogen, cyclopropyl, or C₁₋₄ alkyl, wherein the C₁₋₄ alkyl is optionally substituted with one or more —F;
each R⁹ is independently C₁ fluoroalkyl or —CH₃;
each R¹⁰ is independently hydrogen or —CH₃; and
each R¹¹ is independently hydrogen, C₁₋₅ alkyl, or —CH₂-cyclopropyl.

9. The compound of claim 8, wherein X is O.

10. The compound of claim 8, wherein R¹ is —C(O)R⁵ and R⁵ is cyclopropyl.

11. The compound of claim 8, wherein R¹ is pyridazinyl or pyrimidinyl, wherein each of the pyridazinyl and pyrimidinyl is optionally substituted with one or more substituents independently selected from the group consisting of —C(O)NCH₃, —F, —CH₃, —CHF₂, —C(O)CF₃, —CH₂C(OH)(CH₃)₂, —CH(CH₃)₂, and —CH₂OCH₃.

12. The compound of claim 1, wherein R² is a 4- to 7-membered monocyclic heterocyclyl selected from the group consisting of pyrrolidinyl, morpholinyl, and azetidinyl, wherein each of the 4- to 7-membered monocyclic heterocyclyl is optionally substituted with one or more substituents independently selected from the group consisting of —CH₃, —OCH₃, —CH₂CH₃, —CH(CH₃)₂, —CH₂CF₃, —CHF₂, —CF₃, —F, —OH, —NH₂, —CN, oxo, cyclopropyl, —COOC(CH₃)₃, —C(O)CH₃, —CH₂CN, —CH₂CH₂OCH₃, —CH₂(C₅H₄N), —CD₃, —CH₂—OCH₃, —CONH₂, and phenyl.

13. The compound of claim 1, wherein R² is a 6- to 9-membered bicyclic heterocyclyl selected from the group consisting of 2-oxa-5-azabicyclo[2.2.1]heptanyl, 5-oxa-2-azabicyclo[2.2.1]heptanyl, 2-azabicyclo[2.2.1]heptanyl, 9-azaspiro[3.5]nonanyl, and 3-(1,2,3,3a,5,6,7,7a-octahydropyrano[3,2-b]pyrrolyl, wherein each of the 6- to 9-membered bicyclic heterocyclyl is optionally substituted with one or more substituents independently selected from the group consisting of —CH₃ and —CH₂CH₃.

14. The compound of claim 1, wherein R² is a C₁₋₅ alkyl optionally substituted with one or more substituents independently selected from the group consisting of —NH₂ phenyl, and fluorophenyl.

15. A compound selected from the group consisting of:
N-(5-(4-(((3S,5S)-1,5-dimethylpyrrolidin-3-yl)oxy)-1-methyl-1H-pyrazol-5-yl)pyrazolo[1,5-a]pyridin-2-yl)cyclopropanecarboxamide;
N-(2,6-dimethylpyrimidin-4-yl)-5-[2-methyl-4-[[(2R)-1-methylazetidin-2-yl]methoxy]pyrazol-3-yl]pyrazolo[1,5-a]pyridin-2-amine;
N-[5-[2-methyl-4-[[(2S)-1,3,3-trimethylazetidin-2-yl]methoxy]pyrazol-3-yl]pyrazolo[1,5-a]pyridin-2-yl]cyclopropanecarboxamide;
N-[5-[4-[[(2S)-3,3-dimethylazetidin-2-yl]methoxy]-2-methyl-pyrazol-3-yl]pyrazolo[1,5-a]pyridin-2-yl]cyclopropanecarboxamide;
N-(5-(4-(((3*R,4*S)-4-methoxypyrrolidin-3-yl)oxy)-1-methyl-1H-pyrazol-5-yl)pyrazolo[1,5-a]pyridin-2-yl)cyclopropanecarboxamide;
N-[5-[2-methyl-4-[[(2R)-morpholin-2-yl]methoxy]pyrazol-3-yl]pyrazolo[1,5-a]pyridin-2-yl]cyclopropanecarboxamide;
5-[2-methyl-4-[(3S)-1-methylpyrrolidin-3-yl]oxy-pyrazol-3-yl]-N-[6-(trifluoromethyl)pyrazin-2-yl]pyrazolo[1,5-a]pyridin-2-amine;
N-[5-[2-methyl-4-[(1-methylazetidin-3-yl)methoxy]pyrazol-3-yl]pyrazolo[1,5-a]pyridin-2-yl]cyclopropanecarboxamide;
N-[5-[2-methyl-4-[[(2S)-1-methylazetidin-2-yl]methoxy]pyrazol-3-yl]pyrazolo[1,5-a]pyridin-2-yl]cyclopropanecarboxamide;
N-[5-[2-methyl-4-[[(2R,3R)-1-methyl-3-(trifluoromethyl)azetidin-2-yl]methoxy]pyrazol-3-yl]pyrazolo[1,5-a]pyridin-2-yl]cyclopropanecarboxamide;
N-[5-[4-[[(2S,3R)-3-ethyl-1-methyl-azetidin-2-yl]methoxy]-2-methyl-pyrazol-3-yl]pyrazolo[1,5-a]pyridin-2-yl]cyclopropanecarboxamide;
N-[5-[4-[[(2S,3S)-1,3-dimethylazetidin-2-yl]methoxy]-2-methyl-pyrazol-3-yl]pyrazolo[1,5-a]pyridin-2-yl]cyclopropanecarboxamide;
(rac)-N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide;
(1R,2R)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide;
(1S,2S)-2-methyl-N-[5-[2-methyl-4-[[(2R)-1-methylazetidin-2-yl]methoxy]pyrazol-3-yl]pyrazolo[1,5-a]pyridin-2-yl]cyclopropanecarboxamide;
(*R)—N-(5-(4-((5-Ethyl-5-azaspiro[2.3]hexan-4-yl)methoxy)-1-methyl-1H-pyrazol-5-yl)pyrazolo[1,5-a]pyridin-2-yl)cyclopropanecarboxamide;
N-(5,6-Dimethylpyridazin-3-yl)-5-[2-methyl-4-[[(2R)-1-methylazetidin-2-yl]methoxy]pyrazol-3-yl]pyrazolo[1,5-a]pyridin-2-amine;
(R)—N-(5,6-Dimethylpyridazin-3-yl)-5-(4-((1-ethylazetidin-2-yl)methoxy)-1-methyl-1H-pyrazol-5-yl)pyrazolo[1,5-a]pyridin-2-amine;
5-[4-[[(2R)-1-Ethylazetidin-2-yl]methoxy]-2-methyl-pyrazol-3-yl]-N-(6-methylpyrimidin-4-yl)pyrazolo[1,5-a]pyridin-2-amine;
(R)-5-(4-((1-Ethylazetidin-2-yl)methoxy)-1-methyl-1H-pyrazol-5-yl)-N-(6-methylpyrazin-2-yl)pyrazolo[1,5-a]pyridin-2-amine;
(R)-5-(4-((1-Ethylazetidin-2-yl)methoxy)-1-methyl-1H-pyrazol-5-yl)-N-(5-methylpyrazin-2-yl)pyrazolo[1,5-a]pyridin-2-amine;
(R)—N-(5,6-Dimethylpyrazin-2-yl)-5-(4-((1-ethylazetidin-2-yl)methoxy)-1-methyl-1H-pyrazol-5-yl)pyrazolo[1,5-a]pyridin-2-amine;
N-(5,6-Dimethylpyrazin-2-yl)-5-[2-methyl-4-[[(2R)-1-methylazetidin-2-yl]methoxy]pyrazol-3-yl]pyrazolo[1,5-a]pyridin-2-amine;
N-[5-[4-[[(2R,3R)-1,3-Dimethylazetidin-2-yl]methoxy]-2-methyl-pyrazol-3-yl]pyrazolo[1,5-a]pyridin-2-yl]cyclopropanecarboxamide;
(1S,2S)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide;
1-methyl-N-[5-[2-methyl-4-[[(2R)-1-methylazetidin-2-yl]methoxy]pyrazol-3-yl]pyrazolo[1,5-a]pyridin-2-yl]pyrazole-4-carboxamide;
N-[5-[4-[(2S)-2-hydroxy-2-phenyl-ethoxy]-2-methyl-pyrazol-3-yl]pyrazolo[1,5-a]pyridin-2-yl]cyclopropanecarboxamide;
N-[5-[5-methyl-3-[[(2S)-morpholin-2-yl]methoxy]isoxazol-4-yl]pyrazolo[1,5-a]pyridin-2-yl]cyclopropanecarboxamide;
N-[5-[3-[(3-methoxy-1-methyl-azetidin-3-yl)methoxy]-5-methyl-isoxazol-4-yl]pyrazolo[1,5-a]pyridin-2-yl]cyclopropanecarboxamide;
N-(2,6-dimethylpyrimidin-4-yl)-5-[3-[(3-methoxyazetidin-3-yl)methoxy]-5-methyl-isoxazol-4-yl]pyrazolo[1,5-a]pyridin-2-amine;
N-[5-[3-[(2S)-2-amino-2-phenyl-ethoxy]-5-methyl-isoxazol-4-yl]pyrazolo[1,5-a]pyridin-2-yl]cyclopropanecarboxamide;
5-[2-(difluoromethyl)-4-[(3R,4S)-4-fluoropyrrolidin-3-yl]oxy-pyrazol-3-yl]-N-(2,6-dimethylpyrimidin-4-yl)pyrazolo[1,5-a]pyridin-2-amine;
6-[[5-[2-(difluoromethyl)-4-[[(2R)-1-methylazetidin-2-yl]methoxy]pyrazol-3-yl]pyrazolo[1,5-a]pyridin-2-yl]amino]-2,4-dimethyl-pyridazin-3-one;
(*S)-3-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one;
*R-4-[[5-[2-[(2,6-dimethylpyrimidin-4-yl)amino]pyrazolo[1,5-a]pyridin-5-yl]-1-methyl-pyrazol-4-yl]oxymethyl]-1-methyl-pyrrolidin-2-one;
N-(2-cyano-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-isopropyl-2,6-dimethoxy-4-[[5-[2-methyl-4-[[(2R)-1-methylazetidin-2-yl]methoxy]pyrazol-3-yl]pyrazolo[1,5-a]pyridin-2-yl]amino]benzamide;
N-[5-[2-methyl-4-[[(2R)-1-methylazetidin-2-yl]methoxy]pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(6-methylpyridazin-3-yl)pyrazolo[1,5-a]pyridin-2-amine;
N-(5-(4-(((3*S,4*R)-4-methoxypyrrolidin-3-yl)oxy)-1-methyl-1H-pyrazol-5-yl)pyrazolo[1,5-a]pyridin-2-yl)cyclopropanecarboxamide;
trans-N-(5-(4-(((2*R,3*S)-1,2-dimethylazetidin-3-yl)methoxy)-1-methyl-1H-pyrazol-5-yl)pyrazolo[1,5-a]pyridin-2-yl)cyclopropanecarboxamide;
5-[4-[[(2R)-azetidin-2-yl]methoxy]-2-methyl-pyrazol-3-yl]-N-(2,6-dimethylpyrimidin-4-yl)pyrazolo[1,5-a]pyridin-2-amine;
exo-N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide;
N-[5-[2-methyl-4-[(3-phenylazetidin-3-yl)methoxy]pyrazol-3-yl]pyrazolo[1,5-a]pyridin-2-yl]cyclopropanecarboxamide;
5-[4-[[(2R)-azetidin-2-yl]methoxy]-2-methyl-pyrazol-3-yl]-N-(6-methylpyrazin-2-yl)pyrazolo[1,5-a]pyridin-2-amine;
5-[2-methyl-4-[[(2R)-1-methylazetidin-2-yl]methoxy]pyrazol-3-yl]-N-(6-methylpyrazin-2-yl)pyrazolo[1,5-a]pyridin-2-amine;
N-[5-[4-[[(2S)-1-ethylazetidin-2-yl]methoxy]-2-methyl-pyrazol-3-yl]pyrazolo[1,5-a]pyridin-2-yl]cyclopropanecarboxamide;
N-[5-[4-[[(2S)-1-isopropylazetidin-2-yl]methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(5-methylpyrazin-2-yl)pyrazolo[1,5-a]pyridin-2-amine;
N-(6-cyclopropylpyridazin-3-yl)-5-[2-methyl-4-[[(2R)-1-methylazetidin-2-yl]methoxy]pyrazol-3-yl]pyrazolo[1,5-a]pyridin-2-amine;
(rac)-N-[5-[2-methyl-4-[(5-methyl-5-azaspiro[2.3]hexan-6-yl)methoxy]pyrazol-3-yl]pyrazolo[1,5-a]pyridin-2-yl]cyclopropanecarboxamide;
N-[5-[4-[[(2R)-1-ethylazetidin-2-yl]methoxy]-2-methyl-pyrazol-3-yl]pyrazolo[1,5-a]pyridin-2-yl]cyclopropanecarboxamide;
N-[5-[4-[[(2R)-1-isopropylazetidin-2-yl]methoxy]-2-methyl-pyrazol-3-yl]pyrazolo[1,5-a]pyridin-2-yl]cyclopropanecarboxamide;
(rac)-N-[5-[4-[(1,2-dimethylazetidin-2-yl)methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(6-methylpyrimidin-4-yl)pyrazolo[1,5-a]pyridin-2-amine;
N-[5-[4-[[(2R)-1-acetylazetidin-2-yl]methoxy]-2-methyl-pyrazol-3-yl]pyrazolo[1,5-a]pyridin-2-yl]cyclopropanecarboxamide;
5-[2-methyl-4-[[(2R)-1-methylazetidin-2-yl]methoxy]pyrazol-3-yl]-N-(2,5,6-trimethylpyrimidin-4-yl)pyrazolo[1,5-a]pyridin-2-amine;
(*R)—N-(5-(1-methyl-4-((2-methyl-7-oxa-2-azaspiro[3.5]nonan-1-yl)methoxy)-1H-pyrazol-5-yl) pyrazolo[1,5-a]pyridin-2-yl)cyclopropanecarboxamide;
(*S)—N-[5-[2-methyl-4-[(2-methyl-7-oxa-2-azaspiro[3.5]nonan-3-yl)methoxy]pyrazol-3-yl]pyrazolo[1,5-a]pyridin-2-yl]cyclopropanecarboxamide;
rac-N-[5-[4-[(3,3-difluoro-1-methyl-azetidin-2-yl)methoxy]-2-methyl-pyrazol-3-yl]pyrazolo[1,5-a]pyridin-2-yl]cyclopropanecarboxamide;
N-(4,6-dimethylpyridazin-3-yl)-5-[2-methyl-4-[[(2R)-1-methylazetidin-2-yl]methoxy]pyrazol-3-yl]pyrazolo[1,5-a]pyridin-2-amine;
N-(2,6-dimethylpyrimidin-4-yl)-5-[4-[[(2R)-1-ethylazetidin-2-yl]methoxy]-2-methyl-pyrazol-3-yl]pyrazolo[1,5-a]pyridin-2-amine;
5-[2-methyl-4-[[(2R)-1-methylazetidin-2-yl]methoxy]pyrazol-3-yl]-N-[6-methyl-2-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-(2-methylpyrimidin-4-yl)pyrazolo[1,5-a]pyridin-2-amine;
5-[4-[[(2R)-1-ethylazetidin-2-yl]methoxy]-2-methyl-pyrazol-3-yl]-N-(2-methylpyrimidin-4-yl)pyrazolo[1,5-a]pyridin-2-amine;
(1R,2R)—N-[5-[4-[[(2R)-1-ethylazetidin-2-yl]methoxy]-2-methyl-pyrazol-3-yl]pyrazolo[1,5-a]pyridin-2-yl]-2-methyl-cyclopropanecarboxamide;
(*S)—N-[5-[4-[(5-ethyl-5-azaspiro[2.3]hexan-6-yl)methoxy]-2-methyl-pyrazol-3-yl]pyrazolo[1,5-a]pyridin-2-yl]cyclopropanecarboxamide;
5-[4-[[(2R)-1-ethylazetidin-2-yl]methoxy]-2-methyl-pyrazol-3-yl]-N-(6-methylpyridazin-3-yl)pyrazolo[1,5-a]pyridin-2-amine;
(1S,2S)-2-fluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide;
trans-N-[5-[2-methyl-4-[((2R)-1-methylazetidin-2-yl)methoxy]pyrazol-3-yl]pyrazolo[1,5-a]pyridin-2-yl]-2-(trifluoromethyl)cyclopropanecarboxamide;
trans-N-[5-[2-methyl-4-[((2R)-1-methylazetidin-2-yl)methoxy]pyrazol-3-yl]pyrazolo[1,5-a]pyridin-2-yl]-2-phenyl-cyclopropanecarboxamide;
2,2-difluoro-N-[5-[2-methyl-4-[[(2R)-1-methylazetidin-2-yl]methoxy]pyrazol-3-yl]pyrazolo[1,5-a]pyridin-2-yl]cyclopropanecarboxamide;
5-[4-[[(2R)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
N-[5-[5-methyl-3-[(3R)-pyrrolidin-3-yl]oxy-isoxazol-4-yl]pyrazolo[1,5-a]pyridin-2-yl]cyclopropanecarboxamide;
N-[5-[5-methyl-3-[(3S)-pyrrolidin-3-yl]oxy-isoxazol-4-yl]pyrazolo[1,5-a]pyridin-2-yl]cyclopropanecarboxamide;
N-[5-[3-[(3-methoxyazetidin-3-yl)methoxy]-5-methyl-isoxazol-4-yl]pyrazolo[1,5-a]pyridin-2-yl]cyclopropanecarboxamide;
N-[5-[3-[(3-cyanoazetidin-3-yl)methoxy]-5-methyl-isoxazol-4-yl]pyrazolo[1,5-a]pyridin-2-yl]cyclopropanecarboxamide;
N-[5-[5-methyl-3-[[(2R)-morpholin-2-yl]methoxy]isoxazol-4-yl]pyrazolo[1,5-a]pyridin-2-yl]cyclopropanecarboxamide;
N-[5-[5-methyl-3-[[(2R)-4-methylmorpholin-2-yl]methoxy]isoxazol-4-yl]pyrazolo[1,5-a]pyridin-2-yl]cyclopropanecarboxamide;
N-[5-[3-[(3S,4R)-4-fluoropyrrolidin-3-yl]oxy-5-methyl-isoxazol-4-yl]pyrazolo[1,5-a]pyridin-2-yl]cyclopropanecarboxamide;
N-[5-[5-methyl-3-[[(1S,4S)-5-oxa-2-azabicyclo[2.2.1]heptan-4-yl]methoxy]isoxazol-4-yl]pyrazolo[1,5-a]pyridin-2-yl]cyclopropanecarboxamide;
N-[5-[3-[(3S,4S)-4-fluoropyrrolidin-3-yl]oxy-5-methyl-isoxazol-4-yl]pyrazolo[1,5-a]pyridin-2-yl]cyclopropanecarboxamide;
N-[5-[3-[(3-fluoroazetidin-3-yl)methoxy]-5-methyl-isoxazol-4-yl]pyrazolo[1,5-a]pyridin-2-yl]cyclopropanecarboxamide;
N-[5-[3-[(3-fluoro-1-methyl-azetidin-3-yl)methoxy]-5-methyl-isoxazol-4-yl]pyrazolo[1,5-a]pyridin-2-yl]cyclopropanecarboxamide;
N-[5-[5-methyl-3-[(3S,5R)-5-methylpyrrolidin-3-yl]oxy-isoxazol-4-yl]pyrazolo[1,5-a]pyridin-2-yl]cyclopropanecarboxamide;
N-[5-[3-[[(2R)-6,6-dimethylmorpholin-2-yl]methoxy]-5-methyl-isoxazol-4-yl]pyrazolo[1,5-a]pyridin-2-yl]cyclopropanecarboxamide;
N-[5-[5-methyl-3-[[(2R)-1-methylazetidin-2-yl]methoxy]isoxazol-4-yl]pyrazolo[1,5-a]pyridin-2-yl]cyclopropanecarboxamide;
N-[5-[3-[(3R,4S)-4-methoxypyrrolidin-3-yl]oxy-5-methyl-isoxazol-4-yl]pyrazolo[1,5-a]pyridin-2-yl]cyclopropanecarboxamide;
N-[5-[3-[[(2S)-3,3-dimethylazetidin-2-yl]methoxy]-5-methyl-isoxazol-4-yl]pyrazolo[1,5-a]pyridin-2-yl]cyclopropanecarboxamide;
N-(2,6-dimethylpyrimidin-4-yl)-5-[5-methyl-3-[(3S,5R)-5-methylpyrrolidin-3-yl]oxy-isoxazol-4-yl]pyrazolo[1,5-a]pyridin-2-amine;
N-(2,6-dimethylpyrimidin-4-yl)-5-[5-methyl-3-[[(2R)-1-methylazetidin-2-yl]methoxy]isoxazol-4-yl]pyrazolo[1,5-a]pyridin-2-amine;
N-(2,6-dimethylpyrimidin-4-yl)-5-[3-[(3R,4S)-4-fluoropyrrolidin-3-yl]oxy-5-methyl-isoxazol-4-yl]pyrazolo[1,5-a]pyridin-2-amine;
N-(5,6-dimethylpyridazin-3-yl)-5-[3-[(3R,4S)-4-fluoropyrrolidin-3-yl]oxy-5-methyl-isoxazol-4-yl]pyrazolo[1,5-a]pyridin-2-amine;
N-(2,6-dimethylpyrimidin-4-yl)-5-[3-[(3R,4S)-4-methoxypyrrolidin-3-yl]oxy-5-methyl-isoxazol-4-yl]pyrazolo[1,5-a]pyridin-2-amine;
N-(5,6-dimethylpyridazin-3-yl)-5-[3-[(3R,4S)-4-methoxypyrrolidin-3-yl]oxy-5-methyl-isoxazol-4-yl]pyrazolo[1,5-a]pyridin-2-amine;
5-[2-(difluoromethyl)-4-[(3R,4S)-4-methoxypyrrolidin-3-yl]oxy-pyrazol-3-yl]-N-(2,6-dimethylpyrimidin-4-yl)pyrazolo[1,5-a]pyridin-2-amine;
5-[2-(difluoromethyl)-4-[[(2R)-1-methylazetidin-2-yl]methoxy]pyrazol-3-yl]-N-(6-methoxy-5-m ethyl-pyridazin-3-yl)pyrazolo[1,5-a]pyridin-2-amine;
5-[2-(difluoromethyl)-4-[[(2R)-1-methylazetidin-2-yl]methoxy]pyrazol-3-yl]-N-(6-methylpyridazin-3-yl)pyrazolo[1,5-a]pyridin-2-amine;
(2R,3S)-2-[[1-(difluoromethyl)-5-[2-[(2,6-dimethylpyrimidin-4-yl)amino]pyrazolo[1,5-a]pyridin-5-yl]pyrazol-4-yl]oxymethyl]tetrahydrofuran-3-ol;
(RS)-5-[2-(difluoromethyl)-4-[(3-methoxypyrrolidin-3-yl)methoxy]pyrazol-3-yl]-N-(2,6-dimethylpyrimidin-4-yl)pyrazolo[1,5-a]pyridin-2-amine;
(RS)-5-[2-(difluoromethyl)-4-[(3-fluoropyrrolidin-3-yl)methoxy]pyrazol-3-yl]-N-(2,6-dimethylpyrimidin-4-yl)pyrazolo[1,5-a]pyridin-2-amine;
N-[2-(difluoromethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine;
N-[2-(difluoromethyl)-6-methyl-pyrimidin-4-yl]-5-[4-[[(2R)-1-ethylazetidin-2-yl]methoxy]-2-m ethyl-pyrazol-3-yl]pyrazolo[1,5-a]pyridin-2-amine;
5-[2-(difluoromethyl)-4-[[(2R)-1-ethylazetidin-2-yl]methoxy]pyrazol-3-yl]-N-(1-methylpyrazol-4-yl)pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]cyclopropanecarboxamide;
N-(2-hydroxy-2-methyl-propyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(2-methoxyethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N,N,2-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide;
N,4-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide;
N,N,4-trimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-2-carboxamide;
N-(2-hydroxy-2-methyl-propyl)-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide;
N-isopropyl-2-methyl-6-[[5-[2-methyl-4-[[(2R)-1-methylazetidin-2-yl]methoxy]pyrazol-3-yl]pyrazolo[1,5-a]pyridin-2-yl]amino]pyridine-4-carboxamide;
N-(2-hydroxy-2-methyl-propyl)-4-methyl-6-[[5-[2-methyl-4-[[(2R)-1-methylazetidin-2-yl]methoxy]pyrazol-3-yl]pyrazolo[1,5-a]pyridin-2-yl]amino]pyridine-2-carboxamide;
N-[5-[2-methyl-4-[[(2R)-1-methylazetidin-2-yl]methoxy]pyrazol-3-yl]pyrazolo[1,5-a]pyridin-2-yl]-7,8-dihydro-5H-pyrano[4,3-b]pyridin-2-amine;
N-(2-methoxyethyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
5-[2-methyl-4-[[(2R)-1-methylazetidin-2-yl]methoxy]pyrazol-3-yl]-N-[6-methyl-2-(3-pyridyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine;
5-[2-methyl-4-[[(2R)-1-methylazetidin-2-yl]methoxy]pyrazol-3-yl]-N-[2-(trifluoromethyl)pyrimidin-4-yl]pyrazolo[1,5-a]pyridin-2-amine;
N-(2-methoxypyrimidin-4-yl)-5-[2-methyl-4-[[(2R)-1-methylazetidin-2-yl]methoxy]pyrazol-3-yl]pyrazolo[1,5-a]pyridin-2-amine;
N,2-dimethyl-6-[[5-[2-methyl-4-[[(2R)-1-methylazetidin-2-yl]methoxy]pyrazol-3-yl]pyrazolo[1,5-a]pyridin-2-yl]amino]pyrimidine-4-carboxamide;
N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine;
5-[2-methyl-4-[[(2R)-1-methylazetidin-2-yl]methoxy]pyrazol-3-yl]-N-[2-methyl-6-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-[6-(trifluoromethyl)pyrimidin-4-yl]pyrazolo[1,5-a]pyridin-2-amine;
5-[2-methyl-4-[[(2R)-1-methylazetidin-2-yl]methoxy]pyrazol-3-yl]-N-(1-methyltriazol-4-yl)pyrazolo[1,5-a]pyridin-2-amine;
N-[5-[2-methyl-4-[[(2R)-1-methylazetidin-2-yl]methoxy]pyrazol-3-yl]pyrazolo[1,5-a]pyridin-2-yl]-1,6-naphthyridin-2-amine;
N-methyl-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(6-methoxypyrimidin-4-yl)-5-[2-methyl-4-[[(2R)-1-methylazetidin-2-yl]methoxy]pyrazol-3-yl]pyrazolo[1,5-a]pyridin-2-amine;
N2-cyclopropyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine;
N2-cyclopentyl-6-methyl-N4-[5-[2-methyl-4-[[(2R)-1-methylazetidin-2-yl]methoxy]pyrazol-3-yl]pyrazolo[1,5-a]pyridin-2-yl]pyrimidine-2,4-diamine;
N-(2,6-dimethylpyrimidin-4-yl)-5-[2-ethyl-4-[[(2R)-1-methylazetidin-2-yl]methoxy]pyrazol-3-yl]pyrazolo[1,5-a]pyridin-2-amine;
N-(2,6-dimethylpyrimidin-4-yl)-5-[4-[[(2R)-1-methylazetidin-2-yl]methoxy]-2-(trideuteriomethyl)pyrazol-3-yl]pyrazolo[1,5-a]pyridin-2-amine;
N-(2-hydroxy-2-methyl-propyl)-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
N-methyl-5-[[5-[2-methyl-4-[[(2R)-1-methylazetidin-2-yl]methoxy]pyrazol-3-yl]pyrazolo[1,5-a]pyridin-2-yl]amino]pyrazine-2-carboxamide;
N-(2-cyclopropyl-6-methyl-pyrimidin-4-yl)-5-[2-methyl-4-[[(2S)-1-methylazetidin-2-yl]methoxy]pyrazol-3-yl]pyrazolo[1,5-a]pyridin-2-amine;
N-(2-cyanoethyl)-6-[[5-[2-methyl-4-[[(2R)-1-methylazetidin-2-yl]methoxy]pyrazol-3-yl]pyrazolo[1,5-a]pyridin-2-yl]amino]pyridine-3-carboxamide;
N-(4-methyl-6,7-dihydro-5H-cyclopenta[b]pyridin-2-yl)-5-[2-methyl-4-[[(2R)-1-methylazetidin-2-yl]methoxy]pyrazol-3-yl]pyrazolo[1,5-a]pyridin-2-amine;
N-(6-ethyl-2-methyl-pyrimidin-4-yl)-5-[2-methyl-4-[[(2S)-1-methylazetidin-2-yl]methoxy]pyrazol-3-yl]pyrazolo[1,5-a]pyridin-2-amine;
N-(5,6-dimethylpyrazin-2-yl)-5-[2-methyl-4-[[(2S)-1-methylazetidin-2-yl]methoxy]pyrazol-3-yl]pyrazolo[1,5-a]pyridin-2-amine;
N-(2,6-dimethylpyrimidin-4-yl)-5-[2-methyl-4-[[(2S)-1-methylazetidin-2-yl]methoxy]pyrazol-3-yl]pyrazolo[1,5-a]pyridin-2-amine;
5-[2-(difluoromethyl)-4-[[(2R)-1-methylazetidin-2-yl]methoxy]pyrazol-3-yl]-N-(2,6-dimethylpyrimidin-4-yl)pyrazolo[1,5-a]pyridin-2-amine;
5-[2-methyl-4-[[(2R)-1-methylazetidin-2-yl]methoxy]pyrazol-3-yl]-N-(5-methylpyridazin-3-yl)pyrazolo[1,5-a]pyridin-2-amine;
5-[2-methyl-4-[[(2R)-1-methylazetidin-2-yl]methoxy]pyrazol-3-yl]-N-(1-methylpyrazol-4-yl)pyrazolo[1,5-a]pyridin-2-amine;
2-methyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one;
N-(2-cyclopropyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(6-ethyl-2-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(2-methoxy-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
N-(6-methoxy-5-methyl-pyridazin-3-yl)-5-[2-methyl-4-[[(2R)-1-methylazetidin-2-yl]methoxy]pyrazol-3-yl]pyrazolo[1,5-a]pyridin-2-amine;
2-methyl-N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine;
N-[5-[2-methyl-4-[[(2R)-1-methylazetidin-2-yl]methoxy]pyrazol-3-yl]pyrazolo[1,5-a]pyridin-2-yl]imidazo[1,2-b]pyridazin-6-amine;
5-[2-methyl-4-[[(2R)-1-methylazetidin-2-yl]methoxy]pyrazol-3-yl]-N-[5-(trifluoromethyl)pyrazin-2-yl]pyrazolo[1,5-a]pyridin-2-amine;
N-(2-tert-butyl-6-methyl-pyrimidin-4-yl)-5-[2-methyl-4-[[(2R)-1-methylazetidin-2-yl]methoxy]pyrazol-3-yl]pyrazolo[1,5-a]pyridin-2-amine;
2,4-dimethyl-6-[[5-[2-methyl-4-[[(2R)-1-methylazetidin-2-yl]methoxy]pyrazol-3-yl]pyrazolo[1,5-a]pyridin-2-yl]amino]pyridazin-3-one;
N-[2-(cyclopropylmethyl)-6-methyl-pyrimidin-4-yl]-5-[2-methyl-4-[[(2R)-1-methylazetidin-2-yl]methoxy]pyrazol-3-yl]pyrazolo[1,5-a]pyridin-2-amine;
N-(5-methoxypyrazin-2-yl)-5-[2-methyl-4-[[(2R)-1-methylazetidin-2-yl]methoxy]pyrazol-3-yl]pyrazolo[1,5-a]pyridin-2-amine;
N-[5-(difluoromethoxy)pyrazin-2-yl]-5-[2-methyl-4-[[(2R)-1-methylazetidin-2-yl]methoxy]pyrazol-3-yl]pyrazolo[1,5-a]pyridin-2-amine;
(1*S, 2*S)—N-[5-[2-methyl-4-[((R)-1-methylazetidin-2-yl)methoxy]pyrazol-3-yl]pyrazolo[1,5-a]pyridin-2-yl]-2-phenyl-cyclopropanecarboxamide;
(1*R,2*R)—N-[5-[2-methyl-4-[((R)1-methylazetidin-2-yl)methoxy]pyrazol-3-yl]pyrazolo[1,5-a]pyridin-2-yl]-2-phenyl-cyclopropanecarboxamide;
(1S,2R)—N-[5-[2-methyl-4-[[(2R)-1-methylazetidin-2-yl]methoxy]pyrazol-3-yl]pyrazolo[1,5-a]pyridin-2-yl]-2-phenyl-cyclopropanecarboxamide;
(1*S,2*R)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide;
5-[4-[[(2R)-1-ethylazetidin-2-yl]methoxy]-2-methyl-pyrazol-3-yl]-N-[5-(trifluoromethyl)pyrazin-2-yl]pyrazolo[1,5-a]pyridin-2-amine;
5-[4-[[(2R)-1-ethylazetidin-2-yl]methoxy]-2-methyl-pyrazol-3-yl]-N-[2-(trifluoromethyl)pyrimidin-4-yl]pyrazolo[1,5-a]pyridin-2-amine;
5-[4-[[(2R)-1-ethylazetidin-2-yl]methoxy]-2-methyl-pyrazol-3-yl]-N-[2-methyl-6-(trifluoromethyl)pyrimidin-4-yl]pyrazolo[1,5-a]pyridin-2-amine;
5-[4-[[(2R)-1-ethylazetidin-2-yl]methoxy]-2-methyl-pyrazol-3-yl]-N-(2-methoxypyrimidin-4-yl)pyrazolo[1,5-a]pyridin-2-amine;
5-[4-[[(2R)-1-ethylazetidin-2-yl]methoxy]-2-methyl-pyrazol-3-yl]-N-[6-(trifluoromethyl)pyrimidin-4-yl]pyrazolo[1,5-a]pyridin-2-amine;
N-(4,5-dimethylpyrimidin-2-yl)-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(5-methoxy-2-pyridyl)pyrazolo[1,5-a]pyridin-2-amine;
5-[4-[[(2R)-1-ethylazetidin-2-yl]methoxy]-2-methyl-pyrazol-3-yl]-N-(6-methoxypyrimidin-4-yl) pyrazolo[1,5-a]pyridin-2-amine;
5-[4-[[(2S)-1-ethylazetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
5-[4-[[(2S)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine;
6-[[5-[4-[[(2R)-1-ethylazetidin-2-yl]methoxy]-2-methyl-pyrazol-3-yl]pyrazolo[1,5-a]pyridin-2-yl]amino]-2-methyl-pyridazin-3-one;
N-(2-cyclopropyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(6-ethyl-2-methyl-pyrimidin-4-yl)pyrazolo[1,5-a]pyridin-2-amine;
5-[4-[[(2R)-1-ethylazetidin-2-yl]methoxy]-2-methyl-pyrazol-3-yl]-N-(2-methoxy-6-methyl-pyrimidin-4-yl)pyrazolo[1,5-a]pyridin-2-amine;
N-[5-[4-[[(2R)-1-ethylazetidin-2-yl]methoxy]-2-methyl-pyrazol-3-yl]pyrazolo[1,5-a]pyridin-2-yl]-2-methyl-imidazo[1,2-b]pyridazin-6-amine;
N-[5-[4-[[(2R)-1-ethylazetidin-2-yl]methoxy]-2-methyl-pyrazol-3-yl]pyrazolo[1,5-a]pyridin-2-yl]imidazo[1,2-b]pyridazin-6-amine;
(R)-5-(4-((1-ethylazetidin-2-yl)methoxy)-1-methyl-1H-pyrazol-5-yl)-N-(5-fluoropyrimidin-2-yl) pyrazolo[1,5-a]pyridin-2-amine;
N-(2-tert-butyl-6-methyl-pyrimidin-4-yl)-5-[4-[[(2R)-1-ethylazetidin-2-yl]methoxy]-2-methyl-pyrazol-3-yl]pyrazolo[1,5-a]pyridin-2-amine;
6-[[5-[4-[[(2R)-1-ethylazetidin-2-yl]methoxy]-2-methyl-pyrazol-3-yl]pyrazolo[1,5-a]pyridin-2-yl]amino]-2,4-dimethyl-pyridazin-3-one;
N-[2-(cyclopropylmethyl)-6-methyl-pyrimidin-4-yl]-5-[4-[[(2R)-1-ethylazetidin-2-yl]methoxy]-2-methyl-pyrazol-3-yl]pyrazolo[1,5-a]pyridin-2-amine;
5-[4-[[(2R)-1-ethylazetidin-2-yl]methoxy]-2-methyl-pyrazol-3-yl]-N-(5-methoxypyrazin-2-yl)pyrazolo[1,5-a]pyridin-2-amine;
N-[5-(difluoromethoxy)pyrazin-2-yl]-5-[4-[[(2R)-1-ethylazetidin-2-yl]methoxy]-2-methyl-pyrazol-3-yl]pyrazolo[1,5-a]pyridin-2-amine;
N-[5-[2-methyl-4-[[(3R)-pyrrolidin-3-yl]oxymethyl]pyrazol-3-yl]pyrazolo[1,5-a]pyridin-2-yl]cyclopropanecarboxamide;
N-[5-[2-methyl-4-[[(1S,5R)-3-oxa-9-azabicyclo[3.3.1]nonan-7-yl]oxymethyl]pyrazol-3-yl]pyrazolo[1,5-a]pyridin-2-yl]cyclopropanecarboxamide;
(RS)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[2-methyl-4-[[(1S,5R)-9-methyl-3-oxa-9-azabicyclo[3.3.1]nonan-7-yl]oxymethyl]pyrazol-3-yl]pyrazolo[1,5-a]pyridin-2-yl]cyclopropanecarboxamide;
N-[5-[2-methyl-4-[[3-(trifluoromethyl)azetidin-3-yl]oxymethyl]pyrazol-3-yl]pyrazolo[1,5-a]pyridin-2-yl]cyclopropanecarboxamide;
N-[5-[2-methyl-4-[[(2S,3R)-2-methylazetidin-3-yl]oxymethyl]pyrazol-3-yl]pyrazolo[1,5-a]pyridin-2-yl]cyclopropanecarboxamide;
N-[5-[4-[[(3R,4S)-4-fluoropyrrolidin-3-yl]oxymethyl]-2-methyl-pyrazol-3-yl]pyrazolo[1,5-a]pyridin-2-yl]cyclopropanecarboxamide;
(*R)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[4-[[(2S,3R)-1,2-dimethylazetidin-3-yl]oxymethyl]-2-methyl-pyrazol-3-yl]pyrazolo[1,5-a]pyridin-2-yl]cyclopropanecarboxamide;
N-[5-[2-methyl-4-[(3-methylazetidin-3-yl)oxymethyl]pyrazol-3-yl]pyrazolo[1,5-a]pyridin-2-yl]cyclopropanecarboxamide;
N-[5-[4-[(1,3-dimethylazetidin-3-yl)oxymethyl]-2-methyl-pyrazol-3-yl]pyrazolo[1,5-a]pyridin-2-yl]cyclopropanecarboxamide;
N-[5-[4-[(3-ethylazetidin-3-yl)oxymethyl]-2-methyl-pyrazol-3-yl]pyrazolo[1,5-a]pyridin-2-yl]cyclopropanecarboxamide;
(3*S,4*S)—N-[5-[2-methyl-4-[(4-methylpyrrolidin-3-yl)oxymethyl]pyrazol-3-yl]pyrazolo[1,5-a]pyridin-2-yl]cyclopropanecarboxamide;
(3*S,4*S)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide;
(3*R,4*R)—N-[5-[2-methyl-4-[(4-methylpyrrolidin-3-yl)oxymethyl]pyrazol-3-yl]pyrazolo[1,5-a]pyridin-2-yl]cyclopropanecarboxamide;
(3*R, 4*R)—N-[5-[4-[(1,4-dimethylpyrrolidin-3-yl)oxymethyl]-2-methyl-pyrazol-3-yl]pyrazolo[1,5-a]pyridin-2-yl]cyclopropanecarboxamide;
3-[[4-[2-(cyclopropanecarbonylamino)pyrazolo[1,5-a]pyridin-5-yl]-5-methyl-isoxazol-3-yl]oxy methyl]azetidine-3-carboxamide;
N-[5-[5-methyl-3-[(3-phenylazetidin-3-yl)methoxy]isoxazol-4-yl]pyrazolo[1,5-a]pyridin-2-yl]cyclopropanecarboxamide;
(rac-cis)-N-[5-[3-(4-methoxypyrrolidin-3-yl)oxy-5-methyl-isoxazol-4-yl]pyrazolo[1,5-a]pyridin-2-yl]cyclopropanecarboxamide;
N-[5-[5-methyl-3-[[(1R,4R)-5-oxa-2-azabicyclo[2.2.1]heptan-4-yl]methoxy]isoxazol-4-yl]pyrazolo[1,5-a]pyridin-2-yl]cyclopropanecarboxamide;
N-(5-(3-(((3*S,4*R)-4-methoxypyrrolidin-3-yl)oxy)-5-methylisoxazol-4-yl)pyrazolo[1,5-a]pyridin-2-yl)cyclopropanecarboxamide;
N-[5-[5-methyl-3-[[(1S,4S)-2-methyl-5-oxa-2-azabicyclo[2.2.1]heptan-4-yl]methoxy]isoxazol-4-yl]pyrazolo[1,5-a]pyridin-2-yl]cyclopropanecarboxamide;
N-[5-[3-[(2R)-2-amino-2-phenyl-ethoxy]-5-methyl-isoxazol-4-yl]pyrazolo[1,5-a]pyridin-2-yl]cyclopropanecarboxamide;
(RS)—N-[5-[3-[(3-methoxypyrrolidin-3-yl)methoxy]-5-methyl-isoxazol-4-yl]pyrazolo[1,5-a]pyridin-2-yl]cyclopropanecarboxamide;
N-[5-[3-[(4-methoxy-4-piperidyl)methoxy]-5-methyl-isoxazol-4-yl]pyrazolo[1,5-a]pyridin-2-yl]cyclopropanecarboxamide;
N-[5-[3-[(2S)-2-(dimethylamino)-2-phenyl-ethoxy]-5-methyl-isoxazol-4-yl]pyrazolo[1,5-a]pyridin-2-yl]cyclopropanecarboxamide;
(rac-cis)-N-[5-[3-(4-fluoropyrrolidin-3-yl)oxy-5-methyl-isoxazol-4-yl]pyrazolo[1,5-a]pyridin-2-yl]cyclopropanecarboxamide;
(*S)—N-(5-(3-((3-methoxypyrrolidin-3-yl)methoxy)-5-methylisoxazol-4-yl)pyrazolo[1,5-a]pyridin-2-yl)cyclopropanecarboxamide;
N-(5-(3-(((3*S,4*R)-4-fluoropyrrolidin-3-yl)oxy)-5-methylisoxazol-4-yl)pyrazolo[1,5-a]pyridin-2-yl)cyclopropanecarboxamide;
(rac-cis)-N-[5-[3-[(3-fluoro-4-piperidyl)oxy]-5-methyl-isoxazol-4-yl]pyrazolo[1,5-a]pyridin-2-yl]cyclopropanecarboxamide;
Exo-Rac-N-[5-[3-(2-azabicyclo[2.2.1]heptan-5-yloxy)-5-methyl-isoxazol-4-yl]pyrazolo[1,5-a]pyridin-2-yl]cyclopropanecarboxamide;
N-[5-[3-[[(2R)-1-(cyanomethyl)azetidin-2-yl]methoxy]-5-methyl-isoxazol-4-yl]pyrazolo[1,5-a]pyridin-2-yl]cyclopropanecarboxamide;
N-[5-[5-methyl-3-[[(2R)-4,6,6-trimethylmorpholin-2-yl]methoxy]isoxazol-4-yl]pyrazolo[1,5-a]pyridin-2-yl]cyclopropanecarboxamide;
N-(5-(3-(((3*S,4*R)-3-fluoropiperidin-4-yl)oxy)-5-methylisoxazol-4-yl)pyrazolo[1,5-a]pyridin-2-yl)cyclopropanecarboxamide;
N-(5-(3-(((3*R,4*S)-3-fluoropiperidin-4-yl)oxy)-5-methylisoxazol-4-yl)pyrazolo[1,5-a]pyridin-2-yl)cyclopropanecarboxamide;
N-(5-(3-(((1*S,4*S,5*R)-2-azabicyclo[2.2.1]heptan-5-yl)oxy)-5-methylisoxazol-4-yl)pyrazolo[1,5-a]pyridin-2-yl)cyclopropanecarboxamide;
(*R)—N-[5-[3-(9-azaspiro[3.5]nonan-6-yloxy)-5-methyl-isoxazol-4-yl]pyrazolo[1,5-a]pyridin-2-yl]cyclopropanecarboxamide;
(*R)—N-[5-[5-methyl-3-[(9-methyl-9-azaspiro[3.5]nonan-6-yl)oxy]isoxazol-4-yl]pyrazolo[1,5-a]pyridin-2-yl]cyclopropanecarboxamide;
N-[5-[3-[(2S)-2-amino-2-(2-fluorophenyl)ethoxy]-5-methyl-isoxazol-4-yl]pyrazolo[1,5-a]pyridin-2-yl]cyclopropanecarboxamide;
N-[5-[3-[(2R)-2-amino-2-(2-fluorophenyl)ethoxy]-5-methyl-isoxazol-4-yl]pyrazolo[1,5-a]pyridin-2-yl]cyclopropanecarboxamide;
N-[5-[3-[(2R)-2-(dimethylamino)-2-(2-fluorophenyl)ethoxy]-5-methyl-isoxazol-4-yl]pyrazolo[1,5-a]pyridin-2-yl]cyclopropanecarboxamide;
N-[5-[3-[(2S)-2-amino-2-(2-pyridyl)ethoxy]-5-methyl-isoxazol-4-yl]pyrazolo[1,5-a]pyridin-2-yl]cyclopropanecarboxamide;
N-[5-[5-methyl-3-[(2S)-2-(methylamino)-2-(2-pyridyl)ethoxy]isoxazol-4-yl]pyrazolo[1,5-a]pyridin-2-yl]cyclopropanecarboxamide;
N-[5-[3-[(2S)-2-amino-2-phenyl-propoxy]-5-methyl-isoxazol-4-yl]pyrazolo[1,5-a]pyridin-2-yl]cyclopropanecarboxamide;
N-[5-[3-[(2S)-2-(dimethylamino)-2-phenyl-propoxy]-5-methyl-isoxazol-4-yl]pyrazolo[1,5-a]pyridin-2-yl]cyclopropanecarboxamide;
(3R, 3aS, 7aR)—N-[5-[3-(1,2,3,3a,5,6,7,7a-octahydropyrano[3,2-b]pyrrol-3-yloxy)-5-methyl-isoxazol-4-yl]pyrazolo[1,5-a]pyridin-2-yl]cyclopropanecarboxamide;
N-(5-(5-methyl-3-(((3*S,3a*R,7a*S)-octahydropyrano[3,2-b]pyrrol-3-yl)oxy)isoxazol-4-yl)pyrazolo[1,5-a]pyridin-2-yl)cyclopropanecarboxamide;
N-[5-[2-methyl-4-[[(3R)-pyrrolidin-3-yl]methoxy]pyrazol-3-yl]pyrazolo[1,5-a]pyridin-2-yl]cyclopropanecarboxamide;
N-[5-[2-methyl-4-[[(2S)-morpholin-2-yl]methoxy]pyrazol-3-yl]pyrazolo[1,5-a]pyridin-2-yl]cyclopropanecarboxamide;
N-[5-[4-[(1-aminocyclobutyl)methoxy]-2-methyl-pyrazol-3-yl]pyrazolo[1,5-a]pyridin-2-yl]cyclopropanecarboxamide;
(rac-cis)-N-[5-[4-(4-methoxypyrrolidin-3-yl)oxy-2-methyl-pyrazol-3-yl]pyrazolo[1,5-a]pyridin-2-yl]cyclopropanecarboxamide;
N-[5-[2-methyl-4-[(3S,5R)-5-methylpyrrolidin-3-yl]oxy-pyrazol-3-yl]pyrazolo[1,5-a]pyridin-2-yl]cyclopropanecarboxamide;
N-[5-[2-methyl-4-[[(1R,4R)-5-oxa-2-azabicyclo[2.2.1]heptan-4-yl]methoxy]pyrazol-3-yl]pyrazolo[1,5-a]pyridin-2-yl]cyclopropanecarboxamide;
N-[5-[4-[(2S)-2-amino-2-phenyl-ethoxy]-2-methyl-pyrazol-3-yl]pyrazolo[1,5-a]pyridin-2-yl]cyclopropanecarboxamide;
N-[5-[2-methyl-4-[(3S)-pyrrolidin-3-yl]oxy-pyrazol-3-yl]pyrazolo[1,5-a]pyridin-2-yl]cyclopropanecarboxamide;
N-[5-[4-[(2R)-2-amino-2-phenyl-ethoxy]-2-methyl-pyrazol-3-yl]pyrazolo[1,5-a]pyridin-2-yl]cyclopropanecarboxamide;
(3*R, 4*R)—N-[5-[2-methyl-4-(4-methylpyrrolidin-3-yl)oxy-pyrazol-3-yl]pyrazolo[1,5-a]pyridin-2-yl]cyclopropanecarboxamide;
N-[5-[2-methyl-4-[[(2R)-1-methylpyrrolidin-2-yl]methoxy]pyrazol-3-yl]pyrazolo[1,5-a]pyridin-2-yl]cyclopropanecarboxamide;
(RS)—N-[5-[4-(5-azaspiro[2.3]hexan-6-ylmethoxy)-2-methyl-pyrazol-3-yl]pyrazolo[1,5-a]pyridin-2-yl]cyclopropanecarboxamide;
N-[5-[4-[[(2R)-1-(2-fluoroethyl)azetidin-2-yl]methoxy]-2-methyl-pyrazol-3-yl]pyrazolo[1,5-a]pyridin-2-yl]cyclopropanecarboxamide;
N-[5-[4-[(3R,4S)-4-fluoropyrrolidin-3-yl]oxy-2-methyl-pyrazol-3-yl]pyrazolo[1,5-a]pyridin-2-yl]cyclopropanecarboxamide;
N-[5-[4-[[(2R)-1-(2-methoxyethyl)azetidin-2-yl]methoxy]-2-methyl-pyrazol-3-yl]pyrazolo[1,5-a]pyridin-2-yl]cyclopropanecarboxamide;
N-[5-[4-[[(2R)-1-isobutylazetidin-2-yl]methoxy]-2-methyl-pyrazol-3-yl]pyrazolo[1,5-a]pyridin-2-yl]cyclopropanecarboxamide;
N-[5-[4-[[(2R)-1-(2-hydroxy-2-methyl-propyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-propylazetidin-2-yl]methoxy]pyrazol-3-yl]pyrazolo[1,5-a]pyridin-2-yl]cyclopropanecarboxamide;
N-[5-[4-[[(2R)-1-(cyclopropylmethyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2S)-1-(trideuteriomethyl)azetidin-2-yl]methoxy]pyrazol-3-yl]pyrazolo[1,5-a]pyridin-2-yl]cyclopropanecarboxamide;
N-[5-[2-methyl-4-[[(2R)-1-(trideuteriomethyl)pyrrolidin-2-yl]methoxy]pyrazol-3-yl]pyrazolo[1,5-a]pyridin-2-yl]cyclopropanecarboxamide;
N-[5-[2-methyl-4-[[(2R)-1-(trideuteriomethyl)azetidin-2-yl]methoxy]pyrazol-3-yl]pyrazolo[1,5-a]pyridin-2-yl]cyclopropanecarboxamide;
N-[5-[4-[[(2R)-1-acetonylazetidin-2-yl]methoxy]-2-methyl-pyrazol-3-yl]pyrazolo[1,5-a]pyridin-2-yl]cyclopropanecarboxamide;
N-[5-[4-[[(2R)-1-[(2R)-2-hydroxypropyl]azetidin-2-yl]methoxy]-2-methyl-pyrazol-3-yl]pyrazolo[1,5-a]pyridin-2-yl]cyclopropanecarboxamide;
N-[5-[4-[[(2R)-1-[(2S)-2-hydroxypropyl]azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-(1,1,2,2,2-pentadeuterioethyl)azetidin-2-yl]methoxy]pyrazol-3-yl]pyrazolo[1,5-a]pyridin-2-yl]cyclopropanecarboxamide;
N-[5-[4-[[(2R)-1-(2-cyano-2-methyl-propyl)azetidin-2-yl]methoxy]-2-methyl-pyrazol-3-yl]pyrazolo[1,5-a]pyridin-2-yl]cyclopropanecarboxamide;
N-[5-[4-[[(2R,5R)-1,5-dimethylpyrrolidin-2-yl]methoxy]-2-methyl-pyrazol-3-yl]pyrazolo[1,5-a]pyridin-2-yl]cyclopropanecarboxamide;
N-[5-[2-methyl-4-[[(2R,4S)-1-methyl-4-phenyl-pyrrolidin-2-yl]methoxy]pyrazol-3-yl]pyrazolo[1,5-a]pyridin-2-yl]cyclopropanecarboxamide;
N-[5-[2-methyl-4-[[(2R,4R)-1-methyl-4-phenyl-pyrrolidin-2-yl]methoxy]pyrazol-3-yl]pyrazolo[1,5-a]pyridin-2-yl]cyclopropanecarboxamide;
N-(5-(1-methyl-4-(((R)-1-(((R)-tetrahydrofuran-2-yl)methyl)azetidin-2-yl)methoxy)-1H-pyrazol-5-yl)pyrazolo[1,5-a]pyridin-2-yl)cyclopropanecarboxamide;
5-[4-[[(2R)-azetidin-2-yl]methoxy]-2-(difluoromethyl)pyrazol-3-yl]-N-(2,6-dimethylpyrimidin-4-yl)pyrazolo[1,5-a]pyridin-2-amine;
(*R)-5-(1-(difluoromethyl)-4-((3-methoxypyrrolidin-3-yl)methoxy)-1H-pyrazol-5-yl)-N-(2,6-dimethylpyrimidin-4-yl)pyrazolo[1,5-a]pyridin-2-amine;
(*R)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine;
(*S)-5-(1-(difluoromethyl)-4-((3-fluoropyrrolidin-3-yl)methoxy)-1H-pyrazol-5-yl)-N-(2,6-dimethylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(5-methylpyrazin-2-yl)pyrazolo[1,5-a]pyridin-2-amine;
5-[2-methyl-4-[(3S)-1-methylpyrrolidin-3-yl]oxy-pyrazol-3-yl]-N-(6-methylpyrimidin-4-yl)pyrazolo[1,5-a]pyridin-2-amine;
5-[2-methyl-4-[(3S)-1-methylpyrrolidin-3-yl]oxy-pyrazol-3-yl]-N-[6-(trifluoromethyl)-2-pyridyl]pyrazolo[1,5-a]pyridin-2-amine;
N-(2,6-dimethylpyrimidin-4-yl)-5-[2-methyl-4-[(3S)-1-methylpyrrolidin-3-yl]oxy-pyrazol-3-yl]pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]pyrazolo[1,5-a]pyrimidin-5-amine;
N-(6,7-dihydro-5H-cyclopenta[c]pyridazin-3-yl)-5-[2-methyl-4-[(3S)-1-methylpyrrolidin-3-yl]oxy-pyrazol-3-yl]pyrazolo[1,5-a]pyridin-2-amine;
N-(2-isopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(6-isopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(5,6-dimethylpyridazin-3-yl)-5-[2-methyl-4-[(3S)-1-methylpyrrolidin-3-yl]oxy-pyrazol-3-yl]pyrazolo[1,5-a]pyridin-2-amine;
5-[4-[[(2R)-azetidin-2-yl]methoxy]-2-methyl-pyrazol-3-yl]-N-[6-methyl-2-(trifluoromethyl)pyrimidin-4-yl]pyrazolo[1,5-a]pyridin-2-amine;
N-(2,6-dimethylpyrimidin-4-yl)-5-[4-[[(2R)-1-(2-fluoroethyl)azetidin-2-yl]methoxy]-2-methyl-pyrazol-3-yl]pyrazolo[1,5-a]pyridin-2-amine;
5-[4-[[(2R)-azetidin-2-yl]methoxy]-2-methyl-pyrazol-3-yl]-N-(5,6-dimethylpyridazin-3-yl)pyrazolo[1,5-a]pyridin-2-amine;
N-(5,6-dimethylpyridazin-3-yl)-5-[4-[[(2R)-1-(2-fluoroethyl)azetidin-2-yl]methoxy]-2-methyl-pyrazol-3-yl]pyrazolo[1,5-a]pyridin-2-amine;
5-[4-[[(2R)-1-(2-fluoroethyl)azetidin-2-yl]methoxy]-2-methyl-pyrazol-3-yl]-N-(6-methylpyrimidin-4-yl)pyrazolo[1,5-a]pyridin-2-amine;
5-[4-[[(2R)-1-(2-fluoroethyl)azetidin-2-yl]methoxy]-2-methyl-pyrazol-3-yl]-N-(6-methylpyrazin-2-yl)pyrazolo[1,5-a]pyridin-2-amine;
5-[4-[[(2R)-1-(2-fluoroethyl)azetidin-2-yl]methoxy]-2-methyl-pyrazol-3-yl]-N-(5-methylpyrazin-2-yl)pyrazolo[1,5-a]pyridin-2-amine;
N-[5-[2-methyl-4-[(3S)-1-methylpyrrolidin-3-yl]oxy-pyrazol-3-yl]pyrazolo[1,5-a]pyridin-2-yl]-7,8-dihydro-5H-pyrano[4,3-b]pyridin-2-amine;
N-(2-cyclopropylpyrimidin-4-yl)-5-[2-methyl-4-[(3S)-1-methylpyrrolidin-3-yl]oxy-pyrazol-3-yl]pyrazolo[1,5-a]pyridin-2-amine;
N-(2,6-dimethylpyrimidin-4-yl)-5-[2-methyl-4-[[(2R)-1-methylpyrrolidin-2-yl]methoxy]pyrazol-3-yl]pyrazolo[1,5-a]pyridin-2-amine;
5-[2-methyl-4-[[(2R)-1-methylpyrrolidin-2-yl]methoxy]pyrazol-3-yl]-N-[6-methyl-2-(trifluoromethyl)pyrimidin-4-yl]pyrazolo[1,5-a]pyridin-2-amine;
N-(6-cyclopropylpyridazin-3-yl)-5-[2-methyl-4-[[(2R)-1-methylpyrrolidin-2-yl]methoxy]pyrazol-3-yl]pyrazolo[1,5-a]pyridin-2-amine;
5-[2-methyl-4-[[(2R)-1-methylpyrrolidin-2-yl]methoxy]pyrazol-3-yl]-N-(6-methylpyrimidin-4-yl)pyrazolo[1,5-a]pyridin-2-amine;
5-[2-methyl-4-[[(2R)-1-methylpyrrolidin-2-yl]methoxy]pyrazol-3-yl]-N-(2-methylpyrimidin-4-yl)pyrazolo[1,5-a]pyridin-2-amine;
5-[2-methyl-4-[[(2R)-1-methylpyrrolidin-2-yl]methoxy]pyrazol-3-yl]-N-(6-methylpyridazin-3-yl)pyrazolo[1,5-a]pyridin-2-amine;
5-[4-(3-azabicyclo[2.1.1]hexan-4-ylmethoxy)-2-methyl-pyrazol-3-yl]-N-(2,6-dimethylpyrimidin-4-yl)pyrazolo[1,5-a]pyridin-2-amine;
N-(2,6-dimethylpyrimidin-4-yl)-5-[4-[[(2R)-1-ethylpyrrolidin-2-yl]methoxy]-2-methyl-pyrazol-3-yl]pyrazolo[1,5-a]pyridin-2-amine;
N-(2,6-dimethylpyrimidin-4-yl)-5-[2-methyl-4-[[(2R)-1-(trideuteriomethyl)azetidin-2-yl]methoxy]pyrazol-3-yl]pyrazolo[1,5-a]pyridin-2-amine;
5-[4-[[(2R)-azetidin-2-yl]methoxy]-2-methyl-pyrazol-3-yl]-N-(6-pyrazol-1-ylpyrazin-2-yl)pyrazolo[1,5-a]pyridin-2-amine;
(2S)-1-[(2R)-2-[[5-[2-[(2,6-dimethylpyrimidin-4-yl)amino]pyrazolo[1,5-a]pyridin-5-yl]-1-methy 1-pyrazol-4-yl]oxymethyl]azetidin-1-yl]propan-2-ol;
N-(2,6-dimethylpyrimidin-4-yl)-5-[2-methyl-4-[[(2R)-1-(1,1,2,2,2-pentadeuterioethyl)pyrrolidin-2-yl]methoxy]pyrazol-3-yl]pyrazolo[1,5-a]pyridin-2-amine;
N-(2,6-dimethylpyrimidin-4-yl)-5-[2-methyl-4-[[(2R)-1-(1,1,2,2,2-pentadeuterioethyl)azetidin-2-yl]methoxy]pyrazol-3-yl]pyrazolo[1,5-a]pyridin-2-amine;
(2*R, 3*S)—N-(2,6-dimethylpyrimidin-4-yl)-5-[2-methyl-4-[(2-methyltetrahydrofuran-3-yl)methoxy]pyrazol-3-yl]pyrazolo[1,5-a]pyridin-2-amine;
(2S)-1-[(2R)-2-[[1-methyl-5-[2-[(5-methylpyrazin-2-yl)amino]pyrazolo[1,5-a]pyridin-5-yl]pyrazol-4-yl]oxymethyl]azetidin-1-yl]propan-2-ol;
(2S)-1-[(2R)-2-[[5-[2-[(5,6-dimethylpyrazin-2-yl)amino]pyrazolo[1,5-a]pyridin-5-yl]-1-methyl-pyrazol-4-yl]oxymethyl]azetidin-1-yl]propan-2-ol;
N-(2,6-dimethylpyrimidin-4-yl)-5-[2-methyl-4-[(2S,3R)-2-methylpyrrolidin-3-yl]oxy-pyrazol-3-yl]pyrazolo[1,5-a]pyridin-2-amine;
(*S)—N-(2,6-dimethylpyrimidin-4-yl)-5-[4-(6,9-dioxaspiro[4.4]nonan-8-ylmethoxy)-2-methyl-pyrazol-3-yl]pyrazolo[1,5-a]pyridin-2-amine;
(2R,3S)-2-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol;
(1S)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol;
(S)-1-cyclobutyl-2-[5-[2-[(2,6-dimethylpyrimidin-4-yl)amino]pyrazolo[1,5-a]pyridin-5-yl]-1-methyl-pyrazol-4-yl]oxy-ethanol;
(2S)-1-[(2R)-2-[[1-methyl-5-[2-[[2-methyl-6-(trifluoromethyl)pyrimidin-4-yl]amino]pyrazolo[1,5-a]pyridin-5-yl]pyrazol-4-yl]oxymethyl]azetidin-1-yl]propan-2-ol;
(*S)-2-cyclopropyl-1-[5-[2-[(2,6-dimethylpyrimidin-4-yl)amino]pyrazolo[1,5-a]pyridin-5-yl]-1-methyl-pyrazol-4-yl]oxy-propan-2-ol;
N-(2,6-dimethylpyrimidin-4-yl)-5-[4-[[(2R,4S)-1,4-dimethylpyrrolidin-2-yl]methoxy]-2-methyl-pyrazol-3-yl]pyrazolo[1,5-a]pyridin-2-amine;
(*S)-1-(3-bicyclo[1.1.1]pentanyl)-2-[5-[2-[(2,6-dimethylpyrimidin-4-yl)amino]pyrazolo[1,5-a]pyridin-5-yl]-1-methyl-pyrazol-4-yl]oxy-ethanol;
(*R)—N-(2,6-dimethylpyrimidin-4-yl)-5-[4-[(5,5-dimethyltetrahydrofuran-3-yl)methoxy]-2-methyl-pyrazol-3-yl]pyrazolo[1,5-a]pyridin-2-amine;
5-[2-methyl-4-[[(2S)-1-methylazetidin-2-yl]methoxy]pyrazol-3-yl]-N-[6-methyl-2-(trifluoromethyl)pyrimidin-4-yl]pyrazolo[1,5-a]pyridin-2-amine;
5-[4-[2-amino-2-(1-methylcyclopropyl)ethoxy]-2-methyl-pyrazol-3-yl]-N-(2,6-dimethylpyrimidin-4-yl)pyrazolo[1,5-a]pyridin-2-amine;
5-[4-[2-amino-2-(3-bicyclo[1.1.1]pentanyl)ethoxy]-2-methyl-pyrazol-3-yl]-N-(2,6-dimethylpyrimidin-4-yl)pyrazolo[1,5-a]pyridin-2-amine;
methyl 5-[[5-[4-[[(2R)-1-ethylazetidin-2-yl]methoxy]-2-methyl-pyrazol-3-yl]pyrazolo[1,5-a]pyridin-2-yl]amino]pyrazine-2-carboxylate;
N-[5-[2-ethyl-4-[[(3R)-pyrrolidin-3-yl]methoxy]pyrazol-3-yl]pyrazolo[1,5-a]pyridin-2-yl]cyclopropanecarboxamide;
N-[5-[2-methyl-4-[[(1R,5S)-3-oxa-9-azabicyclo[3.3.1]nonan-7-yl]oxy]pyrazol-3-yl]pyrazolo[1,5-a]pyridin-2-yl]cyclopropanecarboxamide;
N-[5-[4-[(1,3-dimethylazetidin-3-yl)methoxy]-2-methyl-pyrazol-3-yl]pyrazolo[1,5-a]pyridin-2-yl]cyclopropanecarboxamide;
N-[5-[2-methyl-4-[[(2R)-1-(2,2,2-trifluoroethyl)azetidin-2-yl]methoxy]pyrazol-3-yl]pyrazolo[1,5-a]pyridin-2-yl]cyclopropanecarboxamide;
N-[5-[5-methyl-3-(4-piperidyloxy)isoxazol-4-yl]pyrazolo[1,5-a]pyridin-2-yl]cyclopropanecarboxamide;
*S, *R—N-[5-[3-(4-methoxy-1-methyl-pyrrolidin-3-yl)oxy-5-methyl-isoxazol-4-yl]pyrazolo[1,5-a]pyridin-2-yl]cyclopropanecarboxamide;
5-[3-[(3-methoxyazetidin-3-yl)methoxy]-5-methyl-isoxazol-4-yl]-N-pyrazin-2-yl-pyrazolo[1,5-a]pyridin-2-amine;
N-[5-[3-[(3-aminocyclobutyl)methoxy]-5-methyl-isoxazol-4-yl]pyrazolo[1,5-a]pyridin-2-yl]cyclopropanecarboxamide;
5-[3-[[(2R)-azetidin-2-yl]methoxy]-5-methyl-isoxazol-4-yl]-N-(2,6-dimethylpyrimidin-4-yl)pyrazolo[1,5-a]pyridin-2-amine;
N-[5-[5-methyl-3-[[(2S)-1,3,3-trimethylazetidin-2-yl]methoxy]isoxazol-4-yl]pyrazolo[1,5-a]pyridin-2-yl]cyclopropanecarboxamide;
5-[5-methyl-3-[(3S,5R)-5-methylpyrrolidin-3-yl]oxy-isoxazol-4-yl]-N-(5-methylpyrazin-2-yl)pyrazolo[1,5-a]pyridin-2-amine;
5-[5-methyl-3-[[(2R)-1-methylazetidin-2-yl]methoxy]isoxazol-4-yl]-N-(5-methylpyrazin-2-yl)pyrazolo[1,5-a]pyridin-2-amine;
N-[5-[3-[(2S)-2-(dimethylamino)-2-(2-fluorophenyl)ethoxy]-5-methyl-isoxazol-4-yl]pyrazolo[1,5-a]pyridin-2-yl]cyclopropanecarboxamide;
N-[5-[3-[(2S)-2-(dimethylamino)-2-(2-pyridyl)ethoxy]-5-methyl-isoxazol-4-yl]pyrazolo[1,5-a]pyridin-2-yl]cyclopropanecarboxamide;
N-[5-[3-[(2R)-2-amino-2-phenyl-propoxy]-5-methyl-isoxazol-4-yl]pyrazolo[1,5-a]pyridin-2-yl]cyclopropanecarboxamide;
5-[3-[(3S,5S)-5-(difluoromethyl)pyrrolidin-3-yl]oxy-5-methyl-isoxazol-4-yl]-N-(2,6-dimethylpyrimidin-4-yl)pyrazolo[1,5-a]pyridin-2-amine;
N-[5-[3-[(2S)-2-amino-2-cyclopropyl-ethoxy]-5-methyl-isoxazol-4-yl]pyrazolo[1,5-a]pyridin-2-yl]cyclopropanecarboxamide;
N-[6-[5-methyl-3-[[(2R)-1-methylazetidin-2-yl]methoxy]isoxazol-4-yl]imidazo[1,2-a]pyridin-2-yl]cyclopropanecarboxamide;
N-[5-[5-methyl-3-[(3S)-pyrrolidin-3-yl]oxy-isothiazol-4-yl]pyrazolo[1,5-a]pyridin-2-yl]cyclopropanecarboxamide;
N-[5-[5-methyl-3-[[(2R)-morpholin-2-yl]methoxy]isothiazol-4-yl]pyrazolo[1,5-a]pyridin-2-yl]cyclopropanecarboxamide;
N-[5-[5-methyl-3-[[(2R)-4-methylmorpholin-2-yl]methoxy]isothiazol-4-yl]pyrazolo[1,5-a]pyridin-2-yl]cyclopropanecarboxamide;
N-[5-[2-methyl-4-[[(2R)-4-methylmorpholin-2-yl]methoxy]pyrazol-3-yl]pyrazolo[1,5-a]pyridin-2-yl]cyclopropanecarboxamide;
N-[5-[4-[(3-aminocyclobutyl)methoxy]-2-methyl-pyrazol-3-yl]pyrazolo[1,5-a]pyridin-2-yl]cyclopropanecarboxamide;
N-[5-[2-methyl-4-[(3R)-pyrrolidin-3-yl]oxy-pyrazol-3-yl]pyrazolo[1,5-a]pyridin-2-yl]cyclopropanecarboxamide;
N-[5-[4-[(3S,5R)-1,5-dimethylpyrrolidin-3-yl]oxy-2-methyl-pyrazol-3-yl]pyrazolo[1,5-a]pyridin-2-yl]cyclopropanecarboxamide;
(2*S, 3*R)—N-[5-[4-[(1,2-dimethylazetidin-3-yl)methoxy]-2-methyl-pyrazol-3-yl]pyrazolo[1,5-a]pyridin-2-yl]cyclopropanecarboxamide;
N-[5-[2-methyl-4-[(3S,5S)-5-methylpyrrolidin-3-yl]oxy-pyrazol-3-yl]pyrazolo[1,5-a]pyridin-2-yl]cyclopropanecarboxamide;
5-[2-methyl-4-[(3S)-1-methylpyrrolidin-3-yl]oxy-pyrazol-3-yl]-N-[5-(trifluoromethyl)-2-pyridyl]pyrazolo[1,5-a]pyridin-2-amine;
5-[2-methyl-4-[(3S)-1-methylpyrrolidin-3-yl]oxy-pyrazol-3-yl]-N-[4-(trifluoromethyl)-2-pyridyl]pyrazolo[1,5-a]pyridin-2-amine;
N-[5-[2-methyl-4-[[(2S,3S)-3-methylazetidin-2-yl]methoxy]pyrazol-3-yl]pyrazolo[1,5-a]pyridin-2-yl]cyclopropanecarboxamide;
N-[5-[2-methyl-4-[(3S)-1-methylpyrrolidin-3-yl]oxy-pyrazol-3-yl]pyrazolo[1,5-a]pyridin-2-yl]cinnolin-3-amine;
N-[5-[2-methyl-4-[(3S)-1-methylpyrrolidin-3-yl]oxy-pyrazol-3-yl]pyrazolo[1,5-a]pyridin-2-yl]quinoxalin-2-amine;
5-[2-methyl-4-[(3S)-1-methylpyrrolidin-3-yl]oxy-pyrazol-3-yl]-N-pyrazolo[1,5-a]pyridin-2-yl-pyrazolo[1,5-a]pyridin-2-amine;
N-[5-[4-[[(3R,4R)-4-hydroxypyrrolidin-3-yl]methoxy]-2-methyl-pyrazol-3-yl]pyrazolo[1,5-a]pyridin-2-yl]cyclopropanecarboxamide;
(R)—N-(5-(4-(azetidin-2-ylmethoxy)-1-methyl-1H-pyrazol-5-yl)pyrazolo[1,5-a]pyridin-2-yl)cyclopropanecarboxamide;
N-[5-[4-[[(2R)-1-cyclopropylazetidin-2-yl]methoxy]-2-methyl-pyrazol-3-yl]pyrazolo[1,5-a]pyridin-2-yl]cyclopropanecarboxamide;
N-[5-[4-[(2S)-2-amino-3,3-dimethyl-butoxy]-2-methyl-pyrazol-3-yl]pyrazolo[1,5-a]pyridin-2-yl]cyclopropanecarboxamide;
N-(5,6-dimethylpyrazin-2-yl)-5-[2-methyl-4-[(3S)-1-methylpyrrolidin-3-yl]oxy-pyrazol-3-yl]pyrazolo[1,5-a]pyridin-2-amine;
N-[5-[4-[[(2R)-1-(2-fluoroethyl)pyrrolidin-2-yl]methoxy]-2-methyl-pyrazol-3-yl]pyrazolo[1,5-a]pyridin-2-yl]cyclopropanecarboxamide;
N-[5-[4-[[(3R,4R)-4-methoxypyrrolidin-3-yl]methoxy]-2-methyl-pyrazol-3-yl]pyrazolo[1,5-a]pyridin-2-yl]cyclopropanecarboxamide;
(1*S, 2*S, 4*R)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine;
(*S)—N-[5-[2-methyl-4-[[1-(tetrahydrofuran-2-ylmethyl)azetidin-2-yl]methoxy]pyrazol-3-yl]pyrazolo[1,5-a]pyridin-2-yl]cyclopropanecarboxamide;
N-(2,6-dimethylpyrimidin-4-yl)-5-[4-[(2S,3R)-1,2-dimethylpyrrolidin-3-yl]oxy-2-methyl-pyrazol-3-yl]pyrazolo[1,5-a]pyridin-2-amine;
N-(2,6-dimethylpyrimidin-4-yl)-5-[2-(trideuteriomethyl)-4-[[(2R)-1-(trideuteriomethyl)azetidin-2-yl]methoxy]pyrazol-3-yl]pyrazolo[1,5-a]pyridin-2-amine;
(1*R)-2-[5-[2-[(2,6-dimethylpyrimidin-4-yl)amino]pyrazolo[1,5-a]pyridin-5-yl]-1-methyl-pyrazol-4-yl]oxy-1-(1-methylcyclopropyl)ethanol;
*S-1-methylcyclopropyl-2-[5-[2-[(2,6-dimethylpyrimidin-4-yl)amino]pyrazolo[1,5-a]pyridin-5-yl]-1-methyl-pyrazol-4-yl]oxy-ethanol;
*S—N-(2,6-dimethylpyrimidin-4-yl)-5-[2-methyl-4-(7-oxaspiro[3.4]octan-6-ylmethoxy)pyrazol-3-yl]pyrazolo[1,5-a]pyridin-2-amine;
N-(5,6-dimethylpyrazin-2-yl)-5-[4-[[(2S)-1-ethylazetidin-2-yl]methoxy]-2-methyl-pyrazol-3-yl]pyrazolo[1,5-a]pyridin-2-amine;
N-[5-[4-(azetidin-3-yloxymethyl)-2-methyl-pyrazol-3-yl]pyrazolo[1,5-a]pyridin-2-yl]cyclopropanecarboxamide;
N-[5-[2-methyl-4-[(1-methylazetidin-3-yl)oxymethyl]pyrazol-3-yl]pyrazolo[1,5-a]pyridin-2-yl]cyclopropanecarboxamide;
N-[5-[2-methyl-4-[[(3R)-1-methylpyrrolidin-3-yl]oxymethyl]pyrazol-3-yl]pyrazolo[1,5-a]pyridin-2-yl]cyclopropanecarboxamide;
N-[5-[2-methyl-4-[[(3S)-pyrrolidin-3-yl]oxymethyl]pyrazol-3-yl]pyrazolo[1,5-a]pyridin-2-yl]cyclopropanecarboxamide;
Rac-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
Trans-*R, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
Trans-*S, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
(rac-cis)-N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS—*R, *R—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS—*S, *S—N-(2,6-dimethylpyrimidin-4-yl)-5-[4-[(1,4-dimethylpyrrolidin-3-yl)oxymethyl]-2-methyl-pyrazol-3-yl]pyrazolo[1,5-a]pyridin-2-amine;
CIS-Rac-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*R, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
Trans-*S, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
(rac-trans)-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*S, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Trans-*R, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
Rac-CIS-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
CIS—*R, *R-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
CIS—*R, *R-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
CIS—*S, *S-5-[4-[(1,4-dimethylpyrrolidin-3-yl)oxymethyl]-2-methyl-pyrazol-3-yl]-N-(6-methylpyrazin-2-yl)pyrazolo[1,5-a]pyridin-2-amine;
CIS—*S, *S-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
N-(5-tert-butylpyrazin-2-yl)-5-[2-methyl-4-[[(2R)-1-methylazetidin-2-yl]methoxy]pyrazol-3-yl]pyrazolo[1,5-a]pyridin-2-amine;
N-[5-[5-methyl-3-[(3S)-1-methylpyrrolidin-3-yl]oxy-isoxazol-4-yl]pyrazolo[1,5-a]pyridin-2-yl]cyclopropanecarboxamide;
N-[5-[5-methyl-3-[[(3R)-3-piperidyl]methoxy]isoxazol-4-yl]pyrazolo[1,5-a]pyridin-2-yl]cyclopropanecarboxamide;
N-[5-[5-methyl-3-[(3S)-1-(trideuteriomethyl)pyrrolidin-3-yl]oxy-isoxazol-4-yl]pyrazolo[1,5-a]pyridin-2-yl]cyclopropanecarboxamide;
(3*R,4*S)—N-[5-[3-(4-methoxypyrrolidin-3-yl)oxy-5-methyl-isoxazol-4-yl]pyrazolo[1,5-a]pyridin-2-yl]cyclopropanecarboxamide;
N-[5-[3-(3-aminocyclobutoxy)-5-methyl-isoxazol-4-yl]pyrazolo[1,5-a]pyridin-2-yl]cyclopropane carboxamide;
N-[5-[3-[(3-aminocyclobutyl)methoxy]-5-methyl-isoxazol-4-yl]pyrazolo[1,5-a]pyridin-2-yl]cyclopropanecarboxamide;
N-[5-[5-methyl-3-[[(2R)-1-methylpyrrolidin-2-yl]methoxy]isoxazol-4-yl]pyrazolo[1,5-a]pyridin-2-yl]cyclopropanecarboxamide;
N-[5-[3-[(3S,5S)-5-(difluoromethyl)pyrrolidin-3-yl]oxy-5-methyl-isoxazol-4-yl]pyrazolo[1,5-a]pyridin-2-yl]cyclopropanecarboxamide;
N-(5,6-dimethylpyridazin-3-yl)-5-[3-[(3S,5S)-5-(methoxymethyl)pyrrolidin-3-yl]oxy-5-methyl-isoxazol-4-yl]pyrazolo[1,5-a]pyridin-2-amine;
N-(5,6-dimethylpyridazin-3-yl)-5-[3-[(3S,5S)-5-(methoxymethyl)-1-methyl-pyrrolidin-3-yl]oxy-5-methyl-isoxazol-4-yl]pyrazolo[1,5-a]pyridin-2-amine;
5-[4-[[(2R)-azetidin-2-yl]methoxy]-2-methyl-pyrazol-3-yl]-N-(6-methylpyridazin-3-yl)pyrazolo[1,5-a]pyridin-2-amine;
5-[4-[[(2R)-azetidin-2-yl]methoxy]-2-methyl-pyrazol-3-yl]-N-(5-methylpyrazin-2-yl)pyrazolo[1,5-a]pyridin-2-amine;
5-[4-[[(2R)-azetidin-2-yl]methoxy]-2-methyl-pyrazol-3-yl]-N-(6-cyclopropylpyridazin-3-yl)pyrazolo[1,5-a]pyridin-2-amine;
N-(5-fluoropyrimidin-2-yl)-5-[2-methyl-4-[[(2R)-1-methylazetidin-2-yl]methoxy]pyrazol-3-yl]pyrazolo[1,5-a]pyridin-2-amine;
N-[5-[2-methyl-4-[[(3R)-1-methylpyrrolidin-3-yl]methoxy]pyrazol-3-yl]pyrazolo[1,5-a]pyridin-2-yl]cyclopropanecarboxamide;
N-[5-[4-(azetidin-3-yloxy)-2-methyl-pyrazol-3-yl]pyrazolo[1,5-a]pyridin-2-yl]cyclopropanecarboxamide;
N-[5-[2-ethyl-4-[[(3R)-1-methylpyrrolidin-3-yl]methoxy]pyrazol-3-yl]pyrazolo[1,5-a]pyridin-2-yl]cyclopropanecarboxamide;
N-[5-[2-methyl-4-(1-methylazetidin-3-yl)oxy-pyrazol-3-yl]pyrazolo[1,5-a]pyridin-2-yl]cyclopropanecarboxamide;
N-[5-[4-[(3-aminocyclobutyl)methoxy]-2-methyl-pyrazol-3-yl]pyrazolo[1,5-a]pyridin-2-yl]cyclopropanecarboxamide;
(*R)—N-[5-[2-methyl-4-(3,3,3-trifluoro-2-hydroxy-propoxy)pyrazol-3-yl]pyrazolo[1,5-a]pyridin-2-yl]cyclopropanecarboxamide;
N-[5-[4-[(3-aminooxetan-3-yl)methoxy]-2-methyl-pyrazol-3-yl]pyrazolo[1,5-a]pyridin-2-yl]cyclopropanecarboxamide;
N-[5-[4-[(2R)-2-hydroxy-2-phenyl-ethoxy]-2-methyl-pyrazol-3-yl]pyrazolo[1,5-a]pyridin-2-yl]cyclopropanecarboxamide;
N-[5-[4-(4-hydroxycyclohexoxy)-2-methyl-pyrazol-3-yl]pyrazolo[1,5-a]pyridin-2-yl]cyclopropanecarboxamide;
N-[5-[4-[(3-fluoro-1-methyl-azetidin-3-yl)methoxy]-2-methyl-pyrazol-3-yl]pyrazolo[1,5-a]pyridin-2-yl]cyclopropanecarboxamide;
N-[5-[4-[(3-methoxy-1-methyl-azetidin-3-yl)methoxy]-2-methyl-pyrazol-3-yl]pyrazolo[1,5-a]pyridin-2-yl]cyclopropanecarboxamide;
N-[5-[2-methyl-4-(4-piperidyloxy)pyrazol-3-yl]pyrazolo[1,5-a]pyridin-2-yl]cyclopropanecarboxamide;
N-[5-[4-[(3-ethyl-1-methyl-azetidin-3-yl)methoxy]-2-methyl-pyrazol-3-yl]pyrazolo[1,5-a]pyridin-2-yl]cyclopropanecarboxamide;
(rac-cis)-N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide;
N-[5-[2-methyl-4-[(1-methyl-3-phenyl-azetidin-3-yl)methoxy]pyrazol-3-yl]pyrazolo[1,5-a]pyridin-2-yl]cyclopropanecarboxamide;
(3*R,4*S)—N-[5-[4-(4-methoxy-1-methyl-pyrrolidin-3-yl)oxy-2-methyl-pyrazol-3-yl]pyrazolo[1,5-a]pyridin-2-yl]cyclopropanecarboxamide;
N-[5-[2-methyl-4-[[(2S)-pyrrolidin-2-yl]methoxy]pyrazol-3-yl]pyrazolo[1,5-a]pyridin-2-yl]cyclopropanecarboxamide;
(3*S,4*S)—N-[5-[2-methyl-4-(4-methylpyrrolidin-3-yl)oxy-pyrazol-3-yl]pyrazolo[1,5-a]pyridin-2-yl]cyclopropanecarboxamide;
(3*R,4*R)—N-[5-[4-(4-hydroxypyrrolidin-3-yl)oxy-2-methyl-pyrazol-3-yl]pyrazolo[1,5-a]pyridin-2-yl]cyclopropanecarboxamide;
N-[5-[4-[(3R,4S)-4-fluoro-1-methyl-pyrrolidin-3-yl]oxy-2-methyl-pyrazol-3-yl]pyrazolo[1,5-a]pyridin-2-yl]cyclopropanecarboxamide;
N-[5-[4-[[(3S,4S)-4-hydroxypyrrolidin-3-yl]methoxy]-2-methyl-pyrazol-3-yl]pyrazolo[1,5-a]pyridin-2-yl]cyclopropanecarboxamide;
N-[5-[4-[[(3S,4S)-4-hydroxy-1-methyl-pyrrolidin-3-yl]methoxy]-2-methyl-pyrazol-3-yl]pyrazolo[1,5-a]pyridin-2-yl]cyclopropanecarboxamide;
(3*R,4*R)—N-[5-[4-(1,4-dimethylpyrrolidin-3-yl)oxy-2-methyl-pyrazol-3-yl]pyrazolo[1,5-a]pyridin-2-yl]cyclopropanecarboxamide;
N-[5-[2-methyl-4-[[(2S)-1-methylpyrrolidin-2-yl]methoxy]pyrazol-3-yl]pyrazolo[1,5-a]pyridin-2-yl]cyclopropanecarboxamide;
N-[5-[4-[[(2S)-1-ethylpyrrolidin-2-yl]methoxy]-2-methyl-pyrazol-3-yl]pyrazolo[1,5-a]pyridin-2-yl]cyclopropanecarboxamide;
(rac-trans)-N-[5-[4-(4-methoxypyrrolidin-3-yl)oxy-2-methyl-pyrazol-3-yl]pyrazolo[1,5-a]pyridin-2-yl]cyclopropanecarboxamide;
N-[5-[4-[[(3S,4S)-4-methoxypyrrolidin-3-yl]methoxy]-2-methyl-pyrazol-3-yl]pyrazolo[1,5-a]pyridin-2-yl]cyclopropanecarboxamide;
N-[5-[4-[[(3S,4S)-4-methoxy-1-methyl-pyrrolidin-3-yl]methoxy]-2-methyl-pyrazol-3-yl]pyrazolo[1,5-a]pyridin-2-yl]cyclopropanecarboxamide;
N-[5-[4-[[(3R,4R)-4-hydroxy-1-methyl-pyrrolidin-3-yl]methoxy]-2-methyl-pyrazol-3-yl]pyrazolo[1,5-a]pyridin-2-yl]cyclopropanecarboxamide;
N-[5-[4-[[(2R)-1-(cyanomethyl)azetidin-2-yl]methoxy]-2-methyl-pyrazol-3-yl]pyrazolo[1,5-a]pyridin-2-yl]cyclopropanecarboxamide;
N-[5-[4-[(2R)-2-amino-3-hydroxy-3-methyl-butoxy]-2-methyl-pyrazol-3-yl]pyrazolo[1,5-a]pyridin-2-yl]cyclopropanecarboxamide;
N-[5-[4-[[(2R)-1-ethylpyrrolidin-2-yl]methoxy]-2-methyl-pyrazol-3-yl]pyrazolo[1,5-a]pyridin-2-yl]cyclopropanecarboxamide;
N-[5-[4-[[(2R)-1-(2-methoxyethyl)pyrrolidin-2-yl]methoxy]-2-methyl-pyrazol-3-yl]pyrazolo[1,5-a]pyridin-2-yl]cyclopropanecarboxamide;
N-[5-[4-[[(2R)-1-benzylazetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-[(1-methylpyrazol-3-yl)methyl]azetidin-2-yl]methoxy]pyrazol-3-yl]pyrazolo[1,5-a]pyridin-2-yl]cyclopropanecarboxamide;
N-[5-[2-methyl-4-[[(2R)-1-[2-(trifluoromethoxy)ethyl]azetidin-2-yl]methoxy]pyrazol-3-yl]pyrazolo[1,5-a]pyridin-2-yl]cyclopropanecarboxamide;
N-[5-[2-methyl-4-[[(1S,4R,5R)-3-methyl-3-azabicyclo[3.1.0]hexan-4-yl]methoxy]pyrazol-3-yl]pyrazolo[1,5-a]pyridin-2-yl]cyclopropanecarboxamide;
N-[5-[2-methyl-4-[[(2R)-1-(oxetan-3-ylmethyl)azetidin-2-yl]methoxy]pyrazol-3-yl]pyrazolo[1,5-a]pyridin-2-yl]cyclopropanecarboxamide;
N-[5-[2-methyl-4-[[1-(methylamino)cyclopropyl]methoxy]pyrazol-3-yl]pyrazolo[1,5-a]pyridin-2-yl]cyclopropanecarboxamide;
N-[5-[4-[[(2R)-1-(2-methoxy-2-methyl-propyl)azetidin-2-yl]methoxy]-2-methyl-pyrazol-3-yl]pyrazolo[1,5-a]pyridin-2-yl]cyclopropanecarboxamide;
N-[5-[2-methyl-4-[[(2R)-1-(2-pyridyl)azetidin-2-yl]methoxy]pyrazol-3-yl]pyrazolo[1,5-a]pyridin-2-yl]cyclopropanecarboxamide;
N-[5-[4-[[(2R,3S)-1,3-dimethylazetidin-2-yl]methoxy]-2-methyl-pyrazol-3-yl]pyrazolo[1,5-a]pyridin-2-yl]cyclopropanecarboxamide;
3-[[5-[2-[(2,6-dimethylpyrimidin-4-yl)amino]pyrazolo[1,5-a]pyridin-5-yl]-1-methyl-pyrazol-4-yl]oxymethyl]cyclobutanol;
(*S)-3-[[5-[2-[(2,6-dimethylpyrimidin-4-yl)amino]pyrazolo[1,5-a]pyridin-5-yl]-1-methyl-pyrazol-4-yl]oxymethyl]tetrahydrofuran-3-ol;
(*S)—N-(2,6-dimethylpyrimidin-4-yl)-5-[2-methyl-4-[(3-methyltetrahydrofuran-3-yl)methoxy]pyrazol-3-yl]pyrazolo[1,5-a]pyridin-2-amine;
(1*R, 2*S, 4*S)—N-(2,6-dimethylpyrimidin-4-yl)-5-[2-methyl-4-(7-oxabicyclo[2.2.1]heptan-3-yloxy)pyrazol-3-yl]pyrazolo[1,5-a]pyridin-2-amine;
N-(2,6-dimethylpyrimidin-4-yl)-5-[2-methyl-4-[(2R,3S)-2-methylpyrrolidin-3-yl]oxy-pyrazol-3-yl]pyrazolo[1,5-a]pyridin-2-amine;
(*S)—N-(2,6-dimethylpyrimidin-4-yl)-5-[2-methyl-4-(8-oxaspiro[4.4]nonan-7-ylmethoxy)pyrazol-3-yl]pyrazolo[1,5-a]pyridin-2-amine;
(*R)-1-cyclobutyl-2-[5-[2-[(2,6-dimethylpyrimidin-4-yl)amino]pyrazolo[1,5-a]pyridin-5-yl]-1-m ethyl-pyrazol-4-yl]oxy-ethanol;
(3*S,4*S)—N-(2,6-dimethylpyrimidin-4-yl)-5-[2-methyl-4-(4-methyltetrahydrofuran-3-yl)oxy-pyrazol-3-yl]pyrazolo[1,5-a]pyridin-2-amine;
N-(2,6-dimethylpyrimidin-4-yl)-5-[2-methyl-4-[[(2R,4S)-4-methylpyrrolidin-2-yl]methoxy]pyrazol-3-yl]pyrazolo[1,5-a]pyridin-2-amine;
(*S)—N-(2,6-dimethylpyrimidin-4-yl)-5-[2-methyl-4-[(2-methyltetrahydrofuran-2-yl)methoxy]pyrazol-3-yl]pyrazolo[1,5-a]pyridin-2-amine;
(1*S,2*S)-2-[[5-[2-[(2,6-dimethylpyrimidin-4-yl)amino]pyrazolo[1,5-a]pyridin-5-yl]-1-methyl-pyrazol-4-yl]oxymethyl]cyclobutanol;
5-[2-methyl-4-[(3S)-1-methylpyrrolidin-3-yl]oxy-pyrazol-3-yl]-N-pyridazin-3-yl-pyrazolo[1,5-a]pyridin-2-amine;
5-[2-methyl-4-[(3S)-1-methylpyrrolidin-3-yl]oxy-pyrazol-3-yl]-N-pyrazin-2-yl-pyrazolo[1,5-a]pyridin-2-amine;
5-[2-methyl-4-[(3S)-1-methylpyrrolidin-3-yl]oxy-pyrazol-3-yl]-N-(6-methyl-3-pyridyl)pyrazolo[1,5-a]pyridin-2-amine;
5-[2-methyl-4-[(3S)-1-methylpyrrolidin-3-yl]oxy-pyrazol-3-yl]-N-(2-pyridyl)pyrazolo[1,5-a]pyridin-2-amine;
5-[2-methyl-4-[(3S)-1-methylpyrrolidin-3-yl]oxy-pyrazol-3-yl]-N-[5-(trifluoromethyl)pyrazin-2-yl]pyrazolo[1,5-a]pyridin-2-amine;
5-[2-methyl-4-[(3S)-1-methylpyrrolidin-3-yl]oxy-pyrazol-3-yl]-N-(5-methylpyrazin-2-yl)pyrazolo[1,5-a]pyridin-2-amine;
5-[2-methyl-4-[(3S)-1-methylpyrrolidin-3-yl]oxy-pyrazol-3-yl]-N-pyrimidin-4-yl-pyrazolo[1,5-a]pyridin-2-amine;
5-[2-methyl-4-[(3S)-1-methylpyrrolidin-3-yl]oxy-pyrazol-3-yl]-N-(2-methylpyrimidin-4-yl)pyrazolo[1,5-a]pyridin-2-amine;
5-[2-methyl-4-[(3S)-1-methylpyrrolidin-3-yl]oxy-pyrazol-3-yl]-N-[5-(trifluoromethyl)pyridazin-3-yl]pyrazolo[1,5-a]pyridin-2-amine;
5-[2-methyl-4-[(3S)-1-methylpyrrolidin-3-yl]oxy-pyrazol-3-yl]-N-[2-(trifluoromethyl)pyrimidin-4-yl]pyrazolo[1,5-a]pyridin-2-amine;
5-[2-methyl-4-[(3S)-1-methylpyrrolidin-3-yl]oxy-pyrazol-3-yl]-N-[6-(trifluoromethyl)pyrimidin-4-yl]pyrazolo[1,5-a]pyridin-2-amine;
1-methyl-N-[5-[2-methyl-4-[(3S)-1-methylpyrrolidin-3-yl]oxy-pyrazol-3-yl]pyrazolo[1,5-a]pyridin-2-yl]pyrrolo[2,3-b]pyridin-5-amine;
N-[6-(methoxymethyl)pyrimidin-4-yl]-5-[2-methyl-4-[(3S)-1-methylpyrrolidin-3-yl]oxy-pyrazol-3-yl]pyrazolo[1,5-a]pyridin-2-amine;
N-(6-cyclopropylpyridazin-3-yl)-5-[2-methyl-4-[(3S)-1-methylpyrrolidin-3-yl]oxy-pyrazol-3-yl]pyrazolo[1,5-a]pyridin-2-amine;
N-[5-[2-methyl-4-(4-piperidyloxymethyl)pyrazol-3-yl]pyrazolo[1,5-a]pyridin-2-yl]cyclopropane carboxamide;
N-[5-[2-methyl-4-[[(3S)-1-methylpyrrolidin-3-yl]oxymethyl]pyrazol-3-yl]pyrazolo[1,5-a]pyridin-2-yl]cyclopropanecarboxamide;
(*S)—N-[5-[4-[(4,4-difluoropyrrolidin-3-yl)oxymethyl]-2-methyl-pyrazol-3-yl]pyrazolo[1,5-a]pyridin-2-yl]cyclopropanecarboxamide;
N-[5-[4-[[(3R,4R)-4-fluoropyrrolidin-3-yl]oxymethyl]-2-methyl-pyrazol-3-yl]pyrazolo[1,5-a]pyridin-2-yl]cyclopropanecarboxamide;
N-[5-[4-[[(3R,4R)-4-fluoro-1-methyl-pyrrolidin-3-yl]oxymethyl]-2-methyl-pyrazol-3-yl]pyrazolo[1,5-a]pyridin-2-yl]cyclopropanecarboxamide;
N-[5-[4-[(3-ethyl-1-methyl-azetidin-3-yl)oxymethyl]-2-methyl-pyrazol-3-yl]pyrazolo[1,5-a]pyridin-2-yl]cyclopropanecarboxamide;
(rac-trans)-5-[4-[(1,4-dimethylpyrrolidin-3-yl)oxymethyl]-2-methyl-pyrazol-3-yl]-N-(5-methylpyrazin-2-yl)pyrazolo[1,5-a]pyridin-2-amine;
N-[5-[2-(difluoromethyl)-4-[(3S)-1-methylpyrrolidin-3-yl]oxy-pyrazol-3-yl]pyrazolo[1,5-a]pyridin-2-yl]cyclopropanecarboxamide;
N-[6-[2-methyl-4-[(3S)-pyrrolidin-3-yl]oxy-pyrazol-3-yl]imidazo[1,2-a]pyridin-2-yl]cyclopropanecarboxamide;
N-[6-[2-methyl-4-[(3S)-1-methylpyrrolidin-3-yl]oxy-pyrazol-3-yl]imidazo[1,2-a]pyridin-2-yl]cyclopropanecarboxamide;
cis-N-[5-[2-methyl-4-[(1-methylazetidin-2-yl)methoxy]pyrazol-3-yl]pyrazolo[1,5-a]pyridin-2-yl]-2-phenyl-cyclopropanecarboxamide;
(1*R,2*S)—N-(5-(1-methyl-4-(((R)-1-methylazetidin-2-yl)methoxy)-1H-pyrazol-5-yl)pyrazolo[1,5-a]pyridin-2-yl)-2-phenylcyclopropane-1-carboxamide;
cis-2-cyano-N-[5-[2-methyl-4-[(1-methylazetidin-2-yl)methoxy]pyrazol-3-yl]pyrazolo[1,5-a]pyridin-2-yl]cyclopropanecarboxamide;
N-(2,6-dimethylpyrimidin-4-yl)-6-[4-[[(2R)-1-ethylazetidin-2-yl]methoxy]-2-methyl-pyrazol-3-yl]imidazo[1,2-a]pyridin-2-amine;
5-[2-(difluoromethyl)-4-[[(2R)-1-ethylazetidin-2-yl]methoxy]pyrazol-3-yl]-N-(2-methylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(2,6-dimethylpyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyridazin-3-yl)pyrazolo[1,5-a]pyridin-2-amine;
5-[2-(difluoromethyl)-4-[[(2R)-1-ethylazetidin-2-yl]methoxy]pyrazol-3-yl]-N-(2-methoxypyrimidin-4-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methylpyrazin-2-yl)pyrazolo[1,5-a]pyridin-2-amine;
5-[2-(difluoromethyl)-4-[[(2R)-1-ethylazetidin-2-yl]methoxy]pyrazol-3-yl]-N-(6-methoxy-5-methyl-pyridazin-3-yl)pyrazolo[1,5-a]pyridin-2-amine;
6-[[5-[2-(difluoromethyl)-4-[[(2R)-1-ethylazetidin-2-yl]methoxy]pyrazol-3-yl]pyrazolo[1,5-a]pyridin-2-yl]amino]-2,4-dimethyl-pyridazin-3-one;
N-(2,6-dimethylpyrimidin-4-yl)-6-[2-methyl-4-[[(2R)-1-methylazetidin-2-yl]methoxy]pyrazol-3-yl]imidazo[1,2-a]pyridin-2-amine;
N-[5-(5-methylisoxazol-4-yl)pyrazolo[1,5-a]pyridin-2-yl]cyclopropanecarboxamide;
N-[5-[2-methyl-4-[[(2S)-4-methylmorpholin-2-yl]methoxy]pyrazol-3-yl]pyrazolo[1,5-a]pyridin-2-yl]cyclopropanecarboxamide;
N-[5-[2-methyl-4-[(3R)-1-methylpyrrolidin-3-yl]oxy-pyrazol-3-yl]pyrazolo[1,5-a]pyridin-2-yl]cyclopropanecarboxamide;
N-[5-[2-(cyclopropylmethyl)-4-[[(3R)-1-methylpyrrolidin-3-yl]methoxy]pyrazol-3-yl]pyrazolo[1,5-a]pyridin-2-yl]cyclopropanecarboxamide;
N-[5-[4-[[(3R)-1-methylpyrrolidin-3-yl]methoxy]-2-propyl-pyrazol-3-yl]pyrazolo[1,5-a]pyridin-2-yl]cyclopropanecarboxamide;
N-[5-[2-benzyl-4-[[(3R)-1-methylpyrrolidin-3-yl]methoxy]pyrazol-3-yl]pyrazolo[1,5-a]pyridin-2-yl]cyclopropanecarboxamide;
N-[5-[2-methyl-4-[(1-methyl-4-piperidyl)oxy]pyrazol-3-yl]pyrazolo[1,5-a]pyridin-2-yl]cyclopropanecarboxamide;
N-[5-[5-methyl-3-[(1-methyl-4-piperidyl)oxy]isoxazol-4-yl]pyrazolo[1,5-a]pyridin-2-yl]cyclopropanecarboxamide;
N-[5-[5-methyl-3-(1-methylazetidin-3-yl)oxy-isoxazol-4-yl]pyrazolo[1,5-a]pyridin-2-yl]cyclopropanecarboxamide;
N-[5-[5-methyl-3-[(1-methylazetidin-3-yl)oxymethyl]isoxazol-4-yl]pyrazolo[1,5-a]pyridin-2-yl]cyclopropanecarboxamide;
N-[5-[5-methyl-3-[[(3R)-morpholin-3-yl]methoxy]isoxazol-4-yl]pyrazolo[1,5-a]pyridin-2-yl]cyclopropanecarboxamide;
N-[5-[5-methyl-3-[[(3S)-morpholin-3-yl]methoxy]isoxazol-4-yl]pyrazolo[1,5-a]pyridin-2-yl]cyclopropanecarboxamide;
N-[5-[5-methyl-3-[[(2S)-1-methylazetidin-2-yl]methoxy]isoxazol-4-yl]pyrazolo[1,5-a]pyridin-2-yl]cyclopropanecarboxamide;
N-[5-[4-[(3S,4R)-4-fluoro-1-methyl-pyrrolidin-3-yl]oxy-2-methyl-pyrazol-3-yl]pyrazolo[1,5-a]pyridin-2-yl]cyclopropanecarboxamide;
N-[5-[5-methyl-3-[[(2S)-1-methylpyrrolidin-2-yl]methoxy]isoxazol-4-yl]pyrazolo[1,5-a]pyridin-2-yl]cyclopropanecarboxamide;
N-[5-[5-methyl-3-[[(2S)-1-methyl-2-piperidyl]methoxy]isoxazol-4-yl]pyrazolo[1,5-a]pyridin-2-yl]cyclopropanecarboxamide;
N-[5-[5-methyl-3-[[(2R)-1-methyl-2-piperidyl]methoxy]isoxazol-4-yl]pyrazolo[1,5-a]pyridin-2-yl]cyclopropanecarboxamide;
5-[2-methyl-4-[[(2R)-1-methylazetidin-2-yl]methoxy]pyrazol-3-yl]pyrazolo[1,5-a]pyridin-2-amine;
N-[5-[4-[(3S,4R)-4-fluoropyrrolidin-3-yl]oxy-2-methyl-pyrazol-3-yl]pyrazolo[1,5-a]pyridin-2-yl]cyclopropanecarboxamide;
N-[5-[2-methyl-4-[[(2S)-1-methyl-2-piperidyl]methoxy]pyrazol-3-yl]pyrazolo[1,5-a]pyridin-2-yl]cyclopropanecarboxamide;
N-[5-[4-[(1-aminocyclopropyl)methoxy]-2-methyl-pyrazol-3-yl]pyrazolo[1,5-a]pyridin-2-yl]cyclopropanecarboxamide; and
N-[5-[2-methyl-4-[[(2R)-1-methyl-2-piperidyl]methoxy]pyrazol-3-yl]pyrazolo[1,5-a]pyridin-2-yl]cyclopropanecarboxamide;
or a tautomer, stereoisomer pharmaceutically acceptable salt, or solvate thereof.

16. A compound selected from the group consisting of:
N-[5-[2-(cyclopropylmethyl)-4-[[(3R)-1-methylpyrrolidin-3-yl]methoxy]pyrazol-3-yl]pyrazolo[1,5-a]pyridin-2-yl]cyclopropanecarboxamide;
N-[5-[2-benzyl-4-[[(3R)-1-methylpyrrolidin-3-yl]methoxy]pyrazol-3-yl]pyrazolo[1,5-a]pyridin-2-yl]cyclopropanecarboxamide; and
N-[5-[5-methyl-3-[(1-methylazetidin-3-yl)oxymethyl]isoxazol-4-yl]pyrazolo[1,5-a]pyridin-2-yl]cyclopropanecarboxamide;
or a tautomer, stereoisomer pharmaceutically acceptable salt, or solvate thereof.

17. A pharmaceutical composition comprising the compound of claim 1, and a pharmaceutically acceptable carrier.

18. A method of inhibiting a salt inducible kinase (SIK) in a subject in need thereof, the method comprising administering to the subject the pharmaceutical composition of claim 17.

19. The method of claim 18, wherein the subject is in need of a treatment of a disease, disorder, or condition mediated by a SIK, such as an autoimmune disorder or a proliferative disorder.
